(19) United States
(12) Patent Application Publication
(10) Pub. No.: US 2026/0027097 A1
(43) Pub. Date: Jan. 29, 2026

(54) METHODS AND COMPOUNDS FOR RESTORING MUTANT p53 FUNCTION

(71) Applicant: PMV Pharmaceuticals, Inc., Princeton, NJ (US)

(72) Inventors: Melissa Dumble, Watchung, NJ (US); Anna Puzio-Kuter, Hillsborough, NJ (US)

(21) Appl. No.: 18/854,275

(22) PCT Filed: Apr. 7, 2023
(86) PCT No.: PCT/US2023/065549

§ 371 Date: Oct. 4, 2024

Related U.S. Application Data
(60) Provisional application No. 63/329,080, filed on Apr. 8, 2022.

(51) Int. Cl. A61K 31/445; A61K 45/06; A61P 35/00; C12Q 1/6881

(52) U.S. Cl. CPC A61K 31/445; A61K 45/06; A61P 35/00; C12Q 1/6881; C12Q 2600/112

(57) ABSTRACT

Mutations in oncogenes and tumor suppressors contribute to the development and progression of cancer. The present disclosure describes compounds and methods to recover wild-type function to p53 mutants. The compounds of the present invention can bind to mutant p53 and restore the ability of the p53 mutant to bind DNA and activate downstream effectors involved in tumor suppression. The disclosed compounds can be used to reduce the progression of cancers that contain a p53 mutation.

### CROSS REFERENCE

This application claims the benefit of U.S. Provisional Application No. 63/329,080, filed Apr. 8, 2022, which is incorporated by reference herein in its entirety.

### BACKGROUND

Cancer, an uncontrolled proliferation of cells, is a multifactorial disease characterized by tumor formation, growth, and in some instances, metastasis. Cells carrying an activated oncogene, damaged genome, or other cancer-promoting alterations can be prevented from replicating through an elaborate tumor suppression network. A central component of this tumor suppression network is p53, one of the most potent tumor suppressors in the cell. Both the wild type and mutant conformations of p53 are implicated in the progression of cancer.

### INCORPORATION BY REFERENCE

Each patent, publication, and non-patent literature cited in the application is hereby incorporated by reference in its entirety as if each was incorporated by reference individually.

### SUMMARY OF THE INVENTION

In some embodiments, disclosed herein is a method comprising: a) administering a therapeutically-effective amount of a compound to a subject in need thereof, wherein the compound binds a mutant p53 protein and increases wild-type p53 activity of the mutant p53 protein; and b) after the administering, observing that the administering increases an immune response in the subject against a cancer.

In some embodiments, disclosed herein is a method of treating a cancer comprising: a) administering a therapeutically-effective amount of a compound to a subject in need thereof, wherein the compound binds a mutant p53 protein and increases wild-type p53 activity of the mutant p53 protein; and b) after the administering, performing a gene expression assay on the subject, an increase is observed in an immune response gene signature or a check-point biomarker signature.

In some embodiments, disclosed herein is a method comprising: a) determining that a subject is in need of an increase in a gene signature; and b) based on the determining, administering a therapeutically-effective amount of a compound to the subject in need thereof, wherein the compound binds a mutant p53 protein and increases wild-type p53 activity of the mutant p53 protein.

### BRIEF DESCRIPTION OF THE FIGURES

FIG. 1 PANEL A shows the dosing and tumor harvesting timeline of EXAMPLE 2. FIG. 1 PANEL B shows the dosing and tumor harvesting timeline of EXAMPLE 3.

FIG. 2 PANEL A-PANEL F show changes in percentage of population of CD4+ T-cells (PANEL A), CD8+ cells (PANEL B), T-reg cells (PANEL C), NKT cells (PANEL D), g-MDSC cells (PANEL E), and M2 macrophages (PANEL F) compared to CD45+ in mice treated with vehicle, 75 mg/kg, 150 mg/kg, and 300 mg/kg of Compound 1 for 2Q7Dx1, 2Q7Dx2, and 2Q7Dx3.

FIG. 3 PANEL A-PANEL F show changes in percentage of population of CD4+ T-cells (PANEL A), CD8+ cells (PANEL B), T-reg cells (PANEL C), NKT cells (PANEL D), g-MDSC cells (PANEL E), and M2 macrophages (PANEL F) compared to CD45+ in mice treated with vehicle, 25 mg/kg, 50 mg/kg, and 100 mg/kg of Compound 2 for QDx4, QDx10, and QDx17.

FIG. 4 PANEL A-PANEL F show changes in NanoString signature scores for CD45 cell signature analysis (PANEL A), T-cell signature analysis (PANEL B), exhausted CD8 signature analysis (PANEL C), tumor inflammation signature analysis (PANEL D), PD1 signature analysis (PANEL E), and PDL1 signature analysis (PANEL F) in mice treated with vehicle, 150 mg/kg and 300 mg/kg of Compound 1 at 2Q7Dx2 and 2Q7Dx3; or 100 mg/kg of Compound 1 at QDx10 and QDx17.

### DETAILED DESCRIPTION

The present invention provides compounds and methods for restoring wild-type function to mutant p53. The compounds of the present invention can bind to mutant p53 and restore the ability of the p53 mutant to bind DNA. The restoration of activity of the p53 mutant can allow for the activation of downstream effectors of p53 leading to inhibition of cancer progression. The invention further provides methods of treatment of a cancerous lesion or a tumor harboring a p53 mutation.

Cancer is a collection of related diseases characterized by uncontrolled proliferation of cells with the potential to metastasize throughout the body. Cancer can be classified into five broad categories including, for example: carcinomas, which can arise from cells that cover internal and external parts of the body such as the lung, breast, and colon; sarcomas, which can arise from cells that are located in bone, cartilage, fat, connective tissue, muscle, and other supportive tissues; lymphomas, which can arise in the lymph nodes and immune system tissues; leukemia, which can arise in the bone marrow and accumulate in the bloodstream; and adenomas, which can arise in the thyroid, the pituitary gland, the adrenal gland, and other glandular tissues.

Although different cancers can develop in virtually any of the body's tissues, and contain unique features, the basic processes that cause cancer can be similar in all forms of the disease. Cancer begins when a cell breaks free from the normal restraints on cell division and begins to grow and divide out of control. Genetic mutations in the cell can preclude the ability of the cell to repair damaged DNA or initiate apoptosis, and can result in uncontrolled growth and division of cells.

The ability of tumor cell populations to multiply is determined not only by the rate of cell proliferation but also by the rate of cell attrition. Programmed cell death, or apoptosis, represents a major mechanism of cellular attrition. Cancer cells can evade apoptosis through a variety of strategies, for example, through the suppression of p53 function, thereby suppressing expression of pro-apoptotic proteins.

Oncogenes and tumor suppressor genes can regulate the proliferation of cells. Genetic mutations can affect oncogenes and tumor suppressors, potentially activating or suppressing activity abnormally, further facilitating uncontrolled cell division. Whereas oncogenes assist in cellular growth, tumor suppressor genes slow cell division by repairing damaged DNA and activating apoptosis. Cellular oncogenes that can be mutated in cancer include, for example, Cdk1, Cdk2, Cdk3, Cdk4, Cdk6, EGFR, PDGFR, VEGF, HER2, Raf kinase, K-Ras, and myc. Tumor suppressor genes that can be mutated in cancer include, for example, BRCA1, BRCA2, cyclin-dependent kinase inhibitor 1C, Retinoblastoma protein (pRb), PTEN, p16, p27, p53, and p73.

### Tumor Suppressor p53.

The tumor suppressor protein p53 is a 393 amino acid transcription factor that can regulate cell growth in response to cellular stresses including, for example, UV radiation, hypoxia, oncogene activation, and DNA damage. p53 has various mechanisms for inhibiting the progression of cancer including, for example, initiation of apoptosis, maintenance of genomic stability, cell cycle arrest, induction of senescence, and inhibition of angiogenesis. Due to the critical role of p53 in tumor suppression, p53 is inactivated in almost all cancers either by direct mutation or through perturbation of associated signaling pathways involved in tumor suppression. Homozygous loss of the p53 gene occurs in almost all types of cancer, including carcinomas of the breast, colon, and lung. The presence of certain p53 mutations in several types of human cancer can correlate with less favorable patient prognosis.

In the absence of stress signals, p53 levels are maintained at low levels via the interaction of p53 with Mdm2, an E3 ubiquitin ligase. In an unstressed cell, Mdm2 can target p53 for degradation by the proteasome. Under stress conditions, the interaction between Mdm2 and p53 is disrupted, and p53 accumulates. The critical event leading to the activation of p53 is phosphorylation of the N-terminal domain of p53 by protein kinases, thereby transducing upstream stress signals. The phosphorylation of p53 leads to a conformational change, which can promote DNA binding by p53 and allow transcription of downstream effectors. The activation of p53 can induce, for example, the intrinsic apoptotic pathway, the extrinsic apoptotic pathway, cell cycle arrest, senescence, and DNA repair. p53 can activate proteins involved in the above pathways including, for example, Fas/Apo1, KILLER/DR5, Bax, Puma, Noxa, Bid, caspase-3, caspase-6, caspase-7, caspase-8, caspase-9, and p21 (WAF1). Additionally, p53 can repress the transcription of a variety of genes including, for example, c-MYC, Cyclin B, VEGF, RAD51, and hTERT.

Each chain of the p53 tetramer is composed of several functional domains including the transactivation domain (amino acids 1-100), the DNA-binding domain (amino acids 101-306), and the tetramerization domain (amino acids 307-355), which are highly mobile and largely unstructured. Most p53 cancer mutations are located in the DNA-binding core domain of the protein, which contains a central β-sandwich of anti-parallel β-sheets that serves as a basic scaffold for the DNA-binding surface. The DNA-binding surface is composed of two β-turn loops, L2 and L3, which are stabilized by a zinc ion, for example, at Arg175 and Arg248, and a loop-sheet-helix motif. Altogether, these structural elements form an extended DNA-binding surface that is rich in positively-charged amino acids, and makes specific contact with various p53 response elements.

Due to the prevalence of p53 mutations in virtually every type of cancer, the reactivation of wild type p53 function in a cancerous cell can be an effective therapy. Mutations in p53 located in the DNA-binding domain of the protein or periphery of the DNA-binding surface result in aberrant protein folding required for DNA recognition and binding. Mutations in p53 can occur, for example, at amino acids Val143, His168, Arg175, Tyr220, Gly245, Arg248, Arg249, Phe270, Arg273, and Arg282. p53 mutations that can abrogate the activity of p53 include, for example, R175H, Y220C, G245S, R248Q, R248W, R273H, and R282H. These p53 mutations can either distort the structure of the DNA-binding site or thermodynamically destabilize the folded protein at body temperature. Wild-type function of p53 mutants can be recovered by binding of the p53 mutant to a compound that can shift the folding-unfolding equilibrium towards the folded state, thereby reducing the rate of unfolding and destabilization.

Non-limiting examples of amino acids include: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); and valine (V, Val).

### Mechanism of Compounds of the Invention.

The compounds of the present invention can selectively bind to a p53 mutant and can recover wild-type activity of the p53 mutant including, for example, DNA binding function and activation of downstream targets involved in tumor suppression. In some embodiments, a compound of the invention selectively binds to the p53 Y220C mutant. The Y220C mutant is a temperature sensitive mutant, which binds to DNA at lower temperature and is denatured at body temperature. A compound of the invention can stabilize the Y220C mutant to reduce the likelihood of denaturation of the protein at body temperature.

In some embodiments, the compounds of the disclosure stabilize a mutant p53 and allows the mutant p53 to bind to DNA, thereby shifting the equilibrium of wild type and mutant p53 proteins to wild type p53. In some embodiments, the compounds of the disclosure reactivate the mutant p53 protein to provide wild type p53 activity. In some embodiments, the compounds of the disclosure reactivate the mutant p53 protein to provide pro-apoptotic p53 activity. In some embodiments, the compounds of the disclosure reactivate the mutant p53 protein to block angiogenesis. In some embodiments, the compounds of the disclosure reactivate the mutant p53 protein to induce cellular senescence. In some embodiments, the compounds of the disclosure reactivate the mutant p53 protein to induce cell cycle arrest.

The compounds of the disclosure can reconform mutant p53 to a conformation of p53 that exhibits anti-cancer activity. In some embodiments, the mutant p53 is reconformed to a wild type conformation p53. In some embodiments, the mutant p53 is reconformed to a pro-apoptotic conformation of p53. In some embodiments, the mutant p53 is reconformed to a conformation of p53 that blocks angiogenesis. In some embodiments, the mutant p53 is reconformed to a conformation of p53 that induces cellular senescence. In some embodiments, the mutant p53 is reconformed to a conformation of p53 that induces cell-cycle arrest.

Located in the periphery of the p53 β-sandwich connecting β-strands S7 and S8, the aromatic ring of Y220 is an integral part of the hydrophobic core of the β-sandwich. The Y220C mutation can be highly destabilizing, due to the formation of an internal surface cavity. A compound of the invention can bind to and occupy this surface crevice to stabilize the β-sandwich, thereby restoring wild-type p53 DNA-binding activity.

To determine the ability of a compound of the invention to bind and stabilize mutant p53, assays can be employed to detect, for example, a conformational change in the p53 mutant or activation of wild-type p53 targets. Conformational changes in p53 can be measured by, for example, differential scanning fluorimetry (DSF), isothermal titration calorimetry (ITC), nuclear magnetic resonance spectrometry (NMR), or X-ray crystallography. Additionally, antibodies specific for the wild type of mutant conformation of p53 can be used to detect a conformational change via, for example, immunoprecipitation (IP), immunofluorescence (IF), or immunoblotting.

Methods used to detect the ability of the p53 mutant to bind DNA can include, for example, DNA affinity immunoblotting, modified enzyme-linked immunosorbent assay (ELISA), electrophoretic mobility shift assay (EMSA), fluorescence resonance energy transfer (FRET), homogeneous time-resolved fluorescence (HTRF), and a chromatin immunoprecipitation (ChIP) assay.

To determine whether a compound described herein is able to reactivate the transcriptional activity of p53, the activation of downstream targets in the p53 signaling cascade can be measured. Activation of p53 effector proteins can be detected by, for example, immunohistochemistry (IHC-P), reverse transcription polymerase chain reaction (RT-PCR), and western blotting. The activation of p53 can also be measured by the induction of apoptosis via the caspase cascade and using methods including, for example, Annexin V staining, TUNEL assays, pro-caspase and caspase levels, and cytochrome c levels. Another consequence of p53 activation is senescence, which can be measured using methods such as β-galactosidase staining.

A p53 mutant that can be used to determine the effectiveness of a compound of the invention to increase the DNA binding ability of a p53 mutant is a p53 truncation mutant, which contains only amino acids 94-312, encompassing the DNA-binding domain of p53. For example, the sequence of the p53 Y220C mutant used for testing compound efficacy can be:

(SEQ ID NO. 1)
SSSVPSQ KTYQGSYGFR LGFLHSGTAK SVTCTYSPAL
NKMFCQLAKT CPVQLWVDST PPPGTRVRAM AIYKQSQHMT
EVVRRCPHHE RCSDSDGLAP POHLIRVEGN LRVEYLDDRN
TFRHSVVVPC EPPEVGSDCT TIHYNYMCNS SCMGGMNRRP
ILTIITLEDS SGNLLGRNSF EVHVCACPGR DRRTEEENLR
KKGEPHHELP PGSTKRALSN NT

A compound of the invention can increase the ability of a p53 mutant to bind DNA by at least or up to about 0.1%, at least or up to about 0.2%, at least or up to about 0.3%, at least or up to about 0.4%, at least or up to about 0.5%, at least or up to about 0.6%, at least or up to about 0.7%, at least or up to about 0.8%, at least or up to about 0.9%, at least or up to about 1%, at least or up to about 2%, at least or up to about 3%, at least or up to about 4%, at least or up to about 5%, at least or up to about 6%, at least or up to about 7%, at least or up to about 8%, at least or up to about 9%, at least or up to about 10%, at least or up to about 11%, at least or up to about 12%, at least or up to about 13%, at least or up to about 14%, at least or up to about 15%, at least or up to about 16%, at least or up to about 17%, at least or up to about 18%, at least or up to about 19%, at least or up to about 20%, at least or up to about 21%, at least or up to about 22%, at least or up to about 23%, at least or up to about 24%, at least or up to about 25%, at least or up to about 26%, at least or up to about 27%, at least or up to about 28%, at least or up to about 29%, at least or up to about 30%, at least or up to about 31%, at least or up to about 32%, at least or up to about 33%, at least or up to about 34%, at least or up to about 35%, at least or up to about 36%, at least or up to about 37%, at least or up to about 38%, at least or up to about 39%, at least or up to about 40%, at least or up to about 41%, at least or up to about 42%, at least or up to about 43%, at least or up to about 44%, at least or up to about 45%, at least or up to about 46%, at least or up to about 47%, at least or up to about 48%, at least or up to about 49%, at least or up to about 50%, at least or up to about 51%, at least or up to about 52%, at least or up to about 53%, at least or up to about 54%, at least or up to about 55%, at least or up to about 56%, at least or up to about 57%, at least or up to about 58%, at least or up to about 59%, at least or up to about 60%, at least or up to about 61%, at least or up to about 62%, at least or up to about 63%, at least or up to about 64%, at least or up to about 65%, at least or up to about 66%, at least or up to about 67%, at least or up to about 68%, at least or up to about 69%, at least or up to about 70%, at least or up to about 71%, at least or up to about 72%, at least or up to about 73%, at least or up to about 74%, at least or up to about 75%, at least or up to about 76%, at least or up to about 77%, at least or up to about 78%, at least or up to about 79%, at least or up to about 80%, at least or up to about 81%, at least or up to about 82%, at least or up to about 83%, at least or up to about 84%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, at least or up to about 100%, at least or up to about 125%, at least or up to about 150%, at least or up to about 175%, at least or up to about 200%, at least or up to about 225%, or at least or up to about 250% as compared to the ability of the p53 mutant to bind DNA in the absence of a compound of the invention.

A compound described herein can increase the activity of the p53 mutant that is, for example, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 11-fold, at least or up to about 12-fold, at least or up to about 13-fold, at least or up to about 14-fold, at least or up to about 15-fold, at least or up to about 16-fold, at least or up to about 17-fold, at least or up to about 18-fold, at least or up to about 19-fold, at least or up to about 20-fold, at least or up to about 25-fold, at least or up to about 30-fold, at least or up to about 35-fold, at least or up to about 40-fold, at least or up to about 45-fold, at least or up to about 50-fold, at least or up to about 55-fold, at least or up to about 60-fold, at least or up to about 65-fold, at least or up to about 70-fold, at least or up to about 75-fold, at least or up to about 80-fold, at least or up to about 85-fold, at least or up to about 90-fold, at least or up to about 95-fold, at least or up to about 100-fold, at least or up to about 110-fold, at least or up to about 120-fold, at least or up to about 130-fold, at least or up to about 140-fold, at least or up to about 150-fold, at least or up to about 160-fold, at least or up to about 170-fold, at least or up to about 180-fold, at least or up to about 190-fold, at least or up to about 200-fold, at least or up to about 250-fold, at least or up to about 300-fold, at least or up to about 350-fold, at least or up to about 400-fold, at least or up to about 450-fold, at least or up to about 500-fold, at least or up to about 550-fold, at least or up to about 600-fold, at least or up to about 650-fold, at least or up to about 700-fold, at least or up to about 750-fold, at least or up to about 800-fold, at least or up to about 850-fold, at least or up to about 900-fold, at least or up to about 950-fold, at least or up to about 1,000-fold, at least or up to about 1,500-fold, at least or up to about 2,000-fold, at least or up to about 3,000-fold, at least or up to about 4,000-fold, at least or up to about 5,000-fold, at least or up to about 6,000-fold, at least or up to about 7,000-fold, at least or up to about 8,000-fold, at least or up to about 9,000-fold, or at least or up to about 10,000-fold greater than the activity of the p53 mutant in the absence of the compound.

A compound of the invention can be used, for example, to induce apoptosis, cell cycle arrest, or senescence in a cell. In some embodiments, the cell is a cancer cell. In some embodiments, the cell carries a mutation in p53.

### Compounds of the Invention.

In some embodiments, a compound of the disclosure comprises a substituted heterocyclyl group, wherein the compound binds a mutant p53 protein and increases wild-type p53 activity of the mutant protein. In some embodiments, a compound of the disclosure comprises a heterocyclyl group comprising a halo substituent, wherein the compound binds a mutant p53 protein and increases wild-type p53 activity of the mutant protein. In some embodiments, the compound further comprises an indole group. In some embodiments, the indole group has a 1,1,1-trifluoroethyl substituent at a 1-position of the indole group.

In some embodiments, the indole group has a propargyl substituent at a 2-position of the indole group. In some embodiments, the propargyl substituent is attached to the indole group via an sp carbon atom of the propargyl substituent. In some embodiments, the propargyl substituent is attached to a nitrogen atom of an aniline group via a methylene group of the propargyl substituent. In some embodiments, the indole group comprises an amino substituent at a 4-position of the indole group. In some embodiments, the amino substituent is attached to the heterocyclyl group. In some embodiments, the heterocyclyl group is a piperidine group. In some embodiments, the halo substituent is a fluoro group. In some embodiments, the halo substituent is a chloro group. In some embodiments, the compound has oral bioavailability that is at least about 50% greater than that of an analogous compound that lacks the halo substituent on the heterocyclyl group.

In some embodiments, the compound is optionally substituted with at least one deuterium atom or one deuterio [D] group.

Non-limiting examples of compounds of the invention include compounds of any of the following formulae:

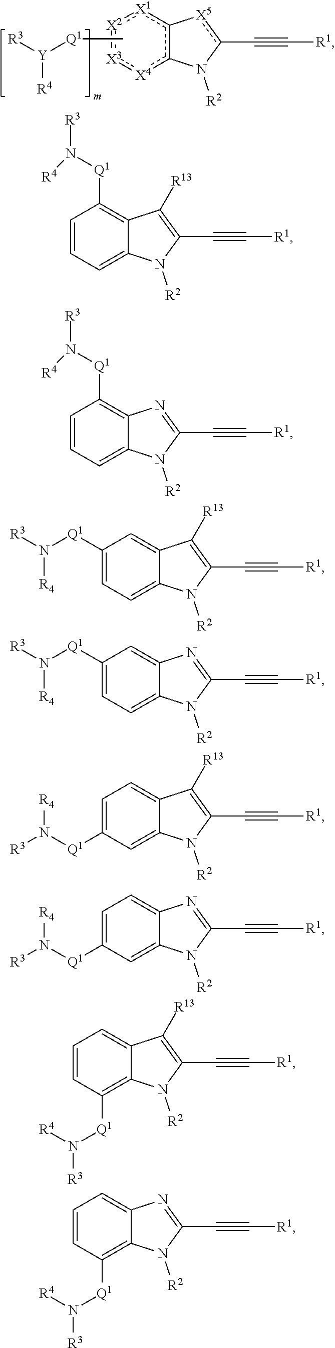

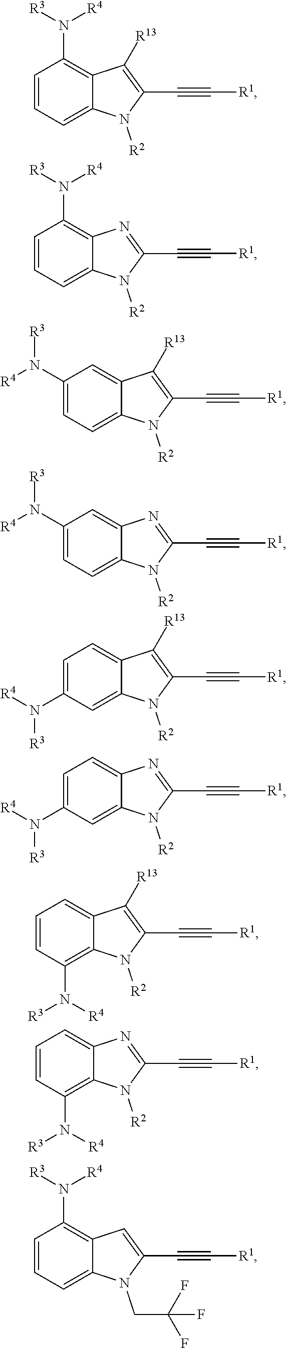

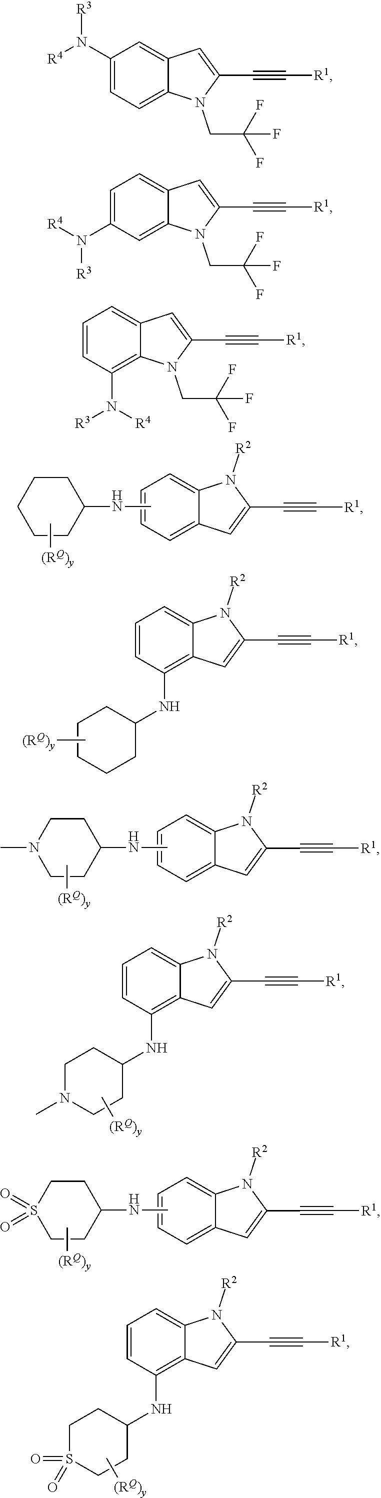

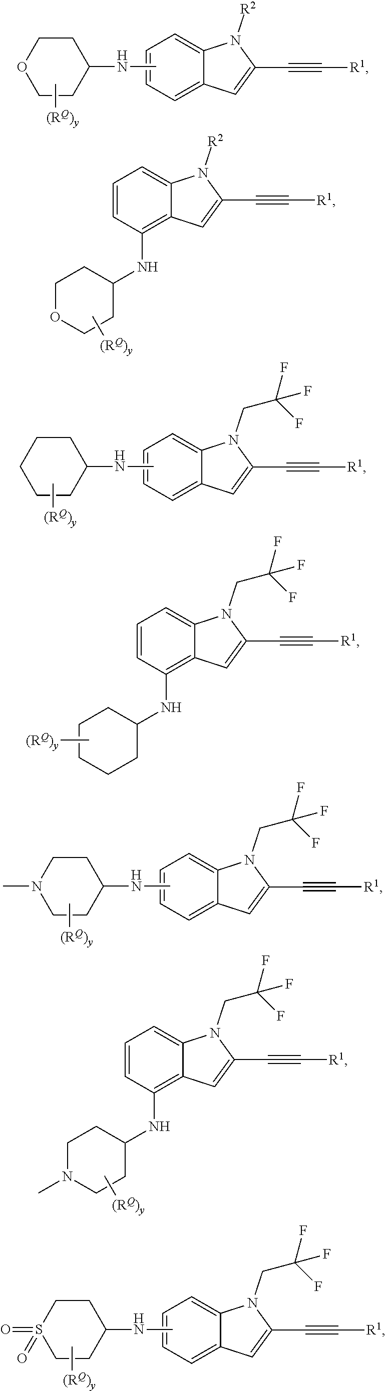

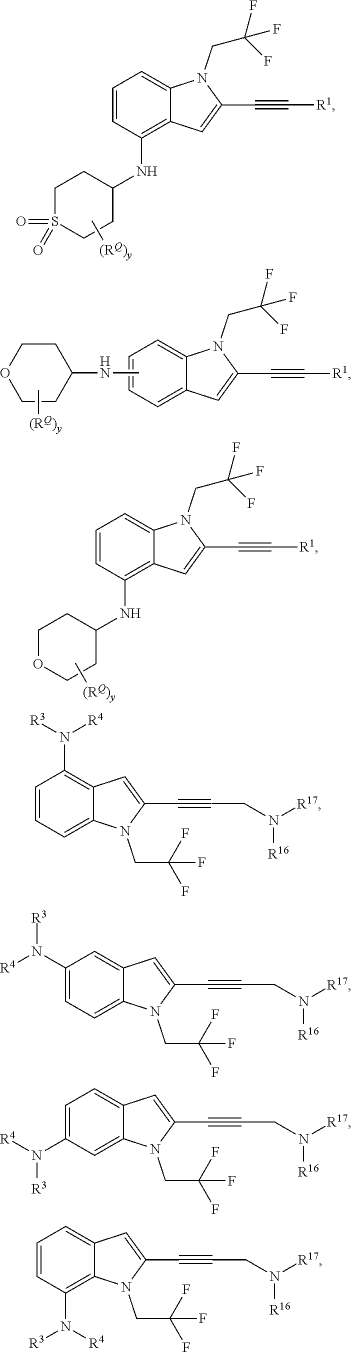

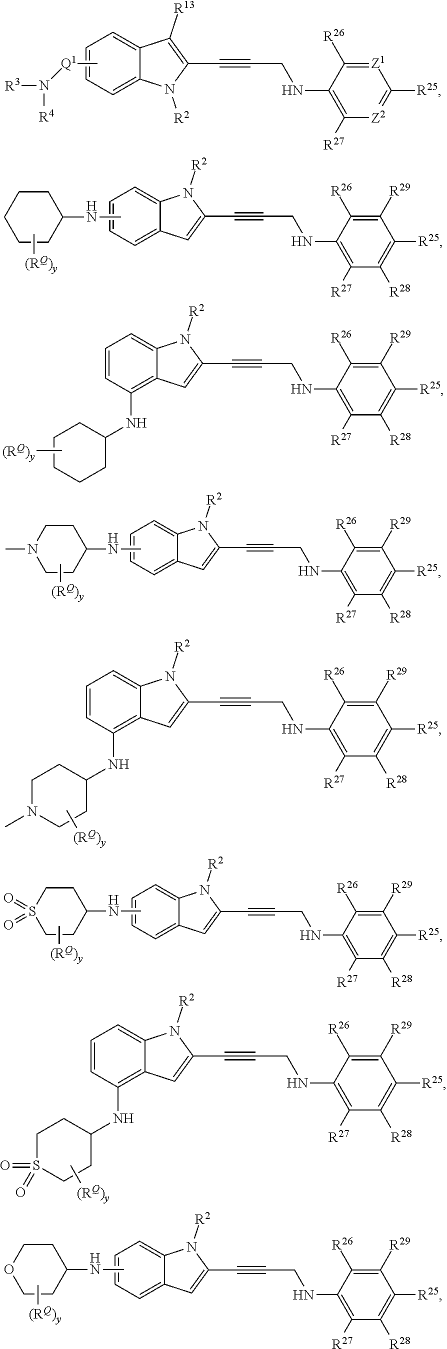

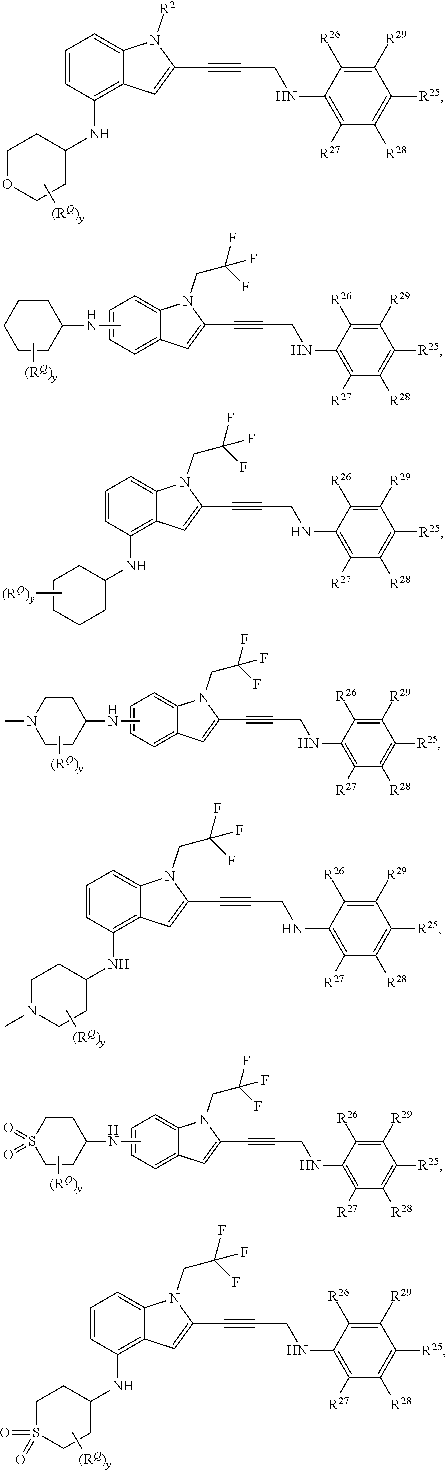

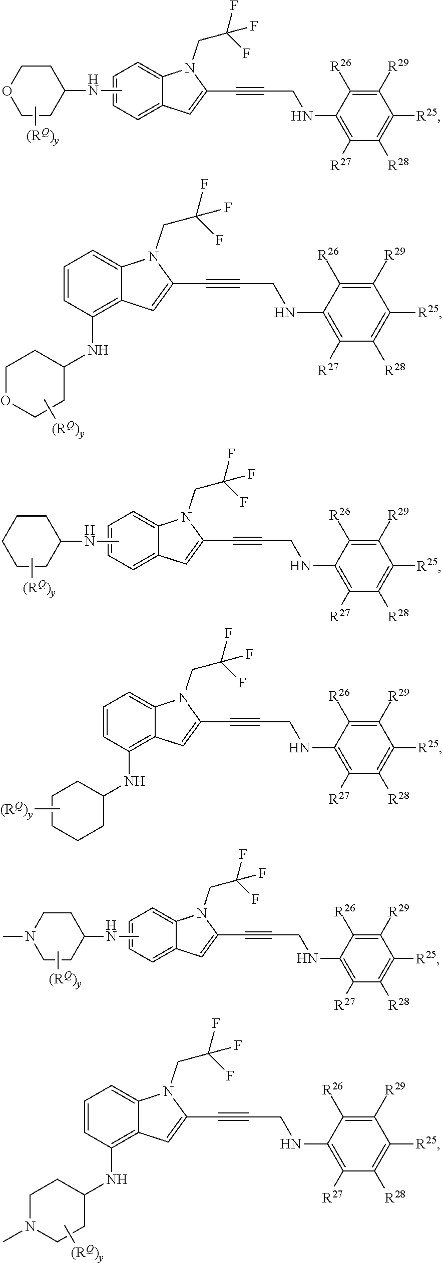

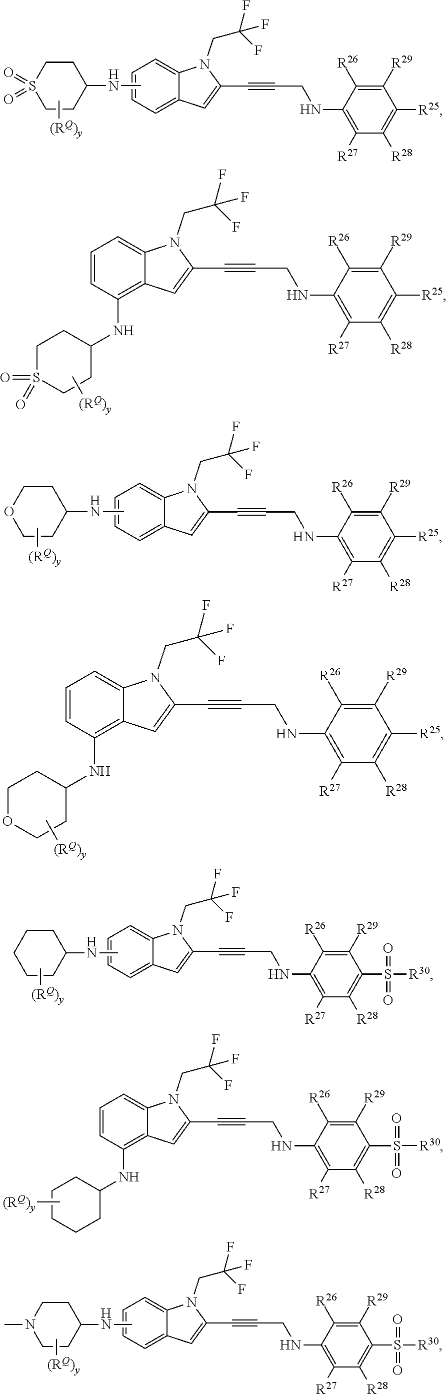

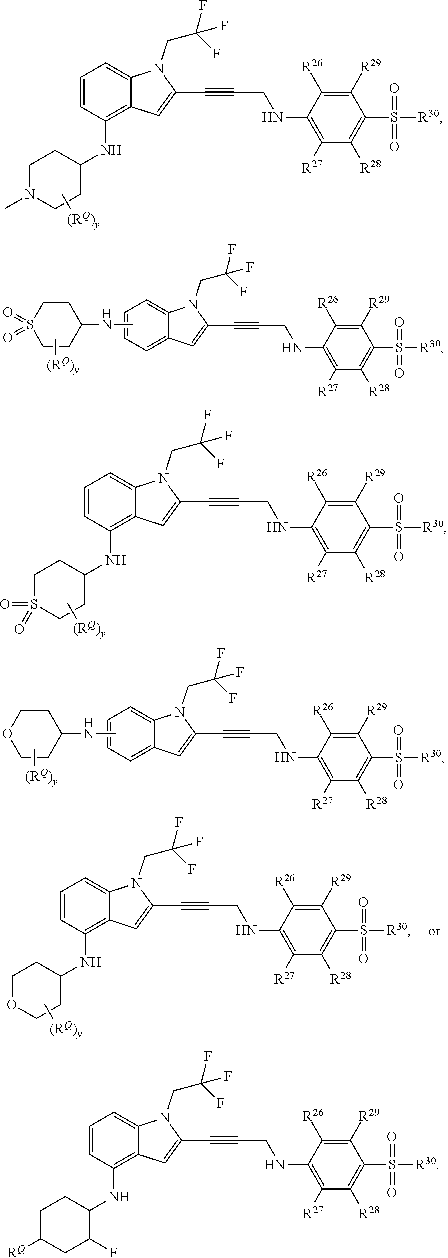

In some embodiments, the compound is of the formula:

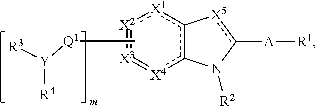

wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
   wherein at least one of X¹, X², X³, and X⁴ is a carbon atom connected to Q¹;
A is a linking group;
Q¹ is alkylene, alkenylene, or alkynylene, each of which is independently substituted or unsubstituted, or C═O, C═S, C═CR¹⁴R¹⁵, C═NR¹⁴, or a bond;
m is 1, 2, 3, or 4;
Y is N, O, or absent;
R¹ is alkyl, alkenyl, alkynyl, alkoxy, aryl, heteroaryl, or heterocyclyl, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, or hydrogen or halogen;
each R³ and R⁴ is independently alkyl, alkenyl, alkynyl, aryl, heteroaryl, or heterocyclyl, each of which is independently substituted or unsubstituted, or —C(O)R¹⁹, —C(O)OR¹⁹, —C(O)NR¹⁹R²⁰, —SOR¹⁹, —SO₂R¹⁹, or hydrogen, or R³ and R⁴ together with the atom to which R³ and R⁴ are bound form a ring, wherein the ring is substituted or unsubstituted, or R³ is absent;
each R², R⁵, R⁶, R⁷, R⁸, R⁹, R¹⁰, R¹¹, R, R¹³, R¹⁴, R¹⁵, R¹⁶, R¹, and R¹⁸ is independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹, or hydrogen or halogen;
each R¹⁹ and R²⁰ is independently alkyl, alkenyl, alkynyl, aryl, heteroaryl, or heterocyclyl, each of which is independently substituted or unsubstituted, or —C(O)R²³, —C(O)OR²³, —C(O)NR²³R²⁴, —OR²³, —SR²³, —NR²³R²⁴, —NR²³C(O)R²⁴, —OC(O)R²³, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, A is alkylene, alkenylene, or alkynylene, each of which is substituted or unsubstituted. In some embodiments, A is alkylene. In some embodiments, A is alkenylene. In some embodiments, A is alkynylene.

In some embodiments, A is aryl, heteroaryl, or heterocyclyl, each of which is substituted or unsubstituted. In some embodiments, A is substituted aryl. In some embodiments, A is substituted heteroaryl. In some embodiments, A is substituted heterocyclyl.

In some embodiments, R¹ is alkyl or alkenyl, each of which is unsubstituted or substituted, or —C(O)R¹⁶, —C(O)OR¹⁶, or —C(O)NR¹⁶R¹⁷. In some embodiments, R¹ is substituted alkyl. In some embodiments, R¹ is alkyl substituted with NR¹⁶R¹⁷.

In some embodiments, the compound is optionally substituted with at least one deuterium atom or deuterio [D] group.

In some embodiments, the compound of the formula is:

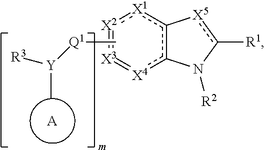

wherein
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
   wherein at least one of X¹, X², X³, and X⁴ is a carbon atom connected to Q¹;
Q¹ is alkylene, alkenylene, or alkynylene, each of which is independently substituted or unsubstituted, or C═O, C═S, C═CR¹⁴R¹⁵, C═NR¹⁴, or a bond;
m is 1, 2, 3, or 4;
Y is N, O, or absent;
ring A is a cyclic group;
R¹ is alkyl, alkenyl, alkynyl, aryl, heteroaryl, or heterocyclyl, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, C═O, C═S, —CN, —SiR¹⁶R¹⁷R¹⁸, or hydrogen;
R³ is alkyl, alkylene, alkenyl, alkenylene, alkynyl, aryl, heteroaryl, or heterocyclyl, each of which is independently substituted or unsubstituted, or —C(O)R¹⁹, —C(O)OR¹⁹, —C(O)NR¹⁹R²⁰, —SOR¹⁹, —SO₂R¹⁹, or hydrogen, or R³ and A together with the atom to which R³ and A are bound form a ring, wherein the ring is substituted or unsubstituted, or R³ is absent,
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR¹, —NR²¹R²², —NR²¹C(O)R²², —OC(O)R²¹, or hydrogen or halogen;
each R¹⁹ and R²⁰ is independently alkyl, alkenyl, alkynyl, aryl, heteroaryl, or heterocyclyl, each of which is independently substituted or unsubstituted, or —C(O)R²³, —C(O)OR²³, —C(O)NR²³R²⁴, —OR²³, —SR²³, —NR²³R²⁴, —NR²³C(O)R²⁴, —OC(O)R²³, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen, or a pharmaceutically-acceptable salt thereof.

In some embodiments, a compound of the invention is a compound of the formula

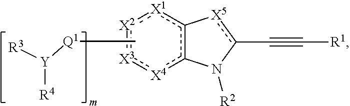

wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;

wherein at least one of X¹, X², X³, and X⁴ is a carbon atom connected to Q¹;
Q¹ is alkylene, alkenylene, or alkynylene, each of which is independently substituted or unsubstituted, or C═O, C═S, C═CR¹⁴R¹⁵, C═NR¹⁴, or a bond;
m is 1, 2, 3, or 4;
Y is N, O, or absent;
R¹ is alkyl, alkenyl, alkynyl, aryl, heteroaryl, or heterocyclyl, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, C═O, C═S, —CN, —SiR¹⁶R¹⁷R¹⁸, or hydrogen;
each R³ and R⁴ is independently, alkyl, alkylene, alkenyl, alkenylene, alkynyl, aryl, heteroaryl, or heterocyclyl, each of which is independently substituted or unsubstituted, or —C(O)R¹⁹, —C(O)OR¹⁹, —C(O)NR¹⁹R²⁰, —SOR¹⁹, —SO₂R¹⁹, or hydrogen, or R³ and R⁴ together with the atom to which R³ and R⁴ are bound form a ring, wherein the ring is substituted or unsubstituted, or R³ is absent,
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹, or hydrogen or halogen;
each R¹⁹ and R²⁰ is independently alkyl, alkenyl, alkynyl, aryl, heteroaryl, or heterocyclyl, each of which is independently substituted or unsubstituted, C(O)R²³, —C(O)OR²³, —C(O)NR²³R²⁴, —OR²³, —SR²³, —NR²³R²⁴, —NR²³C(O)R²⁴, —OC(O)R²³, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen, or a pharmaceutically-acceptable salt thereof.

In some embodiments, the compound is of the formula:

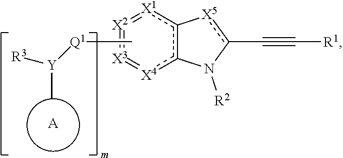

wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
   wherein at least one of X¹, X², X³, and X⁴ is a carbon atom connected to Q¹;
Q¹ is alkylene, alkenylene, or alkynylene, each of which is independently substituted or unsubstituted, or C═O, C═S, C═CR¹⁴R¹⁵, C═NR¹⁴, or a bond;
m is 1, 2, 3, or 4;
Y is N, O, or absent;
ring A is a cyclic group;
R¹ is alkyl, alkenyl, alkynyl, aryl, heteroaryl, or heterocyclyl, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, C═O, C═S, —CN, —SiR¹⁶R¹⁷R¹⁸, or hydrogen;
R³ is alkyl, alkylene, alkenyl, alkenylene, alkynyl, aryl, heteroaryl, or heterocyclyl, each of which is independently substituted or unsubstituted, or —C(O)R¹⁹, —C(O)OR¹⁹, —C(O)NR¹⁹R²⁰, —SOR¹⁹, —SO₂R¹⁹, or hydrogen, or R³ and A together with the atom to which R³ and A are bound form a ring, wherein the ring is substituted or unsubstituted, or R³ is absent,
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹, or hydrogen or halogen;
each R¹⁹ and R²⁰ is independently alkyl, alkenyl, alkynyl, aryl, heteroaryl, or heterocyclyl, each of which is independently substituted or unsubstituted, or —C(O)R²³, —C(O)OR²³, —C(O)NR²³R²⁴, —OR²³, —SR²³, —NR²³R²⁴, —NR²³C(O)R²⁴, —OC(O)R²³, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen, or a pharmaceutically-acceptable salt thereof.

In some embodiments, the pattern of dashed bonds is chosen to provide an aromatic system, for example, an indole, an indolene, a pyrrolopyridine, a pyrrolopyrimidine, or a pyrrolopyrazine.

In some embodiments, X¹ is CR⁵, CR⁵R⁶, or a carbon atom connected to Q¹. In some embodiments, X² is CR⁷, CR⁷R⁸, or a carbon atom connected to Q¹. In some embodiments, X³ is CR⁹, CR⁹R¹⁰, or a carbon atom connected to Q¹. In some embodiments, X⁴ is CR¹¹, CR¹¹R¹², or a carbon atom connected to Q¹. In some embodiments, X⁵ is CR¹³, N, or NR¹³. In some embodiments, X¹ is a carbon atom connected to Q¹. In some embodiments, X² is a carbon atom connected to Q¹. In some embodiments, X³ is a carbon atom connected to Q¹. In some embodiments, X⁴ is a carbon atom connected to Q¹. In some embodiments, X⁵ is N.

In some embodiments, Q¹ is a bond. In some embodiments, Q¹ is C₁-alkylene. In some embodiments, m is 1. In some embodiments, m is 2. In some embodiments, m is 3. In some embodiments, m is 4.

In some embodiments, R¹ is alkyl, alkenyl, each of which is unsubstituted or substituted, or —C(O)R¹⁶, —C(O)OR¹⁶, or —C(O)NR¹⁶R¹⁷. In some embodiments, R¹ is substituted alkyl. In some embodiments, R¹ is alkyl substituted with NR¹⁶R¹⁷.

In some embodiments, ring A is aryl, heteroaryl, or heterocyclyl, each of which is substituted or unsubstituted. In some embodiments, ring A is substituted aryl. In some embodiments, ring A is aryl substituted with fluoro-. In some embodiments, ring A is aryl substituted with chloro-. In some embodiments, ring A is substituted heteroaryl, In some embodiments, ring A is heteroaryl substituted with fluoro-. In some embodiments, ring A is heteroaryl substituted with chloro-. In some embodiments, ring A is substituted heterocyclyl. In some embodiments, ring A is heterocyclyl substituted with fluoro-. In some embodiments, A is heterocyclyl substituted with chloro-.

In some embodiments, ring A is piperidinyl, piperazinyl, tetahydropyranyl, morpholinyl, or pyrrolidinyl, each of which is independently substituted or unsubstituted. In some embodiments, ring A is piperidinyl, piperazinyl, tetahydropyranyl, morpholinyl, or pyrrolidinyl, each of which is independently substituted with at least halo-. In some embodiments, ring A is piperidinyl substituted with halo-. In some embodiments, ring A is methylpiperidinyl substituted with halo-. In some embodiments, ring A is 3-fluoro-1-methylpiperidinyl. In some embodiments, ring A is 3-fluoro-1-(2-hydroxy-3-methoxypropyl)piperidinyl. In some embodiments, ring A is tetrahydropyranyl substituted with at least halo-.

In some embodiments, each R¹⁶ and R¹⁷ is independently alkyl, alkenyl, aryl, heteroaryl, heterocyclyl, each of which is independently substituted or unsubstituted, or hydrogen. In some embodiments, R¹⁶ is hydrogen or alkyl. In some embodiments, R¹⁷ is aryl, heteroaryl, or heterocyclyl, each of which is independently substituted or unsubstituted. In some embodiments, R¹⁷ is substituted aryl. In some embodiments, R¹⁷ is substituted phenyl. In some embodiments, R¹⁷ is phenyl substituted with a sulfoxide group, carboxyl group, amide group, amino group, alkyl, alkoxy, hydroxy, or heterocyclyl, each of which is independently substituted or unsubstituted, or halo or cyano. In some embodiments, R¹⁷ is phenyl substituted with methoxy. In some embodiments, R¹⁷ is phenyl substituted with a substituted sulfoxide group. In some embodiments, R¹⁷ is phenyl substituted with a carboxyl group. In some embodiments, R¹⁷ is phenyl substituted with a substituted amide group.

In some embodiments, the compound is of the formula:

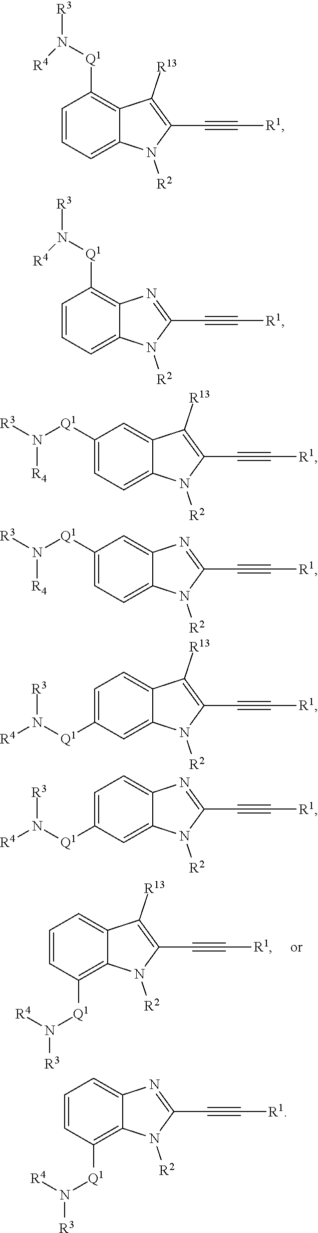

In some embodiments, Q¹ is alkylene, alkenylene, or alkynylene, each of which is independently substituted or unsubstituted, or C═O, C═S, C═CR¹⁴R¹⁵, C═NR¹⁴, or a bond. In some embodiments, Q¹ is alkylene, alkenylene, or alkynylene. In some embodiments, Q¹ is C₁-alkylene or a bond. In some embodiments, Q¹ is C₁-alkylene. In some embodiments, Q¹ is a bond.

In some embodiments, Y is N. In some embodiments, Y is O. In some embodiments, Y is absent.

In some embodiments, R² is hydrogen or alkyl. In some embodiments, R² is alkyl. In some embodiments, R² is substituted C₁-C₅-alkyl. In some embodiments, R² is trifluoroethyl. In some embodiments, R² is cycloalkyl. In some embodiments, R² is cyclopropyl.

In some embodiments, R¹³ is alkyl, alkenyl, hydrogen, or halogen. In some embodiments, R¹³ is hydrogen.

In some embodiments, R² is C₁-C₅-alkyl, and R¹³ is C₁-C₅-alkyl. In some embodiments, R² is C₁-C₅-alkyl, and R¹³ is hydrogen. In some embodiments, R² is substituted C₁-C₅-alkylene. In some embodiments, R² is methyl, ethyl, propyl, iso-propyl, butyl, or tert-butyl, each of which is substituted or unsubstituted. In some embodiments, R¹³ is methyl, ethyl, propyl, iso-propyl, butyl or tert-butyl. In some embodiments, R² is hydrogen, and R¹³ is hydrogen. In some embodiments, R² is trifluoroethyl, and R¹³ is hydrogen.

In some embodiments, the compound is of the formula:

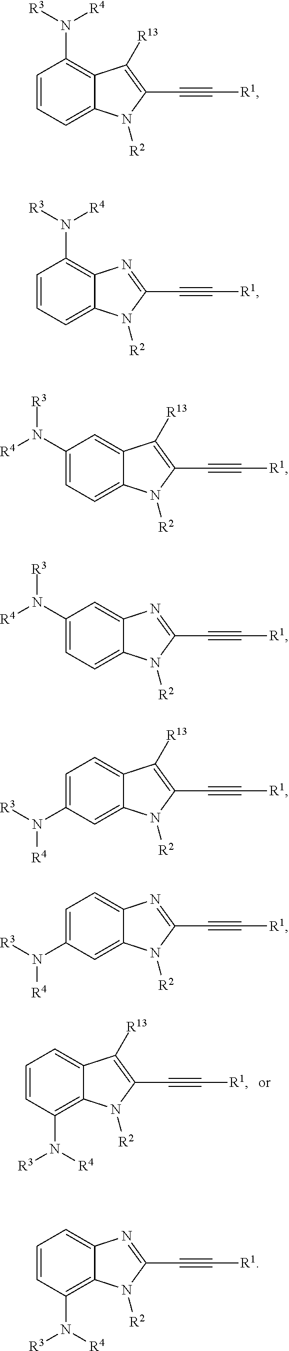

In some embodiments, the compound is of the formula:

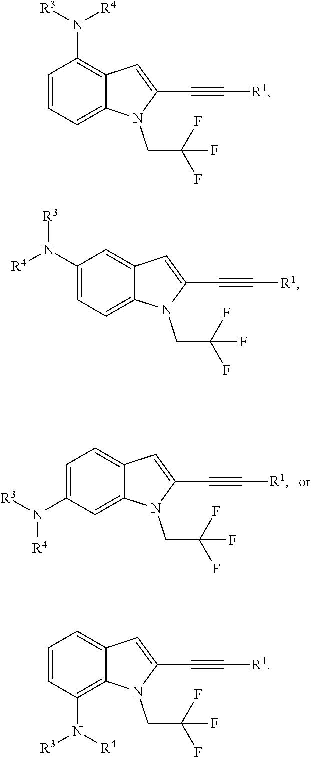

In some embodiments, R³ is H, and R⁴ is alkyl, alkylene, alkenyl, alkenylene, alkynyl, aryl, heteroaryl, or heterocyclyl, each of which is independently substituted or unsubstituted, or —C(O)R¹⁹, —C(O)OR¹⁹, —C(O)NR¹⁹R²⁰, —SOR¹⁹, —SO₂R¹⁹. In some embodiments, each R³ and R⁴ is independently substituted or unsubstituted C₁-C₆-alkylene. In some embodiments, R³ is H, and R⁴ is substituted or unsubstituted C₁-C₄ alkylene. In some embodiments, R³ is H, and R⁴ is substituted or unsubstituted heterocyclyl. In some embodiments, R³ is H, and R⁴ is substituted or unsubstituted piperidinyl. In some embodiments, R³ is H, and R⁴ is substituted or unsubstituted cycloalkyl. In some embodiments, R³ is H, and R⁴ is cycloalkyl substituted with an amino group. In some embodiments, R³ is H, and R⁴ is substituted or unsubstituted cyclobutyl. In some embodiments, R³ is H, and R⁴ is cyclobutyl substituted with an amino group. In some embodiments, R³ is H, and R⁴ is substituted or unsubstituted cyclohexyl. In some embodiments, R³ is H, and R⁴ is cyclohexyl substituted with an amino group.

In some embodiments, the compound is of the formula:

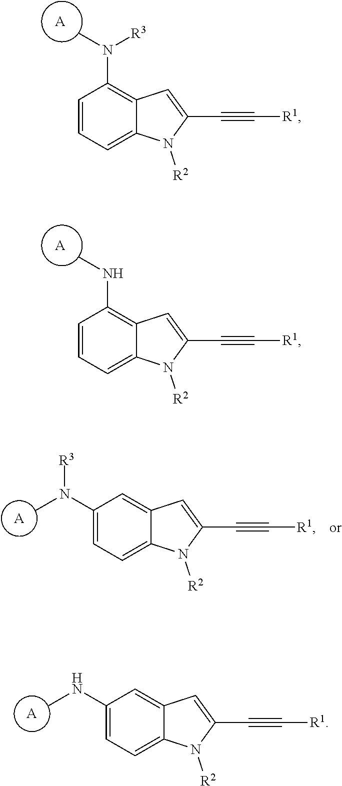

In some embodiments, the compound is of the formula:

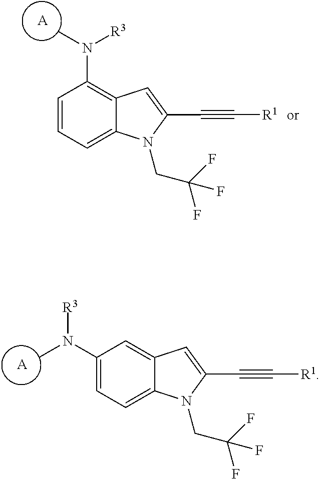

R¹ can be a group substituted with one or more substituents selected from a hydroxyl group, sulfhydryl group, halogens, amino group, nitro group, nitroso group, cyano group, azido group, sulfoxide group, sulfone group, sulfonamide group, carboxyl group, carboxaldehyde group, imine group, alkyl group, halo-alkyl group, cyclic alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, urethane group, and ester group. In some embodiments, R¹ is alkyl, alkenyl, —C(O)R¹⁶, —C(O)OR¹⁶, or —C(O)NR¹⁶R¹⁷.

In some embodiments, R¹ is substituted or unsubstituted C₁-C₃ alkyl. In some embodiments, R¹ is C₁-C₃-alkyl substituted with an amine group. In some embodiments, R¹ is C₁-alkyl substituted with NR¹⁶R¹⁷. In some embodiments, each R¹⁶ and R¹⁷ is independently aryl, heteroaryl, or heterocyclyl, each of which is independently substituted or unsubstituted, or hydrogen. In some embodiments, R¹⁶ is H, and R¹⁷ is substituted aryl. In some embodiments, R¹⁶ is H, and R¹⁷ is substituted phenyl. In some embodiments, R¹⁶ is H, and R¹⁷ is phenyl substituted with alkyl, alkoxy, halo, sulfonamide, a sulfone, or a carboxy group. In some embodiments, R¹⁶ is H, and R¹⁷ is substituted heteroaryl. In some embodiments, R¹⁶ is H, and R¹⁷ is substituted heterocyclyl.

In some embodiments, Q¹ is alkylene, alkenylene, or alkynylene, each of which is independently substituted or unsubstituted, or C═O, C═S, C═CR¹⁴R¹⁵, C═NR¹⁴, or a bond. In some embodiments, Q¹ is alkylene, alkenylene, or alkynylene. In some embodiments, Q¹ is C₁-alkylene. In some embodiments, each R¹⁶ and R¹⁷ is independently alkyl, alkenyl, aryl, heteroaryl, heterocyclyl, or hydrogen. In some embodiments, Q¹ is C₁-alkylene, R¹⁶ is aryl, and R¹⁷ is alkyl. In some embodiments, Q¹ is C₁-alkylene, R¹⁶ is aryl, and R¹⁷ is hydrogen. In some embodiments, Q¹ is C₁-alkylene, R¹⁶ is heteroaryl, and R¹⁷ is alkyl. In some embodiments, Q¹ is C₁-alkylene, R¹⁶ is heteroaryl, and R¹⁷ is hydrogen. In some embodiments, Q¹ is C₁-alkylene, R¹⁶ is substituted heteroaryl, and R¹⁷ is hydrogen. In some embodiments, Q¹ is C₁-alkylene, R¹⁶ is substituted alkyl, and R¹⁷ is hydrogen. In some embodiments, R¹⁷ is aryl, heteroaryl, or heterocyclyl, each of which is independently substituted or unsubstituted with halogen, alkyl, or hydroxyl. In some embodiments, R¹⁶ is hydrogen, and R¹⁷ is aryl or heteroaryl, substituted or unsubstituted with halogen or alkyl. In some embodiments, R¹⁶ is alkyl, and R¹⁷ is heteroaryl substituted with halogen or alkyl. In some embodiments, R¹⁷ is aryl, heteroaryl, or heterocyclyl, each of which is independently substituted or unsubstituted with alkyl. In some embodiments, R¹⁷ is aryl or heteroaryl, each of which is independently substituted with alkyl, wherein the alkyl is optionally substituted with fluorine, chlorine, bromine, iodine, or cyano.

In some embodiments, R² is alkyl, and R¹³ is alkyl, each of which is substituted or substituted. In some embodiments, R² is hydrogen, and R¹³ is unsubstituted or substituted alkyl. In some embodiments, R² is methyl, ethyl, propyl, iso-propyl, butyl, or tert-butyl, each of which is substituted or unsubstituted. In some embodiments, R¹³ is methyl, ethyl, propyl, iso-propyl, butyl or tert-butyl. In some embodiments, R² is hydrogen, and R¹³ is hydrogen. In some embodiments, R² is hydrogen, and R¹³ is alkyl. In some embodiments, R² is trifluoroethyl, and R¹³ is hydrogen.

In some embodiments, R³ is alkyl, alkenyl, alkynyl, aryl, heteroaryl, or heterocyclyl, each of which is independently substituted or unsubstituted, or —C(O)R¹⁹, —C(O)OR¹⁹, or hydrogen, and R⁴ is alkyl, alkenyl, alkynyl, aryl, heteroaryl, or heterocyclyl, each of which is independently substituted or unsubstituted, or —C(O)R¹⁹, —C(O)OR¹⁹, or hydrogen. In some embodiments, R³ is alkyl, alkenyl, alkynyl, aryl, heteroaryl, or heterocyclyl, each of which is independently substituted or unsubstituted, or hydrogen. In some embodiments, R³ is substituted alkyl. In some embodiments, R³ is H.

In some embodiments, R³ is H, and R⁴ is unsubstituted or substituted alkyl. In some embodiments, R³ is H, and R⁴ is unsubstituted or substituted cycloalkyl. In some embodiments, R³ is H, and R⁴ is substituted cyclohexyl. In some embodiments, R³ is H, and R⁴ is substituted cyclobutyl.

In some embodiments, at least one of R³ and R⁴ is alkyl, alkylene, alkenyl, alkenylene, alkynyl, aryl, heteroaryl, or heterocyclyl, each of which is substituted at least with halo-. In some embodiments, R³ is hydrogen and R⁴ is a ring A. In some embodiments, R⁴ or ring A is cycloalkyl, aryl, heteroaryl, or heterocyclyl, each of which is independently substituted or unsubstituted. In some embodiments, R⁴ or ring A is substituted or unsubstituted aryl. In some embodiments, R⁴ or ring A is substituted or unsubstituted phenyl. In some embodiments, R⁴ or ring A is substituted or unsubstituted cycloalkyl. In some embodiments, R⁴ or ring A is substituted or unsubstituted cyclopropyl. In some embodiments, R⁴ or ring A is substituted cyclopropyl. In some embodiments, R⁴ or ring A is substituted cyclohexyl. In some embodiments, R⁴ or ring A is cyclohexyl substituted with an amino group.

In some embodiments, R³ is H, and R⁴ or ring A is unsubstituted or substituted heterocyclyl. In some embodiments, R⁴ or ring A is heterocyclyl. In some embodiments, R⁴ or ring A is piperidinyl, piperazinyl, tetahydropyranyl, morpholinyl, or pyrrolidinyl, each of which is independently substituted or unsubstituted. In some embodiments, R³ is H, and R⁴ or ring A is substituted piperidinyl. In some embodiments, R³ is H, and R⁴ or ring A is piperidine substituted with alkyl, carboxy, heterocyclyl, or an amide group. In some embodiments, R³ is H, and R⁴ or ring A is unsubstituted or substituted methyl piperidinyl. In some embodiments, R³ is H, and R⁴ or ring A is 3-fluoro-1-methylpiperidinyl. In some embodiments, R³ is H, and R⁴ or ring A is piperidinyl substituted with methoxypropanol. In some embodiments, R³ is H, and R⁴ or ring A is 3-fluoro-1-(2-hydroxy-3-methoxypropyl)piperidinyl. In some embodiments, R³ is H, and R⁴ or ring A is unsubstituted or substituted tetrahydropyranyl. In some embodiments, R³ is H, and R⁴ or ring A is unsubstituted tetrahydropyranyl. In some embodiments, R³ is H, and R⁴ or ring A is tetrahydropyranyl substituted with alkyl. In some embodiments, R³ is H, and R⁴ or ring A is tetrahydrothiopyran-1,1-diooxide.

In some embodiments, R⁴ or ring A is cycloalkyl, aryl, heteroaryl, or heterocyclyl, each of which is substituted at least with halo-. In some embodiments, R⁴ or ring A is C₄-C₆-cycloalkyl substituted with at least halo-. In some embodiments, R⁴ or ring A is cyclohexyl substituted with at least halo-. In some embodiments, R⁴ or ring A is aryl substituted with at least halo-. In some embodiments, R⁴ or ring A is phenyl substituted with at least halo-. In some embodiments, R⁴ or ring A is aryl substituted with fluoro-. In some embodiments, R⁴ or ring A is phenyl substituted with fluoro-. In some embodiments, R⁴ or ring A is aryl substituted with chloro-. In some embodiments, R⁴ or ring A is phenyl substituted with chloro-. In some embodiments, R⁴ or ring A is heteroaryl substituted with at least halo-. In some embodiments, R⁴ or ring A is heteroaryl substituted with fluoro-. In some embodiments, R⁴ or ring A is heteroaryl substituted with chloro-. In some embodiments, R⁴ or ring A is C₄-C₆-heterocyclyl substituted with at least halo-. In some embodiments, R⁴ or ring A is heterocyclyl substituted with fluoro-. In some embodiments, R⁴ or ring A is heterocyclyl substituted with chloro-.

In some embodiments, R⁴ or ring A is piperidinyl, piperazinyl, tetahydropyranyl, morpholinyl, or pyrrolidinyl, each of which is independently substituted with at least halo-. In some embodiments, R⁴ or ring A is piperidinyl substituted with halo-. In some embodiments, R⁴ or ring A is methylpiperidinyl substituted with halo-. In some embodiments, R⁴ or ring A is 3-fluoro-1-methylpiperidinyl. In some embodiments, R⁴ or ring A is 3-fluoro-1-(2-hydroxy-3-methoxypropyl)piperidinyl. In some embodiments, R⁴ or ring A is tetrahydropyranyl substituted with at least halo-.

In some embodiments, R⁴ or Ring A is a ring that is:

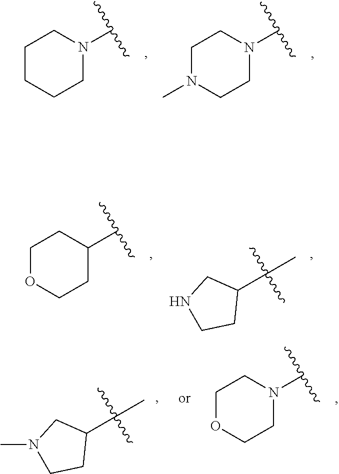

wherein the ring is substituted or unsubstituted. In some embodiments, the ring is substituted with halo-. In some embodiments, the ring is substituted with fluoro. In some embodiments, R³ is H, and R⁴ is a ring that is

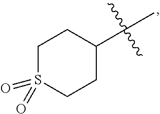 [SMILES: *C1CCS(=O)(=O)CC1]

wherein the ring is substituted or unsubstituted. In some embodiments, the ring is substituted with halo-. In some embodiments, the ring is substituted with fluoro. In some embodiments, R³ is H, and R⁴ is a ring that is

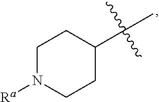

wherein the ring is substituted or unsubstituted. In some embodiments, R^{a} is alkylene. In some embodiments, R^{a} is methyl. In some embodiments, the ring is substituted with halo. In some embodiments, the ring is substituted with fluoro. In some embodiments, R³ is H, and R⁴ is a ring that is

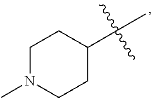 [SMILES: *C1CCN(C)CC1]

wherein the ring is substituted or unsubstituted. In some embodiments, the ring is substituted with halo. In some embodiments, the ring is substituted with fluoro. In some embodiments, R³ is H, and R⁴ is a ring that is

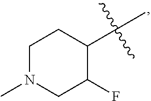 [SMILES: *C1CCN(C)CC1F]

wherein the ring is substituted or unsubstituted.

In some embodiments, the R⁴ or ring A is substituted with one or more substituents selected from a hydroxyl group, sulfhydryl group, halogens, amino group, nitro group, nitroso group, cyano group, azido group, sulfoxide group, sulfone group, sulfonamide group, carboxyl group, carboxaldehyde group, imine group, alkyl group, halo-alkyl group, cyclic alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, urethane group, and ester group.

In some embodiments, R³ and R⁴ together with the nitrogen atom to which R³ and R⁴ are bound form a ring, wherein the ring is substituted or unsubstituted. In some embodiments, R³ and R⁴ together with the nitrogen atom to which R³ and R⁴ are bound form a substituted heterocycle. In some embodiments, R³ and R⁴ together with the nitrogen atom to which R³ and R⁴ are bound form a heterocycle substituted with a hydroxyl group, halogen, amino group, or alkyl group. In some embodiments, R³ and R⁴ together with the nitrogen atom to which R³ and R⁴ are bound form a heterocycle, wherein the heterocycle is substituted by a substituted or unsubstituted heterocycle.

In some embodiments, R³ and R⁴ together with the nitrogen atom to which R³ and R⁴ are bound form a ring of a following formula:

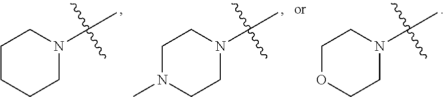

In some embodiments, the compound is of the formula:

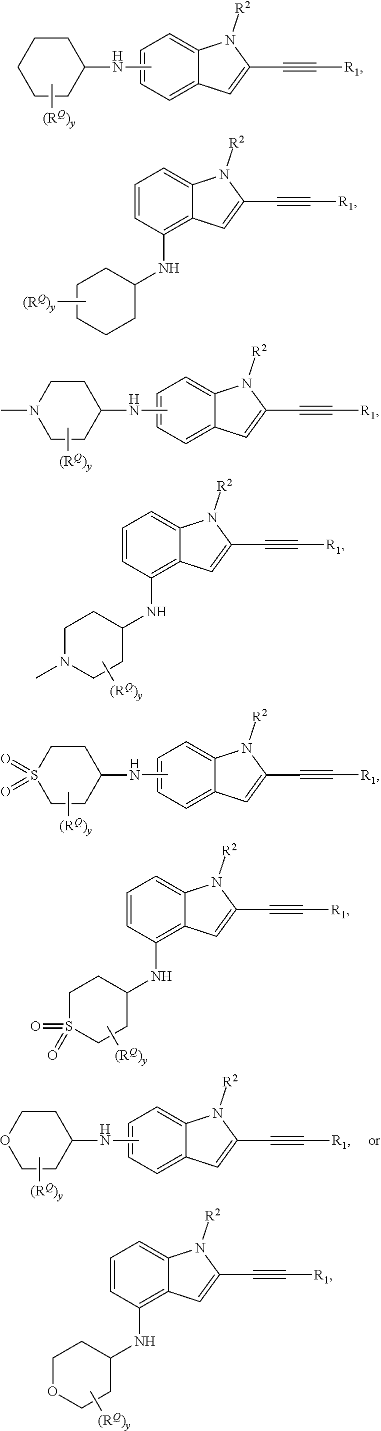

wherein:
R¹ is alkyl, alkenyl, alkynyl, aryl, heteroaryl, or heterocyclyl, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, C═O, C═S, —CN, —SiR¹⁶R¹⁷R¹⁸, or hydrogen;
each R^{Q} is independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹, or hydrogen or halogen;
y is 0, 1, 2, 3, or 4;
each R¹⁶, R¹⁷, and R¹⁸ is independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹, or hydrogen or halogen;
each R¹⁹ and R²⁰ is independently alkyl, alkenyl, alkynyl, aryl, heteroaryl, or heterocyclyl, each of which is independently substituted or unsubstituted, or C(O)R²³, —C(O)OR²³, —C(O)NR²³R²⁴, —OR²³, —SR²³, —NR²³R²⁴, —NR²³C(O)R²⁴, —OC(O)R²³, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, R¹ is alkyl, alkenyl, alkynyl, alkoxy, aryl, heteroaryl, or heterocyclyl, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, or hydrogen. In some embodiments, R¹ is alkyl, alkylene, alkoxy, —NR²¹R²², or aryl, each of which is independently substituted or unsubstituted; halogen or hydrogen.

In some embodiments, R¹ is substituted C₁-C₃-alkyl. In some embodiments, R¹ is C₁-C₃-alkyl substituted with NR¹⁶R¹⁷. In some embodiments, R¹ is methyl substituted with NR¹⁶R¹⁷, wherein each R¹⁶ and R¹⁷ is independently alkyl, cycloalkyl, aryl, heteroaryl, heterocyclyl, alkoxy, carboxyl group, amino group, acyl group, acyloxy group, or an amide group, any of which is unsubstituted or substituted, or hydrogen. In some embodiments, R¹ is methyl substituted with NR¹⁶R¹⁷, wherein R¹⁶ is hydrogen, and R¹⁷ is a substituted carboxyl group. In some embodiments, R¹ is methyl substituted with NR¹⁶R¹⁷, wherein R¹⁶ is hydrogen, and R¹⁷ is substituted aryl. In some embodiments, R¹ is methyl substituted with NR¹⁶R¹⁷, wherein R¹⁶ is hydrogen, and R¹⁷ is substituted phenyl. In some embodiments, R¹ is methyl substituted with NR¹⁶R¹⁷, wherein R¹⁶ is hydrogen, and R¹⁷ is phenyl substituted with a sulfoxide group, carboxyl group, amide group, amino group, alkyl, alkoxy, hydroxy, or heterocyclyl, each of which is independently substituted or unsubstituted, or halo or cyano. In some embodiments, R¹⁷ is phenyl substituted with methoxy. In some embodiments, R¹⁷ is phenyl substituted with a substituted sulfoxide group. In some embodiments, R¹⁷ is phenyl substituted with a carboxyl group. In some embodiments, R¹⁷ is a substituted amide group. In some embodiments, R¹⁷ is substituted with methoxy and sulfonamide.

In some embodiments, R² is hydrogen or alkyl. In some embodiments, R² is substituted C₁-C₅-alkylene. In some embodiments, R² is trifluoroethyl. In some embodiments, R¹³ is alkyl, alkenyl, hydrogen, or halogen. In some embodiments, R² is alkyl, and R¹³ is alkyl. In some embodiments, R² is hydrogen, and R¹³ is alkyl. In some embodiments, R² is methyl, ethyl, propyl, iso-propyl, butyl, or tert-butyl. In some embodiments, R¹³ is methyl, ethyl, propyl, iso-propyl, butyl or tert-butyl. In some embodiments, R² is hydrogen, and R¹³ is hydrogen.

In some embodiments, the compound is of the formula:

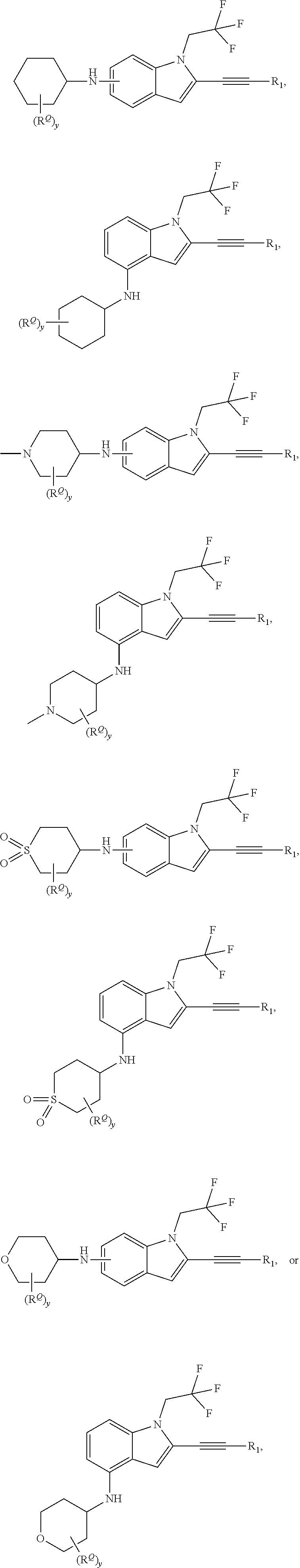

or a pharmaceutically-acceptable salt thereof, wherein the variables are as defined above.

In some embodiments, each R^{Q} is independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NRC(O)R², —OC(O)R²¹, or hydrogen or halogen.

In some embodiments, y is 1. In some embodiments, y is 2. In some embodiments, y is 3. In some embodiments, y is 4.

In some embodiments, R¹ is alkyl, alkenyl, alkynyl, alkoxy, aryl, heteroaryl, or heterocyclyl, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, or hydrogen. In some embodiments, R¹ is alkyl, alkylene, alkoxy, —NR²¹R²², or aryl, each of which is independently substituted or unsubstituted; halo or hydrogen.

In some embodiments, R¹ is substituted alkyl. In some embodiments, R¹ is substituted C₁-C₃-alkyl. In some embodiments, R¹ is alkyl substituted with NR¹⁶R¹⁷. In some embodiments, R¹ is C₁-C₃-alkyl substituted with NR¹⁶R¹⁷. In some embodiments, R¹ is methyl substituted with NR¹⁶R¹⁷, wherein each R¹⁶ and R¹⁷ is independently alkyl, cycloalkyl, aryl, heteroaryl, heterocyclyl, alkoxy, carboxyl group, amino group, acyl group, acyloxy group, or an amide group, any of which is unsubstituted or substituted, or hydrogen. In some embodiments, R¹ is methyl substituted with NR¹⁶R¹⁷, wherein R¹⁶ is hydrogen, and R¹⁷ is a substituted carboxyl group. In some embodiments, R¹ is methyl substituted with NR¹⁶R¹⁷, wherein R¹⁶ is hydrogen, and R¹⁷ is a substituted aryl group. In some embodiments, R¹ is methyl substituted with NR¹⁶R¹⁷, wherein R¹⁶ is hydrogen, and R¹⁷ is a substituted phenyl group.

In some embodiments, R¹⁶ is alkyl, alkenyl, aryl, heteroaryl, heterocyclyl, or hydrogen, and R¹⁷ is aryl, heteroaryl, or heterocyclyl. In some embodiments, R¹⁶ is hydrogen, and R¹⁷ is phenyl, indolyl, piperidinyl, imidazolyl, thiazolyl, morpholinyl, pyrrolyl, or pyridinyl, each of which is substituted or unsubstituted.

In some embodiments, the compound is of the formula:

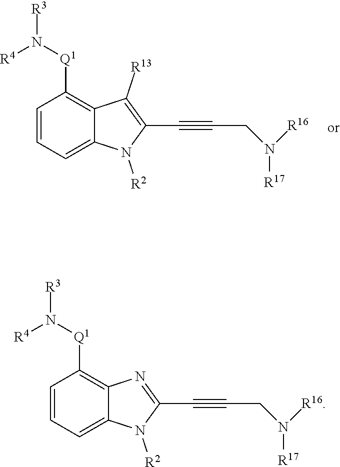

In some embodiments, the compound is of the formula:

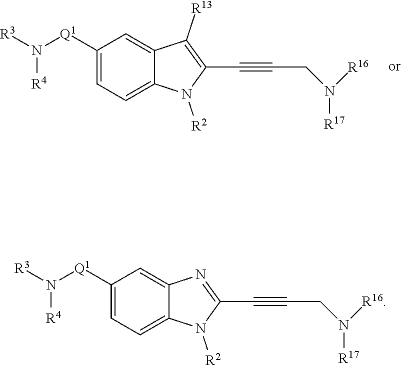

In some embodiments, the compound is of the formula:

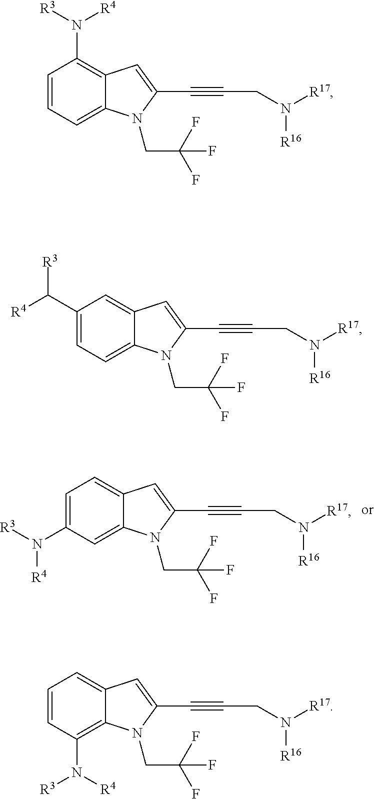

In some embodiments, each R¹⁶ and R¹⁷ is independently alkyl, alkenyl, aryl, heteroaryl, heterocyclyl, or hydrogen. In some embodiments, R¹⁶ is aryl, and R¹⁷ is alkyl. In some embodiments, R¹⁶ is aryl, and R¹⁷ is hydrogen. In some embodiments, R¹⁶ is heteroaryl, and R¹⁷ is alkyl. In some embodiments, R¹⁶ is heteroaryl, and R¹⁷ is hydrogen. In some embodiments, R¹⁶ is substituted heteroaryl, and R¹⁷ is hydrogen. In some embodiments, R¹⁶ is substituted alkyl, and R¹⁷ is hydrogen. In some embodiments, R¹⁷ is aryl, heteroaryl, or heterocyclyl, each of which is independently substituted or unsubstituted with halogen, alkyl, or hydroxyl. In some embodiments, R¹⁶ is hydrogen, and R¹⁷ is aryl or heteroaryl, substituted or unsubstituted with halogen or alkyl. In some embodiments, R¹⁶ is alkyl, and R¹⁷ is heteroaryl substituted with halogen or alkyl. In some embodiments, R¹⁶ is hydrogen. In some embodiments, R¹⁷ is aryl, heteroaryl, or heterocyclyl, each of which is independently substituted or unsubstituted with alkyl. In some embodiments, R¹⁷ is aryl or heteroaryl, each of which is independently substituted with alkyl, wherein the alkyl is optionally substituted with fluorine, chlorine, bromine, iodine, or cyano. In some embodiments, R¹⁶ is alkyl, alkenyl, aryl, heteroaryl, heterocyclyl, or hydrogen, and R¹⁷ is aryl, heteroaryl, or heterocyclyl. In some embodiments, R¹⁶ is hydrogen, and R¹⁷ is phenyl, indolyl, piperidinyl, imidazolyl, thiazolyl, morpholinyl, pyrrolyl, or pyridinyl, each of which is substituted or unsubstituted. In some embodiments, R¹⁶ is hydrogen, and R¹⁷ is substituted phenyl. In some embodiments, R¹⁶ is hydrogen, and R¹⁷ is phenyl substituted with a sulfoxide group, carboxyl group, amide group, amino group, alkyl, alkoxy, hydroxy, or heterocyclyl, each of which is independently substituted or unsubstituted, or halo or cyano. In some embodiments, R¹⁷ is phenyl substituted with methoxy. In some embodiments, R¹⁷ is phenyl substituted with a substituted sulfoxide group. In some embodiments, R¹⁷ is phenyl substituted with a carboxyl group. In some embodiments, R¹⁷ is a substituted amide group. In some embodiments, R¹⁷ is substituted with methoxy and sulfonamide.

In some embodiments, each R³ and R⁴ is independently unsubstituted or substituted alkyl. In some embodiments, R³ is hydrogen and R⁴ is alkyl, alkenyl, alkynyl, aryl, heteroaryl, or heterocyclyl, each of which is independently substituted or unsubstituted, —C(O)R¹⁹, or —C(O)OR¹⁹. In some embodiments, R³ is hydrogen, and R⁴ is alkyl, aryl, heteroaryl, or heterocyclyl, each of which is independently substituted or unsubstituted. In some embodiments, R³ is H, and R⁴ is substituted heterocyclyl. In some embodiments, R³ is H, and R⁴ is substituted or unsubstituted C₄-C₆-heterocyclyl. In some embodiments, R³ is H, and R⁴ is substituted alkyl. In some embodiments, R³ is H, and R⁴ is substituted C₁-C₆-alkyl. In some embodiments, R³ is H, and R⁴ is substituted or unsubstituted cycloalkyl. In some embodiments, R³ is H, and R⁴ is substituted or unsubstituted C₄-C₆-cycloalkyl. In some embodiments, R³ is H, and R⁴ is C₄-C₆-cycloalkyl substituted with an amino group.

In some embodiments, the compound is of the formula:

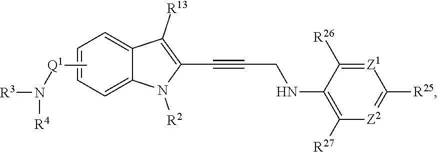

wherein:
Q¹ is alkylene, alkenylene, or alkynylene, each of which is independently substituted or unsubstituted, or C═O, C═S, C═CR¹⁴R¹⁵, C═NR¹⁴, or a bond;
R¹ is alkyl, alkenyl, alkynyl, aryl, heteroaryl, or heterocyclyl, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, C═O, C═S, —CN, —SiR¹⁶R¹⁷R¹⁸, or hydrogen;
each R³ and R⁴ is independently, alkyl, alkylene, alkenyl, alkenylene, alkynyl, aryl, heteroaryl, or heterocyclyl, each of which is independently substituted or unsubstituted, or —C(O)R¹⁹, —C(O)OR¹⁹, —C(O)NR¹⁹R²⁰, —SOR¹⁹, —SO₂R¹⁹, or hydrogen, or R³ and R⁴ together with the nitrogen atom to which R³ and R⁴ are bound form a ring, wherein the ring is substituted or unsubstituted, or R³ is absent;
each Z¹ and Z² is independently CR²⁸, CR²⁹, or N;
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹, or hydrogen or halogen;
each R¹⁹ and R²⁰ is independently alkyl, alkenyl, alkynyl, aryl, heteroaryl, or heterocyclyl, each of which is independently substituted or unsubstituted, or —C(O)R²³, —C(O)OR²³, —C(O)NR²³R²⁴, —OR²³, —SR²³, —NR²³R²⁴, —NR²³C(O)R²⁴, —OC(O)R²³, or hydrogen or halogen;
each R²¹ and R²² is independently alkyl, alkenyl, alkynyl, aryl, heteroaryl, or heterocyclyl, each of which is independently substituted or unsubstituted, or hydrogen;
each R²³ and R²⁴ is independently alkyl, alkenyl, alkynyl, aryl, heteroaryl, or heterocyclyl, each of which is independently substituted or unsubstituted, or hydrogen; and
each R²⁵, R²⁶, R²⁷, R²⁸, and R²⁹ is independently hydrogen or a substituent selected from a hydroxyl group, sulfhydryl group, halogen, amino group, nitro group, nitroso group, cyano group, azido group, sulfoxide group, sulfone group, sulfonamide group, carboxyl group, carboxaldehyde group, imine group, alkyl group, halo-alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, ureido group, epoxy group, and ester group.
   or a pharmaceutically-acceptable salt thereof.

In some embodiments, Z¹ is N. In some embodiments, Z¹ and Z² are N. In some embodiments, each R²⁵ and R²⁶ is independently a halogen. In some embodiments, R²⁵ is

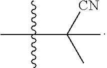 [SMILES: *C(C)(C)C#N]

In some embodiments, R²⁵ is a substituted sulfone group. In some embodiments, R²⁵ is a sulfone group substituted with alkyl. In some embodiments, R²⁵ is a methanesulfonyl group. In some embodiments, R²⁵ is a sulfone group substituted with an amino group. In some embodiments, R²⁵ is a sulfonamide. In some embodiments, R²⁵ is a carboxy group. In some embodiments, R²⁵ is a methoxycarbonyl group.

In some embodiments, the compound is of the formula:

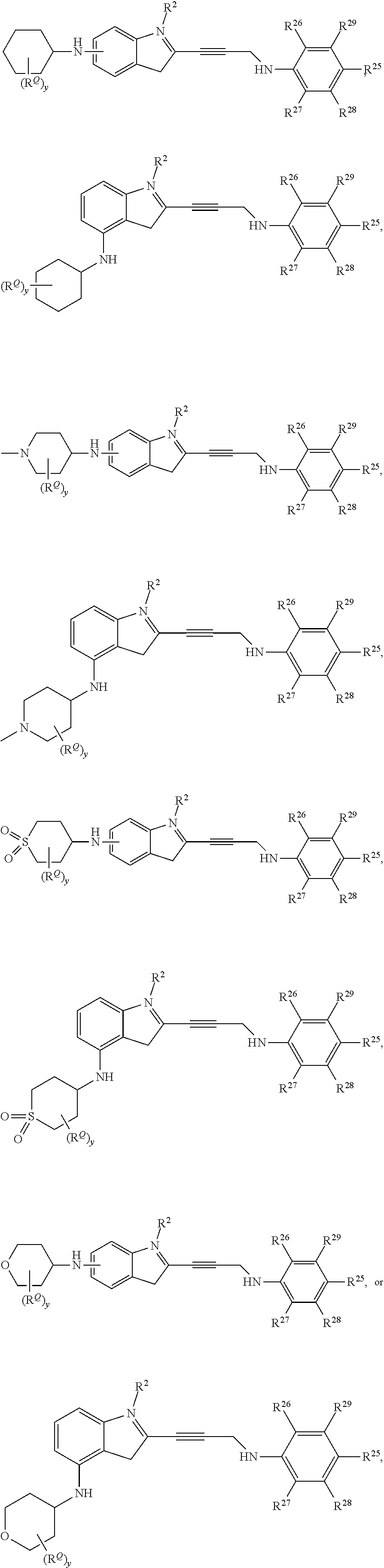

wherein:
R² is alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹, or hydrogen or halogen;
each R^{Q} is independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹;
y is 0, 1, 2, 3, or 4;
each R²¹ and R²² is independently alkyl, alkenyl, alkynyl, aryl, heteroaryl, or heterocyclyl, each of which is independently substituted or unsubstituted, or hydrogen; and
each R²⁵, R²⁶, R, R²⁸, and R²⁹ is independently hydrogen or a substituent selected from a hydroxyl group, sulfhydryl group, halogen, amino group, nitro group, nitroso group, cyano group, azido group, sulfoxide group, sulfone group, sulfonamide group, carboxyl group, carboxaldehyde group, imine group, alkyl group, halo-alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, ureido group, epoxy group, and ester group.
   or a pharmaceutically-acceptable salt thereof.

In some embodiments, the compound is of the formula:

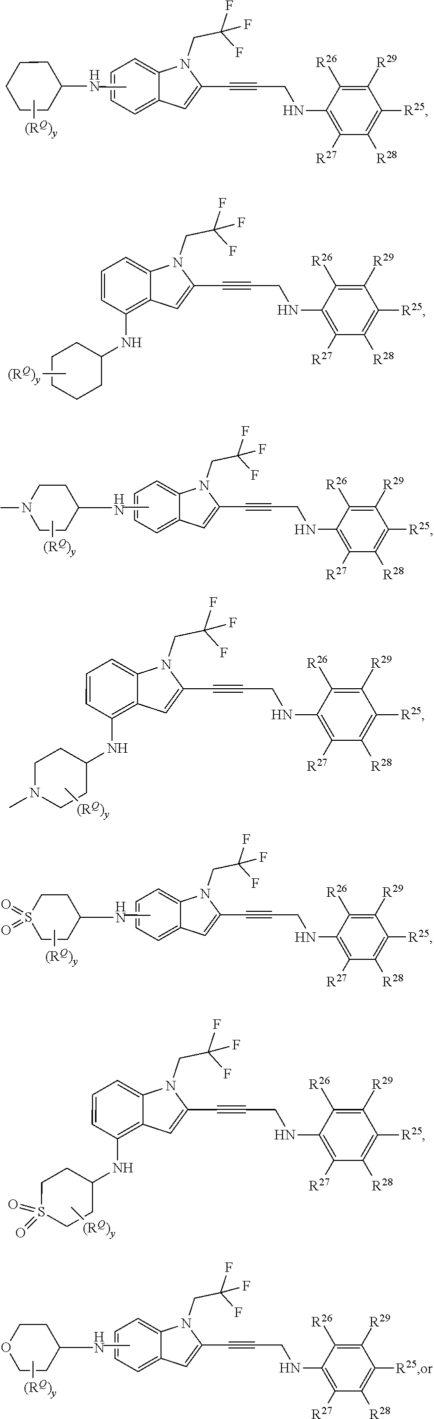

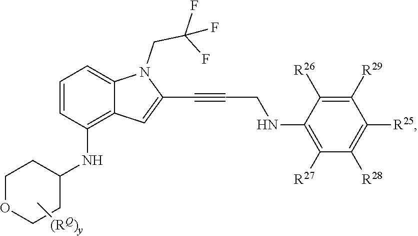

In some embodiments, R²⁵ is a substituted sulfone group. In some embodiments, R²⁵ is a sulfone group substituted with alkyl. In some embodiments, R²⁵ is a methanesulfonyl group. In some embodiments, R²⁵ is a sulfone group substituted with an amino group. In some embodiments, R²⁵ is a sulfonamide. In some embodiments, R²⁵ is a carboxy group. In some embodiments, R²⁵ is a methoxycarbonyl group.

In some embodiments, the compound is of the formula:

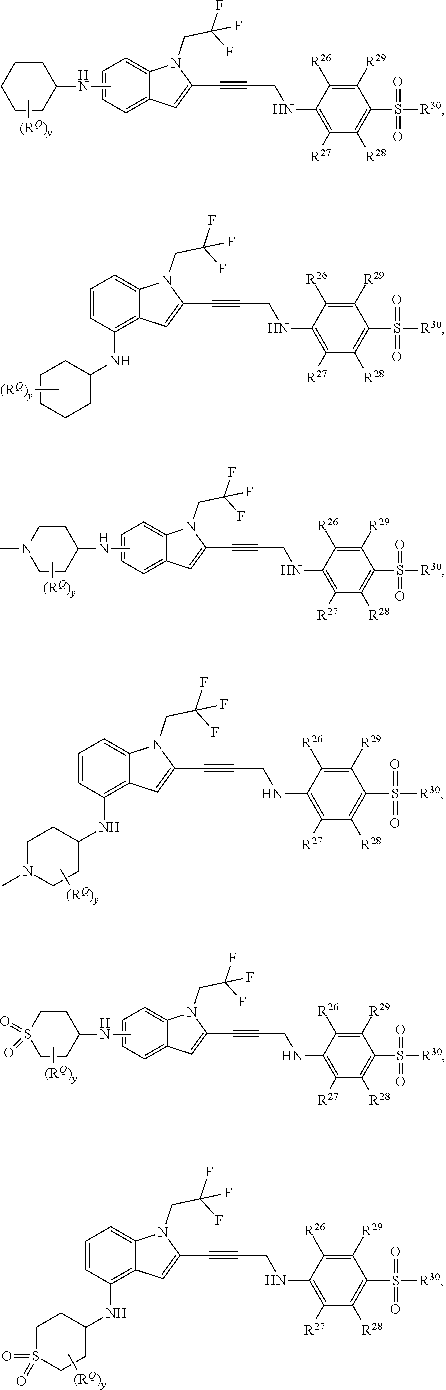

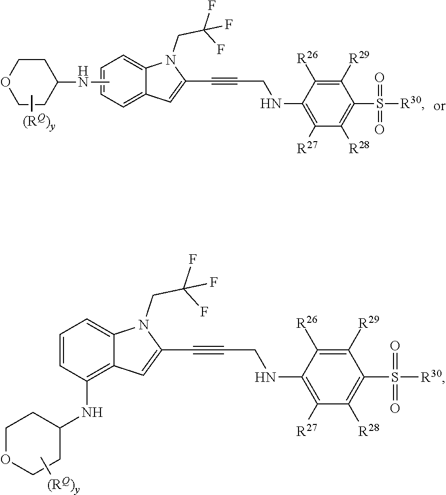

wherein:
each R^{Q} is independently alkyl, alkenyl, alkynyl, aryl, heteroaryl, or heterocyclyl, each of which is independently substituted or unsubstituted, or —C(O)R²¹, —C(O)OR²¹, —C(O)NR²¹R²², —OR²¹, —SR²¹, —NR²¹R²², —NR²¹C(O)R²², —OC(O)R²¹;
y is 0, 1, 2, 3, or 4;
each R²¹ and R²² is independently alkyl, alkenyl, alkynyl, aryl, heteroaryl, or heterocyclyl, each of which is independently substituted or unsubstituted, or hydrogen;
each R²⁶, R²⁷, R²⁸, and R²⁹ is independently hydrogen or a substituent selected from a hydroxyl group, sulfhydryl group, halogen, amino group, nitro group, nitroso group, cyano group, azido group, sulfoxide group, sulfone group, sulfonamide group, carboxyl group, carboxaldehyde group, imine group, alkyl group, halo-alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, ureido group, epoxy group, and ester group; and
R³⁰ is alkyl or an amino group, each of which is substituted or unsubstituted,
   or a pharmaceutically-acceptable salt thereof.

In some embodiments, R³⁰ is methyl. In some embodiments, R³⁰ is NH₂. In some embodiments, R³⁰ is NHMe. In some embodiments, R³⁰ is NMe₂.

In some embodiments, the compound is of the formula:

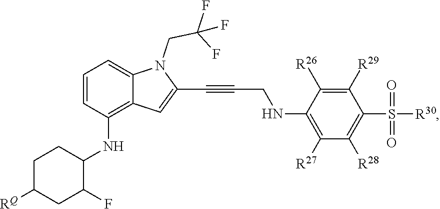

wherein R³⁰ is alkyl or an amino group, each of which is unsubstituted or substituted. In some embodiments, R³⁰ is methyl.

Non-limiting examples of compounds of the current disclosure include the following:

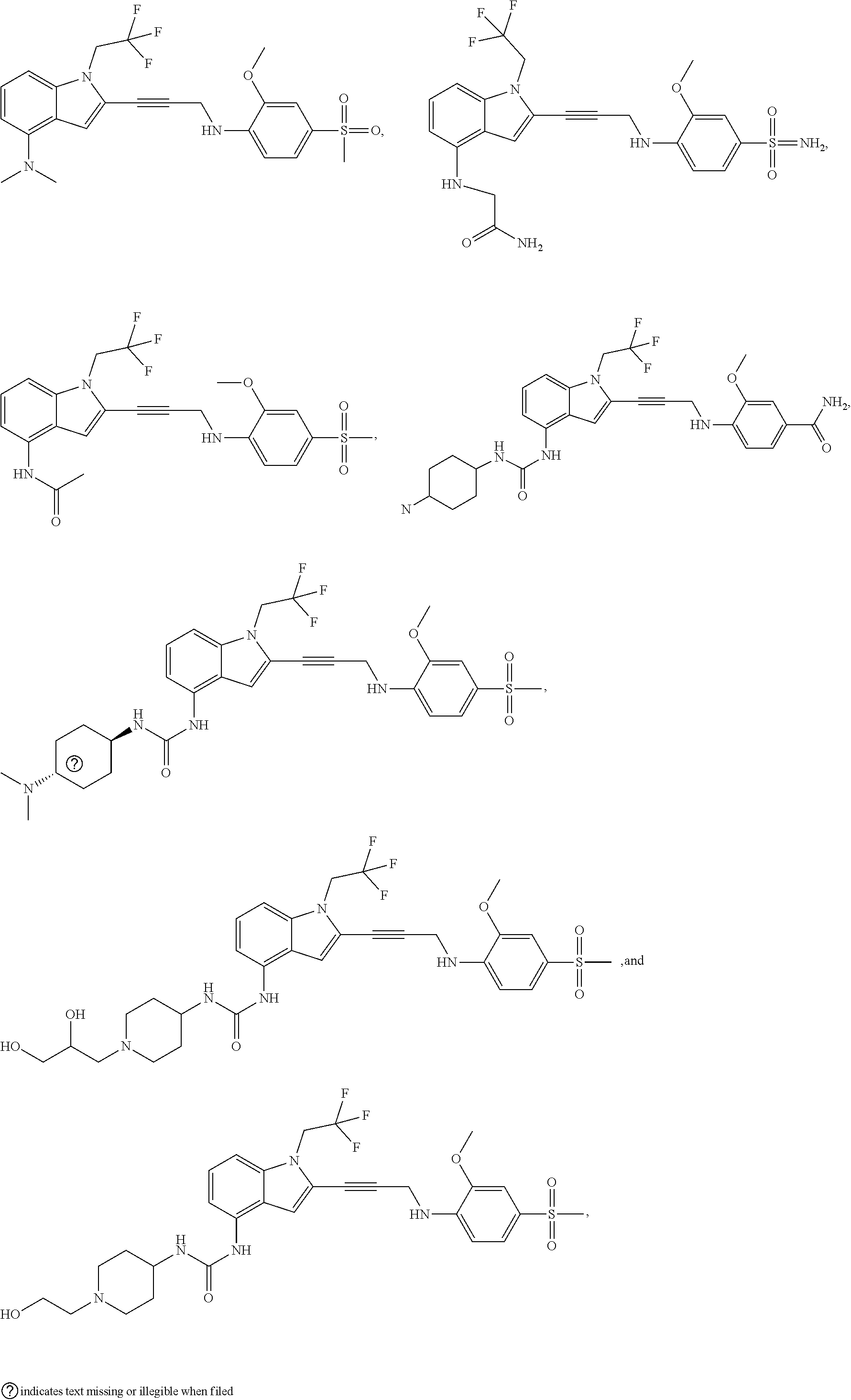

or a pharmaceutically-acceptable salt thereof.

Non-limiting examples of compounds of the current disclosure include the following:

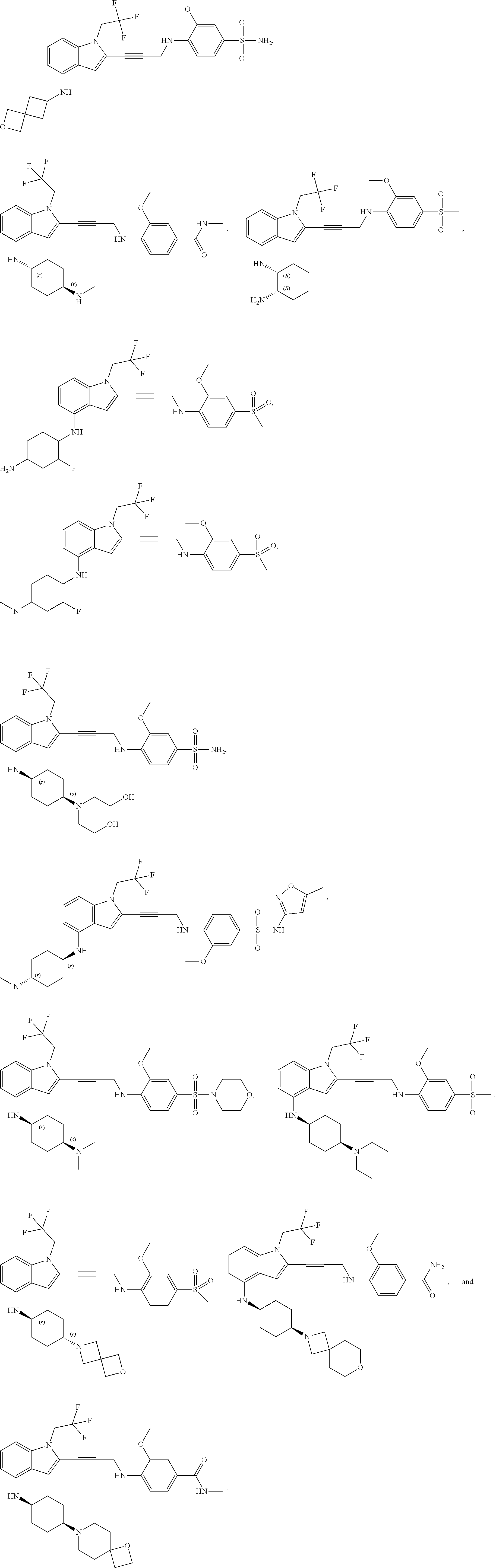

or a pharmaceutically-acceptable salt thereof.

Non-limiting examples of compounds of the current disclosure include the following:

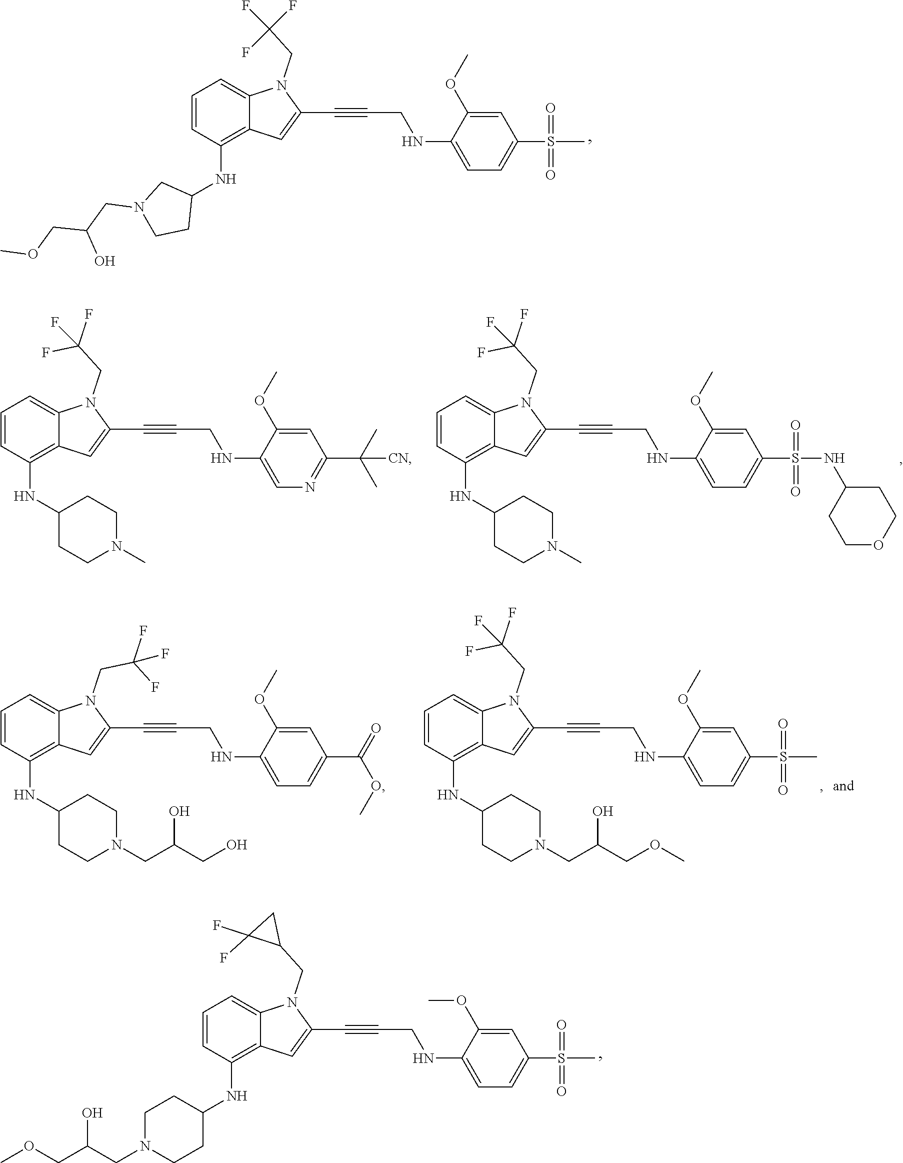

or a pharmaceutically-acceptable salt thereof.

Non-limiting examples of compounds of the current disclosure include the following:

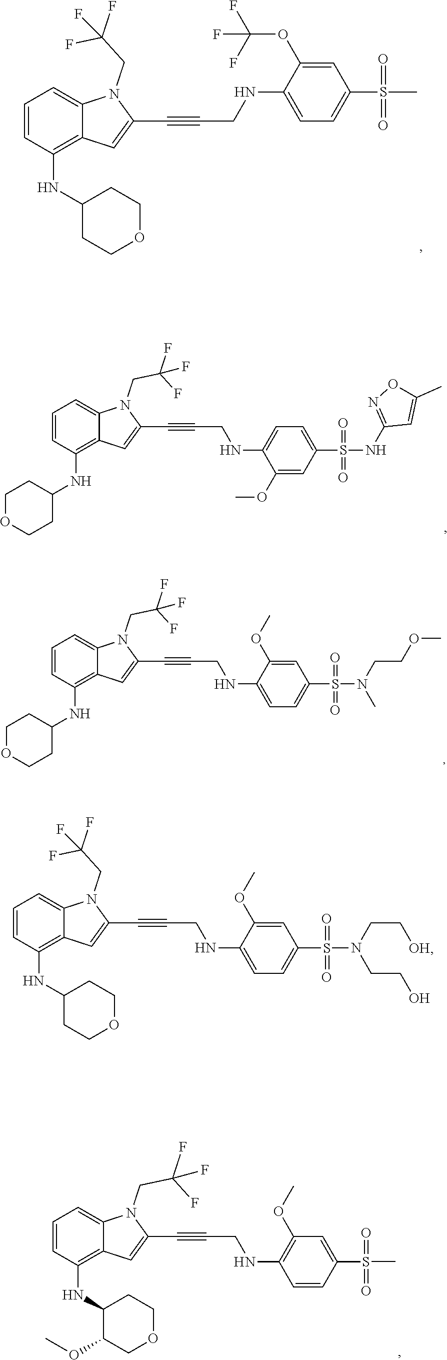

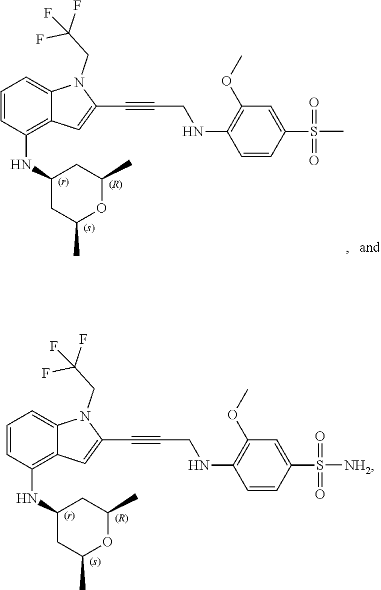

or a pharmaceutically-acceptable salt thereof.

Non-limiting examples of compounds of the current disclosure include the following:

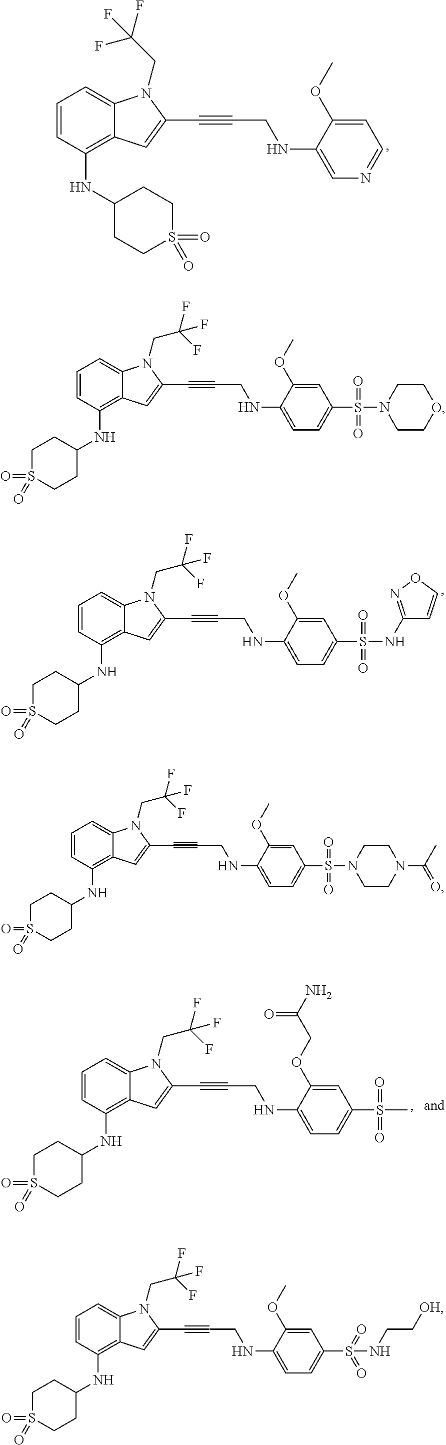 [SMILES: COc1ccncc1NCC#Cc1cc2c(NC3CCS(=O)(=O)CC3)cccc2n1CC(F)(F)F ; COc1cc(S(=O)(=O)N2CCOCC2)ccc1NCC#Cc1cc2c(NC3CCS(=O)(=O)CC3)cccc2n1CC(F)(F)F ; COc1cc(S(=O)(=O)Nc2ccon2)ccc1NCC#Cc1cc2c(NC3CCS(=O)(=O)CC3)cccc2n1CC(F)(F)F ; COc1cc(S(=O)(=O)N2CCN(C(C)=O)CC2)ccc1NCC#Cc1cc2c(NC3CCS(=O)(=O)CC3)cccc2n1CC(F)(F)F ; CS(=O)(=O)c1ccc(NCC#Cc2cc3c(NC4CCS(=O)(=O)CC4)cccc3n2CC(F)(F)F)c(OCC(N)=O)c1 ; COc1cc(S(=O)(=O)NCCO)ccc1NCC#Cc1cc2c(NC3CCS(=O)(=O)CC3)cccc2n1CC(F)(F)F]

or a pharmaceutically-acceptable salt thereof.

Non-limiting examples of compounds of the current disclosure include the following:

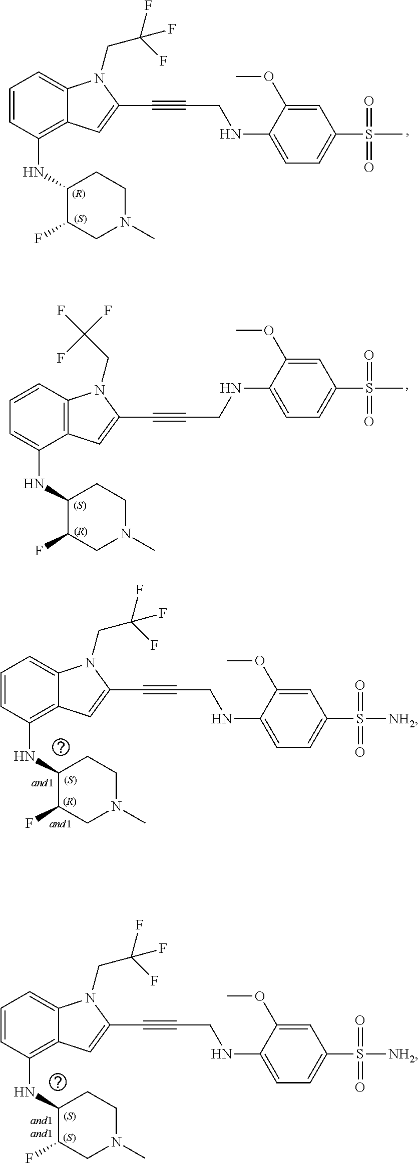

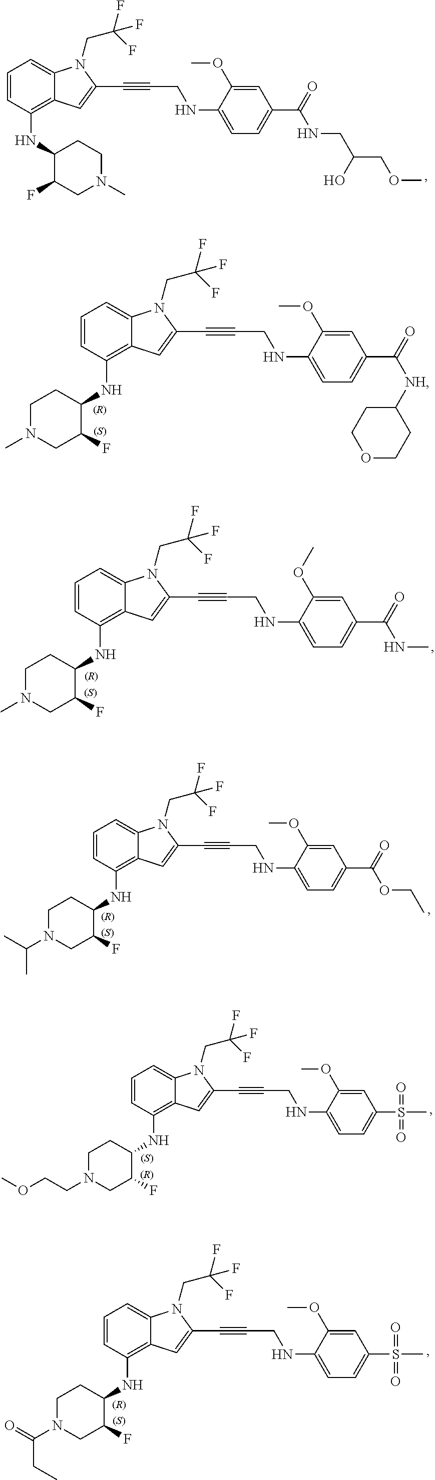

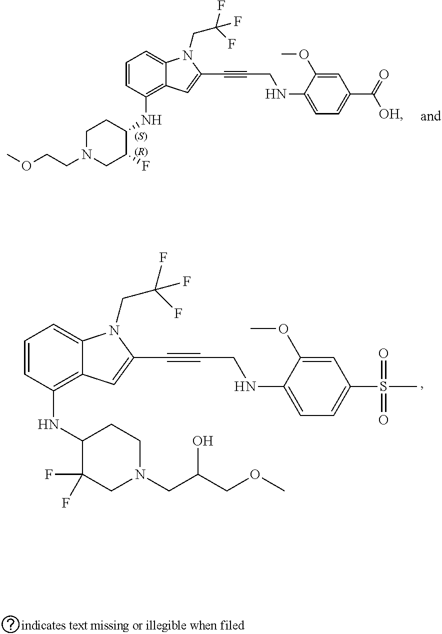

or a pharmaceutically-acceptable salt thereof.

Non-limiting examples of compounds of the current disclosure include the following:

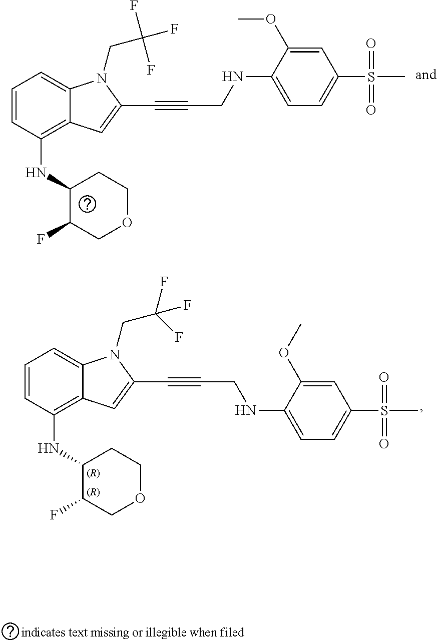

or a pharmaceutically-acceptable salt thereof.

Non-limiting examples of compounds of the current disclosure include the following:

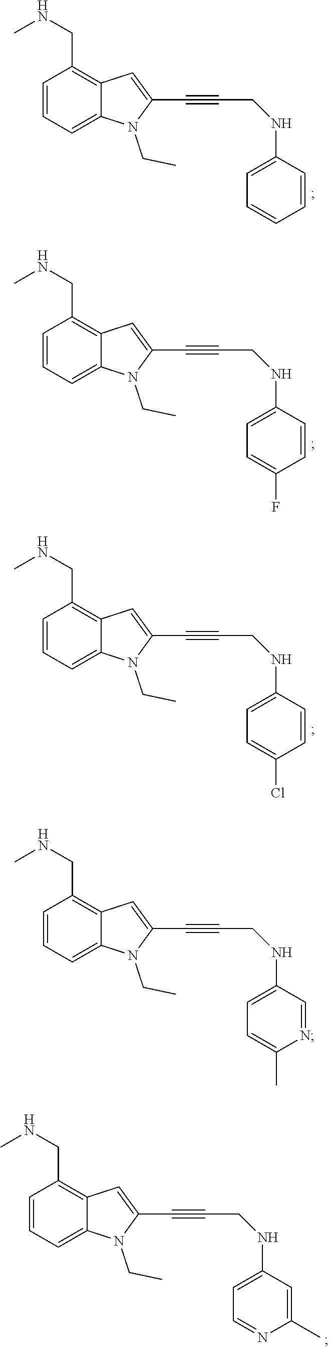

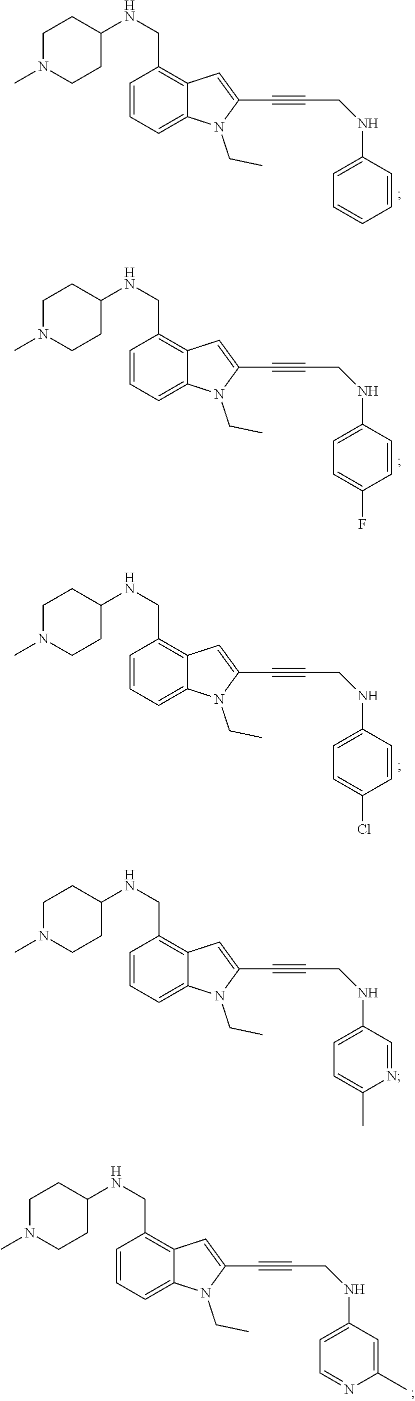

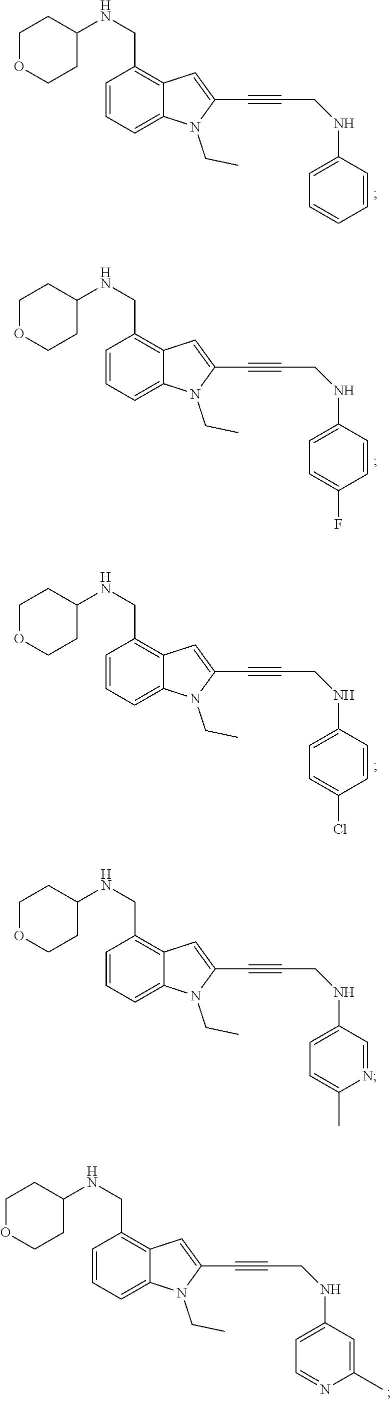 [SMILES: CCn1c(C#CCNc2ccccc2)cc2c(CNC3CCOCC3)cccc21 ; CCn1c(C#CCNc2ccc(F)cc2)cc2c(CNC3CCOCC3)cccc21 ; CCn1c(C#CCNc2ccc(Cl)cc2)cc2c(CNC3CCOCC3)cccc21 ; CCn1c(C#CCNc2ccc(C)nc2)cc2c(CNC3CCOCC3)cccc21 ; CCn1c(C#CCNc2ccnc(C)c2)cc2c(CNC3CCOCC3)cccc21]

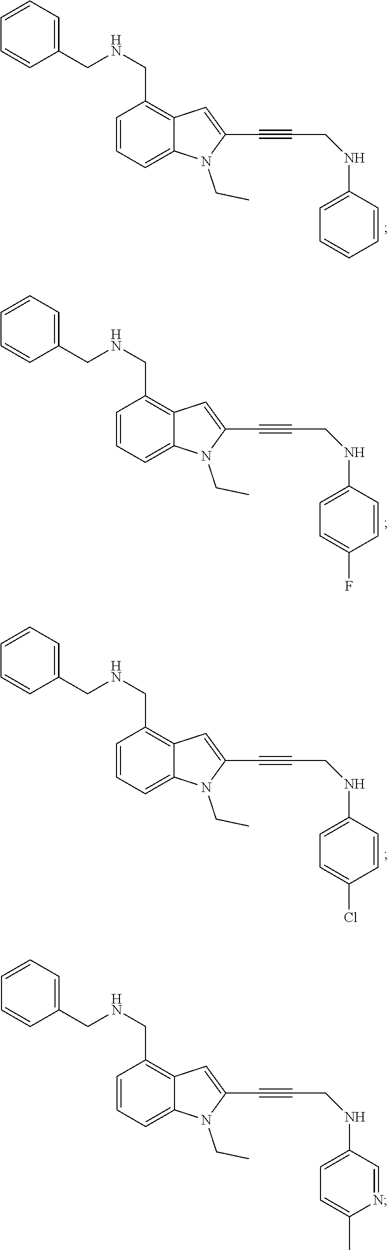 [SMILES: CCn1c(C#CCNc2ccccc2)cc2c(CNCc3ccccc3)cccc21 ; CCn1c(C#CCNc2ccc(F)cc2)cc2c(CNCc3ccccc3)cccc21 ; CCn1c(C#CCNc2ccc(Cl)cc2)cc2c(CNCc3ccccc3)cccc21 ; CCn1c(C#CCNc2ccc(C)nc2)cc2c(CNCc3ccccc3)cccc21]

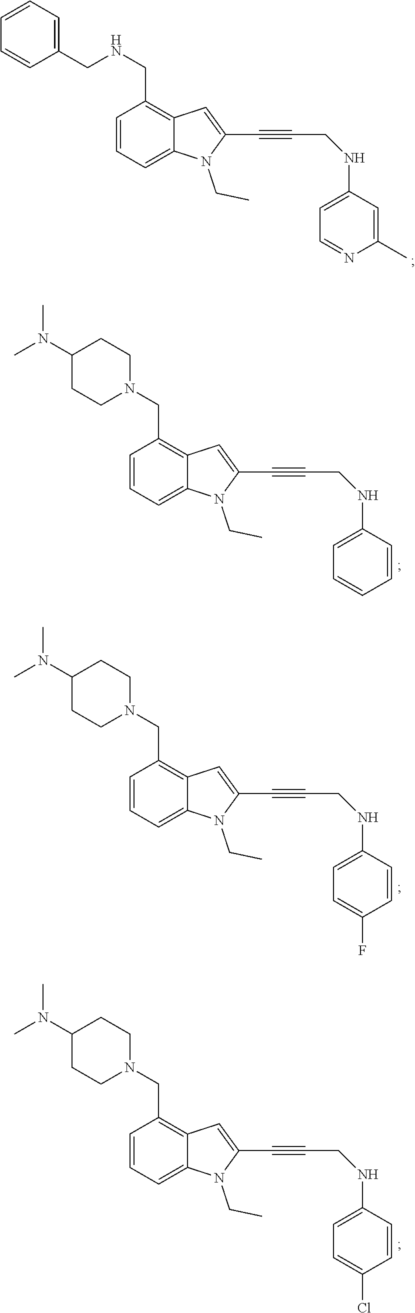

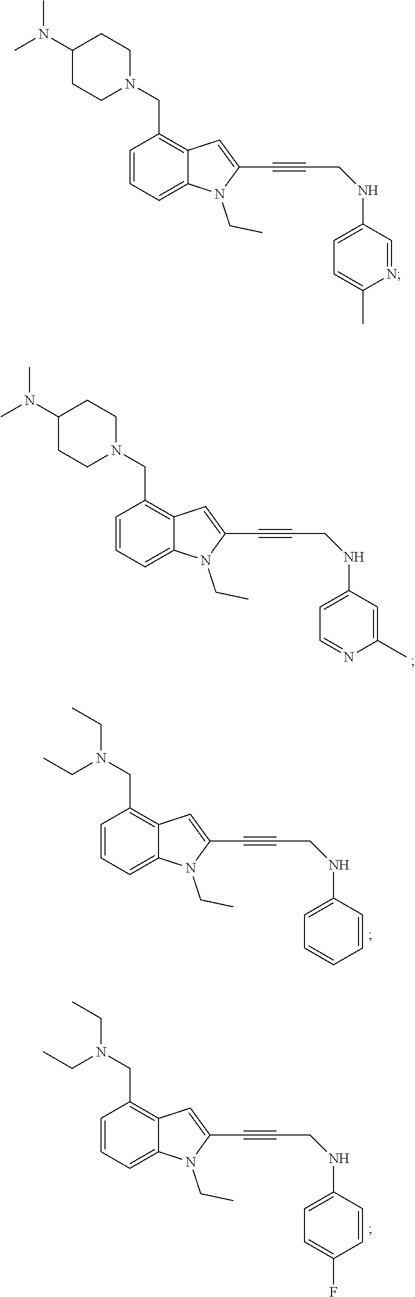

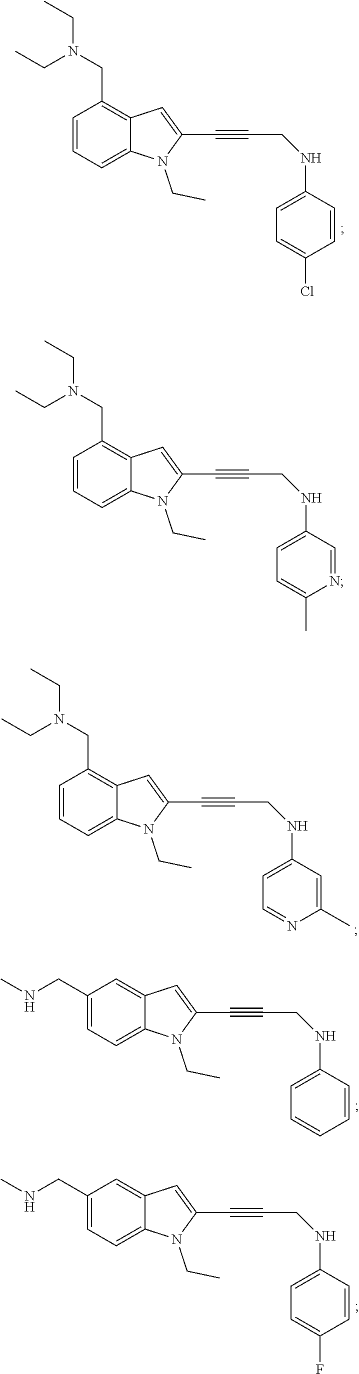

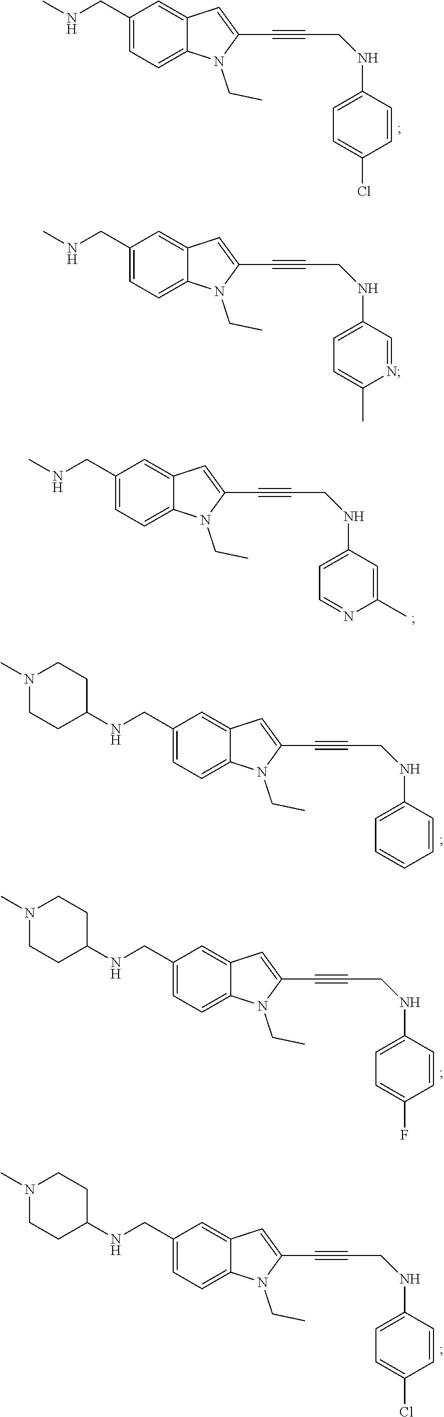

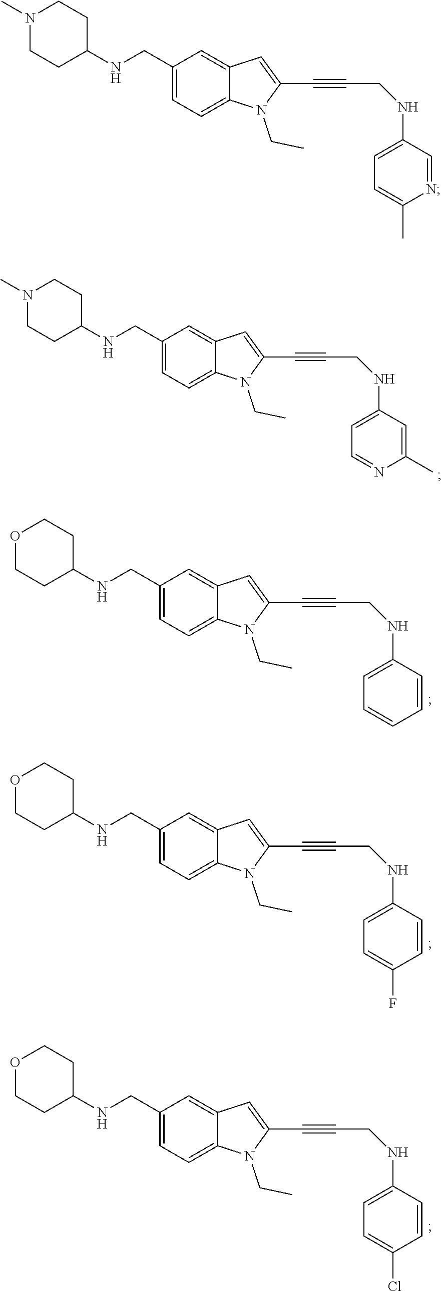

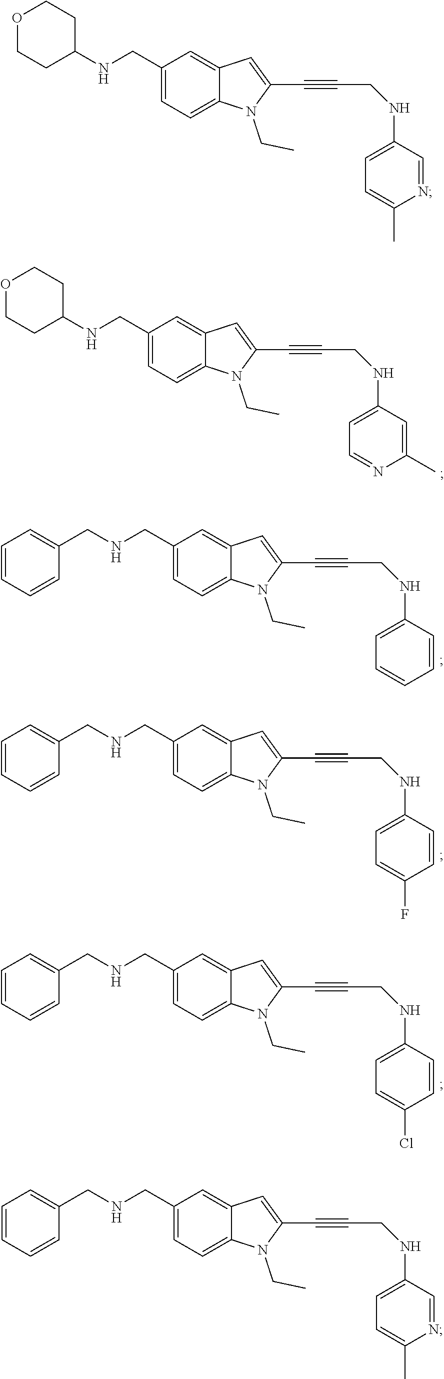 [SMILES: CCn1c(C#CCNc2ccc(C)nc2)cc2cc(CNC3CCOCC3)ccc21 ; CCn1c(C#CCNc2ccnc(C)c2)cc2cc(CNC3CCOCC3)ccc21 ; CCn1c(C#CCNc2ccccc2)cc2cc(CNCc3ccccc3)ccc21 ; CCn1c(C#CCNc2ccc(F)cc2)cc2cc(CNCc3ccccc3)ccc21 ; CCn1c(C#CCNc2ccc(Cl)cc2)cc2cc(CNCc3ccccc3)ccc21 ; CCn1c(C#CCNc2ccc(C)nc2)cc2cc(CNCc3ccccc3)ccc21]

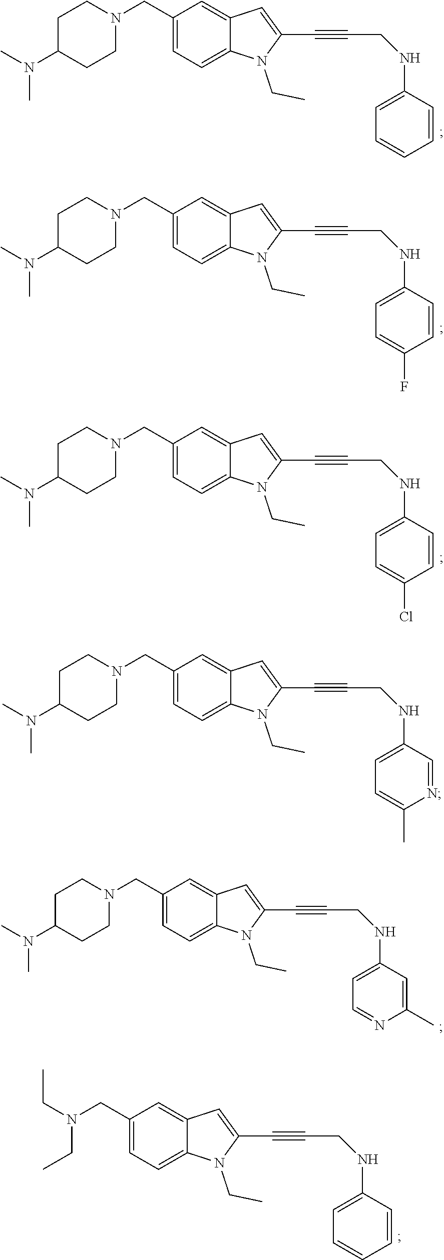

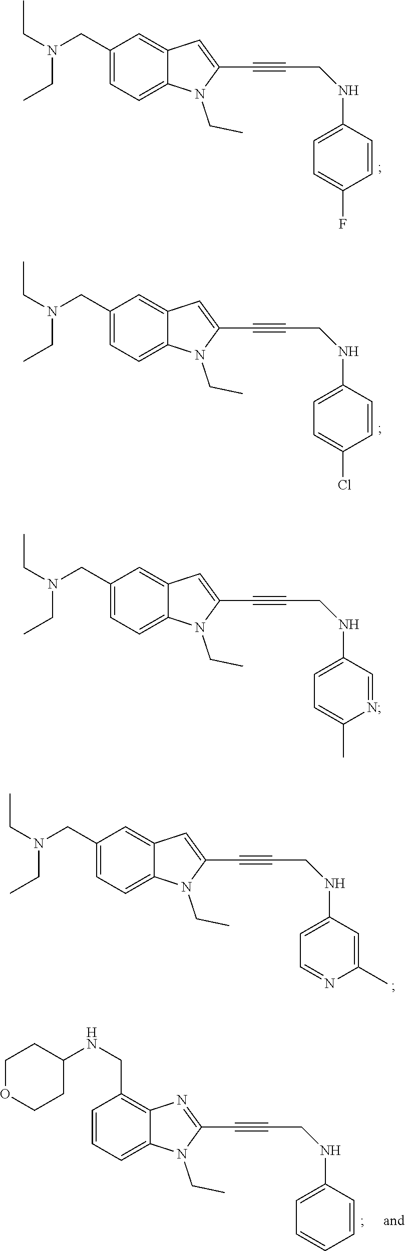 [SMILES: CCN(CC)Cc1ccc2c(c1)cc(C#CCNc1ccc(F)cc1)n2CC ; CCN(CC)Cc1ccc2c(c1)cc(C#CCNc1ccc(Cl)cc1)n2CC ; CCN(CC)Cc1ccc2c(c1)cc(C#CCNc1ccc(C)nc1)n2CC ; CCN(CC)Cc1ccc2c(c1)cc(C#CCNc1ccnc(C)c1)n2CC ; CCn1c(C#CCNc2ccccc2)nc2c(CNC3CCOCC3)cccc21]

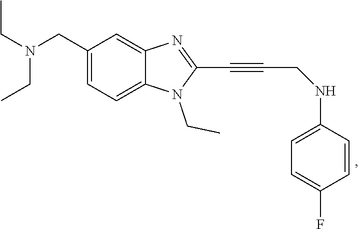 [SMILES: CCN(CC)Cc1ccc2c(c1)nc(C#CCNc1ccc(F)cc1)n2CC]

or a pharmaceutically-acceptable salt of any of the foregoing.

Non-limiting examples of compounds of the current disclosure include the following:

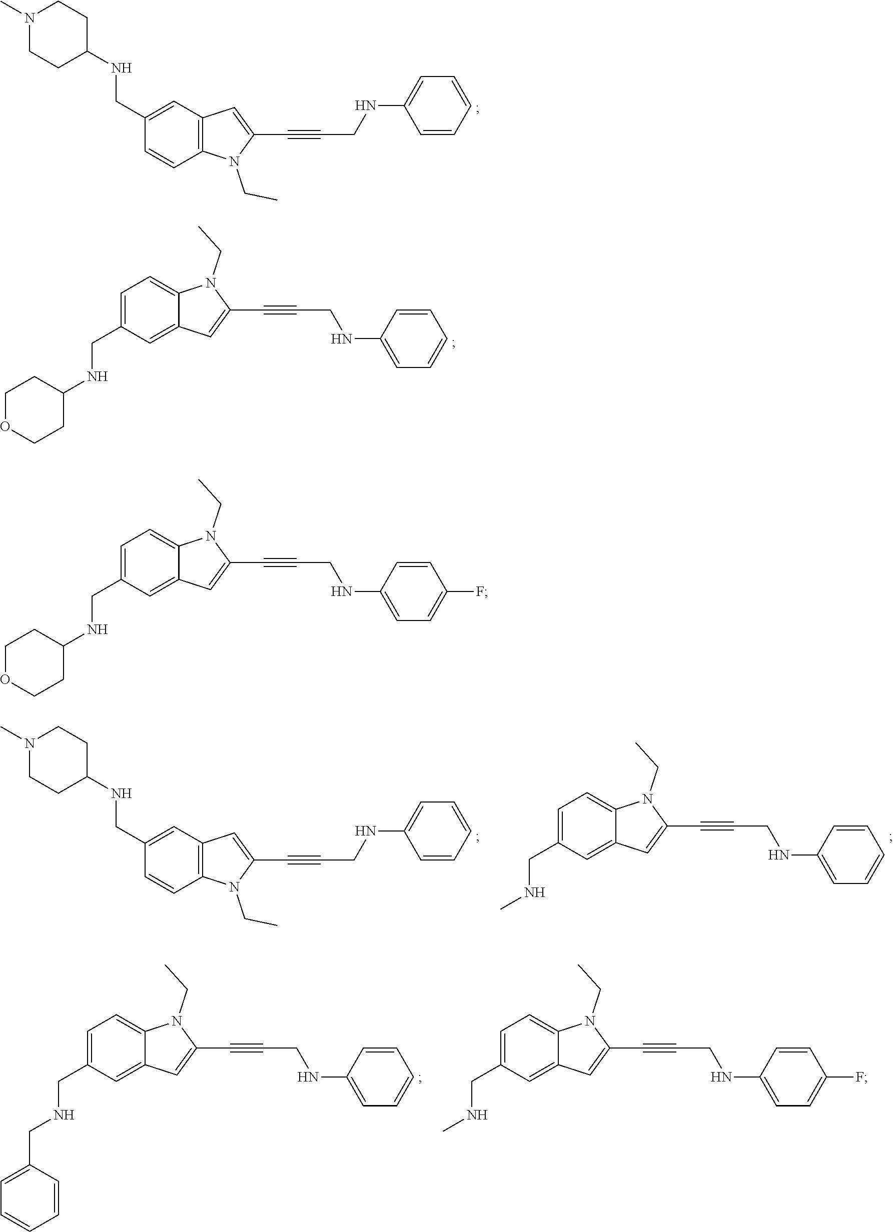

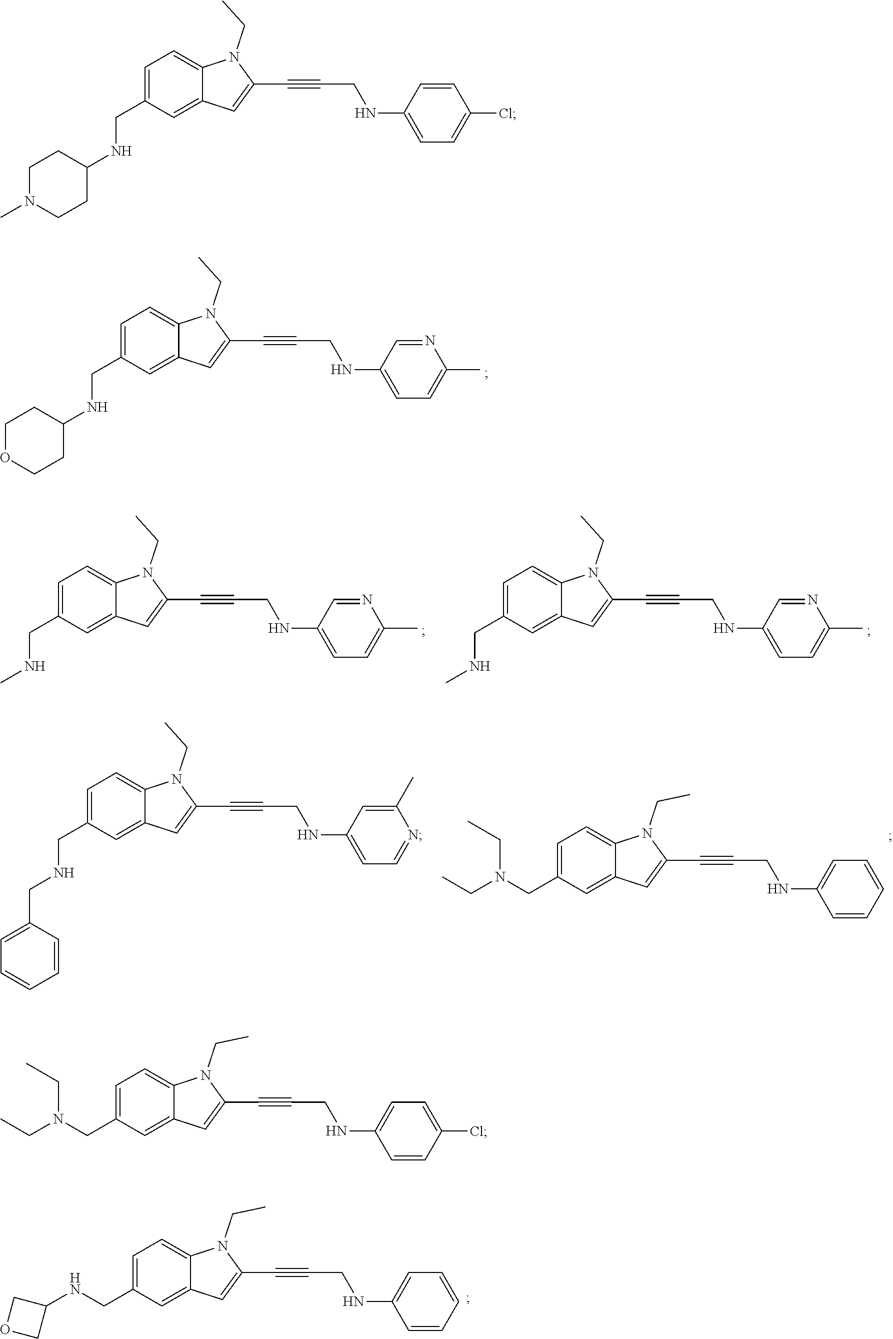

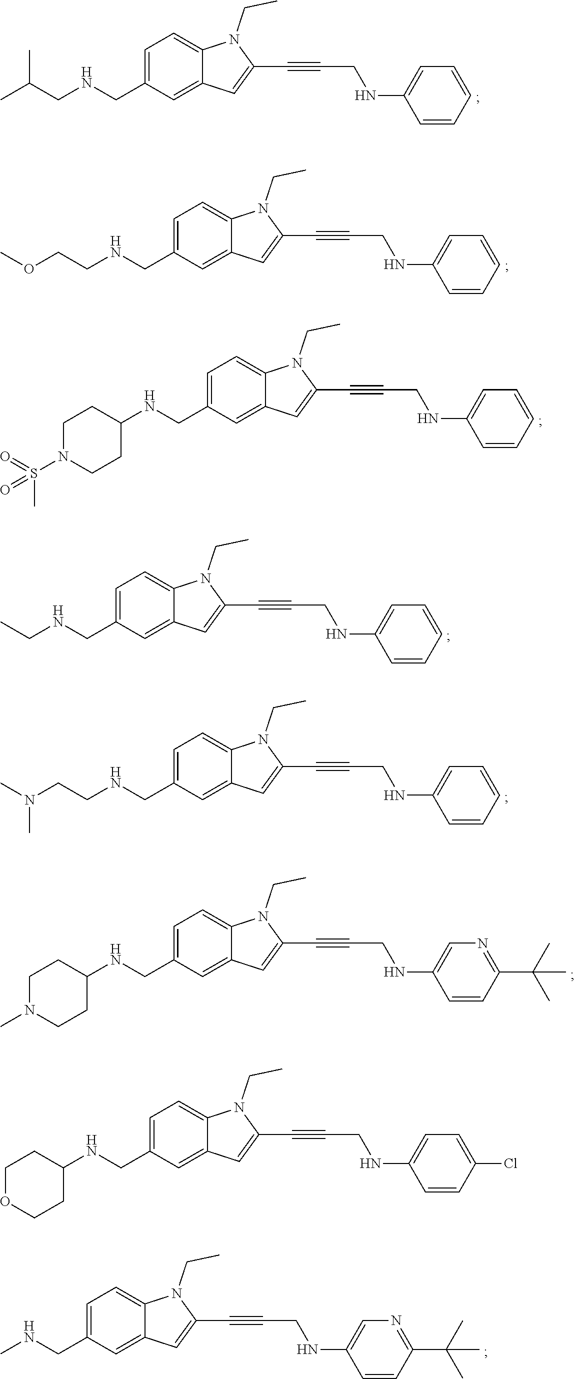

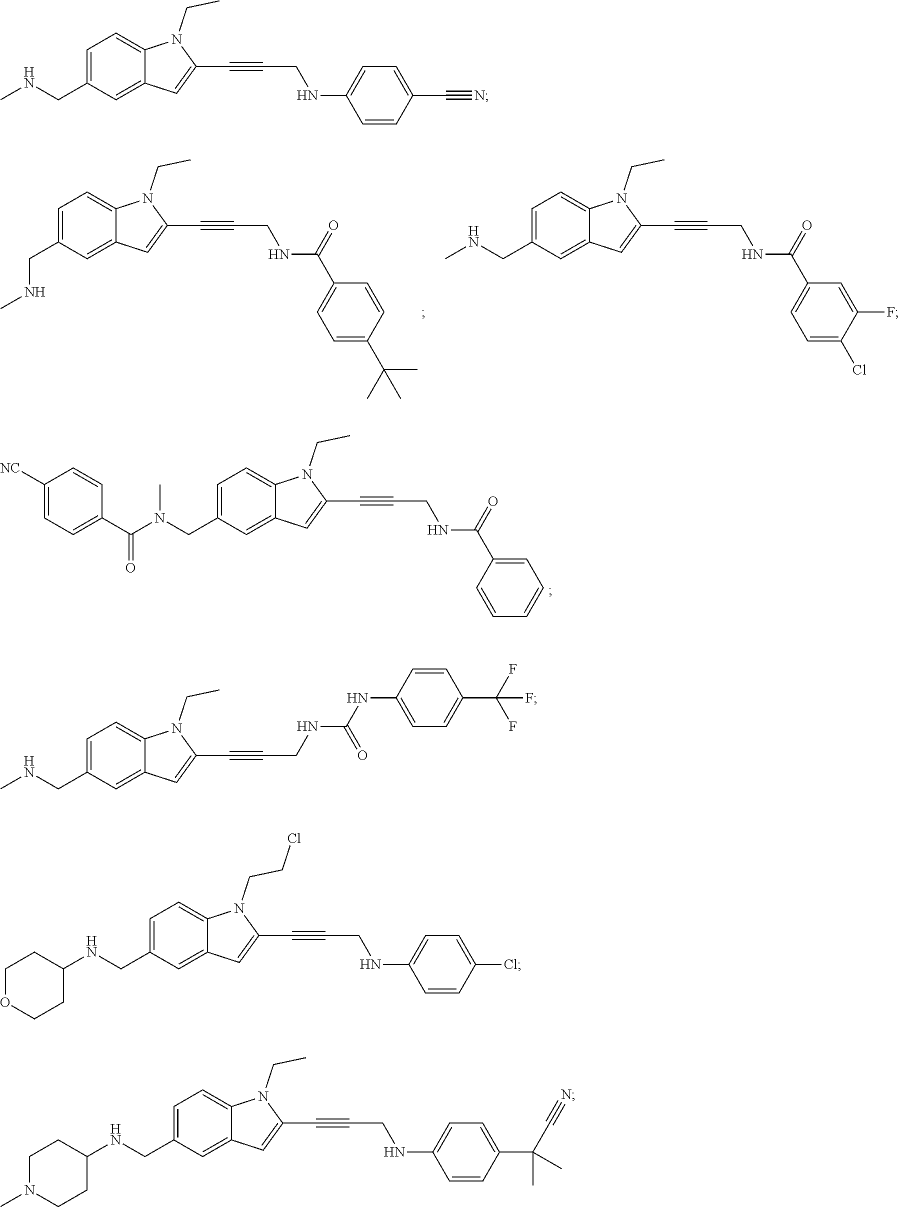

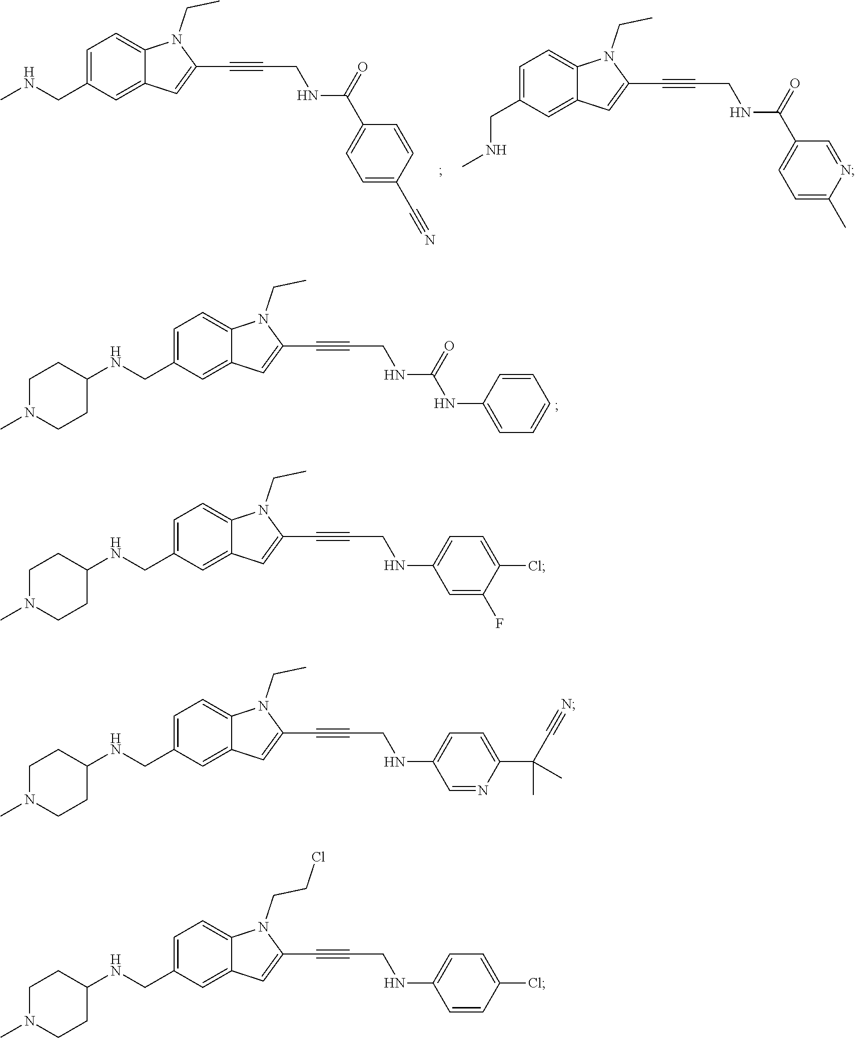

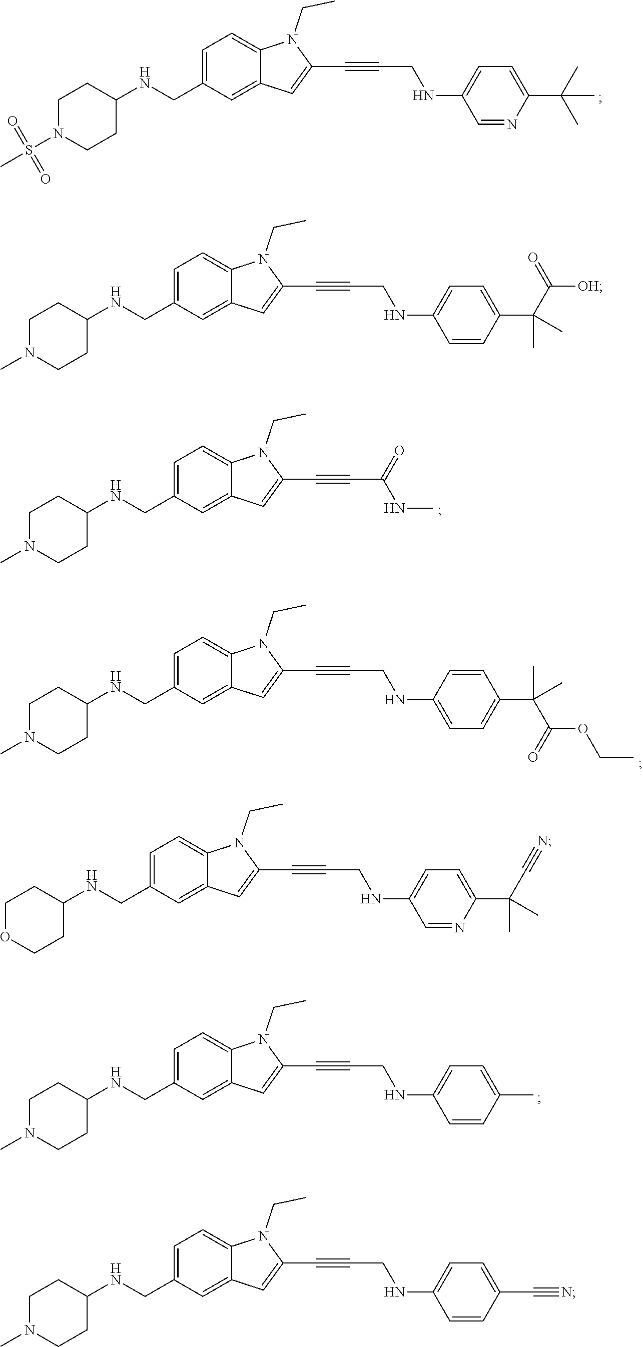

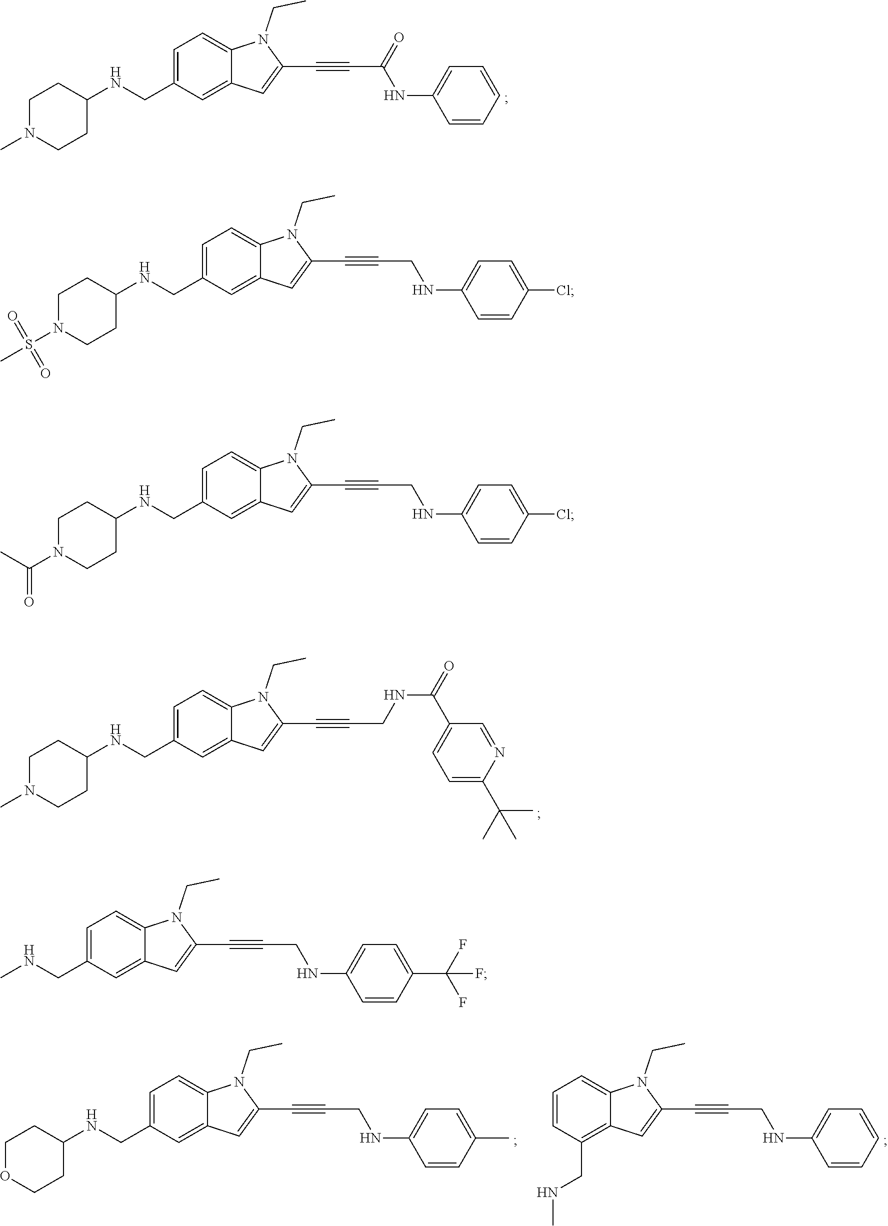

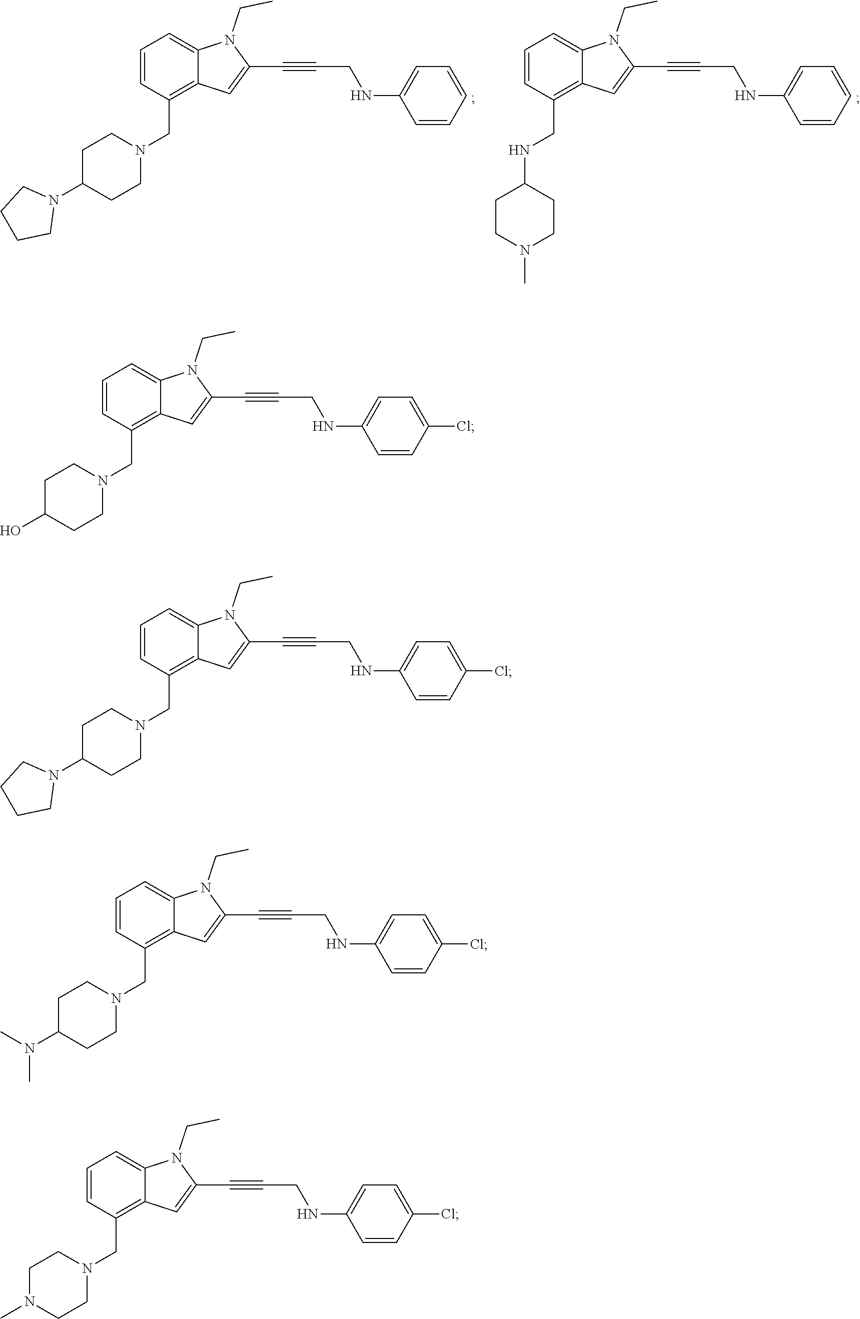 [SMILES: CCn1c(C#CCNc2ccccc2)cc2c(CN3CCC(N4CCCC4)CC3)cccc21 ; CCn1c(C#CCNc2ccccc2)cc2c(CNC3CCN(C)CC3)cccc21 ; CCn1c(C#CCNc2ccc(Cl)cc2)cc2c(CN3CCC(O)CC3)cccc21 ; CCn1c(C#CCNc2ccc(Cl)cc2)cc2c(CN3CCC(N4CCCC4)CC3)cccc21 ; CCn1c(C#CCNc2ccc(Cl)cc2)cc2c(CN3CCC(N(C)C)CC3)cccc21 ; CCn1c(C#CCNc2ccc(Cl)cc2)cc2c(CN3CCN(C)CC3)cccc21]

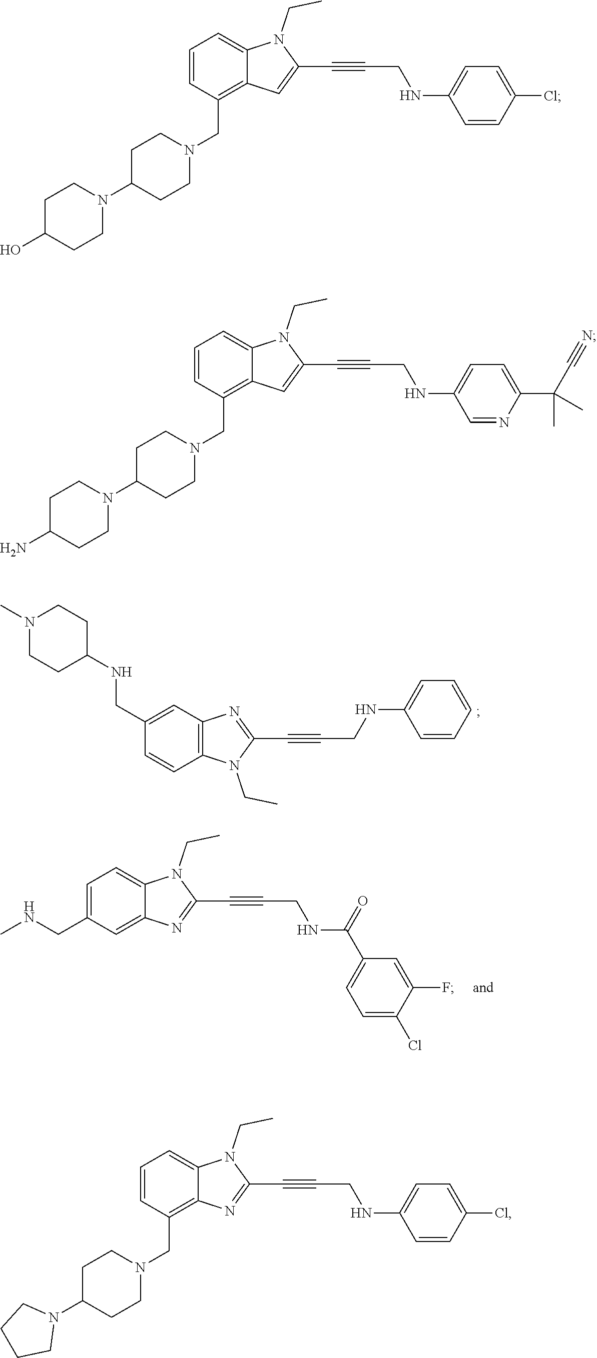

or a pharmaceutically-acceptable salt of any of the foregoing.

Non-limiting examples of compounds of the current disclosure include the following:

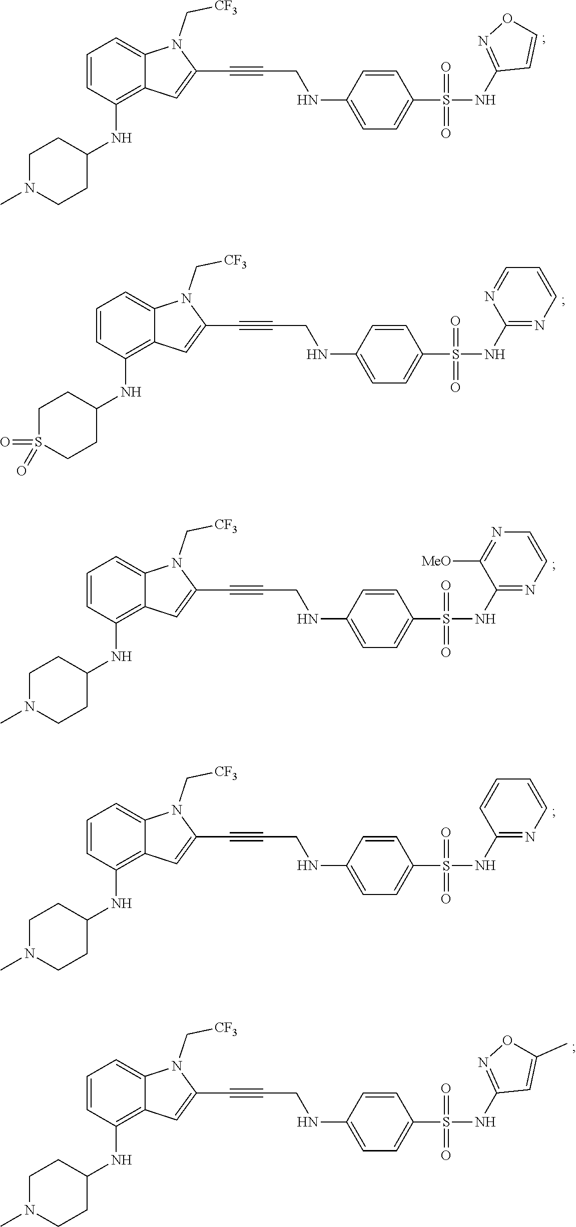

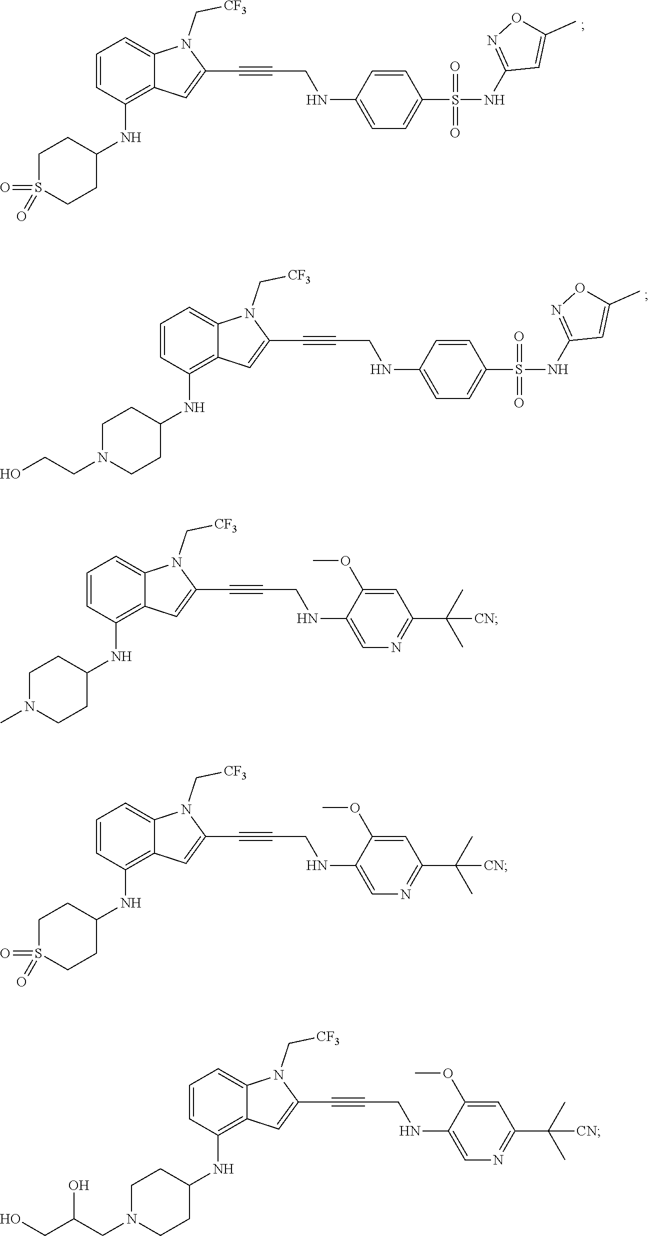

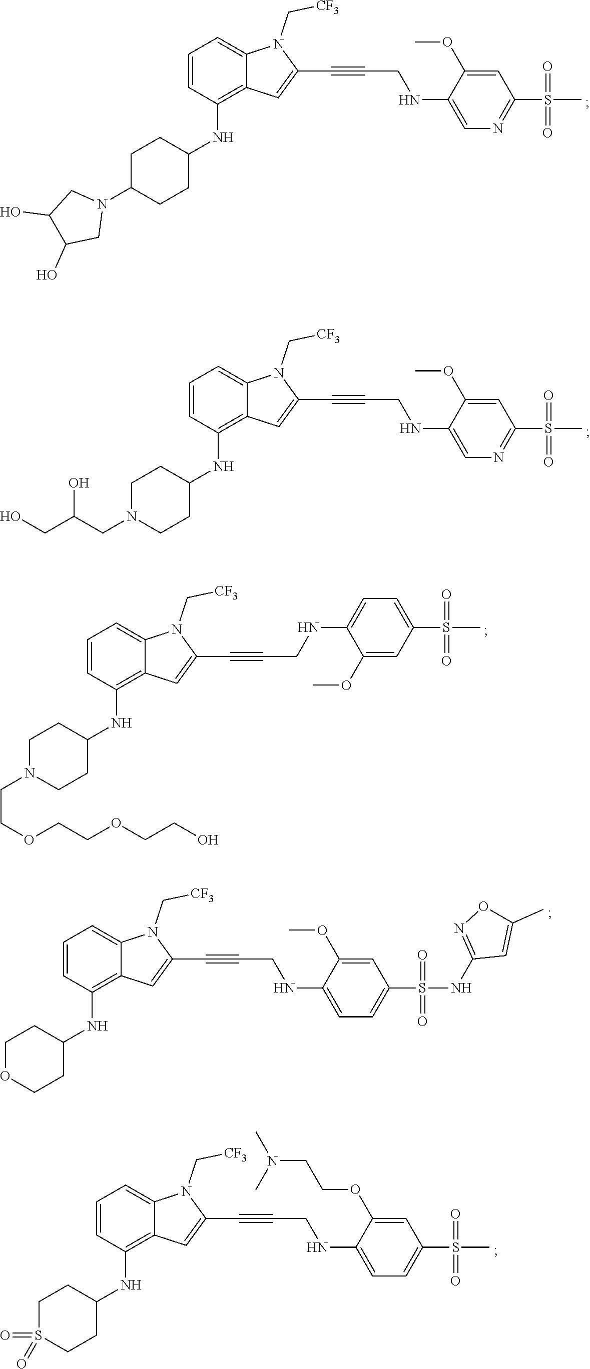 [SMILES: COc1cc(S(C)(=O)=O)ncc1NCC#Cc1cc2c(NC3CCC(N4CC(O)C(O)C4)CC3)cccc2n1CC(F)(F)F ; COc1cc(S(C)(=O)=O)ncc1NCC#Cc1cc2c(NC3CCN(CC(O)CO)CC3)cccc2n1CC(F)(F)F ; COc1cc(S(C)(=O)=O)ccc1NCC#Cc1cc2c(NC3CCN(CCOCCOCCO)CC3)cccc2n1CC(F)(F)F ; COc1cc(S(=O)(=O)Nc2cc(C)on2)ccc1NCC#Cc1cc2c(NC3CCOCC3)cccc2n1CC(F)(F)F ; CN(C)CCOc1cc(S(C)(=O)=O)ccc1NCC#Cc1cc2c(NC3CCS(=O)(=O)CC3)cccc2n1CC(F)(F)F]

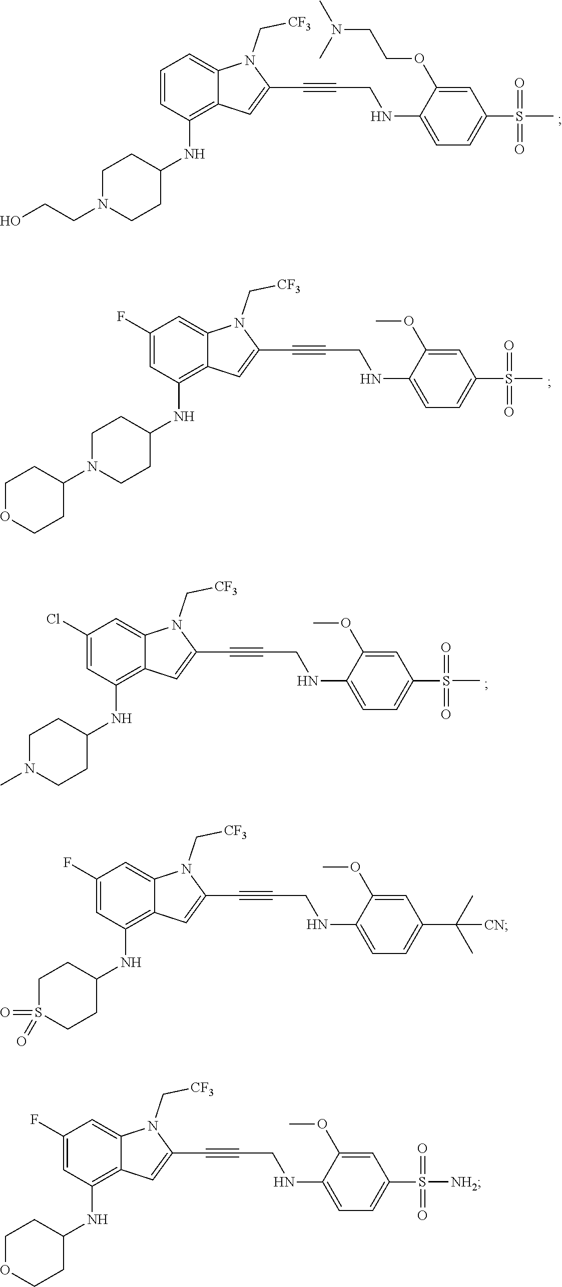

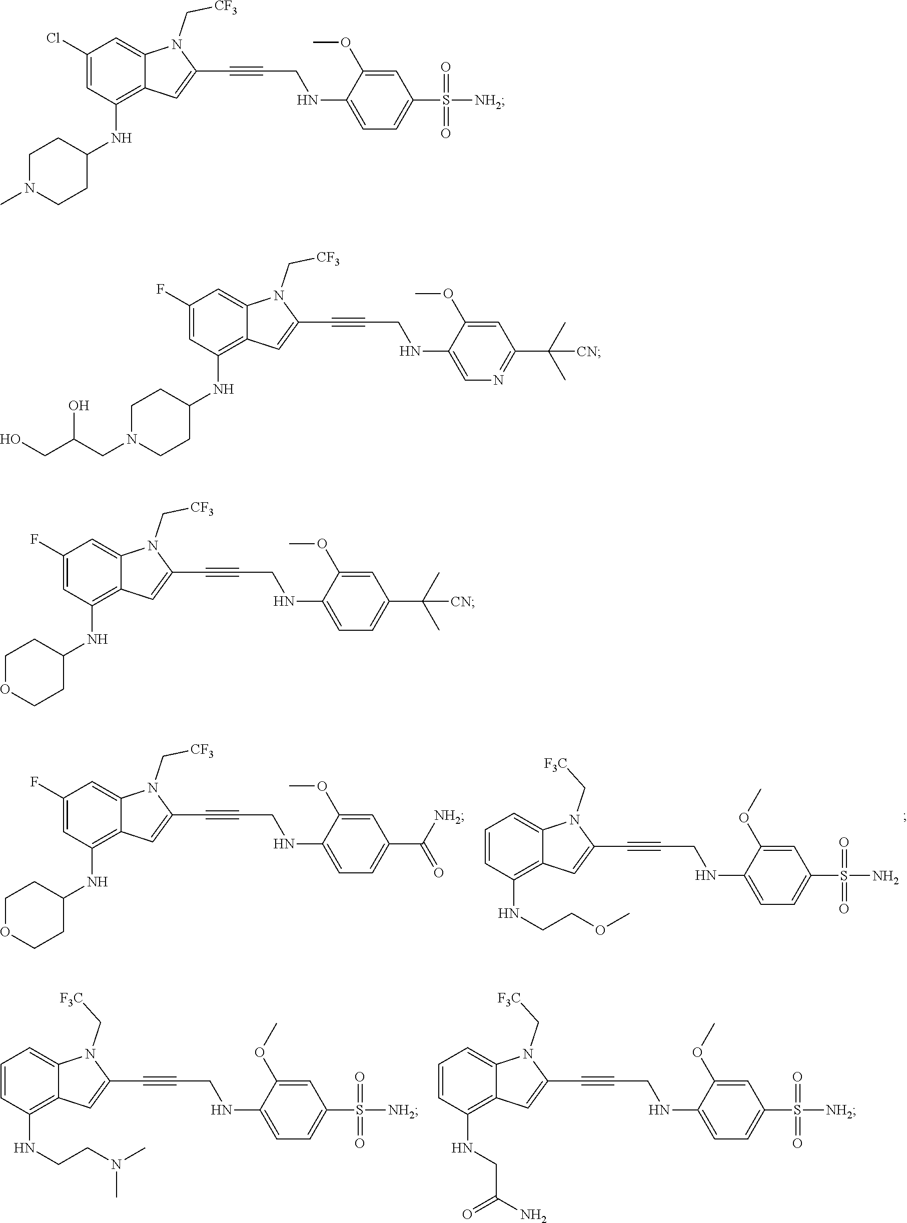

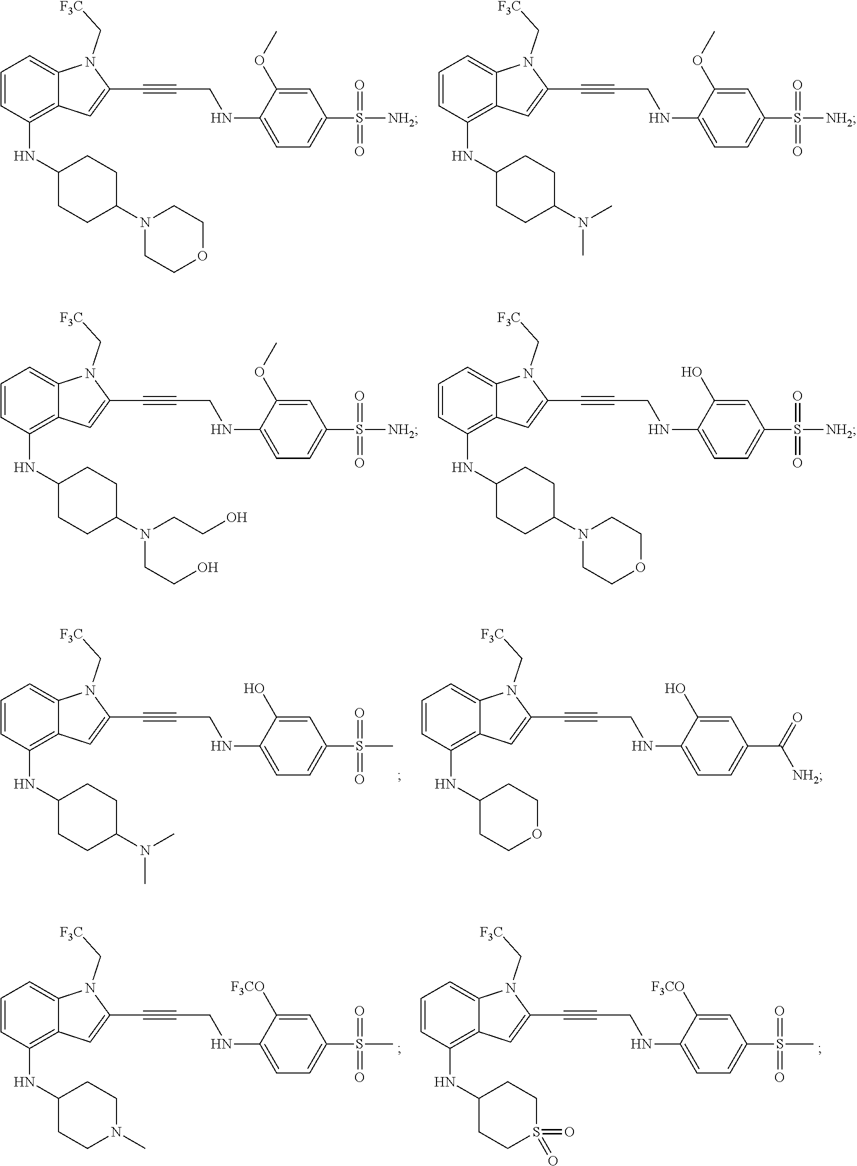

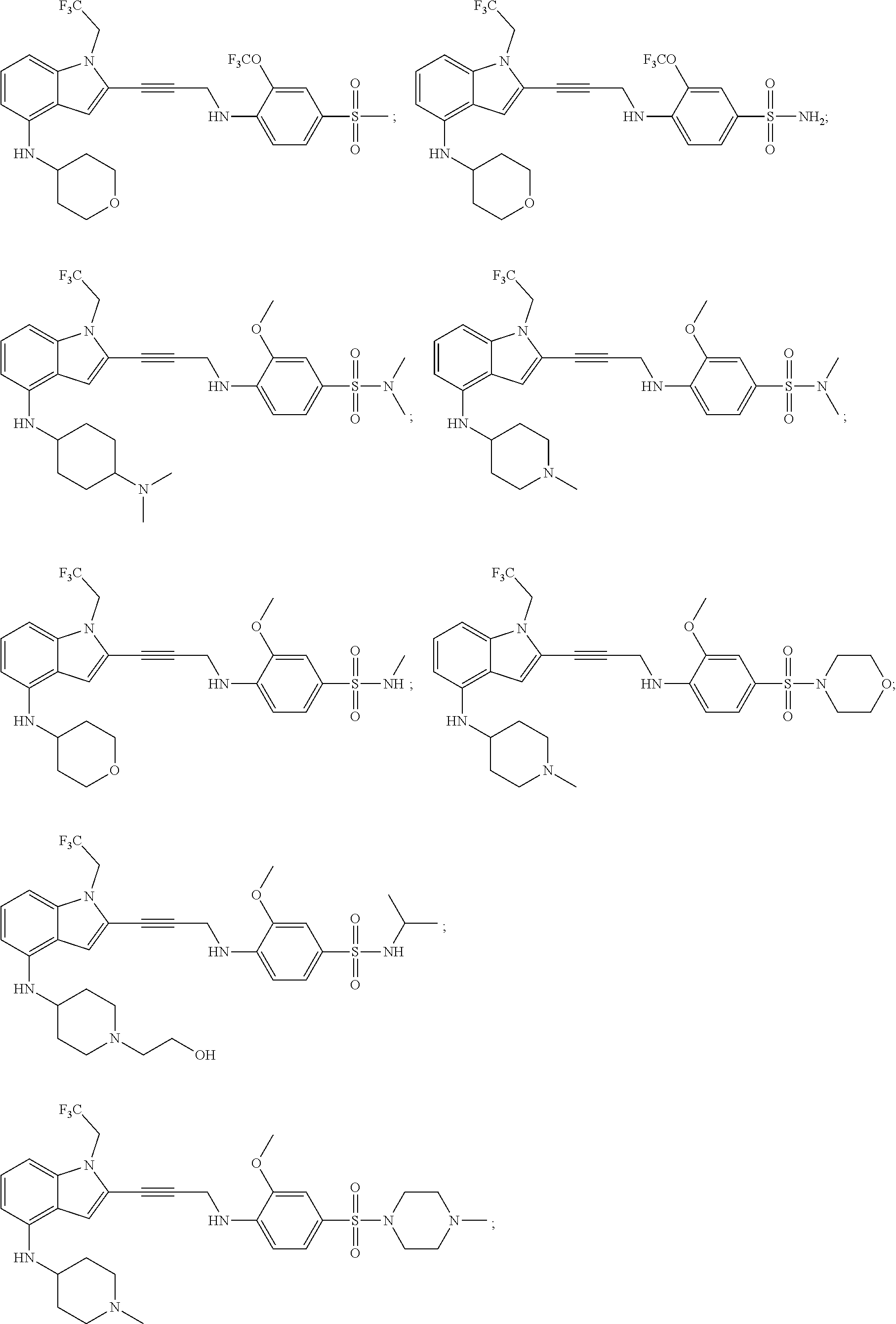

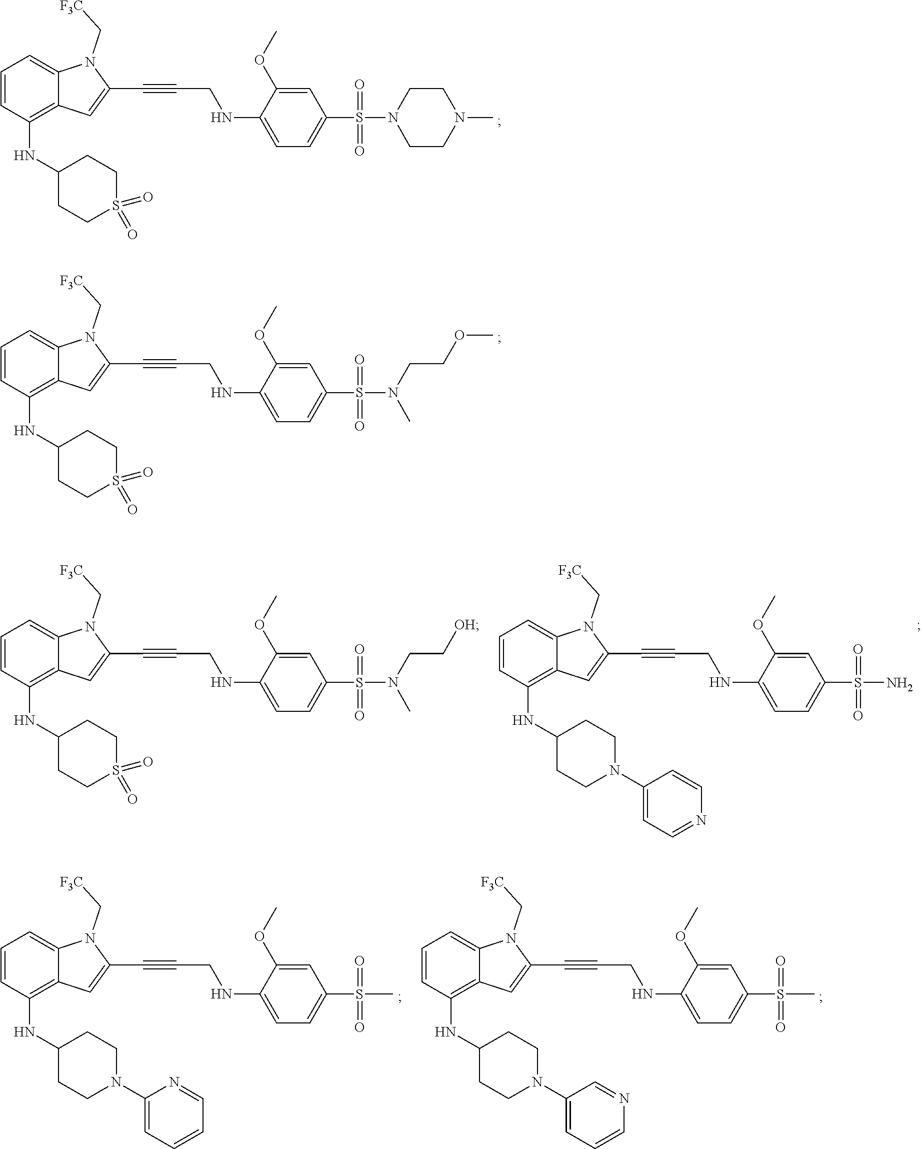

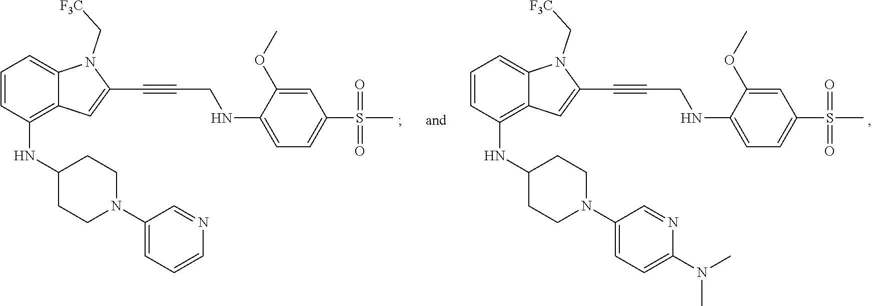 [SMILES: COc1cc(S(C)(=O)=O)ccc1NCC#Cc1cc2c(NC3CCN(c4cccnc4)CC3)cccc2n1CC(F)(F)F ; COc1cc(S(C)(=O)=O)ccc1NCC#Cc1cc2c(NC3CCN(c4ccc(N(C)C)nc4)CC3)cccc2n1CC(F)(F)F]

or a pharmaceutically-acceptable salt of any of the forgoing.

Non-limiting examples of compounds of the current disclosure include the following:

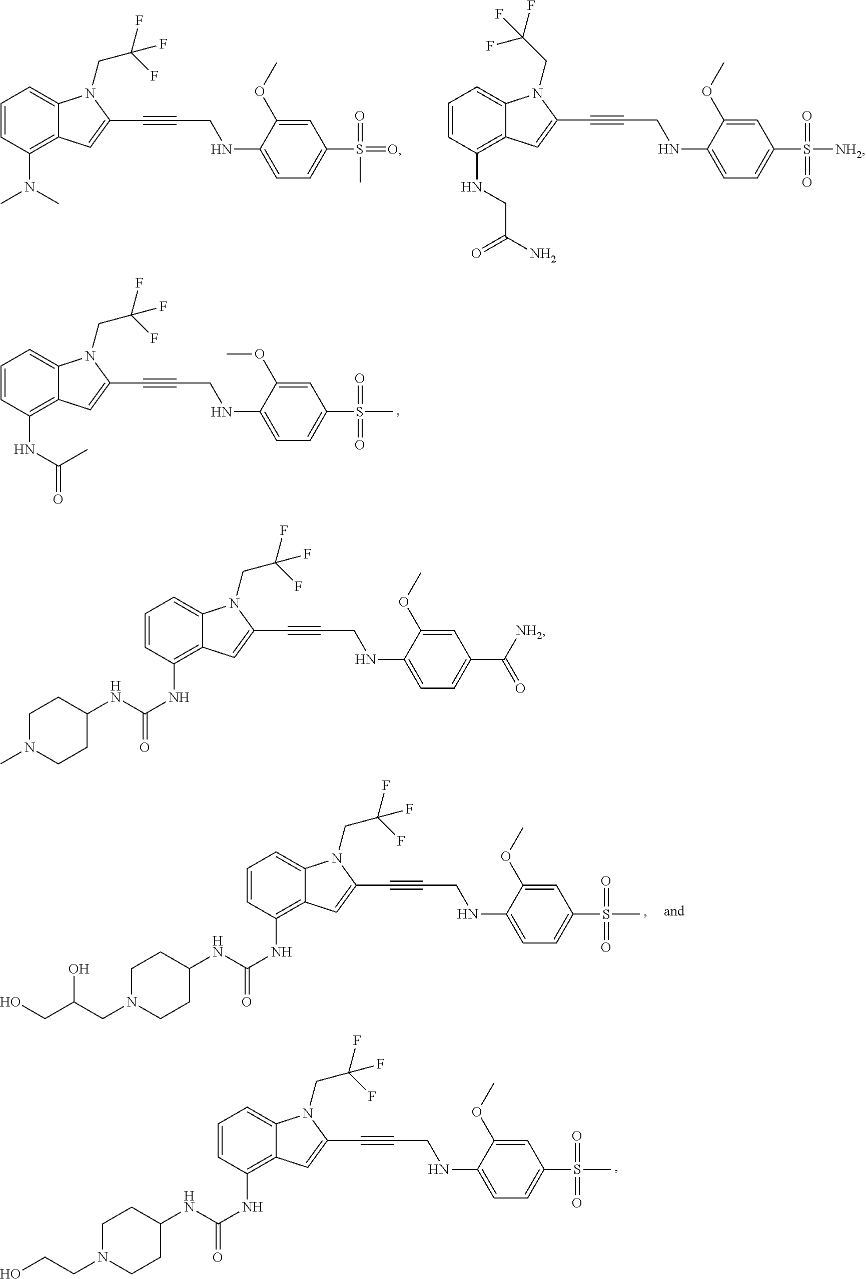 [SMILES: COc1cc(S(C)(=O)=O)ccc1NCC#Cc1cc2c(N(C)C)cccc2n1CC(F)(F)F ; COc1cc(S(N)(=O)=O)ccc1NCC#Cc1cc2c(NCC(N)=O)cccc2n1CC(F)(F)F ; COc1cc(S(C)(=O)=O)ccc1NCC#Cc1cc2c(NC(C)=O)cccc2n1CC(F)(F)F ; COc1cc(C(N)=O)ccc1NCC#Cc1cc2c(NC(=O)NC3CCN(C)CC3)cccc2n1CC(F)(F)F ; COc1cc(S(C)(=O)=O)ccc1NCC#Cc1cc2c(NC(=O)NC3CCN(CC(O)CO)CC3)cccc2n1CC(F)(F)F ; COc1cc(S(C)(=O)=O)ccc1NCC#Cc1cc2c(NC(=O)NC3CCN(CCO)CC3)cccc2n1CC(F)(F)F]

or a pharmaceutically-acceptable salt thereof.

Non-limiting examples of compounds of the current disclosure include the following:

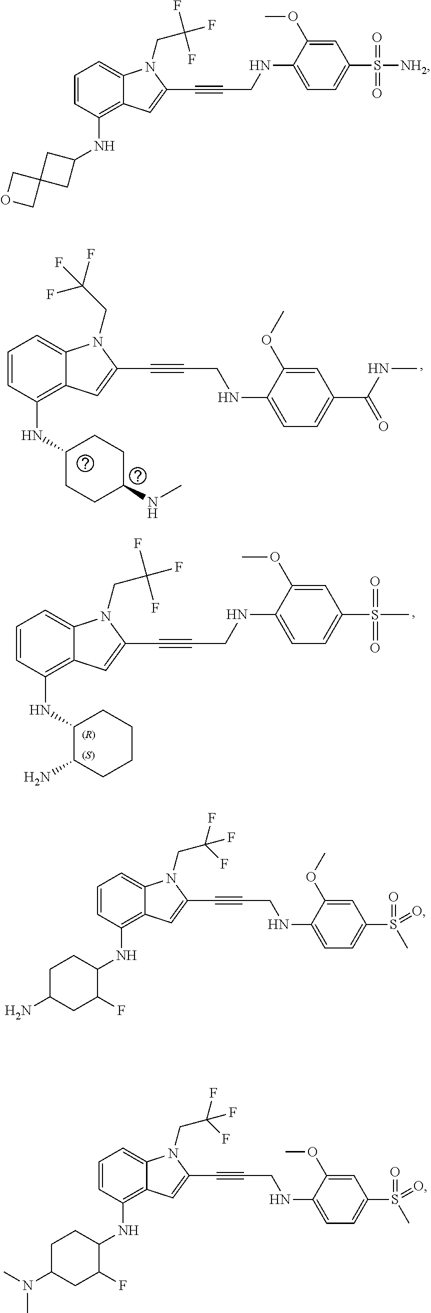

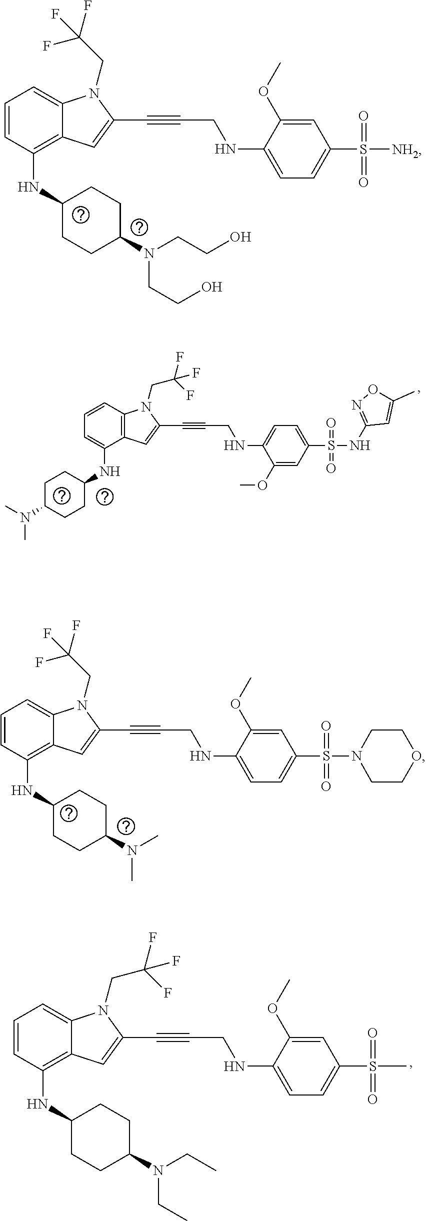

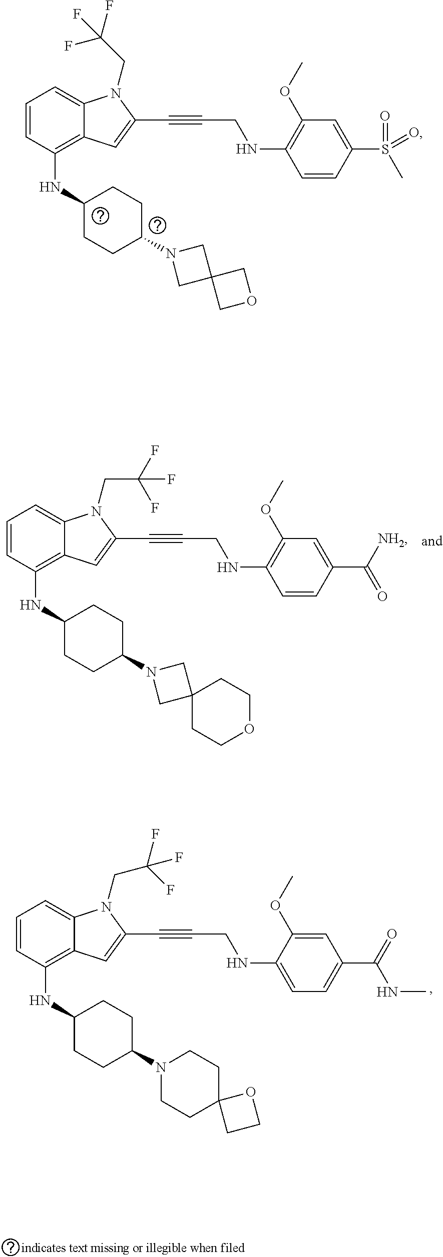 [SMILES: COc1cc(S(C)(=O)=O)ccc1NCC#Cc1cc2c(N[C@H]3CC[C@H](N4CC5(COC5)C4)CC3)cccc2n1CC(F)(F)F ; COc1cc(C(N)=O)ccc1NCC#Cc1cc2c(N[C@H]3CC[C@@H](N4CC5(CCOCC5)C4)CC3)cccc2n1CC(F)(F)F ; CNC(=O)c1ccc(NCC#Cc2cc3c(N[C@H]4CC[C@@H](N5CCC6(CCO6)CC5)CC4)cccc3n2CC(F)(F)F)c(OC)c1]

or a pharmaceutically-acceptable salt thereof.

Non-limiting examples of compounds of the current disclosure include the following:

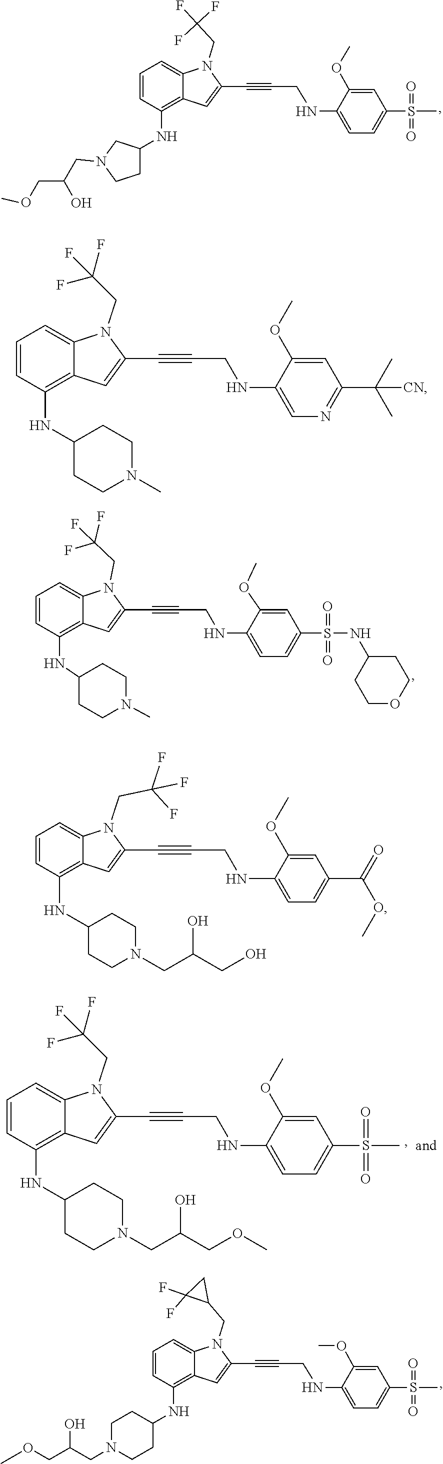

or a pharmaceutically-acceptable salt thereof.

Non-limiting examples of compounds of the current disclosure include the following:

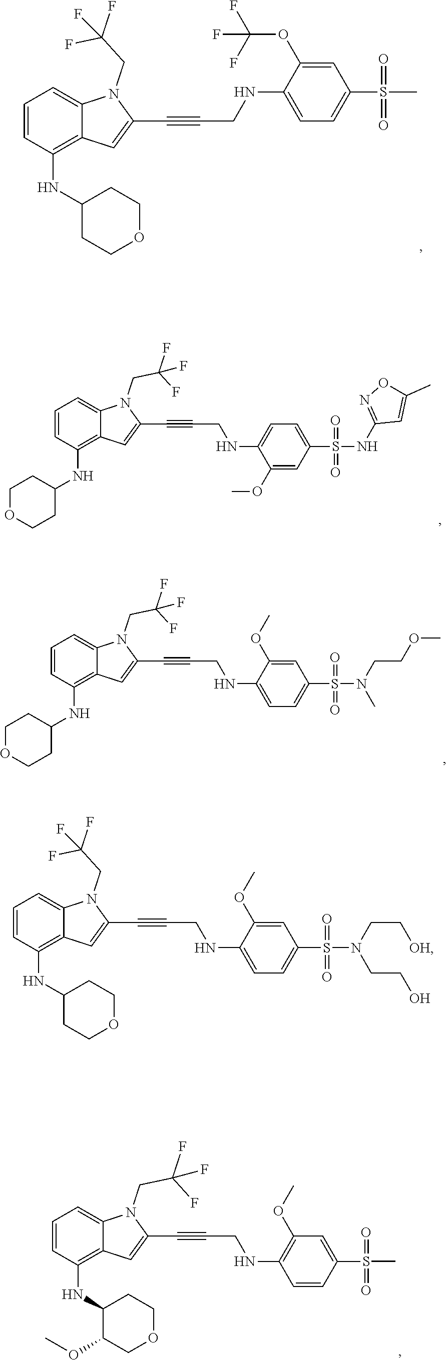

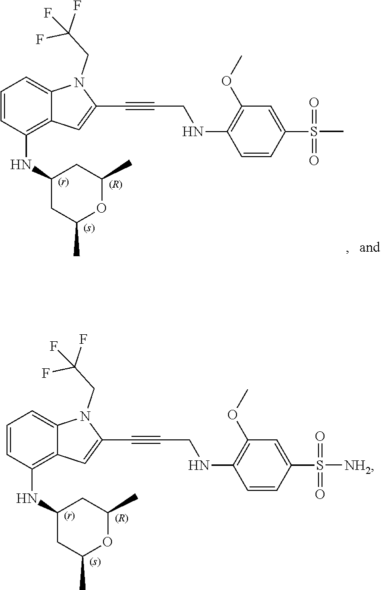

or a pharmaceutically-acceptable salt thereof.

Non-limiting examples of compounds of the current disclosure include the following:

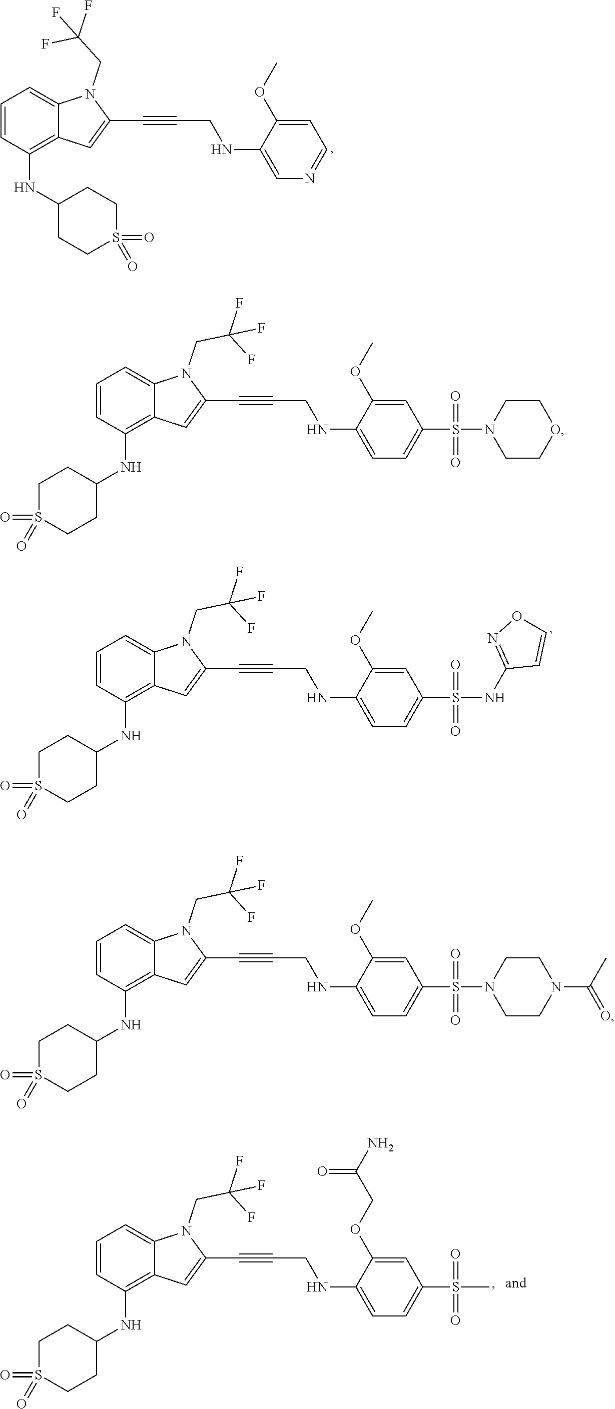 [SMILES: COc1ccncc1NCC#Cc1cc2c(NC3CCS(=O)(=O)CC3)cccc2n1CC(F)(F)F ; COc1cc(S(=O)(=O)N2CCOCC2)ccc1NCC#Cc1cc2c(NC3CCS(=O)(=O)CC3)cccc2n1CC(F)(F)F ; COc1cc(S(=O)(=O)Nc2ccon2)ccc1NCC#Cc1cc2c(NC3CCS(=O)(=O)CC3)cccc2n1CC(F)(F)F ; COc1cc(S(=O)(=O)N2CCN(C(C)=O)CC2)ccc1NCC#Cc1cc2c(NC3CCS(=O)(=O)CC3)cccc2n1CC(F)(F)F ; CS(=O)(=O)c1ccc(NCC#Cc2cc3c(NC4CCS(=O)(=O)CC4)cccc3n2CC(F)(F)F)c(OCC(N)=O)c1]

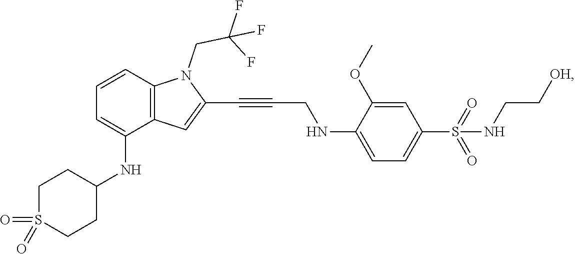 [SMILES: COc1cc(S(=O)(=O)NCCO)ccc1NCC#Cc1cc2c(NC3CCS(=O)(=O)CC3)cccc2n1CC(F)(F)F]

or a pharmaceutically-acceptable salt thereof.

Non-limiting examples of compounds of the current disclosure include the following:

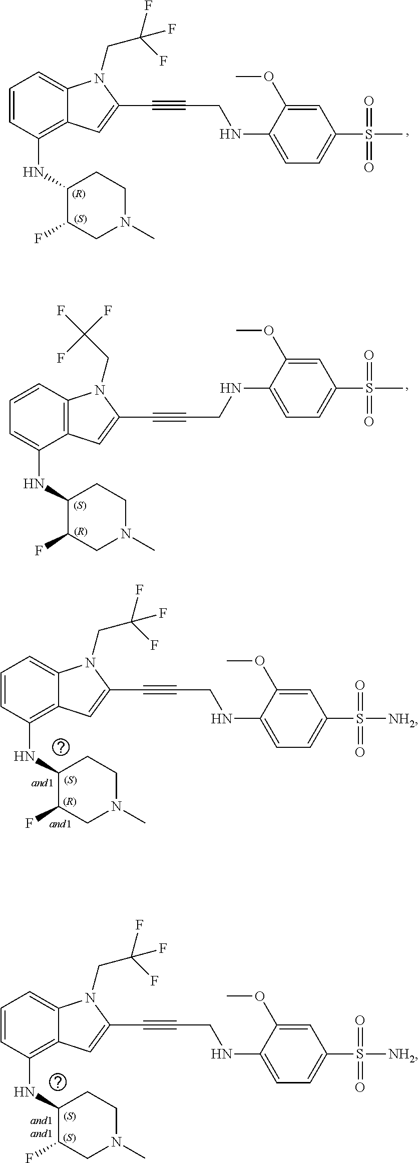

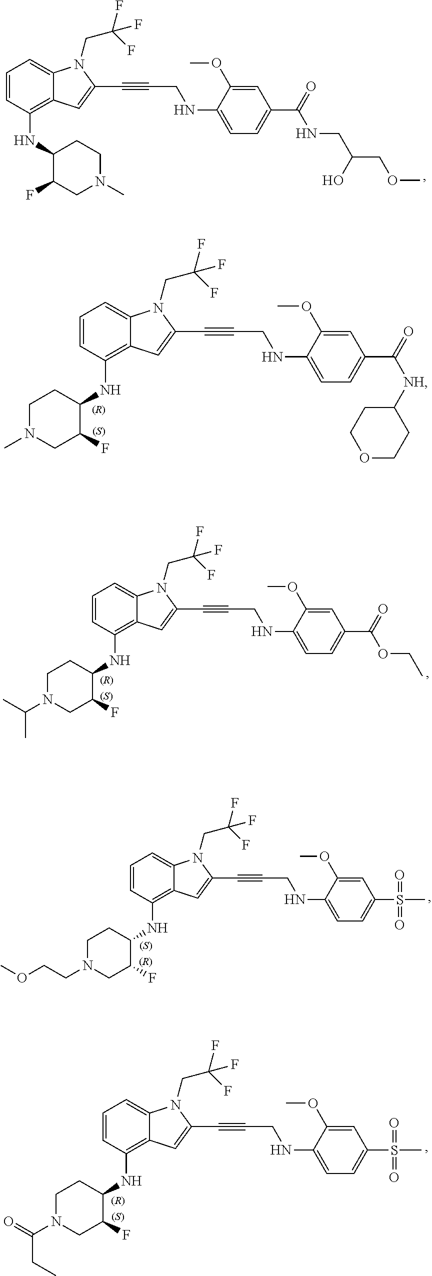

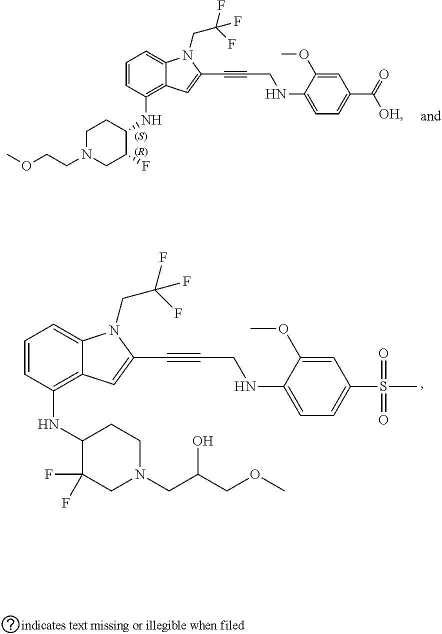

or a pharmaceutically-acceptable salt thereof.

Non-limiting examples of compounds of the current disclosure include the following:

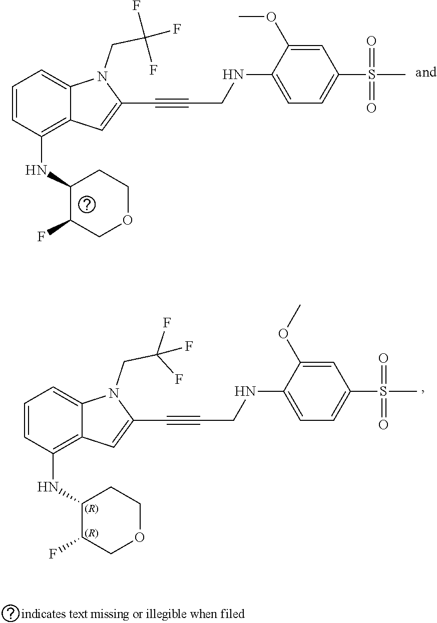

or a pharmaceutically-acceptable salt thereof.

Non-limiting examples of compounds of the current disclosure include the following:

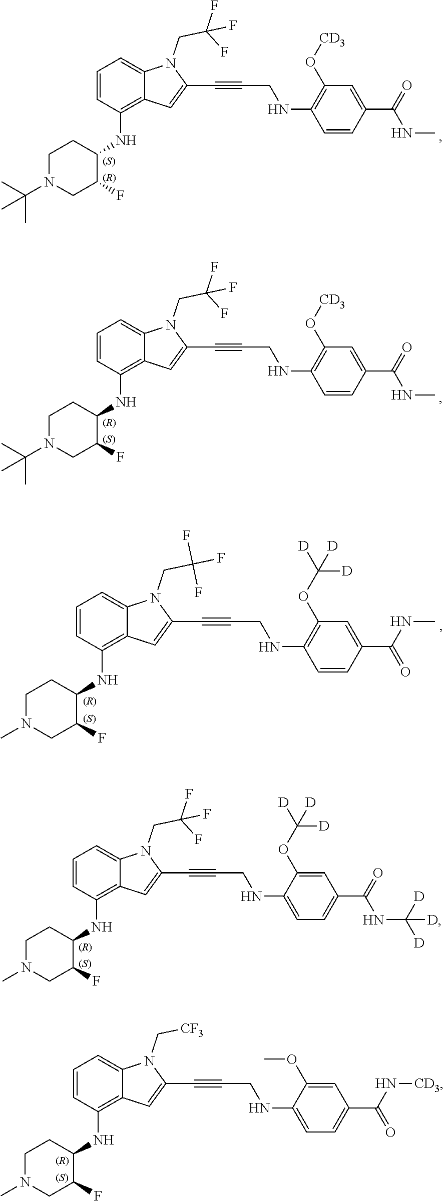

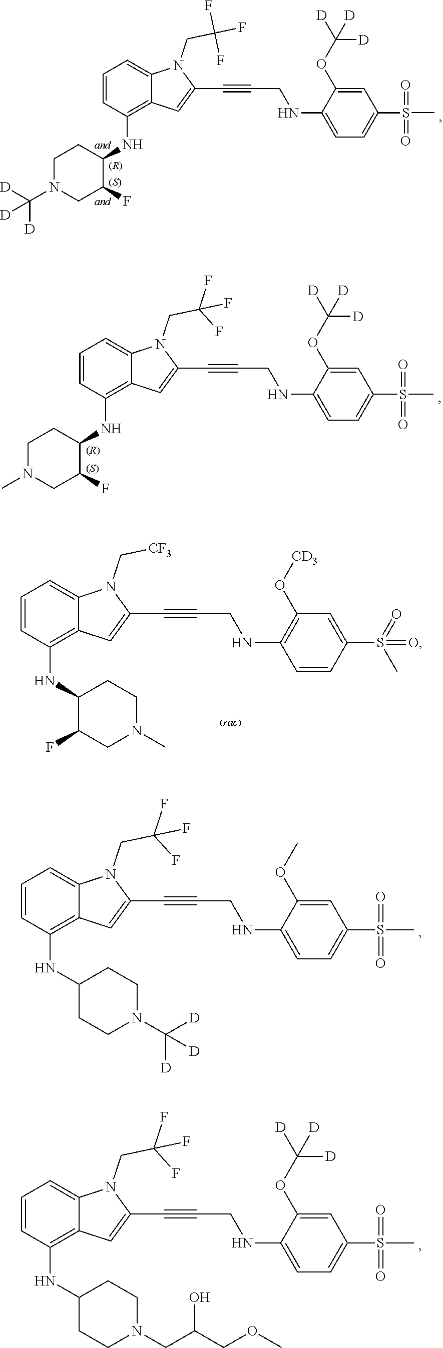

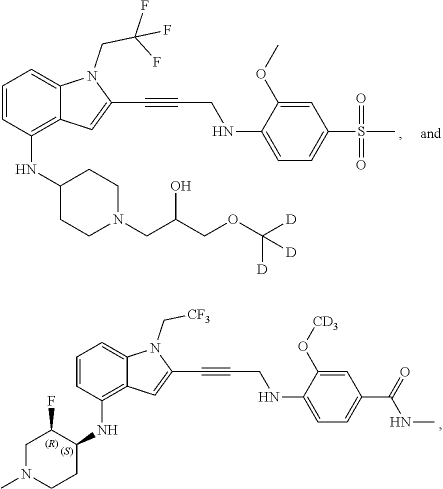

or a pharmaceutically-acceptable salt thereof.

Non-limiting examples of compounds of the current disclosure include the following:

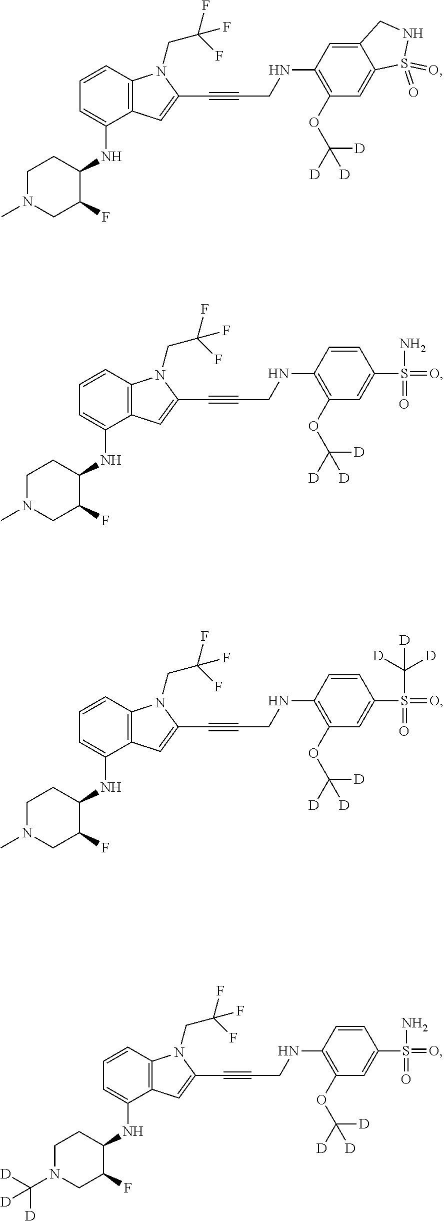 [SMILES: [2H]C([2H])([2H])Oc1cc2c(cc1NCC#Cc1cc3c(N[C@@H]4CCN(C)C[C@@H]4F)cccc3n1CC(F)(F)F)CNS2(=O)=O ; [2H]C([2H])([2H])Oc1cc(S(N)(=O)=O)ccc1NCC#Cc1cc2c(N[C@@H]3CCN(C)C[C@@H]3F)cccc2n1CC(F)(F)F ; [2H]C([2H])([2H])Oc1cc(S(=O)(=O)C([2H])([2H])[2H])ccc1NCC#Cc1cc2c(N[C@@H]3CCN(C)C[C@@H]3F)cccc2n1CC(F)(F)F ; [2H]C([2H])([2H])Oc1cc(S(N)(=O)=O)ccc1NCC#Cc1cc2c(N[C@@H]3CCN(C([2H])([2H])[2H])C[C@@H]3F)cccc2n1CC(F)(F)F]

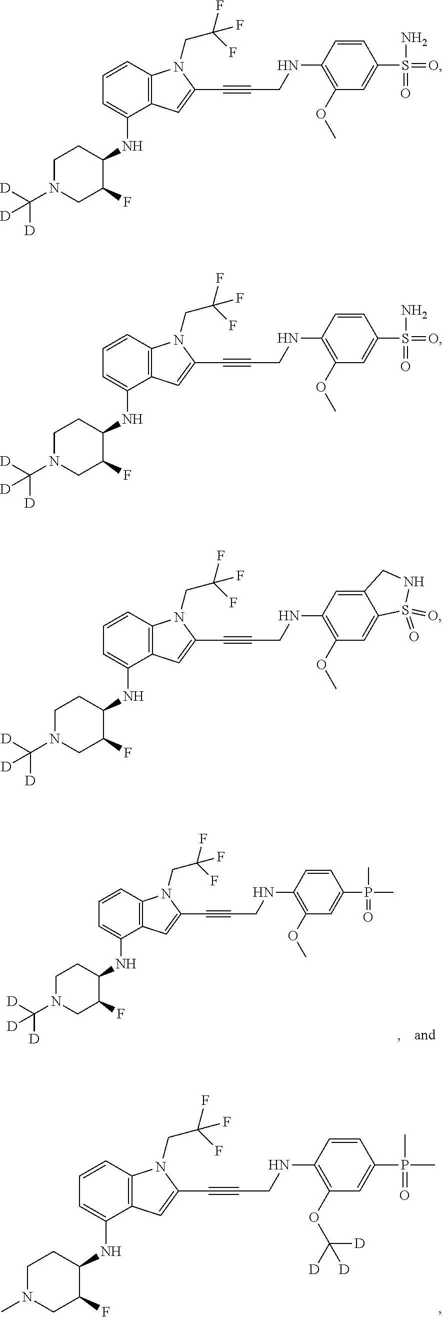

or a pharmaceutically-acceptable salt thereof.

In some embodiments, the disclosure provides a compound comprising: an indole group, wherein the indole group comprises: a) a haloalkyl group at a 1-position of the indole group; b) a first substituent at a 2-position of the indole group, wherein the first substituent is a cyclic group; and c) a second substituent, wherein the second substituent is substituted with at least halo-; or a pharmaceutically-acceptable salt thereof.

In some embodiments, the cyclic group is aryl, heteroaryl, or heterocyclyl, each of which is substituted or unsubstituted. In some embodiments, the cyclic group is unsubstituted aryl. In some embodiments, the cyclic group is substituted aryl. In some embodiments, the cyclic group is substituted phenyl. In some embodiments, the cyclic group is substituted or unsubstituted heteroaryl. In some embodiments, the heteroaryl is an aromatic 5-membered or 6-membered monocyclic ring. In some embodiments, the heteroaryl is thiazolyl, thiadiazolyl, pyrazolyl, thiophenyl, or oxadiazolyl. In some embodiments, the heteroaryl is pyridinyl or pyrimidinyl.

In some embodiments, the second substituent is at a 4-position of the indole group. In some embodiments, the second substituent is a second cyclic group that is substituted or unsubstituted. In some embodiments, the second cyclic group is heterocyclyl. In some embodiments, the heterocyclyl is piperidinyl. In some embodiments, the heterocyclyl is tetrahydropyranyl. In some embodiments, the heterocyclyl is substituted with fluoro-. In some embodiments, the heterocyclyl is substituted with chloro-. In some embodiments, the haloalkyl group is trifluoroethyl.

In some embodiments, the disclosure provides a compound, the compound comprising an indole group, wherein the indole group comprises: a) a substituted or unsubstituted non-cyclic group at a 3-positon of the indole group; and b) a substituted or unsubstituted cyclic group at a 2-position of the indole group, wherein the compound increases a stability of a biologically-active conformation of a p53 mutant relative to a stability of a biologically-active conformation of the p53 mutant in an absence of the compound, or a pharmaceutically-acceptable salt thereof.

In some embodiments, the non-cyclic group is hydrogen. In some embodiments, the non-cyclic group is halo-. In some embodiments, the cyclic group is aryl, heteroaryl, heterocyclyl, or cycloalkylene, each of which is substituted or unsubstituted. In some embodiments, the cyclic group is aryl or heteroaryl, each of which is substituted or unsubstituted. In some embodiments, the cyclic group is substituted aryl. In some embodiments, the cyclic group is substituted phenyl. In some embodiments, the cyclic group is phenyl substituted with alkyl, cycloalkyl, alkoxy, an amine group, a carboxyl group, a carboxylic acid group, a carbamide group, or an amide group, each of which is substituted or unsubstituted; cyano, halo-, or hydrogen.

In some embodiments, the cyclic group is substituted heteroaryl. In some embodiments, the cyclic group is an aromatic 5-membered, 6-membered, 7-membered, or 8-membered monocyclic ring system comprising 1, 2, or 3 heteroatoms as ring members, wherein each heteroatom is independently selected from O, N, or S. In some embodiments, the cyclic group is pyridinyl, pyrimidinyl, thiadiazolyl, thiazolyl, pyrazolyl, thiophenyl, or oxadiazolyl, In some embodiments, the cyclic group is 1,3,5-thiadiazol-2-yl. In some embodiments, the cyclic group is 1,3,4-oxadiazol-2-yl or 1,2,4-oxadiazol-2-yl. In some embodiments, the cyclic group is pyridinyl.

In some embodiments, the indole group further comprises a substituent at a 4-position of the indole group. In some embodiments, the substituent is an amino group that is substituted or unsubstituted. In some embodiments, the amino group is substituted with a second cyclic group. In some embodiments, the second cyclic group is a heterocyclyl group substituted with at least halo-. In some embodiments, the heterocyclyl group is substituted with at least fluoro-. In some embodiments, the heterocyclyl group is substituted with at least chloro-. In some embodiments, the heterocyclyl group is piperidinyl. In some embodiments, the heterocyclyl group is tetrahydropyranyl.

Non-limiting examples of compounds of the disclosure include compounds of any of the following formulae:

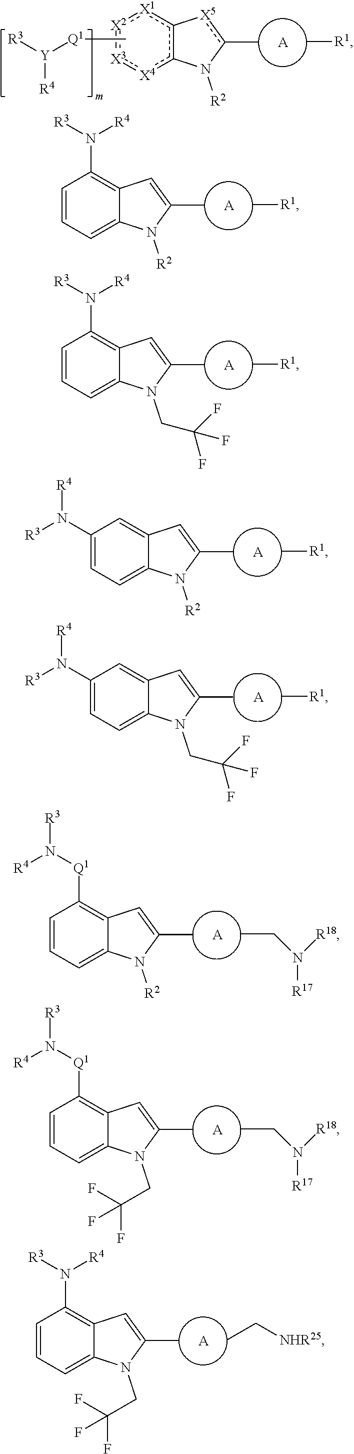

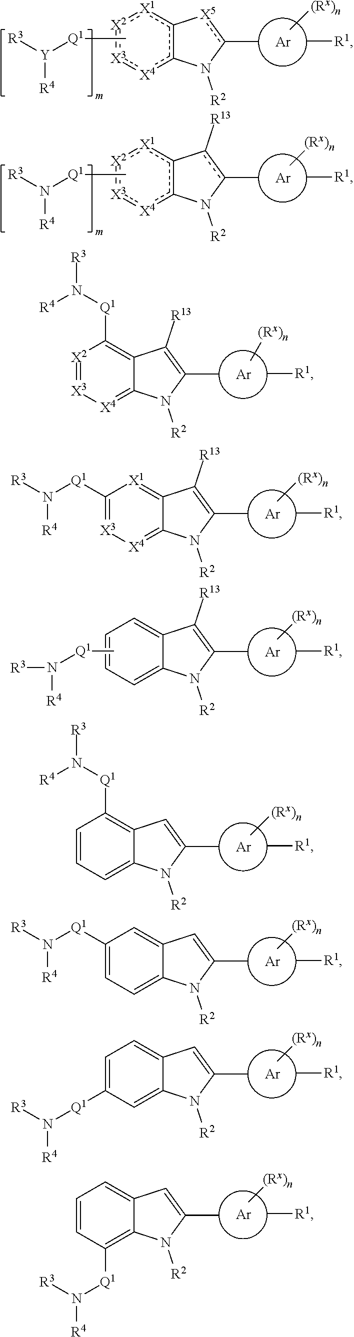

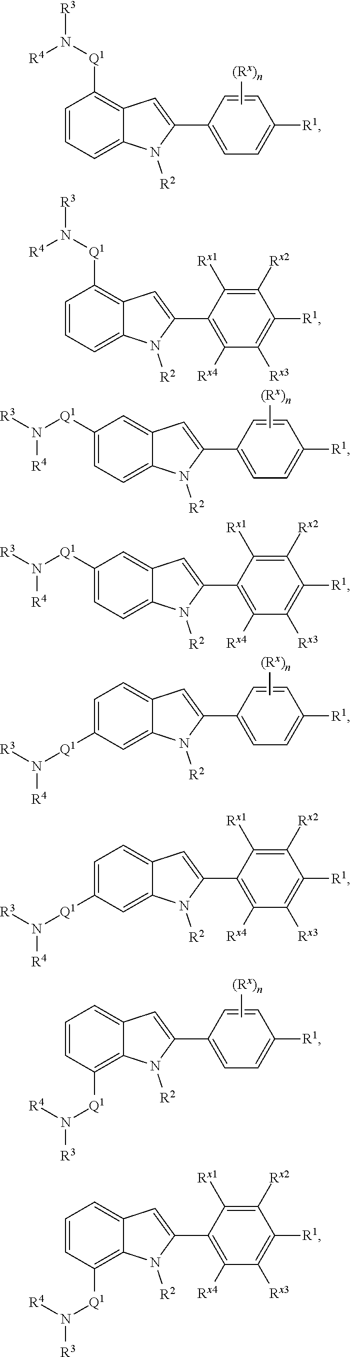

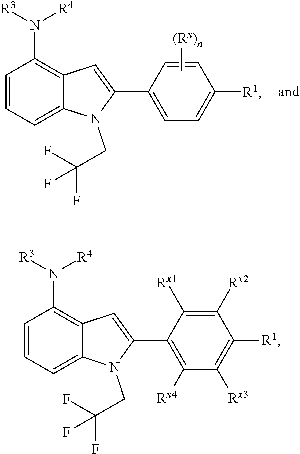

or a pharmaceutically-acceptable salt thereof.

In some embodiments, the disclosure provides a compound of the formula:

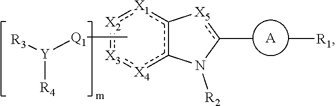

wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
   wherein at least one of X¹, X², X³, and X⁴ is a carbon atom connected to Q¹;
A is a substituted or unsubstituted ring;
Q¹ is alkylene, alkenylene, or alkynylene, each of which is independently substituted or unsubstituted, or C═O, C═S, C═CR¹⁴R¹⁵, C═NR¹⁴, or a bond;
m is 1, 2, 3, or 4;
Y is N, O, or absent;
R¹ is alkyl, alkenyl, alkynyl, alkoxy, aryl, heteroaryl, or heterocyclyl, each of which is independently substituted or unsubstituted, —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, or hydrogen or halogen;
each R³ and R⁴ is independently alkyl, alkenyl, alkynyl, aryl, heteroaryl, or heterocyclyl, each of which is independently substituted or unsubstituted, —C(O)R¹⁹, —C(O)OR¹⁹, —C(O)NR¹⁹R²⁰, —SOR¹⁹, —SO₂R¹⁹, or hydrogen, or R³ and R⁴ together with the nitrogen atom to which R³ and R⁴ are bound form a ring, wherein the ring is substituted or unsubstituted, or R³ is absent;
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently alkyl, alkenyl, alkynyl, aryl, heteroaryl, or heterocyclyl, each of which is independently substituted or unsubstituted, —C(O)R²¹, —C(O)OR²¹, —C(O)NR²¹R²², —OR²¹, —SR²¹, —NR²¹R²², —NR²¹C(O)R²², —OC(O)R²¹, or hydrogen or halogen;
each R¹⁹ and R²⁰ is alkyl, alkenyl, alkynyl, aryl, heteroaryl, or heterocyclyl, each of which is independently substituted or unsubstituted, —C(O)R²³, —C(O)OR²³, —C(O)NR²³R²⁴, —OR²³, —SR²³, —NR²³R²⁴, —NR²³C(O)R²⁴, —OC(O)R²³, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, A is substituted or unsubstituted aryl, heteroaryl, heterocyclyl, cycloalkylene. In some embodiments, A is a 6-carbon monocyclic or 10-carbon bicyclic aromatic ring system wherein 0, 1, 2, 3, or 4 atoms of each ring are optionally substituted. In some embodiments, A is naphthyl. In some embodiments, A is indazolyl.

In some embodiments, A is substituted aryl. In some embodiments, A is substituted phenyl. In some embodiments, A is phenyl substituted with alkyl, cycloalkyl, alkoxy, an amine group, a carboxyl group, a carboxylic acid group, a carbamide group, or an amide group, each of which is substituted or unsubstituted; cyano, halogen, or hydrogen. In some embodiments, A is phenyl substituted with alkyl, wherein alkyl is substituted. In some embodiments, A is phenyl substituted with alkyl, wherein alkyl is substituted with an amino group that is substituted or unsubstituted. In some embodiments, A is phenyl substituted with an amine group that is substituted or unsubstituted. In some embodiments, A is phenyl substituted with a carboxyl group that is substituted or unsubstituted. In some embodiments, A is phenyl substituted with cyano. In some embodiments, A is phenyl substituted with halo-.

In some embodiments, A is substituted or unsubstituted heterocyclyl. In some embodiments, A is substituted heterocyclyl.

In some embodiments, A is an aromatic 5-membered, 6-membered, 7-membered, or 8-membered monocyclic ring system comprising 1, 2, or 3 heteroatoms as ring members, wherein each heteroatom is independently selected from O, N, or S. In some embodiments, A is an aromatic 8-membered, 9-membered, 10-membered, 11-membered, or 12-membered bicyclic ring system comprising 1, 2, 3, 4, 5, or 6 heteroatoms, wherein each heteroatom is independently selected from O, N, or S. In some embodiments, A is an aromatic 5-membered, 6-membered, 7-membered, or 8-membered monocyclic ring system comprising 1, 2, or 3 heteroatoms, and the aromatic 5-membered, 6-membered, 7-membered, or 8-membered monocyclic ring system is substituted. In some embodiments, A is an 8-membered, 9-membered, 10-membered, 11-membered, or 12-membered bicyclic ring system having 1, 2, 3, 4, 5, or 6 heteroatoms, and the 8-membered, 9-membered, 10-membered, 11-membered, or 12-membered bicyclic ring system is substituted.

In some embodiments, A is pyridinyl, pyrimidinyl, thiadiazolyl, thiazolyl, pyrazolyl, thiophenyl, or oxadiazolyl, each of which is independently substituted or unsubstituted. In some embodiments, A is 1,3,5-thiadiazol-2-yl. In some embodiments, A is 1,3,4-oxadiazol-2-yl or 1,2,4-oxadiazol-2-yl. In some embodiments, A is 1,3,4-oxadiazol-2-yl.

In some embodiments, m is 1. In some embodiments, m is 2. In some embodiments, Q¹ is alkylene, alkenylene, or alkynylene, each of which is independently substituted or unsubstituted, or a bond. In some embodiments, Q¹ is a bond. In some embodiments, Y is N.

In some embodiments, R² is hydrogen. In some embodiments, R² is substituted or unsubstituted alkyl. In some embodiments, R² is trifluoroethyl. In some embodiments, R² is cycloalkyl.

In some embodiments, R¹ is alkyl, alkoxy, aryl, heteroaryl, or heterocyclyl, each of which is independently substituted or unsubstituted; —C(O)R¹⁶, —C(O)OR¹⁶, —C(O)NR¹⁶R¹⁷, —OR¹⁶, —NR¹⁶R¹⁷, —NR¹⁶C(O)R¹⁶, —OC(O)R¹⁶, cyano, halo, or halogen. In some embodiments, R¹ is —NR¹⁶R¹⁷. In some embodiments, R¹ is substituted alkyl.

In some embodiments, each R³ and R⁴ is independently aryl, heteroaryl, or heterocyclyl, each of which is independently substituted or unsubstituted; or hydrogen. In some embodiments, R³ is hydrogen, and R⁴ is heterocyclyl substituted at least with halo-. In some embodiments, R⁴ is heterocyclyl substituted with fluoro. In some embodiments, R⁴ is heterocyclyl substituted with chloro.

In some embodiments, R¹³ is alkyl, alkenyl, hydrogen, or halogen. In some embodiments, R¹³ is hydrogen.

In some embodiments, the compound has the formula:

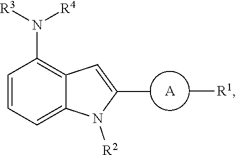

or a pharmaceutically-acceptable salt thereof, wherein the variables are as defined above.

In some embodiments, the compound has the formula:

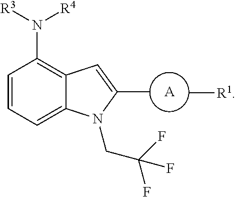

or a pharmaceutically-acceptable salt thereof, wherein the variables are as defined above.

In some embodiments, the compound has the formula:

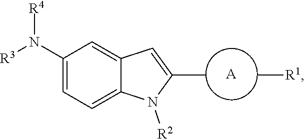

or a pharmaceutically-acceptable salt thereof, wherein the variables are as defined above.

In some embodiments, the compound has the formula:

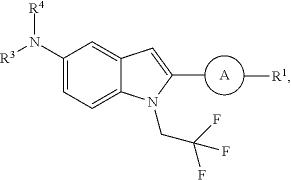

or a pharmaceutically-acceptable salt thereof, wherein the variables are as defined above.

In some embodiments, the disclosure provides a compound of the formula:

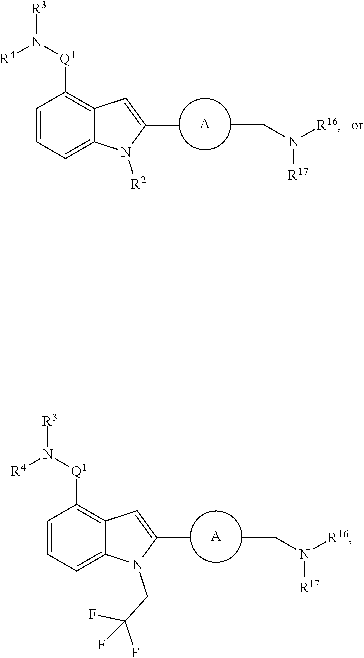

or a pharmaceutically-acceptable salt thereof, wherein the variables are as defined above.

In some embodiments, Q¹ is alkylene, alkenylene, or alkynylene, each of which is independently substituted or unsubstituted, C═O, C═S, C═CR¹⁴R¹⁵, C═NR¹⁴, or a bond. In some embodiments, Q¹ is alkylene, alkenylene, or alkynylene. In some embodiments, Q¹ is C₁-alkylene. In some embodiments, each R¹⁶ and R¹⁷ is independently alkyl, alkenyl, aryl, heteroaryl, heterocyclyl, or hydrogen. In some embodiments, Q¹ is a bond.

In some embodiments, R³ is H, and R⁴ is alkyl, alkenyl, alkynyl, aryl, heteroaryl, or heterocyclyl, each of which is independently substituted or unsubstituted; —C(O)R¹⁹, —C(O)OR¹⁹, —C(O)NR¹⁹R²⁰, —SOR¹⁹, —SO₂R¹⁹, or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R⁴ is a ring that is:

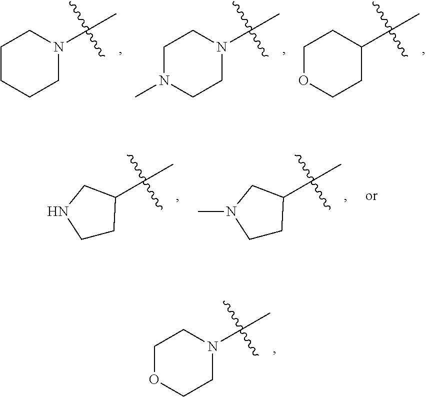

wherein the ring is substituted or unsubstituted. In some embodiments, R³ is H, and R⁴ is a ring that is

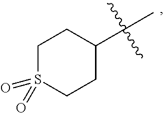 [SMILES: *C1CCS(=O)(=O)CC1]

wherein the ring is substituted or unsubstituted. In some embodiments, R³ is H, and R⁴ is a ring that is

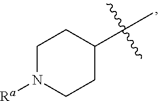

wherein the ring is substituted or unsubstituted. In some embodiments, R^{a} is alkylene. In some embodiments, R^{a} is methyl. In some embodiments, R³ is H, and R⁴ is a ring that is

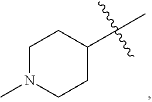 [SMILES: *C1CCN(C)CC1]

wherein the ring is substituted or unsubstituted. In some embodiments, R³ is H, and R⁴ is a ring that is

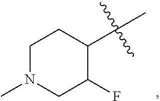 [SMILES: *C1CCN(C)CC1F]

wherein the ring is substituted or unsubstituted.

In some embodiments, each R¹⁶ and R¹⁷ is independently alkyl, cycloalkyl, aryl, heteroaryl, heterocyclyl, alkoxy, carboxyl group, amino group, acyl group, acyloxy group, or an amide group, any of which is unsubstituted or substituted, or hydrogen. In some embodiments, R¹⁶ is hydrogen, and R¹⁷ is a substituted carboxyl group.

In some embodiments, the compound is of the formula:

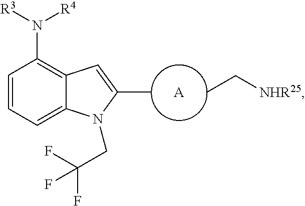

wherein R²⁵ is —C(O)R¹⁶, —C(O)NR¹⁶R¹⁷, alkyl, alkenyl, alkynyl, aryl, heteroaryl, or heterocyclyl, each of which is independently substituted or unsubstituted, or hydrogen. In some embodiments, R²⁵ is aryl that is substituted or unsubstituted. In some embodiments, R²⁵ is substituted phenyl. In some embodiments, R²⁵ is —C(O)R¹⁶, wherein R¹⁶ is alkyl, aryl, heteroaryl, or heterocyclyl. In some embodiments, R²⁵ is —C(O)R¹⁶, wherein R¹⁶ is substituted phenyl.

In some embodiments, the disclosure provides a compound of the formula:

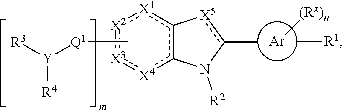

wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
   wherein at least one of X¹, X², X³, and X⁴ is a carbon atom connected to Q¹;
Ar is unsubstituted or substituted aryl;
Q¹ is C═CR¹⁴R¹⁵, C═NR¹⁴, alkylene, alkenylene, or alkynylene, each of which is independently substituted or unsubstituted, C═O, C═S, or a bond;
m is 1, 2, 3, or 4;
n is 0, 1, 2, 3, or 4;
Y is N, O, or absent;
each R^{x} and R¹ is independently C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, or heterocyclyl, each of which is independently substituted or unsubstituted; cyano, halo, or hydrogen; or R¹ and R together with Ar form a fused ring;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

The pattern of dashed bonds can be chosen to provide an aromatic system, for example, an indole, an indolene, a pyrrolopyridine, a pyrrolopyrimidine, or a pyrrolopyrazine. In some embodiments, X¹ is CR⁵, CR⁵R⁶, or a carbon atom connected to Q¹. In some embodiments, X² is CR⁷, CR⁷R⁸, or a carbon atom connected to Q¹. In some embodiments, X³ is CR⁹, CR⁹R¹⁰, or a carbon atom connected to Q¹. In some embodiments, X⁴ is CR¹¹, CR¹¹R¹², or a carbon atom connected to Q¹. In some embodiments, X⁵ is CR¹³, N, or NR¹³. In some embodiments, X¹ is a carbon atom connected to Q¹. In some embodiments, X² is a carbon atom connected to Q¹. In some embodiments, X³ is a carbon atom connected to Q¹. In some embodiments, X⁴ is a carbon atom connected to Q¹. In some embodiments, X⁵ is N.

In some embodiments, Ar is a 6-carbon monocyclic or 10-carbon bicyclic aromatic ring system wherein 0, 1, 2, 3, or 4 atoms of each ring are optionally substituted. In some embodiments, Ar is phenyl. In some embodiments, Ar is naphthyl. In some embodiments, Ar is indazolyl.

R¹ can be —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, or heterocyclyl, each of which is independently substituted or unsubstituted, or hydrogen. In some embodiments, R¹ is alkyl, alkylene, alkoxy, —NR²¹R²², or aryl, each of which is independently substituted or unsubstituted; halo or hydrogen. In some embodiments, R¹ is methyl, cyclohexyl, methylene, methoxy, or benzyl. In some embodiments, R¹ is fluoro or chloro. In some embodiments, R¹ is phenyl. In some embodiments, R¹ is hydrogen.

In some embodiments, R¹ is a substituted alkyl. R¹ can be substituted by one or more substituents selected from a hydroxyl group, sulfhydryl group, halogen, amino group, nitro group, nitroso group, cyano group, azido group, sulfoxide group, sulfone group, sulfonamide group, carboxyl group, carboxaldehyde group, imine group, alkyl group, halo-alkyl group, cyclic alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, urethane group, and ester group.

In some embodiments, R¹ is alkyl substituted with an amine group. In some embodiments, R¹ is methyl substituted with NR¹⁶R¹⁷. In some embodiments, R¹ is alkyl substituted with —C(O)NR¹⁶R¹⁷. In some embodiments, R¹ is methyl substituted with —C(O)NR¹⁶R¹⁷. In some embodiments, R¹ is alkyl substituted with —C(O)OR¹⁶. In some embodiments, R¹ is methyl substituted with COOH.

In some embodiments, m is 1, 2, 3, or 4. In some embodiments, m is 1. In some embodiments, X³ is carbon atom connected to Q¹, and m is 1. In some embodiments, n is 1, 2, or 3. In some embodiments, n is 1. In some embodiments, n is 2. In some embodiments, n is 0.

In some embodiments, Q¹ is C═CR¹⁴R¹⁵, C═NR¹⁴, alkylene, alkenylene, or alkynylene, each of which is independently substituted or unsubstituted, C═O, C═S, or a bond. In some embodiments, Q¹ is alkylene, alkenylene, or alkynylene. In some embodiments, Q¹ is a bond. In some embodiments, Q¹ is C₁-alkylene.

In some embodiments, R² is hydrogen or alkyl. In some embodiments, R¹³ is alkyl, alkenyl, hydrogen, or halogen. In some embodiments, R² is alkyl, and R¹³ is alkyl. In some embodiments, R² is hydrogen, and R¹³ is alkyl. In some embodiments, R² is methyl, ethyl, propyl, iso-propyl, butyl, or tert-butyl. In some embodiments, R¹³ is methyl, ethyl, propyl, iso-propyl, butyl or tert-butyl. In some embodiments, R² is hydrogen, and R¹³ is hydrogen. In some embodiments, R² is trifluoroethyl, and R¹³ is hydrogen.

In some embodiments, R³ is —C(O)R¹⁹, —C(O)OR¹⁹, alkyl, alkenyl, alkynyl, aryl, heteroaryl, or heterocyclyl, each of which is independently substituted or unsubstituted, or hydrogen, and R⁴ is —C(O)R¹⁹, —C(O)OR¹⁹, alkyl, alkenyl, alkynyl, aryl, heteroaryl, or heterocyclyl, each of which is independently substituted or unsubstituted, or hydrogen.

In some embodiments, R³ is H, and R⁴ is —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted; or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R⁴ is a ring that is:

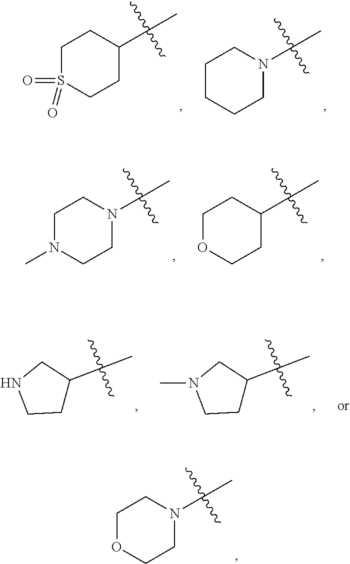

wherein the ring is substituted or unsubstituted. In some embodiments, R³ is H, and R⁴ is a ring that is

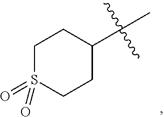 [SMILES: *C1CCS(=O)(=O)CC1]

wherein the ring is substituted or unsubstituted. In some embodiments, R³ is H, and R⁴ is a ring that is

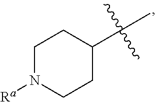

wherein the ring is substituted or unsubstituted. In some embodiments, R^{a} is alkylene. In some embodiments, R^{a} is methyl. In some embodiments, R³ is H, and R⁴ is a ring that is

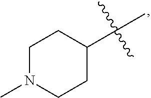 [SMILES: *C1CCN(C)CC1]

wherein the ring is substituted or unsubstituted. In some embodiments, R³ is H, and R⁴ is a ring that is

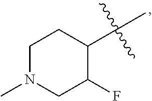 [SMILES: *C1CCN(C)CC1F]

wherein the ring is substituted or unsubstituted. In some embodiments, R³ is H, and R⁴ is a ring that is

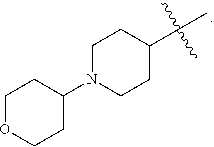 [SMILES: *C1CCN(C2CCOCC2)CC1]

In some embodiments, the disclosure provides a compound of the formula:

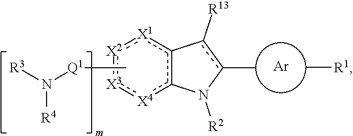

wherein the variables are as defined above.

In some embodiments, the disclosure provides a compound of the formula:

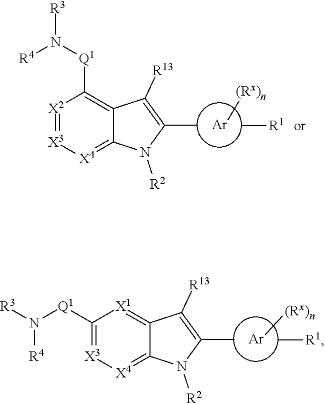

wherein:
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
Ar is unsubstituted or substituted aryl;
Q¹ is C═CR¹⁴R¹⁵, C═NR¹⁴, alkylene, alkenylene, or alkynylene, each of which is independently substituted or unsubstituted, C═O, C═S, or a bond;
n is 0, 1, 2, 3, or 4;
each R^{x} and R¹ is independently C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, or heterocyclyl, each of which is independently substituted or unsubstituted; cyano, halo, or hydrogen; or R¹ and R together with Ar form a fused ring;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, the compound is of the formula:

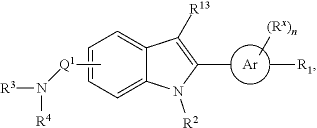

wherein the variables are as defined above.

In some embodiments, Ar is a 6-carbon monocyclic or 10-carbon bicyclic aromatic ring system wherein 0, 1, 2, 3, or 4 atoms of each ring are optionally substituted. In some embodiments, Ar is phenyl. In some embodiments, Ar is naphthyl. In some embodiments, Ar is indazolyl.

In some embodiments, R¹ is a substituted alkyl. R¹ can be substituted by one or more substituents selected from a hydroxyl group, sulfhydryl group, halogen, amino group, nitro group, nitroso group, cyano group, azido group, sulfoxide group, sulfone group, sulfonamide group, carboxyl group, carboxaldehyde group, imine group, alkyl group, halo-alkyl group, cyclic alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, urethane group, and ester group.

In some embodiments, R¹ is alkyl substituted with an amine group. In some embodiments, R¹ is methyl substituted with NR¹⁶R¹⁷. In some embodiments, R¹ is alkyl substituted with —C(O)NR¹⁶R¹⁷. In some embodiments, R¹ is methyl substituted with —C(O)NR¹⁶R¹⁷. In some embodiments, R¹ is alkyl substituted with —C(O)OR¹⁶. In some embodiments, R¹ is methyl substituted with COOH.

In some embodiments, Q¹ is C═CR¹⁴R¹⁵, C═NR¹⁴, alkylene, alkenylene, or alkynylene, each of which is independently substituted or unsubstituted, C═O, C═S, or a bond. In some embodiments, Q¹ is alkylene, alkenylene, or alkynylene. In some embodiments, Q¹ is a bond. In some embodiments, Q¹ is C₁-alkylene.

In some embodiments, R² is hydrogen or alkyl. In some embodiments, R¹³ is alkyl, alkenyl, hydrogen, or halogen. In some embodiments, R² is alkyl, and R¹³ is alkyl. In some embodiments, R² is hydrogen, and R¹³ is alkyl. In some embodiments, R² is methyl, ethyl, propyl, iso-propyl, butyl, or tert-butyl. In some embodiments, R¹³ is methyl, ethyl, propyl, iso-propyl, butyl or tert-butyl. In some embodiments, R² is hydrogen, and R¹³ is hydrogen. In some embodiments, R² is trifluoroethyl, and R¹³ is hydrogen.

In some embodiments, R³ is —C(O)R¹⁹, —C(O)OR¹⁹, alkyl, alkenyl, alkynyl, aryl, heteroaryl, or heterocyclyl, each of which is independently substituted or unsubstituted, or hydrogen, and R⁴ is —C(O)R¹⁹, —C(O)OR¹⁹, alkyl, alkenyl, alkynyl, aryl, heteroaryl, or heterocyclyl, each of which is independently substituted or unsubstituted, or hydrogen.

In some embodiments, R³ is H, and R⁴ is —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted; or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R⁴ is a ring that is:

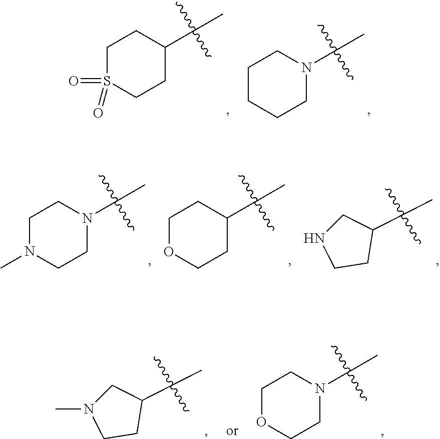

wherein the ring is substituted or unsubstituted. In some embodiments, R³ is H, and R⁴ is a ring that is

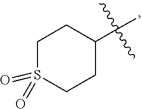

wherein the ring is substituted or unsubstituted. In some embodiments, R³ is H, and R⁴ is a ring that is

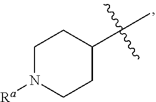

wherein the ring is substituted or unsubstituted. In some embodiments, R^{a} is alkylene. In some embodiments, R^{a} is methyl. In some embodiments, R³ is H, and R⁴ is a ring that is

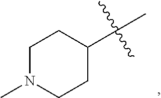 [SMILES: *C1CCN(C)CC1]

wherein the ring is substituted or unsubstituted. In some embodiments, R³ is H, and R⁴ is a ring that is

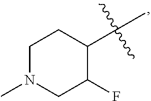 [SMILES: *C1CCN(C)CC1F]

wherein the ring is substituted or unsubstituted. In some embodiments, R³ is H, and R⁴ is a ring that is

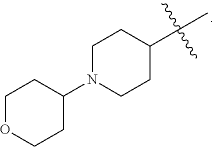 [SMILES: *C1CCN(C2CCOCC2)CC1]

In some embodiments, the disclosure provides a compound of the formula:

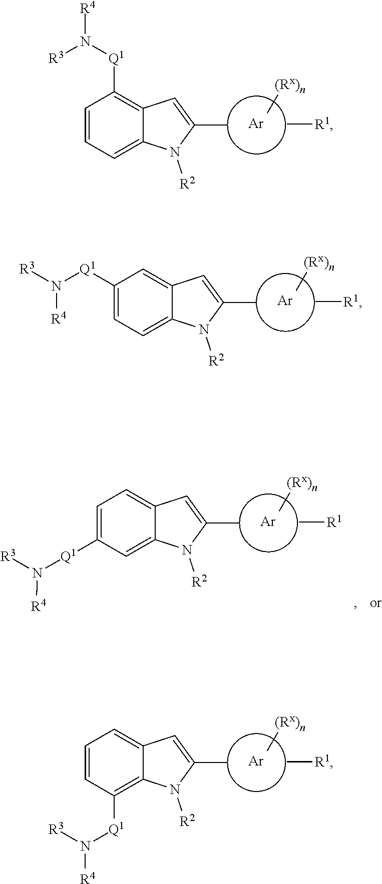

or a pharmaceutically-acceptable salt thereof, wherein the variables are as defined above.

In some embodiments, the disclosure provides a compound of the formula:

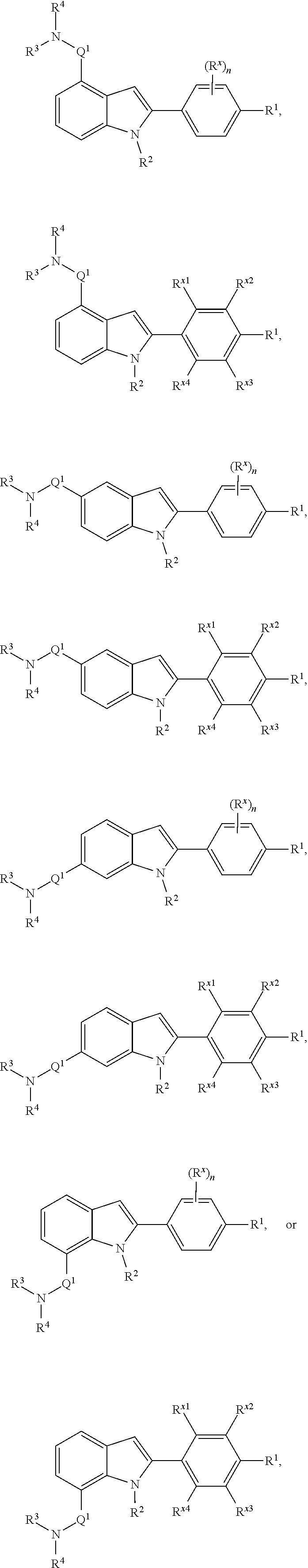

wherein:
Q¹ is C═CR¹⁴R¹⁵, C═NR¹⁴, alkylene, alkenylene, or alkynylene, each of which is independently substituted or unsubstituted, C═O, C═S, or a bond;
each R¹, R^{x}, R^{x1}, R^{x2}, R^{x3}, and R^{x4} is independently —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, or heterocyclyl, each of which is independently substituted or unsubstituted; cyano, halo, or hydrogen; or R¹ and R^{x} together with Ar form a fused ring;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
n is 0, 1, 2, 3, or 4;
each R², R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, R¹ is a substituted alkyl. R¹ can be substituted by one or more substituents selected from a hydroxyl group, sulfhydryl group, halogen, amino group, nitro group, nitroso group, cyano group, azido group, sulfoxide group, sulfone group, sulfonamide group, carboxyl group, carboxaldehyde group, imine group, alkyl group, halo-alkyl group, cyclic alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, urethane group, and ester group.

In some embodiments, R¹ is alkyl substituted with an amine group. In some embodiments, R¹ is methyl substituted with NR¹⁶R¹⁷. In some embodiments, R¹ is methyl substituted with NR¹⁶R¹⁷, wherein R¹⁶ is hydrogen, and R¹⁷ is alkyl, aryl, heteroaryl, an amino group, a carboxyl group, or an ester group, any of which is substituted or unsubstituted. In some embodiments, R¹ is methyl substituted with NR¹⁶R¹⁷, wherein R¹⁶ is hydrogen, and R¹⁷ is substituted or unsubstituted alkyl, aryl, or heteroaryl. In some embodiments, R¹ is methyl substituted with NR¹⁶R¹⁷, wherein R¹⁶ is hydrogen, and R¹⁷ is substituted or unsubstituted phenyl. In some embodiments, R¹ is methyl substituted with NR¹⁶R¹⁷, wherein R¹⁶ is hydrogen, and R¹⁷ is substituted or unsubstituted pyridinyl.

In some embodiments, R¹ is —C(O)NR¹⁶R¹⁷. In some embodiments, R¹ is —C(O)NR¹⁶R¹⁷, wherein R¹⁶ and R¹⁷ are hydrogen. In some embodiments, R¹ is —C(O)NR¹⁶R¹⁷, wherein R¹⁶ is hydrogen, and R¹⁷ alkyl. In some embodiments, R¹ is —C(O)NR¹⁶R¹⁷, wherein R¹⁶ is hydrogen, and R¹⁷ methyl. In some embodiments, R¹ is —C(O)OR¹⁶. In some embodiments, R¹ is —C(O)OH. In some embodiments, R¹ is methyl. In some embodiments, R¹ is halogen. In some embodiments, R¹ is chloro or fluoro.

In some embodiments, n is 0, 1, 2, or 3. In some embodiments, n is 1. In some embodiments, n is 2. In some embodiments, n is 0.

In some embodiments, Q¹ is C═CR¹⁴R¹⁵, C═NR¹⁴, alkylene, alkenylene, or alkynylene, each of which is independently substituted or unsubstituted, C═O, C═S, or a bond. In some embodiments, Q¹ is alkylene, alkenylene, or alkynylene. In some embodiments, Q¹ is a bond. In some embodiments, Q¹ is C₁-alkylene.

In some embodiments, R² is hydrogen or alkyl. In some embodiments, R¹³ is alkyl, alkenyl, hydrogen, or halogen. In some embodiments, R² is alkyl, and R¹³ is alkyl. In some embodiments, R² is hydrogen, and R¹³ is alkyl. In some embodiments, R² is methyl, ethyl, propyl, iso-propyl, butyl, or tert-butyl. In some embodiments, R¹³ is methyl, ethyl, propyl, iso-propyl, butyl or tert-butyl. In some embodiments, R² is hydrogen, and R¹³ is hydrogen. In some embodiments, R² is trifluoroethyl, and R¹³ is hydrogen.

In some embodiments, R³ is —C(O)R¹⁹, —C(O)OR¹⁹, alkyl, alkenyl, alkynyl, aryl, heteroaryl, or heterocyclyl, each of which is independently substituted or unsubstituted, or hydrogen, and R⁴ is —C(O)R¹⁹, —C(O)OR¹⁹, alkyl, alkenyl, alkynyl, aryl, heteroaryl, or heterocyclyl, each of which is independently substituted or unsubstituted, or hydrogen.

In some embodiments, R³ is H, and R⁴ is —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted; or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R⁴ is a ring that is:

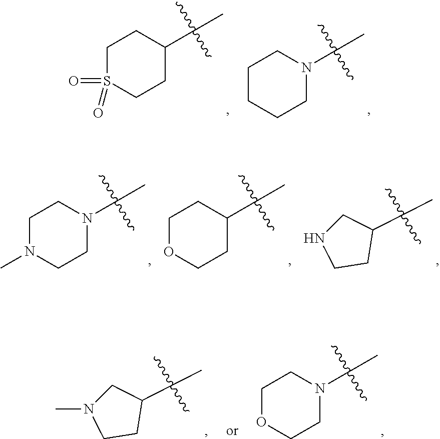

wherein the ring is substituted or unsubstituted. In some embodiments, R³ is H, and R⁴ is a ring that is

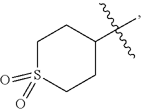

wherein the ring is substituted or unsubstituted. In some embodiments, R³ is H, and R⁴ is a ring that is

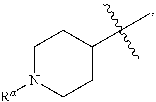

wherein the ring is substituted or unsubstituted. In some embodiments, R^{a} is alkylene. In some embodiments, R^{a} is methyl. In some embodiments, R³ is H, and R⁴ is a ring that is

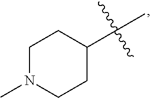 [SMILES: *C1CCN(C)CC1]

wherein the ring is substituted or unsubstituted. In some embodiments, R³ is H, and R⁴ is a ring that is

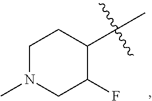 [SMILES: *C1CCN(C)CC1F]

wherein the ring is substituted or unsubstituted. In some embodiments, R³ is H, and R⁴ is a ring that is

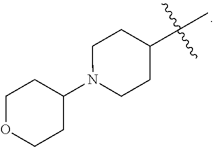 [SMILES: *C1CCN(C2CCOCC2)CC1]

In some embodiments, the disclosure provides a compound of the formula:

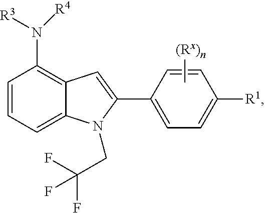

or a pharmaceutically-acceptable salt thereof, wherein the variables are as defined above.

In some embodiments, R¹ is a substituted alkyl. R¹ can be substituted by one or more substituents selected from a hydroxyl group, sulfhydryl group, halogen, amino group, nitro group, nitroso group, cyano group, azido group, sulfoxide group, sulfone group, sulfonamide group, carboxyl group, carboxaldehyde group, imine group, alkyl group, halo-alkyl group, cyclic alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, urethane group, and ester group.

In some embodiments, R¹ is alkyl substituted with an amine group. In some embodiments, R¹ is methyl substituted with NR¹⁶R¹⁷. In some embodiments, R¹ is methyl substituted with NR¹⁶R¹⁷, wherein R¹⁶ is hydrogen, and R¹⁷ is alkyl, aryl, heteroaryl, an amino group, a carboxyl group, or an ester group, any of which is substituted or unsubstituted. In some embodiments, R¹ is methyl substituted with NR¹⁶R¹⁷, wherein R¹⁶ is hydrogen, and R¹⁷ is substituted or unsubstituted alkyl, aryl, or heteroaryl. In some embodiments, R¹ is methyl substituted with NR¹⁶R¹⁷, wherein R¹⁶ is hydrogen, and R¹⁷ is substituted or unsubstituted phenyl. In some embodiments, R¹ is methyl substituted with NR¹⁶R¹⁷, wherein R¹⁶ is hydrogen, and R¹⁷ is substituted or unsubstituted pyridinyl.

In some embodiments, R¹ is —C(O)NR¹⁶R¹⁷. In some embodiments, R¹ is —C(O)NR¹⁶R¹⁷, wherein R¹⁶ and R¹⁷ are hydrogen. In some embodiments, R¹ is —C(O)NR¹⁶R¹⁷, wherein R¹⁶ is hydrogen, and R¹⁷ alkyl. In some embodiments, R¹ is —C(O)NR¹⁶R¹⁷, wherein R¹⁶ is hydrogen, and R¹⁷ methyl. In some embodiments, R¹ is —C(O)OR¹⁶. In some embodiments, R¹ is —C(O)OH. In some embodiments, R¹ is methyl. In some embodiments, R¹ is halogen. In some embodiments, R¹ is chloro or fluoro.

In some embodiments, n is 1, 2, or 3. In some embodiments, n is 1. In some embodiments, n is 2. In some embodiments, n is 0.

In some embodiments, R³ is —C(O)R¹⁹, —C(O)OR¹⁹, alkyl, alkenyl, alkynyl, aryl, heteroaryl, or heterocyclyl, each of which is independently substituted or unsubstituted, or hydrogen, and R⁴ is —C(O)R¹⁹, —C(O)OR¹⁹, alkyl, alkenyl, alkynyl, aryl, heteroaryl, or heterocyclyl, each of which is independently substituted or unsubstituted, or hydrogen.

In some embodiments, R³ is H, and R⁴ is —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted; or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R³ is H, and R⁴ is a ring that is:

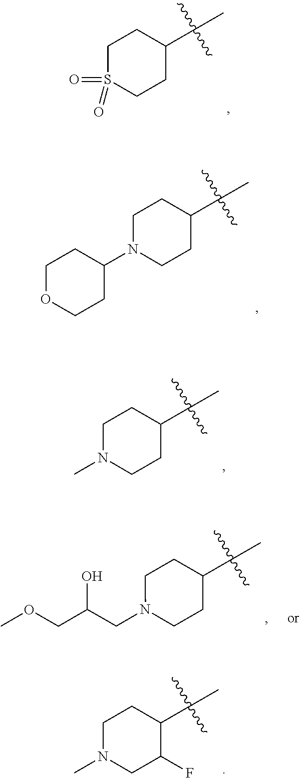

In some embodiments, R³ is H, and R⁴ is a ring that is

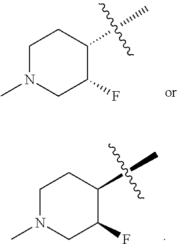

In some embodiments, R³ is H, and R⁴ is a ring that is

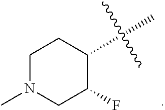

Non-limiting examples of compounds of the disclosure include compounds of any of the following formulae:

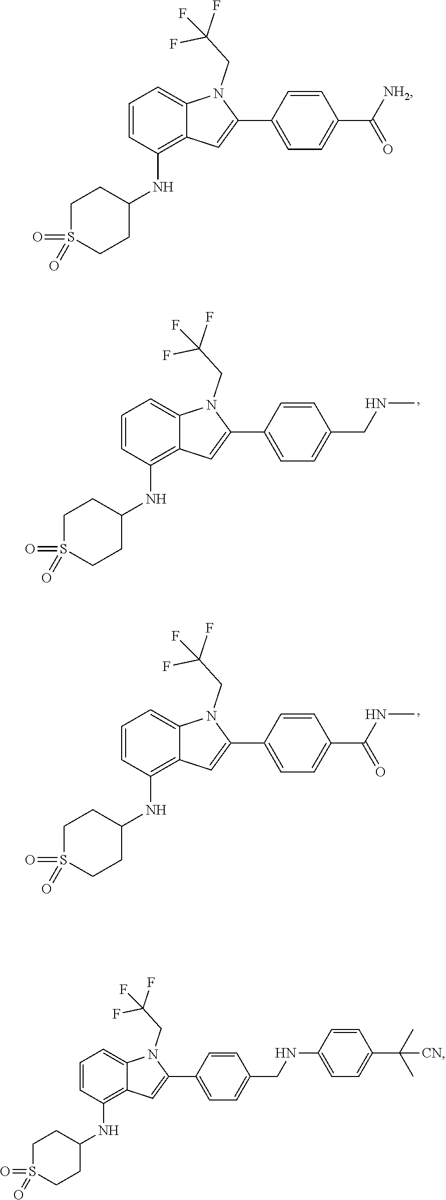 [SMILES: NC(=O)c1ccc(-c2cc3c(NC4CCS(=O)(=O)CC4)cccc3n2CC(F)(F)F)cc1 ; CNCc1ccc(-c2cc3c(NC4CCS(=O)(=O)CC4)cccc3n2CC(F)(F)F)cc1 ; CNC(=O)c1ccc(-c2cc3c(NC4CCS(=O)(=O)CC4)cccc3n2CC(F)(F)F)cc1 ; CC(C)(C#N)c1ccc(NCc2ccc(-c3cc4c(NC5CCS(=O)(=O)CC5)cccc4n3CC(F)(F)F)cc2)cc1]

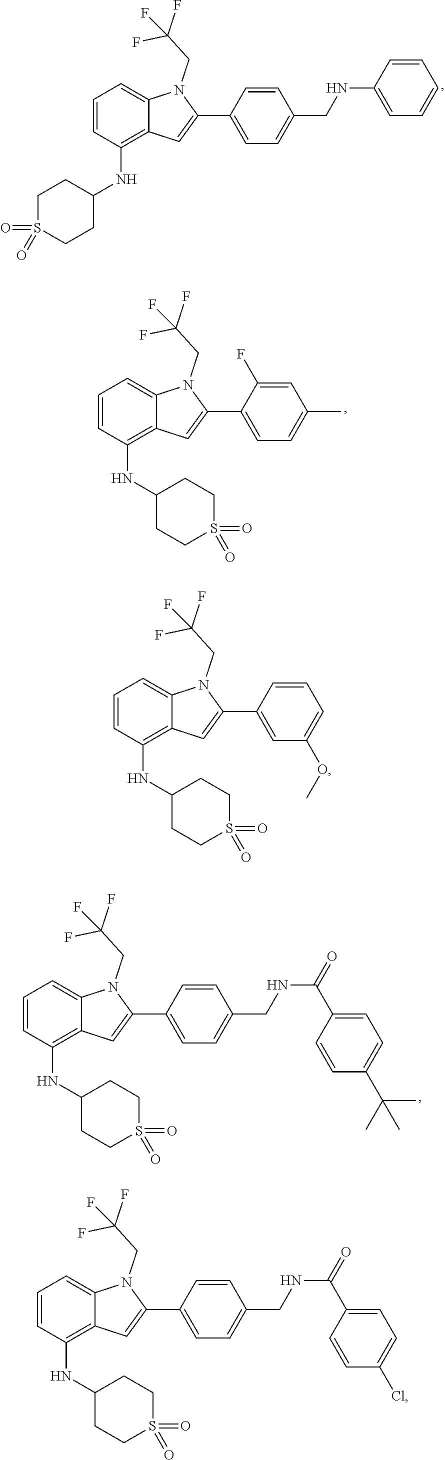

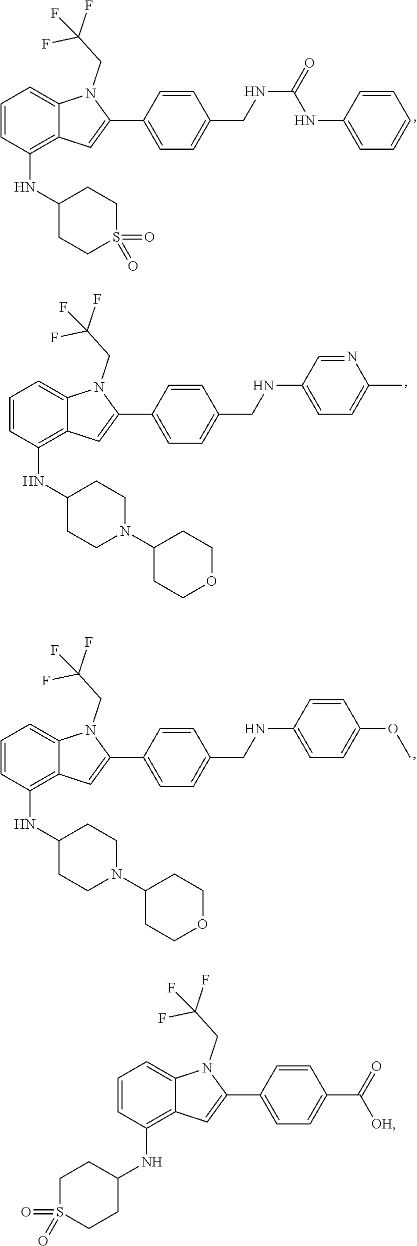

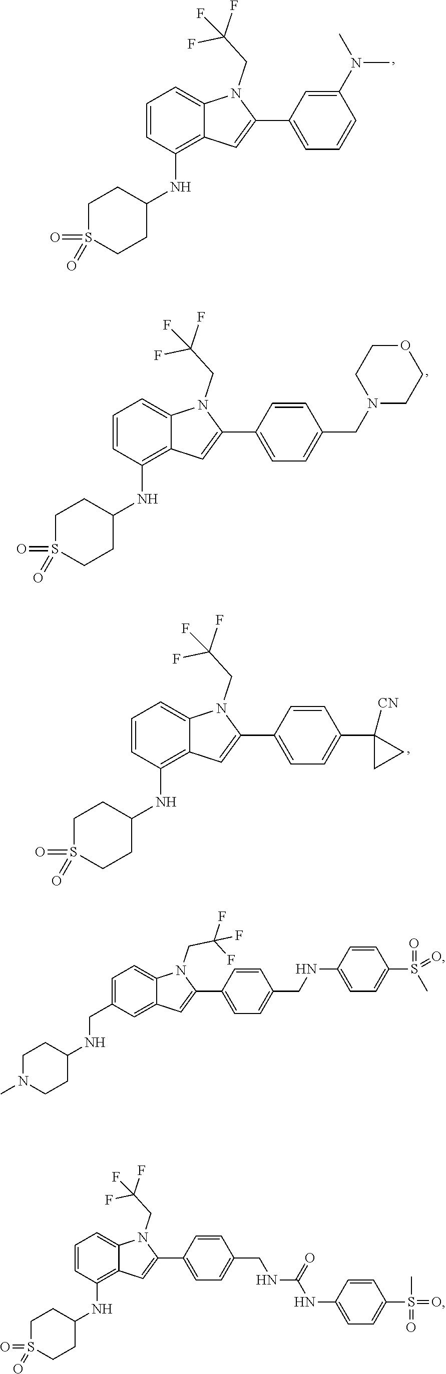

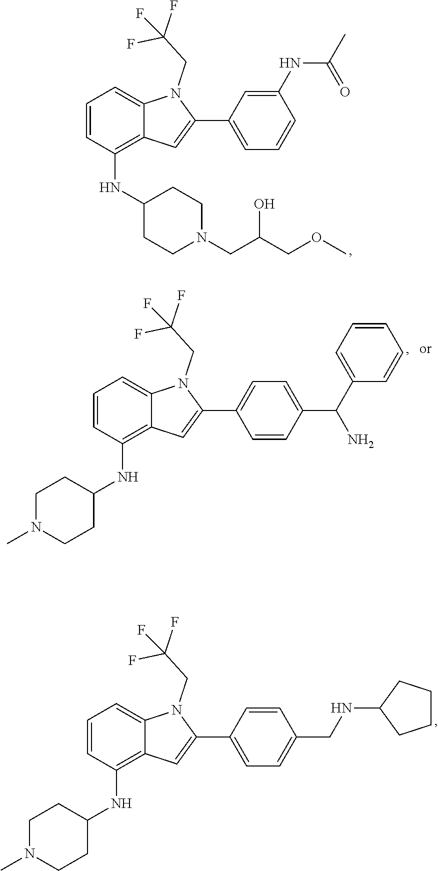

or a pharmaceutically-acceptable salt thereof.

In some embodiments, the disclosure provides a compound of the formula:

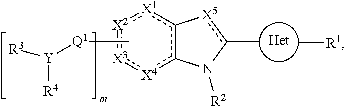

wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
   wherein at least one of X¹, X², X³, and X⁴ is a carbon atom connected to Q¹;
Het is substituted or unsubstituted heteroaryl;
Q¹ is alkylene, alkenylene, or alkynylene, each of which is independently substituted or unsubstituted, or C═O, C═S, C═CR¹⁴R¹⁵, C═NR¹⁴, or a bond;
m is 1, 2, 3, or 4;
Y is N, O, or absent;
R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, or heterocyclyl, each of which is independently substituted or unsubstituted, or hydrogen or halogen;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
each R², R⁵, R⁶, R⁷, R⁸, R⁹, R¹⁰, R¹¹, R¹², R¹³, R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

The pattern of dashed bonds can be chosen to provide an aromatic system, for example, an indole, an indolene, a pyrrolopyridine, a pyrrolopyrimidine, or a pyrrolopyrazine. In some embodiments, X¹ is CR⁵, CR⁵R⁶, or a carbon atom connected to Q¹. In some embodiments, X² is CR⁷, CR⁷R⁸, or a carbon atom connected to Q¹. In some embodiments, X³ is CR⁹, CR⁹R¹⁰, or a carbon atom connected to Q¹. In some embodiments, X⁴ is CR¹¹, CR¹¹R¹², or a carbon atom connected to Q¹. In some embodiments, X⁵ is CR¹³, N, or NR¹³. In some embodiments, X¹ is a carbon atom connected to Q¹. In some embodiments, X² is a carbon atom connected to Q¹. In some embodiments, X³ is a carbon atom connected to Q¹. In some embodiments, X⁴ is a carbon atom connected to Q¹. In some embodiments, X⁵ is N.

In some embodiments, Het is an aromatic 5-membered, 6-membered, 7-membered, or 8-membered monocyclic ring system comprising 1, 2, or 3 heteroatoms as ring members, wherein each heteroatom is independently selected from O, N, or S. In some embodiments, Het is an aromatic 8-membered, 9-membered, 10-membered, 11-membered, or 12-membered bicyclic ring system comprising 1, 2, 3, 4, 5, or 6 heteroatoms, wherein each heteroatom is independently selected from O, N, or S. In some embodiments, Het is an aromatic 5-membered, 6-membered, 7-membered, or 8-membered monocyclic ring system comprising 1, 2, or 3 heteroatoms, and the aromatic 5-membered, 6-membered, 7-membered, or 8-membered monocyclic ring system is substituted. In some embodiments, Het is an 8-membered, 9-membered, 10-membered, 11-membered, or 12-membered bicyclic ring system having 1, 2, 3, 4, 5, or 6 heteroatoms, and the 8-membered, 9-membered, 10-membered, 11-membered, or 12-membered bicyclic ring system is substituted.

In some embodiments, Het is pyridinyl, pyrimidinyl, thiadiazolyl, thiazolyl, pyrazolyl, thiophenyl, or oxadiazolyl, each of which is independently substituted or unsubstituted. In some embodiments, Het is 1,3,5-thiadiazol-2-yl. In some embodiments, Het is 1,3,4-oxadiazol-2-yl or 1,2,4-oxadiazol-2-yl. In some embodiments, Het is 1,3,4-oxadiazol-2-yl.

In some embodiments, R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, or heterocyclyl, each of which is independently substituted or unsubstituted, or hydrogen. In some embodiments, R¹ is alkyl, alkylene, alkoxy, —NR²¹R²², or aryl, each of which is independently substituted or unsubstituted; halo or hydrogen. In some embodiments, R¹ is methyl, cyclohexyl, methylene, methoxy, or benzyl. In some embodiments, R¹ is fluoro or chloro. In some embodiments, R¹ is phenyl. In some embodiments, R¹ is hydrogen.

In some embodiments, R¹ is a substituted alkyl or alkylene. R¹ can be substituted by one or more substituents selected from a hydroxyl group, sulfhydryl group, halogen, amino group, nitro group, nitroso group, cyano group, azido group, sulfoxide group, sulfone group, sulfonamide group, carboxyl group, carboxaldehyde group, imine group, alkyl group, halo-alkyl group, cyclic alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, urethane group, and ester group.

In some embodiments, R¹ is substituted alkyl. In some embodiments, R¹ is alkyl substituted with NR¹⁶R¹⁷. In some embodiments, R¹ is methyl substituted with NR¹⁶R¹⁷, wherein each R¹⁶ and R¹⁷ is independently alkyl, cycloalkyl, aryl, heteroaryl, heterocyclyl, alkoxy, carboxyl group, amino group, acyl group, acyloxy group, or an amide group, any of which is unsubstituted or substituted, or hydrogen. In some embodiments, R¹ is methyl substituted with NR¹⁶R¹⁷, wherein R¹⁶ is hydrogen, and R¹⁷ is a substituted carboxyl group.

In some embodiments, m is 1, 2, 3, or 4. In some embodiments, m is 1. In some embodiments, X¹ is carbon atom connected to Q¹, and m is 1. In some embodiments, X² is carbon atom connected to Q¹, and m is 1.

In some embodiments, Q¹ is C═CR¹⁴R¹⁵, C═NR¹⁴, alkylene, alkenylene, or alkynylene, each of which is independently substituted or unsubstituted, or C═O, C═S, or a bond. In some embodiments, Q¹ is alkylene, alkenylene, or alkynylene. In some embodiments, Q¹ is C₁-alkylene. In some embodiments, each R¹⁶ and R¹⁷ is independently alkyl, alkenyl, aryl, heteroaryl, heterocyclyl, or hydrogen. In some embodiments, Q¹ is a bond.

In some embodiments, Q¹ is C₁-alkylene, R¹⁶ is aryl, and R¹⁷ is alkyl. In some embodiments, Q¹ is C₁-alkylene, R¹⁶ is aryl, and R¹⁷ is hydrogen. In some embodiments, Q¹ is C₁-alkylene, R¹⁶ is heteroaryl, and R¹⁷ is alkyl. In some embodiments, Q¹ is C₁-alkylene, R¹⁶ is heteroaryl, and R¹⁷ is hydrogen. In some embodiments, Q¹ is C₁-alkylene, R¹⁶ is substituted heteroaryl, and R¹⁷ is hydrogen. In some embodiments, Q¹ is C₁-alkylene, R¹⁶ is substituted alkyl, and R¹⁷ is hydrogen. In some embodiments, R¹⁷ is aryl, heteroaryl, or heterocyclyl, each of which is independently substituted or unsubstituted with halogen, alkyl, or hydroxyl. In some embodiments, R¹⁶ is hydrogen, and R¹⁷ is aryl or heteroaryl, substituted or unsubstituted with halogen or alkyl. In some embodiments, R¹⁶ is alkyl, and R¹⁷ is heteroaryl substituted with halogen or alkyl. In some embodiments, R¹⁷ is aryl, heteroaryl, or heterocyclyl, each of which is independently substituted or unsubstituted with alkyl. In some embodiments, R¹⁷ is aryl or heteroaryl, each of which is independently substituted with alkyl, wherein the alkyl is optionally substituted with fluorine, chlorine, bromine, iodine, or cyano.

In some embodiments, R² is hydrogen or alkyl. In some embodiments, R² is substituted alkyl. In some embodiments, R² is trifluoroethyl. In some embodiments, R¹³ is alkyl, alkenyl, hydrogen, or halogen. In some embodiments, R¹³ is methyl, ethyl, propyl, iso-propyl, butyl or tert-butyl. In some embodiments, R² is trifluoroethyl, and R¹³ is hydrogen.

In some embodiments, R³ is —C(O)R¹⁹, —C(O)OR¹⁹, alkyl, alkenyl, alkynyl, aryl, heteroaryl, or heterocyclyl, each of which is independently substituted or unsubstituted, or hydrogen; and R⁴ is —C(O)R¹⁹, —C(O)OR¹⁹, alkyl, alkenyl, alkynyl, aryl, heteroaryl, or heterocyclyl, each of which is independently substituted or unsubstituted, or hydrogen.

In some embodiments, R³ is H, and R⁴ is —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted; or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R⁴ is a ring that is

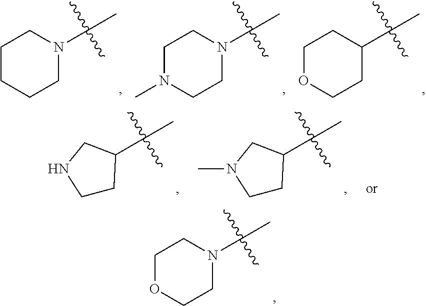

wherein the ring is substituted or unsubstituted. In some embodiments, R³ is H, and R⁴ is a ring that is

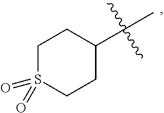 [SMILES: *C1CCS(=O)(=O)CC1]

wherein the ring is substituted or unsubstituted. In some embodiments, R³ is H, and R⁴ is a ring that is

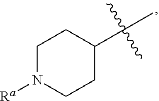

wherein the ring is substituted or unsubstituted. In some embodiments, R^{a} is alkylene. In some embodiments, R^{a} is methyl. In some embodiments, R³ is H, and R⁴ is a ring that is

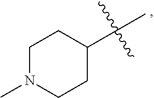 [SMILES: *C1CCN(C)CC1]

wherein the ring is substituted or unsubstituted. In some embodiments, R³ is H, and R⁴ is a ring that is

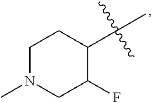 [SMILES: *C1CCN(C)CC1F]

wherein the ring is substituted or unsubstituted.

In some embodiments, R³ and R⁴ together with the nitrogen atom to which R³ and R⁴ are bound form a ring, wherein the ring is substituted or unsubstituted. In some embodiments, R³ and R⁴ together with the nitrogen atom to which R³ and R⁴ are bound form a substituted heterocycle. In some embodiments, R³ and R⁴ together with the nitrogen atom to which R³ and R⁴ are bound form a heterocycle substituted with a hydroxyl group, halogen, amino group, or alkyl group. In some embodiments, R³ and R⁴ together with the nitrogen atom to which R³ and R⁴ are bound form a heterocycle, wherein the heterocycle is substituted by a substituted or unsubstituted heterocycle.

In some embodiments, the disclosure provides a compound of the formula:

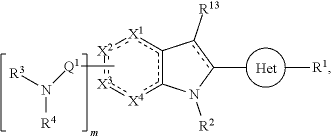

or a pharmaceutically-acceptable salt thereof, wherein the variables are as defined above.

In some embodiments, the disclosure provides a compound of the formula:

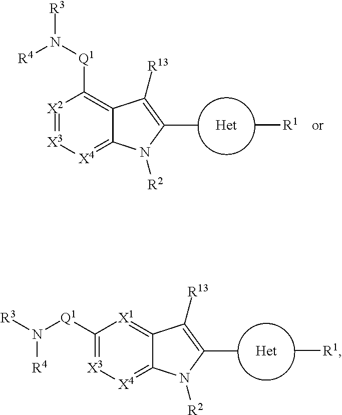

or a pharmaceutically-acceptable salt thereof, wherein the variables are as defined above.

In some embodiments, the compound is of the formula:

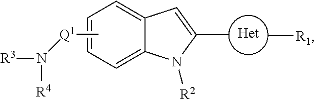

or a pharmaceutically-acceptable salt thereof, wherein the variables are as defined above.

In some embodiments, the disclosure provides a compound of the formula:

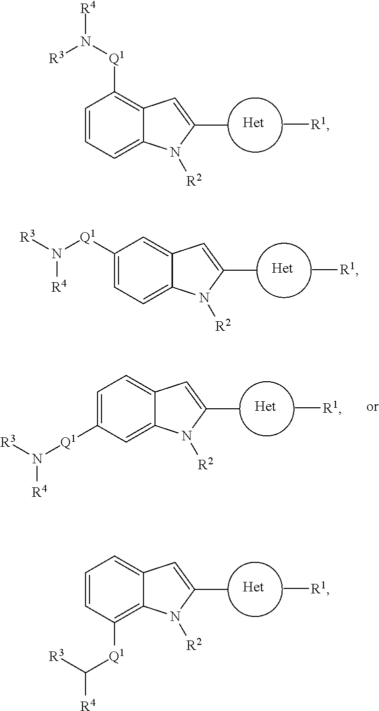

or a pharmaceutically-acceptable salt thereof, wherein the variables are as defined above.

In some embodiments, R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, or heterocyclyl, each of which is independently substituted or unsubstituted, or hydrogen. In some embodiments, R¹ is alkyl, alkylene, alkoxy, —NR²¹R²², or aryl, each of which is independently substituted or unsubstituted; halo or hydrogen.

In some embodiments, R¹ is substituted alkyl. In some embodiments, R¹ is alkyl substituted with NR¹⁶R¹⁷. In some embodiments, R¹ is methyl substituted with NR¹⁶R¹⁷, wherein each R¹⁶ and R¹⁷ is independently alkyl, cycloalkyl, aryl, heteroaryl, heterocyclyl, alkoxy, carboxyl group, amino group, acyl group, acyloxy group, or an amide group, any of which is unsubstituted or substituted, or hydrogen. In some embodiments, R¹ is methyl substituted with NR¹⁶R¹⁷, wherein R¹⁶ is hydrogen, and R¹⁷ is a substituted carboxyl group.

In some embodiments, R² is hydrogen or alkyl. In some embodiments, R² is substituted alkyl. In some embodiments, R² is trifluoroethyl.

In some embodiments, Q¹ is C═CR¹⁴R¹⁵, C═NR¹⁴, alkylene, alkenylene, or alkynylene, each of which is independently substituted or unsubstituted, C═O, C═S, or a bond. In some embodiments, Q¹ is alkylene, alkenylene, or alkynylene. In some embodiments, Q¹ is C₁-alkylene. In some embodiments, each R¹⁶ and R¹⁷ is independently alkyl, alkenyl, aryl, heteroaryl, heterocyclyl, or hydrogen. In some embodiments, Q¹ is a bond.

In some embodiments, R³ is H, and R⁴ is —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted; or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R⁴ is a ring that is:

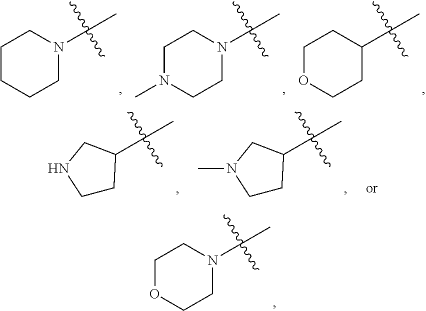

wherein the ring is substituted or unsubstituted. In some embodiments, R³ is H, and R⁴ is a ring that is

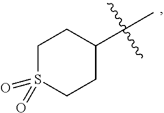 [SMILES: *C1CCS(=O)(=O)CC1]

wherein the ring is substituted or unsubstituted. In some embodiments, R³ is H, and R⁴ is a ring that is

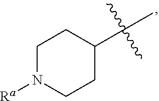

wherein the ring is substituted or unsubstituted. In some embodiments, R^{a} is alkylene. In some embodiments, R^{a} is methyl. In some embodiments, R³ is H, and R⁴ is a ring that is

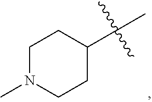 [SMILES: *C1CCN(C)CC1]

wherein the ring is substituted or unsubstituted. In some embodiments, R³ is H, and R⁴ is a ring that is

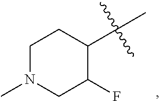 [SMILES: *C1CCN(C)CC1F]

wherein the ring is substituted or unsubstituted.

In some embodiments, the disclosure provides a compound of the formula:

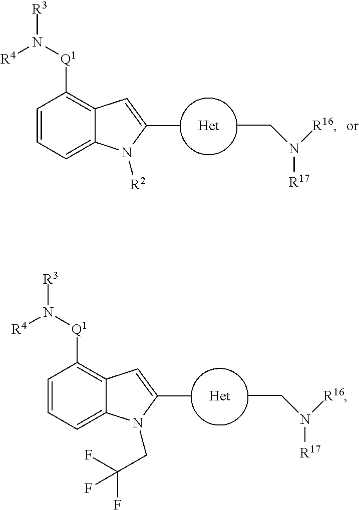

or a pharmaceutically-acceptable salt thereof, wherein the variables are as defined above.

In some embodiments, Q¹ is C═CR¹⁴R¹⁵, C═NR¹⁴, alkylene, alkenylene, or alkynylene, each of which is independently substituted or unsubstituted, C═O, C═S, or a bond. In some embodiments, Q¹ is alkylene, alkenylene, or alkynylene. In some embodiments, Q¹ is C₁-alkylene. In some embodiments, each R¹⁶ and R¹⁷ is independently alkyl, alkenyl, aryl, heteroaryl, heterocyclyl, or hydrogen. In some embodiments, Q¹ is a bond.

In some embodiments, R³ is H, and R⁴ is —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted; or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R⁴ is a ring that is:

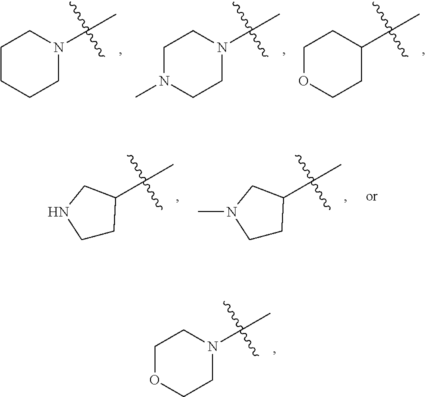

wherein the ring is substituted or unsubstituted. In some embodiments, R³ is H, and R⁴ is a ring that is

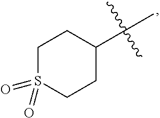 [SMILES: *C1CCS(=O)(=O)CC1]

wherein the ring is substituted or unsubstituted. In some embodiments, R³ is H, and R⁴ is a ring that is

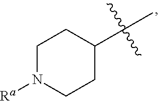

wherein the ring is substituted or unsubstituted. In some embodiments, R^{a} is alkylene. In some embodiments, R^{a} is methyl. In some embodiments, R³ is H, and R⁴ is a ring that is

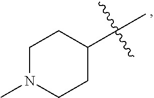 [SMILES: *C1CCN(C)CC1]

wherein the ring is substituted or unsubstituted. In some embodiments, R³ is H, and R⁴ is a ring that is

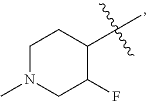

wherein the ring is substituted or unsubstituted.

In some embodiments, each R¹⁶ and R¹⁷ is independently alkyl, cycloalkyl, aryl, heteroaryl, heterocyclyl, alkoxy, carboxyl group, amino group, acyl group, acyloxy group, or an amide group, any of which is unsubstituted or substituted, or hydrogen. In some embodiments, R¹⁶ is hydrogen, and R¹⁷ is a substituted carboxyl group.

In some embodiments, the compound is of the formula:

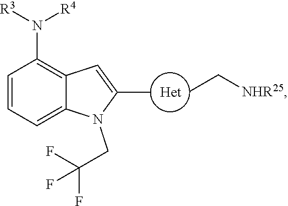

or a pharmaceutically-acceptable salt thereof, wherein R²⁵ is —C(O)R¹⁶, —C(O)NR¹⁶R¹⁷, alkyl, alkenyl, alkynyl, aryl, heteroaryl, or heterocyclyl, each of which is independently substituted or unsubstituted, or hydrogen. In some embodiments, R²⁵ is aryl that is substituted or unsubstituted. In some embodiments, R²⁵ is substituted phenyl. In some embodiments, R²⁵ is —C(O)R¹⁶, wherein R¹⁶ is alkyl, aryl, heteroaryl, or heterocyclyl. In some embodiments, R²⁵ is —C(O)R¹⁶, wherein R¹⁶ is substituted phenyl.

In some embodiments, the compound is of the formula:

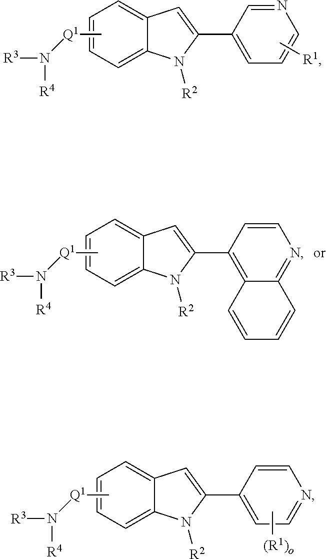

wherein:
Q¹ is C═CR¹⁴R¹⁵, C═NR¹⁴, alkylene, alkenylene, or alkynylene, each of which is independently substituted or unsubstituted, C═O, C═S, or a bond;
R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, or heterocyclyl, each of which is independently substituted or unsubstituted, or hydrogen or halogen;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
each R², R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof, wherein the variables are as defined above, and wherein o is 1, 2, 3, or 4.

In some embodiments, the compound is of the formula:

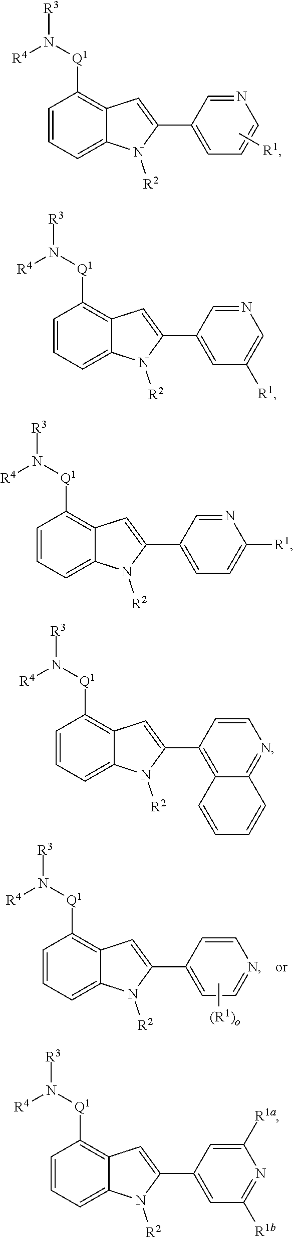

wherein:
Q¹ is C═CR¹⁴R¹⁵, C═NR¹⁴, alkylene, alkenylene, or alkynylene, each of which is independently substituted or unsubstituted, or C═O, C═S, or a bond;
each R¹, R^{1a}, and R^{1b} is independently —C(O)R¹⁶, —C(O)OR¹⁶, —C(O)NR¹⁶R¹⁷, —OR¹⁶, —SR¹⁶, —NR¹⁶R¹⁷, —NR¹C(O)R¹⁶, —OC(O)R¹⁶, —SiR¹⁶R¹⁷R¹⁸, alkyl, alkenyl, alkynyl, alkoxy, aryl, heteroaryl, or heterocyclyl, each of which is independently substituted or unsubstituted, or hydrogen or halogen;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
o is 0, 1, 2, 3, or 4;
each R², R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, each R^{1a} and R^{1b} is independently alkyl, alkoxy, aryl, heteroaryl, heterocyclyl, or NR¹⁶R¹⁷. In some embodiments, R^{1a} is unsubstituted phenyl, and R^{1b} is amino.

In some embodiments, the compound is of the formula:

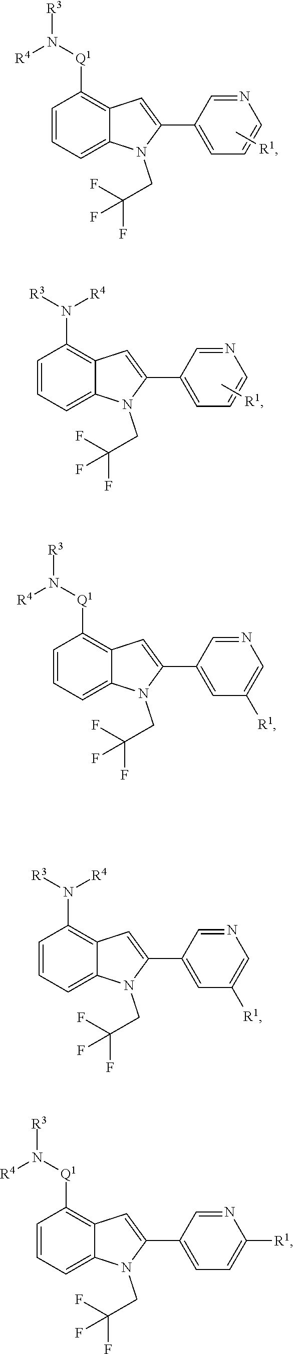

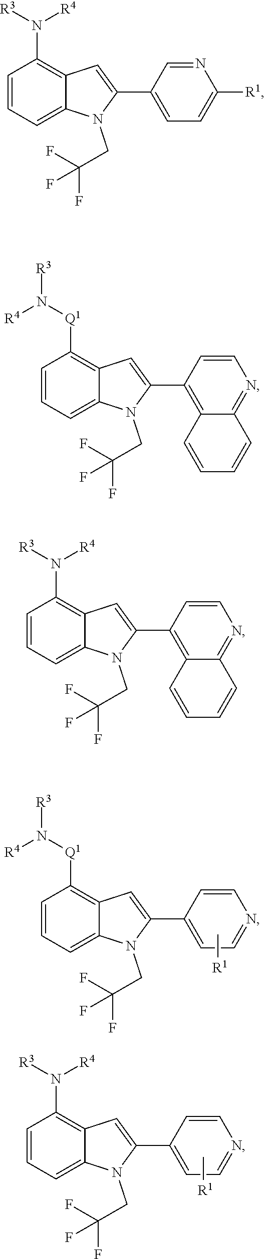

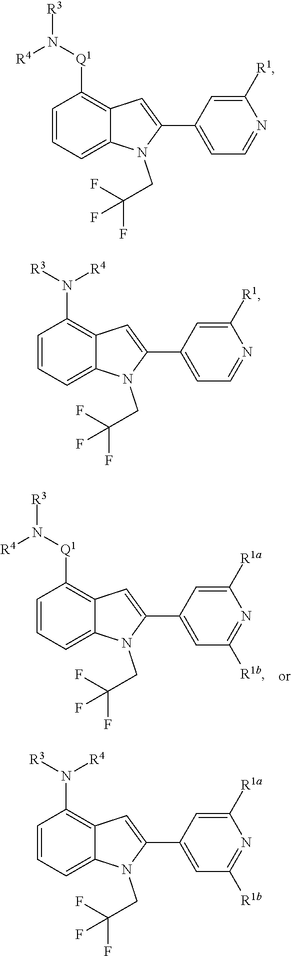

or a pharmaceutically-acceptable salt thereof, wherein the variables are as defined above.

In some embodiments, R¹ is —C(O)NR¹⁶R¹⁷, alkyl, alkenyl, alkynyl, alkoxy, aryl, heteroaryl, or heterocyclyl, each of which is independently substituted or unsubstituted, or hydrogen or halogen. In some embodiments, R¹ is alkyl, alkoxy, aryl, or halo. In some embodiments, R¹ is methoxy, methyl, or phenyl. In some embodiments, each R^{1a} and R^{1b} is independently alkyl, alkoxy, aryl, heteroaryl, heterocyclyl, or NR¹⁶R¹⁷. In some embodiments, R^{1a} is unsubstituted phenyl, and R^{1b} is amino.

In some embodiments, Q¹ is C═CR¹⁴R¹⁵, C═NR¹⁴, alkylene, alkenylene, or alkynylene, each of which is independently substituted or unsubstituted, or C═O, C═S, or a bond. In some embodiments, Q¹ is alkylene, alkenylene, or alkynylene. In some embodiments, Q¹ is C₁-alkylene. In some embodiments, each R¹⁶ and R¹⁷ is independently alkyl, alkenyl, aryl, heteroaryl, heterocyclyl, or hydrogen. In some embodiments, Q¹ is a bond.

In some embodiments, R³ is H, and R⁴ is —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted; or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R⁴ is a ring that is:

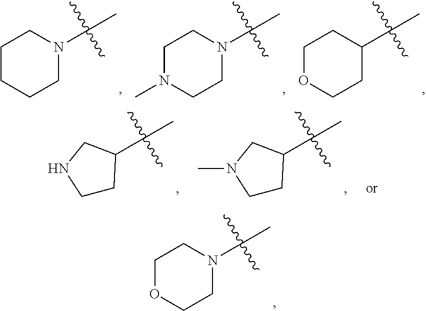

wherein the ring is substituted or unsubstituted. In some embodiments, R³ is H, and R⁴ is a ring that is

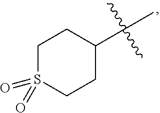 [SMILES: *C1CCS(=O)(=O)CC1]

wherein the ring is substituted or unsubstituted. In some embodiments, R³ is H, and R⁴ is a ring that is

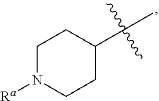

wherein the ring is substituted or unsubstituted. In some embodiments, R^{a} is alkylene. In some embodiments, R^{a} is methyl. In some embodiments, R³ is H, and R⁴ is a ring that is

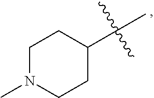 [SMILES: *C1CCN(C)CC1]

wherein the ring is substituted or unsubstituted. In some embodiments, R³ is H, and R⁴ is a ring that is

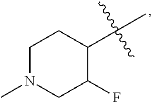 [SMILES: *C1CCN(C)CC1F]

wherein the ring is substituted or unsubstituted.

In some embodiments, each R¹⁶ and R¹⁷ is independently alkyl, cycloalkyl, aryl, heteroaryl, heterocyclyl, alkoxy, carboxyl group, amino group, acyl group, acyloxy group, or an amide group, any of which is unsubstituted or substituted, or hydrogen. In some embodiments, R¹⁶ is hydrogen, and R¹⁷ is a substituted carboxyl group.

In some embodiments, the compound is of the formula:

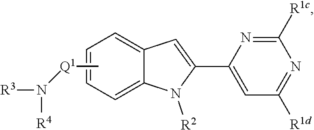

wherein:
Q¹ is C═CR¹⁴R¹⁵, C═NR¹⁴, alkylene, alkenylene, or alkynylene, each of which is independently substituted or unsubstituted, or C═O, C═S, or a bond;
each R^{1c} and R^{1d} is independently —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, or heterocyclyl, each of which is independently substituted or unsubstituted, or hydrogen or halogen;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
each R², R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, each R^{1c} and R^{1d} is independently —OR¹⁶, —NR¹⁶R¹⁷, —NR¹⁶C(O)R¹⁶, alkyl, alkenyl, alkynyl, alkoxy, aryl, heteroaryl, or heterocyclyl, each of which is independently substituted or unsubstituted, or hydrogen.

In some embodiments, the compound is of the formula:

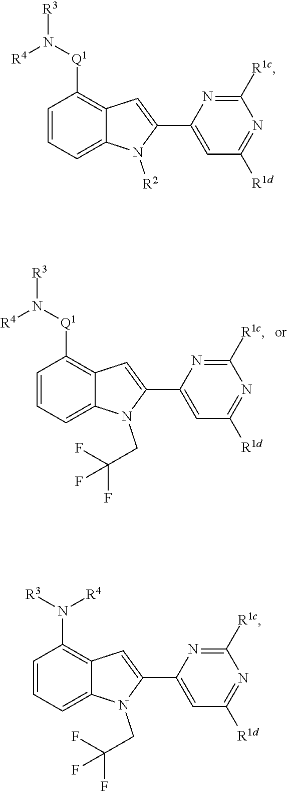

or a pharmaceutically-acceptable salt thereof, wherein the variables are as defined above.

In some embodiments, each R^{1c} and R^{1d} is independently —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, or heterocyclyl, each of which is independently substituted or unsubstituted, or hydrogen or halogen. In some embodiments, R^{1c} is amino, and R^{1d} is phenyl. In some embodiments, R^{1c} is amino, and R^{1d} is cycloalkenyl.

In some embodiments, the compound is of the formula:

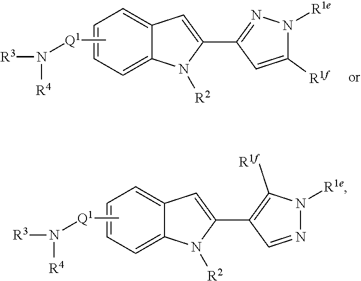

wherein:
Q¹ is C═CR¹⁴R¹⁵, C═NR¹⁴, alkylene, alkenylene, or alkynylene, each of which is independently substituted or unsubstituted, or C═O, C═S, or a bond;
each R^{1e} and R^{1f} is independently —C(O)R¹⁶, —C(O)OR¹⁶, —C(O)NR¹⁶R¹⁷, —OR¹⁶, —SR¹⁶, —NR¹⁶R¹⁷—NR¹⁶C(O)R¹⁶, —OC(O)R¹⁶, —SiR¹⁶R¹⁷R¹⁸, alkyl, alkenyl, alkynyl, alkoxy, aryl, heteroaryl, or heterocyclyl, each of which is independently substituted or unsubstituted, or hydrogen or halogen;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
each R², R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, the compound is of the formula:

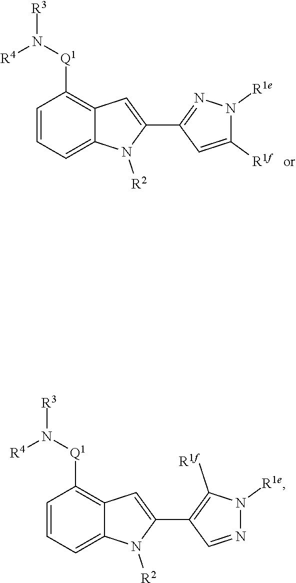

or a pharmaceutically-acceptable salt thereof, wherein the variables are as defined above.

In some embodiments, the compound is of the formula:

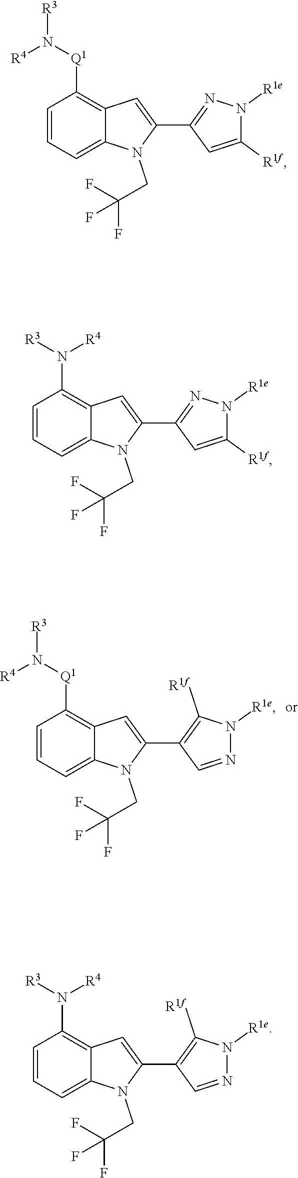

or a pharmaceutically-acceptable salt thereof, wherein the variables are as defined above.

In some embodiments, Q¹ is C═CR¹⁴R¹⁵, C═NR¹⁴, alkylene, alkenylene, or alkynylene, each of which is independently substituted or unsubstituted, C═O, C═S, or a bond. In some embodiments, Q¹ is alkylene, alkenylene, or alkynylene. In some embodiments, Q¹ is C₁-alkylene. In some embodiments, each R¹⁶ and R¹⁷ is independently alkyl, alkenyl, aryl, heteroaryl, heterocyclyl, or hydrogen. In some embodiments, Q¹ is a bond.

In some embodiments, R³ is H, and R⁴ is —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted; or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R⁴ is a ring that is:

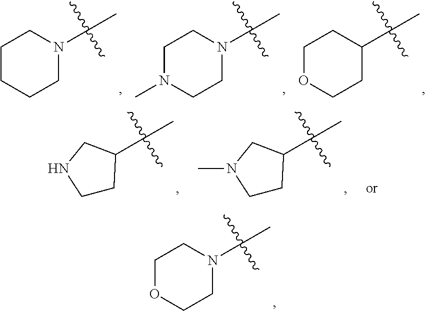

wherein the ring is substituted or unsubstituted. In some embodiments, R³ is H, and R⁴ is a ring that is

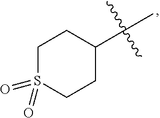 [SMILES: *C1CCS(=O)(=O)CC1]

wherein the ring is substituted or unsubstituted. In some embodiments, R³ is H, and R⁴ is a ring that is

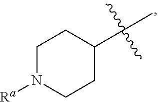

wherein the ring is substituted or unsubstituted. In some embodiments, R^{a} is alkylene. In some embodiments, R^{a} is methyl. In some embodiments, R³ is H, and R⁴ is a ring that is

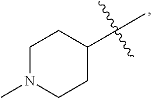 [SMILES: *C1CCN(C)CC1]

wherein the ring is substituted or unsubstituted. In some embodiments, R³ is H, and R⁴ is a ring that is

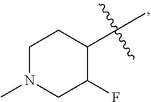 [SMILES: *C1CCN(C)CC1F]

wherein the ring is substituted or unsubstituted.

In some embodiments, each R^{1e} and R^{1f} is independently alkyl, NR¹⁶R¹⁷, aryl, heteroaryl, or heterocyclyl, each of which is independently substituted or unsubstituted, or hydrogen. In some embodiments, R^{1e} is substituted alkyl, and R^{1f} is hydrogen. In some embodiments, R^{1e} is hydrogen, and R^{1f} is NR¹⁶R¹⁷, wherein each R¹⁶ and R¹⁷ is independently alkyl, alkenyl, aryl, heteroaryl, or heterocyclyl, each of which is independently substituted or unsubstituted, or hydrogen. In some embodiments, R^{1e} is hydrogen, and R^{1f} is NR¹⁶R¹⁷, wherein R¹⁶ is hydrogen, and R¹⁷ is alkyl. In some embodiments, R^{1e} is hydrogen, and R^{1f} is NR¹⁶R¹⁷, wherein R¹⁶ is hydrogen, and R¹⁷ is phenyl. In some embodiments, R^{1e} is hydrogen, and R^{1f} is amino.

In some embodiments, the compound is of the formula:

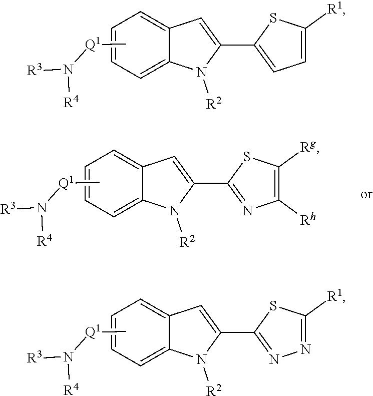

wherein:
Q¹ is C═CR¹⁴R¹⁵, C═NR¹⁴, alkylene, alkenylene, or alkynylene, each of which is independently substituted or unsubstituted, C═O, C═S, or a bond;
each R¹, R^{1g}, and R^{1h} is independently —C(O)R¹⁶, —C(O)OR¹⁶, —C(O)NR¹⁶R¹⁷, —OR¹⁶, —SR¹⁶, —NR¹⁶R¹⁷, —NR¹C(O)R¹⁶, —OC(O)R¹⁶, —SiR¹⁶R¹⁷R¹⁸, alkyl, alkenyl, alkynyl, alkoxy, aryl, heteroaryl, or heterocyclyl, each of which is independently substituted or unsubstituted, or hydrogen or halogen;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
each R², R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, the compound is of the formula:

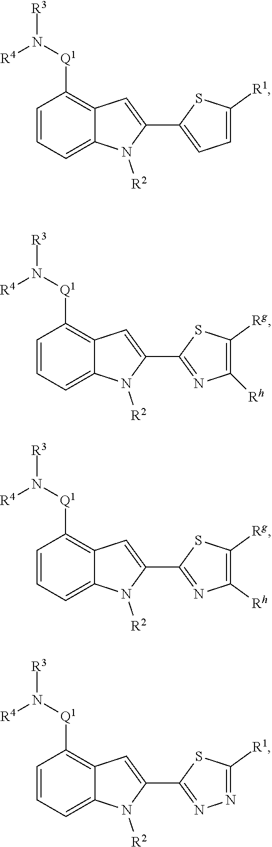

or a pharmaceutically-acceptable salt thereof, wherein the variables are as defined above.

In some embodiments, the compound is of the formula:

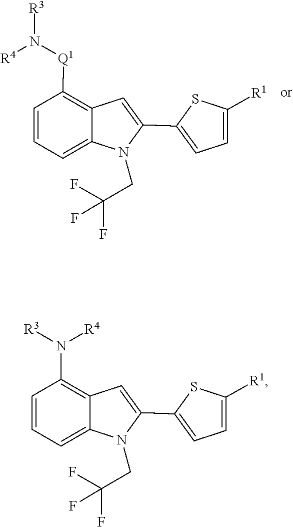

or a pharmaceutically-acceptable salt thereof, wherein the variables are as defined above.

In some embodiments, Q¹ is C═CR¹⁴R¹⁵, C═NR¹⁴, alkylene, alkenylene, or alkynylene, each of which is independently substituted or unsubstituted, C═O, C═S, or a bond. In some embodiments, Q¹ is alkylene, alkenylene, or alkynylene. In some embodiments, Q¹ is C₁-alkylene. In some embodiments, each R¹⁶ and R¹⁷ is independently alkyl, alkenyl, aryl, heteroaryl, heterocyclyl, or hydrogen. In some embodiments, Q¹ is a bond.

In some embodiments, R³ is H, and R⁴ is —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted; or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R⁴ is a ring that is:

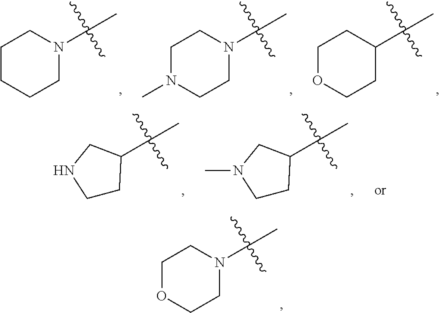

wherein the ring is substituted or unsubstituted. In some embodiments, R³ is H, and R⁴ is a ring that is

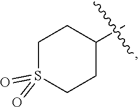

wherein the ring is substituted or unsubstituted. In some embodiments, R³ is H, and R⁴ is a ring that is

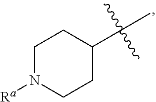

wherein the ring is substituted or unsubstituted. In some embodiments, R^{a} is alkylene. In some embodiments, R^{a} is methyl. In some embodiments, R³ is H, and R⁴ is a ring that is

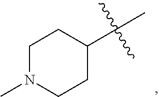 [SMILES: *C1CCN(C)CC1]

wherein the ring is substituted or unsubstituted. In some embodiments, R³ is H, and R⁴ is a ring that is

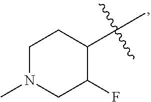 [SMILES: *C1CCN(C)CC1F]

wherein the ring is substituted or unsubstituted.

In some embodiments, R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, or heterocyclyl, each of which is independently substituted or unsubstituted, or hydrogen or halogen. In some embodiments, R¹ is substituted alkyl. In some embodiments, R¹ is alkyl substituted with NR¹⁶R¹⁷, wherein each R¹⁶ and R¹⁷ is independently alkyl, cycloalkyl, aryl, heteroaryl, heterocyclyl, alkoxy, carboxyl group, amino group, acyl group, acyloxy group, or an amide group, any of which is unsubstituted or substituted, or hydrogen. In some embodiments, R¹⁶ is hydrogen, and R¹⁷ is a substituted carboxyl group. In some embodiments, R¹⁶ is hydrogen, and R¹⁷ is carboxyl substituted with alkyl or aryl. In some embodiments, R¹⁶ is hydrogen, and R¹⁷ is carboxyl substituted with cycloalkyl or phenyl. In some embodiments, R¹⁶ and R¹⁷ are hydrogen.

In some embodiments, the compound is of the formula:

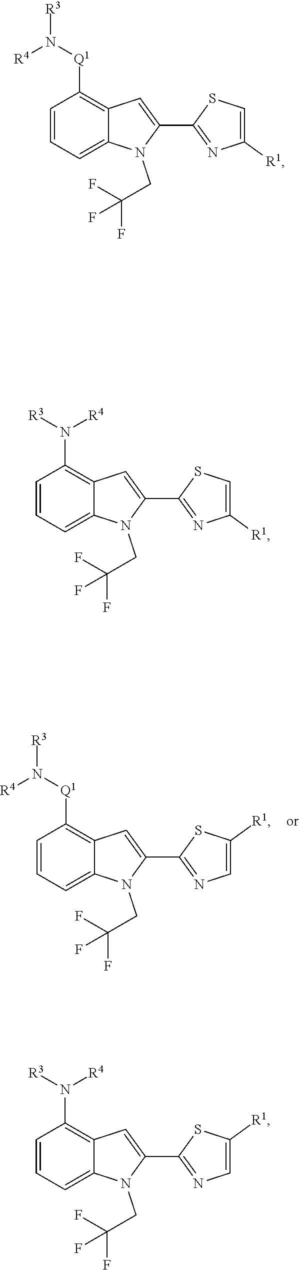

or a pharmaceutically-acceptable salt thereof, wherein the variables are as defined above.

In some embodiments, R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, or heterocyclyl, each of which is independently substituted or unsubstituted, or hydrogen or halogen. In some embodiments, R¹ is substituted alkyl. In some embodiments, R¹ is alkyl substituted with NR¹⁶R¹⁷, wherein each R¹⁶ and R¹⁷ is independently alkyl, cycloalkyl, aryl, heteroaryl, heterocyclyl, alkoxy, carboxyl group, amino group, acyl group, acyloxy group, or an amide group, any of which is unsubstituted or substituted, or hydrogen. In some embodiments, R¹⁶ is hydrogen, and R¹⁷ is a substituted carboxyl group. In some embodiments, R¹⁶ is hydrogen, and R¹⁷ is carboxyl substituted with alkyl or aryl. In some embodiments, R¹⁶ is hydrogen, and R¹⁷ is carboxyl substituted with cycloalkyl or phenyl. In some embodiments, R¹⁶ and R¹⁷ are hydrogen.

In some embodiments, the compound is of the formula:

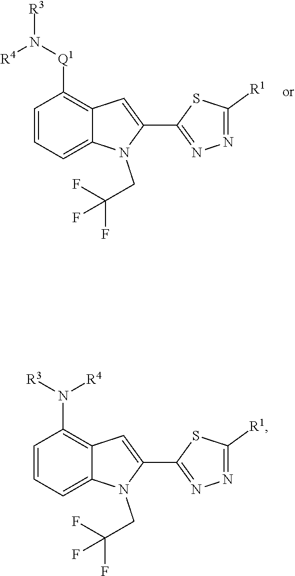

or a pharmaceutically-acceptable salt thereof, wherein the variables are as defined above.

In some embodiments, Q¹ is C═CR¹⁴R¹⁵, C═NR¹⁴, alkylene, alkenylene, or alkynylene, each of which is independently substituted or unsubstituted, C═O, C═S, or a bond. In some embodiments, Q¹ is alkylene, alkenylene, or alkynylene. In some embodiments, Q¹ is C₁-alkylene. In some embodiments, each R¹⁶ and R¹⁷ is independently alkyl, alkenyl, aryl, heteroaryl, heterocyclyl, or hydrogen. In some embodiments, Q¹ is a bond.

In some embodiments, R³ is H, and R⁴ is —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted; or hydrogen. In some embodiments, R³ is H, and R⁴ is alkyl, alkenyl, alkynyl, aryl, heteroaryl, or heterocyclyl, each of which is independently substituted or unsubstituted, or hydrogen. In some embodiments, R⁴ is heterocyclyl. In some embodiments, R⁴ is piperidinyl, piperazinyl, tetahydropyranyl, morpholinyl, or pyrrolidinyl, each of which is independently substituted or unsubstituted.

In some embodiments, R⁴ is a ring that is:

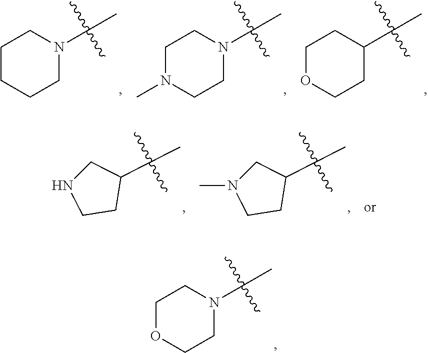

wherein the ring is substituted or unsubstituted. In some embodiments, R³ is H, and R⁴ is a ring that is

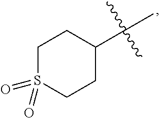 [SMILES: *C1CCS(=O)(=O)CC1]

wherein the ring is substituted or unsubstituted. In some embodiments, R³ is H, and R⁴ is a ring that is

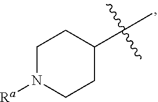

wherein the ring is substituted or unsubstituted. In some embodiments, R^{a} is alkylene. In some embodiments, R^{a} is methyl. In some embodiments, R³ is H, and R⁴ is a ring that is

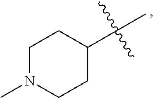 [SMILES: *C1CCN(C)CC1]

wherein the ring is substituted or unsubstituted. In some embodiments, R³ is H, and R⁴ is a ring that is

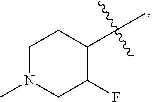 [SMILES: *C1CCN(C)CC1F]

wherein the ring is substituted or unsubstituted.

In some embodiments, R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, or heterocyclyl, each of which is independently substituted or unsubstituted, or hydrogen or halogen. In some embodiments R¹ is substituted alkyl. In some embodiments, R¹ is alkyl substituted with NR¹⁶R¹⁷, wherein each R¹⁶ and R¹⁷ is independently alkyl, cycloalkyl, aryl, heteroaryl, heterocyclyl, alkoxy, carboxyl group, amino group, acyl group, acyloxy group, or an amide group, any of which is unsubstituted or substituted, or hydrogen. In some embodiments, R¹⁶ is hydrogen, and R¹⁷ is aryl, heteroaryl, carboxyl, or hydrogen. In some embodiments, R¹⁶ is hydrogen, and R¹⁷ is carboxyl substituted with aryl, heteroaryl, cycloalkyl, or alkyl. In some embodiments, R¹⁶ and R¹⁷ are hydrogen.

In some embodiments, the compound is of the formula:

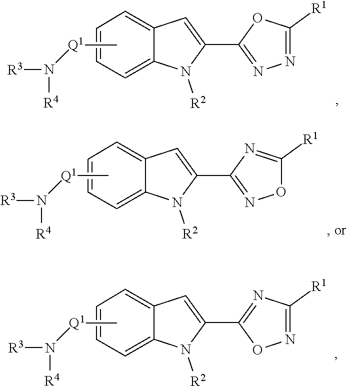

wherein:
Q¹ is C═CR¹⁴R¹⁵, C═NR¹⁴, alkylene, alkenylene, or alkynylene, each of which is independently substituted or unsubstituted, C═O, C═S, or a bond;
R¹ is —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, or heterocyclyl, each of which is independently substituted or unsubstituted, or hydrogen or halogen;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
each R², R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, the compound is of the formula:

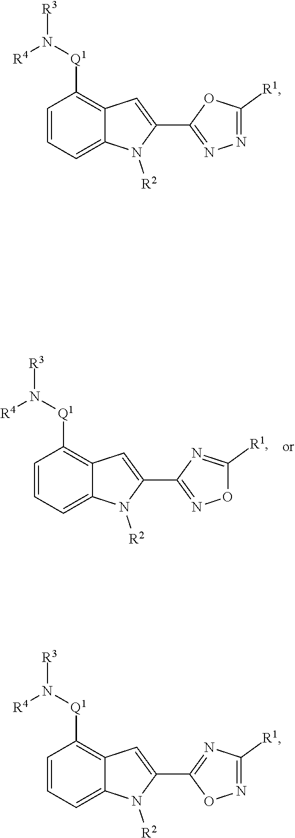

or a pharmaceutically-acceptable salt thereof, wherein the variables are as defined above.

In some embodiments, the compound is of the formula:

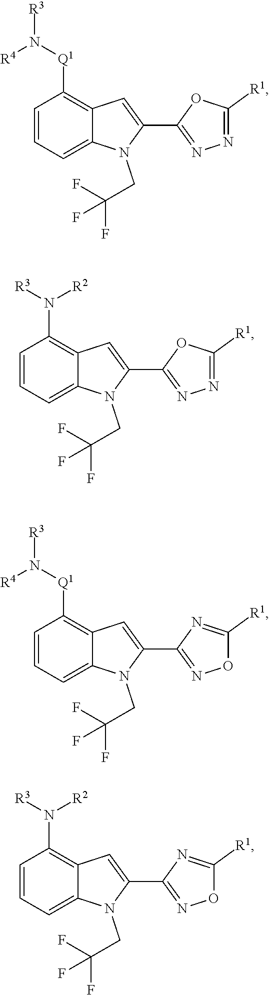

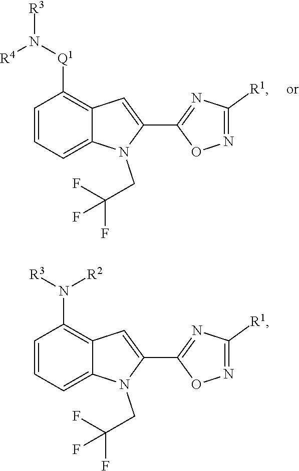

or a pharmaceutically-acceptable salt thereof, wherein the variables are as defined above.

In some embodiments, the compound is of the formula:

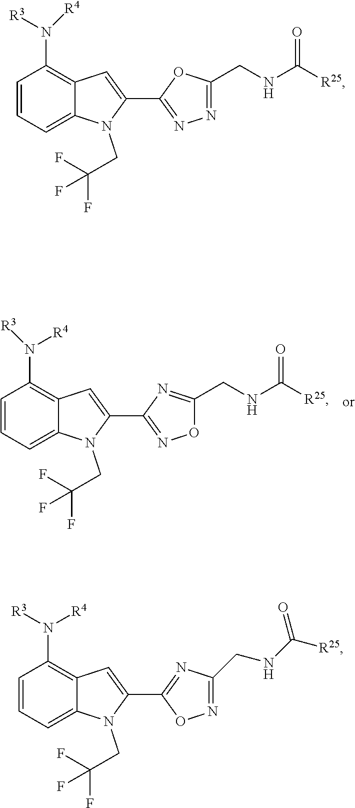

wherein:
Q¹ is C═CR¹⁴R¹⁵, C═NR¹⁴, alkylene, alkenylene, or alkynylene, each of which is independently substituted or unsubstituted, C═O, C═S, or a bond;
each R^{1c} and R^{1d} is independently —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, alkyl, alkenyl, alkynyl, alkoxy, aryl, heteroaryl, or heterocyclyl, each of which is independently substituted or unsubstituted, or hydrogen or halogen;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
each R¹⁴, R¹⁵, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
R²⁵ is alkyl, alkenyl, alkynyl, aryl, heteroaryl, or heterocyclyl, each of which is independently substituted or unsubstituted, or hydrogen;
   or a pharmaceutically-acceptable salt thereof.

In some embodiments, R²⁵ is heterocyclyl, cycloalkyl, aryl, each of which is substituted or unsubstituted. In some embodiments, R²⁵ is phenyl or cyclopropyl, each of which is substituted or unsubstituted. In some embodiments, R²⁵ is substituted cyclopropyl. In some embodiments, R²⁵ is heteroaryl or heterocyclyl, each of which is substituted or unsubstituted. In some embodiments, R²⁵ is thiophenyl, indolenyl, or pyrrolyl, each of which is substituted or unsubstituted.

Non-limiting examples of compounds of the disclosure include compounds of any of the following formulae:

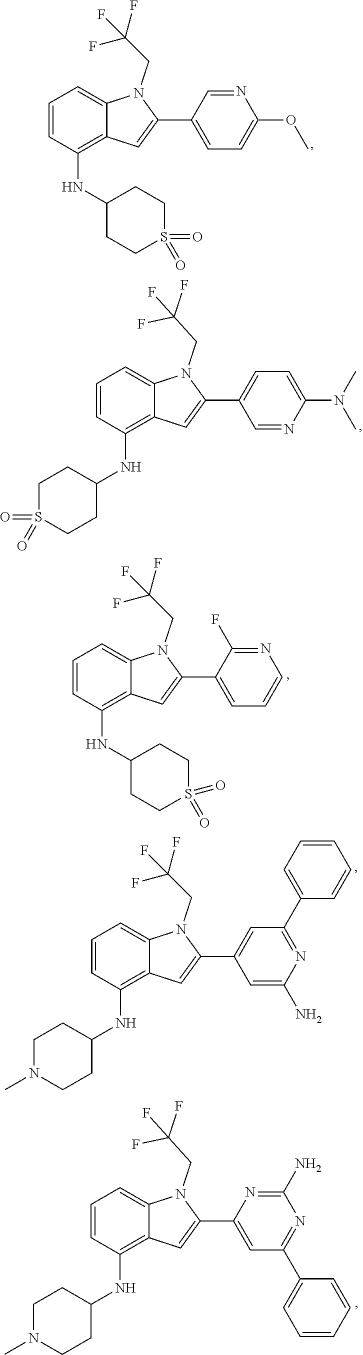

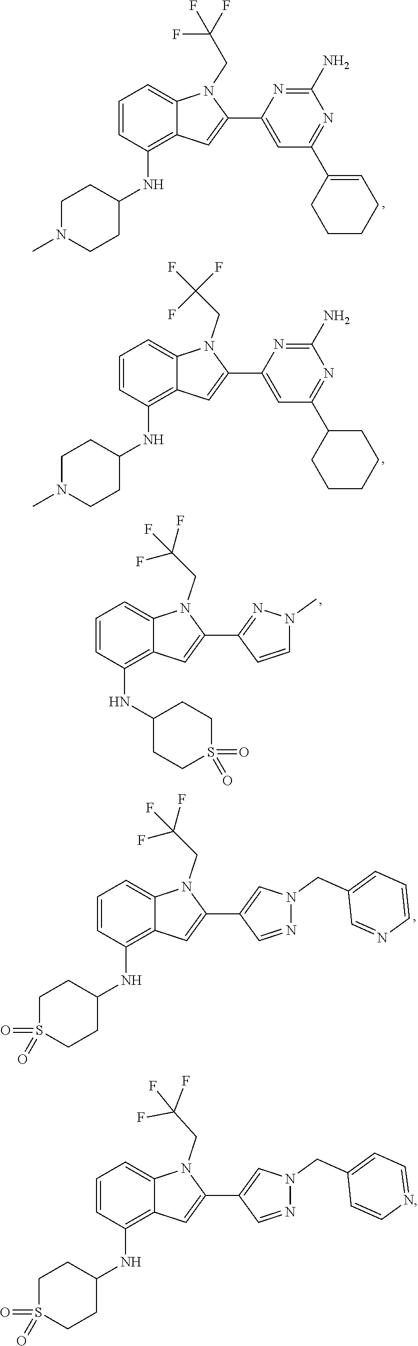

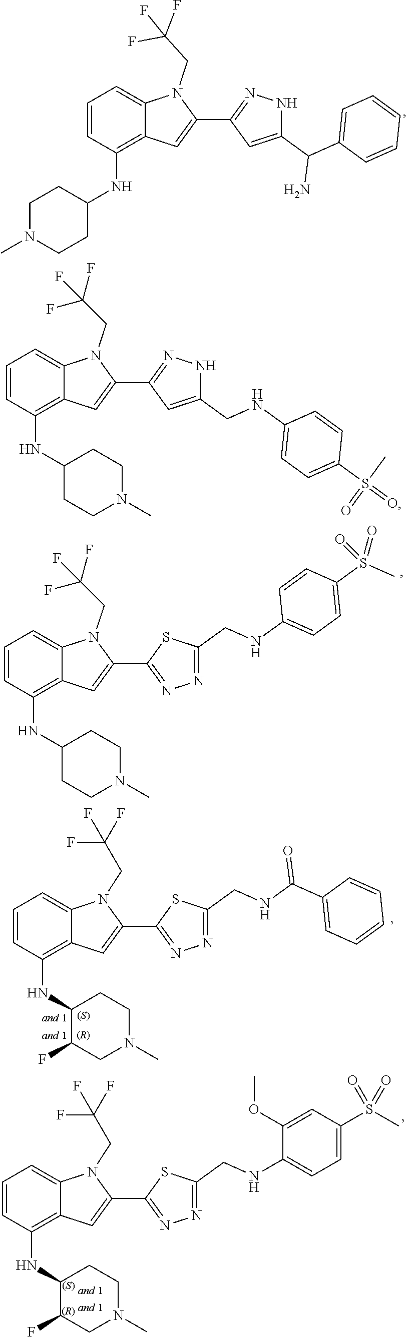

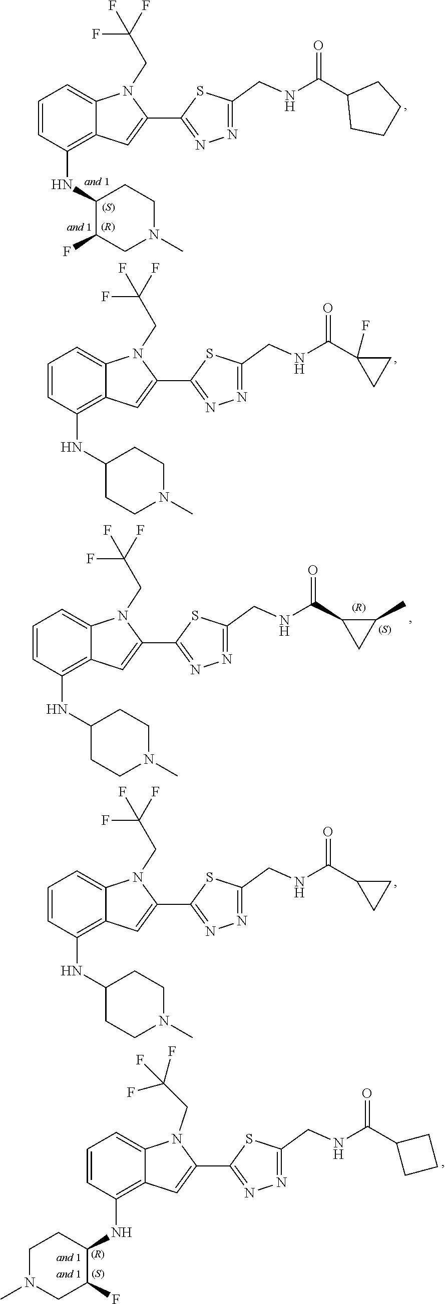

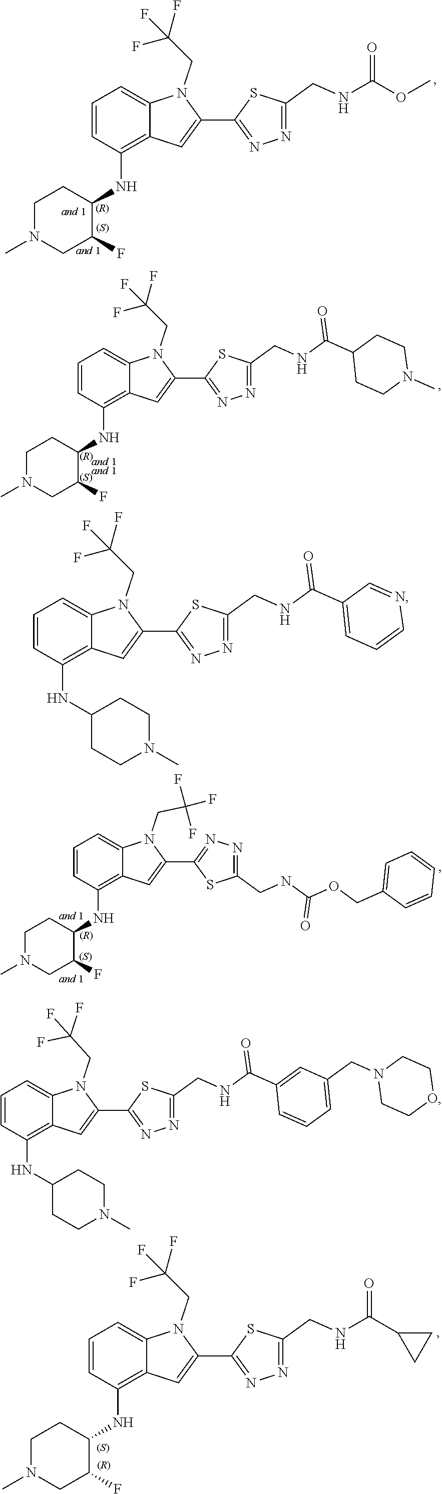

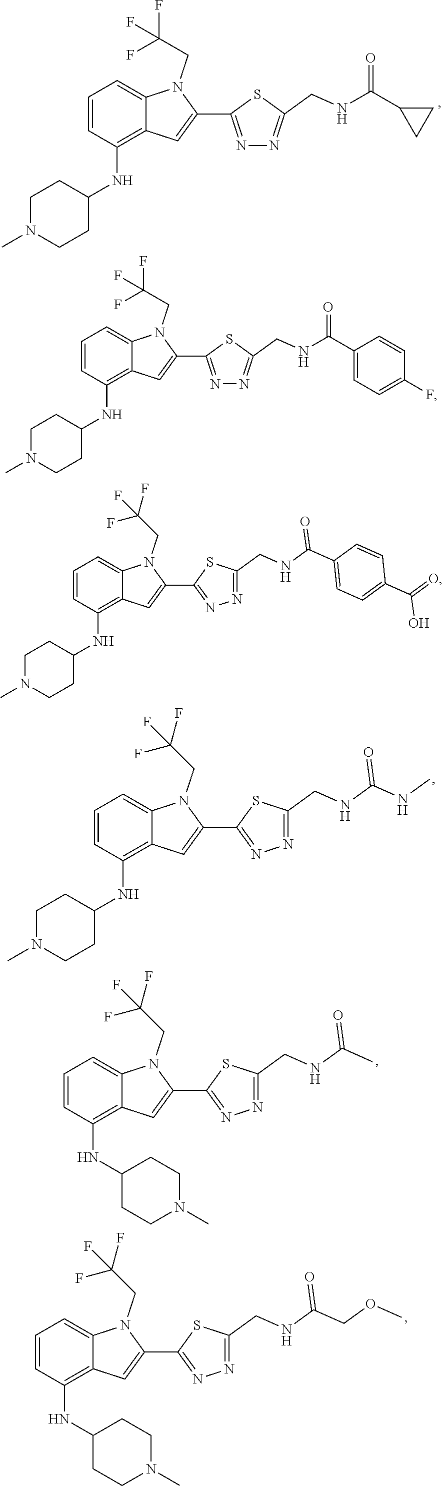

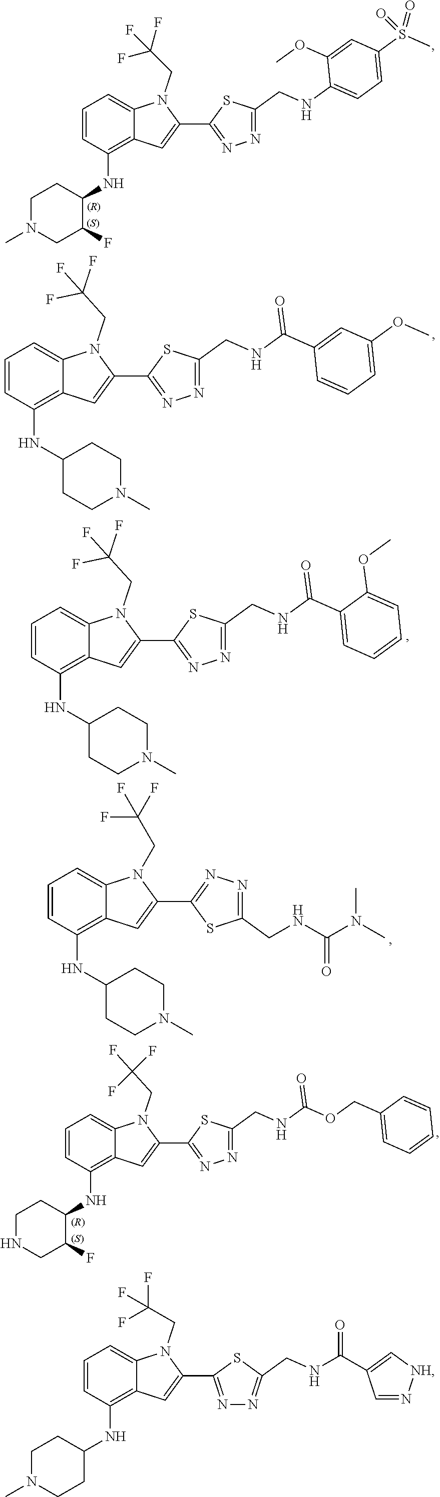

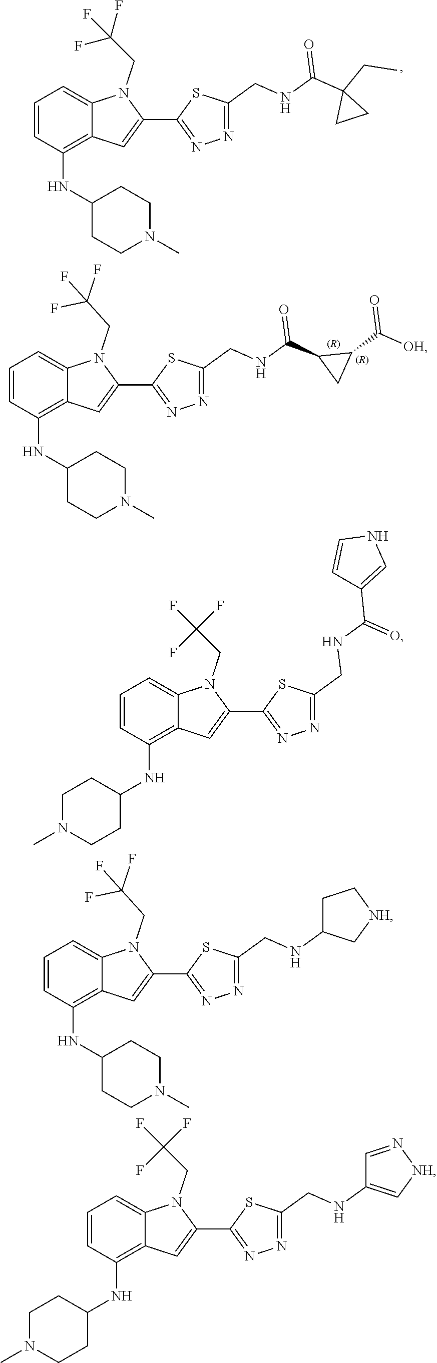

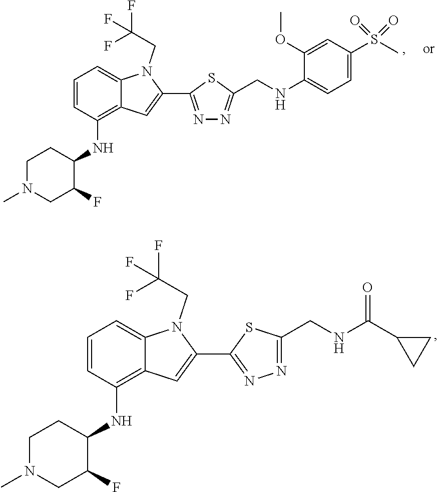

or a pharmaceutically-acceptable salt thereof.

Non-limiting examples of compounds of the disclosure include compounds of any of the following formulae:

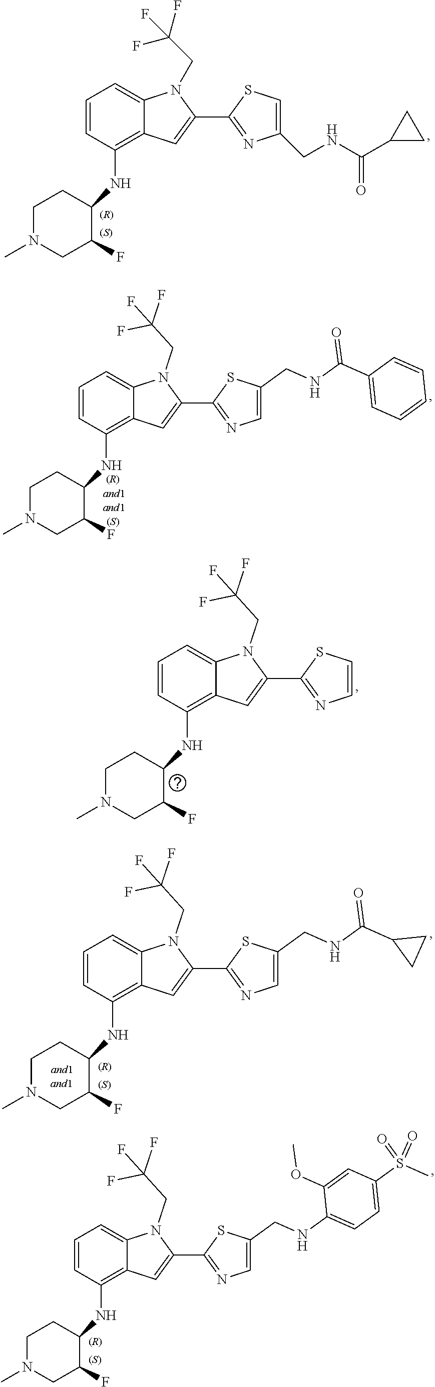

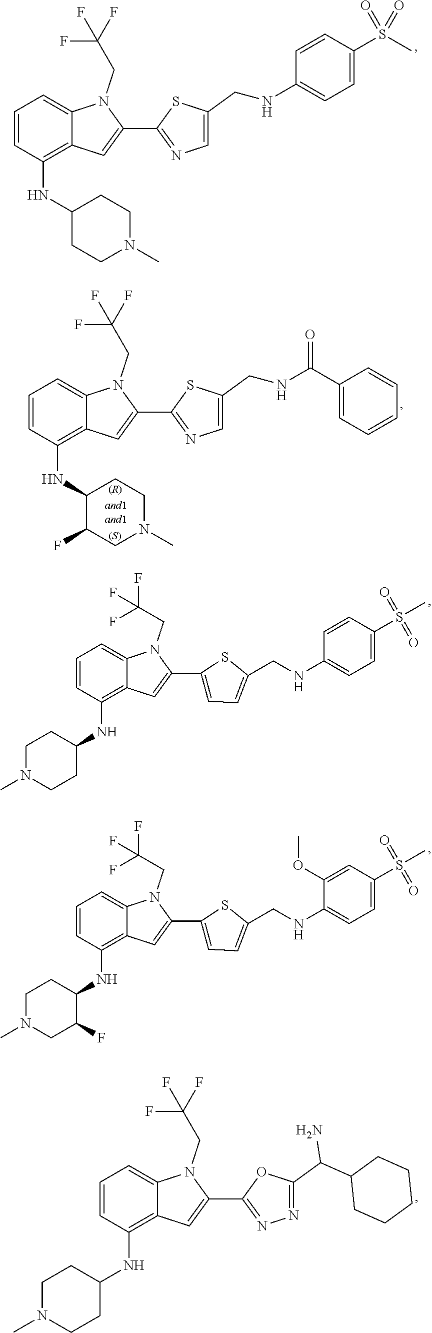

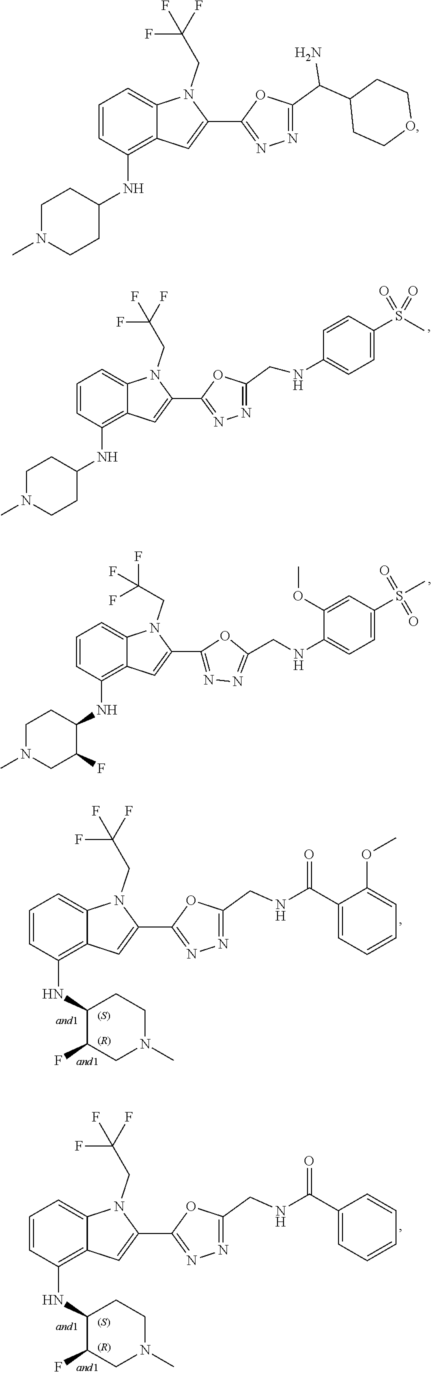

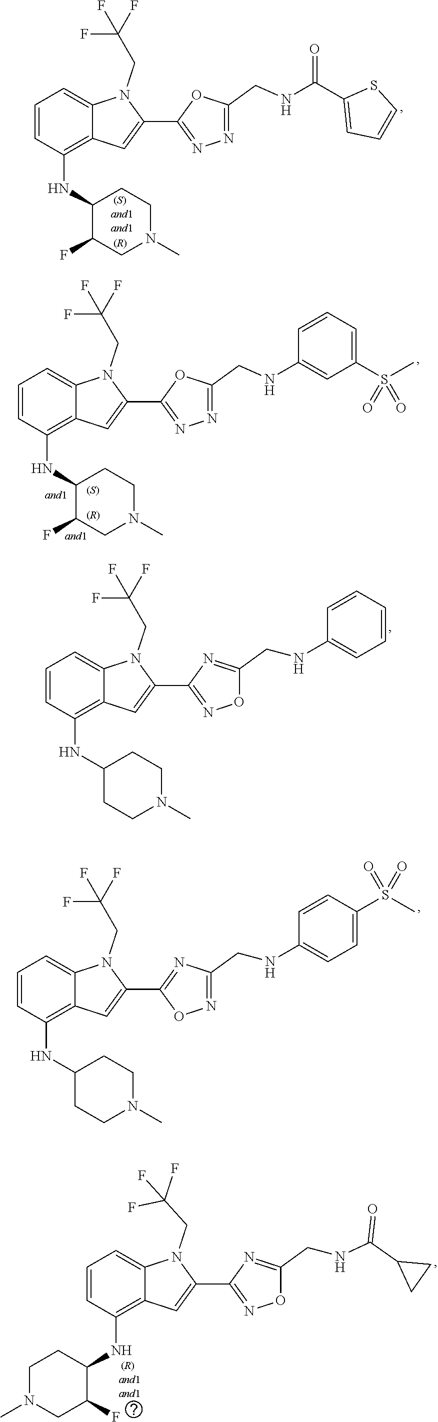

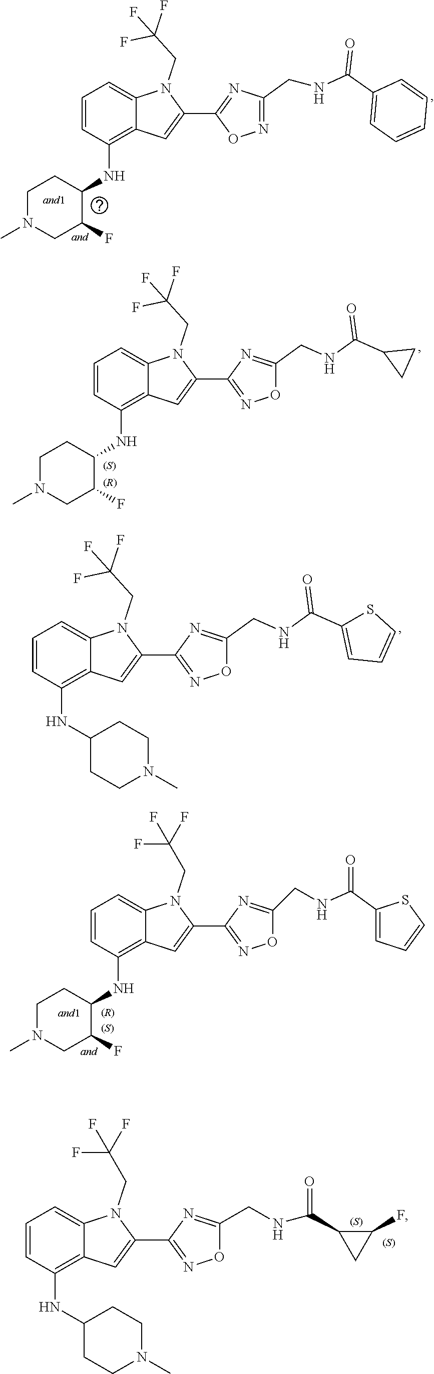

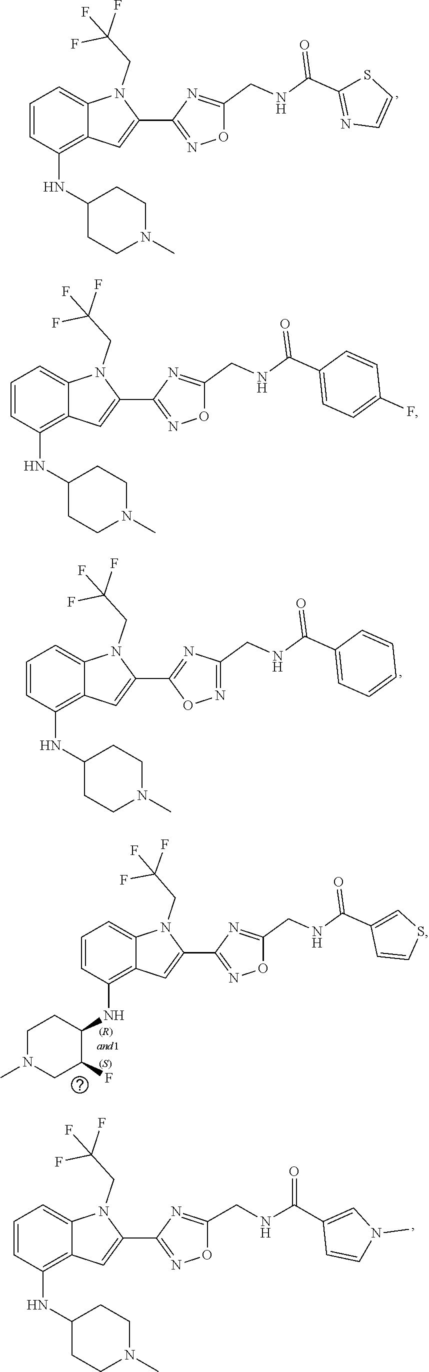

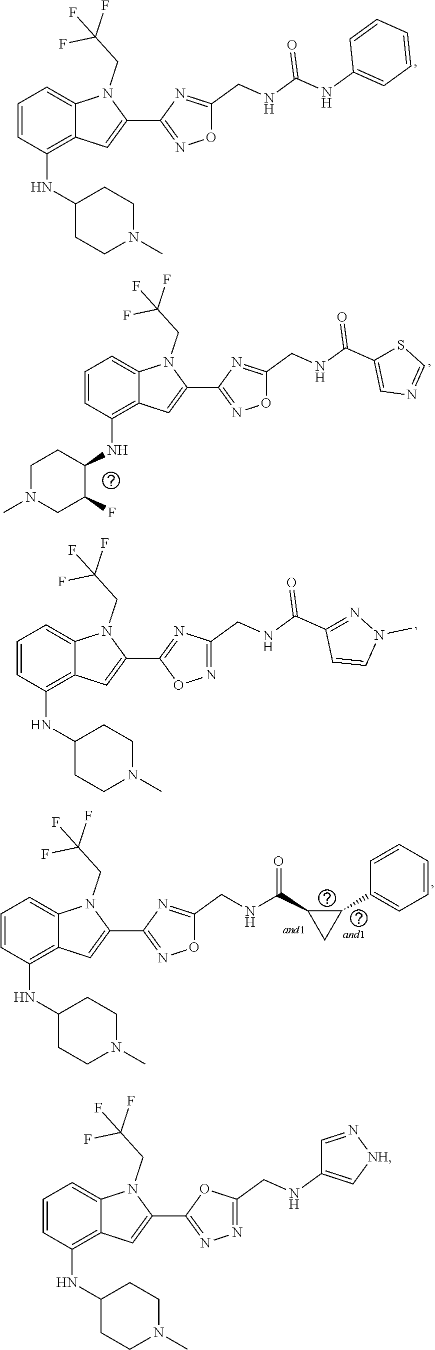

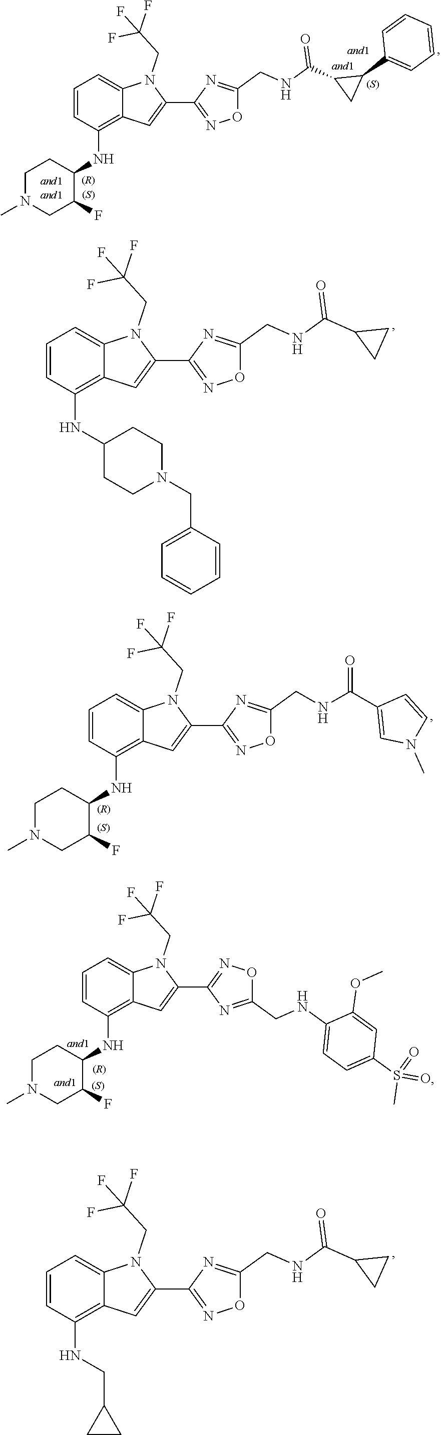

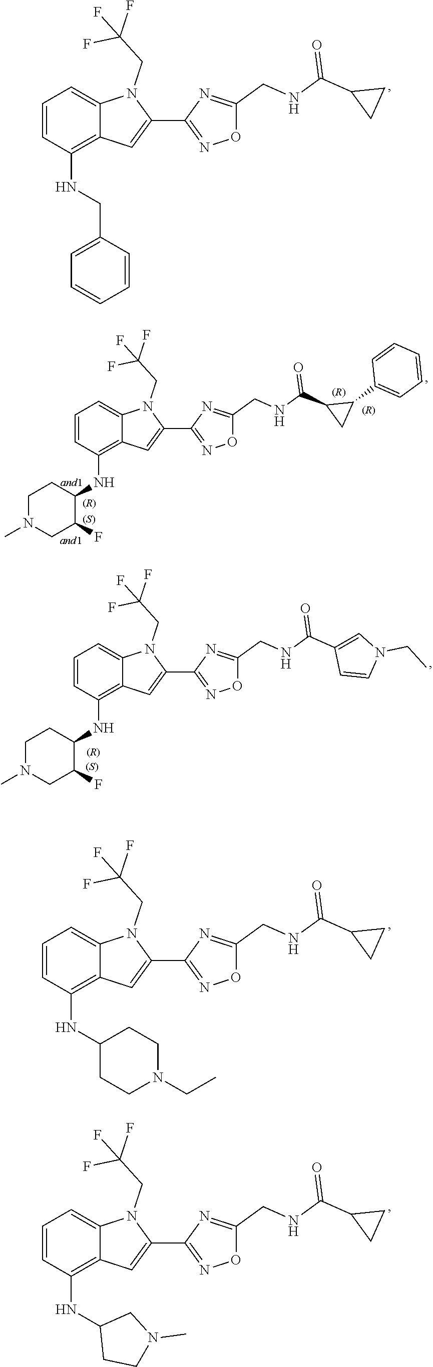

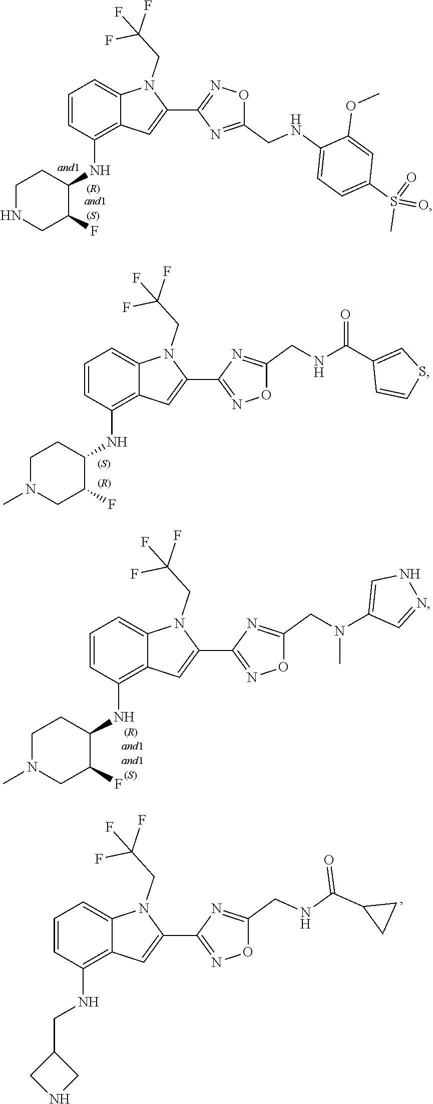

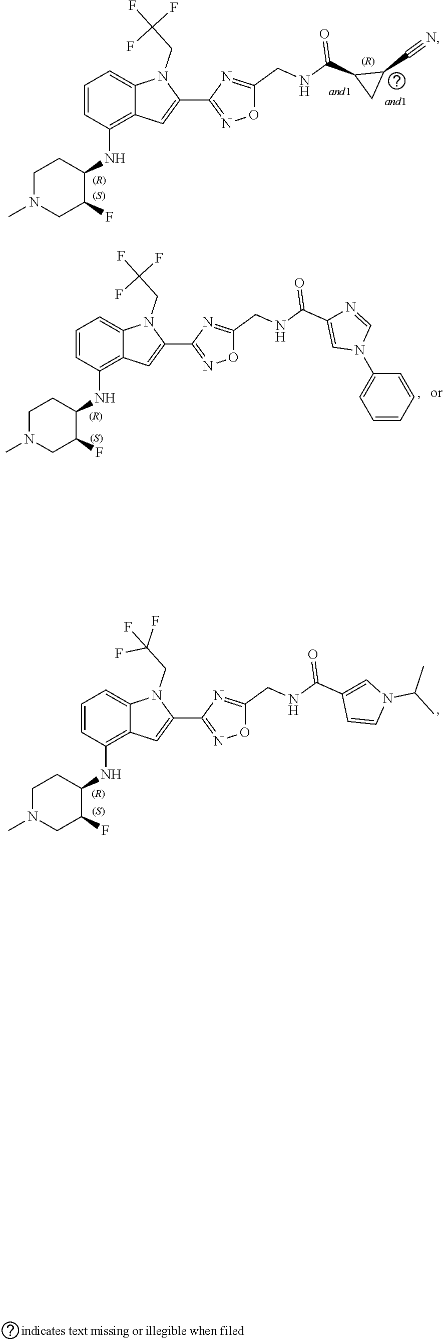

or a pharmaceutically-acceptable salt thereof.

Compounds herein can include all stereoisomers, enantiomers, diastereomers, mixtures, racemates, atropisomers, and tautomers thereof.

Compounds herein can comprise one or more isotopically enriched atoms. For example, compounds having a hydrogen atom is replaced by a deuterium or deuterio group [D], are within the scope of this disclosure. In some embodiments, compounds herein can be substituted with at least one deuterium.

Non-limiting examples of optional substituents include hydroxyl groups, sulfhydryl groups, halogens, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, ureido groups, epoxy groups, and ester groups.

Non-limiting examples of alkyl and alkylene groups include straight, branched, and cyclic alkyl and alkylene groups. An alkyl or alkylene group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

Non-limiting examples of straight alkyl groups include methyl, ethyl, propyl, butyl, pentyl, hexyl, heptyl, octyl, nonyl, and decyl.

Branched alkyl groups include any straight alkyl group substituted with any number of alkyl groups. Non-limiting examples of branched alkyl groups include isopropyl, isobutyl, sec-butyl, and t-butyl.

Non-limiting examples of substituted alkyl groups includes hydroxymethyl, chloromethyl, trifluoromethyl, aminomethyl, 1-chloroethyl, 2-hydroxyethyl, 1,2-difluoroethyl, and 3-carboxypropyl.

Non-limiting examples of cyclic alkyl groups include cyclopropyl, cyclobutyl, cyclopentyl, cyclohexyl, cycloheptlyl, and cyclooctyl groups. Cyclic alkyl groups also include fused-, bridged-, and spiro-bicycles and higher fused-, bridged-, and spiro-systems. A cyclic alkyl group can be substituted with any number of straight, branched, or cyclic alkyl groups. Non-limiting examples of cyclic alkyl groups include cyclopropyl, 2-methyl-cycloprop-1-yl, cycloprop-2-en-1-yl, cyclobutyl, 2,3-dihydroxycyclobut-1-yl, cyclobut-2-en-1-yl, cyclopentyl, cyclopent-2-en-1-yl, cyclopenta-2,4-dien-1-yl, cyclohexyl, cyclohex-2-en-1-yl, cycloheptyl, cyclooctanyl, 2,5-dimethylcyclopent-1-yl, 3,5-dichlorocyclohex-1-yl, 4-hydroxycyclohex-1-yl, 3,3,5-trimethylcyclohex-1-yl, octahydropentalenyl, octahydro-1H-indenyl, 3a,4,5,6,7,7a-hexahydro-3H-inden-4-yl, decahydroazulenyl, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

Non-limiting examples of alkenyl and alkenylene groups include straight, branched, and cyclic alkenyl groups. The olefin or olefins of an alkenyl group can be, for example, E, Z, cis, trans, terminal, or exo-methylene. An alkenyl or alkenylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkenyl and alkenylene groups include ethenyl, prop-1-en-1-yl, isopropenyl, but-1-en-4-yl; 2-chloroethenyl, 4-hydroxybuten-1-yl, 7-hydroxy-7-methyloct-4-en-2-yl, and 7-hydroxy-7-methyloct-3,5-dien-2-yl.

Non-limiting examples of alkynyl or alkynylene groups include straight, branched, and cyclic alkynyl groups. The triple bond of an alkylnyl or alkynylene group can be internal or terminal. An alkylnyl or alkynylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkynyl or alkynylene groups include ethynyl, prop-2-yn-1-yl, prop-1-yn-1-yl, and 2-methyl-hex-4-yn-1-yl; 5-hydroxy-5-methylhex-3-yn-1-yl, 6-hydroxy-6-methylhept-3-yn-2-yl, and 5-hydroxy-5-ethylhept-3-yn-1-yl.

A halo-alkyl group can be any alkyl group substituted with any number of halogen atoms, for example, fluorine, chlorine, bromine, and iodine atoms. A halo-alkenyl group can be any alkenyl group substituted with any number of halogen atoms. A halo-alkynyl group can be any alkynyl group substituted with any number of halogen atoms.

An alkoxy group can be, for example, an oxygen atom substituted with any alkyl, alkenyl, or alkynyl group. An ether or an ether group comprises an alkoxy group. Non-limiting examples of alkoxy groups include methoxy, ethoxy, propoxy, isopropoxy, and isobutoxy.

An aryl group can be heterocyclic or non-heterocyclic. An aryl group can be monocyclic or polycyclic. An aryl group can be substituted with any number of substituents described herein, for example, hydrocarbyl groups, alkyl groups, alkoxy groups, and halogen atoms. Non-limiting examples of aryl groups include phenyl, toluyl, naphthyl, pyrrolyl, pyridyl, imidazolyl, thiophenyl, and furyl. Non-limiting examples of substituted aryl groups include 3,4-dimethylphenyl, 4-tert-butylphenyl, 4-cyclopropylphenyl, 4-diethylaminophenyl, 4-(trifluoromethyl)phenyl, 4-(difluoromethoxy)-phenyl, 4-(trifluoromethoxy)phenyl, 3-chlorophenyl, 4-chlorophenyl, 3,4-dichlorophenyl, 2-fluorophenyl, 2-chlorophenyl, 2-iodophenyl, 3-iodophenyl, 4-iodophenyl, 2-methylphenyl, 3-fluorophenyl, 3-methylphenyl, 3-methoxyphenyl, 4-fluorophenyl, 4-methylphenyl, 4-methoxyphenyl, 2,3-difluorophenyl, 3,4-difluorophenyl, 3,5-difluorophenyl, 2,3-dichlorophenyl, 3,4-dichlorophenyl, 3,5-dichlorophenyl, 2-hydroxyphenyl, 3-hydroxyphenyl, 4-hydroxyphenyl, 2-methoxyphenyl, 3-methoxyphenyl, 4-methoxyphenyl, 2,3-dimethoxyphenyl, 3,4-dimethoxyphenyl, 3,5-dimethoxyphenyl, 2,4-difluorophenyl, 2,5-difluorophenyl, 2,6-difluorophenyl, 2,3,4-trifluorophenyl, 2,3,5-trifluorophenyl, 2,3,6-trifluorophenyl, 2,4,5-trifluorophenyl, 2,4,6-trifluorophenyl, 2,4-dichlorophenyl, 2,5-dichlorophenyl, 2,6-dichlorophenyl, 3,4-dichlorophenyl, 2,3,4-trichlorophenyl, 2,3,5-trichlorophenyl, 2,3,6-trichlorophenyl, 2,4,5-trichlorophenyl, 3,4,5-trichlorophenyl, 2,4,6-trichlorophenyl, 2,3-dimethylphenyl, 2,4-dimethylphenyl, 2,5-dimethylphenyl, 2,6-dimethylphenyl, 2,3,4-trimethylphenyl, 2,3,5-trimethylphenyl, 2,3,6-trimethylphenyl, 2,4,5-trimethylphenyl, 2,4,6-trimethylphenyl, 2-ethylphenyl, 3-ethylphenyl, 4-ethylphenyl, 2,3-diethylphenyl, 2,4-diethylphenyl, 2,5-diethylphenyl, 2,6-diethylphenyl, 3,4-diethylphenyl, 2,3,4-triethylphenyl, 2,3,5-triethylphenyl, 2,3,6-triethylphenyl, 2,4,5-triethylphenyl, 2,4,6-triethylphenyl, 2-isopropylphenyl, 3-isopropylphenyl, and 4-isopropylphenyl.

Non-limiting examples of substituted aryl groups include 2-aminophenyl, 2-(N-methylamino)phenyl, 2-(N,N-dimethylamino)phenyl, 2-(N-ethylamino)phenyl, 2-(N,N-diethylamino)phenyl, 3-aminophenyl, 3-(N-methylamino)phenyl, 3-(N,N-dimethylamino)phenyl, 3-(N-ethylamino)phenyl, 3-(N,N-diethylamino)phenyl, 4-aminophenyl, 4-(N-methylamino)phenyl, 4-(N,N-dimethylamino)phenyl, 4-(N-ethylamino)phenyl, and 4-(N,N-diethylamino)phenyl.

A heterocycle can be any ring containing a ring atom that is not carbon, for example, N, O, S, P, Si, B, or any other heteroatom. A heterocycle can be substituted with any number of substituents, for example, alkyl groups and halogen atoms. A heterocycle can be aromatic (heteroaryl) or non-aromatic. Non-limiting examples of heterocycles include pyrrole, pyrrolidine, pyridine, piperidine, succinamide, maleimide, morpholine, imidazole, thiophene, furan, tetrahydrofuran, pyran, and tetrahydropyran.

Non-limiting examples of heterocycles include: heterocyclic units having a single ring containing one or more heteroatoms, non-limiting examples of which include, diazirinyl, aziridinyl, azetidinyl, pyrazolidinyl, imidazolidinyl, oxazolidinyl, isoxazolinyl, thiazolidinyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl, 2,3,4,5-tetrahydro-1H-azepinyl, 2,3-dihydro-1H-indole, and 1,2,3,4-tetrahydroquinoline; and ii) heterocyclic units having 2 or more rings one of which is a heterocyclic ring, non-limiting examples of which include hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, and decahydro-1H-cycloocta[b]pyrrolyl.

Non-limiting examples of heteroaryl include: i) heteroaryl rings containing a single ring, non-limiting examples of which include, 1,2,3,4-tetrazolyl, [1,2,3]triazolyl, [1,2,4]triazolyl, triazinyl, thiazolyl, 1H-imidazolyl, oxazolyl, isoxazolyl, isothiazolyl, furanyl, thiophenyl, pyrimidinyl, 2-phenylpyrimidinyl, pyridinyl, 3-methylpyridinyl, and 4-dimethylaminopyridinyl; and ii) heteroaryl rings containing 2 or more fused rings one of which is a heteroaryl ring, non-limiting examples of which include: 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 4,5,6,7-tetrahydro-1-H-indolyl, quinoxalinyl, quinazolinyl, quinolinyl, 8-hydroxy-quinolinyl, and isoquinolinyl.

Any compound herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

### Pharmaceutically-Acceptable Salts.

The invention provides the use of pharmaceutically-acceptable salts of any therapeutic compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the invention. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the invention. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrrazole, pipyrrazole, imidazole, pyrazine, or pipyrazine.

In some embodiments, an ammonium salt is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrrazole salt, a pipyrrazole salt, an imidazole salt, a pyrazine salt, or a pipyrazine salt.

Acid addition salts can arise from the addition of an acid to a compound of the invention. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a methanesulfonate (mesylate) salt, an ethanesulfonate salt, a benzenesulfonate salt, a p-toluenesulfonate salt, a citrate salt, an oxalate salt, or a maleate salt.

### Pharmaceutical Compositions of the Invention.

A pharmaceutical composition of the invention can be used, for example, before, during, or after treatment of a subject with, for example, another pharmaceutical agent.

Subjects can be, for example, elderly adults, adults, adolescents, pre-adolescents, children, toddlers, infants, neonates, and non-human animals. In some embodiments, a subject is a patient.

A pharmaceutical composition of the invention can be a combination of any pharmaceutical compounds described herein with other chemical components, such as carriers, stabilizers, diluents, dispersing agents, suspending agents, thickening agents, and/or excipients. The pharmaceutical composition facilitates administration of the compound to an organism. Pharmaceutical compositions can be administered in therapeutically-effective amounts as pharmaceutical compositions by various forms and routes including, for example, intravenous, subcutaneous, intramuscular, oral, parenteral, ophthalmic, subcutaneous, transdermal, nasal, vaginal, and topical administration. In some embodiments, a pharmaceutical composition can comprise a population of molecules of compounds disclosed herein, wherein at least 10% of the molecules in the population of molecules each independently comprise at least one deuterium atom.

A pharmaceutical composition can be administered in a local manner, for example, via injection of the compound directly into an organ, optionally in a depot or sustained release formulation or implant. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

For oral administration, pharmaceutical compositions can be formulated by combining the active compounds with pharmaceutically-acceptable carriers or excipients. Such carriers can be used to formulate liquids, gels, syrups, elixirs, slurries, or suspensions, for oral ingestion by a subject. Non-limiting examples of solvents used in an oral dissolvable formulation can include water, ethanol, isopropanol, saline, physiological saline, DMSO, dimethylformamide, potassium phosphate buffer, phosphate buffer saline (PBS), sodium phosphate buffer, 4-2-hydroxyethyl-1-piperazineethanesulfonic acid buffer (HEPES), 3-(N-morpholino)propanesulfonic acid buffer (MOPS), piperazine-N,N′-bis(2-ethanesulfonic acid) buffer (PIPES), and saline sodium citrate buffer (SSC). Non-limiting examples of co-solvents used in an oral dissolvable formulation can include sucrose, urea, cremaphor, DMSO, and potassium phosphate buffer.

Pharmaceutical preparations can be formulated for intravenous administration. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of the active compounds in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

The active compounds can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

The compounds of the invention can be applied topically to the skin, or a body cavity, for example, oral, vaginal, bladder, cranial, spinal, thoracic, or pelvic cavity of a subject. The compounds of the invention can be applied to an accessible body cavity.

The compounds can also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, and PEG. In suppository forms of the compositions, a low-melting wax such as a mixture of fatty acid glycerides, optionally in combination with cocoa butter, can be melted.

In practicing the methods of treatment or use provided herein, therapeutically-effective amounts of the compounds described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compounds used, and other factors. The compounds can be used singly or in combination with one or more therapeutic agents as components of mixtures.

Pharmaceutical compositions can be formulated using one or more physiologically-acceptable carriers comprising excipients and auxiliaries, which facilitate processing of the active compounds into preparations that can be used pharmaceutically. Formulations can be modified depending upon the route of administration chosen. Pharmaceutical compositions comprising a compound described herein can be manufactured, for example, by mixing, dissolving, emulsifying, encapsulating, entrapping, or compression processes.

The pharmaceutical compositions can include at least one pharmaceutically-acceptable carrier, diluent, or excipient and compounds described herein as free-base or pharmaceutically-acceptable salt form. Pharmaceutical compositions can contain solubilizers, stabilizers, tonicity enhancing agents, buffers and preservatives.

Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, and cachets. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the invention include liquid, powder, gel, nanosuspension, nanoparticle, microgel, aqueous or oily suspensions, emulsion, and any combination thereof.

Non-limiting examples of pharmaceutically-acceptable excipients suitable for use in the invention include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, and any combination thereof.

A composition of the invention can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements or, alternatively, has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

In some, a controlled release formulation is a delayed release form. A delayed release form can be formulated to delay a compound's action for an extended period of time. A delayed release form can be formulated to delay the release of an effective dose of one or more compounds, for example, for about 4, about 8, about 12, about 16, or about 24 hours.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16 or about 24 hours.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

Therapeutic agents described herein can be administered before, during, or after the occurrence of a disease or condition, and the timing of administering the composition containing a therapeutic agent can vary. For example, the compositions can be used as a prophylactic and can be administered continuously to subjects with a propensity to conditions or diseases in order to lessen a likelihood of the occurrence of the disease or condition. The compositions can be administered to a subject during or as soon as possible after the onset of the symptoms. The administration of the therapeutic agents can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein.

A compound can be administered as soon as is practical after the onset of a disease or condition is detected or suspected, and for a length of time necessary for the treatment of the disease, such as, for example, from about 1 month to about 3 months. In some embodiments, the length of time a compound can be administered can be about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, or about 10 years. The length of treatment can vary for each subject.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for injection can be presented in unit dosage form, for example, in ampoules, or in multi-dose containers with a preservative.

Pharmaceutical compositions provided herein, can be administered in conjunction with other therapies, for example, chemotherapy, radiation, surgery, anti-inflammatory agents, and selected vitamins. The other agents can be administered prior to, after, or concomitantly with the pharmaceutical compositions.

Depending on the intended mode of administration, the pharmaceutical compositions can be in the form of solid, semi-solid or liquid dosage forms, such as, for example, tablets, suppositories, pills, capsules, powders, liquids, suspensions, lotions, creams, or gels, for example, in unit dosage form suitable for single administration of a precise dosage.

For solid compositions, nontoxic solid carriers include, for example, pharmaceutical grades of mannitol, lactose, starch, magnesium stearate, sodium saccharin, talc, cellulose, glucose, sucrose, and magnesium carbonate.

Compounds can be delivered via liposomal technology. The use of liposomes as drug carriers can increase the therapeutic index of the compounds. Liposomes are composed of natural phospholipids, and can contain mixed lipid chains with surfactant properties (e.g., egg phosphatidylethanolamine). A liposome design can employ surface ligands for attaching to unhealthy tissue. Non-limiting examples of liposomes include the multilamellar vesicle (MLV), the small unilamellar vesicle (SUV), and the large unilamellar vesicle (LUV). Liposomal physicochemical properties can be modulated to optimize penetration through biological barriers and retention at the site of administration, and to reduce a likelihood of developing premature degradation and toxicity to non-target tissues. Optimal liposomal properties depend on the administration route: large-sized liposomes show good retention upon local injection, small-sized liposomes are better suited to achieve passive targeting. PEGylation reduces the uptake of the liposomes by the liver and spleen, and increases the circulation time, resulting in increased localization at the inflamed site due to the enhanced permeability and retention (EPR) effect. Additionally, liposomal surfaces can be modified to achieve selective delivery of the encapsulated drug to specific target cells. Non-limiting examples of targeting ligands include monoclonal antibodies, vitamins, peptides, and polysaccharides specific for receptors concentrated on the surface of cells associated with the disease.

Non-limiting examples of dosage forms suitable for use in the disclosure include liquid, elixir, nanosuspension, aqueous or oily suspensions, drops, syrups, and any combination thereof. Non-limiting examples of pharmaceutically-acceptable excipients suitable for use in the disclosure include granulating agents, binding agents, lubricating agents, disintegrating agents, sweetening agents, glidants, anti-adherents, anti-static agents, surfactants, anti-oxidants, gums, coating agents, coloring agents, flavoring agents, coating agents, plasticizers, preservatives, suspending agents, emulsifying agents, plant cellulosic material and spheronization agents, and any combination thereof.

Compositions of the invention can be packaged as a kit. In some embodiments, a kit includes written instructions on the administration/use of the composition. The written material can be, for example, a label. The written material can suggest conditions methods of administration. The instructions provide the subject and the supervising physician with the best guidance for achieving the optimal clinical outcome from the administration of the therapy. The written material can be a label. In some embodiments, the label can be approved by a regulatory agency, for example the U.S. Food and Drug Administration (FDA), the European Medicines Agency (EMA), or other regulatory agencies.

### Dosing.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are liquids in vials or ampoules. Aqueous suspension compositions can be packaged in single-dose non-reclosable containers. Multiple-dose reclosable containers can be used, for example, in combination with a preservative. Formulations for parenteral injection can be presented in unit dosage form, for example, in ampoules, or in multi-dose containers with a preservative.

A compound described herein can be present in a composition in a range of from about 1 mg to about 5000 mg; from about 100 mg to about 4000 mg; from about 10 mg to about 3500 mg; from about 5 mg to about 3000 mg, from about 10 mg to about 2500 mg, from about 50 mg to about 2000 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1000 mg. In some embodiments, the therapeutically-effective amount of the compound is from about 250 mg to about 2500 mg. In some embodiments, the therapeutically-effective amount of the compound is from about 500 mg to about 2000 mg. In some embodiments, the therapeutically-effective amount of the compound is from about 1000 mg to about 3000 mg. In some embodiments, the therapeutically-effective amount of the compound is from about 2000 mg to about 3000 mg.

A compound described herein can be present in a composition in an amount of about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg. In some embodiments, the therapeutically-effective amount of the compound is about 2250 mg, about 2500 mg, or about 3000 mg. In some embodiments, the therapeutically-effective amount of the compound is about 1,200 mg. In some embodiments, the therapeutically-effective amount of the compound is about 1,500 m. In some embodiments, the therapeutically-effective amount of the compound is about 2,000 mg. In some embodiments, the therapeutically-effective amount of the compound is about 2,500 mg. In some embodiments, the therapeutically-effective amount of the compound is about 3,000 mg. In some embodiments, the therapeutically-effective amount of the compound is about 3,500 mg.

In some embodiments, a dose can be expressed in terms of an amount of the drug divided by the mass of the subject, for example, milligrams of drug per kilograms of subject body mass. In some embodiments, a compound is administered in an amount ranging from about 5 mg/kg to about 50 mg/kg, 250 mg/kg to about 2000 mg/kg, about 10 mg/kg to about 800 mg/kg, about 50 mg/kg to about 400 mg/kg, about 100 mg/kg to about 300 mg/kg, or about 150 mg/kg to about 200 mg/kg. In some embodiments, the therapeutically-effective amount of the compound is about 50 mg/kg. In some embodiments, the therapeutically-effective amount of the compound is about 40 mg/kg. In some embodiments, the therapeutically-effective amount of the compound is about 30 mg/kg.

### METHODS OF USE

In some embodiments, compounds of the invention can be used to treat cancer in a subject. A compound of the invention can, for example, slow the proliferation of cancer cell lines, or kill cancer cells. Non-limiting examples of cancer that can be treated by a compound of the invention include: acute lymphoblastic leukemia, acute myeloid leukemia, adrenocortical carcinoma, AIDS-related cancers, AIDS-related lymphoma, anal cancer, appendix cancer, astrocytomas, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, skin cancers, skin carcinoma merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenstrom macroglobulinemia, Wilms tumor, platinum-resistant carcinoma, adenocarcinoma, and extensive-stage small cell lung cancer (ES-SCLC). In some embodiments, the compounds of the invention show non-lethal toxicity.

Disclosed here in is a method comprising: a) administering a therapeutically-effective amount of a compound to a subject in need thereof, wherein the compound binds a mutant p53 protein and increases wild-type p53 activity of the mutant p53 protein; and b) after the administering, observing that the administering increases an immune response in the subject against a cancer. In some embodiments, the method comprises after the administering, obtaining a population of an immune cell in a biological sample obtained from the subject. In some embodiments, the biological sample is obtained from a tumor. In some embodiments, the administering increases a population of tumor infiltrating lymphocytes. In some embodiments, the administering increases a population of CD4+ T cells, CD8+ cells, T-regulatory T cells (Treg), and/or natural killer T (NKT) cells. In some embodiments, the administering decreases a population of g-MDSC or M2 macrophage cells. In some embodiments, the population of the immune cell is determined using a multiplexed immunofluorescence. In some embodiments, the population of the immune cell is determined using flow cytometry. In some embodiments, the population of the immune cell is determined using antibody staining.

In some embodiments, the method further comprises after the administering and before the observing, obtaining a tumor sample from the subject. In some embodiments, the method comprises observing the immune response at least one day after the administering.

A compound of the invention can increase the population of tumor infiltrating lymphocytes by at least or up to about 0.1%, at least or up to about 0.2%, at least or up to about 0.3%, at least or up to about 0.4%, at least or up to about 0.5%, at least or up to about 0.6%, at least or up to about 0.7%, at least or up to about 0.8%, at least or up to about 0.9%, at least or up to about 1%, at least or up to about 2%, at least or up to about 3%, at least or up to about 4%, at least or up to about 5%, at least or up to about 6%, at least or up to about 7%, at least or up to about 8%, at least or up to about 9%, at least or up to about 10%, at least or up to about 11%, at least or up to about 12%, at least or up to about 13%, at least or up to about 14%, at least or up to about 15%, at least or up to about 16%, at least or up to about 17%, at least or up to about 18%, at least or up to about 19%, at least or up to about 20%, at least or up to about 21%, at least or up to about 22%, at least or up to about 23%, at least or up to about 24%, at least or up to about 25%, at least or up to about 26%, at least or up to about 27%, at least or up to about 28%, at least or up to about 29%, at least or up to about 30%, at least or up to about 31%, at least or up to about 32%, at least or up to about 33%, at least or up to about 34%, at least or up to about 35%, at least or up to about 36%, at least or up to about 37%, at least or up to about 38%, at least or up to about 39%, at least or up to about 40%, at least or up to about 41%, at least or up to about 42%, at least or up to about 43%, at least or up to about 44%, at least or up to about 45%, at least or up to about 46%, at least or up to about 47%, at least or up to about 48%, at least or up to about 49%, at least or up to about 50%, as compared to the population of tumor cell lymphocytes in the absence of a compound of the invention.

Further disclosed herein is a method of treating a cancer comprising: a) administering a therapeutically-effective amount of a compound to a subject in need thereof, wherein the compound binds a mutant p53 protein and increases wild-type p53 activity of the mutant p53 protein; and b) after the administering, performing a gene expression assay on the subject, an increase is observed in a gene signature. In some embodiments, in the gene expression assay, an experiment is performed using a gene expression algorithm. In some embodiments, the gene expression algorithm comprises a NanoString nCounter gene expression algorithm. In some embodiments, the gene signature comprises an immune response gene signature. In some embodiments, the gene signature comprises a check-point biomarker signature.

In some embodiments, disclosed herein is a method of treating a cancer comprising: a) administering a therapeutically-effective amount of a compound to a subject in need thereof, wherein the compound binds a mutant p53 protein and increases wild-type p53 activity of the mutant p53 protein; and b) after the administering, performing a gene expression assay on the subject, wherein if in the gene expression assay, an experiment is performed using a gene expression algorithm, an increase is observed in a gene signature. In some embodiments, disclosed herein is a method of treating a cancer comprising: a) administering a therapeutically-effective amount of a compound to a subject in need thereof, wherein the compound binds a mutant p53 protein and increases wild-type p53 activity of the mutant p53 protein; and b) after the administering, performing a gene expression assay on the subject, wherein if in the gene expression assay, an experiment is performed using a NanoString gene expression algorithm, an increase is observed in a gene signature. In some embodiments, the gene signature comprises an immune response gene signature. In some embodiments, the gene signature comprises a check-point biomarker signature.

In some embodiments, the experiment comprises: a) obtaining a RNA sample of the subject, wherein the RNA sample comprises a plurality of target sequences, b) contacting the RNA sample with a plurality of reporter probes that are specific for the target sequence and a plurality of capture probes that are specific for the target sequence thereby providing a plurality of RNA-probe complexes, c) immobilizing the RNA-probe complexes on a substrate, and d) scanning the subject with a fluorescence microscope thereby counting the RNA-probe complexes on the substrate.

Further disclosed herein is a method comprising: a) determining that a subject is in need of an increase in a gene signature; and b) based on the determining, administering a therapeutically-effective amount of a compound to the subject in need thereof, wherein the compound binds a mutant p53 protein and increases wild-type p53 activity of the mutant p53 protein. In some embodiments, the gene signature comprises an immune response gene signature. In some embodiments, the gene signature comprises a check-point biomarker signature.

In some embodiments, the immune response gene signature is a tumor inflammation signature. In some embodiments, the immune response gene signature is a CD45 cell signature. In some embodiments, the immune response gene signature is a T-cell signature. In some embodiments, the immune response gene signature is an exhausted CD8 signature. In some embodiments, the check-point biomarker signature is a PDL1 signature. In some embodiments, the check-point biomarker signature is a PD-1 signature.

In some embodiments, the compound increases a stability of the mutant p53 protein. In some embodiments, the cancer expresses a mutant p53 protein. In some embodiments, the mutant p53 protein has a mutation at amino acid 220. In some embodiments, the mutant p53 protein is p53 Y220C. In some embodiments, the compound selectively binds the mutant p53 protein as compared to a wild type p53. In some embodiments, the cancer is ovarian cancer. In some embodiments, the cancer is breast cancer. In some embodiments, the cancer is lung cancer. In some embodiments, the subject is human.

In some embodiments, the administering of the compound is oral. In some embodiments, the administering of the compound is subcutaneous. In some embodiments, the administering of the compound is topical. In some embodiments, the administering is once daily. In some embodiments, the administering is twice daily. In some embodiments, the administering is for at least 2 days, at least 3 days, at least 4 days, at least 5 days, at least 6 days, at least about 1 week, at least about 2 weeks, at least about 3 weeks, at least 4 weeks, at least 5 weeks, at least 6 weeks, at least 7 weeks, at least 8 weeks, or more.

In some embodiments, the therapeutically-effective amount of the compound is from about 1 mg/kg to about 10 mg/kg, from about 1 mg/kg to about 20 mg/kg, from about 1 mg/kg to about 30 mg/kg, from about 1 mg/kg to about 40 mg/kg, from about 1 mg/kg to about 50 mg/kg, from about 1 mg/kg to about 60 mg/kg, from about 1 mg/kg to about 70 mg/kg, from about 1 mg/kg to about 80 mg/kg, from about 1 mg/kg to about 90 mg/kg, from about 1 mg/kg to about 100 mg/kg, from about 10 mg/kg to about 20 mg/kg, from about 10 mg/kg to about 40 mg/kg, from about 10 mg/kg to about 60 mg/kg, from about 10 mg/kg to about 80 mg/kg, from about 10 mg/kg to about 100 mg/kg, from about 15 mg/kg to about 20 mg/kg, from about 15 mg/kg to about 40 mg/kg, from about 15 mg/kg to about 60 mg/kg, from about 15 mg/kg to about 80 mg/kg, from about 15 mg/kg to about 100 mg/kg, from about 30 mg/kg to about 40 mg/kg, from about 30 mg/kg to about 60 mg/kg, from about 30 mg/kg to about 80 mg/kg, from about 30 mg/kg to about 100 mg/kg, from about 40 mg/kg to about 60 mg/kg, from about 40 mg/kg to about 80 mg/kg, from about 40 mg/kg to about 100 mg/kg, from about 50 mg/kg to about 100 mg/kg, from about 60 mg/kg to about 100 mg/kg, from about 70 mg/kg to about 100 mg/kg, or from about 80 mg/kg to about 100 mg/kg. In some embodiments, the therapeutically-effective amount of the compound is about 10 mg/kg. In some embodiments, the therapeutically-effective amount of the compound is about 15 mg/kg. In some embodiments, the therapeutically-effective amount of the compound is about 20 mg/kg. In some embodiments, the therapeutically-effective amount of the compound is about 30 mg/kg. In some embodiments, the therapeutically-effective amount of the compound is about 40 mg/kg. In some embodiments, the therapeutically-effective amount of the compound is about 50 mg/kg. In some embodiments, the therapeutically-effective amount of the compound is about 60 mg/kg.

In some embodiments, the subject has a tumor. In some embodiments, the tumor comprises a Y220C mutation. In some embodiments, the tumor is ovarian cancer. In some embodiments, the tumor is breast cancer. In some embodiments, the tumor is lung cancer. In some embodiments, the tumor is prostate cancer.

The methods of the disclosure can administer a compound or structure comprising a substituted heterocyclyl group. In some embodiments, the structure comprises a heterocyclyl group comprising a halo substituent. In some embodiments, the structure comprises an indole group. In some embodiments, the indole group comprises a propargyl substituent at a 2-position of the indole group. In some embodiments, the propargyl substituent is attached to the indole group via an sp carbon atom of the propargyl substituent. In some embodiments, the propargyl substituent is attached to a nitrogen atom of an aniline group via a methylene group of the propargyl substituent. In some embodiments, the indole group comprises an amino substituent at a 4-position of the indole group. In some embodiments, the amino substituent is attached to the heterocyclyl group.

In some embodiments, the compound is of the formula:

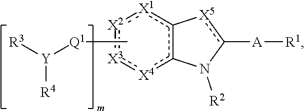

wherein:
each [symbol] is independently a single bond or a double bond;
X¹ is CR⁵, CR⁵R⁶, N, NR⁵, O, S, C═O, C═S, or a carbon atom connected to Q¹;
X² is CR⁷, CR⁷R⁸, N, NR⁷, O, S, C═O, C═S, or a carbon atom connected to Q¹;
X³ is CR⁹, CR⁹R¹⁰, N, NR⁹, O, S, C═O, C═S, or a carbon atom connected to Q¹;
X⁴ is CR¹¹, CR¹¹R¹², N, NR¹¹, O, S, C═O, C═S, or a carbon atom connected to Q¹;
X⁵ is CR¹³, N, or NR¹³;
   wherein at least one of X¹, X², X³, and X⁴ is a carbon atom connected to Q¹;
A is a linking group;
Q¹ is alkylene, alkenylene, or alkynylene, each of which is independently substituted or unsubstituted, C═O, C═S, C═CR¹⁴R¹⁵, C═NR¹⁴, or a bond;
m is 1, 2, 3, or 4;
Y is N, O, or absent;
R¹ is alkyl, alkenyl, alkynyl, alkoxy, aryl, heteroaryl, or heterocyclyl, each of which is independently substituted or unsubstituted, or —C(O)R¹⁶, —C(O)OR¹⁶, —C(O)NR¹⁶R¹⁷, —OR¹⁶, —SR¹⁶, —NR¹⁶R¹⁷, —NR¹⁶C(O)R¹⁶, —OC(O)R¹⁶, —SiR¹⁶R¹⁷R¹⁸, or hydrogen or halogen;
each R³ and R⁴ is independently —C(O)R¹⁹, —C(O)OR¹⁹, —C(O)NR¹⁹R²⁰, —SOR¹⁹, —SO₂R¹⁹, alkyl, alkenyl, alkynyl, aryl, heteroaryl, or heterocyclyl, each of which is independently substituted or unsubstituted, or hydrogen, or R³ and R⁴ together with the nitrogen atom to which R³ and R⁴ are bound form a ring, wherein the ring is substituted or unsubstituted, or R³ is absent;
each R², R⁵, R⁶, R⁷, R⁸, R⁹, R¹⁰, R¹¹, R¹, R¹³, R¹⁴, R¹, R¹⁶, R¹⁷, and R¹⁸ is independently —C(O)R²¹, —C(O)OR²¹, —C(O)NR²¹R²², —OR²¹, —SR²¹, —NR²¹R²², —NR²¹C(O)R²², —OC(O)R²¹, alkyl, alkenyl, alkynyl, aryl, heteroaryl, or heterocyclyl, each of which is independently substituted or unsubstituted, or hydrogen or halogen;
each R¹⁹ and R²⁰ is independently —C(O)R²³, —C(O)OR²³, —C(O)NR²³R²⁴, —OR²³, —SR²³, —NR²³R²⁴, —NR²³C(O)R²⁴, —OC(O)R²³, alkyl, alkenyl, alkynyl, aryl, heteroaryl, or heterocyclyl, each of which is independently substituted or unsubstituted, or hydrogen or halogen;
each R²¹ and R²² is independently alkyl, alkenyl, alkynyl, aryl, heteroaryl, or heterocyclyl, each of which is independently substituted or unsubstituted, or hydrogen; and
each R²³ and R²⁴ is independently alkyl, alkenyl, alkynyl, aryl, heteroaryl, or heterocyclyl, each of which is independently substituted or unsubstituted, or hydrogen,
   or a pharmaceutically-acceptable salt thereof.

In some embodiments, A is alkylene, alkenylene, or alkynylene, each of which is substituted or unsubstituted. In some embodiments, A is aryl, heteroaryl, or heterocyclyl, each of which is substituted or unsubstituted. In some embodiments, the compound is of the formula:

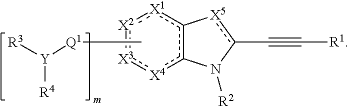

In some embodiments, Q¹ is C₁-alkylene. In some embodiments, Q¹ is a bond. In some embodiments, m is 1. In some embodiments, m is 2. In some embodiments, Y is N. In some embodiments, Y is O. In some embodiments, each R³ and R⁴ is independently alkyl, alkylene, alkenyl, alkenylene, alkynyl, aryl, heteroaryl, or heterocyclyl, each of which is independently substituted or unsubstituted; or hydrogen. In some embodiments, R³ is alkyl, alkylene, alkenyl, alkenylene, alkynyl, each of which is independently substituted or unsubstituted; and R⁴ is aryl, heteroaryl, or heterocyclyl, each of which is independently substituted or unsubstituted. In some embodiments, R³ is H; and R⁴ is aryl, heteroaryl, or heterocyclyl, each of which is independently substituted or unsubstituted. R¹³ is hydrogen.

In some embodiments, the compound is of the formula:

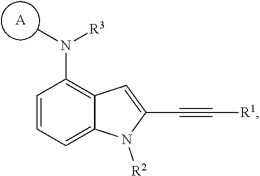

wherein ring A is a cyclic group that is substituted or unsubstituted. In some embodiments, R² is substituted or unsubstituted alkyl. In some embodiments, R² is methyl, ethyl, propyl, iso-propyl, butyl, or tert-butyl, each of which is substituted or unsubstituted. In some embodiments, R² is substituted ethyl. In some embodiments, R² is trifluoroethyl.

In some embodiments, the compound is of the formula:

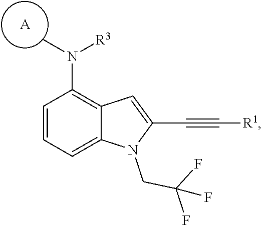

In some embodiments, ring A is aryl, heteroaryl, or heterocyclyl, each of which is substituted or unsubstituted. In some embodiments, ring A is substituted aryl. In some embodiments, ring A is substituted heteroaryl. In some embodiments, ring A is substituted heterocyclyl.

In some embodiments, R¹ is alkyl, alkenyl, —C(O)R¹⁶, —C(O)OR¹⁶, or —C(O)NR¹⁶R¹⁷, each of which is unsubstituted or substituted. In some embodiments, R¹ is substituted alkyl. In some embodiments, R¹ is alkyl substituted with NR¹⁶R¹⁷. In some embodiments, the compound is of the formula:

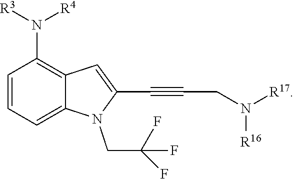

In some embodiments, each R¹⁶ and R¹⁷ is independently alkyl, alkenyl, aryl, heteroaryl, heterocyclyl, each of which is independently substituted or unsubstituted; or hydrogen. In some embodiments, R¹⁶ is hydrogen or alkyl. In some embodiments, R¹⁷ is aryl, heteroaryl, or heterocyclyl, each of which is independently substituted or unsubstituted. In some embodiments, R¹⁷ is substituted aryl. In some embodiments, R¹⁷ is substituted phenyl. In some embodiments, R¹⁷ is phenyl substituted with a sulfoxide group, carboxyl group, amide group, amino group, alkyl, alkoxy, hydroxy, or heterocyclyl, each of which is independently substituted or unsubstituted, or halo or cyano. In some embodiments, R¹⁷ is phenyl substituted with methoxy. In some embodiments, R¹⁷ is phenyl substituted with a substituted sulfoxide group. In some embodiments, R¹⁷ is phenyl substituted with a carboxyl group. In some embodiments, R¹⁷ is phenyl substituted with an amide group.

In some embodiments, the compound is 4-[(3-{4-[(1,5-dihydroxypentan-3-yl)amino]-1-(2,2,2-trifluoroethyl)-1H-indol-2-yl}prop-2-yn-1-yl)amino]-3-methoxybenzene-1-sulfonamide. In some embodiments, the compound is 2-(3-((2-methoxy-4-(methylsulfonyl)phenyl)amino)prop-1-yn-1-yl)-N-((1r,4r)-4-morpholinocyclohexyl)-1-(oxiran-2-ylmethyl)-1H-indol-4-amine. In some embodiments, the compound is 3-methoxy-4-({3-[4-({2-oxaspiro[3.3]heptan-6-yl}amino)-1-(2,2,2-trifluoroethyl)-1H-indol-2-yl]prop-2-yn-1-yl}amino)benzene-1-sulfonamide. In some embodiments, the compound is 4-((3-(4-(((3S,4R)-3-fluoro-1-methylpiperidin-4-yl)amino)-1-(2,2,2-trifluoroethyl)-1H-indol-2-yl)prop-2-yn-1-yl)amino)-3-methoxy-N-methylbenzamide. In some embodiments, the compound is N-(2,3-dihydroxypropyl)-4-{[3-(4-{[(3S,4R)-3-fluoro-1-methylpiperidin-4-yl]amino}-1-(2,2,2-trifluoroethyl)-1H-indol-2-yl)prop-2-yn-1-yl]amino}-3-methoxybenzamide. In some embodiments, the compound is 3-methoxy-N-(2-methoxyethyl)-N-methyl-4-((3-(4-((tetrahydro-2H-pyran-4-yl)amino)-1-(2,2,2-trifluoroethyl)-1H-indol-2-yl)prop-2-yn-1-yl)amino)benzenesulfonamide. In some embodiments, the compound is N-(2,3-dihydroxypropyl)-4-((3-(4-((1,1-dioxidotetrahydro-2H-thiopyran-4-yl)amino)-1-(2,2,2-trifluoroethyl)-1H-indol-2-yl)prop-2-yn-1-yl)amino)-3-methoxybenzenesulfonamide. In some embodiments, the compound is 3-methoxy-4-((3-(4-(3-(1-methylpiperidin-4-yl)ureido)-1-(2,2,2-trifluoroethyl)-1H-indol-2-yl)prop-2-yn-1-yl)amino)benzamide. In some embodiments, the compound is N-((3S,4R)-3-fluoropiperidin-4-yl)-2-(3-((2-methoxy-4-(methylsulfonyl)phenyl)amino)prop-1-yn-1-yl)-1-(2,2,2-trifluoroethyl)-1H-indol-4-amine.

In some embodiments, the method further comprises administering to the subject a therapeutically-effective amount of an anti-cancer agent. In some embodiments, the anti-cancer agent is an immune checkpoint inhibitor. In some embodiments, the immune checkpoint inhibitor is an anti-PD-1 agent. In some embodiments, the immune checkpoint inhibitor is an anti-PD-L1 agent. In some embodiments, the administering the anti-cancer agent is oral. In some embodiments, the administering the anti-cancer agent is intravenous. In some embodiments, the therapeutically-effective amount of the anti-cancer agent is from about 1 mg to about 500 mg. In some embodiments, the therapeutically-effective amount of the anti-cancer agent is from about 20 mg to about 100 mg. In some embodiments, the therapeutically-effective amount of the anti-cancer agent is from about 20 mg to about 400 mg. In some embodiments, the therapeutically-effective amount of the anti-cancer agent is from about 50 mg to about 400 mg. In some embodiments, the therapeutically-effective amount of the anti-cancer agent is from about 100 mg to about 400 mg. In some embodiments, the therapeutically-effective amount of the anti-cancer agent is from about 200 mg to about 400 mg. In some embodiments, the therapeutically-effective amount of the anti-cancer agent is from about 200 mg to about 500 mg. In some embodiments, the therapeutically-effective amount of the anti-cancer agent is from about 500 mg to about 2000 mg. In some embodiments, the therapeutically-effective amount of the anti-cancer agent is about 150 mg, about 200 mg, about 250 mg, about 300 mg, about 350 mg, about 400 mg, or about 500 mg.

In some embodiments, the therapeutically-effective amount of the anti-cancer agent is from about 1 μg/kg to about 10 mg/kg. In some embodiments, the therapeutically-effective amount of the anti-cancer agent is from about 10 μg/kg to about 500 μg/kg. In some embodiments, the therapeutically-effective amount of the anti-cancer agent is from about 1 mg/kg to about 10 mg/kg. In some embodiments, the therapeutically-effective amount of the anti-cancer agent is from about 2 mg/kg to about 6 mg/kg.

In some embodiments, the method comprises after the observing, further administering the therapeutically-effective amount of the compound to the subject. In some embodiments, the method comprises after the observing, discontinuing the administering the compound to the subject. In some embodiments, the method further comprises administering a second therapeutically-effective amount of the compound. In some embodiments, the second therapeutically-effective amount of the compound is less than the therapeutically-effective amount of the compound. In some embodiments, the method comprises discontinuing administering of the compound to the subject. In some embodiments, if the administering the therapeutically-effective amount of the compound does not modulate the expression level or population of the immune cell in the tumor, the second therapeutically-effective amount of the compound is the same as the therapeutically-effective amount of the compound. In some embodiments, if the administering the therapeutically-effective amount of the compound does not modulate the expression level or population of the immune cell in the tumor, the second therapeutically-effective amount of the compound is greater than the therapeutically-effective amount of the compound.

### EXAMPLES

### Example 1: Compounds of the Disclosure

Indole compounds with alkynyl, aryl, and heteroaryl linkers were prepared. Alkynyl-linked indole compounds are shown in TABLE 1. Aryl-linked indole compounds are shown in TABLE 2. Heteroaryl-linked indole compounds are shown in TABLE 3. Compounds substituted with at least one deuterium atom are shown in TABLE 4. The disclosure provides these compounds and a pharmaceutically-acceptable salt thereof.

TABLE 1
Alkynyl indole compounds of the disclosure.

| Mol # | IUPAC name |
|---|---|
| 1. | 1-Anilino-3-{1-ethyl-5-[(1-methyl-4-piperidylamino)methyl]-1H-indol-2-yl}-2-propyne |
| 2. | 1-Anilino-3-{1-ethyl-5-[(methylamino)methyl]-1H-indol-2-yl}-2-propyne |
| 3. | 1-Anilino-3-{1-ethyl-5-[(tetrahydro-2H-pyran-4-ylamino)methyl]-1H-indol-2-yl}-2- |
| | propyne |
| 4. | 1-Anilino-3-[5-(benzylaminomethyl)-1-ethyl-1H-indol-2-yl]-2-propyne |
| 5. | 3-{1-Ethyl-5-[(methylamino)methyl]-1H-indol-2-yl}-1-(p-fluorophenylamino)-2-propyne |
| 6. | 3-{1-Ethyl-5-[(tetrahydro-2H-pyran-4-ylmino)methyl]-1H-indol-2-yl}-1-(p- |
| | fluorophenylamino)-2-propyne |
| 7. | 1-(p-Chlorophenylamino)-3-{1-ethyl-5-[(1-methyl-4-piperidylamino)methyl]-1H-indol-2- |
| | yl}-2-propyne |
| 8. | 3-{1-Ethyl-5-[(tetrahydro-2H-pyran-4-ylamino)methyl]-1H-indol-2-yl}-1-(6-methyl-3- |
| | pyridylamino)-2-propyne |
| 9. | 3-{1-Ethyl-5-[(methylamino)methyl]-1H-indol-2-yl}-1-(6-methyl-3-pyridylamino)-2- |
| | propyne |
| 10. | 3-{1-Ethyl-5-[(1-methyl-4-piperidylamino)methyl]-1H-indol-2-yl}-1-(2-methyl-4- |
| | pyridylamino)-2-propyne |
| 11. | 3-[5-(Benzylaminomethyl)-1-ethyl-1H-indol-2-yl]-1-(2-methyl-4-pyridylamino)-2- |
| | propyne |
| 12. | N-(3-{5-[(Diethylamino)methyl]-1-ethyl-1H-indol-2-yl}prop-2-yn-1-yl)aniline |
| 13. | 4-Chloro-N-(3-{5-[(diethylamino)methyl]-1-ethyl-1H-indol-2-yl}prop-2-yn-1-yl)aniline |
| 14. | N-({1-Ethyl-2-[3-(phenylamino)prop-1-yn-1-yl]-1H-indol-5-yl}methyl)oxetan-3-amine |
| 15. | N-[3-(1-Ethyl-5-{[(2-methylpropyl)amino]methyl}-1H-indol-2-yl)prop-2-yn-1-yl]aniline |
| 16. | N-[3-(1-Ethyl-5-{[(2-methoxyethyl)amino]methyl}-1H-indol-2-yl)prop-2-yn-1-yl]aniline |
| 17. | N-({1-ethyl-2-[3-(phenylamino)prop-1-yn-1-yl]-1H-indol-5-yl}methyl)-1- |
| | methanesulfonylpiperidin-4-amine |
| 18. | N-(3-{1-Ethyl-5-[(ethylamino)methyl]-1H-indol-2-yl}prop-2-yn-1-yl)aniline |
| 19. | N-{3-[5-({[2-(Dimethylamino)ethyl]amino}methyl)-1-ethyl-1H-indol-2-yl]prop-2-yn-1- |
| | yl}aniline |
| 20. | 6-tert-Butyl-N-[3-(1-ethyl-5-{[(1-methylpiperidin-4-yl)amino]methyl}-1H-indol-2- |
| | yl)prop-2-yn-1-yl]pyridin-3-amine |
| 21. | N-[(2-{3-[(4-Chlorophenyl)amino]prop-1-yn-1-yl}-1-ethyl-1H-indol-5-yl)methyl]oxan-4- |
| | amine |
| 22. | 6-tert-Butyl-N-(3-{1-ethyl-5-[(methylamino)methyl]-1H-indol-2-yl}prop-2-yn-1- |
| | yl)pyridin-3-amine |
| 23. | 4-[(3-{1-Ethyl-5-[(methylamino)methyl]-1H-indol-2-yl}prop-2-yn-1- |
| | yl)amino]benzonitrile |
| 24. | 4-tert-Butyl-N-(3-{1-ethyl-5-[(methylamino)methyl]-1H-indol-2-yl}prop-2-yn-1- |
| | yl)benzamide |
| 25. | 4-Chloro-N-(3-{1-ethyl-5-[(methylamino)methyl]-1H-indol-2-yl}prop-2-yn-1-yl)-3- |
| | fluorobenzamide |
| 26. | 4-Cyano-N-({1-ethyl-2-[3-(phenylformamido)prop-1-yn-1-yl]-1H-indol-5-yl}methyl)-N- |
| | methylbenzamide |
| 27. | 3-(3-{1-Ethyl-5-[(methylamino)methyl]-1H-indol-2-yl}prop-2-yn-1-yl)-1-[4- |
| | (trifluoromethyl)phenyl]urea |
| 28. | N-{[1-(2-Chloroethyl)-2-{3-[(4-chlorophenyl)amino]prop-1-yn-1-yl}-1H-indol-5- |
| | yl]methyl}oxan-4-amine |
| 29. | 2-(4-{[3-(1-Ethyl-5-{[(1-methylpiperidin-4-yl)amino]methyl}-1H-indol-2-yl)prop-2-yn- |
| | 1-yl]amino}phenyl)-2-methylpropanenitrile |
| 30. | 4-Cyano-N-(3-{1-ethyl-5-[(methylamino)methyl]-1H-indol-2-yl}prop-2-yn-1- |
| | yl)benzamide |
| 31. | N-(3-{1-Ethyl-5-[(methylamino)methyl]-1H-indol-2-yl}prop-2-yn-1-yl)-6- |
| | methylpyridine-3-carboxamide |
| 32. | 3-[3-(1-Ethyl-5-{[(1-methylpiperidin-4-yl)amino]methyl}-1H-indol-2-yl)prop-2-yn-1-yl]- |
| | 1-phenylurea |
| 33. | N-[(2-{3-[(4-Chloro-3-fluorophenyl)amino]prop-1-yn-1-yl}-1-ethyl-1H-indol-5- |
| | yl)methyl]-1-methylpiperidin-4-amine |
| 34. | 2-(5-{[3-(1-Ethyl-5-{[(1-methylpiperidin-4-yl)amino]methyl}-1H-indol-2-yl)prop-2-yn- |
| | 1-yl]amino}pyridin-2-yl)-2-methylpropanenitrile |
| 35. | N-{[1-(2-Chloroethyl)-2-{3-[(4-chlorophenyl)amino]prop-1-yn-1-yl}-1H-indol-5- |
| | yl]methyl}-1-methylpiperidin-4-amine |
| 36. | 6-tert-Butyl-N-[3-(1-ethyl-5-{[(1-methanesulfonylpiperidin-4-yl)amino]methyl}-1H- |
| | indol-2-yl)prop-2-yn-1-yl]pyridin-3-amine |
| 37. | 2-(4-{[3-(1-Ethyl-5-{[(1-methylpiperidin-4-yl)amino]methyl}-1H-indol-2-yl)prop-2-yn- |
| | 1-yl]amino}phenyl)-2-methylpropanoic acid |
| 38. | 3-(1-Ethyl-5-{[(1-methylpiperidin-4-yl)amino]methyl}-1H-indol-2-yl)-N-methylprop-2- |
| | ynamide |
| 39. | Ethyl 2-(4-{[3-(1-ethyl-5-{[(1-methylpiperidin-4-yl)amino]methyl}-1H-indol-2-yl)prop- |
| | 2-yn-1-yl]amino}phenyl)-2-methylpropanoate |
| 40. | 2-(5-{[3-(1-Ethyl-5-{[(oxan-4-yl)amino]methyl}-1H-indol-2-yl)prop-2-yn-1- |
| | yl]amino}pyridin-2-yl)-2-methylpropanenitrile |
| 41. | N-[(1-Ethyl-2-{3-[(4-methylphenyl)amino]prop-1-yn-1-yl}-1H-indol-5-yl)methyl]-1- |
| | methylpiperidin-4-amine |
| 42. | 4-{[3-(1-Ethyl-5-{[(1-methylpiperidin-4-yl)amino]methyl}-1H-indol-2-yl)prop-2-yn-1- |
| | yl]amino}benzonitrile |
| 43. | 3-(1-Ethyl-5-{[(1-methylpiperidin-4-yl)amino]methyl}-1H-indol-2-yl)-N-phenylprop-2- |
| | ynamide |
| 44. | N-[(2-{3-[(4-Chlorophenyl)amino]prop-1-yn-1-yl}-1-ethyl-1H-indol-5-yl)methyl]-1- |
| | methanesulfonylpiperidin-4-amine |
| 45. | 1-(4-{[(2-{3-[(4-Chlorophenyl)amino]prop-1-yn-1-yl}-1-ethyl-1H-indol-5- |
| | yl)methyl]amino}piperidin-1-yl)ethan-1-one |
| 46. | 6-tert-Butyl-N-[3-(1-ethyl-5-{[(1-methylpiperidin-4-yl)amino]methyl}-1H-indol-2- |
| | yl)prop-2-yn-1-yl]pyridine-3-carboxamide |
| 47. | N-(3-{1-Ethyl-5-[(methylamino)methyl]-1H-indol-2-yl}prop-2-yn-1-yl)-4- |
| | (trifluoromethyl)aniline |
| 48. | N-[(1-Ethyl-2-{3-[(4-methylphenyl)amino]prop-1-yn-1-yl}-1H-indol-5-yl)methyl]oxan- |
| | 4-amine |
| 49. | N-(3-{1-ethyl-4-[(methylamino)methyl]-1H-indol-2-yl}prop-2-yn-1-yl)aniline |
| 50. | N-[3-(1-ethyl-4-{[4-(pyrrolidin-1-yl)piperidin-1-yl]methyl}-1H-indol-2-yl)prop-2-yn-1- |
| | yl]aniline |
| 51. | N-({1-ethyl-2-[3-(phenylamino)prop-1-yn-1-yl]-1H-indol-4-yl}methyl)-1- |
| | methylpiperidin-4-amine |
| 52. | 1-[(2-{3-[(4-chlorophenyl)arnino]prop-1-yn-1-yl}-1-ethyl-1H-indol-4- |
| | yl)methyl]piperidin-4-ol |
| 53. | 4-Chloro-N-[3-(1-ethyl-4-{[4-(pyrrolidin-1-yl)piperidin-1-yl]methyl}-1H-indol-2- |
| | yl)prop-2-yn-1-yl]aniline |
| 54. | 1-[(2-{3-[(4-chlorophenyl)amino]prop-1-yn-1-yl}-1-ethyl-1H-indol-4-yl)methyl]-N,N- |
| | dimethylpiperidin-4-amine |
| 55. | 4-Chloro-N-(3-{1-ethyl-4-[(4-methylpiperazin-1-yl)methyl]-1H-indol-2-yl}prop-2-yn-1- |
| | yl)aniline |
| 56. | 1-{1-[(2-{3-[(4-Chlorophenyl)amino]prop-1-yn-1-yl}-1-ethyl-1H-indol-4- |
| | yl)methyl]piperidin-4-yl}piperidin-4-ol |
| 57. | 2-(5-{[3-(4-{[4-(4-Aminopiperidin-1-yl)piperidin-1-yl]methyl}-1-ethyl-1H-indol-2- |
| | yl)prop-2-yn-1-yl]amino]pyridin-2-yl)-2-methylpropanenitrile |
| 58. | 1-[(1-ethyl-2-{3-[(4-fluorophenyl)amino]prop-1-yn-1-yl}-1H-indol-5-yl)methyl]-N,N- |
| | dimethylpiperidin-4-amine |
| 59. | 4-N-({1-ethyl-2-[3-(phenylamino)prop-1-yn-1-yl]-1H-indol-5-yl}methyl)-1-N,1-N- |
| | dimethylcyclohexane-1,4-diamine |
| 60. | 4-chloro-N-(3-{1-ethyl-5-[(methylamino)methyl]-1H-indol-2-yl}prop-2-yn-1-yl)-3- |
| | fluoroaniline |
| 61. | 6-tert-butyl-N-(3-{1-ethyl-5-[(methylamino)methyl]-1H-indol-2-yl}prop-2-yn-1- |
| | yl)pyridine-3-carboxamide |
| 62. | N-(3-{1-ethyl-5-[(methylamino)methyl]-1H-indol-2-yl}prop-2-yn-1-yl)benzamide |
| 63. | 3-(3-{1-ethyl-5-[(methylamino)methyl]-1H-indol-2-yl}prop-2-yn-1-yl)-1-(4- |
| | methylphenyl)urea |
| 64. | 4-chloro-N-(3-{1-ethyl-5-[(methylamino)methyl]-1H-indol-2-yl}prop-2-yn-1-yl)aniline |
| 65. | 4-{[3-(1-ethyl-5-{[(oxan-4-yl)amino]methyl}-1H-indol-2-yl)prop-2-yn-1- |
| | yl]amino}benzonitrile |
| 66. | N-[(2-{3-[(4-chloro-3-fluorophenyl)amino]prop-1-yn-1-yl}-1-ethyl-1H-indol-5- |
| | yl)methyl]oxan-4-amine |
| 67. | 3-[3-(1-ethyl-5-{[(oxan-4-yl)amino]methyl}-1H-indol-2-yl)prop-2-yn-1-yl]-1-phenylurea |
| 68. | 6-tert-butyl-N-[3-(1-ethyl-5-{[(oxan-4-yl)amino]methyl}-1H-indol-2-yl)prop-2-yn-1- |
| | yl]pyridin-3-amine |
| 69. | 4-{[(2-{3-[(4-chlorophenyl)amino]prop-1-yn-1-yl}-1-ethyl-1H-indol-5- |
| | yl)methyl]amino}-1λ⁶-thiane-1,1-dione |
| 70. | N-[(2-{3-[(4-chlorophenyl)amino]prop-1-yn-1-yl}-1-ethyl-1H-indol-5-yl)methyl]-1-(2- |
| | methanesulfonylethyl)piperidin-4-amine |
| 71. | 1-(4-{[(2-{3-[(4-chlorophenyl)amino]prop-1-yn-1-yl}-1-ethyl-1H-indol-5- |
| | yl)methyl]amino}piperidin-1-yl)-2-(dimethylamino)ethan-1-one |
| 72. | 2-(4-{[(2-{3-[(4-chlorophenyl)amino]prop-1-yn-1-yl}-1-ethyl-1H-indol-5- |
| | yl)methyl]amino}piperidin-1-yl)-N,N-dimethylacetamide |
| 73. | 2-tert-butyl-N-[3-(1-ethyl-5-{[(1-methylpiperidin-4-yl)amino]methyl}-1H-indol-2- |
| | yl)prop-2-yn-1-yl]pyrimidin-5-amine |
| 74. | 2-methyl-2-(5-{[3-(5-{[(oxan-4-yl)amino]methyl}-1-(2,2,2-trifluoroethyl)-1H-indol-2- |
| | yl)prop-2-yn-1-yl]amino}pyridin-2-yl)propanenitrile |
| 75. | 2-[5-({3-[1-(2-fluoroethyl)-5-{[(oxan-4-yl)amino]methyl}-1H-indol-2-yl]prop-2-yn-1- |
| | yl}amino)pyridin-2-yl]-2-methylpropanenitrile |
| 76. | 3-(1-ethyl-5-{[(1-methylpiperidin-4-yl)amino]methyl}-1H-indol-2-yl)prop-2-yn-1-ol |
| 77. | 2-[5-({3-[1-(2-chloroethyl)-5-{[(oxan-4-yl)amino]methyl}-1H-indol-2-yl]prop-2-yn-1- |
| | yl}amino)pyridin-2-yl]-2-methylpropanenitrile |
| 78. | 2-[5-({3-[1-(2,2-difluoroethyl)-5-{[(oxan-4-yl)amino]methyl}-1H-indol-2-yl]prop-2-yn- |
| | 1-yl}amino)pyridin-2-yl]-2-methylpropanenitrile |
| 79. | 6-chloro-N-[3-(1-ethyl-5-{[(1-methylpiperidin-4-yl)amino]methyl}-1H-indol-2-yl)prop- |
| | 2-yn-1-yl]pyridin-3-amine |
| 80. | tert-butyl N-({3-ethyl-2-[3-(phenylamino)prop-1-yn-1-yl]-1H-indol-6-yl}methyl)-N- |
| | (oxan-4-yl)carbamate |
| 81. | 6-Chloro-N-[3-(1-ethyl-4-{[4-(pyrrolidin-1-yl)piperidin-1-yl]methyl}-1H-indol-2- |
| | yl)prop-2-yn-1-yl]pyridin-3-amine |
| 82. | 3-(1-ethyl-5-{[(1-methylpiperidin-4-yl)amino]methyl}-1H-indol-2-yl)prop-2-yn-1-yl |
| | benzoate |
| 83. | 2-[5-({3-[1-(2-chloroethyl)-5-{[(1-methylpiperidin-4-yl)amino]methyl}-1H-indol-2- |
| | yl]prop-2-yn-1-yl}amino)pyridin-2-yl]-2-methylpropanenitrile |
| 84. | N-(6-chloropyridin-3-yl)-3-(1-ethyl-5-{[(1-methylpiperidin-4-yl)amino]methyl}-1H- |
| | indol-2-yl)prop-2-ynamide |
| 85. | N-[6-(1-cyano-1-methylethyl)pyridin-3-yl]-3-(1-ethyl-5-{[(1-methylpiperidin-4- |
| | yl)amino]methyl}-1H-indol-2-yl)prop-2-ynamide |
| 86. | N-({3-ethyl-2-[3-(phenylamino)prop-1-yn-1-yl]-1H-indol-6-yl}methyl)oxan-4-amine |
| 87. | 2-[5-({3-[1-(2-chloroethyl)-4-{[4-(pyrrolidin-1-yl)piperidin-1-yl]methyl}-1H-indol-2- |
| | yl]prop-2-yn-1-yl}amino)pyridin-2-yl]-2-methylpropanenitrile |
| 88. | 2-(5-{[3-(5-{[(1-methanesulfonylpiperidin-4-yl)amino]methyl}-1-(2,2,2-trifluoroethyl)- |
| | 1H-indol-2-yl)prop-2-yn-1-yl]amino}pyridin-2-yl)-2-methylpropanenitrile |
| 89. | 2-[5-({3-[5-({[1-(2-methanesulfonylethyl)piperidin-4-yl]amino}methyl)-1-(2,2,2- |
| | trifluoroethyl)-1H-indol-2-yl]prop-2-yn-1-yl}amino)pyridin-2-yl]-2-methylpropanenitrile |
| 90. | 2-(5-{[3-(5-{[(1-acetylpiperidin-4-yl)amino]methyl}-1-(2,2,2-trifluoroethyl)-1H-indol-2- |
| | yl)prop-2-yn-1-yl]amino}pyridin-2-yl)-2-methylpropanenitrile |
| 91. | 2-{5-[(3-{5-[({1-[2-(dimethylamino)acetyl]piperidin-4-yl}amino)methyl]-1-(2,2,2- |
| | trifluoroethyl)-1H-indol-2-yl}prop-2-yn-1-yl)amino]pyridin-2-yl}-2-methylpropanenitrile |
| 92. | 2-methyl-2-(5-{[3-(5-{[(1-methylpiperidin-4-yl)amino]methyl}-1-(2,2,2-trifluoroethyl)- |
| | 1H-indol-2-yl)prop-2-yn-1-yl]amino}pyridin-2-yl)propanenitrile |
| 93. | 2-methyl-2-{5-[(3-{5-[(methylamino)methyl]-1-(2,2,2-trifluoroethyl)-1H-indol-2- |
| | yl}prop-2-yn-1-yl)amino]pyridin-2-yl}propanenitrile |
| 94. | 6-Chloro-N-[3-(5-{[(1-methylpiperidin-4-yl)amino]methyl}-1-(2,2,2-trifluoroethyl)-1H- |
| | indol-2-yl)prop-2-yn-1-yl]pyridin-3-amine |
| 95. | 6-chloro-N-[3-(5-{[(oxan-4-yl)amino]methyl}-1-(2,2,2-trifluoroethyl)-1H-indol-2- |
| | yl)prop-2-yn-1-yl]pyridin-3-amine |
| 96. | 2-[5-({3-[1-(cyclopropylmethyl)-5-{[(oxan-4-yl)amino]methyl}-1H-indol-2-yl]prop-2-yn- |
| | 1-yl}amino)pyridin-2-yl]-2-methylpropanenitrile |
| 97. | 2-(5-{[3-(4-{[4-(diethylamino)piperidin-1-yl]methyl}-1-(2,2,2-trifluoroethyl)-1H-indol-2- |
| | yl)prop-2-yn-1-yl]amino}pyridin-2-yl)-2-methylpropanenitrile |
| 98. | 2-methyl-2-{5-[(3-{4-[(4-methylpiperazin-1-yl)methyl]-1-(2,2,2-trifluoroethyl)-1H-indol- |
| | 2-yl}prop-2-yn-1-yl)amino]pyridin-2-yl}propanenitrile |
| 99. | 2-(5-{[3-(1-ethyl-7-fluoro-4-{[4-(pyrrolidin-1-yl)piperidin-1-yl]methyl}-1H-indol-2- |
| | yl)prop-2-yn-1-yl]amino}pyridin-2-yl)-2-methylpropanenitrile |
| 100. | 2-methyl-2-(5-{[3-(4-{[4-(pyrrolidin-1-yl)piperidin-1-yl]methyl}-1-(2,2,2-trifluoroethyl)- |
| | 1H-indol-2-yl)prop-2-yn-1-yl]amino}pyridin-2-yl)propanenitrile |
| 101. | 2-(5-{[3-(4-{[4-(4-hydroxypiperidin-1-yl)piperidin-1-yl]methyl}-1-(2,2,2-trifluoroethyl)- |
| | 1H-indol-2-yl)prop-2-yn-1-yl]amino}pyridin-2-yl)-2-methylpropanenitrile |
| 102. | N-(6-cyanopyridin-3-yl)-3-(1-ethyl-5-{[(1-methylpiperidin-4-yl)amino]methyl}-1H- |
| | indol-2-yl)prop-2-ynamide |
| 103. | N-[6-(1-cyano-1-methylethyl)pyridin-3-yl]-3-(5-{[(oxan-4-yl)amino]methyl}-1-(2,2,2- |
| | trifluoroethyl)-1H-indol-2-yl)prop-2-ynamide |
| 104. | N-[6-(1-cyano-1-methylethyl)pyridin-3-yl]-3-(5-{[(1-methylpiperidin-4- |
| | yl)amino]methyl}-1-(2,2,2-trifluoroethyl)-1H-indol-2-yl)prop-2-ynamide |
| 105. | 2-methyl-2-(5-{[3-(5-{[(oxan-4-yl)amino]methyl}-1-(oxiran-2-ylmethyl)-1H-indol-2- |
| | yl)prop-2-yn-1-yl]amino}pyridin-2-yl)propanenitrile |
| 106. | 2-(5-{[3-(5-{[(2-methoxyethyl)amino]methyl}-1-(2,2,2-trifluoroethyl)-1H-indol-2- |
| | yl)prop-2-yn-1-yl]amino}pyridin-2-yl)-2-methylpropanenitrile |
| 107. | 2-methyl-2-[5-({3-[5-({[2-(morpholin-4-yl)ethyl]amino}methyl)-1-(2,2,2-trifluoroethyl)- |
| | 1H-indol-2-yl]prop-2-yn-1-yl}amino)pyridin-2-yl]propanenitrile |
| 108. | 2-methyl-2-(5-{[3-(4-{[(1-methylpiperidin-4-yl)amino]methyl}-1-(2,2,2-trifluoroethyl)- |
| | 1H-indol-2-yl)prop-2-yn-1-yl]amino}pyridin-2-yl)propanenitrile |
| 109. | 2-methyl-2-(5-{[3-(4-{[(oxan-4-yl)amino]methyl}-1-(2,2,2-trifluoroethyl)-1H-indol-2- |
| | yl)prop-2-yn-1-yl]amino}pyridin-2-yl)propanenitrile |
| 110. | 2-[5-({3-[5-({[2-(dimethylamino)ethyl]amino}methyl)-1-(2,2,2-trifluoroethyl)-1H-indol- |
| | 2-yl]prop-2-yn-1-yl}amino)pyridin-2-yl]-2-methylpropanenitrile |
| 111. | 2-(5-{[3-(7-fluoro-4-{[4-(pyrrolidin-1-yl)piperidin-1-yl]methyl}-1-(2,2,2-trifluoroethyl)- |
| | 1H-indol-2-yl)prop-2-yn-1-yl]amino}pyridin-2-yl)-2-methylpropanenitrile |
| 112. | 2-methyl-2-[5-({3-[1-(2,2,2-trifluoroethyl)-5-{[(2,2,2-trifluoroethyl)amino]methyl}-1H- |
| | indol-2-yl]prop-2-yn-1-yl}amino)pyridin-2-yl]propanenitrile |
| 113. | 2-[5-({3-[5-({[1-(2-hydroxyethyl)piperidin-4-yl]amino}methyl)-1-(2,2,2-trifluoroethyl)- |
| | 1H-indol-2-yl]prop-2-yn-1-yl}amino)pyridin-2-yl]-2-methylpropanenitrile |
| 114. | 2-[5-({3-[5-({[1-(2-methoxyethyl)piperidin-4-yl]amino}methyl)-1-(2,2,2-trifluoroethyl)- |
| | 1H-indol-2-yl]prop-2-yn-1-yl}amino)pyridin-2-yl]-2-methylpropanenitrile |
| 115. | 2-[5-({3-[5-({[4-(dimethylamino)cyclohexyl]amino}methyl)-1-(2,2,2-trifluoroethyl)-1H- |
| | indol-2-yl]prop-2-yn-1-yl}amino)pyridin-2-yl]-2-methylpropanenitrile |
| 116. | 2-methyl-2-{5-[(3-{5-[({1-[2-(morpholin-4-yl)acetyl]piperidin-4-yl}amino)methyl]-1- |
| | (2,2,2-trifluoroethyl)-1H-indol-2-yl}prop-2-yn-1-yl)amino]pyridin-2-yl}propanenitrile |
| 117. | 2-(5-{[3-(4-{[(2-methoxyethyl)amino]methyl}-1-(2,2,2-trifluoroethyl)-1H-indol-2- |
| | yl)prop-2-yn-1-yl]amino}pyridin-2-yl)-2-methylpropanenitrile |
| 118. | 2-methyl-2-{5-[(3-{4-[(methylamino)methyl]-1-(2,2,2-trifluoroethyl)-1H-indol-2- |
| | yl}prop-2-yn-1-yl)amino]pyridin-2-yl}propanenitrile |
| 119. | 2-{5-[(3-{4-[(4-acetylpiperazin-1-yl)methyl]-1-(2,2,2-trifluoroethyl)-1H-indol-2-yl}prop- |
| | 2-yn-1-yl)amino]pyridin-2-yl}-2-methylpropanenitrile |
| 120. | 2-methyl-2-[5-({3-[4-(morpholin-4-ylmethyl)-1-(2,2,2-trifluoroethyl)-1H-indol-2-yl]prop- |
| | 2-yn-1-yl}amino)pyridin-2-yl]propanenitrile |
| 121. | 2-(5-{[3-(4-{[4-(dimethylamino)piperidin-1-yl]methyl}-1-(2,2,2-trifluoroethyl)-1H-indol- |
| | 2-yl)prop-2-yn-1-yl]amino}pyridin-2-yl)-2-methylpropanenitrile |
| 122. | 2-[5-({3-[4-(hydroxymethyl)-1-(2,2,2-trifluoroethyl)-1H-indol-2-yl]prop-2-yn-1- |
| | yl}amino)pyridin-2-yl]-2-methylpropanenitrile |
| 123. | 2-methyl-2-[5-({3-[4-({4-[2-(morpholin-4-yl)-2-oxoethyl]piperazin-1-yl}methyl)-1- |
| | (2,2,2-trifluoroethyl)-1H-indol-2-yl]prop-2-yn-1-yl}amino)pyridin-2-yl]propanenitrile |
| 124. | 2-(5-{[3-(3-ethyl-7-{[4-(pyrrolidin-1-yl)piperidin-1-yl]methyl}-1H-indol-2-yl)prop-2-yn- |
| | 1-yl]amino}pyridin-2-yl)-2-methylpropanenitrile |
| 125. | methyl 5-{[3-(5-{[(oxan-4-yl)amino]methyl}-1-(2,2,2-trifluoroethyl)-1H-indol-2-yl)prop- |
| | 2-yn-1-yl]amino}pyridine-2-carboxylate |
| 126. | N-methyl-5-{[3-(5-{[(oxan-4-yl)amino]methyl}-1-(2,2,2-trifluoroethyl)-1H-indol-2- |
| | yl)prop-2-yn-1-yl]amino}pyridine-2-carboxamide |
| 127. | N-(2-hydroxyethyl)-5-{[3-(5-{[(oxan-4-yl)amino]methyl}-1-(2,2,2-trifluoroethyl)-1H- |
| | indol-2-yl)prop-2-yn-1-yl]amino}pyridine-2-carboxamide |
| 128. | N-(2-methoxyethyl)-5-{[3-(5-{[(oxan-4-yl)amino]methyl}-1-(2,2,2-trifluoroethyl)-1H- |
| | indol-2-yl)prop-2-yn-1-yl]amino}pyridine-2-carboxamide |
| 129. | 2-[(5-{[3-(5-{[(oxan-4-yl)amino]methyl}-1-(2,2,2-trifluoroethyl)-1H-indol-2-yl)prop-2- |
| | yn-1-yl]amino}pyridin-2-yl)formamido]acetic acid |
| 130. | 5-{[3-(5-{[(oxan-4-yl)amino]methyl}-1-(2,2,2-trifluoroethyl)-1H-indol-2-yl)prop-2-yn-1- |
| | yl]amino}pyridine-2-carboxylic acid |
| 131. | N-(2-methanesulfonylethyl)-5-{[3-(5-{[(oxan-4-yl)amino]methyl}-1-(2,2,2- |
| | trifluoroethyl)-1H-indol-2-yl)prop-2-yn-1-yl]amino}pyridine-2-carboxamide |
| 132. | 2-[5-({3-[1-(cyanomethyl)-5-{[(oxan-4-yl)amino]methyl}-1H-indol-2-yl]prop-2-yn-1- |
| | yl}amino)pyridin-2-yl]-2-methylpropanenitrile |
| 133. | 2-methyl-2-[5-({3-[1-(2-methylpropyl)-5-{[(oxan-4-yl)amino]methyl}-1H-indol-2- |
| | yl]prop-2-yn-1-yl}amino)pyridin-2-yl]propanenitrile |
| 134. | 2-methyl-2-{5-[(3-{4-[(oxan-4-yl)amino]-1-(2,2,2-trifluoroethyl)-1H-indol-2-yl}prop-2- |
| | yn-1-yl)amino]pyridin-2-yl}propanenitrile |
| 135. | 5-{[3-(5-{[(oxan-4-yl)amino]methyl}-1-(2,2,2-trifluoroethyl)-1H-indol-2-yl)prop-2-yn-1- |
| | yl]amino}pyridine-2-carbonitrile |
| 136. | N,N-dimethyl-5-{[3-(5-{[(oxan-4-yl)amino]methyl}-1-(2,2,2-trifluoroethyl)-1H-indol-2- |
| | yl)prop-2-yn-1-yl]amino}pyridine-2-carboxamide |
| 137. | N-(oxan-4-yl)-5-{[3-(5-{[(oxan-4-yl)amino]methyl}-1-(2,2,2-trifluoroethyl)-1H-indol-2- |
| | yl)prop-2-yn-1-yl]amino}pyridine-2-carboxamide |
| 138. | 2-tert-butyl-N-[3-(5-{[(oxan-4-yl)amino]methyl}-1-(2,2,2-trifluoroethyl)-1H-indol-2- |
| | yl)prop-2-yn-1-yl]pyrimidin-5-amine |
| 139. | N-(1-methylpiperidin-4-yl)-5-{[3-(5-{[(oxan-4-yl)amino]methyl}-1-(2,2,2-trifluoroethyl)- |
| | 1H-indol-2-yl)prop-2-yn-1-yl]amino}pyridine-2-carboxamide |
| 140. | N-[6-(1-cyano-1-methylethyl)pyridin-3-yl]-3-[1-(2-fluoroethyl)-5-{[(oxan-4- |
| | yl)amino]methyl}-1H-indol-2-yl]prop-2-ynamide |
| 141. | 2-(5-{[3-(7-chloro-4-{[4-(pyrrolidin-1-yl)piperidin-1-yl]methyl}-1-(2,2,2-trifluoroethyl)- |
| | 1H-indol-2-yl)prop-2-yn-1-yl]amino}pyridin-2-yl)-2-methylpropanenitrile |
| 142. | 2-(5-{[3-(6-fluoro-4-{[4-(pyrrolidin-1-yl)piperidin-1-yl]methyl}-1-(2,2,2-trifluoroethyl)- |
| | 1H-indol-2-yl)prop-2-yn-1-yl]amino}pyridin-2-yl)-2-methylpropanenitrile |
| 143. | 2-(5-{[3-(1-ethyl-5-{[(oxan-4-yl)amino]methyl}-1H-pyrrolo[2,3-c]pyridin-2-yl)prop-2- |
| | yn-1-yl]amino}pyridin-2-yl)-2-methylpropanenitrile |
| 144. | 2-methyl-2-(5-{[3-(5-{[(oxan-4-yl)amino]methyl}-1-(2,2,2-trifluoroethyl)-1H- |
| | pyrrolo[2,3-c]pyridin-2-yl)prop-2-yn-1-yl]amino}pyridin-2-yl)propanenitrile |
| 145. | 2-(5-{[3-(5-{[4-(dimethylamino)piperidin-1-yl]methyl}-1-(2,2,2-trifluoroethyl)-1H- |
| | pyrrolo[2,3-c]pyridin-2-yl)prop-2-yn-1-yl]amino}pyridin-2-yl)-2-methylpropanenitrile |
| 146. | 2-(5-{[3-(7-chloro-4-{[4-(pyrrolidin-1-yl)piperidin-1-yl]methyl}-1-(2,2,2-trifluoroethyl)- |
| | 1H-pyrrolo[2,3-c]pyridin-2-yl)prop-2-yn-1-yl]amino}pyridin-2-yl)-2- |
| | methylpropanenitrile |
| 147. | 2-(5-{[3-(4-{[4-(dimethylamino)-piperidin-1-yl]methyl}-6-fluoro-1-(2,2,2-trifluoroethyl)- |
| | 1H-indol-2-yl)prop-2-yn-1-yl]amino}pyridin-2-yl)-2-methylpropanenitrile |
| 148. | 2-(5-{[3-(4-{[4-(diethylamino)piperidin-1-yl]methyl}-6-fluoro-1-(2,2,2-trifluoroethyl)- |
| | 1H-indol-2-yl)prop-2-yn-1-yl]amino}pyridin-2-yl)-2-methylpropanenitrile |
| 149. | 2-(5-{[3-(6-fluoro-4-{[4-(4-hydroxypiperidin-1-yl)piperidin-1-yl]methyl}-1-(2,2,2- |
| | trifluoroethyl)-1H-indol-2-yl)prop-2-yn-1-yl]amino}pyridin-2-yl)-2-methylpropanenitrile |
| 150. | 2-(5-{[3-(6-fluoro-5-{[(oxan-4-yl)amino]methyl}-1-(2,2,2-trifluoroethyl)-1H-indol-2- |
| | yl)prop-2-yn-1-yl]amino}pyridin-2-yl)-2-methylpropanenitrile |
| 151. | 2-(5-{[3-(5-{[(1-acetylpiperidin-4-yl)amino]methyl}-6-fluoro-1-(2,2,2-trifluoroethyl)-1H- |
| | indol-2-yl)prop-2-yn-1-yl]amino}pyridin-2-yl)-2-methylpropanenitrile |
| 152. | 2-(5-{[3-(6-chloro-4-{[4-(dimethylamino)piperidin-1-yl]methyl}-1-(2,2,2-trifluoroethyl)- |
| | 1H-indol-2-yl)prop-2-yn-1-yl]amino}pyridin-2-yl)-2-methylpropanenitrile |
| 153. | 2-(5-{[3-(6-chloro-4-{[4-(diethylamino)piperidin-1-yl]methyl}-1-(2,2,2-trifluoroethyl)- |
| | 1H-indol-2-yl)prop-2-yn-1-yl]amino}pyridin-2-yl)-2-methylpropanenitrile |
| 154. | 2-(5-{[3-(6-chloro-4-{[4-(4-hydroxypiperidin-1-yl)piperidin-1-yl]methyl}-1-(2,2,2- |
| | trifluoroethyl)-1H-indol-2-yl)prop-2-yn-1-yl]amino}pyridin-2-yl)-2-methylpropanenitrile |
| 155. | 2-(5-{[3-(4-{[4-(2-methanesulfonyl-ethyl)piperazin-1-yl]methyl}-1-(2,2,2-trifluoroethyl)- |
| | 1H-indol-2-yl)prop-2-yn-1-yl]amino}pyridin-2-yl)-2-methylpropanenitrile |
| 156. | 2-(4-{[2-(3-{[6-(1-cyano-1-methylethyl)pyridin-3-yl]amino}prop-1-yn-1-yl)-1-(2,2,2- |
| | trifluoroethyl)-1H-indol-4-yl]methyl}piperazin-1-yl)-N,N-dimethylacetamide |
| 157. | 2-methyl-2-{5-[(3-{4-[(3-oxopiperazin-1-yl]methyl]-1-(2,2,2-trifluoroethyl)-1H-indol-2- |
| | yl}prop-2-yn-1-yl)amino]pyridin-2-yl}propanenitrile |
| 158. | 2-methyl-2-[5-({3-[4-({4-[2-(morpholin-4-yl)-2-oxoethyl]piperidin-1-yl}methyl)-1- |
| | (2,2,2-trifluoroethyl)-1H-indol-2-yl]prop-2-yn-1-yl}amino)pyridin-2-yl]propanenitrile |
| 159. | 2-(4-{[2-(3-{[6-(1-cyano-1-methylethyl)pyridin-3-yl]amino}prop-1-yn-1-yl)-1-(2,2,2- |
| | trifluoroethyl)-1H-indol-4-yl]methyl}piperazin-1-yl)acetamide |
| 160. | N-[6-(1-cyano-1-methylethyl)pyridin-3-yl]-3-(4-{[4-(pyrrolidin-1-yl)piperidin-1- |
| | yl]methyl}-1-(2,2,2-trifluoroethyl)-1H-indol-2-yl)prop-2-ynamide |
| 161. | 2-(1-{[2-(3-{[6-(1-cyano-1-methylethyl)pyridin-3-yl]amino}prop-1-yn-1-yl)-1-(2,2,2- |
| | trifluoroethyl)-1H-indol-4-yl]methyl}piperidin-4-yl)acetamide |
| 162. | 2-(5-{[3-(4-{[4-(2-aminoethyl)-piperazin-1-yl]methyl}-1-(2,2,2-trifluoroethyl)-1H-indol- |
| | 2-yl)prop-2-yn-1-yl]amino}pyridin-2-yl)-2-methylpropanenitrile |
| 163. | 2-(1-{[2-(3-{[6-(1-cyano-1-methylethyl)pyridin-3-yl]amino}prop-1-yn-1-yl)-1-(2,2,2- |
| | trifluoroethyl)-1H-indol-4-yl]methyl}piperidin-4-yl)-N,N-dimethylacetamide |
| 164. | 2-methyl-2-(5-{[3-(4-{[4-(morpholin-4-yl)piperidin-1-yl]methyl}-1-(2,2,2-trifluoroethyl)- |
| | 1H-indol-2-yl)prop-2-yn-1-yl]amino}pyridin-2-yl)propanenitrile |
| 165. | 2-(5-{[3-(4-{[4-(4-aminopiperidin-1-yl)piperidin-1-yl]methyl}-1-(2,2,2-trifluoroethyl)- |
| | 1H-indol-2-yl)prop-2-yn-1-yl]amino}pyridin-2-yl)-2-methylpropanenitrile |
| 166. | 2-methyl-2-[5-({3-[1-(oxiran-2-ylmethyl)-4-{[4-(pyrrolidin-1-yl)piperidin-1-yl]methyl}- |
| | 1H-indol-2-yl]prop-2-yn-1-yl}amino)pyridin-2-yl]propanenitrile |
| 167. | 2-(5-{[3-(3-ethyl-6-{[(oxan-4-yl)amino]methyl}-1H-indol-2-yl)prop-2-yn-1- |
| | yl]amino}pyridin-2-yl)-2-methylpropanenitrile |
| 168. | 2-methyl-2-(5-{[3-(6-{[(oxan-4-yl)amino]methyl}-3-(2,2,2-trifluoroethyl)-1H-indol-2- |
| | yl)prop-2-yn-1-yl]amino}pyridin-2-yl)propanenitrile |
| 169. | 2-(5-{[3-(1-acetyl-3-ethyl-6-{[(oxan-4-yl)amino]methyl}-1H-indol-2-yl)prop-2-yn-1- |
| | yl]amino}pyridin-2-yl)-2-methylpropanenitrile |
| 170. | 2-(5-{[3-(3-ethyl-6-{[(1-methylpiperidin-4-yl)amino]methyl}-1H-indol-2-yl)prop-2-yn-1- |
| | yl]amino}pyridin-2-yl)-2-methylpropanenitrile |
| 171. | 2-methyl-2-(5-{[3-(6-{[(1-methylpiperidin-4-yl)amino]methyl}-3-(2,2,2-trifluoroethyl)- |
| | 1H-indol-2-yl)prop-2-yn-1-yl]amino}pyridin-2-yl)propanenitrile |
| 172. | 2-{5-[(3-{6-chloro-4-[(1-methylpiperidin-4-yl)amino]-1-(2,2,2-trifluoroethyl)-1H-indol- |
| | 2-yl}prop-2-yn-1-yl)amino]pyridin-2-yl}-2-methylpropanenitrile |
| 173. | 2-(3-{[6-(1-cyano-1-methylethyl)pyridin-3-yl]amino}prop-1-yn-1-yl)-6-fluoro-N-(1- |
| | methylpiperidin-4-yl)-1-(2,2,2-trifluoroethyl)-1H-indole-4-carboxamide |
| 174. | 2-[5-({3-[6-fluoro-4-(4-methylpiperazine-1-carbonyl)-1-(2,2,2-trifluoroethyl)-1H-indol-2- |
| | yl]prop-2-yn-1-yl}amino)pyridin-2-yl]-2-methylpropanenitrile |
| 175. | 6-fluoro-2-{3-[(6-methanesulfonylpyridin-3-yl)amino]prop-1-yn-1-yl}-N-(1- |
| | methylpiperidin-4-yl)-1-(2,2,2-trifluoroethyl)-1H-indol-4-amine |
| 176. | 2-{5-[(3-{6-fluoro-4-[(1-methylpiperidin-4-yl)amino]-1-(2,2,2-trifluoroethyl)-1H-indol-2- |
| | yl}prop-2-yn-1-yl)amino]pyridin-2-yl}-2-methylpropanenitrile |
| 177. | 5-[(3-{6-fluoro-4-[(1-methylpiperidin-4-yl)amino]-1-(2,2,2-trifluoroethyl)-1H-indol-2- |
| | yl}prop-2-yn-1-yl)amino]-N-(pyridin-3-yl)pyridine-2-carboxamide |
| 178. | 2-methyl-2-(5-{[3-(5-{[(oxan-4-yl)amino]methyl}-1-(2,2,2-trifluoroethyl)-1H- |
| | pyrrolo[2,3-b]pyridin-2-yl)prop-2-yn-1-yl]amino}pyridin-2-yl)propanenitrile |
| 179. | 2-methyl-2-{5-[(3-{4-[(1-methylpiperidin-4-yl)amino]-1-(2,2,2-trifluoroethyl)-1H- |
| | pyrrolo[2,3-b]pyridin-2-yl}prop-2-yn-1-yl)amino]pyridin-2-yl}propanenitrile |
| 180. | 2-(5-{[3-(7-chloro-1-ethyl-4-{[4-(pyrrolidin-1-yl)piperidin-1-yl]methyl}-1H-indol-2- |
| | yl)prop-2-yn-1-yl]amino}pyridin-2-yl)-2-methylpropanenitrile |
| 181. | 2-(5-{[3-(7-chloro-5-{[(oxan-4-yl)amino]methyl}-1-(2,2,2-trifluoroethyl)-1H-indol-2- |
| | yl)prop-2-yn-1-yl]amino}pyridin-2-yl)-2-methylpropanenitrile |
| 182. | 2-(5-{[3-(7-chloro-5-{[(1-methylpiperidin-4-yl)amino]methyl}-1-(2,2,2-trifluoroethyl)- |
| | 1H-indol-2-yl)prop-2-yn-1-yl]amino}pyridin-2-yl)-2-methylpropanenitrile |
| 183. | 2-{5-[(3-{7-fluoro-4-[(1-methylpiperidin-4-yl)amino]-1-(2,2,2-trifluoroethyl)-1H-indol-2- |
| | yl}prop-2-yn-1-yl)amino]pyridin-2-yl}-2-methylpropanenitrile |
| 184. | 2-(5-{[3-(7-fluoro-5-{[(oxan-4-yl)amino]methyl}-1-(2,2,2-trifluoroethyl)-1H-indol-2- |
| | yl)prop-2-yn-1-yl]amino}pyridin-2-yl)-2-methylpropanenitrile |
| 185. | 2-(5-{[3-(7-fluoro-5-{[(1-methylpiperidin-4-yl)amino]methyl}-1-(2,2,2-trifluoroethyl)- |
| | 1H-indol-2-yl)prop-2-yn-1-yl]amino}pyridin-2-yl)-2-methylpropanenitrile |
| 186. | 2-methyl-2-(5-{[3-(5-{[(oxan-4-yl)amino]methyl}-1-(2,2,2-trifluoroethyl)-1H-1,3- |
| | benzodiazol-2-yl)prop-2-yn-1-yl]amino}pyridin-2-yl)propanenitrile |
| 187. | N-{[2-(2-phenylethynyl)-1-(2,2,2-trifluoroethyl)-1H-1,3-benzodiazol-5-yl]methyl}oxan- |
| | 4-amine |
| 188. | 2-methyl-2-(5-{[3-(5-{[(oxan-4-yl)amino]methyl}-1-(2,2,2-trifluoroethyl)-1H- |
| | pyrrolo[3,2-b]pyridin-2-yl)prop-2-yn-1-yl]amino}pyridin-2-yl)propanenitrile |
| 189. | 2-{3-[(4-methanesulfonylphenyl)amino]prop-1-yn-1-yl}-5-methyl-N-(1-methylpiperidin- |
| | 4-yl)-1-(2,2,2-trifluoroethyl)-1H-indol-4-amine |
| 190. | 4-[(2-{3-[(4-methanesulfonylphenyl)amino]prop-1-yn-1-yl}-5-methyl-1-(2,2,2- |
| | trifluoroethyl)-1H-indol-4-yl)amino]-1λ⁶-thiane-1,1-dione |
| 191. | 2-methyl-2-{5-[(3-{5-methyl-4-[(1-methylpiperidin-4-yl)amino]-1-(2,2,2-trifluoroethyl)- |
| | 1H-indol-2-yl}prop-2-yn-1-yl)amino]pyridin-2-yl}propanenitrile |
| 192. | N-[1-(2-methanesulfonylethyl)piperidin-4-yl]-2-{3-[(4- |
| | methanesulfonylphenyl)amino]prop-1-yn-1-yl}-5-methyl-1-(2,2,2-trifluoroethyl)-1H- |
| | indol-4-amine |
| 193. | 4-[(3-{5-methyl-4-[(piperidin-4-yl)amino]-1-(2,2,2-trifluoroethyl)-1H-indol-2-yl}prop-2- |
| | yn-1-yl)amino]benzene-1-sulfonamide |
| 194. | 2-{3-[(4-methanesulfonylphenyl)amino]prop-1-yn-1-yl}-5-methyl-N-[1-(oxan-4- |
| | yl)piperidin-4-yl]-1-(2,2,2-trifluoroethyl)-1H-indol-4-amine |
| 195. | 2-{4-[(2-{3-[(4-methanesulfonylphenyl)amino]prop-1-yn-1-yl}-5-methyl-1-(2,2,2- |
| | trifluoroethyl)-1H-indol-4-yl)amino]piperidin-1-yl}ethan-1-ol |
| 196. | 2-[5-({3-[4-(methoxymethyl)-1-(2,2,2-trifluoroethyl)-1H-indol-2-yl]prop-2-yn-1- |
| | yl}amino)pyridin-2-yl]-2-methylpropanenitrile |
| 197. | 2-[5-({3-[4-(cyanomethyl)-1-(2,2,2-trifluoroethyl)-1H-indol-2-yl]prop-2-yn-1- |
| | yl}amino)pyridin-2-yl]-2-methylpropanenitrile |
| 198. | 2-methyl-2-[5-({3-[5-(morpholine-4-carbonyl)-1-(2,2,2-trifluoroethyl)-1H-indol-2- |
| | yl]prop-2-yn-1-yl}amino)pyridin-2-yl]propanenitrile |
| 199. | 2-methyl-2-[5-({3-[5-(4-methylpiperazine-1-carbonyl)-1-(2,2,2-trifluoroethyl)-1H-indol- |
| | 2-yl]prop-2-yn-1-yl}amino)pyridin-2-yl]propanenitrile |
| 200. | 2-{5-[(3-{5-[4-(dimethylamino)piperidine-1-carbonyl]-1-(2,2,2-trifluoroethyl)-1H-indol- |
| | 2-yl}prop-2-yn-1-yl)amino]pyridin-2-yl}-2-methylpropanenitrile |
| 201. | 2-(3-{[6-(1-cyano-1-methylethyl)pyridin-3-yl]amino}prop-1-yn-1-yl)-N-{1-[2- |
| | (dimethylamino)acetyl]piperidin-4-yl}-1-(2,2,2-trifluoroethyl)-1H-indole-5-carboxamide |
| 202. | 2-(3-{[6-(1-cyano-1-methylethyl)pyridin-3-yl]amino}prop-1-yn-1-yl)-N-(oxan-4-yl)-1- |
| | (2,2,2-trifluoroethyl)-1H-indole-5-carboxamide |
| 203. | 2-methyl-2-(5-{[3-(5-{1-[(oxan-4-yl)amino]ethyl}-1-(2,2,2-trifluoroethyl)-1H-indol-2- |
| | yl)prop-2-yn-1-yl]amino}pyridin-2-yl)propanenitrile |
| 204. | 2-methyl-2-{5-[(3-{4-[(1-methylpiperidin-4-yl)amino]-1-(2,2,2-trifluoroethyl)-1H- |
| | pyrrolo[3,2-c]pyridin-2-yl}prop-2-yn-1-yl)amino]pyridin-2-yl}propanenitrile |
| 205. | 2-methyl-2-[5-({3-[5-(morpholin-4-ylmethyl)-1-(2,2,2-trifluoroethyl)-1H-indol-2-yl]prop- |
| | 2-yn-1-yl}amino)pyridin-2-yl]propanenitrile |
| 206. | 2-[5-({3-[5-({[1-(2-cyanoethyl)-piperidin-4-yl]amino}methyl)-1-(2,2,2-trifluoroethyl)- |
| | 1H-indol-2-yl]prop-2-yn-1-yl}amino)pyridin-2-yl]-2-methylpropanenitrile |
| 207. | 2-methyl-2-(5-{[3-(5-{[(1-methylazetidin-3-yl)amino]methyl}-1-(2,2,2-trifluoroethyl)- |
| | 1H-indol-2-yl)prop-2-yn-1-yl]amino}pyridin-2-yl)propanenitrile |
| 208. | 2-methyl-2-(5-{[3-(5-{[(oxetan-3-yl)amino]methyl}-1-(2,2,2-trifluoroethyl)-1H-indol-2- |
| | yl)prop-2-yn-1-yl]amino}pyridin-2-yl)propanenitrile |
| 209. | 2-(5-{[3-(5-{[4-(dimethylamino)-piperidin-1-yl]methyl}-1-(2,2,2-trifluoroethyl)-1H- |
| | indol-2-yl)prop-2-yn-1-yl]amino}pyridin-2-yl)-2-methylpropanenitrile |
| 210. | 2-methyl-2-{5-[(3-{5-[({1-[2-(4-methylpiperazin-1-yl)acetyl]piperidin-4- |
| | yl}amino)methyl]-1-(2,2,2-trifluoroethyl)-1H-indol-2-yl}prop-2-yn-1-yl)amino]pyridin- |
| | 2-yl}propanenitrile |
| 211. | 2-(5-{[3-(5-{[(1-methoxypropan-2-yl)amino]methyl}-1-(2,2,2-trifluoroethyl)-1H-indol-2- |
| | yl)prop-2-yn-1-yl]amino}pyridin-2-yl)-2-methylpropanenitrile |
| 212. | 2-methyl-2-(5-{[3-(5-{[(pyridin-4-ylmethyl)amino]methyl}-1-(2,2,2-trifluoroethyl)-1H- |
| | indol-2-yl)prop-2-yn-1-yl]amino}pyridin-2-yl)propanenitrile |
| 213. | 2-methyl-2-(5-{[3-(5-{[(pyridin-3-ylmethyl)amino]methyl}-1-(2,2,2-trifluoroethyl)-1H- |
| | indol-2-yl)prop-2-yn-1-yl]amino}pyridin-2-yl)propanenitrile |
| 214. | 2-[5-({3-[5-({[1-(dimethylamino)-propan-2-yl]amino}methyl)-1-(2,2,2-trifluoroethyl)- |
| | 1H-indol-2-yl]prop-2-yn-1-yl}amino)pyridin-2-yl]-2-methylpropanenitrile |
| 215. | 2-[4-({[2-(3-{[6-(1-cyano-1-methylethyl)pyridin-3-yl]amino}prop-1-yn-1-yl)-1-(2,2,2- |
| | trifluoroethyl)-1H-indol-5-yl]methyl}amino)piperidin-1-yl]-N-(oxan-4-yl)acetamide |
| 216. | 2-[5-({3-[5-({[1-(2-methoxyacetyl)-piperidin-4-yl]amino}methyl)-1-(2,2,2- |
| | trifluoroethyl)-1H-indol-2-yl]prop-2-yn-1-yl}amino)pyridin-2-yl]-2-methylpropanenitrile |
| 217. | 2-methyl-2-{5-[(3-{5-[({1-[2-(oxan-4-yl)acetyl]piperidin-4-yl}amino)-methyl]-1-(2,2,2- |
| | trifluoroethyl)-1H-indol-2-yl}prop-2-yn-1-yl)amino]-pyridin-2-yl}propanenitrile |
| 218. | 2-methyl-2-{5-[(3-{5-[({1-[2-(pyridin-3-yl)acetyl]piperidin-4-yl}amino)-methyl]-1- |
| | (2,2,2-trifluoroethyl)-1H-indol-2-yl}prop-2-yn-1-yl)amino]-pyridin-2-yl}propanenitrile |
| 219. | 2-methyl-2-(5-{[3-(5-{[(1-{2-[(oxan-4-yl)amino]acetyl}piperidin-4-yl)amino]methyl}-1- |
| | (2,2,2-trifluoroethyl)-1H-indol-2-yl)prop-2-yn-1-yl]amino}pyridin-2-yl)-propanenitrile |
| 220. | 2-[4-({[2-(3-{[6-(1-cyano-1-methylethyl)pyridin-3-yl]amino}prop-1-yn-1-yl)-1-(2,2,2- |
| | trifluoroethyl)-1H-indol-5-yl]methyl}amino)piperidin-1-yl]-N-methyl-N-(propan-2- |
| | yl)acetamide |
| 221. | 2-[4-({[2-(3-{[6-(1-cyano-1-methylethyl)pyridin-3-yl]amino}prop-1-yn-1-yl)-1-(2,2,2- |
| | trifluoroethyl)-1H-indol-5-yl]methyl}amino)piperidin-1-yl]-N-(2-methoxyethyl)-N- |
| | methylacetamide |
| 222. | 6-methanesulfonyl-N-[3-(5-{[(oxan-4-yl)amino]methyl}-1-(2,2,2-trifluoroethyl)-1H- |
| | indol-2-yl)prop-2-yn-1-yl]pyridin-3-amine |
| 223. | 2-[4-({[2-(3-{[6-(1-cyano-1-methylethyl)pyridin-3-yl]amino}prop-1-yn-1-yl)-1-(2,2,2- |
| | trifluoroethyl)-1H-indol-5-yl]methyl}amino)piperidin-1-yl]-N,N-dimethylacetamide |
| 224. | 2-methyl-2-{5-[(3-{5-[({1-[2-oxo-2-(pyrrolidin-1-yl)ethyl]piperidin-4-yl}amino)methyl]- |
| | 1-(2,2,2-trifluoroethyl)-1H-indol-2-yl}prop-2-yn-1-yl)amino]pyridin-2-yl}propanenitrile |
| 225. | 4-({[2-(3-{[6-(1-cyano-1-methylethyl)pyridin-3-yl]amino}prop-1-yn-1-yl)-1-(2,2,2- |
| | trifluoroethyl)-1H-indol-5-yl]methyl}amino)-N,N-dimethylpiperidine-1-carboxamide |
| 226. | 2-{5-[(3-{5-[({1-[2-(azetidin-1-yl)-2-oxoethyl]piperidin-4-yl}amino)-methyl]-1-(2,2,2- |
| | trifluoroethyl)-1H-indol-2-yl}prop-2-yn-1-yl)amino]-pyridin-2-yl}-2- |
| | methylpropanenitrile |
| 227. | 2-methyl-2-{5-[(3-{5-[({1-[2-(pyrrolidin-1-yl)acetyl]piperidin-4-yl}amino)methyl]-1- |
| | (2,2,2-trifluoroethyl)-1H-indol-2-yl}prop-2-yn-1-yl)amino]pyridin-2-yl}-propanenitrile |
| 228. | 2-(5-{[3-(5-{[(1-{2-[4-(dimethylamino)piperidin-1-yl]acetyl}piperidin-4-yl)amino]- |
| | methyl}-1-(2,2,2-trifluoroethyl)-1H-indol-2-yl)prop-2-yn-1-yl]amino}pyridin-2-yl)-2- |
| | methylpropanenitrile |
| 229. | 2-{5-[(3-{5-[({1-[2-(diethylamino)acetyl]piperidin-4-yl}amino)methyl]-1-(2,2,2- |
| | trifluoroethyl)-1H-indol-2-yl}prop-2-yn-1-yl)amino]pyridin-2-yl}-2-methylpropanenitrile |
| 230. | 2-methyl-2-(5-{[3-(5-{[(1-{2-[methyl(propan-2-yl)amino]acetyl}piperidin-4- |
| | yl)amino]methyl}-1-(2,2,2-trifluoroethyl)-1H-indol-2-yl)prop-2-yn-1-yl]amino}pyridin- |
| | 2-yl)propanenitrile |
| 231. | 2-methyl-2-{5-[(3-{5-[({1-[2-(pyridin-4-yl)acetyl]piperidin-4-yl}amino)methyl]-1-(2,2,2- |
| | trifluoroethyl)-1H-indol-2-yl}prop-2-yn-1-yl)amino]pyridin-2-yl}propanenitrile |
| 232. | 2-[4-({[2-(3-{[6-(1-cyano-1-methylethyl)pyridin-3-yl]amino}prop-1-yn-1-yl)-1-(2,2,2- |
| | trifluoroethyl)-1H-indol-5-yl]methyl}amino)piperidin-1-yl]-N-(pyridin-4-yl)acetamide |
| 233. | 2-methyl-2-{5-[(3-{5-[({1-[2-(morpholin-4-yl)-2-oxoethyl]piperidin-4-yl}amino)methyl]- |
| | 1-(2,2,2-trifluoroethyl)-1H-indol-2-yl}prop-2-yn-1-yl)amino]pyridin-2-yl}propanenitrile |
| 234. | 2-methyl-2-{5-[(3-{5-[({1-[2-(4-methylpiperazin-1-yl)-2-oxoethyl]piperidin-4- |
| | yl}amino)methyl]-1-(2,2,2-trifluoroethyl)-1H-indol-2-yl}prop-2-yn-1-yl)amino]pyridin- |
| | 2-yl}propanenitrile |
| 235. | 2-[4-({[2-(3-{[6-(1-cyano-1-methylethyl)pyridin-3-yl]amino}prop-1-yn-1-yl)-1-(2,2,2- |
| | trifluoroethyl)-1H-indol-5-yl]methyl}amino)piperidin-1-yl]-N-(pyridin-3-yl)acetamide |
| 236. | 2-[4-({[2-(3-{[6-(1-cyano-1-methylethyl)pyridin-3-yl]amino}prop-1-yn-1-yl)-1-(2,2,2- |
| | trifluoroethyl)-1H-indol-5-yl]methyl}amino)piperidin-1-yl]-N-(1-methylpiperidin-4- |
| | yl)acetamide |
| 237. | 2-methyl-2-[5-({3-[5-({[4-(morpholin-4-yl)cyclohexyl]amino}methyl)-1-(2,2,2- |
| | trifluoroethyl)-1H-indol-2-yl]prop-2-yn-1-yl}arnino)pyridin-2-yl]propanenitrile |
| 238. | 2-{5-[(3-{5-[({1-[2-(4-hydroxypiperidin-1-yl)acetyl]piperidin-4-yl}amino)methyl]-1- |
| | (2,2,2-trifluoroethyl)-1H-indol-2-yl}prop-2-yn-1-yl)amino]pyridin-2-yl}-2- |
| | methylpropanenitrile |
| 239. | 2-{5-[(3-{5-[({1-[2-(4-acetylpiperazin-1-yl)acetyl]piperidin-4-yl}amino)methyl]-1-(2,2,2- |
| | trifluoroethyl)-1H-indol-2-yl}prop-2-yn-1-yl)amino]pyridin-2-yl}-2-methylpropanenitrile |
| 240. | 2-(5-{[3-(5-{[(1,1-dioxo-1λ⁶-thian-4-yl)amino]methyl}-1-(2,2,2-trifluoroethyl)-1H-indol- |
| | 2-yl)prop-2-yn-1-yl]amino}pyridin-2-yl)-2-methylpropanenitrile |
| 241. | 2-{5-[(3-{5-[({1-[2-(1,1-dioxo-1λ⁶,4-thiomorpholin-4-yl)acetyl]piperidin-4- |
| | yl}amino)methyl]-1-(2,2,2-trifluoroethyl)-1H-indol-2-yl}prop-2-yn-1-yl)amino]pyridin- |
| | 2-yl}-2-methylpropanenitrile |
| 242. | 2-[5-({3-[5-({[1-(4-acetylpiperazine-1-carbonyl)piperidin-4-yl]amino}methyl)-1-(2,2,2- |
| | trifluoroethyl)-1H-indol-2-yl]prop-2-yn-1-yl}amino)pyridin-2-yl]-2-methylpropanenitrile |
| 243. | 2-(5-{[3-(5-{[(1-{2-[bis(2-hydroxyethyl)amino]acetyl}piperidin-4-yl)amino]methyl}-1- |
| | (2,2,2-trifluoroethyl)-1H-indol-2-yl)prop-2-yn-1-yl]amino}pyridin-2-yl)-2- |
| | methylpropanenitrile |
| 244. | 2-methyl-2-{5-[(3-{5-[({1-[2-(3-oxopiperazin-1-yl)acetyl]piperidin-4-yl}amino)methyl]- |
| | 1-(2,2,2-trifluoroethyl)-1H-indol-2-yl}prop-2-yn-1-yl)amino]pyridin-2-yl}propanenitrile |
| 245. | 2-methyl-2-[5-({3-[5-({[1-(morpholine-4-carbonyl)piperidin-4-yl]amino}-methyl)-1- |
| | (2,2,2-trifluoroethyl)-1H-indol-2-yl]prop-2-yn-1-yl}amino)pyridin-2-yl]propanenitrile |
| 246. | 2-methyl-2-(5-{[3-(5-{[(1-methylpiperidin-3-yl)amino]methyl}-1-(2,2,2-trifluoroethyl)- |
| | 1H-indol-2-yl)prop-2-yn-1-yl]amino}pyridin-2-yl)propanenitrile |
| 247. | N-[4-({[2-(3-{[6-(1-cyano-1-methylethyl)pyridin-3-yl]amino}prop-1-yn-1-yl)-1-(2,2,2- |
| | trifluoroethyl)-1H-indol-5-yl]methyl}amino)-cyclohexyl]acetamide |
| 248. | 2-{5-[(3-{5-[({1-[2-(1H-imidazol-1-yl)acetyl]piperidin-4-yl}amino)-methyl]-1-(2,2,2- |
| | trifluoroethyl)-1H-indol-2-yl}prop-2-yn-1-yl)amino]pyridin-2-yl}-2-methylpropanenitrile |
| 249. | 2-(5-{[3-(5-{[(1-{2-[(2-methoxyethyl)(methyl)amino]acetyl}piperidin-4- |
| | yl)amino]methyl}-1-(2,2,2-trifluoroethyl)-1H-indol-2-yl)prop-2-yn-1-yl]amino}pyridin- |
| | 2-yl)-2-methylpropanenitrile |
| 250. | N-[4-({[2-(3-{[6-(1-cyano-1-methylethyl)pyridin-3-yl]amino}prop-1-yn-1-yl)-1-(2,2,2- |
| | trifluoroethyl)-1H-indol-5-yl]methyl}amino)-cyclohexyl]methanesulfonamide |
| 251. | 2-methyl-2-(5-{[3-(5-{[(1-methyl-6-oxopiperidin-3-yl)amino]methyl}-1-(2,2,2- |
| | trifluoroethyl)-1H-indol-2-yl)prop-2-yn-1-yl]amino}pyridin-2-yl)propanenitrile |
| 252. | 2-[5-({3-[5-({[3-(dimethylamino)cyclohexyl]amino}methyl)-1-(2,2,2-trifluoroethyl)-1H- |
| | indol-2-yl]prop-2-yn-1-yl}amino)pyridin-2-yl]-2-methylpropanenitrile |
| 253. | 2-methyl-2-[5-({3-[5-({[1-(4-methylpiperazine-1-carbonyl)piperidin-4-yl]amino}methyl)- |
| | 1-(2,2,2-trifluoroethyl)-1H-indol-2-yl]prop-2-yn-1-yl}amino)pyridin-2-yl]propanenitrile |
| 254. | 2-{5-[(3-{5-[({1-[4-(dimethylamino)piperidine-1-carbonyl]piperidin-4-yl}amino)- |
| | methyl]-1-(2,2,2-trifluoroethyl)-1H-indol-2-yl}prop-2-yn-1-yl)amino]pyridin-2-yl}-2- |
| | methylpropanenitrile |
| 255. | 2-{5-[(3-{5-[({1-[2-(3-hydroxypyrrolidin-1-yl)acetyl]-piperidin-4-yl}amino)methyl]-1- |
| | (2,2,2-trifluoroethyl)-1H-indol-2-yl}prop-2-yn-1-yl)amino]pyridin-2-yl}-2- |
| | methylpropanenitrile |
| 256. | 2-{5-[(3-{5-[({1-[2-(3-methoxypyrrolidin-1-yl)acetyl]-piperidin-4-yl}amino)methyl]-1- |
| | (2,2,2-trifluoroethyl)-1H-indol-2-yl}prop-2-yn-1-yl)amino]pyridin-2-yl}-2- |
| | methylpropanenitrile |
| 257. | 2-methyl-2-[5-({3-[5-({[1-(2-{2-oxa-8-azaspiro[4.5]decan-8-yl}acetyl)-piperidin-4- |
| | yl]amino}methyl)-1-(2,2,2-trifluoroethyl)-1H-indol-2-yl]prop-2-yn-1-yl}amino)pyridin- |
| | 2-yl]propanenitrile |
| 258. | 2-{5-[(3-{5-[({1-[2-(4-hydroxy-4-methylpiperidin-1-yl)acetyl]piperidin-4- |
| | yl}amino)methyl]-1-(2,2,2-trifluoroethyl)-1H-indol-2-yl}prop-2-yn-1-yl)amino]pyridin- |
| | 2-yl}-2-methylpropanenitrile |
| 259. | 2-(5-{[3-(5-{[(1-{2-[bis(2-methoxyethyl)amino]acetyl}piperidin-4-yl)amino]methyl}-1- |
| | (2,2,2-trifluoroethyl)-1H-indol-2-yl)prop-2-yn-1-yl]amino}pyridin-2-yl)-2- |
| | methylpropanenitrile |
| 260. | 2-(5-{[3-(5-{[(1-{2-[methoxy(methyl)amino]acetyl}piperidin-4-yl)amino]methyl}-1- |
| | (2,2,2-trifluoroethyl)-1H-indol-2-yl)prop-2-yn-1-yl]amino}pyridin-2-yl)-2- |
| | methylpropanenitrile |
| 261. | 2-(5-{[3-(5-{[(1-{2-[(2,3-dihydroxypropyl)(methyl)amino]acetyl}piperidin-4- |
| | yl)amino]methyl}-1-(2,2,2-trifluoroethyl)-1H-indol-2-yl)prop-2-yn-1-yl]amino}pyridin- |
| | 2-yl)-2-methylpropanenitrile |
| 262. | 2-methyl-2-(5-{[3-(5-{[(1-methyl-2-oxopiperidin-4-yl)amino]methyl}-1-(2,2,2- |
| | trifluoroethyl)-1H-indol-2-yl)prop-2-yn-1-yl]amino}pyridin-2-yl)propanenitrile |
| 263. | 2-methyl-2-(5-{[3-(5-{[(1-{2-[methyl(1-methylpiperidin-4-yl)amino]acetyl}piperidin-4- |
| | yl)amino]methyl}-1-(2,2,2-trifluoroethyl)-1H-indol-2-yl)prop-2-yn-1-yl]amino}pyridin- |
| | 2-yl)propanenitrile |
| 264. | 2-methyl-2-[5-({3-[5-({[1-(2-{9-methyl-3,9-diazaspiro[5.5]undecan-3- |
| | yl}acetyl)piperidin-4-yl]amino}methyl)-1-(2,2,2-trifluoroethyl)-1H-indol-2-yl]prop-2-yn- |
| | 1-yl}amino)pyridin-2-yl]propanenitrile |
| 265. | 2-(5-{[3-(5-{[(1-{2-[3-(dimethyl-amino)pyrrolidin-1-yl]acetyl}piperidin-4- |
| | yl)amino]methyl}-1-(2,2,2-trifluoroethyl)-1H-indol-2-yl)prop-2-yn-1-yl]amino}pyridin- |
| | 2-yl)-2-methylpropanenitrile |
| 266. | N-[3-(5-{[(oxan-4-yl)amino]methyl}-1-(2,2,2-trifluoroethyl)-1H-indol-2-yl)prop-2-yn-1- |
| | yl]-6-(pyrrolidine-1-carbonyl)pyridin-3-amine |
| 267. | 6-(morpholine-4-carbonyl)-N-[3-(5-{[(oxan-4-yl)amino]methyl}-1-(2,2,2-trifluoroethyl)- |
| | 1H-indol-2-yl)prop-2-yn-1-yl]pyridin-3-amine |
| 268. | 2-chloro-N-[3-(5-{[(oxan-4-yl)amino]-methyl}-1-(2,2,2-trifluoroethyl)-1H-indol-2- |
| | yl)prop-2-yn-1-yl]pyrimidin-5-amine |
| 269. | 5-{[3-(5-{[(oxan-4-yl)amino]methyl}-1-(2,2,2-trifluoroethyl)-1H-indol-2-yl)prop-2-yn-1- |
| | yl]amino}-N-phenylpyridine-2-carboxamide |
| 270. | N-methyl-5-{[3-(5-([(oxan-4-yl)amino]methyl}-1-(2,2,2-trifluoroethyl)-1H-indol-2- |
| | yl)prop-2-yn-1-yl]amino]-N-(propan-2-yl)pyridine-2-carboxamide |
| 271. | 5-{[3-(5-{[(oxan-4-yl)amino]methyl}-1-(2,2,2-trifluoroethyl)-1H-indol-2-yl)prop-2-yn-1- |
| | yl]amino}-N-(pyridin-4-yl)pyridine-2-carboxamide |
| 272. | 5-{[3-(5-{[(oxan-4-yl)amino]methyl}-1-(2,2,2-trifluoroethyl)-1H-indol-2-yl)prop-2-yn-1- |
| | yl]amino}-N-(pyridin-3-yl)pyridine-2-carboxamide |
| 273. | N-(1-methylazetidin-3-yl)-5-{[3-(5-{[(oxan-4-yl)amino]methyl}-1-(2,2,2-trifluoroethyl)- |
| | 1H-indol-2-yl)prop-2-yn-1-yl]amino}pyridine-2-carboxamide |
| 274. | N,N-diethyl-5-{[3-(5-{[(oxan-4-yl)amino]methyl}-1-(2,2,2-trifluoroethyl)-1H-indol-2- |
| | yl)prop-2-yn-1-yl]amino}pyridine-2-carboxamide |
| 275. | 5-{[3-(5-{[(oxan-4-yl)amino]methyl}-1-(2,2,2-trifluoroethyl)-1H-indol-2-yl)prop-2-yn-1- |
| | yl]amino}-N-(oxetan-3-yl)pyridine-2-carboxamide |
| 276. | 1-(4-{[(2-{3-[(2-tert-butylpyrimidin-5-yl)amino]prop-1-yn-1-yl}-1-(2,2,2-trifluoroethyl)- |
| | 1H-indol-5-yl)methyl]amino}piperidin-1-yl)-2-(dimethylamino)ethan-1-one |
| 277. | 1-(4-{[(2-{3-[(6-chloropyridin-3-yl)amino]prop-1-yn-1-yl}-1-(2,2,2-trifluoroethyl)-1H- |
| | indol-5-yl)methyl]amino}piperidin-1-yl)-2-(dimethylamino)ethan-1-one |
| 278. | 5-[(3-{5-[({1-[2-(dimethylamino)-acetyl]piperidin-4-yl}amino)methyl]-1-(2,2,2- |
| | trifluoroethyl)-1H-indol-2-yl}prop-2-yn-1-yl)amino]-N-(1-methylpiperidin-4-yl)pyridine- |
| | 2-carboxamide |
| 279. | 1-(4-{[(2-{3-[(4-chloro-3-fluorophenyl)amino]prop-1-yn-1-yl}-1-(2,2,2-trifluoroethyl)- |
| | 1H-indol-5-yl)methyl]amino}piperidin-1-yl)-2-(dimethylamino)ethan-1-one |
| 280. | 2-(dimethylamino)-1-(4-{[(2-{3-[(6-methylpyridin-3-yl)amino]prop-1-yn-1-yl}-1-(2,2,2- |
| | trifluoroethyl)-1H-indol-5-yl)methyl]amino}piperidin-1-yl)ethan-1-one |
| 281. | 1-(4-{[(2-{3-[(6-tert-butylpyridin-3-yl)amino]prop-1-yn-1-yl}-1-(2,2,2-trifluoroethyl)- |
| | 1H-indol-5-yl)methyl]amino}piperidin-1-yl)-2-(dimethylamino)ethan-1-one |
| 282. | N-[6-(1-cyano-1-methylethyl)pyridin-3-yl]-3-[1-(2-fluoroethyl)-5-{[(1-methylpiperidin-4- |
| | yl)amino]methyl}-1H-indol-2-yl]prop-2-ynamide |
| 283. | 2-{5-[(3-{5-[({1-[2-(dimethylamino)acetyl]piperidin-4-yl}amino)methyl]-1-ethyl-1H- |
| | indol-2-yl}prop-2-yn-1-yl)amino]pyridin-2-yl}-2-methylpropanenitrile |
| 284. | 2-[5-({3-[1-(2,2-difluoroethyl)-5-[({1-[2-(dimethylamino)acetyl]piperidin-4- |
| | yl}amino)methyl]-1H-indol-2-yl]prop-2-yn-1-yl}amino)pyridin-2-yl]-2- |
| | methylpropanenitrile |
| 285. | 2-{5-[(3-{5-[({1-[2-(dimethylamino)acetyl]piperidin-4-yl}amino)methyl]-1-(2- |
| | fluoroethyl)-1H-indol-2-yl}prop-2-yn-1-yl)amino]pyridin-2-yl}-2-methylpropanenitrile |
| 286. | N-(6-chloropyridin-3-yl)-3-(5-{[(1-methylpiperidin-4-yl)amino]methyl}-1-(2,2,2- |
| | trifluoroethyl)-1H-indol-2-yl)prop-2-ynamide |
| 287. | 2-(5-{[3-(5-{[(1-acetylpiperidin-4-yl)amino]methyl}-1-(oxiran-2-ylmethyl)-1H-indol-2- |
| | yl)prop-2-yn-1-yl]amino}pyridin-2-yl)-2-methylpropanenitrile |
| 288. | 2-methyl-2-{5-[(3-{5-[(oxan-4-yl)amino]-1-(2,2,2-trifluoroethyl)-1H-indol-2-yl}prop-2- |
| | yn-1-yl)amino]pyridin-2-yl}propanenitrile |
| 289. | 2-{5-[(3-{4-[(1-acetylpiperidin-4-yl)amino]-1-(2,2,2-trifluoroethyl)-1H-indol-2-yl}prop- |
| | 2-yn-1-yl)amino]pyridin-2-yl}-2-methylpropanenitrile |
| 290. | 2-methyl-2-{5-[(3-{4-[(propan-2-yl)amino]-1-(2,2,2-trifluoroethyl)-1H-indol-2-yl}prop- |
| | 2-yn-1-yl)amino]pyridin-2-yl}propanenitrile |
| 291. | 2-methyl-2-{5-[(3-{4-[(piperidin-4-yl)amino]-1-(2,2,2-trifluoroethyl)-1H-indol-2- |
| | yl}prop-2-yn-1-yl)amino]pyridin-2-yl}propanenitrile |
| 292. | 2-(5-{[3-(4-{[1-(2-methoxyethyl)-piperidin-4-yl]amino}-1-(2,2,2-trifluoroethyl)-1H- |
| | indol-2-yl)prop-2-yn-1-yl]amino}pyridin-2-yl)-2-methylpropanenitrile |
| 293. | 2-{5-[(3-{4-[(1,1-dioxo-1λ⁶-thian-4-yl)amino]-1-(2,2,2-trifluoroethyl)-1H-indol-2- |
| | yl}prop-2-yn-1-yl)amino]pyridin-2-yl}-2-methylpropanenitrile |
| 294. | 3-[2-(3-{[6-(1-cyano-1-methylethyl)-pyridin-3-yl]amino}prop-1-yn-1-yl)-1-(2,2,2- |
| | trifluoroethyl)-1H-indol-4-yl]-1-(oxan-4-yl)urea |
| 295. | 3-[2-(3-{[6-(1-cyano-1-methylethyl)-pyridin-3-yl]amino}prop-1-yn-1-yl)-1-(2,2,2- |
| | trifluoroethyl)-1H-indol-4-yl]-1-(1-methylpiperidin-4-yl)urea |
| 296. | 2-(4-{[2-(3-{[6-(1-cyano-1-methylethyl)-pyridin-3-yl]amino}prop-1-yn-1-yl)-1-(2,2,2- |
| | trifluoroethyl)-1H-indol-4-yl]amino}piperidin-1-yl)-N,N-dimethylacetamide |
| 297. | 2-methyl-2-(5-{[3-(4-{[1-(propan-2-yl)piperidin-4-yl]amino}-1-(2,2,2-trifluoroethyl)-1H- |
| | indol-2-yl)prop-2-yn-1-yl]amino}pyridin-2-yl)propanenitrile |
| 298. | 2-methyl-2-(5-{[3-(4-{[1-(1-methylpiperidin-4-yl)piperidin-4-yl]amino}-1-(2,2,2- |
| | trifluoroethyl)-1H-indol-2-yl)prop-2-yn-1-yl]amino}pyridin-2-yl)propanenitrile |
| 299. | 4-{[2-(3-{[6-(1-cyano-1-methylethyl)-pyridin-3-yl]amino}prop-1-yn-1-yl)-1-(2,2,2- |
| | trifluoroethyl)-1H-indol-4-yl]amino}-N,N-dimethylpiperidine-1-carboxamide |
| 300. | N-[2-(3-{[6-(1-cyano-1-methylethyl)-pyridin-3-yl]amino}prop-1-yn-1-yl)-1-(2,2,2- |
| | trifluoroethyl)-1H-indol-4-yl]-4-methylpiperazine-1-carboxamide |
| 301. | 1-[2-(3-{[6-(1-cyano-1-methylethyl)pyridin-3-yl]amino}prop-1-yn-1-yl)-1-(2,2,2- |
| | trifluoroethyl)-1H-indol-4-yl]-3,3-dimethylurea |
| 302. | N-[2-(3-{[6-(1-cyano-1-methylethyl)-pyridin-3-yl]amino}prop-1-yn-1-yl)-1-(2,2,2- |
| | trifluoroethyl)-1H-indol-4-yl]morpholine-4-carboxamide |
| 303. | 2-{5-[(3-{4-[(4-hydroxycyclohexyl)-amino]-1-(2,2,2-trifluoroethyl)-1H-indol-2-yl}prop- |
| | 2-yn-1-yl)amino]pyridin-2-yl}-2-methylpropanenitrile |
| 304. | 2-methyl-2-[5-({3-[4-({1-[2-(4-methylpiperazin-1-yl)-2-oxoethyl]-piperidin-4-yl}amino)- |
| | 1-(2,2,2-trifluoroethyl)-1H-indol-2-yl]prop-2-yn-1-yl}amino)pyridin-2-yl]propanenitrile |
| 305. | 2-methyl-2-{5-[(3-{4-[(oxan-4-ylmethyl)amino]-1-(2,2,2-trifluoroethyl)-1H-indol-2- |
| | yl}prop-2-yn-1-yl)amino]pyridin-2-yl}propanenitrile |
| 306. | 2-{5-[(3-{4-[(1-ethylpiperidin-4-yl)amino]-1-(2,2,2-trifluoroethyl)-1H-indol-2-yl}prop-2- |
| | yn-1-yl)amino]pyridin-2-yl}-2-methylpropanenitrile |
| 307. | 2-(5-{[3-(4-{[1-(2-hydroxyethyl)piperidin-4-yl]amino}-1-(2,2,2-trifluoroethyl)-1H-indol- |
| | 2-yl)prop-2-yn-1-yl]amino}pyridin-2-yl)-2-methylpropanenitrile |
| 308. | 2-{5-[(3-{4-[(1-methanesulfonylpiperidin-4-yl)amino]-1-(2,2,2-trifluoroethyl)-1H-indol- |
| | 2-yl}prop-2-yn-1-yl)amino]pyridin-2-yl}-2-methylpropanenitrile |
| 309. | 2-(5-{[3-(4-{[1-(2-methanesulfonyl-ethyl)piperidin-4-yl]amino}-1-(2,2,2-trifluoroethyl)- |
| | 1H-indol-2-yl)prop-2-yn-1-yl]amino}pyridin-2-yl)-2-methylpropanenitrile |
| 310. | 2-methyl-2-(5-{[3-(4-{[(1R,4R)-4-hydroxycyclohexyl]amino]-1-(2,2,2-trifluoroethyl)- |
| | 1H-indol-2-yl)prop-2-yn-1-yl]amino}pyridin-2-yl)propanenitrile |
| 311. | 2-methyl-2-(5-{[3-(4-{[(1S,4S)-4-hydroxycyclohexyl]amino]-1-(2,2,2-trifluoroethyl)-1H- |
| | indol-2-yl)prop-2-yn-1-yl]amino}pyridin-2-yl)propanenitrile |
| 312. | 2-methyl-2-[5-({3-[4-({1-[2-(morpholin-4-yl)-2-oxoethyl]piperidin-4-yl}amino)-1-(2,2,2- |
| | trifluoroethyl)-1H-indol-2-yl]prop-2-yn-1-yl}amino)pyridin-2-yl]propanenitrile |
| 313. | 2-(4-{[2-(3-{[6-(1-cyano-1-methylethyl)pyridin-3-yl]amino}prop-1-yn-1-yl)-1-(2,2,2- |
| | trifluoroethyl)-1H-indol-4-yl]amino}piperidin-1-yl)-N,N-bis(2-methoxyethyl)acetamide |
| 314. | 2-methyl-2-{5-[(3-{4-[(pyrrolidin-3-yl)amino]-1-(2,2,2-trifluoroethyl)-1H-indol-2- |
| | yl}prop-2-yn-1-yl)amino]pyridin-2-yl}propanenitrile |
| 315. | 2-methyl-2-{5-[(3-{4-[(1-methylpyrrolidin-3-yl)amino]-1-(2,2,2-trifluoroethyl)-1H-indol- |
| | 2-yl}prop-2-yn-1-yl)amino]pyridin-2-yl}propanenitrile |
| 316. | 2-(4-{[2-(3-{[6-(1-cyano-1-methylethyl)pyridin-3-yl]amino}prop-1-yn-1-yl)-1-(2,2,2- |
| | trifluoroethyl)-1H-indol-4-yl]amino}piperidin-1-yl)acetamide |
| 317. | methyl 2-(4-{[2-(3-{[6-(1-cyano-1-methylethyl)pyridin-3-yl]amino}prop-1-yn-1-yl)-1- |
| | (2,2,2-trifluoroethyl)-1H-indol-4-yl]amino}piperidin-1-yl)acetate |
| 318. | 2-[5-({3-[4-({1-[2-(4-hydroxypiperidin-1-yl)-2-oxoethyl]piperidin-4-yl}amino)-1-(2,2,2- |
| | trifluoroethyl)-1H-indol-2-yl]prop-2-yn-1-yl}amino)pyridin-2-yl]-2-methylpropanenitrile |
| 319. | 2-methyl-2-{5-[(3-{4-[(2-methylpiperidin-4-yl)amino]-1-(2,2,2-trifluoroethyl)-1H-indol- |
| | 2-yl}prop-2-yn-1-yl)amino]pyridin-2-yl}propanenitrile |
| 320. | 2-{5-[(3-{4-[(1,1-dioxo-1λ⁶-thiolan-3-yl)amino]-1-(2,2,2-trifluoroethyl)-1H-indol-2- |
| | yl}prop-2-yn-1-yl)amino]pyridin-2-yl}-2-methylpropanenitrile |
| 321. | 2-methyl-2-[5-({3-[4-({1-[2-oxo-2-(pyrrolidin-1-yl)ethyl]piperidin-4-yl}amino)-1-(2,2,2- |
| | trifluoroethyl)-1H-indol-2-yl]prop-2-yn-1-yl}amino)pyridin-2-yl]propanenitrile |
| 322. | 2-{5-[(3-{4-[(1-{2-[4-(dimethylamino)piperidin-1-yl]-2-oxoethyl}piperidin-4-yl)amino]- |
| | 1-(2,2,2-trifluoroethyl)-1H-indol-2-yl}prop-2-yn-1-yl)amino]pyridin-2-yl}-2- |
| | methylpropanenitrile |
| 323. | 2-[5-({3-[4-({1-[2-(dimethylamino)acetyl]piperidin-4-yl}amino)-1-(2,2,2-trifluoroethyl)- |
| | 1H-indol-2-yl]prop-2-yn-1-yl}amino)pyridin-2-yl]-2-methylpropanenitrile |
| 324. | 2-(5-{[3-(4-{[1-(1,1-dioxo-1λ⁶-thian-4-yl)piperidin-4-yl]amino}-1-(2,2,2-trifluoroethyl)- |
| | 1H-indol-2-yl)prop-2-yn-1-yl]amino}pyridin-2-yl)-2-methylpropanenitrile |
| 325. | 2-(5-{[3-(4-{[1-(cyanomethyl)piperidin-4-yl]amino}-1-(2,2,2-trifluoroethyl)-1H-indol-2- |
| | yl)prop-2-yn-1-yl]amino}pyridin-2-yl)-2-methylpropanenitrile |
| 326. | 2-methyl-2-[5-({3-[1-(2,2,2-trifluoroethyl)-4-{[1-(2,2,2-trifluoroethyl)piperidin-4- |
| | yl]amino}-1H-indol-2-yl]prop-2-yn-1-yl}amino)pyridin-2-yl]propanenitrile |
| 327. | 2-{5-[(3-{4-[(1-{2-[4-(2-methanesulfonylethyl)piperazin-1-yl]-2-oxoethyl}piperidin-4- |
| | yl)amino]-1-(2,2,2-trifluoroethyl)-1H-indol-2-yl}prop-2-yn-1-yl)amino]pyridin-2-yl}-2- |
| | methylpropanenitrile |
| 328. | 2-[5-({3-[4-({1-[2-(1,1-dioxo-1λ⁶,4-thiomorpholin-4-yl)-2-oxoethyl]piperidin-4- |
| | yl}amino)-1-(2,2,2-trifluoroethyl)-1H-indol-2-yl]prop-2-yn-1-yl}amino)pyridin-2-yl]-2- |
| | methylpropanenitrile |
| 329. | 2-(5-{[3-(4-{[1-(1-methanesulfonylpiperidin-4-yl)piperidin-4-yl]amino}-1-(2,2,2- |
| | trifluoroethyl)-1H-indol-2-yl)prop-2-yn-1-yl]amino}pyridin-2-yl)-2-methylpropanenitrile |
| 330. | 2-(4-{[2-(3-{[6-(1-cyano-1-methylethyl)pyridin-3-yl]amino}prop-1-yn-1-yl)-1-(2,2,2- |
| | trifluoroethyl)-1H-indol-4-yl]amino}piperidin-1-yl)-N-(2,3-dihydroxypropyl)-N- |
| | methylacetamide |
| 331. | 2-(4-{[2-(3-{[6-(1-cyano-1-methylethyl)pyridin-3-yl]amino}prop-1-yn-1-yl)-1-(2,2,2- |
| | trifluoroethyl)-1H-indol-4-yl]amino}piperidin-1-yl)-N-(2,3-dihydroxypropyl)acetamide |
| 332. | 2-[5-({3-[4-({1-[2-(4-methanesulfonylpiperazin-1-yl)-2-oxoethyl]piperidin-4-yl}amino)- |
| | 1-(2,2,2-trifluoroethyl)-1H-indol-2-yl]prop-2-yn-1-yl}amino)pyridin-2-yl]-2- |
| | methylpropanenitrile |
| 333. | 2-{5-[(3-{4-[(1-{2-[4-(2-hydroxyethyl)piperazin-1-yl]-2-oxoethyl}piperidin-4-yl)amino]- |
| | 1-(2,2,2-trifluoroethyl)-1H-indol-2-yl}prop-2-yn-1-yl)amino]pyridin-2-yl}-2- |
| | methylpropanenitrile |
| 334. | 2-methyl-2-(5-{[3-(4-{[1-(oxan-4-yl)piperidin-4-yl]amino}-1-(2,2,2-trifluoroethyl)-1H- |
| | indol-2-yl)prop-2-yn-1-yl]amino}pyridin-2-yl)propanenitrile |
| 335. | 2-[5-({3-[4-({1-[1-(2-methanesulfonylethyl)piperidin-4-yl]piperidin-4-yl}amino)-1- |
| | (2,2,2-trifluoroethyl)-1H-indol-2-yl]prop-2-yn-1-yl}amino)pyridin-2-yl]-2- |
| | methylpropanenitrile |
| 336. | 2-[5-({3-[4-({1-[1-(2-methoxyethyl)piperidin-4-yl]piperidin-4-yl}amino)-1-(2,2,2- |
| | trifluoroethyl)-1H-indol-2-yl]prop-2-yn-1-yl}amino)pyridin-2-yl]-2-methylpropanenitrile |
| 337. | 2-[5-({3-[4-({1-[1-(2-hydroxyethyl)piperidin-4-yl]piperidin-4-yl}amino)-1-(2,2,2- |
| | trifluoroethyl)-1H-indol-2-yl]prop-2-yn-1-yl}amino)pyridin-2-yl]-2-methylpropanenitrile |
| 338. | 2-[5-({3-[4-({1-[2-(dimethylamino)ethyl]piperidin-4-yl}amino)-1-(2,2,2-trifluoroethyl)- |
| | 1H-indol-2-yl]prop-2-yn-1-yl}amino)pyridin-2-yl]-2-methylpropanenitrile |
| 339. | 2-(5-{[3-(4-{[1-(1-acetylpiperidin-4-yl)piperidin-4-yl]amino}-1-(2,2,2-trifluoroethyl)-1H- |
| | indol-2-yl)prop-2-yn-1-yl]amino}pyridin-2-yl)-2-methylpropanenitrile |
| 340. | 2-methyl-2-[5-({3-[4-({1-[(1R,4R)-4-hydroxycyclohexyl]piperidin-4-yl}amino)-1-(2,2,2- |
| | trifluoroethyl)-1H-indol-2-yl]prop-2-yn-1-yl}amino)pyridin-2-yl]propanenitrile |
| 341. | 2-methyl-2-[5-({3-[4-({1-[(1S,4S)-4-hydroxycyclohexyl]piperidin-4-yl}amino)-1-(2,2,2- |
| | trifluoroethyl)-1H-indol-2-yl]prop-2-yn-1-yl}amino)pyridin-2-yl]propanenitrile |
| 342. | N-(1-methylpiperidin-4-yl)-2-{3-[(6-methylpyridin-3-yl)amino]prop-1-yn-1-yl}-1-(2,2,2- |
| | trifluoroethyl)-1H-indol-4-amine |
| 343. | 2-methyl-2-{5-[(3-{4-[(1-methylpiperidin-4-yl)amino]-1-(2,2,2-trifluoroethyl)-1H-indol- |
| | 2-yl}prop-2-yn-1-yl)amino]pyridin-2-yl}propanenitrile |
| 344. | 5-[(3-{4-[(1-methylpiperidin-4-yl)amino]-1-(2,2,2-trifluoroethyl)-1H-indol-2-yl}prop-2- |
| | yn-1-yl)amino]-N-(pyridin-3-yl)pyridine-2-carboxamide |
| 345. | 5-[(3-{4-[(1-methylpiperidin-4-yl)amino]-1-(2,2,2-trifluoroethyl)-1H-indol-2-yl}prop-2- |
| | yn-1-yl)amino]pyridine-2-carbonitrile |
| 346. | N-[6-(1-cyano-1-methylethyl)pyridin-3-yl]-3-{4-[(1-methylpiperidin-4-yl)amino]-1- |
| | (2,2,2-trifluoroethyl)-1H-indol-2-yl}prop-2-ynamide |
| 347. | 2-{3-[(2-fluorophenyl)amino]prop-1-yn-1-yl}-N-(1-methylpiperidin-4-yl)-1-(2,2,2- |
| | trifluoroethyl)-1H-indol-4-amine |
| 348. | 2-{3-[(3-fluorophenyl)amino]prop-1-yn-1-yl}-N-(1-methylpiperidin-4-yl)-1-(2,2,2- |
| | trifluoroethyl)-1H-indol-4-amine |
| 349. | 4-amino-N-(3-{4-[(1-methylpiperidin-4-yl)amino]-1-(2,2,2-trifluoroethyl)-1H-indol-2- |
| | yl}prop-2-yn-1-yl)benzene-1-sulfonamide |
| 350. | 2-{3-[(6-tert-butylpyridin-3-yl)amino]prop-1-yn-1-yl}-N-(1-methylpiperidin-4-yl)-1- |
| | (2,2,2-trifluoroethyl)-1H-indol-4-amine |
| 351. | 2-{3-[(4-fluorophenyl)amino]prop-1-yn-1-yl}-N-(1-methylpiperidin-4-yl)-1-(2,2,2- |
| | trifluoroethyl)-1H-indol-4-amine |
| 352. | N,N-dimethyl-5-[(3-{4-[(1-methylpiperidin-4-yl)amino]-1-(2,2,2-trifluoroethyl)-1H- |
| | indol-2-yl}prop-2-yn-1-yl)amino]pyridine-2-carboxamide |
| 353. | 5-[(3-{4-[(1-methylpiperidin-4-yl)amino]-1-(2,2,2-trifluoroethyl)-1H-indol-2-yl}prop-2- |
| | yn-1-yl)amino]-N-(propan-2-yl)pyridine-2-carboxamide |
| 354. | N-(pyridin-3-yl)-5-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}pyridine-2-carboxamide |
| 355. | N-(pyridin-3-yl)-5-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}pyridine-2-carboxamide |
| 356. | 2-{3-[(6-methanesulfonylpyridin-3-yl)amino]prop-1-yn-1-yl}-N-(1-methylpiperidin-4- |
| | yl)-1-(2,2,2-trifluoroethyl)-1H-indol-4-amine |
| 357. | 6-tert-butyl-N-(3-{4-[(1-methylpiperidin-4-yl)amino]-1-(2,2,2-trifluoroethyl)-1H-indol-2- |
| | yl}prop-2-yn-1-yl)pyridine-3-carboxamide |
| 358. | 2-{3-[(6-chloropyridin-3-yl)amino]prop-1-yn-1-yl}-N-(1-methylpiperidin-4-yl)-1-(2,2,2- |
| | trifluoroethyl)-1H-indol-4-amine |
| 359. | 2-{4-[(3-{4-[(1-methylpiperidin-4-yl)amino]-1-(2,2,2-trifluoroethyl)-1H-indol-2-yl}prop- |
| | 2-yn-1-yl)amino]phenyl}propan-2-ol |
| 360. | 6-methyl-N-(3-{4-[(1-methylpiperidin-4-yl)amino]-1-(2,2,2-trifluoroethyl)-1H-indol-2- |
| | yl}prop-2-yn-1-yl)pyridine-3-carboxamide |
| 361. | N-(1-methylpiperidin-4-yl)-1-(2,2,2-trifluoroethyl)-2-(3-{[6-(trifluoromethyl)pyridin-3- |
| | yl]amino}prop-1-yn-1-yl)-1H-indol-4-amine |
| 362. | 3-(3-{4-[(1-methylpiperidin-4-yl)amino]-1-(2,2,2-trifluoroethyl)-1H-indol-2-yl}prop-2- |
| | yn-1-yl)-1-phenylurea |
| 363. | 2-{3-[(4-tert-butyl-2-fluorophenyl)-amino]prop-1-yn-1-yl}-N-(1-methylpiperidin-4-yl)-1- |
| | (2,2,2-trifluoroethyl)-1H-indol-4-amine |
| 364. | 2-{3-fluoro-4-[(3-{4-[(1-methylpiperidin-4-yl)amino]-1-(2,2,2-trifluoroethyl)-1H-indol-2- |
| | yl}prop-2-yn-1-yl)amino]phenyl}-2-methylpropanenitrile |
| 365. | 4-[(3-{4-[(1-methylpiperidin-4-yl)amino]-1-(2,2,2-trifluoroethyl)-1H-indol-2-yl}prop-2- |
| | yn-1-yl)amino]benzene-1-sulfonamide |
| 366. | 2-{3-[(2,6-difluoro-4-methane-sulfonylphenyl)amino]prop-1-yn-1-yl}-N-(1- |
| | methylpiperidin-4-yl)-1-(2,2,2-trifluoroethyl)-1H-indol-4-amine |
| 367. | N-methyl-4-[(3-{4-[(1-methylpiperidin-4-yl)amino]-1-(2,2,2-trifluoroethyl)-1H-indol-2- |
| | yl}prop-2-yn-1-yl)amino]benzene-1-sulfonamide |
| 368. | 2-{3-[(4-methanesulfonyl-3-methoxyphenyl)amino]prop-1-yn-1-yl}-N-(1- |
| | methylpiperidin-4-yl)-1-(2,2,2-trifluoroethyl)-1H-indol-4-amine |
| 369. | 2-{3-[(4-methanesulfonyl-2-methoxyphenyl)amino]prop-1-yn-1-yl}-N-(1- |
| | methylpiperidin-4-yl)-1-(2,2,2-trifluoroethyl)-1H-indol-4-amine |
| 370. | 2-{3-[(5-methanesulfonylpyridin-2-yl)amino]prop-1-yn-1-yl}-N-(1-methylpiperidin-4- |
| | yl)-1-(2,2,2-trifluoroethyl)-1H-indol-4-amine |
| 371. | 2-{3-[(4-methanesulfonyl-2-methylphenyl)amino]prop-1-yn-1-yl}-N-(1-methylpiperidin- |
| | 4-yl)-1-(2,2,2-trifluoroethyl)-1H-indol-4-amine |
| 372. | methyl 3-methoxy-4-[(3-{4-[(1-methylpiperidin-4-yl)amino]-1-(2,2,2-trifluoroethyl)-1H- |
| | indol-2-yl}prop-2-yn-1-yl)amino]benzoate |
| 373. | N-{3-methoxy-4-[(3-{4-[(1-methylpiperidin-4-yl)amino]-1-(2,2,2-trifluoroethyl)-1H- |
| | indol-2-yl}prop-2-yn-1-yl)amino]phenyl}methanesulfonamide |
| 374. | 3-methoxy-4-[(3-{4-[(1-methylpiperidin-4-yl)amino]-1-(2,2,2-trifluoroethyl)-1H-indol-2- |
| | yl}prop-2-yn-1-yl)amino]benzonitrile |
| 375. | 3-methoxy-4-[(3-{4-[(1-methylpiperidin-4-yl)amino]-1-(2,2,2-trifluoroethyl)-1H-indol-2- |
| | yl}prop-2-yn-1-yl)amino]benzoic acid |
| 376. | 2-{3-[(2,4-dimethoxyphenyl)amino]prop-1-yn-1-yl}-N-(1-methylpiperidin-4-yl)-1-(2,2,2- |
| | trifluoroethyl)-1H-indol-4-amine |
| 377. | 2-{3-[(2-methoxypyridin-3-yl)amino]prop-1-yn-1-yl}-N-(1-methylpiperidin-4-yl)-1- |
| | (2,2,2-trifluoroethyl)-1H-indol-4-amine |
| 378. | 2-{3-[(5-fluoro-2-methoxyphenyl)-amino]prop-1-yn-1-yl}-N-(1-methylpiperidin-4-yl)-1- |
| | (2,2,2-trifluoroethyl)-1H-indol-4-amine |
| 379. | 2-{3-[(2-ethoxy-4-methane-sulfonylphenyl)amino]prop-1-yn-1-yl}-N-(1-methylpiperidin- |
| | 4-yl)-1-(2,2,2-trifluoroethyl)-1H-indol-4-amine |
| 380. | 2-{3-[(3-fluoro-2-methoxyphenyl)-amino]prop-1-yn-1-yl}-N-(1-methylpiperidin-4-yl)-1- |
| | (2,2,2-trifluoroethyl)-1H-indol-4-amine |
| 381. | 3-methoxy-4-[(3-{4-[(1-methylpiperidin-4-yl)amino]-1-(2,2,2-trifluoroethyl)-1H-indol-2- |
| | yl}prop-2-yn-1-yl)amino]benzene-1-sulfonamide |
| 382. | 2-{3-[(4-fluoro-2-methoxyphenyl)-amino]prop-1-yn-1-yl}-N-(1-methylpiperidin-4-yl)-1- |
| | (2,2,2-trifluoroethyl)-1H-indol-4-amine |
| 383. | 3-methoxy-4-[(3-{4-[(1-methylpiperidin-4-yl)amino]-1-(2,2,2-trifluoroethyl)-1H-indol-2- |
| | yl}prop-2-yn-1-yl)amino]-benzamide |
| 384. | 2-{3-[(2-fluoro-6-methoxyphenyl)-amino]prop-1-yn-1-yl}-N-(1-methylpiperidin-4-yl)-1- |
| | (2,2,2-trifluoroethyl)-1H-indol-4-amine |
| 385. | 2-{3-[(4-tert-butyl-2-methoxyphenyl)-amino]prop-1-yn-1-yl}-N-(1-methylpiperidin-4- |
| | yl)-1-(2,2,2-trifluoroethyl)-1H-indol-4-amine |
| 386. | 4-methoxy-3-[(3-{4-[(1-methylpiperidin-4-yl)amino]-1-(2,2,2-trifluoroethyl)-1H-indol-2- |
| | yl}prop-2-yn-1-yl)amino]-benzonitrile |
| 387. | 2-{3-[(5-tert-butyl-2-methoxyphenyl)-amino]prop-1-yn-1-yl}-N-(1-methylpiperidin-4- |
| | yl)-1-(2,2,2-trifluoroethyl)-1H-indol-4-amine |
| 388. | N-(1-methylpiperidin-4-yl)-2-[3-(phenylamino)prop-1-yn-1-yl]-1-(2,2,2-trifluoroethyl)- |
| | 1H-indol-4-amine |
| 389. | 5-methanesulfonyl-2-[(3-{4-[(1-methylpiperidin-4-yl)amino]-1-(2,2,2-trifluoroethyl)-1H- |
| | indol-2-yl}prop-2-yn-1-yl)amino]benzonitrile |
| 390. | 2-(3-{[2-(2-fluoroethoxy)-4-methanesulfonylphenyl]amino}prop-1-yn-1-yl)-N-(1- |
| | methylpiperidin-4-yl)-1-(2,2,2-trifluoroethyl)-1H-indol-4-amine |
| 391. | 2-{3-[(3-methoxypyridin-4-yl)amino]prop-1-yn-1-yl}-N-(1-methylpiperidin-4-yl)-1- |
| | (2,2,2-trifluoroethyl)-1H-indol-4-amine |
| 392. | 2-{3-[(2-chloro-4-methanesulfonyl-phenyl)amino]prop-1-yn-1-yl}-N-(1-methylpiperidin- |
| | 4-yl)-1-(2,2,2-trifluoroethyl)-1H-indol-4-amine |
| 393. | 2-{3-[(4-methoxyphenyl)amino]prop-1-yn-1-yl}-N-(1-methylpiperidin-4-yl)-1-(2,2,2- |
| | trifluoroethyl)-1H-indol-4-amine |
| 394. | 2-(3-{[4-methanesulfonyl-2-(2-methoxyethoxy)phenyl]amino}prop-1-yn-1-yl)-N-(1- |
| | methylpiperidin-4-yl)-1-(2,2,2-trifluoroethyl)-1H-indol-4-amine |
| 395. | 5-[(3-{4-[(1,1-dioxo-1λ⁶-thian-4-yl)amino]-1-(2,2,2-trifluoroethyl)-1H-indol-2-yl}prop-2- |
| | yn-1-yl)amino]pyridine-2-carbonitrile |
| 396. | 4-{[2-(3-{[6-(morpholine-4-carbonyl)pyridin-3-yl]amino}prop-1-yn-1-yl)-1-(2,2,2- |
| | trifluoroethyl)-1H-indol-4-yl]amino}-1λ⁶-thiane-1,1-dione |
| 397. | 4-{[2-(3-{[6-(4-methylpiperazine-1-carbonyl)pyridin-3-yl]amino}prop-1-yn-1-yl)-1- |
| | (2,2,2-trifluoroethyl)-1H-indol-4-yl]amino}-1λ⁶-thiane-1,1-dione |
| 398. | 4-[(2-{3-[(quinolin-3-yl)amino]prop-1-yn-1-yl}-1-(2,2,2-trifluoroethyl)-1H-indol-4- |
| | yl)amino]-1λ⁶-thiane-1,1-dione |
| 399. | 4-[(2-{3-[(quinoxalin-6-yl)amino]prop-1-yn-1-yl}-1-(2,2,2-trifluoroethyl)-1H-indol-4- |
| | yl)amino]-1λ⁶-thiane-1,1-dione |
| 400. | 4-[(2-{3-[(4-methanesulfonylphenyl)-amino]prop-1-yn-1-yl}-1-(2,2,2-trifluoroethyl)-1H- |
| | indol-4-yl)amino]-1λ⁶-thiane-1,1-dione |
| 401. | 5-[(3-{4-[(1,1-dioxo-1λ⁶-thian-4-yl)amino]-1-(2,2,2-trifluoroethyl)-1H-indol-2-yl}prop-2- |
| | yn-1-yl)amino]pyridine-2-carboxamide |
| 402. | 4-[(2-{3-[(6-methoxypyridin-3-yl)amino]prop-1-yn-1-yl}-1-(2,2,2-trifluoroethyl)-1H- |
| | indol-4-yl)amino]-1λ⁶-thiane-1,1-dione |
| 403. | 4-{[2-(3-{[6-(4-hydroxypiperidine-1-carbonyl)pyridin-3-yl]amino}prop-1-yn-1-yl)-1- |
| | (2,2,2-trifluoroethyl)-1H-indol-4-yl]amino}-1λ⁶-thiane-1,1-dione |
| 404. | 4-[(2-{3-[(1-methyl-2-oxo-1,2-dihydropyridin-4-yl)amino]prop-1-yn-1-yl}-1-(2,2,2- |
| | trifluoroethyl)-1H-indol-4-yl)amino]-1λ⁶-thiane-1,1-dione |
| 405. | 4-[(2-{3-[(2-methoxypyridin-4-yl)amino]prop-1-yn-1-yl}-1-(2,2,2-trifluoroethyl)-1H- |
| | indol-4-yl)amino]-1λ⁶-thiane-1,1-dione |
| 406. | 2-{4-[(3-{4-[(1,1-dioxo-1λ⁶-thian-4-yl)amino]-1-(2,2,2-trifluoroethyl)-1H-indol-2- |
| | yl}prop-2-yn-1-yl)amino]-3-fluorophenyl}-2-methylpropanenitrile |
| 407. | 5-[(3-{4-[(1,1-dioxo-1λ⁶-thian-4-yl)amino]-1-(2,2,2-trifluoroethyl)-1H-indol-2-yl}prop-2- |
| | yn-1-yl)amino]-N-(1-methylpiperidin-4-yl)pyridine-2-carboxamide |
| 408. | 4-[(2-{3-[(2-fluoro-4-methanesulfonyl-phenyl)amino]prop-1-yn-1-yl}-1-(2,2,2- |
| | trifluoroethyl)-1H-indol-4-yl)amino]-1λ⁶-thiane-1,1-dione |
| 409. | 4-{[2-(3-{[4-(ethanesulfonyl)-phenyl]amino}prop-1-yn-1-yl)-1-(2,2,2-trifluoroethyl)-1H- |
| | indol-4-yl]amino}-1λ⁶-thiane-1,1-dione |
| 410. | 4-[(2-{3-[(2-tert-butylpyrimidin-5-yl)amino]prop-1-yn-1-yl}-1-(2,2,2-trifluoroethyl)-1H- |
| | indol-4-yl)amino]-1λ⁶-thiane-1,1-dione |
| 411. | 3-{4-[(1,1-dioxo-1λ⁶-thian-4-yl)amino]-1-(2,2,2-trifluoroethyl)-1H-indol-2-yl}-N-(4- |
| | methanesulfonyl-phenyl)-prop-2-ynamide |
| 412. | 5-[(3-{4-[(1,1-dioxo-1λ⁶-thian-4-yl)amino]-1-(2,2,2-trifluoroethyl)-1H-indol-2-yl}prop-2- |
| | yn-1-yl)amino]-N-(oxan-4-yl)pyridine-2-carboxamide |
| 413. | 5-[(3-{4-[(1,1-dioxo-1λ⁶-thian-4-yl)amino]-1-(2,2,2-trifluoroethyl)-1H-indol-2-yl}prop-2- |
| | yn-1-yl)amino]-N-(pyridin-3-yl)pyridine-2-carboxamide |
| 414. | 5-[(3-{4-[(1,1-dioxo-1λ⁶-thian-4-yl)amino]-1-(2,2,2-trifluoroethyl)-1H-indol-2-yl}prop-2- |
| | yn-1-yl)amino]-N-methylpyridine-2-carboxamide |
| 415. | 4-[(2-{3-[(3-chloro-4-methanesulfonylphenyl)amino]prop-1-yn-1-yl}-1-(2,2,2- |
| | trifluoroethyl)-1H-indol-4-yl)amino]-1λ⁶-thiane-1,1-dione |
| 416. | N-(2,3-dihydroxypropyl)-5-[(3-{4-[(1,1-dioxo-1λ⁶-thian-4-yl)amino]-1-(2,2,2- |
| | trifluoroethyl)-1H-indol-2-yl}prop-2-yn-1-yl)amino]pyridine-2-carboxamide |
| 417. | 5-[(3-{4-[(1,1-dioxo-1λ⁶-thian-4-yl)amino]-1-(2,2,2-trifluoroethyl)-1H-indol-2-yl}prop-2- |
| | yn-1-yl)amino]-N-hydroxypyridine-2-carboxamide |
| 418. | 5-[(3-{4-[(1,1-dioxo-1λ⁶-thian-4-yl)amino]-1-(2,2,2-trifluoroethyl)-1H-indol-2-yl}prop-2- |
| | yn-1-yl)amino]-N-(2-hydroxyethyl)pyridine-2-carboxamide |
| 419. | 5-[(3-{4-[(1,1-dioxo-1λ⁶-thian-4-yl)amino]-1-(2,2,2-trifluoroethyl)-1H-indol-2-yl}prop-2- |
| | yn-1-yl)amino]-N-hydroxy-N-methylpyridine-2-carboxamide |
| 420. | 4-amino-N-(3-{4-[(1,1-dioxo-1λ⁶-thian-4-yl)amino]-1-(2,2,2-trifluoroethyl)-1H-indol-2- |
| | yl}prop-2-yn-1-yl)benzene-1-sulfonamide |
| 421. | 4-({2-[3-({pyrido[2,3-b]pyrazin-7-yl}amino)prop-1-yn-1-yl]-1-(2,2,2-trifluoroethyl)-1H- |
| | indol-4-yl}amino)-1λ⁶-thiane-1,1-dione |
| 422. | 4-[(3-{4-[(1,1-dioxo-1λ⁶-thian-4-yl)amino]-1-(2,2,2-trifluoroethyl)-1H-indol-2-yl}prop-2- |
| | yn-1-yl)amino]-benzamide |
| 423. | 4-{[2-(3-{[2-(methylsulfanyl)pyrimidin-5-yl]amino}prop-1-yn-1-yl)-1-(2,2,2- |
| | trifluoroethyl)-1H-indol-4-yl]amino}-1λ⁶-thiane-1,1-dione |
| 424. | 4-[(3-{4-[(1,1-dioxo-1λ⁶-thian-4-yl)amino]-1-(2,2,2-trifluoroethyl)-1H-indol-2-yl}prop-2- |
| | yn-1-yl)amino]benzene-1-sulfonamide |
| 425. | 4-{[2-(3-{[4-(2-methylpropane-2-sulfonyl)phenyl]amino}prop-1-yn-1-yl)-1-(2,2,2- |
| | trifluoroethyl)-1H-indol-4-yl]amino}-1λ⁶-thiane-1,1-dione |
| 426. | 4-[(3-{4-[(1,1-dioxo-1λ⁶-thian-4-yl)amino]-1-(2,2,2-trifluoroethyl)-1H-indol-2-yl}prop-2- |
| | yn-1-yl)amino]-N,N-dimethylbenzene-1-sulfonamide |
| 427. | 4-{[1-(2,2,2-trifluoroethyl)-2-[2-(trimethylsilyl)ethynyl]-1H-indol-4-yl]amino}-1λ⁶- |
| | thiane-1,1-dione |
| 428. | 4-[(2-{3-[(5-methanesulfonylpyridin-2-yl)amino]prop-1-yn-1-yl}-1-(2,2,2-trifluoroethyl)- |
| | 1H-indol-4-yl)amino]-1λ⁶-thiane-1,1-dione |
| 429. | 4-[(2-{3-[(4-methanesulfonyl-2-methoxyphenyl)amino]prop-1-yn-1-yl}-1-(2,2,2- |
| | trifluoroethyl)-1H-indol-4-yl)amino]-1λ⁶-thiane-1,1-dione |
| 430. | 4-[(3-{4-[(1,1-dioxo-1λ⁶-thian-4-yl)amino]-1-(2,2,2-trifluoroethyl)-1H-indol-2-yl}prop-2- |
| | yn-1-yl)amino]-N-methylbenzene-1-sulfonamide |
| 431. | 4-{[2-ethynyl-1-(2,2,2-trifluoroethyl)-1H-indol-4-yl]amino}-1λ⁶-thiane-1,1-dione |
| 432. | N-{4-[(3-{4-[(1,1-dioxo-1λ⁶-thian-4-yl)amino]-1-(2,2,2-trifluoroethyl)-1H-indol-2- |
| | yl}prop-2-yn-1-yl)amino]-3-methoxyphenyl}methanesulfonamide |
| 433. | 4-[(3-{4-[(1,1-dioxo-1λ⁶-thian-4-yl)amino]-1-(2,2,2-trifluoroethyl)-1H-indol-2-yl}prop-2- |
| | yn-1-yl)amino]-3-methoxybenzoic acid |
| 434. | 4-[(3-{4-[(1,1-dioxo-1λ⁶-thian-4-yl)amino]-1-(2,2,2-trifluoroethyl)-1H-indol-2-yl}prop-2- |
| | yn-1-yl)amino]-3-methoxybenzonitrile |
| 435. | 4-[(2-{3-[(5-fluoro-2-methoxyphenyl)amino]prop-1-yn-1-yl}-1-(2,2,2-trifluoroethyl)-1H- |
| | indol-4-yl)amino]-1λ⁶-thiane-1,1-dione |
| 436. | 4-[(2-{3-[(2-methoxy-6-methylpyridin-3-yl)amino]prop-1-yn-1-yl}-1-(2,2,2- |
| | trifluoroethyl)-1H-indol-4-yl)amino]-1λ⁶-thiane-1,1-dione |
| 437. | 4-[(2-{3-[(2-hydroxy-6-methylpyridin-3-yl)amino]prop-1-yn-1-yl}-1-(2,2,2- |
| | trifluoroethyl)-1H-indol-4-yl)amino]-1λ⁶-thiane-1,1-dione |
| 438. | 4-[(3-{4-[(1,1-dioxo-1λ⁶-thian-4-yl)amino]-1-(2,2,2-trifluoroethyl)-1H-indol-2-yl}prop-2- |
| | yn-1-yl)amino]-3-methoxybenzamide |
| 439. | 4-[(2-{3-[(2-methoxypyridin-3-yl)amino]prop-1-yn-1-yl}-1-(2,2,2-trifluoroethyl)-1H- |
| | indol-4-yl)amino]-1λ⁶-thiane-1,1-dione |
| 440. | 4-[(2-{3-[(4-fluoro-2-methoxyphenyl)amino]prop-1-yn-1-yl}-1-(2,2,2-trifluoroethyl)-1H- |
| | indol-4-yl)amino]-1λ⁶-thiane-1,1-dione |
| 441. | 4-[(2-{3-[(5-tert-butyl-2-methoxyphenyl)amino]prop-1-yn-1-yl}-1-(2,2,2-trifluoroethyl)- |
| | 1H-indol-4-yl)amino]-1λ⁶-thiane-1,1-dione |
| 442. | 4-[(2-{3-[(2-ethoxy-4-methanesulfonylphenyl)amino]prop-1-yn-1-yl}-1-(2,2,2- |
| | trifluoroethyl)-1H-indol-4-yl)amino]-1λ⁶-thiane-1,1-dione |
| 443. | 4-[(2-{3-[(3-fluoro-2-methoxyphenyl)-amino]prop-1-yn-1-yl}-1-(2,2,2-trifluoroethyl)- |
| | 1H-indol-4-yl)amino]-1λ⁶-thiane-1,1-dione |
| 444. | 4-({2-[3-(methylamino)prop-1-yn-1-yl]-1-(2,2,2-trifluoroethyl)-1H-indol-4-yl}amino)- |
| | 1λ⁶-thiane-1,1-dione |
| 445. | 4-[(3-{4-[(1,1-dioxo-1λ⁶-thian-4-yl)amino]-1-(2,2,2-trifluoroethyl)-1H-indol-2-yl}prop-2- |
| | yn-1-yl)amino]-3-methoxybenzene-1-sulfonamide |
| 446. | 4-[(2-{3-[(2-fluoro-6-methoxyphenyl)-amino]prop-1-yn-1-yl}-1-(2,2,2-trifluoroethyl)- |
| | 1H-indol-4-yl)amino]-1λ⁶-thiane-1,1-dione |
| 447. | 3-[(3-{4-[(1,1-dioxo-1λ⁶-thian-4-yl)amino]-1-(2,2,2-trifluoroethyl)-1H-indol-2-yl}prop-2- |
| | yn-1-yl)amino]-4-methoxybenzonitrile |
| 448. | 4-[(2-{3-[(4-tert-butyl-2-methoxy-phenyl)amino]prop-1-yn-1-yl}-1-(2,2,2-trifluoroethyl)- |
| | 1H-indol-4-yl)amino]-1λ⁶-thiane-1,1-dione |
| 449. | 4-({2-[3-(phenylamino)prop-1-yn-1-yl]-1-(2,2,2-trifluoroethyl)-1H-indol-4-yl}amino)- |
| | 1λ⁶-thiane-1,1-dione |
| 450. | 4-{[2-(3-{[2-(2-fluoroethoxy)-4-methanesulfonylphenyl]amino}prop-1-yn-1-yl)-1-(2,2,2- |
| | trifluoroethyl)-1H-indol-4-yl]amino}-1λ⁶-thiane-1,1-dione |
| 451. | 2-[(3-{4-[(1,1-dioxo-1λ⁶-thian-4-yl)amino]-1-(2,2,2-trifluoroethyl)-1H-indol-2-yl}prop-2- |
| | yn-1-yl)amino]-5-methanesulfonylbenzonitrile |
| 452. | 4-[(2-{3-[(2-chloro-4-methanesulfonyl-phenyl)amino]prop-1-yn-1-yl}-1-(2,2,2- |
| | trifluoroethyl)-1H-indol-4-yl)amino]-1λ⁶-thiane-1,1-dione |
| 453. | 4-[(2-{3-[(4-methoxyphenyl)amino]prop-1-yn-1-yl}-1-(2,2,2-trifluoroethyl)-1H-indol-4- |
| | yl)amino]-1λ⁶-thiane-1,1-dione |
| 454. | 4-{[2-(3-{[4-methanesulfonyl-2-(2-methoxyethoxy)phenyl]amino}prop-1-yn-1-yl)-1- |
| | (2,2,2-trifluoroethyl)-1H-indol-4-yl]amino}-1λ⁶-thiane-1,1-dione |
| 455. | 2-{4-[(2-{3-[(4-methanesulfonylphenyl)amino]prop-1-yn-1-yl}-1-(2,2,2-trifluoroethyl)- |
| | 1H-indol-4-yl)amino]piperidin-1-yl}-N,N-dimethylacetamide |
| 456. | 2-methyl-2-(5-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}pyridin-2-yl)propanenitrile |
| 457. | 2-(5-((3-(4-(((1S,4S)-4-(dimethylamino)-cyclohexyl)amino)-1-(2,2,2-trifluoroethyl)-1H- |
| | indol-2-yl)prop-2-yn-1-yl)amino)pyridin-2-yl)-2-methylpropanenitrile |
| 458. | N-[1-(2-methanesulfonylethyl)piperidin-4-yl]-2-{3-[(4-methanesulfonylphenyl)- |
| | amino]prop-1-yn-1-yl}-1-(2,2,2-trifluoroethyl)-1H-indol-4-amine |
| 459. | 5-({3-[4-({1-[(dimethylcarbamoyl)methyl]piperidin-4-yl}amino)-1-(2,2,2-trifluoroethyl)- |
| | 1H-indol-2-yl]prop-2-yn-1-yl}amino)pyridine-2-carboxamide |
| 460. | 5-{[3-(4-{[1-(2-methanesulfonylethyl)piperidin-4-yl]amino}-1-(2,2,2-trifluoroethyl)-1H- |
| | indol-2-yl)prop-2-yn-1-yl]amino}pyridine-2-carboxamide |
| 461. | 5-[(3-{4-[(piperidin-4-yl)amino]-1-(2,2,2-trifluoroethyl)-1H-indol-2-yl}prop-2-yn-1- |
| | yl)amino]pyridine-2-carboxamide |
| 462. | 5-{[3-(4-{[1-(carbamoylmethyl)piperidin-4-yl]amino}-1-(2,2,2-trifluoroethyl)-1H-indol- |
| | 2-yl)prop-2-yn-1-yl]amino}pyridine-2-carboxamide |
| 463. | 5-{[3-(4-{[1-(2-hydroxyethyl)piperidin-4-yl]amino}-1-(2,2,2-trifluoroethyl)-1H-indol-2- |
| | yl)prop-2-yn-1-yl]amino}pyridine-2-carboxamide |
| 464. | 5-{[3-(4-{[1-(oxan-4-yl)piperidin-4-yl]amino}-1-(2,2,2-trifluoroethyl)-1H-indol-2- |
| | yl)prop-2-yn-1-yl]amino}pyridine-2-carboxamide |
| 465. | 5-{[3-(4-{[(1R,4R)-4-(dimethylamino)-cyclohexyl]amino}-1-(2,2,2-trifluoroethyl)-1H- |
| | indol-2-yl)prop-2-yn-1-yl]amino}pyridine-2-carboxamide |
| 466. | 4-{[3-(4-{[1-(2-methanesulfonyl-ethyl)piperidin-4-yl]amino}-1-(2,2,2-trifluoroethyl)-1H- |
| | indol-2-yl)prop-2-yn-1-yl]amino}benzene-1-sulfonamide |
| 467. | 4-{[3-(4-{[1-(2-methoxyethyl)piperidin-4-yl]amino}-1-(2,2,2-trifluoroethyl)-1H-indol-2- |
| | yl)prop-2-yn-1-yl]amino}benzene-1-sulfonamide |
| 468. | 5-{[3-(4-{[(1S,4S)-4-(dimethylamino)-cyclohexyl]amino}-1-(2,2,2-trifluoroethyl)-1H- |
| | indol-2-yl)prop-2-yn-1-yl]amino}pyridine-2-carboxamide |
| 469. | N,N-dimethyl-4-{[3-(4-{[1-(oxan-4-yl)piperidin-4-yl]amino}-1-(2,2,2-trifluoroethyl)-1H- |
| | indol-2-yl)prop-2-yn-1-yl]amino}benzene-1-sulfonamide |
| 470. | 4-{[3-(4-{[1-(oxan-4-yl)piperidin-4-yl]amino}-1-(2,2,2-trifluoroethyl)-1H-indol-2- |
| | yl)prop-2-yn-1-yl]amino}benzene-1-sulfonamide |
| 471. | 2-{4-[(2-{3-[(4-sulfamoylphenyl)-amino]prop-1-yn-1-yl}-1-(2,2,2-trifluoroethyl)-1H- |
| | indol-4-yl)amino]-piperidin-1-yl}acetamide |
| 472. | 4-{[3-(4-{[1-(2-methoxyethyl)piperidin-4-yl]amino}-1-(2,2,2-trifluoroethyl)-1H-indol-2- |
| | yl)prop-2-yn-1-yl]amino}-N,N-dimethylbenzene-1-sulfonamide |
| 473. | 4-[(3-{4-[(oxan-4-yl)amino]-1-(2,2,2-trifluoroethyl)-1H-indol-2-yl}prop-2-yn-1- |
| | yl)amino]benzene-1-sulfonamide |
| 474. | 4-{[3-(4-{[1-(2-hydroxypropyl)piperidin-4-yl]amino}-1-(2,2,2-trifluoroethyl)-1H-indol-2- |
| | yl)prop-2-yn-1-yl]amino}benzene-1-sulfonamide |
| 475. | 4-{[3-(4-{[1-(2,3-dihydroxypropyl)piperidin-4-yl]amino}-1-(2,2,2-trifluoroethyl)-1H- |
| | indol-2-yl)prop-2-yn-1-yl]amino}benzene-1-sulfonamide |
| 476. | 4-({3-[4-({1-[2-(morpholin-4-yl)-2-oxoethyl]piperidin-4-yl}amino)-1-(2,2,2- |
| | trifluoroethyl)-1H-indol-2-yl]prop-2-yn-1-yl}amino)benzene-1-sulfonamide |
| 477. | methyl 2-{4-[(2-{3-[(4-sulfamoylphenyl)-amino]prop-1-yn-1-yl}-1-(2,2,2-trifluoro- |
| | ethyl)-1H-indol-4-yl)amino]piperidin-1-yl}acetate |
| 478. | 4-{[3-(4-{[1-(2-hydroxyethyl)piperidin-4-yl]amino}-1-(2,2,2-trifluoroethyl)-1H-indol-2- |
| | yl)prop-2-yn-1-yl]amino}benzene-1-sulfonamide |
| 479. | 4-({3-[4-({1-[2-(2-hydroxyethoxy)ethyl]piperidin-4-yl}amino)-1-(2,2,2-trifluoroethyl)- |
| | 1H-indol-2-yl]prop-2-yn-1-yl}amino)benzene-1-sulfonamide |
| 480. | N,N-dimethyl-2-{4-[(2-{3-[(4-sulfamoylphenyl)amino]prop-1-yn-1-yl}-1-(2,2,2- |
| | trifluoroethyl)-1H-indol-4-yl)amino]piperidin-1-yl}acetamide |
| 481. | 4-({3-[4-({1-[2-(4-hydroxypiperidin-1-yl)-2-oxoethyl]piperidin-4-yl}amino)-1-(2,2,2- |
| | trifluoroethyl)-1H-indol-2-yl]prop-2-yn-1-yl}amino)benzene-1-sulfonamide |
| 482. | 2-{4-[(2-{3-[(4-sulfamoylphenyl)amino]prop-1-yn-1-yl}-1-(2,2,2-trifluoroethyl)-1H- |
| | indol-4-yl)amino]piperidin-1-yl}acetic acid |
| 483. | 4-({3-[4-({1-[2-(4-methylpiperazin-1-yl)-2-oxoethyl]piperidin-4-yl}amino)-1-(2,2,2- |
| | trifluoroethyl)-1H-indol-2-yl]prop-2-yn-1-yl}amino)benzene-1-sulfonamide |
| 484. | N-methyl-2-{4-[(2-{3-[(4-sulfamoylphenyl)amino]prop-1-yn-1-yl}-1-(2,2,2- |
| | trifluoroethyl)-1H-indol-4-yl)amino]piperidin-1-yl}acetamide |
| 485. | N-methyl-4-[(3-{4-[(oxan-4-yl)amino]-1-(2,2,2-trifluoroethyl)-1H-indol-2-yl}prop-2-yn- |
| | 1-yl)amino]benzene-1-sulfonamide |
| 486. | 4-{[3-(4-{[1-(2-methoxyethyl)piperidin-4-yl]amino}-1-(2,2,2-trifluoroethyl)-1H-indol-2- |
| | yl)prop-2-yn-1-yl]amino}-N-methylbenzene-1-sulfonamide |
| 487. | N-methyl-4-{[3-(4-{[1-(oxan-4-yl)piperidin-4-yl]amino}-1-(2,2,2-trifluoroethyl)-1H- |
| | indol-2-yl)prop-2-yn-1-yl]amino}benzene-1-sulfonamide |
| 488. | 2-(dimethylamino)ethyl 2-{4-[(2-{3-[(4-methanesulfonylphenyl)-amino]prop-1-yn-1-yl}- |
| | 1-(2,2,2-trifluoroethyl)-1H-indol-4-yl)amino]piperidin-1-yl}acetate |
| 489. | 2-methoxy-4-[(3-{4-[(oxan-4-yl)amino]-1-(2,2,2-trifluoroethyl)-1H-indol-2-yl}prop-2- |
| | yn-1-yl)amino]benzene-1-sulfonamide |
| 490. | 2-chloro-4-[(3-{4-[(oxan-4-yl)amino]-1-(2,2,2-trifluoroethyl)-1H-indol-2-yl}prop-2-yn-1- |
| | yl)amino]benzene-1-sulfonamide |
| 491. | 3-methoxy-4-{[3-(4-{[1-(oxan-4-yl)piperidin-4-yl]amino}-1-(2,2,2-trifluoroethyl)-1H- |
| | indol-2-yl)prop-2-yn-1-yl]amino}benzene-1-sulfonamide |
| 492. | 4-{[3-(4-{[1-(2,3-dihydroxypropyl)-piperidin-4-yl]amino}-1-(2,2,2-trifluoro-ethyl)-1H- |
| | indol-2-yl)prop-2-yn-1-yl]amino}-3-methoxybenzene-1-sulfonamide |
| 493. | 3-methoxy-4-[(3-{4-[(piperidin-4-yl)amino]-1-(2,2,2-trifluoroethyl)-1H-indol-2-yl}prop- |
| | 2-yn-1-yl)amino]benzene-1-sulfonamide |
| 494. | 3-methoxy-4-[(3-{4-[(oxan-4-yl)amino]-1-(2,2,2-trifluoroethyl)-1H-indol-2-yl}prop-2- |
| | yn-1-yl)amino]benzene-1-sulfonamide |
| 495. | 3-methoxy-4-[(3-{4-[(oxan-4-yl)amino]-1-(2,2,2-trifluoroethyl)-1H-indol-2-yl}prop-2- |
| | yn-1-yl)amino]benzamide |
| 496. | 3-methoxy-4-{[3-(4-{[1-(oxan-4-yl)piperidin-4-yl]amino}-1-(2,2,2-trifluoroethyl)-1H- |
| | indol-2-yl)prop-2-yn-1-yl]amino}benzamide |
| 497. | 4-{[3-(4-{[1-(2,3-dihydroxypropyl)-piperidin-4-yl]amino}-1-(2,2,2-trifluoroethyl)-1H- |
| | indol-2-yl)prop-2-yn-1-yl]amino}-3-methoxybenzamide |
| 498. | 2-[5-({3-[1-(cyanomethyl)-4-[(1-methylpiperidin-4-yl)amino]-1H-indol-2-yl]prop-2-yn-1- |
| | yl}amino)pyridin-2-yl]-2-methylpropanenitrile |
| 499. | 2-[5-({3-[1-(3-methoxypropyl)-4-[(1-methylpiperidin-4-yl)amino]-1H-indol-2-yl]prop-2- |
| | yn-1-yl}amino)pyridin-2-yl]-2-methylpropanenitrile |
| 500. | 2-[5-({3-[1-(2-chloroethyl)-4-[(1-methylpiperidin-4-yl)amino]-1H-indol-2-yl]prop-2-yn- |
| | 1-yl}amino)pyridin-2-yl]-2-methylpropanenitrile |
| 501. | 2-methyl-2-{5-[(3-{4-[(1-methylpiperidin-4-yl)amino]-1-(propan-2-yl)-1H-indol-2- |
| | yl}prop-2-yn-1-yl)amino]pyridin-2-yl}propanenitrile |
| 502. | 2-{5-[(3-{1-cyclopentyl-4-[(1-methylpiperidin-4-yl)amino]-1H-indol-2-yl}prop-2-yn-1- |
| | yl)amino]pyridin-2-yl}-2-methylpropanenitrile |
| 503. | 2-methyl-2-{5-[(3-{4-[(1-methylpiperidin-4-yl)amino]-1-(3,3,3-trifluoropropyl)-1H- |
| | indol-2-yl}prop-2-yn-1-yl)amino]pyridin-2-yl}propanenitrile |
| 504. | 1-(2-chloroethyl)-N-(1-methylpiperidin-4-yl)-2-{3-[(6-methylpyridin-3-yl)amino]-prop-1- |
| | yn-1-yl}-1H-indol-4-amine |
| 505. | 1-(2-chloroethyl)-N-(1-methylpiperidin-4-yl)-2-{3-[(6-methylpyridin-3-yl)amino]-prop-1- |
| | yn-1-yl}-1H-indol-4-amine |
| 506. | 1-(2-chloroethyl)-2-{3-[(4-chlorophenyl)-amino]prop-1-yn-1-yl}-N-(1-methylpiperidin-4- |
| | yl)-1H-indol-4-amine |
| 507. | 2-[5-({3-[1-(1-cyanoethyl)-4-[(1-methylpiperidin-4-yl)amino]-1H-indol-2-yl]prop-2-yn- |
| | 1-yl}amino)pyridin-2-yl]-2-methylpropanenitrile |
| 508. | 2-[5-({3-[1-(cyanomethyl)-4-[(1,1-dioxo-1λ⁶-thian-4-yl)amino]-1H-indol-2-yl]prop-2-yn- |
| | 1-yl}amino)pyridin-2-yl]-2-methylpropanenitrile |
| 509. | 4-[(2-{3-[(4-methanesulfonylphenyl)amino]prop-1-yn-1-yl}-1-(oxiran-2-ylmethyl)-1H- |
| | indol-4-yl)amino]-1λ⁶-thiane-1,1-dione |
| 510. | 2-{3-[(4-methanesulfonylphenyl)-amino]prop-1-yn-1-yl}-N-[1-(oxan-4-yl)piperidin-4- |
| | yl]-1-(oxiran-2-ylmethyl)-1H-indol-4-amine |
| 511. | 4-[(2-{3-[(4-methanesulfonyl-2-methoxyphenyl)amino]prop-1-yn-1-yl}-1-(oxiran-2- |
| | ylmethyl)-1H-indol-4-yl)amino]-1λ⁶-thiane-1,1-dione |
| 512. | 2-{3-[(4-methanesulfonyl-2-methoxy-phenyl)amino]prop-1-yn-1-yl}-N-[1-(oxan-4- |
| | yl)piperidin-4-yl]-1-(oxiran-2-ylmethyl)-1H-indol-4-amine |
| 513. | 1-[6-(1-cyano-1-methylethyl)pyridin-3-yl]-3-(3-{4-[(1-methylpiperidin-4-yl)amino]-1- |
| | (2,2,2-trifluoroethyl)-1H-indol-2-yl}prop-2-yn-1-yl)urea |
| 514. | 1-(6-methanesulfonylpyridin-3-yl)-3-(3-{4-[(1-methylpiperidin-4-yl)amino]-1-(2,2,2- |
| | trifluoroethyl)-1H-indol-2-yl}prop-2-yn-1-yl)urea |
| 515. | 1-[6-(1-cyano-1-methylethyl)pyridin-3-yl]-3-(3-{4-[(1,1-dioxo-1λ⁶-thian-4-yl)amino]-1- |
| | (2,2,2-trifluoroethyl)-1H-indol-2-yl}prop-2-yn-1-yl)urea |
| 516. | 3-(3-{4-[(1,1-dioxo-1λ⁶-thian-4-yl)amino]-1-(2,2,2-trifluoroethyl)-1H-indol-2-yl}prop-2- |
| | yn-1-yl)-1-(6-methanesulfonylpyridin-3-yl)urea |
| 517. | 1-(6-cyanopyridin-3-yl)-3-(3-{4-[(1-methylpiperidin-4-yl)amino]-1-(2,2,2-trifluoroethyl)- |
| | 1H-indol-2-yl}prop-2-yn-1-yl)urea |
| 518. | 1-(6-cyanopyridin-3-yl)-3-(3-{4-[(1,1-dioxo-1λ⁶-thian-4-yl)amino]-1-(2,2,2- |
| | trifluoroethyl)-1H-indol-2-yl}prop-2-yn-1-yl)urea |
| 519. | 3-(3-{4-[(1,1-dioxo-1λ⁶-thian-4-yl)amino]-1-(2,2,2-trifluoroethyl)-1H-indol-2-yl}prop-2- |
| | yn-1-yl)-1-(quinoxalin-6-yl)urea |
| 520. | N-(3-{4-[(1,1-dioxo-1λ⁶-thian-4-yl)amino]-1-(2,2,2-trifluoroethyl)-1H-indol-2-yl}prop-2- |
| | yn-1-yl)-4-methylpiperazine-1-carboxamide |
| 521. | N-(3-{4-[(1,1-dioxo-1λ⁶-thian-4-yl)amino]-1-(2,2,2-trifluoroethyl)-1H-indol-2-yl}prop-2- |
| | yn-1-yl)morpholine-4-carboxamide |
| 522. | 4-[(2-{3-[(6-methanesulfonylpyridin-3-yl)amino]prop-1-yn-1-yl}-1-(2,2,2-trifluoroethyl)- |
| | 1H-indol-4-yl)amino]-1λ⁶-thiane-1,1-dione |
| 523. | 2-{4-[(2-{3-[(6-methanesulfonylpyridin-3-yl)amino]prop-1-yn-1-yl}-1-(2,2,2- |
| | trifluoroethyl)-1H-indol-4-yl)amino]piperidin-1-yl}-N,N-dimethylacetamide |
| 524. | N-(1-ethylpiperidin-4-yl)-2-{3-[(6-methanesulfonylpyridin-3-yl)amino]prop-1-yn-1-yl}- |
| | 1-(2,2,2-trifluoroethyl)-1H-indol-4-amine |
| 525. | N-[1-(2-methanesulfonylethyl)piperidin-4-yl]-2-{3-[(6-methanesulfonylpyridin-3- |
| | yl)amino]prop-1-yn-1-yl}-1-(2,2,2-trifluoroethyl)-1H-indol-4-amine |
| 526. | 2-{3-[(6-methanesulfonylpyridin-3-yl)amino]prop-1-yn-1-yl}-N-[1-(1-methylpiperidin-4- |
| | yl)piperidin-4-yl]-1-(2,2,2-trifluoroethyl)-1H-indol-4-amine |
| 527. | 2-{4-[(2-{3-[(6-methanesulfonylpyridin-3-yl)amino]prop-1-yn-1-yl}-1-(2,2,2- |
| | trifluoroethyl)-1H-indol-4-yl)amino]piperidin-1-yl}ethan-1-ol |
| 528. | 4-{4-[(2-{3-[(6-methanesulfonylpyridin-3-yl)amino]prop-1-yn-1-yl}-1-(2,2,2- |
| | trifluoroethyl)-1H-indol-4-yl)amino]piperidin-1-yl}-1λ⁶-thiane-1,1-dione |
| 529. | 2-{4-[(2-{3-[(6-methanesulfonylpyridin-3-yl)amino]prop-1-yn-1-yl}-1-(2,2,2- |
| | trifluoroethyl)-1H-indol-4-yl)amino]piperidin-1-yl}-1-(4-methylpiperazin-1-yl)ethan-1- |
| | one |
| 530. | 2-{3-[(4-methanesulfonylphenyl)amino]prop-1-yn-1-yl}-N-(oxan-4-yl)-1-(2,2,2- |
| | trifluoroethyl)-1H-indol-4-amine |
| 531. | 2-{4-[(2-{3-[(4-methanesulfonylphenyl)amino]prop-1-yn-1-yl}-1-(2,2,2-trifluoroethyl)- |
| | 1H-indol-4-yl)amino]piperidin-1-yl}ethan-1-ol |
| 532. | 2-{4-[(2-{3-[(4-methanesulfonylphenyl)amino]prop-1-yn-1-yl}-1-(2,2,2-trifluoroethyl)- |
| | 1H-indol-4-yl)amino]piperidin-1-yl}-1-(4-methylpiperazin-1-yl)ethan-1-one |
| 533. | 2-{3-[(4-methanesulfonylphenyl)amino]prop-1-yn-1-yl}-N-[1-(1-methylpiperidin-4- |
| | yl)piperidin-4-yl]-1-(2,2,2-trifluoroethyl)-1H-indol-4-amine |
| 534. | N-(2,3-dihydroxypropyl)-2-{4-[(2-{3-[(4-methanesulfonylphenyl)-amino]prop-1-yn-1- |
| | yl}-1-(2,2,2-trifluoroethyl)-1H-indol-4-yl)amino]piperidin-1-yl}-N-methylacetamide |
| 535. | 4-N-(2-{3-[(4-methanesulfonylphenyl)-amino]prop-1-yn-1-yl}-1-(2,2,2-trifluoroethyl)- |
| | 1H-indol-4-yl)-1-N,1-N-dimethylcyclohexane-1,4-diamine |
| 536. | (1S,4S)-4-N-(2-{3-[(4-methanesulfonyl-phenyl)amino]prop-1-yn-1-yl}-1-(2,2,2- |
| | trifluoroethyl)-1H-indol-4-yl)-1-N,1-N-dimethylcyclohexane-1,4-diamine |
| 537. | 2-{3-[(4-methanesulfonylphenyl)-amino]prop-1-yn-1-yl}-N-[1-(oxan-4-yl)piperidin-4- |
| | yl]-1-(2,2,2-trifluoroethyl)-1H-indol-4-amine |
| 538. | 2-{4-[(2-{3-[(4-methanesulfonylphenyl)amino]prop-1-yn-1-yl}-1-(2,2,2-trifluoroethyl)- |
| | 1H-indol-4-yl)amino]piperidin-1-yl}-1-(morpholin-4-yl)ethan-1-one |
| 539. | 1-(4-hydroxypiperidin-1-yl)-2-{4-[(2-{3-[(4-methanesulfonylphenyl)-amino]prop-1-yn-1- |
| | yl}-1-(2,2,2-trifluoroethyl)-1H-indol-4-yl)amino]piperidin-1-yl}ethan-1-one |
| 540. | 2-{3-[(4-methanesulfonylphenyl)-amino]prop-1-yn-1-yl}-N-(piperidin-4-yl)-1-(2,2,2- |
| | trifluoroethyl)-1H-indol-4-amine |
| 541. | N-{1-[1-(2-methanesulfonylethyl)piperidin-4-yl]piperidin-4-yl}-2-{3-[(4- |
| | methanesulfonylphenyl)amino]prop-1-yn-1-yl}-1-(2,2,2-trifluoroethyl)-1H-indol-4-amine |
| 542. | 2-{3-[(4-methanesulfonylphenyl)-amino]prop-1-yn-1-yl}-N-[1-(2- |
| | methoxyethyl)piperidin-4-yl]-1-(2,2,2-trifluoroethyl)-1H-indol-4-amine |
| 543. | 3-{4-[(2-{3-[(4-methane-sulfonylphenyl)-amino]prop-1-yn-1-yl}-1-(2,2,2-trifluoroethyl)- |
| | 1H-indol-4-yl)amino]piperidin-1-yl}propanenitrile |
| 544. | 2-{3-[(4-methanesulfonylphenyl)-amino]prop-1-yn-1-yl}-N-(1-methylpiperidin-4-yl)-1- |
| | (2,2,2-trifluoroethyl)-1H-indol-4-amine |
| 545. | 2-{4-[(2-{3-[(4-methane-sulfonylphenyl)-amino]prop-1-yn-1-yl}-1-(2,2,2-trifluoroethyl)- |
| | 1H-indol-4-yl)amino]piperidin-1-yl}acetamide |
| 546. | 4-{4-[(2-{3-[(4-methane-sulfonylphenyl)-amino]prop-1-yn-1-yl}-1-(2,2,2-trifluoroethyl)- |
| | 1H-indol-4-yl)amino]piperidin-1-yl}-1λ⁶-thiane-1,1-dione |
| 547. | 2-{4-[(2-{3-[(4-methane-sulfonylphenyl)amino]prop-1-yn-1-yl}-1-(2,2,2-trifluoroethyl)- |
| | 1H-indol-4-yl)amino]piperidin-1-yl}-N-methylacetamide |
| 548. | 2-(3-{[4-(ethanesulfonyl)phenyl]amino}prop-1-yn-1-yl)-N-(1-methylpiperidin-4-yl)-1- |
| | (2,2,2-trifluoroethyl)-1H-indol-4-amine |
| 549. | 2-{4-[(2-{3-[(2-fluoro-4-methanesulfonylphenyl)amino]prop-1-yn-1-yl}-1-(2,2,2- |
| | trifluoroethyl)-1H-indol-4-yl)amino]piperidin-1-yl}ethan-1-ol |
| 550. | 2-{3-[(2-fluoro-4-methane-sulfonylphenyl)-amino]prop-1-yn-1-yl}-N-[1-(2- |
| | methanesulfonylethyl)-piperidin-4-yl]-1-(2,2,2-trifluoroethyl)-1H-indol-4-amine |
| 551. | 2-{4-[(2-{3-[(2-fluoro-4-methanesulfonylphenyl)amino]prop-1-yn-1-yl}-1-(2,2,2- |
| | trifluoroethyl)-1H-indol-4-yl)amino]piperidin-1-yl}acetamide |
| 552. | 2-{3-[(2-fluoro-4-methane-sulfonylphenyl)-amino]prop-1-yn-1-yl}-N-(1-methylpiperidin- |
| | 4-yl)-1-(2,2,2-trifluoroethyl)-1H-indol-4-amine |
| 553. | 1-{4-[(2-{3-[(4-methanesulfonyl-phenyl)amino]prop-1-yn-1-yl}-1-(2,2,2-trifluoroethyl)- |
| | 1H-indol-4-yl)amino]piperidin-1-yl}-2-methoxyethan-1-one |
| 554. | 2-{3-[(4-methanesulfonyl-phenyl)amino]prop-1-yn-1-yl}-N-(1-methylpyrrolidin-3-yl)-1- |
| | (2,2,2-trifluoroethyl)-1H-indol-4-amine |
| 555. | N-hydroxy-2-{4-[(2-{3-[(4-methanesulfonylphenyl)amino]prop-1-yn-1-yl}-1-(2,2,2- |
| | trifluoroethyl)-1H-indol-4-yl)amino]piperidin-1-yl}acetamide |
| 556. | 3-{4-[(2-{3-[(4-methanesulfonyl-phenyl)amino]prop-1-yn-1-yl}-1-(2,2,2-trifluoroethyl)- |
| | 1H-indol-4-yl)amino]piperidin-1-yl}propane-1,2-diol |
| 557. | 2-{3-[(2-fluoro-4-methanesulfonyl-phenyl)amino]prop-1-yn-1-yl}-N-[1-(1- |
| | methylpiperidin-4-yl)piperidin-4-yl]-1-(2,2,2-trifluoroethyl)-1H-indol-4-amine |
| 558. | 2-(3-{[4-(ethanesulfonyl)phenyl]amino}prop-1-yn-1-yl)-N-[1-(1-methylpiperidin-4- |
| | yl)piperidin-4-yl]-1-(2,2,2-trifluoroethyl)-1H-indol-4-amine |
| 559. | 2-{3-[(2-fluoro-4-methanesulfonyl-phenyl)amino]prop-1-yn-1-yl}-N-(oxan-4-yl)-1- |
| | (2,2,2-trifluoroethyl)-1H-indol-4-amine |
| 560. | 2-(4-{[2-(3-{[4-(ethanesulfonyl)-phenyl]amino}prop-1-yn-1-yl)-1-(2,2,2-trifluoroethyl)- |
| | 1H-indol-4-yl]amino}piperidin-1-yl)ethan-1-ol |
| 561. | 1-{4-[(2-{3-[(4-methanesulfonyl-phenyl)amino]prop-1-yn-1-yl}-1-(2,2,2-trifluoroethyl)- |
| | 1H-indol-4-yl)amino]piperidin-1-yl}propan-2-ol |
| 562. | 2-(3-{[4-(ethanesulfonyl)-phenyl]amino}prop-1-yn-1-yl)-N-[1-(2- |
| | methanesulfonylethyl)piperidin-4-yl]-1-(2,2,2-trifluoroethyl)-1H-indol-4-amine |
| 563. | 4-{[1-(2,2,2-trifluoroethyl)-2-{3-[(4-trifluoromethanesulfonylphenyl)amino]prop-1-yn-1- |
| | yl}-1H-indol-4-yl]amino}-1λ⁶-thiane-1,1-dione |
| 564. | 2-(4-{[1-(2,2,2-trifluoroethyl)-2-{3-[(4-trifluoromethanesulfonylphenyl)amino]prop-1-yn- |
| | 1-yl}-1H-indol-4-yl]amino}piperidin-1-yl)ethan-1-ol |
| 565. | N-(1-methylpiperidin-4-yl)-1-(2,2,2-trifluoroethyl)-2-{3-[(4- |
| | trifluoromethanesulfonylphenyl)amino]prop-1-yn-1-yl}-1H-indol-4-amine |
| 566. | 2-{3-[(4-methanesulfonyl-phenyl)amino]prop-1-yn-1-yl}-N-[1-(1-methylpyrrolidin-3- |
| | yl)piperidin-4-yl]-1-(2,2,2-trifluoroethyl)-1H-indol-4-amine |
| 567. | 2-{3-[(4-methanesulfonyl-phenyl)amino]prop-1-yn-1-yl}-N-[1-(3- |
| | methanesulfonylpropyl)piperidin-4-yl]-1-(2,2,2-trifluoroethyl)-1H-indol-4-amine |
| 568. | 2-(3-{[4-(ethanesulfonyl)-phenyl]amino}prop-1-yn-1-yl)-N-[1-(2- |
| | methoxyethyl)piperidin-4-yl]-1-(2,2,2-trifluoroethyl)-1H-indol-4-amine |
| 569. | 4-(4-{[2-(3-{[4-(ethanesulfonyl)-phenyl]amino}prop-1-yn-1-yl)-1-(2,2,2-trifluoroethyl)- |
| | 1H-indol-4-yl]amino}piperidin-1-yl)-1λ⁶-thiane-1,1-dione |
| 570. | 2-(3-{[4-(ethanesulfonyl)-phenyl]amino}prop-1-yn-1-yl)-N-[1-(oxan-4-yl)piperidin-4-yl]- |
| | 1-(2,2,2-trifluoroethyl)-1H-indol-4-amine |
| 571. | N-[1-(2-methanesulfonylethyl)-piperidin-4-yl]-1-(2,2,2-trifluoroethyl)-2-{3-[(4- |
| | trifluoromethane-sulfonylphenyl)amino]prop-1-yn-1-yl}-1H-indol-4-amine |
| 572. | N-[1-(oxan-4-yl)piperidin-4-yl]-1-(2,2,2-trifluoroethyl)-2-{3-[(4- |
| | trifluoromethanesulfonylphenyl)amino]prop-1-yn-1-yl}-1H-indol-4-amine |
| 573. | N-[1-(1-methylpiperidin-4-yl)piperidin-4-yl]-1-(2,2,2-trifluoroethyl)-2-{3-[(4- |
| | trifluoromethanesulfonylphenyl)amino]prop-1-yn-1-yl}-1H-indol-4-amine |
| 574. | 2-{3-[(2-fluoro-4-methanesulfonyl-phenyl)amino]prop-1-yn-1-yl}-N-[1-(oxan-4- |
| | yl)piperidin-4-yl]-1-(2,2,2-trifluoroethyl)-1H-indol-4-amine |
| 575. | 2-{4-[(2-{3-[(4-methanesulfonyl-phenyl)amino]prop-1-yn-1-yl}-1-(2,2,2-trifluoroethyl)- |
| | 1H-indol-4-yl)amino]piperidin-1-yl}acetonitrile |
| 576. | 2-{3-[(2-fluoro-4-methanesulfonylphenyl)amino]prop-1-yn-1-yl}-N-[1-(2- |
| | methoxyethyl)piperidin-4-yl]-1-(2,2,2-trifluoroethyl)-1H-indol-4-amine |
| 577. | 2-{3-[(3-chloro-4-methanesulfonylphenyl)amino]prop-1-yn-1-yl}-N-[1-(oxan-4- |
| | yl)piperidin-4-yl]-1-(2,2,2-trifluoroethyl)-1H-indol-4-amine |
| 578. | 2-{3-[(2,6-difluoro-4-methanesulfonyl-phenyl)amino]prop-1-yn-1-yl}-N-[1-(oxan-4- |
| | yl)piperidin-4-yl]-1-(2,2,2-trifluoroethyl)-1H-indol-4-amine |
| 579. | 2-{3-[(3-chloro-4-methanesulfonyl-phenyl)amino]prop-1-yn-1-yl}-N-[1-(2- |
| | methoxyethyl)piperidin-4-yl]-1-(2,2,2-trifluoroethyl)-1H-indol-4-amine |
| 580. | 2-{4-[(2-{3-[(3-chloro-4-methanesulfonylphenyl)amino]prop-1-yn-1-yl}-1-(2,2,2- |
| | trifluoroethyl)-1H-indol-4-yl)amino]piperidin-1-yl}ethan-1-ol |
| 581. | (2S)-3-{4-[(2-{3-[(4-methanesulfonyl-phenyl)amino]prop-1-yn-1-yl}-1-(2,2,2- |
| | trifluoroethyl)-1H-indol-4-yl)amino]piperidin-1-yl}propane-1,2-diol |
| 582. | N-(5-aminopentyl)-2-{4-[(2-{3-[(4-methanesulfonylphenyl)amino]prop-1-yn-1-yl}-1- |
| | (2,2,2-trifluoroethyl)-1H-indol-4-yl)amino]piperidin-1-yl}acetamide |
| 583. | 2-{3-[(2,6-difluoro-4-methanesulfonyl-phenyl)amino]prop-1-yn-1-yl}-N-[1-(2- |
| | methoxyethyl)piperidin-4-yl]-1-(2,2,2-trifluoroethyl)-1H-indol-4-amine |
| 584. | 2-(3-{[4-(ethanesulfonyl)phenyl]-amino}prop-1-yn-1-yl)-N-(oxan-4-yl)-1-(2,2,2- |
| | trifluoroethyl)-1H-indol-4-amine |
| 585. | 2-(4-{[2-(3-{[4-(ethanesulfonyl)phenyl]amino}prop-1-yn-1-yl)-1-(2,2,2-trifluoroethyl)- |
| | 1H-indol-4-yl]amino}piperidin-1-yl)acetonitrile |
| 586. | 2-(3-{[4-(2-methylpropane-2-sulfonyl)phenyl]amino}prop-1-yn-1-yl)-N-[1-(oxan-4- |
| | yl)piperidin-4-yl]-1-(2,2,2-trifluoroethyl)-1H-indol-4-amine |
| 587. | 2-(2-{4-[(2-{3-[(4-methanesulfonyl-phenyl)amino]prop-1-yn-1-yl}-1-(2,2,2- |
| | trifluoroethyl)-1H-indol-4-yl)amino]piperidin-1-yl}ethoxy)ethan-1-ol |
| 588. | 1-{4-[(2-{3-[(2-fluoro-4-methane-sulfonylphenyl)amino]prop-1-yn-1-yl}-1-(2,2,2- |
| | trifluoroethyl)-1H-indol-4-yl)amino]piperidin-1-yl}propan-2-ol |
| 589. | 3-{4-[(2-{3-[(2-fluoro-4-methanesulfonylphenyl)amino]prop-1-yn-1-yl}-1-(2,2,2- |
| | trifluoroethyl)-1H-indol-4-yl)amino]piperidin-1-yl}propane-1,2-diol |
| 590. | (1S,4S)-4-N-(2-{3-[(2-fluoro-4-methanesulfonylphenyl)amino]prop-1-yn-1-yl}-1-(2,2,2- |
| | trifluoroethyl)-1H-indol-4-yl)-1-N,1-N-dimethylcyclohexane-1,4-diamine |
| 591. | 3-(4-{[2-(3-{[4-(ethanesulfonyl)-phenyl]amino}prop-1-yn-1-yl)-1-(2,2,2-trifluoroethyl)- |
| | 1H-indol-4-yl]amino}piperidin-1-yl)propane-1,2-diol |
| 592. | 2-(3-{[4-(ethanesulfonyl)-phenyl]amino}prop-1-yn-1-yl)-N-[1-(3- |
| | methanesulfonylpropyl)-piperidin-4-yl]-1-(2,2,2-trifluoroethyl)-1H-indol-4-amine |
| 593. | 1-(4-{[2-(3-{[4-(ethanesulfonyl)-phenyl]amino}prop-1-yn-1-yl)-1-(2,2,2-trifluoroethyl)- |
| | 1H-indol-4-yl]amino}piperidin-1-yl)propan-2-ol |
| 594. | 2-[2-(4-{[2-(3-{[4-(ethanesulfonyl)-phenyl]amino}prop-1-yn-1-yl)-1-(2,2,2- |
| | trifluoroethyl)-1H-indol-4-yl]amino}piperidin-1-yl)ethoxy]ethan-1-ol |
| 595. | (1R,4R)-4-N-(2-{3-[(2-fluoro-4-methanesulfonylphenyl)amino]prop-1-yn-1-yl}-1-(2,2,2- |
| | trifluoroethyl)-1H-indol-4-yl)-1-N,1-N-dimethyl-cyclohexane-1,4-diamine |
| 596. | 2-{3-[(2,6-difluoro-4-methanesulfonylphenyl)amino]prop-1-yn-1-yl}-N-[1-(2- |
| | methanesulfonylethyl)piperidin-4-yl]-1-(2,2,2-trifluoroethyl)-1H-indol-4-amine |
| 597. | 4-{4-[(2-{3-[(2,6-difluoro-4-methanesulfonylphenyl)amino]prop-1-yn-1-yl}-1-(2,2,2- |
| | trifluoroethyl)-1H-indol-4-yl)amino]piperidin-1-yl}-1λ⁶⁻thiane-1,1-dione |
| 598. | 2-{3-[(4-methanesulfonyl-3-methoxyphenyl)amino]prop-1-yn-1-yl}-N-[1-(oxan-4- |
| | yl)piperidin-4-yl]-1-(2,2,2-trifluoroethyl)-1H-indol-4-amine |
| 599. | 2-{3-[(4-methanesulfonyl-3-methoxyphenyl)amino]prop-1-yn-1-yl}-N-[1-(2- |
| | methoxyethyl)piperidin-4-yl]-1-(2,2,2-trifluoroethyl)-1H-indol-4-amine |
| 600. | 4-N-(2-{3-[(2-fluoro-4-methanesulfonylphenyl)amino]prop-1-yn-1-yl}-1-(2,2,2- |
| | trifluoroethyl)-1H-indol-4-yl)-1-N,1-N-dimethylcyclohexane-1,4-diamine |
| 601. | 2-{4-[(2-{3-[(4-methanesulfonyl-phenyl)amino]prop-1-yn-1-yl}-1-(2,2,2-trifluoroethyl)- |
| | 1H-indol-4-yl)amino]piperidin-1-yl}acetic acid |
| 602. | 2-hydroxyethyl 2-{4-[(2-{3-[(4-methanesulfonylphenyl)amino]prop-1-yn-1-yl}-1-(2,2,2- |
| | trifluoroethyl)-1H-indol-4-yl)amino]piperidin-1-yl}acetate |
| 603. | 2-{3-[(4-methanesulfonylphenyl)-amino]prop-1-yn-1-yl}-N-(2-methylpiperidin-4-yl)-1- |
| | (2,2,2-trifluoroethyl)-1H-indol-4-amine |
| 604. | 2-{4-[(2-{3-[(4-methanesulfonyl-3-methoxyphenyl)amino]prop-1-yn-1-yl}-1-(2,2,2- |
| | trifluoroethyl)-1H-indol-4-yl)amino]piperidin-1-yl}ethan-1-ol |
| 605. | (2S)-2-(2-{4-[(2-{3-[(4-methane-sulfonylphenyl)amino]prop-1-yn-1-yl}-1-(2,2,2- |
| | trifluoroethyl)-1H-indol-4-yl)amino]piperidin-1-yl}acetamido)pentanedioic acid |
| 606. | 1,5-dimethyl (2S)-2-(2-{4-[(2-{3-[(4-methanesulfonylphenyl)amino]prop-1-yn-1-yl}-1- |
| | (2,2,2-trifluoroethyl)-1H-indol-4-yl)amino]piperidin-1-yl}acetamido)pentanedioate |
| 607. | N-(4-carbamimidamidobutyl)-2-{4-[(2-{3-[(4-methanesulfonylphenyl)-amino]prop-1-yn- |
| | 1-yl}-1-(2,2,2-trifluoroethyl)-1H-indol-4-yl)amino]piperidin-1-yl}acetamide |
| 608. | 2-{3-[(4-methanesulfonyl-2-methoxyphenyl)amino]prop-1-yn-1-yl}-N-(oxan-4-yl)-1- |
| | (2,2,2-trifluoroethyl)-1H-indol-4-amine |
| 609. | 2-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}ethan-1-ol |
| 610. | 2-{3-[(5-methanesulfonylpyridin-2-yl)amino]prop-1-yn-1-yl}-N-(oxan-4-yl)-1-(2,2,2- |
| | trifluoroethyl)-1H-indol-4-amine |
| 611. | 3-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}propane-1,2-diol |
| 612. | 4-[(2-{3-[(2,4-dimethoxyphenyl)-amino]prop-1-yn-1-yl}-1-(2,2,2-trifluoroethyl)-1H- |
| | indol-4-yl)amino]-1λ⁶-thiane-1,1-dione |
| 613. | methyl 4-[(3-{4-[(1,1-dioxo-1λ⁶-thian-4-yl)amino]-1-(2,2,2-trifluoroethyl)-1H-indol-2- |
| | yl}prop-2-yn-1-yl)amino]-3-methoxybenzoate |
| 614. | 2-{3-[(4-methanesulfonyl-2-methoxyphenyl)amino]prop-1-yn-1-yl}-N-[1-(2- |
| | methanesulfonylethyl)-piperidin-4-yl]-1-(2,2,2-trifluoroethyl)-1H-indol-4-amine |
| 615. | 2-{3-[(4-methanesulfonyl-2-methoxyphenyl)amino]prop-1-yn-1-yl}-N-[1-(2- |
| | methoxyethyl)piperidin-4-yl]-1-(2,2,2-trifluoroethyl)-1H-indol-4-amine |
| 616. | (1S,4S)-4-N-(2-{3-[(4-methanesulfonyl-2-methoxyphenyl)amino]prop-1-yn-1-yl}-1- |
| | (2,2,2-trifluoroethyl)-1H-indol-4-yl)-1-N,1-N-dimethylcyclohexane-1,4-diamine |
| 617. | (1R,4R)-4-N-(2-{3-[(4-methanesulfonyl-2-methoxyphenyl)amino]prop-1-yn-1-yl}-1- |
| | (2,2,2-trifluoroethyl)-1H-indol-4-yl)-1-N,1-N-dimethylcyclohexane-1,4-diamine |
| 618. | 2-{3-[(4-methanesulfonyl-2-methoxyphenyl)amino]prop-1-yn-1-yl}-N-[1-(oxan-4- |
| | yl)piperidin-4-yl]-1-(2,2,2-trifluoroethyl)-1H-indol-4-amine |
| 619. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}propan-2-o |
| 620. | 2-{3-[(4-methanesulfonyl-2-methoxyphenyl)amino]prop-1-yn-1-yl}-N-[1-(1- |
| | methylpyrrolidin-3-yl)piperidin-4-yl]-1-(2,2,2-trifluoroethyl)-1H-indol-4-amine |
| 621. | 2-{3-[(4-methanesulfonyl-2-methoxyphenyl)amino]prop-1-yn-1-yl}-N-(2- |
| | methylpiperidin-4-yl)-1-(2,2,2-trifluoroethyl)-1H-indol-4-amine |
| 622. | 2-{3-[(4-methanesulfonyl-2-methoxyphenyl)amino]prop-1-yn-1-yl}-N-(piperidin-4-yl)-1- |
| | (2,2,2-trifluoroethyl)-1H-indol-4-amine |
| 623. | 2-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-1-(4-methylpiperazin-1-yl)ethan-1-one |
| 624. | 2-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}acetamide |
| 625. | 2-{3-[(4-methanesulfonyl-2-methoxyphenyl)amino]prop-1-yn-1-yl}-N-[1-(1- |
| | methylpiperidin-4-yl)piperidin-4-yl]-1-(2,2,2-trifluoroethyl)-1H-indol-4-amine |
| 626. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]cyclohexyl}piperidin-4-ol |
| 627. | 2-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-1-(morpholin-4-yl)ethan-1-one |
| 628. | 2-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-N,N-dimethylacetamide |
| 629. | 2-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}acetonitrile |
| 630. | methyl 2-{4-[(2-{3-[(4-methane-sulfonyl-2-methoxyphenyl)amino]prop-1-yn-1-yl}-1- |
| | (2,2,2-trifluoroethyl)-1H-indol-4-yl)amino]piperidin-1-yl}acetate |
| 631. | 1-(4-hydroxypiperidin-1-yl)-2-{4-[(2-{3-[(4-methanesulfonyl-2-methoxy- |
| | phenyl)amino]prop-1-yn-1-yl}-1-(2,2,2-trifluoroethyl)-1H-indol-4-yl)amino]piperidin-1- |
| | yl}ethan-1-one |
| 632. | 2-(2-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}ethoxy)ethan-1-ol |
| 633. | -[(1R,4R)-4-[(2-{3-[(4-methane-sulfonyl-2-methoxyphenyl)amino]prop-1-yn-1-yl}-1- |
| | (2,2,2-trifluoroethyl)-1H-indol-4-yl)amino]cyclohexyl]piperidin-4-ol |
| 634. | 2-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}acetic acid |
| 635. | (1R,4R)-4-[(2-{3-[(4-methanesulfonyl-2-methoxyphenyl)amino]prop-1-yn-1-yl}-1-(2,2,2- |
| | trifluoroethyl)-1H-indol-4-yl)amino]cyclohexan-1-ol |
| 636. | (1S,4S)-4-[(2-{3-[(4-methanesulfonyl-2-methoxyphenyl)amino]prop-1-yn-1-yl}-1-(2,2,2- |
| | trifluoroethyl)-1H-indol-4-yl)amino]cyclohexan-1-ol |
| 637. | 2-{3-[(4-methanesulfonyl-2-methoxyphenyl)amino]prop-1-yn-1-yl}-1-(2,2,2- |
| | trifluoroethyl)-1H-indol-4-amine |
| 638. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]cyclohexyl}-3-methylpyrrolidin-3-ol |
| 639. | (3R,4R)-1-{4-[(2-{3-[(4-methane-sulfonyl-2-methoxyphenyl)amino]prop-1-yn-1-yl}-1- |
| | (2,2,2-trifluoroethyl)-1H-indol-4-yl)amino]cyclohexyl}-pyrrolidine-3,4-diol |
| 640. | 4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidine-1-carboximidamide |
| 641. | 1-[(1S,4S)-4-[(2-{3-[(4-methane-sulfonyl-2-methoxyphenyl)amino]prop-1-yn-1-yl}-1- |
| | (2,2,2-trifluoroethyl)-1H-indol-4-yl)amino]cyclohexyl]piperidin-4-ol |
| 642. | 4-[(2-{3-[(3-methoxypyridin-4-yl)amino]prop-1-yn-1-yl}-1-(2,2,2-trifluoroethyl)-1H- |
| | indol-4-yl)amino]-1λ⁶-thiane-1,1-dione |
| 643. | 2-{3-[(4-methanesulfonyl-2-methoxyphenyl)amino]prop-1-yn-1-yl}-N-[(1S,4S)-4- |
| | (morpholin-4-yl)cyclohexyl]-1-(2,2,2-trifluoroethyl)-1H-indol-4-amine |
| 644. | 2-{3-[(4-methanesulfonyl-2-methoxyphenyl)amino]prop-1-yn-1-yl}-N-[(1R,4R)-4- |
| | (morpholin-4-yl)cyclohexyl]-1-(2,2,2-trifluoroethyl)-1H-indol-4-amine |
| 645. | 2-(3-{[2-(2-fluoroethoxy)-4-methanesulfonylphenyl]amino}prop-1-yn-1-yl)-N-[1-(oxan- |
| | 4-yl)piperidin-4-yl]-1-(2,2,2-trifluoroethyl)-1H-indol-4-amine |
| 646. | 4-{[2-(3-{[4-methanesulfonyl-2-(trifluoromethyl)phenyl]amino}prop-1-yn-1-yl)-1-(2,2,2- |
| | trifluoroethyl)-1H-indol-4-yl]amino}-1λ⁶-thiane-1,1-dione |
| 647. | 2-(3-{[4-methanesulfonyl-2-(trifluoromethyl)phenyl]amino}prop-1-yn-1-yl)-N-(1- |
| | methylpiperidin-4-yl)-1-(2,2,2-trifluoroethyl)-1H-indol-4-amine |
| 648. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}ethan-1-one |
| 649. | 3-methoxy-4-{[3-(4-{[1-(1-methylpiperidin-4-yl)piperidin-4-yl]amino}-1-(2,2,2- |
| | trifluoroethyl)-1H-indol-2-yl)prop-2-yn-1-yl]amino}benzene-1-sulfonamide |
| 650. | 3-methoxy-4-[(3-{4-[(piperidin-4-yl)amino]-1-(2,2,2-trifluoroethyl)-1H-indol-2-yl}prop- |
| | 2-yn-1-yl)amino]-benzamide |
| 651. | 3-methoxy-4-{[3-(4-{[1-(1-methylpiperidin-4-yl)piperidin-4-yl]amino}-1-(2,2,2- |
| | trifluoroethyl)-1H-indol-2-yl)prop-2-yn-1-yl]amino}benzamide |
| 652. | 3-(4-{[2-(3-{[2-(2-fluoroethoxy)-4-methanesulfonylphenyl]amino}prop-1-yn-1-yl)-1- |
| | (2,2,2-trifluoroethyl)-1H-indol-4-yl]amino}piperidin-1-yl)propane-1,2-diol |
| 653. | 3-(4-{[2-(3-{[4-methanesulfonyl-2-(2-methoxyethoxy)phenyl]amino}prop-1-yn-1-yl)-1- |
| | (2,2,2-trifluoroethyl)-1H-indol-4-yl]amino}piperidin-1-yl)propane-1,2-diol |
| 654. | 2-(4-{[2-(3-{[4-methanesulfonyl-2-(2-methoxyethoxy)phenyl]amino}prop-1-yn-1-yl)-1- |
| | (2,2,2-trifluoroethyl)-1H-indol-4-yl]amino}piperidin-1-yl)acetamide |
| 655. | 2-(4-{[2-(3-{[2-(2-fluoroethoxy)-4-methanesulfonylphenyl]amino}prop-1-yn-1-yl)-1- |
| | (2,2,2-trifluoroethyl)-1H-indol-4-yl]amino}piperidin-1-yl)-1-(4-methylpiperazin-1- |
| | yl)ethan-1-one |
| 656. | 2-(3-{[2-(2-fluoroethoxy)-4-methanesulfonylphenyl]amino}prop-1-yn-1-yl)-N-(oxan-4- |
| | yl)-1-(2,2,2-trifluoroethyl)-1H-indol-4-amine |
| 657. | 2-(3-{[2-(2-fluoroethoxy)-4-methanesulfonylphenyl]amino}prop-1-yn-1-yl)-N-[1-(1- |
| | methylpiperidin-4-yl)piperidin-4-yl]-1-(2,2,2-trifluoroethyl)-1H-indol-4-amine |
| 658. | 4-[(2-{3-[(4-methoxypyridin-3-yl)amino]prop-1-yn-1-yl}-1-(2,2,2-trifluoroethyl)-1H- |
| | indol-4-yl)amino]-1λ⁶-thiane-1,1-dione |
| 659. | S-{4-[(3-{4-[(1,1-dioxo-1λ⁶-thian-4-yl)amino]-1-(2,2,2-trifluoroethyl)-1H-indol-2- |
| | yl}prop-2-yn-1-yl)amino]-3-methoxyphenyl}-2-hydroxyethane-1-sulfonamido |
| 660. | 2-hydroxy-S-{3-methoxy-4-[(3-{4-[(1-methylpiperidin-4-yl)amino]-1-(2,2,2- |
| | trifluoroethyl)-1H-indol-2-yl}prop-2-yn-1-yl)amino]phenyl}ethane-1-sulfonamido |
| 661. | 2-methyl-2-[5-({3-[4-(morpholin-4-yl)-1-(2,2,2-trifluoroethyl)-1H-indol-2-yl]prop-2-yn- |
| | 1-yl}amino)pyridin-2-yl]propanenitrile |
| 662. | -{4-[(2-{3-[(4-methanesulfonylphenyl)amino]prop-1-yn-1-yl}-1-(2,2,2-trifluoroethyl)- |
| | 1H-indol-4-yl)amino]piperidin-1-yl}-N-[5-(2-{4-[(2-{3-[(4- |
| | methanesulfonylphenyl)amino]prop-1-yn-1-yl}-1-(2,2,2-trifluoroethyl)-1H-indol-4- |
| | yl)amino]piperidin-1-yl}acetamido)pentyl]acetamide |
| 663. | 6-[(2-{4-[(2-{3-[(4-methanesulfonylphenyl)amino]prop-1-yn-1-yl}-1-(2,2,2- |
| | trifluoroethyl)-1H-indol-4-yl)amino]piperidin-1-yl}acetyl)oxy]hexyl 2-{4-[(2-{3-[(4- |
| | methanesulfonylphenyl)amino]prop-1-yn-1-yl}-1-(2,2,2-trifluoroethyl)-1H-indol-4- |
| | yl)amino]piperidin-1-yl}acetate |
| 664. | 3-methoxy-4-({3-[4-({1-[2-(4-methylpiperazin-1-yl)-2-oxoethyl]piperidin-4-yl}amino)-1- |
| | (2,2,2-trifluoroethyl)-1H-indol-2-yl]prop-2-yn-1-yl}amino)benzene-1-sulfonamide |
| 665. | 2-{5-methanesulfonyl-2-[(3-{4-[(1-methylpiperidin-4-yl)amino]-1-(2,2,2-trifluoroethyl)- |
| | 1H-indol-2-yl}prop-2-yn-1-yl)amino]phenoxy}acetamide |
| 666. | 2-(3-{[4-methanesulfonyl-2-(2-methoxyethoxy)phenyl]amino}prop-1-yn-1-yl)-N-[l- |
| | (oxan-4-yl)piperidin-4-yl]-1-(2,2,2-trifluoroethyl)-1H-indol-4-amine |
| 667. | 2-(4-{[2-(3-{[4-methanesulfonyl-2-(2-methoxyethoxy)phenyl]amino}prop-1-yn-1-yl)-1- |
| | (2,2,2-trifluoroethyl)-1H-indol-4-yl]amino}piperidin-1-yl)ethan-1-ol |
| 668. | 3-methoxy-4-[(3-{4-[(oxan-4-yl)amino]-1-(2,2,2-trifluoroethyl)-1H-indol-2-yl}prop-2- |
| | yn-1-yl)amino]benzoic acid |
| 669. | 2-{2-[(3-{4-[(1,1-dioxo-1λ⁶-thian-4-yl)amino]-1-(2,2,2-trifluoroethyl)-1H-indol-2- |
| | yl}prop-2-yn-1-yl)amino]-5-methanesulfonylphenoxy}acetamide |
| 670. | 4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidine-1-carboxamide |
| 671. | 2-{3-[(4-methoxypyridin-3-yl)amino]prop-1-yn-1-yl}-N-(1-methylpiperidin-4-yl)-1- |
| | (2,2,2-trifluoroethyl)-1H-indol-4-amine |
| 672. | 4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidine-1-carbothioamide |
| 673. | 4-[(2-{3-[(6-methanesulfonyl-4-methoxypyridin-3-yl)amino]prop-1-yn-1-yl}-1-(2,2,2- |
| | trifluoroethyl)-1H-indol-4-yl)amino]-1λ⁶-thiane-1,1-dione |
| 674. | 3-methoxy-4-{[3-(4-{[1-(2-methoxyethyl)piperidin-4-yl]amino}-1-(2,2,2-trifluoroethyl)- |
| | 1H-indol-2-yl)prop-2-yn-1-yl]amino}benzene-1-sulfonamide |
| 675. | 4-{[2-(3-{[4-(ethanesulfonyl)-2-methoxyphenyl]amino}prop-1-yn-1-yl)-1-(2,2,2- |
| | trifluoroethyl)-1H-indol-4-yl]amino}-1λ⁶-thiane-1,1-dione |
| 676. | 2-(3-{[4-(ethanesulfonyl)-2-methoxyphenyl]amino}prop-1-yn-1-yl)-N-[1-(oxan-4- |
| | yl)piperidin-4-yl]-1-(2,2,2-trifluoroethyl)-1H-indol-4-amine |
| 677. | 4-{[3-(4-{[1-(2,3-dihydroxypropyl)piperidin-4-yl]amino}-1-(2,2,2-trifluoroethyl)-1H- |
| | indol-2-yl)prop-2-yn-1-yl]amino}-3-methoxybenzoic acid |
| 678. | methyl 4-{[3-(4-{[1-(2,3-dihydroxypropyl)piperidin-4-yl]amino}-1-(2,2,2-trifluoroethyl)- |
| | 1H-indol-2-yl)prop-2-yn-1-yl]amino}-3-methoxybenzoate |
| 679. | methyl 3-methoxy-4-[(3-{4-[(oxan-4-yl)amino]-1-(2,2,2-trifluoroethyl)-1H-indol-2- |
| | yl}prop-2-yn-1-yl)amino]benzoate |
| 680. | 3-methoxy-4-({3-[4-({1-[2-(4-methylpiperazin-1-yl)-2-oxoethyl]piperidin-4-yl}amino)-1- |
| | (2,2,2-trifluoroethyl)-1H-indol-2-yl]prop-2-yn-1-yl}amino)benzamide |
| 681. | 4-[(2-{3-[(4-methanesulfonyl-2-methoxyphenyl)amino]prop-1-yn-1-yl}-1-(2,2,2- |
| | trifluoroethyl)-1H-indol-4-yl)amino]-N-methylpiperidine-1-carboximidamide |
| 682. | 2-{3-[(6-methanesulfonyl-4-methoxypyridin-3-yl)amino]prop-1-yn-1-yl}-N-(1- |
| | methylpiperidin-4-yl)-1-(2,2,2-trifluoroethyl)-1H-indol-4-amine |
| 683. | 2-{3-[(4-methanesulfonyl-2-methoxyphenyl)amino]prop-1-yn-1-yl}-N-[1-(pyridin-4- |
| | yl)piperidin-4-yl]-1-(2,2,2-trifluoroethyl)-1H-indol-4-amine |
| 684. | 3-(4-{[2-(3-{[4-(ethanesulfonyl)-2-methoxyphenyl]amino}prop-1-yn-1-yl)-1-(2,2,2- |
| | trifluoroethyl)-1H-indol-4-yl]amino}piperidin-1-yl)propane-1,2-diol |
| 685. | 2-(3-{[4-(ethanesulfonyl)-2-methoxyphenyl]amino}prop-1-yn-1-yl)-N-(1- |
| | methylpiperidin-4-yl)-1-(2,2,2-trifluoroethyl)-1H-indol-4-amine |
| 686. | 2-hydroxy-S-{3-methoxy-4-[(3-{4-[(oxan-4-yl)amino]-1-(2,2,2-trifluoroethyl)-1H-indol- |
| | 2-yl}prop-2-yn-1-yl)amino]phenyl}ethane-1-sulfonamido |
| 687. | 2-(3-{[4-methanesulfonyl-2-(2,2,2-trifluoroethoxy)phenyl]amino}prop-1-yn-1-yl)-N-(1- |
| | methylpiperidin-4-yl)-1-(2,2,2-trifluoroethyl)-1H-indol-4-amine |
| 688. | 4-{[2-(3-{[4-methanesulfonyl-2-(2,2,2-trifluoroethoxy)phenyl]amino}prop-1-yn-1-yl)-1- |
| | (2,2,2-trifluoroethyl)-1H-indol-4-yl]amino}-1λ⁶-thiane-1,1-dione |
| 689. | 2-hydroxy-S-(3-methoxy-4-{[3-(4-{[1-(oxan-4-yl)piperidin-4-yl]amino}-1-(2,2,2- |
| | trifluoroethyl)-1H-indol-2-yl)prop-2-yn-1-yl]amino}phenyl)ethane-1-sulfonamido |
| 690. | S-(4-{[3-(4-{[1-(2,3-dihydroxypropyl)piperidin-4-yl]amino}-1-(2,2,2-trifluoroethyl)-1H- |
| | indol-2-yl)prop-2-yn-1-yl]amino}-3-methoxyphenyl)-2-hydroxyethane-1-sulfonamido |
| 691. | 2-{3-methoxy-4-[(3-{4-[(1-methylpiperidin-4-yl)amino]-1-(2,2,2-trifluoroethyl)-1H- |
| | indol-2-yl}prop-2-yn-1-yl)amino]phenyl}-2-methylpropanenitrile |
| 692. | 3-methoxy-4-{[3-(4-{[(1S,4S)-4-[(3R,4R)-3,4-dihydroxypyrrolidin-1- |
| | yl]cyclohexyl]amino}-1-(2,2,2-trifluoroethyl)-1H-indol-2-yl)prop-2-yn-1- |
| | yl]amino}benzene-1-sulfonamide |
| 693. | 3-methoxy-4-{[3-(4-{[(1R,4R)-4-[(3R,4R)-3,4-dihydroxypyrrolidin-1- |
| | yl]cyclohexyl]amino}-1-(2,2,2-trifluoroethyl)-1H-indol-2-yl)prop-2-yn-1- |
| | yl]amino}benzene-1-sulfonamide |
| 694. | 3-methoxy-4-{[3-(4-{[(1S,4S)-4-(4-hydroxypiperidin-1-yl)cyclohexyl]amino}-1-(2,2,2- |
| | trifluoroethyl)-1H-indol-2-yl)prop-2-yn-1-yl]amino}benzene-1-sulfonamide |
| 695. | 3-methoxy-4-{[3-(4-{[(1R,4R)-4-(4-hydroxypiperidin-1-yl)cyclohexyl]amino}-1-(2,2,2- |
| | trifluoroethyl)-1H-indol-2-yl)prop-2-yn-1-yl]amino}benzene-1-sulfonamide |
| 696. | 2-{4-[(3-{4-[(1,1-dioxo-1λ⁶-thian-4-yl)amino]-1-(2,2,2-trifluoroethyl)-1H-indol-2- |
| | yl}prop-2-yn-1-yl)amino]-3-methoxyphenyl}-2-methylpropanenitrile |
| 697. | 3-methoxy-4-{[3-(4-{[(1S,4S)-4-(morpholin-4-yl)cyclohexyl]amino}-1-(2,2,2- |
| | trifluoroethyl)-1H-indol-2-yl)prop-2-yn-1-yl]amino}benzene-1-sulfonamide |
| 698. | 3-methoxy-4-{[3-(4-{[(1R,4R)-4-(morpholin-4-yl)cyclohexyl]amino}-1-(2,2,2- |
| | trifluoroethyl)-1H-indol-2-yl)prop-2-yn-1-yl]amino}benzene-1-sulfonamide |
| 699. | (3S,4S)-1-[(1S,4S)-4-[(2-{3-[(4-methanesulfonyl-2-methoxyphenyl)amino]prop-1-yn-1- |
| | yl}-1-(2,2,2-trifluoroethyl)-1H-indol-4-yl)amino]cyclohexyl]pyrrolidine-3,4-diol |
| 700. | 3-methoxy-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzene-1-sulfonamide |
| 701. | 3-methoxy-4-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}benzene-1-sulfonamide |
| 702. | 2-{3-[(4-methanesulfonyl-2-methoxyphenyl)amino]prop-1-yn-1-yl}-N,N-dimethyl-1- |
| | (2,2,2-trifluoroethyl)-1H-indol-4-amine |
| 703. | 3-methoxy-4-[(3-{4-[(2-methoxyethyl)amino]-1-(2,2,2-trifluoroethyl)-1H-indol-2- |
| | yl}prop-2-yn-1-yl)amino]benzene-1-sulfonamide |
| 704. | 2-[(2-{3-[(2-methoxy-4-sulfamoylphenyl)amino]prop-1-yn-1-yl}-1-(2,2,2-trifluoroethyl)- |
| | 1H-indol-4-yl)amino]acetamide |
| 705. | 4-[(3-{4-[(1,5-dihydroxypentan-3-yl)amino]-1-(2,2,2-trifluoroethyl)-1H-indol-2-yl}prop- |
| | 2-yn-1-yl)amino]-3-methoxybenzene-1-sulfonamide |
| 706. | N-(2-{3-[(4-methanesulfonyl-2-methoxyphenyl)amino]prop-1-yn-1-yl}-1-(2,2,2- |
| | trifluoroethyl)-1H-indol-4-yl)acetamide |
| 707. | 3-(2-{3-[(4-methanesulfonyl-2-methoxyphenyl)amino]prop-1-yn-1-yl}-1-(2,2,2- |
| | trifluoroethyl)-1H-indol-4-yl)-1-(1-methylpiperidin-4-yl)urea |
| 708. | 3-methoxy-4-{[3-(4-{[(1-methylpiperidin-4-yl)carbamoyl]amino}-1-(2,2,2- |
| | trifluoroethyl)-1H-indol-2-yl)prop-2-yn-1-yl]amino}benzamide |
| 709. | N-(2-{3-[(4-methanesulfonyl-2-methoxyphenyl)amino]prop-1-yn-1-yl}-1-(2,2,2- |
| | trifluoroethyl)-1H-indol-4-yl)-4-methylpiperazine-1-carboxamide |
| 710. | N-(2-{3-[(4-carbamoyl-2-methoxyphenyl)amino]prop-1-yn-1-yl}-1-(2,2,2-trifluoroethyl)- |
| | 1H-indol-4-yl)-4-methylpiperazine-1-carboxamide |
| 711. | 3-(2-{3-[(4-methanesulfonyl-2-methoxyphenyl)amino]prop-1-yn-1-yl}-1-(2,2,2- |
| | trifluoroethyl)-1H-indol-4-yl)-1-[(1S,4S)-4-(dimethylamino)cyclohexyl]urea |
| 712. | 1-(2-(3-((2-methoxy-4-(methylsulfonyl)phenyl)amino)prop-1-yn-1-yl)-1-(2,2,2- |
| | trifluoroethyl)-1H-indol-4-yl)-3-(pyridin-4-yl)urea |
| 713. | 3-(2-{3-[(4-methanesulfonyl-2-methoxyphenyl)amino]prop-1-yn-1-yl}-1-(2,2,2- |
| | trifluoroethyl)-1H-indol-4-yl)-1-[(1R,4R)-4-(dimethylamino)cyclohexyl]urea |
| 714. | 3-(2-{3-[(4-methanesulfonyl-2-methoxyphenyl)amino]prop-1-yn-1-yl}-1-(2,2,2- |
| | trifluoroethyl)-1H-indol-4-yl)-1-[1-(2-methoxyethyl)piperidin-4-yl]urea |
| 715. | 3-(2-{3-[(4-methanesulfonyl-2-methoxyphenyl)amino]prop-1-yn-1-yl}-1-(2,2,2- |
| | trifluoroethyl)-1H-indol-4-yl)-1-[1-(oxan-4-yl)piperidin-4-yl]urea |
| 716. | 1-[1-(2,3-dihydroxypropyl)piperidin-4-yl]-3-(2-{3-[(4-methanesulfonyl-2- |
| | methoxyphenyl)amino]prop-1-yn-1-yl}-1-(2,2,2-trifluoroethyl)-1H-indol-4-yl)urea |
| 717. | 2-(4-{[(2-{3-[(4-methanesulfonyl-2-methoxyphenyl)amino]prop-1-yn-1-yl}-1-(2,2,2- |
| | trifluoroethyl)-1H-indol-4-yl)carbamoyl]amino}piperidin-1-yl)acetamide |
| 718. | 3-methoxy-4-({3-[4-({2-oxaspiro[3.3]heptan-6-yl}amino)-1-(2,2,2-trifluoroethyl)-1H- |
| | indol-2-yl]prop-2-yn-1-yl}amino)benzene-1-sulfonamide |
| 719. | [1-(chloromethyl)-3-[(2-{3-[(4-methanesulfonyl-2-methoxyphenyl)amino]prop-1-yn-1- |
| | yl}-1-(2,2,2-trifluoroethyl)-1H-indol-4-yl)amino]cyclobutyl]methanol |
| 720. | 2-{3-[(4-methanesulfonyl-2-methoxyphenyl)amino]prop-1-yn-1-yl}-N-{2- |
| | oxaspiro[3.3]heptan-6-yl}-1-(2,2,2-trifluoroethyl)-1H-indol-4-amine |
| 721. | 4-({3-[4-({2-azaspiro[3.3]heptan-6-yl}amino)-1-(2,2,2-trifluoroethyl)-1H-indol-2- |
| | yl]prop-2-yn-1-yl}amino)-3-methoxybenzene-1-sulfonamide |
| 722. | N-{2-azaspiro[3.3]heptan-6-yl}-2-{3-[(4-methanesulfonyl-2-methoxyphenyl)amino]prop- |
| | 1-yn-1-yl}-1-(2,2,2-trifluoroethyl)-1H-indol-4-amine |
| 723. | 3-methoxy-N-methyl-4-{[3-(4-{[(1R,4R)-4-(methylamino)cyclohexyl]amino}-1-(2,2,2- |
| | trifluoroethyl)-1H-indol-2-yl)prop-2-yn-1-yl]amino}benzamide |
| 724. | 3-methoxy-N-methyl-4-{[3-(4-{[(1R,4R)-4-aminocyclohexyl]amino}-1-(2,2,2- |
| | trifluoroethyl)-1H-indol-2-yl)prop-2-yn-1-yl]amino}benzamide |
| 725. | 3-methoxy-N-methyl-4-{[3-(4-{[(1S,4S)-4-aminocyclohexyl]amino}-1-(2,2,2- |
| | trifluoroethyl)-1H-indol-2-yl)prop-2-yn-1-yl]amino}benzamide |
| 726. | re1-(1R,3R)-N¹-(2-{3-[(4-methanesulfonyl-2-methoxyphenyl)amino]prop-1-yn-1-yl}-1- |
| | (2,2,2-trifluoroethyl)-1H-indol-4-yl)cyclohexane-1,3-diamine |
| 727. | rac-(1R,3S)-N¹-(2-{3-[(4-methanesulfonyl-2-methoxyphenyl)amino]prop-1-yn-1-yl}-1- |
| | (2,2,2-trifluoroethyl)-1H-indol-4-yl)cyclohexane-1,3-diamine |
| 728. | (1R,2S)-N¹-(2-{3-[(4-methanesulfonyl-2-methoxyphenyl)amino]prop-1-yn-1-yl}-1- |
| | (2,2,2-trifluoroethyl)-1H-indol-4-yl)cyclohexane-1,2-diamineQ |
| 729. | rac-(1R,2S)-N¹-(2-{3-[(4-methanesulfonyl-2-methoxyphenyl)amino]prop-1-yn-1-yl}-1- |
| | (2,2,2-trifluoroethyl)-1H-indol-4-yl)cyclohexane-1,2-diamine |
| 730. | re1-(1R,3S)-3-[(2-{3-[(4-methanesulfonyl-2-methoxyphenyl)amino]prop-1-yn-1-yl}-1- |
| | (2,2,2-trifluoroethyl)-1H-indol-4-yl)amino]cyclohexan-1-ol |
| 731. | 3-methoxy-N-methyl-4-{[3-(4-{[(1R,4R)-4-hydroxy-4-methylcyclohexyl]amino}-1- |
| | (2,2,2-trifluoroethyl)-1H-indol-2-yl)prop-2-yn-1-yl]amino}benzamide |
| 732. | 3-methoxy-N-methyl-4-{[3-(4-{[(1S,4S)-4-hydroxy-4-methylcyclohexyl]amino}-1- |
| | (2,2,2-trifluoroethyl)-1H-indol-2-yl)prop-2-yn-1-yl]amino}benzamide |
| 733. | 4-[(3-{4-[(4-cyanocyclohexyl)amino]-1-(2,2,2-trifluoroethyl)-1H-indol-2-yl}prop-2-yn-1- |
| | yl)amino]-3-methoxy-N-methylbenzamide |
| 734. | 3-methoxy-N-methyl-4-{[3-(4-{[(1R,4R)-4-cyanocyclohexyl]amino}-1-(2,2,2- |
| | trifluoroethyl)-1H-indol-2-yl)prop-2-yn-1-yl]amino}benzamide |
| 735. | 3-methoxy-N-methyl-4-{[3-(4-{[(1S,4S)-4-cyanocyclohexyl]amino}-1-(2,2,2- |
| | trifluoroethyl)-1H-indol-2-yl)prop-2-yn-1-yl]amino}benzamide |
| 736. | 3-[(2-{3-[(4-methanesulfonyl-2-methoxyphenyl)amino]prop-1-yn-1-yl}-1-(2,2,2- |
| | trifluoroethyl)-1H-indol-4-yl)amino]cyclohexane-1-carboxylic acid |
| 737. | 2-fluoro-N¹-(2-(3-((2-methoxy-4-(methylsulfonyl)phenyl)amino)prop-1-yn-1-yl)-1-(2,2,2- |
| | trifluoroethyl)-1H-indol-4-yl)cyclohexane-1,4-diamine |
| 738. | (1R,2R,4S)-2-fluoro-N¹-(2-(3-((2-methoxy-4-(methylsulfonyl)phenyl)amino)prop-1-yn-1- |
| | yl)-1-(2,2,2-trifluoroethyl)-1H-indol-4-yl)cyclohexane-1,4-diamine |
| 739. | 2-fluoro-N¹-(2-{3-[(4-methanesulfonyl-2-methoxyphenyl)amino]prop-1-yn-1-yl}-1- |
| | (2,2,2-trifluoroethyl)-1H-indol-4-yl)-N⁴-methylcyclohexane-1,4-diamine |
| 740. | (1R,2R,4S)-2-fluoro-N¹-(2-(3-((2-methoxy-4-(methylsulfonyl)phenyl)amino)prop-1-yn-1- |
| | yl)-1-(2,2,2-trifluoroethyl)-1H-indol-4-yl)-N⁴-methylcyclohexane-1,4-diamine |
| 741. | 2-fluoro-N¹-(2-(3-((2-methoxy-4-(methylsulfonyl)phenyl)amino)prop-1-yn-1-yl)-1-(2,2,2- |
| | trifluoroethyl)-1H-indol-4-yl)-N⁴,N⁴-dimethylcyclohexane-1,4-diamine |
| 742. | (1R,2R,4S)-2-fluoro-N¹-(2-(3-((2-methoxy-4-(methylsulfonyl)phenyl)amino)prop-1-yn-1- |
| | yl)-1-(2,2,2-trifluoroethyl)-1H-indol-4-yl)-N⁴,N⁴-dimethylcyclohexane-1,4-diamine |
| 743. | (3S,4R)-3-fluoro-4-[(2-{3-[(4-methanesulfonyl-2-methoxyphenyl)amino]prop-1-yn-1-yl}- |
| | 1-(2,2,2-trifluoroethyl)-1H-indol-4-yl)amino]cyclohexan-1-ol |
| 744. | 3-methoxy-4-{[3-(4-{[(1R,4R)-4-[bis(2-hydroxyethyl)amino]cyclohexyl]amino}-1- |
| | (2,2,2-trifluoroethyl)-1H-indol-2-yl)prop-2-yn-1-yl]amino}benzene-1-sulfonamide |
| 745. | 3-methoxy-4-{[3-(4-{[(1S,4S)-4-[bis(2-hydroxyethyl)amino]cyclohexyl]amino}-1-(2,2,2- |
| | trifluoroethyl)-1H-indol-2-yl)prop-2-yn-1-yl]amino}benzene-1-sulfonamide |
| 746. | 4-((3-(4-(((1R,4R)-4-(dimethylamino)cyclohexyl)amino)-1-(2,2,2-trifluoroethyl)-1H- |
| | indol-2-yl)prop-2-yn-1-yl)amino)-3-methoxybenzenesulfonamide |
| 747. | 4-((3-(4-(((1S,4S)-4-(dimethylamino)cyclohexyl)amino)-1-(2,2,2-trifluoroethyl)-1H- |
| | indol-2-yl)prop-2-yn-1-yl)amino)-3-methoxybenzenesulfonamide |
| 748. | 3-methoxy-N-(1,2-oxazol-3-yl)-4-{[3-(4-{[(1R,4R)-4- |
| | (dimethylamino)cyclohexyl]amino}-1-(2,2,2-trifluoroethyl)-1H-indol-2-yl)prop-2-yn-1- |
| | yl]amino}benzene-1-sulfonamide |
| 749. | 3-methoxy-N-(oxan-4-yl)-4-{[3-(4-{[(1R,4R)-4-(dimethylamino)cyclohexyl]amino}-1- |
| | (2,2,2-trifluoroethyl)-1H-indol-2-yl)prop-2-yn-1-yl]amino}benzene-1-sulfonamide |
| 750. | 3-methoxy-N-(1,2-oxazol-3-yl)-4-{[3-(4-{[(1S,4S)-4- |
| | (dimethylamino)cyclohexyl]amino}-1-(2,2,2-trifluoroethyl)-1H-indol-2-yl)prop-2-yn-1- |
| | yl]amino}benzene-1-sulfonamide |
| 751. | 3-methoxy-N-(oxan-4-yl)-4-{[3-(4-{[(1S,4S)-4-(dimethylamino)cyclohexyl]amino}-1- |
| | (2,2,2-trifluoroethyl)-1H-indol-2-yl)prop-2-yn-1-yl]amino}benzene-1-sulfonamide |
| 752. | N-(3-(4-(((1S,4S)-4-(dimethylamino)cyclohexyl)amino)-1-(2,2,2-trifluoroethyl)-1H- |
| | indol-2-yl)prop-2-yn-1-yl)-N-(2-hydroxy-4-(methylsulfonyl)phenyl)propionamide |
| 753. | N-(3-(4-(((1R,4R)-4-(dimethylamino)cyclohexyl)amino)-1-(2,2,2-trifluoroethyl)-1H- |
| | indol-2-yl)prop-2-yn-1-yl)-N-(2-hydroxy-4-(methylsulfonyl)phenyl)propionamide |
| 754. | 3-methoxy-N-(5-methyl-1,2-oxazol-3-yl)-4-{[3-(4-{[(1S,4S)-4- |
| | (dimethylamino)cyclohexyl]amino}-1-(2,2,2-trifluoroethyl)-1H-indol-2-yl)prop-2-yn-1- |
| | yl]amino}benzene-1-sulfonamide |
| 755. | 3-methoxy-N-(5-methyl-1,2-oxazol-3-yl)-4-{[3-(4-{[(1R,4R)-4- |
| | (dimethylamino)cyclohexyl]amino}-1-(2,2,2-trifluoroethyl)-1H-indol-2-yl)prop-2-yn-1- |
| | yl]amino}benzene-1-sulfonamide |
| 756. | (1S,4S)-N⁴-(2-{3-[(4-methanesulfonyl-2-methoxyphenyl)amino]prop-1-yn-1-yl}-1- |
| | (2,2,2-trifluoroethyl)-1H-indol-4-yl)-N¹-(2-methoxyethyl)-N¹-methylcyclohexane-1,4- |
| | diamine |
| 757. | (1R,4R)-N⁴-(2-{3-[(4-methanesulfonyl-2-methoxyphenyl)amino]prop-1-yn-1-yl}-1- |
| | (2,2,2-trifluoroethyl)-1H-indol-4-yl)-N¹-(2-methoxyethyl)-N¹-methylcyclohexane-1,4- |
| | diamine |
| 758. | 3-methoxy-4-{[3-(4-{[(1S,4S)-4-[(3S,4S)-3,4-dihydroxypyrrolidin-1- |
| | yl]cyclohexyl]amino}-1-(2,2,2-trifluoroethyl)-1H-indol-2-yl)prop-2-yn-1- |
| | yl]amino}benzene-1-sulfonamide |
| 759. | 4-((3-(4-(((1R,4R)-4-((3R,4R)-3,4-dihydroxypyrrolidin-1-yl)cyclohexyl)amino)-1-(2,2,2- |
| | trifluoroethyl)-1H-indol-2-yl)prop-2-yn-1-yl)amino)-3-methoxybenzenesulfonamide |
| 760. | 4-((3-(4-(((1R,4S)-4-((3S,4S)-3,4-dihydroxypyrrolidin-1-yl)cyclohexyl)amino)-1-(2,2,2- |
| | trifluoroethyl)-1H-indol-2-yl)prop-2-yn-1-yl)amino)-3-methoxybenzenesulfonamide |
| 761. | 3-methoxy-4-{[3-(4-{[(1R,4R)-4-[bis(2-methoxyethyl)amino]cyclohexyl]amino}-1- |
| | (2,2,2-trifluoroethyl)-1H-indol-2-yl)prop-2-yn-1-yl]amino}benzene-1-sulfonamide |
| 762. | 3-methoxy-4-{[3-(4-{[(1S,4S)-4-[bis(2-methoxyethyl)amino]cyclohexyl]amino}-1-(2,2,2- |
| | trifluoroethyl)-1H-indol-2-yl)prop-2-yn-1-yl]amino}benzene-1-sulfonamide |
| 763. | 4-({3-[4-({1,4-dioxaspiro[4.5]decan-8-yl}amino)-1-(2,2,2-trifluoroethyl)-1H-indol-2- |
| | yl]prop-2-yn-1-yl}amino)-3-methoxybenzene-1-sulfonamide |
| 764. | N-{1,4-dioxaspiro[4.5]decan-8-yl}-2-{3-[(4-methanesulfonyl-2- |
| | methoxyphenyl)amino]prop-1-yn-1-yl}-1-(2,2,2-trifluoroethyl)-1H-indol-4-amine |
| 765. | 3-methoxy-4-[(3-{4-[(4-oxocyclohexyl)amino]-1-(2,2,2-trifluoroethyl)-1H-indol-2- |
| | yl}prop-2-yn-1-yl)amino]benzene-1-sulfonamide |
| 766. | 4-[(2-{3-[(4-methanesulfonyl-2-methoxyphenyl)amino]prop-1-yn-1-yl}-1-(2,2,2- |
| | trifluoroethyl)-1H-indol-4-yl)amino]cyclohexan-1-one |
| 767. | (1R,4R)-N⁴-[2-(3-{[2-methoxy-4-(morpholine-4-sulfonyl)phenyl]amino}prop-1-yn-1- |
| | yl)-1-(2,2,2-trifluoroethyl)-1H-indol-4-yl]-N¹,N¹-dimethylcyclohexane-1,4-diamine |
| 768. | (1S,4S)-N⁴-[2-(3-{[2-methoxy-4-(morpholine-4-sulfonyl)phenyl]amino}prop-1-yn-1-yl)- |
| | 1-(2,2,2-trifluoroethyl)-1H-indol-4-yl]-N¹,N¹-dimethylcyclohexane-1,4-diamine |
| 769. | N,N-bis(2-hydroxyethyl)-3-methoxy-4-{[3-(4-{[(1S,4S)-4- |
| | (dimethylamino)cyclohexyl]amino}-1-(2,2,2-trifluoroethyl)-1H-indol-2-yl)prop-2-yn-1- |
| | yl]amino}benzene-1-sulfonamide |
| 770. | N,N-bis(2-hydroxyethyl)-3-methoxy-4-{[3-(4-{[(1R,4R)-4- |
| | (dimethylamino)cyclohexyl]amino}-1-(2,2,2-trifluoroethyl)-1H-indol-2-yl)prop-2-yn-1- |
| | yl]amino}benzene-1-sulfonamide |
| 771. | 3-methoxy-N-methyl-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzene-1-sulfonamide |
| 772. | 3-methoxy-N-methyl-4-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}benzene-1-sulfonamide |
| 773. | 3-methoxy-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzamide |
| 774. | 3-methoxy-N-methyl-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzamide |
| 775. | 3-methoxy-N-methyl-4-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}benzamide |
| 776. | (1R,4R)-N⁴-{2-[3-({2-methoxy-4-[(4-methylpiperazin-1-yl)sulfonyl]phenyl}amino)prop- |
| | 1-yn-1-yl]-1-(2,2,2-trifluoroethyl)-1H-indol-4-yl}-N¹,N¹-dimethylcyclohexane-1,4- |
| | diamine |
| 777. | (1R,4R)-N⁴-(2-{3-[(4-methanesulfonyl-2-methoxyphenyl)amino]prop-1-yn-1-yl}-1- |
| | [(oxiran-2-yl)methyl]-1H-indol-4-yl)-N¹,N¹-dimethylcyclohexane-1,4-diamine |
| 778. | 2-[(2-{3-[(4-methanesulfonyl-2-methoxyphenyl)amino]prop-1-yn-1-yl}-4-{[(1R,4R)-4- |
| | (dimethylamino)cyclohexyl]amino}-1H-indol-1-yl)methyl]prop-2-enenitrile |
| 779. | 5-methanesulfonyl-2-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}phenol |
| 780. | 5-methanesulfonyl-2-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}phenol |
| 781. | 2-(5-methanesulfonyl-2-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}phenoxy)acetonitrile |
| 782. | 2-(5-methanesulfonyl-2-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}phenoxy)acetonitrile |
| 783. | N-(3-methoxy-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzenesulfonyl)acetamide |
| 784. | N-(3-methoxy-4-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}benzenesulfonyl)acetamide |
| 785. | N-(3-methoxy-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzenesulfonyl)propanamide |
| 786. | N-(3-methoxy-4-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}benzenesulfonyl)propanamide |
| 787. | 2-(5-methanesulfonyl-2-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}phenoxy)ethan-1-ol |
| 788. | 2-(5-methanesulfonyl-2-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}phenoxy)ethan-1-ol |
| 789. | (1R,4R)-N⁴-[2-(3-{[4-methanesulfonyl-2-(2-methoxyethoxy)phenyl]amino}prop-1-yn-1- |
| | yl)-1-(2,2,2-trifluoroethyl)-1H-indol-4-yl]-N¹,N¹-dimethylcyclohexane-1,4-diamine |
| 790. | N-(3-(4-(((1R,4R)-4-(dimethylamino)cyclohexyl)amino)-1-(2,2,2-trifluoroethyl)-1H- |
| | indol-2-yl)prop-2-yn-1-yl)-N-(2-hydroxy-4-(methylsulfonyl)phenyl)isobutyramide |
| 791. | N-(3-(4-(((1S,4S)-4-(dimethylamino)cyclohexyl)amino)-1-(2,2,2-trifluoroethyl)-1H- |
| | indol-2-yl)prop-2-yn-1-yl)-N-(2-hydroxy-4-(methylsulfonyl)phenyl)isobutyramide |
| 792. | (1R,4R)-N⁴-[2-(3-{[4-methanesulfonyl-2-(2,2,2-trifluoroethoxy)phenyl]amino}prop-1- |
| | yn-1-yl)-1-(2,2,2-trifluoroethyl)-1H-indol-4-yl]-N¹,N¹-dimethylcyclohexane-1,4-diamine |
| 793. | (1S,4S)-N⁴-[2-(3-{[4-methanesulfonyl-2-(2,2,2-trifluoroethoxy)phenyl]amino}prop-1- |
| | yn-1-yl)-1-(2,2,2-trifluoroethyl)-1H-indol-4-yl]-N¹,N¹-dimethylcyclohexane-1,4-diamine |
| 794. | (1S,4S)-N⁴-{2-[3-(2-amino-4-methanesulfonylphenoxy)prop-1-yn-1-yl]-1-(2,2,2- |
| | trifluoroethyl)-1H-indol-4-yl}-N¹,N¹-dimethylcyclohexane-1,4-diamine |
| 795. | (1R,4R)-N⁴-(2-{3-[(2-ethoxy-4-methanesulfonylphenyl)amino]prop-1-yn-1-yl}-1-(2,2,2- |
| | trifluoroethyl)-1H-indol-4-yl)-N¹,N¹-dimethylcyclohexane-1,4-diamine |
| 796. | (1S,4S)-N⁴-(2-{3-[(2-ethoxy-4-methanesulfonylphenyl)amino]prop-1-yn-1-yl}-1-(2,2,2- |
| | trifluoroethyl)-1H-indol-4-yl)-N¹,N¹-dimethylcyclohexane-1,4-diamine |
| 797. | 3-hydroxy-N-methyl-4-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}benzamide |
| 798. | 3-hydroxy-N-methyl-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzamide |
| 799. | 3-(2-methoxyethoxy)-N-methyl-4-{[3-(4-{[(1S,4S)-4- |
| | (dimethylamino)cyclohexyl]amino}-1-(2,2,2-trifluoroethyl)-1H-indol-2-yl)prop-2-yn-1- |
| | yl]amino}benzamide |
| 800. | (1R,4R)-N⁴-{2-[3-(2-amino-4-methanesulfonylphenoxy)prop-1-yn-1-yl]-1-(2,2,2- |
| | trifluoroethyl)-1H-indol-4-yl}-N¹,N¹-dimethylcyclohexane-1,4-diamine |
| 801. | N-ethyl-3-methoxy-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzamide |
| 802. | N-ethyl-3-methoxy-4-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}benzamide |
| 803. | 3-(2-methoxyethoxy)-N-methyl-4-{[3-(4-{[(1R,4R)-4- |
| | (dimethylamino)cyclohexyl]amino}-1-(2,2,2-trifluoroethyl)-1H-indol-2-yl)prop-2-yn-1- |
| | yl]amino}benzamide |
| 804. | 3-(2-fluoroethoxy)-N-methyl-4-{[3-(4-{[(1R,4R)-4-(dimethylamino)cyclohexyl]amino}- |
| | 1-(2,2,2-trifluoroethyl)-1H-indol-2-yl)prop-2-yn-1-yl]amino}benzamide |
| 805. | 3-(2-fluoroethoxy)-N-methyl-4-{[3-(4-{[(1S,4S)-4-(dimethylamino)cyclohexyl]amino}- |
| | 1-(2,2,2-trifluoroethyl)-1H-indol-2-yl)prop-2-yn-1-yl]amino}benzamide |
| 806. | 3-(fluoromethoxy)-N-methyl-4-{[3-(4-{[(1R,4R)-4-(dimethylamino)cyclohexyl]amino}- |
| | 1-(2,2,2-trifluoroethyl)-1H-indol-2-yl)prop-2-yn-1-yl]amino}benzamide |
| 807. | 3-(fluoromethoxy)-N-methyl-4-{[3-(4-{[(1S,4S)-4-(dimethylamino)cyclohexyl]amino}-1- |
| | (2,2,2-trifluoroethyl)-1H-indol-2-yl)prop-2-yn-1-yl]amino}benzamide |
| 808. | (1S,4S)-N⁴-(2-{3-[4-methanesulfonyl-2-(methylamino)phenoxy]prop-1-yn-1-yl}-1- |
| | (2,2,2-trifluoroethyl)-1H-indol-4-yl)-N¹,N¹-dimethylcyclohexane-1,4-diamine |
| 809. | (1R,4R)-N⁴-(2-{3-[4-methanesulfonyl-2-(methylamino)phenoxy]prop-1-yn-1-yl}-1- |
| | (2,2,2-trifluoroethyl)-1H-indol-4-yl)-N¹,N¹-dimethylcyclohexane-1,4-diamine |
| 810. | 3-methoxy-N-methyl-4-{[3-(4-{[(1S,4S)-4-(methylamino)cyclohexyl]amino}-1-(2,2,2- |
| | trifluoroethyl)-1H-indol-2-yl)prop-2-yn-1-yl]amino}benzamide |
| 811. | (1S,4S)-N⁴-(2-{3-[2-(dimethylamino)-4-methanesulfonylphenoxy]prop-1-yn-1-yl}-1- |
| | (2,2,2-trifluoroethyl)-1H-indol-4-yl)-N¹,N¹-dimethylcyclohexane-1,4-diamine |
| 812. | (1R,4R)-N⁴-(2-{3-[2-(dimethylamino)-4-methanesulfonylphenoxy]prop-1-yn-1-yl}-1- |
| | (2,2,2-trifluoroethyl)-1H-indol-4-yl)-N¹,N¹-dimethylcyclohexane-1,4-diamine |
| 813. | 3-(cyanomethoxy)-N-methyl-4-{[3-(4-{[(1R,4R)-4-(dimethylamino)cyclohexyl]amino}- |
| | 1-(2,2,2-trifluoroethyl)-1H-indol-2-yl)prop-2-yn-1-yl]amino}benzamide |
| 814. | 3-(2-cyanoethoxy)-N-methyl-4-{[3-(4-{[(1R,4R)-4-(dimethylamino)cyclohexyl]amino}- |
| | 1-(2,2,2-trifluoroethyl)-1H-indol-2-yl)prop-2-yn-1-yl]amino}benzamide |
| 815. | N-(5-methanesulfonyl-2-{[3-(4-{[(1R,4R)-4-(dimethylamino)cyclohexyl]amino}-1- |
| | (2,2,2-trifluoroethyl)-1H-indol-2-yl)prop-2-yn-1-yl]oxy}phenyl)acetamide |
| 816. | N-(2-((3-(4-(((1S,4S)-4-(dimethylamino)cyclohexyl)amino)-1-(2,2,2-trifluoroethyl)-1H- |
| | indol-2-yl)prop-2-yn-1-yl)oxy)-5-(methylsulfonyl)phenyl)acetamide |
| 817. | (1R,4R)-N¹-(2-(3-((2-(fluoromethoxy)-4-(methylsulfonyl)phenyl)amino)prop-1-yn-1-yl)- |
| | 1-(2,2,2-trifluoroethyl)-1H-indol-4-yl)-N⁴,N⁴-dimethylcyclohexane-1,4-diamine |
| 818. | (1S,4S)-N¹-(2-(3-((2-(fluoromethoxy)-4-(methylsulfonyl)phenyl)amino)prop-1-yn-1-yl)- |
| | 1-(2,2,2-trifluoroethyl)-1H-indol-4-yl)-N⁴,N⁴-dimethylcyclohexane-1,4-diamine |
| 819. | (1R,4R)-N⁴-[2-(3-{[4-(ethanesulfonyl)-2-methoxyphenyl]amino}prop-1-yn-1-yl)-1- |
| | (2,2,2-trifluoroethyl)-1H-indol-4-yl]-N¹,N¹-dimethylcyclohexane-1,4-diamine |
| 820. | (1S,4S)-N⁴-[2-(3-{[4-(ethanesulfonyl)-2-methoxyphenyl]amino}prop-1-yn-1-yl)-1-(2,2,2- |
| | trifluoroethyl)-1H-indol-4-yl]-N¹,N¹-dimethylcyclohexane-1,4-diamine |
| 821. | 2-(3-methoxy-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}phenyl)-2-methylpropanenitrile |
| 822. | 2-(4-((3-(4-(((1S,4S)-4-(dimethylamino)cyclohexyl)amino)-1-(2,2,2-trifluoroethyl)-1H- |
| | indol-2-yl)prop-2-yn-1-yl)amino)-3-methoxyphenyl)-2-methylpropanenitrile |
| 823. | 3-(cyanomethoxy)-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzene-1-sulfonamide |
| 824. | 3-(cyanomethoxy)-4-((3-(4-(((1S,4S)-4-(dimethylamino)cyclohexyl)amino)-1-(2,2,2- |
| | trifluoroethyl)-1H-indol-2-yl)prop-2-yn-1-yl)amino)benzenesulfonamide |
| 825. | 3-(3-(4-(((1R,4R)-4-(dimethylamino)cyclohexyl)amino)-1-(2,2,2-trifluoroethyl)-1H- |
| | indol-2-yl)prop-2-yn-1-yl)-6-(methylsulfonyl)benzo[d]oxazol-2(3H)-one |
| 826. | 3-(3-(4-(((1S,4S)-4-(dimethylamino)cyclohexyl)amino)-1-(2,2,2-trifluoroethyl)-1H-indol- |
| | 2-yl)prop-2-yn-1-yl)-6-(methylsulfonyl)benzo[d]oxazol-2(3H)-one |
| 827. | 3-methoxy-N,N-dimethyl-4-{[3-(4-{[(1R,4R)-4-(dimethylamino)cyclohexyl]amino}-1- |
| | (2,2,2-trifluoroethyl)-1H-indol-2-yl)prop-2-yn-1-yl]amino}benzene-1-sulfonamide |
| 828. | (1R,4R)-N¹-(2-(3-(((3S,4R)-3-methoxytetrahydro-2H-pyran-4-yl)amino)prop-1-yn-1-yl)- |
| | 1-(2,2,2-trifluoroethyl)-1H-indol-4-yl)-N⁴,N⁴-dimethylcyclohexane-1,4-diamine |
| 829. | (1S,4S)-N¹-(2-(3-(((3S,4R)-3-methoxytetrahydro-2H-pyran-4-yl)amino)prop-1-yn-1-yl)- |
| | 1-(2,2,2-trifluoroethyl)-1H-indol-4-yl)-N⁴,N⁴-dimethylcyclohexane-1,4-diamine |
| 830. | (1R,4R)-N⁴-(2-{3-[(4-chloro-2-methoxyphenyl)amino]prop-1-yn-1-yl}-1-(2,2,2- |
| | trifluoroethyl)-1H-indol-4-yl)-N¹,N¹-dimethylcyclohexane-1,4-diamine |
| 831. | (1S,4S)-N¹-(2-(3-((4-chloro-2-methoxyphenyl)amino)prop-1-yn-1-yl)-1-(2,2,2- |
| | trifluoroethyl)-1H-indol-4-yl)-N⁴,N⁴-dimethylcyclohexane-1,4-diamine |
| 832. | 4-((3-(4-(((1R,4R)-4-(dimethylamino)cyclohexyl)amino)-1-(2,2,2-trifluoroethyl)-1H- |
| | indol-2-yl)prop-2-yn-1-yl)amino)-2-fluoro-5-methoxybenzamide |
| 833. | 4-((3-(4-(((1S,4S)-4-(dimethylamino)cyclohexyl)amino)-1-(2,2,2-trifluoroethyl)-1H- |
| | indol-2-yl)prop-2-yn-1-yl)amino)-2-fluoro-5-methoxybenzamide |
| 834. | (1R,4R)-N⁴-[2-(3-{[2-methoxy-4-(trifluoromethyl)phenyl]amino}prop-1-yn-1-yl)-1- |
| | (2,2,2-trifluoroethyl)-1H-indol-4-yl]-N¹,N¹-dimethylcyclohexane-1,4-diamine |
| 835. | (1S,4S)-N¹-(2-(3-((2-methoxy-4-(trifluoromethyl)phenyl)amino)prop-1-yn-1-yl)-1-(2,2,2- |
| | trifluoroethyl)-1H-indol-4-yl)-N⁴,N⁴-dimethylcyclohexane-1,4-diamine |
| 836. | 2-fluoro-5-methoxy-N-methyl-4-{[3-(4-{[(1R,4R)-4-(dimethylamino)cyclohexyl]amino}- |
| | 1-(2,2,2-trifluoroethyl)-1H-indol-2-yl)prop-2-yn-1-yl]amino}benzamide |
| 837. | 4-((3-(4-(((1S,4S)-4-(dimethylamino)cyclohexyl)amino)-1-(2,2,2-trifluoroethyl)-1H- |
| | indol-2-yl)prop-2-yn-1-yl)amino)-2-fluoro-5-methoxy-N-methylbenzamide |
| 838. | (1S,4S)-N⁴-[2-(3-{[4-methanesulfonyl-2-(trifluoromethyl)phenyl]amino}prop-1-yn-1-yl)- |
| | 1-(2,2,2-trifluoroethyl)-1H-indol-4-yl]-N¹,N¹-dimethylcyclohexane-1,4-diamine |
| 839. | (1R,4R)-N⁴-[2-(3-{[4-methanesulfonyl-2-(trifluoromethyl)phenyl]amino}prop-1-yn-1-yl)- |
| | 1-(2,2,2-trifluoroethyl)-1H-indol-4-yl]-N¹,N¹-dimethylcyclohexane-1,4-diamine |
| 840. | (1R,4R)-N⁴-(2-{3-[(4-methanesulfonyl-2-methylphenyl)amino]prop-1-yn-1-yl}-1-(2,2,2- |
| | trifluoroethyl)-1H-indol-4-yl)-N¹,N¹-dimethylcyclohexane-1,4-diamine |
| 841. | (1S,4S)-N¹,N¹-dimethyl-N⁴-(2-(3-((2-methyl-4-(methylsulfonyl)phenyl)amino)prop-1-yn- |
| | 1-yl)-1-(2,2,2-trifluoroethyl)-1H-indol-4-yl)cyclohexane-1,4-diamine |
| 842. | (1R,4R)-N⁴-[2-(3-{[4-methanesulfonyl-2-(trifluoromethoxy)phenyl]amino}prop-1-yn-1- |
| | yl)-1-(2,2,2-trifluoroethyl)-1H-indol-4-yl]-N¹,N¹-dimethylcyclohexane-1,4-diamine |
| 843. | (1S,4S)-N¹,N¹-dimethyl-N⁴-(2-(3-((4-(methylsulfonyl)-2- |
| | (trifluoromethoxy)phenyl)amino)prop-1-yn-1-yl)-1-(2,2,2-trifluoroethyl)-1H-indol-4- |
| | yl)cyclohexane-1,4-diamine |
| 844. | 3-methoxy-N-methyl-4-{[3-(4-{[(1R,4R)-4-(diethylamino)cyclohexyl]amino}-1-(2,2,2- |
| | trifluoroethyl)-1H-indol-2-yl)prop-2-yn-1-yl]amino}benzamide |
| 845. | 4-((3-(4-(((1S,4S)-4-(diethylamino)cyclohexyl)amino)-1-(2,2,2-trifluoroethyl)-1H-indol- |
| | 2-yl)prop-2-yn-1-yl)amino)-3-methoxy-N-methylbenzamide |
| 846. | 3-methoxy-4-{[3-(4-{[(1R,4R)-4-(diethylamino)cyclohexyl]amino}-1-(2,2,2- |
| | trifluoroethyl)-1H-indol-2-yl)prop-2-yn-1-yl]amino}benzene-1-sulfonamide |
| 847. | 4-((3-(4-(((1S,4S)-4-(diethylamino)cyclohexyl)amino)-1-(2,2,2-trifluoroethyl)-1H-indol- |
| | 2-yl)prop-2-yn-1-yl)amino)-3-methoxybenzenesulfonamide |
| 848. | 3-methoxy-N-methyl-4-{[3-(4-{[(1R,4R)-4-(diethylamino)cyclohexyl]amino}-1-(2,2,2- |
| | trifluoroethyl)-1H-indol-2-yl)prop-2-yn-1-yl]amino}benzene-1-sulfonamide |
| 849. | 2-(5-methanesulfonyl-2-{[3-(4-{[(1R,4R)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}phenoxy)acetonitrile |
| 850. | 2-(5-methanesulfonyl-2-{[3-(4-{[(1S,4S)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}phenoxy)acetonitrile |
| 851. | 3-methoxy-N-methyl-4-{[3-(4-{[(1R,4R)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzamide |
| 852. | 3-methoxy-N-methyl-4-{[3-(4-{[(1S,4S)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzamide |
| 853. | N-ethyl-3-methoxy-4-{[3-(4-{[(1R,4R)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzamide |
| 854. | N-ethyl-3-methoxy-4-((3-(4-(((1S,4S)-4-((2- |
| | methoxyethyl)(methyl)amino)cyclohexyl)amino)-1-(2,2,2-trifluoroethyl)-1H-indol-2- |
| | yl)prop-2-yn-1-yl)amino)benzamide |
| 855. | (1R,4R)-N¹-(2-(3-((2-(fluoromethoxy)-4-(methylsulfonyl)phenyl)amino)prop-1-yn-1-yl)- |
| | 1-(2,2,2-trifluoroethyl)-1H-indol-4-yl)-N⁴-(2-methoxyethyl)-N⁴-methylcyclohexane-1,4- |
| | diamine |
| 856. | (1S,4S)-N¹-(2-(3-((2-(fluoromethoxy)-4-(methylsulfonyl)phenyl)amino)prop-1-yn-1-yl)- |
| | 1-(2,2,2-trifluoroethyl)-1H-indol-4-yl)-N⁴-(2-methoxyethyl)-N⁴-methylcyclohexane-1,4- |
| | diamine |
| 857. | 3-(fluoromethoxy)-N-methyl-4-{[3-(4-{[(1R,4R)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzamide |
| 858. | 3-(fluoromethoxy)-N-methyl-4-{[3-(4-{[(1S,4S)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzamide |
| 859. | 3-(fluoromethoxy)-4-((3-(4-(((1S,4S)-4-((2- |
| | methoxyethyl)(methyl)amino)cyclohexyl)amino)-1-(2,2,2-trifluoroethyl)-1H-indol-2- |
| | yl)prop-2-yn-1-yl)amino)-N-methylbenzamide |
| 860. | 3-methoxy-4-{[3-(4-{[(1R,4R)-4-[(2-methoxyethyl)(methyl)amino]cyclohexyl]amino}-1- |
| | (2,2,2-trifluoroethyl)-1H-indol-2-yl)prop-2-yn-1-yl]amino}benzene-1-sulfonamide |
| 861. | 3-methoxy-4-((3-(4-(((1S,4S)-4-((2-methoxyethyl)(methyl)amino)cyclohexyl)amino)-1- |
| | (2,2,2-trifluoroethyl)-1H-indol-2-yl)prop-2-yn-1-yl)amino)benzenesulfonamide |
| 862. | (1R,4R)-N¹,N¹-diethyl-N⁴-(2-{3-[(4-methanesulfonyl-2-methoxyphenyl)amino]prop-1- |
| | yn-1-yl}-1-(2,2,2-trifluoroethyl)-1H-indol-4-yl)cyclohexane-1,4-diamine |
| 863. | (1S,4S)-N¹,N¹-diethyl-N⁴-(2-(3-((2-methoxy-4-(methylsulfonyl)phenyl)amino)prop-1-yn- |
| | 1-yl)-1-(2,2,2-trifluoroethyl)-1H-indol-4-yl)cyclohexane-1,4-diamine |
| 864. | 3-(cyanomethoxy)-4-{[3-(4-{[(1R,4R)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzene-1-sulfonamide |
| 865. | 3-(cyanomethoxy)-4-{[3-(4-{[(1S,4S)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzene-1-sulfonamide |
| 866. | 3-(fluoromethoxy)-4-{[3-(4-{[(1R,4R)-4-[bis(2-methoxyethyl)amino]cyclohexyl]amino}- |
| | 1-(2,2,2-trifluoroethyl)-1H-indol-2-yl)prop-2-yn-1-yl]amino}benzene-1-sulfonamide |
| 867. | 3-(fluoromethoxy)-4-{[3-(4-{[(1S,4S)-4-[bis(2-methoxyethyl)amino]cyclohexyl]amino}- |
| | 1-(2,2,2-trifluoroethyl)-1H-indol-2-yl)prop-2-yn-1-yl]amino}benzene-1-sulfonamide |
| 868. | 3-(fluoromethoxy)-4-{[3-(4-{[(1R,4R)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzene-1-sulfonamide |
| 869. | 3-(fluoromethoxy)-4-{[3-(4-{[(1S,4S)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzene-1-sulfonamide |
| 870. | 3-methoxy-4-{[3-(4-{[(1R,4R)-4-(morpholin-4-yl)cyclohexyl]amino}-1-(2,2,2- |
| | trifluoroethyl)-1H-indol-2-yl)prop-2-yn-1-yl]amino}benzamide |
| 871. | 3-methoxy-4-{[3-(4-{[(1S,4S)-4-(morpholin-4-yl)cyclohexyl]amino}-1-(2,2,2- |
| | trifluoroethyl)-1H-indol-2-yl)prop-2-yn-1-yl]amino}benzamide |
| 872. | 3-methoxy-N-methyl-4-{[3-(4-{[(1R,4R)-4-(morpholin-4-yl)cyclohexyl]amino}-1-(2,2,2- |
| | trifluoroethyl)-1H-indol-2-yl)prop-2-yn-1-yl]amino}benzamide |
| 873. | 3-methoxy-N-methyl-4-{[3-(4-{[(1S,4S)-4-(morpholin-4-yl)cyclohexyl]amino}-1-(2,2,2- |
| | trifluoroethyl)-1H-indol-2-yl)prop-2-yn-1-yl]amino}benzamide |
| 874. | 2-(2-{3-[(4-methanesulfonyl-2-methoxyphenyl)amino]prop-1-yn-1-yl}-4-{[(1R,4R)-4- |
| | (morpholin-4-yl)cyclohexyl]amino}-1H-indol-1-yl)acetonitrile |
| 875. | 4-({3-[1-(2-fluoroethyl)-4-{[(1R,4R)-4-(morpholin-4-yl)cyclohexyl]amino}-1H-indol-2- |
| | yl]prop-2-yn-1-yl}amino)-3-methoxy-N-methylbenzamide |
| 876. | 1-(2-fluoroethyl)-2-{3-[(4-methanesulfonyl-2-methoxyphenyl)amino]prop-1-yn-1-yl}-N- |
| | [(1R,4R)-4-(morpholin-4-yl)cyclohexyl]-1H-indol-4-amine |
| 877. | 4-({3-[1-(cyanomethyl)-4-{[(1R,4R)-4-(morpholin-4-yl)cyclohexyl]amino}-1H-indol-2- |
| | yl]prop-2-yn-1-yl}amino)-3-methoxy-N-methylbenzamide |
| 878. | 2-{3-[(4-methanesulfonyl-2-methoxyphenyl)amino]prop-1-yn-1-yl}-1-propyl-N- |
| | [(1R,4R)-4-(morpholin-4-yl)cyclohexyl]-1H-indol-4-amine |
| 879. | 2-{3-[(4-methanesulfonyl-2-methoxyphenyl)amino]prop-1-yn-1-yl}-1-(2-methylpropyl)- |
| | N-[(1R,4R)-4-(morpholin-4-yl)cyclohexyl]-1H-indol-4-amine |
| 880. | 1-(2,2-difluoroethyl)-2-{3-[(4-methanesulfonyl-2-methoxyphenyl)amino]prop-1-yn-1- |
| | yl}-N-[(1R,4R)-4-(morpholin-4-yl)cyclohexyl]-1H-indol-4-amine |
| 881. | 3-methoxy-4-((3-(4-(((1R,4R)-4-morpholinocyclohexyl)amino)-1-(2,2,2-trifluoroethyl)- |
| | 1H-indol-2-yl)prop-2-yn-1-yl)amino)benzenesulfonamide |
| 882. | 3-methoxy-4-((3-(4-(((1S,4S)-4-morpholinocyclohexyl)amino)-1-(2,2,2-trifluoroethyl)- |
| | 1H-indol-2-yl)prop-2-yn-1-yl)amino)benzenesulfonamide |
| 883. | N-ethyl-3-methoxy-4-{[3-(4-{[(1R,4R)-4-(morpholin-4-yl)cyclohexyl]amino}-1-(2,2,2- |
| | trifluoroethyl)-1H-indol-2-yl)prop-2-yn-1-yl]amino}benzamide |
| 884. | N-ethyl-3-methoxy-4-((3-(4-(((1S,4S)-4-morpholinocyclohexyl)amino)-1-(2,2,2- |
| | trifluoroethyl)-1H-indol-2-yl)prop-2-yn-1-yl)amino)benzamide |
| 885. | 2-(5-methanesulfonyl-2-{[3-(4-{[(1R,4R)-4-(morpholin-4-yl)cyclohexyl]amino}-1-(2,2,2- |
| | trifluoroethyl)-1H-indol-2-yl)prop-2-yn-1-yl]amino}phenoxy)acetonitrile |
| 886. | 2-(5-methanesulfonyl-2-{[3-(4-{[(1S,4S)-4-(morpholin-4-yl)cyclohexyl]amino}-1-(2,2,2- |
| | trifluoroethyl)-1H-indol-2-yl)prop-2-yn-1-yl]amino}phenoxy)acetonitrile |
| 887. | 3-methoxy-N-methyl-4-{[3-(4-{[(1R,4R)-4-(morpholin-4-yl)cyclohexyl]amino}-1-(2,2,2- |
| | trifluoroethyl)-1H-indol-2-yl)prop-2-yn-1-yl]amino}benzene-1-sulfonamide |
| 888. | 2-{3-[(4-methanesulfonyl-2-methoxyphenyl)amino]prop-1-yn-1-yl}-N-[(1R,4R)-4-(3- |
| | methanesulfonylazetidin-1-yl)cyclohexyl]-1-(2,2,2-trifluoroethyl)-1H-indol-4-amine |
| 889. | 2-(3-((2-methoxy-4-(methylsulfonyl)phenyl)amino)prop-1-yn-1-yl)-N-((1S,4S)-4-(3- |
| | (methylsulfonyl)azetidin-1-yl)cyclohexyl)-1-(2,2,2-trifluoroethyl)-1H-indol-4-amine |
| 890. | 3-methoxy-4-{[3-(4-{[(1R,4R)-4-(3-methanesulfonylazetidin-1-yl)cyclohexyl]amino}-1- |
| | (2,2,2-trifluoroethyl)-1H-indol-2-yl)prop-2-yn-1-yl]amino}benzene-1-sulfonamide |
| 891. | 3-methoxy-4-((3-(4-(((1S,4S)-4-(3-(methylsulfonyl)azetidin-1-yl)cyclohexyl)amino)-1- |
| | (2,2,2-trifluoroethyl)-1H-indol-2-yl)prop-2-yn-1-yl)amino)benzenesulfonamide |
| 892. | 3-methoxy-N-methyl-4-{[3-(4-{[(1R,4R)-4-(3-methanesulfonylazetidin-1- |
| | yl)cyclohexyl]amino}-1-(2,2,2-trifluoroethyl)-1H-indol-2-yl)prop-2-yn-1- |
| | yl]amino}benzamide |
| 893. | 3-methoxy-N-methyl-4-((3-(4-(((1S,4S)-4-(3-(methylsulfonyl)azetidin-1- |
| | yl)cyclohexyl)amino)-1-(2,2,2-trifluoroethyl)-1H-indol-2-yl)prop-2-yn-1- |
| | yl)amino)benzamide |
| 894. | 2-{3-[(4-methanesulfonyl-2-methoxyphenyl)amino]prop-1-yn-1-yl}-N-[(1R,4R)-4-{2- |
| | azaspiro[3.3]heptan-2-yl}cyclohexyl]-1-(2,2,2-trifluoroethyl)-1H-indol-4-amine |
| 895. | N-((1S,4S)-4-(2-azaspiro[3.3]heptan-2-yl)cyclohexyl)-2-(3-((2-methoxy-4- |
| | (methylsulfonyl)phenyl)amino)prop-1-yn-1-yl)-1-(2,2,2-trifluoroethyl)-1H-indol-4-amine |
| 896. | 3-methoxy-N-methyl-4-{[3-(4-{[(1R,4R)-4-{2-azaspiro[3.3]heptan-2- |
| | yl}cyclohexyl]amino}-1-(2,2,2-trifluoroethyl)-1H-indol-2-yl)prop-2-yn-1- |
| | yl]amino}benzamide |
| 897. | 4-((3-(4-(((1S,4S)-4-(2-azaspiro[3.3]heptan-2-yl)cyclohexyl)amino)-1-(2,2,2- |
| | trifluoroethyl)-1H-indol-2-yl)prop-2-yn-1-yl)amino)-3-methoxy-N-methylbenzamide |
| 898. | 3-methoxy-4-{[3-(4-{[(1R,4R)-4-{2-azaspiro[3.3]heptan-2-yl}cyclohexyl]amino}-1- |
| | (2,2,2-trifluoroethyl)-1H-indol-2-yl)prop-2-yn-1-yl]amino}benzene-1-sulfonamide |
| 899. | 4-((3-(4-(((1S,4S)-4-(2-azaspiro[3.3]heptan-2-yl)cyclohexyl)amino)-1-(2,2,2- |
| | trifluoroethyl)-1H-indol-2-yl)prop-2-yn-1-yl)amino)-3-methoxybenzenesulfonamide |
| 900. | 2-(3-((2-methoxy-4-(methylsulfonyl)phenyl)amino)prop-1-yn-1-yl)-N-(2- |
| | azaspiro[3.3]heptan-6-yl)-1-(2,2,2-trifluoroethyl)-1H-indol-4-amine |
| 901. | 4-((3-(4-((2-azaspiro[3.3]heptan-6-yl)amino)-1-(2,2,2-trifluoroethyl)-1H-indol-2-yl)prop- |
| | 2-yn-1-yl)amino)-3-methoxybenzenesulfonamide |
| 902. | 2-{3-[(4-methanesulfonyl-2-methoxyphenyl)amino]prop-1-yn-1-yl}-N-[(1R,4R)-4-{2- |
| | oxa-6-azaspiro[3.3]heptan-6-yl}cyclohexyl]-1-(2,2,2-trifluoroethyl)-1H-indol-4-amine |
| 903. | 2-{3-[(4-methanesulfonyl-2-methoxyphenyl)amino]prop-1-yn-1-yl}-N-[(1S,4S)-4-{2- |
| | oxa-6-azaspiro[3.3]heptan-6-yl}cyclohexyl]-1-(2,2,2-trifluoroethyl)-1H-indol-4-amine |
| 904. | 3-methoxy-4-{[3-(4-{[(1S,4S)-4-{2-oxa-6-azaspiro[3.3]heptan-6-yl}cyclohexyl]amino}- |
| | 1-(2,2,2-trifluoroethyl)-1H-indol-2-yl)prop-2-yn-1-yl]amino}benzene-1-sulfonamide |
| 905. | 3-methoxy-4-{[3-(4-{[(1R,4R)-4-{2-oxa-6-azaspiro[3.3]heptan-6-yl}cyclohexyl]amino}- |
| | 1-(2,2,2-trifluoroethyl)-1H-indol-2-yl)prop-2-yn-1-yl]amino}benzene-1-sulfonamide |
| 906. | N-(3-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzenesulfonyl)propanamide |
| 907. | N-(3-methoxy-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzenesulfonyl)propanamide |
| 908. | N-(3-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzenesulfonyl)acetamide |
| 909. | N-(3-methoxy-4-{[3-(4-{[(1S,4S)-4-}2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzenesulfonyl)acetamide |
| 910. | N-((1R,4R)-4-(2-oxa-6-azaspiro[3.3]heptan-6-yl)cyclohexyl)-2-(3-((2-(2-fluoroethoxy)-4- |
| | (methylsulfonyl)phenyl)amino)prop-1-yn-1-yl)-1-(2,2,2-trifluoroethyl)-1H-indol-4-amine |
| 911. | N-((1S,4S)-4-(2-oxa-6-azaspiro[3.3]heptan-6-yl)cyclohexyl)-2-(3-((2-(2-fluoroethoxy)-4- |
| | (methylsulfonyl)phenyl)amino)prop-1-yn-1-yl)-1-(2,2,2-trifluoroethyl)-1H-indol-4-amine |
| 912. | 3-methoxy-4-{[3-(4-{[(1R,4R)-4-{2-oxa-6-azaspiro[3.3]heptan-6-yl}cyclohexyl]amino}- |
| | 1-(2,2,2-trifluoroethyl)-1H-indol-2-yl)prop-2-yn-1-yl]amino}benzamide |
| 913. | 3-methoxy-4-{[3-(4-{[(1S,4S)-4-{2-oxa-6-azaspiro[3.3]heptan-6-yl}cyclohexyl]amino}- |
| | 1-(2,2,2-trifluoroethyl)-1H-indol-2-yl)prop-2-yn-1-yl]amino}benzamide |
| 914. | 5-methanesulfonyl-2-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenol |
| 915. | 5-methanesulfonyl-2-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenol |
| 916. | N-(3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)-N-(2-hydroxy-4- |
| | (methylsulfonyl)phenyl)propionamide |
| 917. | N-(3-(4-(((1R,4R)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)-N-(2-hydroxy-4- |
| | (methylsulfonyl)phenyl)propionamide |
| 918. | N-(2-hydroxy-4-methanesulfonylphenyl)-2-methyl-N-[3-(4-{[(1R,4R)-4-{2-oxa-6- |
| | azaspiro[3.3]heptan-6-yl}cyclohexyl]amino}-1-(2,2,2-trifluoroethyl)-1H-indol-2-yl)prop- |
| | 2-yn-1-yl]propanamide |
| 919. | N-(3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)-N-(2-hydroxy-4- |
| | (methylsulfonyl)phenyl)isobutyramide |
| 920. | 2-(3-{[4-methanesulfonyl-2-(2-methoxyethoxy)phenyl]amino}prop-1-yn-1-yl)-N- |
| | [(1R,4R)-4-{2-oxa-6-azaspiro[3.3]heptan-6-yl}cyclohexyl]-1-(2,2,2-trifluoroethyl)-1H- |
| | indol-4-amine |
| 921. | 2-(3-{[4-methanesulfonyl-2-(2-methoxyethoxy)phenyl]amino}prop-1-yn-1-yl)-N- |
| | [(1S,4S)-4-{2-oxa-6-azaspiro[3.3]heptan-6-yl}cyclohexyl]-1-(2,2,2-trifluoroethyl)-1H- |
| | indol-4-amine |
| 922. | 2-(5-methanesulfonyl-2-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenoxy)acetonitrile |
| 923. | 2-(5-methanesulfonyl-2-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenoxy)acetonitrile |
| 924. | 5-methanesulfonyl-2-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenyl 2-methylpropanoate |
| 925. | 2-(5-methanesulfonyl-2-([3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenoxy)ethan-1-ol |
| 926. | 2-(5-methanesulfonyl-2-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenoxy)ethan-1-ol |
| 927. | N-(3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)-N-(2-hydroxy-4- |
| | (methylsulfonyl)phenyl)acetamide |
| 928. | 5-methanesulfonyl-2-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenyl 2-methylpropanoate |
| 929. | 3-methoxy-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 930. | 3-methoxy-N-methyl-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 931. | 2-(3-{[4-methanesulfonyl-2-(2,2,2-trifluoroethoxy)phenyl]amino}prop-1-yn-1-yl)-N- |
| | [(1R,4R)-4-{2-oxa-6-azaspiro[3.3]heptan-6-yl}cyclohexyl]-1-(2,2,2-trifluoroethyl)-1H- |
| | indol-4-amine |
| 932. | 2-(3-{[4-methanesulfonyl-2-(2,2,2-trifluoroethoxy)phenyl]amino}prop-1-yn-1-yl)-N- |
| | [(1S,4S)-4-{2-oxa-6-azaspiro[3.3]heptan-6-yl}cyclohexyl]-1-(2,2,2-trifluoroethyl)-1H- |
| | indol-4-amine |
| 933. | 2-[3-(2-amino-4-methanesulfonylphenoxy)prop-1-yn-1-yl]-N-[(1S,4S)-4-{2-oxa-6- |
| | azaspiro[3.3]heptan-6-yl}cyclohexyl]-1-(2,2,2-trifluoroethyl)-1H-indol-4-amine |
| 934. | 2-{3-[(2-ethoxy-4-methanesulfonylphenyl)amino]prop-1-yn-1-yl}-N-[(1R,4R)-4-{2-oxa- |
| | 6-azaspiro[3.3]heptan-6-yl}cyclohexyl]-1-(2,2,2-trifluoroethyl)-1H-indol-4-amine |
| 935. | 2-{3-[(2-ethoxy-4-methanesulfonylphenyl)amino]prop-1-yn-1-yl}-N-[(1S,4S)-4-{2-oxa- |
| | 6-azaspiro[3.3]heptan-6-yl}cyclohexyl]-1-(2,2,2-trifluoroethyl)-1H-indol-4-amine |
| 936. | 3-(2-methoxyethoxy)-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 937. | 3-(2-methoxyethoxy)-N-methyl-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 938. | N-ethyl-3-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 939. | N-ethyl-3-methoxy-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 940. | 3-(2-fluoroethoxy)-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 941. | 3-(2-fluoroethoxy)-N-methyl-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 942. | 3-(cyanomethoxy)-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 943. | 3-(cyanomethoxy)-N-methyl-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 944. | 2-{3-[4-methanesulfonyl-2-(methylamino)phenoxy]prop-1-yn-1-yl}-N-[(1S,4S)-4-{2- |
| | oxa-6-azaspiro[3.3]heptan-6-yl}cyclohexyl]-1-(2,2,2-trifluoroethyl)-1H-indol-4-amine |
| 945. | 2-{3-[4-methanesulfonyl-2-(methylamino)phenoxy]prop-1-yn-1-yl}-N-[(1R,4R)-4-{2- |
| | oxa-6-azaspiro[3.3]heptan-6-yl}cyclohexyl]-1-(2,2,2-trifluoroethyl)-1H-indol-4-amine |
| 946. | 2-[3-(2-amino-4-methanesulfonylphenoxy)prop-1-yn-1-yl]-N-[(1R,4R)-4-{2-oxa-6- |
| | azaspiro[3.3]heptan-6-yl}cyclohexyl]-1-(2,2,2-trifluoroethyl)-1H-indol-4-amine |
| 947. | N-((1R,4R)-4-(2-oxa-6-azaspiro[3.3]heptan-6-yl)cyclohexyl)-2-(3-(2-amino-4- |
| | (methylsulfonyl)phenoxy)prop-1-yn-1-yl)-1-(2,2,2-trifluoroethyl)-1H-indol-4-amine |
| 948. | 3-hydroxy-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 949. | 3-methoxy-4-[(3-{4-[(4-{2-oxa-6-azaspiro[3.3]heptan-6-yl}cyclohexyl)amino]-1-(2,2,2- |
| | trifluoroethyl)-1H-indol-2-yl}prop-2-yn-1-yl)amino]benzonitrile |
| 950. | 3-methoxy-4-{[3-(4-{[(1S,4S)-4-{2-oxa-6-azaspiro[3.3]heptan-6-yl}cyclohexyl]amino}- |
| | 1-(2,2,2-trifluoroethyl)-1H-indol-2-yl)prop-2-yn-1-yl]amino}benzonitrile |
| 951. | 2-(3-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenyl)-2-methylpropanenitrile |
| 952. | 2-(4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-methoxyphenyl)-2- |
| | methylpropanenitrile |
| 953. | 3-(fluoromethoxy)-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 954. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-(fluoromethoxy)-N- |
| | methylbenzamide |
| 955. | 3-(2-cyanoethoxy)-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 956. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-(2-cyanoethoxy)-N- |
| | methylbenzamide |
| 957. | 2-(3-{[2-(fluoromethoxy)-4-methanesulfonylphenyl]amino}prop-1-yn-1-yl)-N-[(1R,4R)- |
| | 4-{2-oxa-6-azaspiro[3.3]heptan-6-yl}cyclohexyl]-1-(2,2,2-trifluoroethyl)-1H-indol-4- |
| | amine |
| 958. | N-((1S,4S)-4-(2-oxa-6-azaspiro[3.3]heptan-6-yl)cyclohexyl)-2-(3-((2-(fluoromethoxy)-4- |
| | (methylsulfonyl)phenyl)amino)prop-1-yn-1-yl)-1-(2,2,2-trifluoroethyl)-1H-indol-4-amine |
| 959. | 3-(cyanomethoxy)-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzene-1-sulfonamide |
| 960. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3- |
| | (cyanomethoxy)benzenesulfonamide |
| 961. | 3-(fluoromethoxy)-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzene-1-sulfonamide |
| 962. | 3-(fluoromethoxy)-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzene-1-sulfonamide |
| 963. | 4-((3-(4-(((1R,4R)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-methoxy-N- |
| | methylbenzenesulfonamide |
| 964. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-methoxy-N- |
| | methylbenzenesulfonamide |
| 965. | N-((1R,4R)-4-(2-oxa-6-azaspiro[3.3]heptan-6-yl)cyclohexyl)-2-(3-((4-chloro-2- |
| | methoxyphenyl)amino)prop-1-yn-1-yl)-1-(2,2,2-trifluoroethyl)-1H-indol-4-amine |
| 966. | N-((1S,4S)-4-(2-oxa-6-azaspiro[3.3]heptan-6-yl)cyclohexyl)-2-(3-((4-chloro-2- |
| | methoxyphenyl)amino)prop-1-yn-1-yl)-1-(2,2,2-trifluoroethyl)-1H-indol-4-amine |
| 967. | 4-((3-(4-(((1R,4R)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-methoxy-N,N- |
| | dimethylbenzenesulfonamide |
| 968. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-methoxy-N,N- |
| | dimethylbenzenesulfonamide |
| 969. | N-((1R,4R)-4-(2-oxa-6-azaspiro[3.3]heptan-6-yl)cyclohexyl)-2-(3-((4-(ethylsulfonyl)-2- |
| | methoxyphenyl)amino)prop-1-yn-1-yl)-1-(2,2,2-trifluoroethyl)-1H-indol-4-amine |
| 970. | N-((1S,4S)-4-(2-oxa-6-azaspiro[3.3]heptan-6-yl)cyclohexyl)-2-(3-((4-(ethylsulfonyl)-2- |
| | methoxyphenyl)amino)prop-1-yn-1-yl)-1-(2,2,2-trifluoroethyl)-1H-indol-4-amine |
| 971. | N-((1R,4R)-4-(2-oxa-6-azaspiro[3.3]heptan-6-yl)cyclohexyl)-2-(3-(((3S,4R)-3- |
| | methoxytetrahydro-2H-pyran-4-yl)amino)prop-1-yn-1-yl)-1-(2,2,2-trifluoroethyl)-1H- |
| | indol-4-amine |
| 972. | N-((1S,4S)-4-(2-oxa-6-azaspiro[3.3]heptan-6-yl)cyclohexyl)-2-(3-(((3S,4R)-3- |
| | methoxytetrahydro-2H-pyran-4-yl)amino)prop-1-yn-1-yl)-1-(2,2,2-trifluoroethyl)-1H- |
| | indol-4-amine |
| 973. | 2-fluoro-5-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 974. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-2-fluoro-5-methoxybenzamide |
| 975. | 2-fluoro-5-methoxy-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 976. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-2-fluoro-5-methoxy-N- |
| | methylbenzamide |
| 977. | 2-(3-{[2-methoxy-4-(trifluoromethyl)phenyl]amino}prop-1-yn-1-yl)-N-[(1R,4R)-4-{2- |
| | oxa-6-azaspiro[3.3]heptan-6-yl}cyclohexyl]-1-(2,2,2-trifluoroethyl)-1H-indol-4-amine |
| 978. | N-((1S,4S)-4-(2-oxa-6-azaspiro[3.3]heptan-6-yl)cyclohexyl)-2-(3-((2-methoxy-4- |
| | (trifluoromethyl)phenyl)amino)prop-1-yn-1-yl)-1-(2,2,2-trifluoroethyl)-1H-indol-4-amine |
| 979. | 4-((3-(4-(((1R,4R)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-methoxybenzoic acid |
| 980. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-methoxybenzoic acid |
| 981. | 2-fluoro-5-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzoic acid |
| 982. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-2-fluoro-5-methoxybenzoic acid |
| 983. | 2-{3-[(4-methanesulfonyl-2-methylphenyl)amino]prop-1-yn-1-yl}-N-[(1R,4R)-4-{2-oxa- |
| | 6-azaspiro[3.3]heptan-6-yl}cyclohexyl]-1-(2,2,2-trifluoroethyl)-1H-indol-4-amine |
| 984. | N-((1S,4S)-4-(2-oxa-6-azaspiro[3.3]heptan-6-yl)cyclohexyl)-2-(3-((2-methyl-4- |
| | (methylsulfonyl)phenyl)amino)prop-1-yn-1-yl)-1-(2,2,2-trifluoroethyl)-1H-indol-4-amine |
| 985. | 2-(3-{[4-methanesulfonyl-2-(trifluoromethoxy)phenyl]amino}prop-1-yn-1-yl)-N- |
| | [(1R,4R)-4-{2-oxa-6-azaspiro[3.3]heptan-6-yl}cyclohexyl]-1-(2,2,2-trifluoroethyl)-1H- |
| | indol-4-amine |
| 986. | N-((1S,4S)-4-(2-oxa-6-azaspiro[3.3]heptan-6-yl)cyclohexyl)-2-(3-((4-(methylsulfonyl)-2- |
| | (trifluoromethoxy)phenyl)amino)prop-1-yn-1-yl)-1-(2,2,2-trifluoroethyl)-1H-indol-4- |
| | amine |
| 987. | 2-(3-{[4-methanesulfonyl-2-(trifluoromethyl)phenyl]amino}prop-1-yn-1-yl)-N-[(1R,4R)- |
| | 4-{2-oxa-6-azaspiro[3.3]heptan-6-yl}cyclohexyl]-1-(2,2,2-trifluoroethyl)-1H-indol-4- |
| | amine |
| 988. | N-((1S,4S)-4-(2-oxa-6-azaspiro[3.3]heptan-6-yl)cyclohexyl)-2-(3-((4-(methylsulfonyl)-2- |
| | (trifluoromethyl)phenyl)amino)prop-1-yn-1-yl)-1-(2,2,2-trifluoroethyl)-1H-indol-4-amine |
| 989. | 2-{3-[(2-chloro-4-methanesulfonylphenyl)amino]prop-1-yn-1-yl}-N-[(1R,4R)-4-{2-oxa- |
| | 6-azaspiro[3.3]heptan-6-yl}cyclohexyl]-1-(2,2,2-trifluoroethyl)-1H-indol-4-amine |
| 990. | N-((1S,4S)-4-(2-oxa-6-azaspiro[3.3]heptan-6-yl)cyclohexyl)-2-(3-((2-chloro-4- |
| | (methylsulfonyl)phenyl)amino)prop-1-yn-1-yl)-1-(2,2,2-trifluoroethyl)-1H-indol-4-amine |
| 991. | 3-(3-(4-(((1R,4R)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)-6-(methylsulfonyl)benzo[d]oxazol-2(3H)-one |
| 992. | 3-(3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)-6-(methylsulfonyl)benzo[d]oxazol-2(3H)-one |
| 993. | 2-{3-[(4-methanesulfonyl-2-methoxyphenyl)amino]prop-1-yn-1-yl}-N-[(1S,4S)-4-{7- |
| | oxa-2-azaspiro[3.5]nonan-2-yl]cyclohexyl]-1-(2,2,2-trifluoroethyl)-1H-indol-4-amine |
| 994. | 2-{3-[(4-methanesulfonyl-2-methoxyphenyl)amino]prop-1-yn-1-yl}-N-[(1R,4R)-4-{7- |
| | oxa-2-azaspiro[3.5]nonan-2-yl]cyclohexyl]-1-(2,2,2-trifluoroethyl)-1H-indol-4-amine |
| 995. | 3-methoxy-N-methyl-4-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzamide |
| 996. | 3-methoxy-N-methyl-4-{[3-(4-{[(1S,4S)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzamide |
| 997. | 3-methoxy-4-{[3-(4-{[(1R,4R)-4-{7-oxa-2-azaspiro[3.5]nonan-2-yl}cyclohexyl]amino}- |
| | 1-(2,2,2-trifluoroethyl)-1H-indol-2-yl)prop-2-yn-1-yl]amino}benzene-1-sulfonamide |
| 998. | 3-methoxy-4-{[3-(4-{[(1S,4S)-4-{7-oxa-2-azaspiro[3.5]nonan-2-yl}cyclohexyl]amino}- |
| | 1-(2,2,2-trifluoroethyl)-1H-indol-2-yl)prop-2-yn-1-yl]amino}benzene-1-sulfonamide |
| 999. | 3-(cyanomethoxy)-4-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzene-1-sulfonamide |
| 1000. | 3-(cyanomethoxy)-4-{[3-(4-{[(1S,4S)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzene-1-sulfonamide |
| 1001. | 2-(3-methoxy-4-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}phenyl)-2-methylpropanenitrile |
| 1002. | 2-(4-((3-(4-(((1S,4S)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxyphenyl)-2- |
| | methylpropanenitrile |
| 1003. | 2-(5-methanesulfonyl-2-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}phenoxy)acetonitrile |
| 1004. | 2-(5-methanesulfonyl-2-{[3-(4-([(1S,4S)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}phenoxy)acetonitrile |
| 1005. | N-((4-((3-(4-(((1R,4R)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3- |
| | methoxyphenyl)sulfonyl)acetamide |
| 1006. | N-((4-((3-(4-(((1S,4S)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3- |
| | methoxyphenyl)sulfonyl)acetamide |
| 1007. | N-((4-((3-(4-(((1R,4R)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxyphenyl)sulfonyl)-2- |
| | aminoacetamide |
| 1008. | N-((4-((3-(4-(((1S,4S)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxyphenyl)sulfonyl)-2- |
| | aminoacetamide |
| 1009. | methyl 2-(5-methanesulfonyl-2-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}phenoxy)acetate |
| 1010. | methyl 2-(2-((3-(4-(((1S,4S)-4-(7-oxa-2-azaspiro[3.5]nonan-2-yl)cyclohexyl)amino)-1- |
| | (2,2,2-trifluoroethyl)-1H-indol-2-yl)prop-2-yn-1-yl)amino)-5- |
| | (methylsulfonyl)phenoxy)acetate |
| 1011. | 2-(5-methanesulfonyl-2-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}phenoxy)acetic acid |
| 1012. | 2-(2-((3-(4-(((1S,4S)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-5-(methylsulfonyl)phenoxy)acetic acid |
| 1013. | 2-{3-[(4-methanesulfonyl-2-methoxyphenyl)amino]prop-1-yn-1-yl}-N-[(1R,4R)-4-{2- |
| | oxa-7-azaspiro[3.5]nonan-7-yl}cyclohexyl]-1-(2,2,2-trifluoroethyl)-1H-indol-4-amine |
| 1014. | 2-(3-{[4-(ethanesulfonyl)-2-methoxyphenyl]amino}prop-1-yn-1-yl)-N-[(1R,4R)-4-{7- |
| | oxa-2-azaspiro[3.5]nonan-2-yl}cyclohexyl]-1-(2,2,2-trifluoroethyl)-1H-indol-4-amine |
| 1015. | 2-(3-{[4-(ethanesulfonyl)-2-methoxyphenyl]amino}prop-1-yn-1-yl)-N-[(1S,4S)-4-{7-oxa- |
| | 2-azaspiro[3.5]nonan-2-yl}cyclohexyl]-1-(2,2,2-trifluoroethyl)-1H-indol-4-amine |
| 1016. | 3-(fluoromethoxy)-4-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzene-1-sulfonamide |
| 1017. | 3-(fluoromethoxy)-4-{[3-(4-{[(1S,4S)-4-{7-oxa-2-azaspiro[3,5]nonan-2- |
| | yl}cyclohexyl]amino}-1-(2,2,2-trifluoroethyl)-1H-indol-2-yl)prop-2-yn-1- |
| | yl]amino}benzene-1-sulfonamide |
| 1018. | 3-methoxy-N-methyl-4-{[3-(4-{[(1R,4R)-4-{6-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzamide |
| 1019. | 4-((3-(4-(((1S,4S)-4-(6-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxy-N-methylbenzamide |
| 1020. | 3-methoxy-4-{[3-(4-{[(1R,4R)-4-{6-oxa-2-azaspiro[3.5]nonan-2-yl}cyclohexyl]amino}- |
| | 1-(2,2,2-trifluoroethyl)-1H-indol-2-yl)prop-2-yn-1-yl]amino}benzene-1-sulfonamide |
| 1021. | 4-((3-(4-(((1S,4S)-4-(6-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxybenzenesulfonamide |
| 1022. | 2-{3-[(4-methanesulfonyl-2-methoxyphenyl)amino]prop-1-yn-1-yl}-N-[(1R,4R)-4-{6- |
| | oxa-2-azaspiro[3.5]nonan-2-yl}cyclohexyl]-1-(2,2,2-trifluoroethyl)-1H-indol-4-amine |
| 1023. | N-((1S,4S)-4-(6-oxa-2-azaspiro[3.5]nonan-2-yl)cyclohexyl)-2-(3-((2-methoxy-4- |
| | (methylsulfonyl)phenyl)amino)prop-1-yn-1-yl)-1-(2,2,2-trifluoroethyl)-1H-indol-4-amine |
| 1024. | N-((1R,4R)-4-(7-oxa-2-azaspiro[3.5]nonan-2-yl)cyclohexyl)-2-(3-((2-(fluoromethoxy)-4- |
| | (methylsulfonyl)phenyl)amino)prop-1-yn-1-yl)-1-(2,2,2-trifluoroethyl)-1H-indol-4-amine |
| 1025. | N-((1S,4S)-4-(7-oxa-2-azaspiro[3.5]nonan-2-yl)cyclohexyl)-2-(3-((2-(fluoromethoxy)-4- |
| | (methylsulfonyl)phenyl)amino)prop-1-yn-1-yl)-1-(2,2,2-trifluoroethyl)-1H-indol-4-amine |
| 1026. | 4-(3-(4-(((1R,4R)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)-7-(methylsulfonyl)-2H- |
| | benzo[b][1,4]oxazin-3(4H)-one |
| 1027. | 4-((3-(4-(((1R,4R)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxy-N- |
| | methylbenzenesulfonamide |
| 1028. | 4-((3-(4-(((1S,4S)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxy-N- |
| | methylbenzenesulfonamide |
| 1029. | N-((1R,4R)-4-(7-oxa-2-azaspiro[3.5]nonan-2-yl)cyclohexyl)-2-(3-((2-ethoxy-4- |
| | (methylsulfonyl)phenyl)amino)prop-1-yn-1-yl)-1-(2,2,2-trifluoroethyl)-1H-indol-4-amine |
| 1030. | N-((1S,4S)-4-(7-oxa-2-azaspiro[3.5]nonan-2-yl)cyclohexyl)-2-(3-((2-ethoxy-4- |
| | (methylsulfonyl)phenyl)amino)prop-1-yn-1-yl)-1-(2,2,2-trifluoroethyl)-1H-indol-4-amine |
| 1031. | 3-methoxy-4-{[3-(4-{[(1R,4R)-4-{7-oxa-2-azaspiro[3.5]nonan-2-yl}cyclohexyl]amino}- |
| | 1-(2,2,2-trifluoroethyl)-1H-indol-2-yl)prop-2-yn-1-yl]amino}benzamide |
| 1032. | 4-((3-(4-(((1S,4S)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxybenzamide |
| 1033. | 3-methoxy-4-{[3-(4-{[(1R,4R)-4-{6-oxa-2-azaspiro[3.5]nonan-2-yl}cyclohexyl]amino}- |
| | 1-(2,2,2-trifluoroethyl)-1H-indol-2-yl)prop-2-yn-1-yl]amino}benzamide |
| 1034. | 4-((3-(4-(((1S,4S)-4-(6-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxybenzamide |
| 1035. | 2-fluoro-5-methoxy-N-methyl-4-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzamide |
| 1036. | 2-(3-((2-methoxy-4-(methylsulfonyl)phenyl)amino)prop-1-yn-1-yl)-N-((1R,4R)-4-(3- |
| | methoxypyrrolidin-1-yl)cyclohexyl)-1-(2,2,2-trifluoroethyl)-1H-indol-4-amine |
| 1037. | 2-(3-((2-methoxy-4-(methylsulfonyl)phenyl)amino)prop-1-yn-1-yl)-N-((1S,4S)-4-(3- |
| | methoxypyrrolidin-1-yl)cyclohexyl)-1-(2,2,2-trifluoroethyl)-1H-indol-4-amine |
| 1038. | 3-methoxy-4-((3-(4-(((1R,4R)-4-(3-methoxypyrrolidin-1-yl)cyclohexyl)amino)-1-(2,2,2- |
| | trifluoroethyl)-1H-indol-2-yl)prop-2-yn-1-yl)amino)benzenesulfonamide |
| 1039. | 3-methoxy-4-((3-(4-(((1S,4S)-4-(3-methoxypyrrolidin-1-yl)cyclohexyl)amino)-1-(2,2,2- |
| | trifluoroethyl)-1H-indol-2-yl)prop-2-yn-1-yl)amino)benzenesulfonamide |
| 1040. | 2-(3-{[2-(fluoromethoxy)-4-methanesulfonylphenyl]amino}prop-1-yn-1-yl)-N-[(1R,4R)- |
| | 4-(morpholin-4-yl)cyclohexyl]-1-(2,2,2-trifluoroethyl)-1H-indol-4-amine |
| 1041. | 2-(3-{[2-(fluoromethoxy)-4-methanesulfonylphenyl]amino}prop-1-yn-1-yl)-N-[(1S,4S)- |
| | 4-(morpholin-4-yl)cyclohexyl]-1-(2,2,2-trifluoroethyl)-1H-indol-4-amine |
| 1042. | 3-(cyanomethoxy)-4-{[3-(4-{[(1R,4R)-4-(morpholin-4-yl)cyclohexyl]amino}-1-(2,2,2- |
| | trifluoroethyl)-1H-indol-2-yl)prop-2-yn-1-yl]amino}benzene-1-sulfonamide |
| 1043. | 3-(cyanomethoxy)-4-{[3-(4-{[(1S,4S)-4-(morpholin-4-yl)cyclohexyl]amino}-1-(2,2,2- |
| | trifluoroethyl)-1H-indol-2-yl)prop-2-yn-1-yl]amino}benzene-1-sulfonamide |
| 1044. | 3-(fluoromethoxy)-4-{[3-(4-{[(1R,4R)-4-(morpholin-4-yl)cyclohexyl]amino}-1-(2,2,2- |
| | trifluoroethyl)-1H-indol-2-yl)prop-2-yn-1-yl]amino}benzene-1-sulfonamide |
| 1045. | 3-(fluoromethoxy)-4-{[3-(4-{[(1S,4S)-4-(morpholin-4-yl)cyclohexyl]amino}-1-(2,2,2- |
| | trifluoroethyl)-1H-indol-2-yl)prop-2-yn-1-yl]amino}benzene-1-sulfonamide |
| 1046. | 2-(3-((2-ethoxy-4-(methylsulfonyl)phenyl)amino)prop-1-yn-1-yl)-N-((1R,4R)-4- |
| | morpholinocyclohexyl)-1-(2,2,2-trifluoroethyl)-1H-indol-4-amine |
| 1047. | 2-(3-((2-ethoxy-4-(methylsulfonyl)phenyl)amino)prop-1-yn-1-yl)-N-((1S,4S)-4- |
| | morpholinocyclohexyl)-1-(2,2,2-trifluoroethyl)-1H-indol-4-amine |
| 1048. | 2-{3-[(4-methanesulfonyl-2-methoxyphenyl)amino]prop-1-yn-1-yl}-N-[(1R,4R)-4-(3- |
| | methoxypiperidin-1-yl)cyclohexyl]-1-(2,2,2-trifluoroethyl)-1H-indol-4-amine |
| 1049. | 2-(3-((2-methoxy-4-(methylsulfonyl)phenyl)amino)prop-1-yn-1-yl)-N-((1S,4S)-4-(3- |
| | methoxypiperidin-1-yl)cyclohexyl)-1-(2,2,2-trifluoroethyl)-1H-indol-4-amine |
| 1050. | 2-{3-[(4-methanesulfonyl-2-methoxyphenyl)amino]prop-1-yn-1-yl}-N-[(1R,4R)-4-(4- |
| | methoxypiperidin-1-yl)cyclohexyl]-1-(2,2,2-trifluoroethyl)-1H-indol-4-amine |
| 1051. | 2-(3-((2-methoxy-4-(methylsulfonyl)phenyl)amino)prop-1-yn-1-yl)-N-((1S,4S)-4-(4- |
| | methoxypiperidin-1-yl)cyclohexyl)-1-(2,2,2-trifluoroethyl)-1H-indol-4-amine |
| 1052. | 3-methoxy-N-methyl-4-{[3-(4-{[(1R,4R)-4-(4-methoxypiperidin-1- |
| | yl)cyclohexyl]amino}-1-(2,2,2-trifluoroethyl)-1H-indol-2-yl)prop-2-yn-1- |
| | yl]amino}benzamide |
| 1053. | 3-methoxy-4-((3-(4-(((1S,4S)-4-(4-methoxypiperidin-1-yl)cyclohexyl)amino)-1-(2,2,2- |
| | trifluoroethyl)-1H-indol-2-yl)prop-2-yn-1-yl)amino)-N-methylbenzamide |
| 1054. | 3-methoxy-4-{[3-(4-{[(1R,4R)-4-(4-methoxypiperidin-1-yl)cyclohexyl]amino}-1-(2,2,2- |
| | trifluoroethyl)-1H-indol-2-yl)prop-2-yn-1-yl]amino}benzene-1-sulfonamide |
| 1055. | 3-methoxy-4-((3-(4-(((1S,4S)-4-(4-methoxypiperidin-1-yl)cyclohexyl)amino)-1-(2,2,2- |
| | trifluoroethyl)-1H-indol-2-yl)prop-2-yn-1-yl)amino)benzenesulfonamide |
| 1056. | 3-methoxy-N-methyl-4-{[3-(4-{[(1R,4R)-4-(3-methoxypiperidin-1- |
| | yl)cyclohexyl]amino}-1-(2,2,2-trifluoroethyl)-1H-indol-2-yl)prop-2-yn-1- |
| | yl]amino}benzamide |
| 1057. | 3-methoxy-4-((3-(4-(((1S,4S)-4-(3-methoxypiperidin-1-yl)cyclohexyl)amino)-1-(2,2,2- |
| | trifluoroethyl)-1H-indol-2-yl)prop-2-yn-1-yl)amino)-N-methylbenzamide |
| 1058. | 3-methoxy-4-{[3-(4-{[(1R,4R)-4-(3-methoxypiperidin-1-yl)cyclohexyl]amino}-1-(2,2,2- |
| | trifluoroethyl)-1H-indol-2-yl)prop-2-yn-1-yl]amino}benzene-1-sulfonamide |
| 1059. | 3-methoxy-4-((3-(4-(((1S,4S)-4-(3-methoxypiperidin-1-yl)cyclohexyl)amino)-1-(2,2,2- |
| | trifluoroethyl)-1H-indol-2-yl)prop-2-yn-1-yl)amino)benzenesulfonamide |
| 1060. | 3-methoxy-4-((3-(4-(((1R,4R)-4-morpholinocyclohexyl)amino)-1-(2,2,2-trifluoroethyl)- |
| | 1H-indol-2-yl)prop-2-yn-1-yl)amino)benzenesulfonamide |
| 1061. | 3-methoxy-4-((3-(4-(((1S,4S)-4-morpholinocyclohexyl)amino)-1-(2,2,2-trifluoroethyl)- |
| | 1H-indol-2-yl)prop-2-yn-1-yl)amino)benzenesulfonamide |
| 1062. | 4-[(1R,4R)-4-[(2-{3-[(4-methanesulfonyl-2-methoxyphenyl)amino]prop-1-yn-1-yl}-1- |
| | (2,2,2-trifluoroethyl)-1H-indol-4-yl)amino]cyclohexyl]-1λ⁶-thiomorpholine-1,1-dione |
| 1063. | 4-((1S,4S)-4-((2-(3-((2-methoxy-4-(methylsulfonyl)phenyl)amino)prop-1-yn-1-yl)-1- |
| | (2,2,2-trifluoroethyl)-1H-indol-4-yl)amino)cyclohexyl)thiomorpholine 1,1-dioxide |
| 1064. | 4-[(1R,4R)-4-[(2-{3-[(4-methanesulfonyl-2-methoxyphenyl)amino]prop-1-yn-1-yl}-1- |
| | (2,2,2-trifluoroethyl)-1H-indol-4-yl)amino]cyclohexyl]-1λ⁴-thiomorpholin-1-one |
| 1065. | 4-[(1S,4S)-4-[(2-{3-[(4-methanesulfonyl-2-methoxyphenyl)amino]prop-1-yn-1-yl}-1- |
| | (2,2,2-trifluoroethyl)-1H-indol-4-yl)amino]cyclohexyl]-1λ⁴-thiomorpholin-1-one |
| 1066. | 4-((3-(4-(((1R,4R)-4-(1,1-dioxidothiomorpholino)cyclohexyl)amino)-1-(2,2,2- |
| | trifluoroethyl)-1H-indol-2-yl)prop-2-yn-1-yl)amino)-3-methoxy-N-methylbenzamide |
| 1067. | 4-((3-(4-(((1S,4S)-4-(1,1-dioxidothiomorpholino)cyclohexyl)amino)-1-(2,2,2- |
| | trifluoroethyl)-1H-indol-2-yl)prop-2-yn-1-yl)amino)-3-methoxy-N-methylbenzamide |
| 1068. | 4-((1S,4S)-4-((2-(3-((2-methoxy-4-(methylsulfonyl)phenyl)amino)prop-1-yn-1-yl)-1- |
| | (2,2,2-trifluoroethyl)-1H-indol-4-yl)amino)cyclohexyl)thiomorpholine 1-oxide |
| 1069. | 3-methoxy-N-methyl-4-{[3-(4-{[(1R,4R)-4-(1-oxo-1λ⁴-thiomorpholin-4- |
| | yl)cyclohexyl]amino}-1-(2,2,2-trifluoroethyl)-1H-indol-2-yl)prop-2-yn-1- |
| | yl]amino}benzamide |
| 1070. | 3-methoxy-4-{[3-(4-{[(1R,4R)-4-(1-oxo-1λ⁴-thiomorpholin-4-yl)cyclohexyl]amino}-1- |
| | (2,2,2-trifluoroethyl)-1H-indol-2-yl)prop-2-yn-1-yl]amino}benzene-1-sulfonamide |
| 1071. | 3-methoxy-4-((3-(4-(((1S,4S)-4-(1-oxidothiomorpholino)cyclohexyl)amino)-1-(2,2,2- |
| | trifluoroethyl)-1H-indol-2-yl)prop-2-yn-1-yl)amino)benzenesulfonamide |
| 1072. | 3-methoxy-4-{[3-(4-{[(1R,4R)-4-(1,1-dioxo-1λ⁶-thiomorpholin-4-yl)cyclohexyl]amino}- |
| | 1-(2,2,2-trifluoroethyl)-1H-indol-2-yl)prop-2-yn-1-yl]amino}benzene-1-sulfonamide |
| 1073. | 4-((3-(4-(((1S,4S)-4-(1,1-dioxidothiomorpholino)cyclohexyl)amino)-1-(2,2,2- |
| | trifluoroethyl)-1H-indol-2-yl)prop-2-yn-1-yl)amino)-3-methoxybenzenesulfonamide |
| 1074. | 2-{3-[(4-methanesulfonyl-2-methoxyphenyl)amino]prop-1-yn-1-yl}-N-[(1S,4S)-4-{2- |
| | oxa-7-azaspiro[3.5]nonan-7-yl}cyclohexyl]-1-(2,2,2-trifluoroethyl)-1H-indol-4-amine |
| 1075. | 3-methoxy-N-methyl-4-{[3-(4-{[(1R,4R)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzamide |
| 1076. | 3-methoxy-N-methyl-4-{[3-(4-{[(1S,4S)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzamide |
| 1077. | 3-methoxy-4-{[3-(4-{[(1R,4R)-4-{2-oxa-7-azaspiro[3.5]nonan-7-yl}cyclohexyl]amino}- |
| | 1-(2,2,2-trifluoroethyl)-1H-indol-2-yl)prop-2-yn-1-yl]amino}benzene-1-sulfonamide |
| 1078. | 4-((3-(4-(((1S,4S)-4-(2-oxa-7-azaspiro[3.5]nonan-7-yl)cyclohexyl)amino)-1-(2,2,2- |
| | trifluoroethyl)-1H-indol-2-yl)prop-2-yn-1-yl)amino)-3-methoxybenzenesulfonamide |
| 1079. | 3-(cyanomethoxy)-4-][3-(4-{[(1R,4R)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1080. | 3-(cyanomethoxy)-4-{[3-(4-{[(1S,4S)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1081. | 3-(fluoromethoxy)-4-{[3-(4-{[(1R,4R)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1082. | 3-(fluoromethoxy)-4-{[3-(4-{[(1S,4S)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1083. | 2-{3-[(4-methanesulfonyl-2-methoxyphenyl)amino]prop-1-yn-1-yl}-N-[(1R,4R)-4-{1- |
| | oxa-7-azaspiro[3.5]nonan-7-yl}cyclohexyl]-1-(2,2,2-trifluoroethyl)-1H-indol-4-amine |
| 1084. | N-((1S,4S)-4-(1-oxa-7-azaspiro[3.5]nonan-7-yl)cyclohexyl)-2-(3-((2-methoxy-4- |
| | (methylsulfonyl)phenyl)amino)prop-1-yn-1-yl)-1-(2,2,2-trifluoroethyl)-1H-indol-4-amine |
| 1085. | 3-methoxy-N-methyl-4-{[3-(4-{[(1R,4R)-4-{1-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzamide |
| 1086. | 4-((3-(4-(((1S,4S)-4-(1-oxa-7-azaspiro[3.5]nonan-7-yl)cyclohexyl)amino)-1-(2,2,2- |
| | trifluoroethyl)-1H-indol-2-yl)prop-2-yn-1-yl)amino)-3-methoxy-N-methylbenzamide |
| 1087. | 3-methoxy-4-{[3-(4-{[(1R,4R)-4-{1-oxa-7-azaspiro[3.5]nonan-7-yl}cyclohexyl]amino}- |
| | 1-(2,2,2-trifluoroethyl)-1H-indol-2-yl)prop-2-yn-1-yl]amino}benzene-1-sulfonamide |
| 1088. | 4-((3-(4-(((1S,4S)-4-(1-oxa-7-azaspiro[3.5]nonan-7-yl)cyclohexyl)amino)-1-(2,2,2- |
| | trifluoroethyl)-1H-indol-2-yl)prop-2-yn-1-yl)amino)-3-methoxybenzenesulfonamide |
| 1089. | 2-(5-methanesulfonyl-2-{[3-(4-{[(1R,4R)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}phenoxy)acetonitrile |
| 1090. | 2-(5-methanesulfonyl-2-{[3-(4-{[(1S,4S)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}phenoxy)acetonitrile |
| 1091. | 2-(3-{[2-(fluoromethoxy)-4-methanesulfonylphenyl]amino}prop-1-yn-1-yl)-N-[(1R,4R)- |
| | 4-{2-oxa-7-azaspiro[3.5]nonan-7-yl}cyclohexyl]-1-(2,2,2-trifluoroethyl)-1H-indol-4- |
| | amine |
| 1092. | 2-(3-{[2-(fluoromethoxy)-4-methanesulfonylphenyl]amino}prop-1-yn-1-yl)-N-[(1S,4S)- |
| | 4-{2-oxa-7-azaspiro[3.5]nonan-7-yl}cyclohexyl]-1-(2,2,2-trifluoroethyl)-1H-indol-4- |
| | amine |
| 1093. | N-((1R,4R)-4-(2-oxa-7-azaspiro[3.5]nonan-7-yl)cyclohexyl)-2-(3-((2-ethoxy-4- |
| | (methylsulfonyl)phenyl)amino)prop-1-yn-1-yl)-1-(2,2,2-trifluoroethyl)-1H-indol-4-amine |
| 1094. | N-((1S,4S)-4-(2-oxa-7-azaspiro[3.5]nonan-7-yl)cyclohexyl)-2-(3-((2-ethoxy-4- |
| | (methylsulfonyl)phenyl)amino)prop-1-yn-1-yl)-1-(2,2,2-trifluoroethyl)-1H-indol-4-amine |
| 1095. | 3-methoxy-N-methyl-4-{[3-(4-{[(1R,4R)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1096. | N-((1R,4R)-4-(2-oxa-7-azaspiro[3.5]nonan-7-yl)cyclohexyl)-2-(3-((4-(ethylsulfonyl)-2- |
| | methoxyphenyl)amino)prop-1-yn-1-yl)-1-(2,2,2-trifluoroethyl)-1H-indol-4-amine |
| 1097. | N-((1S,4S)-4-(2-oxa-7-azaspiro[3.5]nonan-7-yl)cyclohexyl)-2-(3-((4-(ethylsulfonyl)-2- |
| | methoxyphenyl)amino)prop-1-yn-1-yl)-1-(2,2,2-trifluoroethyl)-1H-indol-4-amine |
| 1098. | 2-{3-[(4-methanesulfonyl-2-methoxyphenyl)amino]prop-1-yn-1-yl}-N-[(1R,4R)-4-{6- |
| | oxa-3-azabicyclo[3.1.1]heptan-3-yl}cyclohexyl]-1-(2,2,2-trifluoroethyl)-1H-indol-4- |
| | amine |
| 1099. | 2-{3-[(4-methanesulfonyl-2-methoxyphenyl)amino]prop-1-yn-1-yl}-N-[(1S,4S)-4-{6- |
| | oxa-3-azabicyclo[3.1.1]heptan-3-yl}cyclohexyl]-1-(2,2,2-trifluoroethyl)-1H-indol-4- |
| | amine |
| 1100. | 3-methoxy-4-{[3-(4-{[(1R,4R)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}benzene-1-sulfonamide |
| 1101. | 3-methoxy-4-{[3-(4-{[(1S,4S)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}benzene-1-sulfonamide |
| 1102. | 3-methoxy-N-methyl-4-{[3-(4-{[(1R,4R)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}benzamide |
| 1103. | 3-methoxy-N-methyl-4-{[3-(4-{[(1S,4S)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}benzamide |
| 1104. | 2-{3-[(2-ethoxy-4-methanesulfonylphenyl)amino]prop-1-yn-1-yl}-N-[(1R,4R)-4-{6-oxa- |
| | 3-azabicyclo[3.1.1]heptan-3-yl}cyclohexyl]-1-(2,2,2-trifluoroethyl)-1H-indol-4-amine |
| 1105. | 2-{3-[(2-ethoxy-4-methanesulfonylphenyl)amino]prop-1-yn-1-yl}-N-[(1S,4S)-4-{6-oxa- |
| | 3-azabicyclo[3.1.1]heptan-3-yl}cyclohexyl]-1-(2,2,2-trifluoroethyl)-1H-indol-4-amine |
| 1106. | N-ethyl-3-methoxy-4-{[3-(4-{[(1R,4R)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}benzamide |
| 1107. | 2-(5-methanesulfonyl-2-{[3-(4-{[(1R,4R)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}phenoxy)acetonitrile |
| 1108. | 2-(2-((3-(4-(((1S,4S)-4-(6-oxa-3-azabicyclo[3.1.1]heptan-3-yl)cyclohexyl)amino)-1- |
| | (2,2,2-trifluoroethyl)-1H-indol-2-yl)prop-2-yn-1-yl)amino)-5- |
| | (methylsulfonyl)phenoxy)acetonitrile |
| 1109. | 3-methoxy-N-methyl-4-{[3-(4-{[(1R,4R)-4-[2-oxa-8-azaspiro[4.5]decan-8- |
| | yl}cyclohexyl]amino}-1-(2,2,2-trifluoroethyl)-1H-indol-2-yl)prop-2-yn-1- |
| | yl]amino}benzamide |
| 1110. | 2-{3-[(4-methanesulfonyl-2-methoxyphenyl)amino]prop-1-yn-1-yl}-N-[(1R,4R)-4-{2- |
| | oxa-8-azaspiro[4.5]decan-8-yl}cyclohexyl]-1-(2,2,2-trifluoroethyl)-1H-indol-4-amine |
| 1111. | 3-methoxy-4-{[3-(4-{[(1R,4R)-4-{2-oxa-8-azaspiro[4.5]decan-8-yl}cyclohexyl]amino}- |
| | 1-(2,2,2-trifluoroethyl)-1H-indol-2-yl)prop-2-yn-1-yl]amino}benzene-1-sulfonamide |
| 1112. | 4-((3-(4-(((1R,4R)-4-(2-oxa-8-azaspiro[4.5]decan-8-yl)cyclohexyl)amino)-1-(2,2,2- |
| | trifluoroethyl)-1H-benzo[d]imidazol-2-yl)prop-2-yn-1-yl)amino)-3- |
| | methoxybenzenesulfonamide |
| 1113. | 4-((3-(4-(((1S,4S)-4-(2-oxa-8-azaspiro[4.5]decan-8-yl)cyclohexyl)amino)-1-(2,2,2- |
| | trifluoroethyl)-1H-benzo[d]imidazol-2-yl)prop-2-yn-1-yl)amino)-3- |
| | methoxybenzenesulfonamide |
| 1114. | 2-{3-[(4-methanesulfonyl-2-methoxyphenyl)amino]prop-1-yn-1-yl}-N-[(1R,4R)-4-{3- |
| | oxa-9-azaspiro[5.5]undecan-9-yl}cyclohexyl]-1-(2,2,2-trifluoroethyl)-1H-indol-4-amine |
| 1115. | N-((1S,4S)-4-(3-oxa-9-azaspiro[5.5]undecan-9-yl)cyclohexyl)-2-(3-((2-methoxy-4- |
| | (methylsulfonyl)phenyl)amino)prop-1-yn-1-yl)-1-(2,2,2-trifluoroethyl)-1H-indol-4-amine |
| 1116. | 3-methoxy-N-methyl-4-{[3-(4-{[(1R,4R)-4-{3-oxa-9-azaspiro[5.5]undecan-9- |
| | yl}cyclohexyl]amino}-1-(2,2,2-trifluoroethyl)-1H-indol-2-yl)prop-2-yn-1- |
| | yl]amino}benzamide |
| 1117. | 4-((3-(4-(((1S,4S)-4-(3-oxa-9-azaspiro[5.5]undecan-9-yl)cyclohexyl)amino)-1-(2,2,2- |
| | trifluoroethyl)-1H-indol-2-yl)prop-2-yn-1-yl)amino)-3-methoxy-N-methylbenzamide |
| 1118. | 3-methoxy-4-{[3-(4-{[(1R,4R)-4-{3-oxa-9-azaspiro[5.5]undecan-9- |
| | yl}cyclohexyl]amino}-1-(2,2,2-trifluoroethyl)-1H-indol-2-yl)prop-2-yn-1- |
| | yl]amino}benzene-1-sulfonamide |
| 1119. | 4-((3-(4-(((1S,4S)-4-(3-oxa-9-azaspiro[5.5]undecan-9-yl)cyclohexyl)amino)-1-(2,2,2- |
| | trifluoroethyl)-1H-indol-2-yl)prop-2-yn-1-yl)amino)-3-methoxybenzenesulfonamide |
| 1120. | 3-methoxy-N-methyl-4-{[3-(4-{[(1R,4R)-4-(pyrrolidin-1-yl)cyclohexyl]amino}-1-(2,2,2- |
| | trifluoroethyl)-1H-indol-2-yl)prop-2-yn-1-yl]amino}benzamide |
| 1121. | 3-methoxy-N-methyl-4-((3-(4-(((1S,4S)-4-(pyrrolidin-1-yl)cyclohexyl)amino)-1-(2,2,2- |
| | trifluoroethyl)-1H-indol-2-yl)prop-2-yn-1-yl)amino)benzamide |
| 1122. | 3-methoxy-4-{[3-(4-{[(1R,4R)-4-(pyrrolidin-1-yl)cyclohexyl]amino}-1-(2,2,2- |
| | trifluoroethyl)-1H-indol-2-yl)prop-2-yn-1-yl]amino}benzene-1-sulfonamide |
| 1123. | 3-methoxy-4-((3-(4-(((1S,4S)-4-(pyrrolidin-1-yl)cyclohexyl)amino)-1-(2,2,2- |
| | trifluoroethyl)-1H-indol-2-yl)prop-2-yn-1-yl)amino)benzenesulfonamide |
| 1124. | 2-{3-[(4-methanesulfonyl-2-methoxyphenyl)amino]prop-1-yn-1-yl}-N-[(1R,4R)-4- |
| | (pyrrolidin-1-yl)cyclohexyl]-1-(2,2,2-trifluoroethyl)-1H-indol-4-amine |
| 1125. | 2-(3-((2-methoxy-4-(methylsulfonyl)phenyl)amino)prop-1-yn-1-yl)-N-((1S,4S)-4- |
| | (pyrrolidin-1-yl)cyclohexyl)-1-(2,2,2-trifluoroethyl)-1H-indol-4-amine |
| 1126. | -{3-[(4-methanesulfonyl-2-methoxyphenyl)amino]prop-1-yn-1-yl}-N-[(1R,4R)-4-(3- |
| | methoxypyrrolidin-1-yl)cyclohexyl]-1-(2,2,2-trifluoroethyl)-1H-indol-4-amine |
| 1127. | 3-methoxy-4-{[3-(4-{[(1R,4R)-4-(3-methoxypyrrolidin-1-yl)cyclohexyl]amino}-1-(2,2,2- |
| | trifluoroethyl)-1H-indol-2-yl)prop-2-yn-1-yl]amino}benzene-1-sulfonamide |
| 1128. | 3-methoxy-N-methyl-4-{[3-(4-{[(1R,4R)-4-(3-methoxypyrrolidin-1- |
| | yl)cyclohexyl]amino}-1-(2,2,2-trifluoroethyl)-1H-indol-2-yl)prop-2-yn-1- |
| | yl]amino}benzamide |
| 1129. | 3-methoxy-4-((3-(4-(((1S,4S)-4-(3-methoxypyrrolidin-1-yl)cyclohexyl)amino)-1-(2,2,2- |
| | trifluoroethyl)-1H-indol-2-yl)prop-2-yn-1-yl)amino)-N-methylbenzamide |
| 1130. | 3-methoxy-4-{[3-(4-{[(1R,4R)-4-{hexahydro-1H-furo[3,4-c]pyrrol-5- |
| | yl}cyclohexyl]amino}-1-(2,2,2-trifluoroethyl)-1H-indol-2-yl)prop-2-yn-1- |
| | yl]amino}benzene-1-sulfonamide |
| 1131. | 3-methoxy-4-((3-(4-(((1S,4S)-4-(tetrahydro-1H-furo[3,4-c]pyrrol-5(3H)- |
| | yl)cyclohexyl)amino)-1-(2,2,2-trifluoroethyl)-1H-indol-2-yl)prop-2-yn-1- |
| | yl)amino)benzenesulfonamide |
| 1132. | 2-{3-[(4-methanesulfonyl-2-methoxyphenyl)amino]prop-1-yn-1-yl}-N-[(1R,4R)-4- |
| | {hexahydro-1H-furo[3,4-c]pyrrol-5-yl}cyclohexyl]-1-(2,2,2-trifluoroethyl)-1H-indol-4- |
| | amine |
| 1133. | 2-(3-((2-methoxy-4-(methylsulfonyl)phenyl)amino)prop-1-yn-1-yl)-N-((1R,4R)-4- |
| | (tetrahydro-1H-furo[3,4-c]pyrrol-5(3H)-yl)cyclohexyl)-1-(2,2,2-trifluoroethyl)-1H-indol- |
| | 4-amine |
| 1134. | 3-methoxy-N-methyl-4-{[3-(4-{[(1R,4R)-4-{hexahydro-1H-furo[3,4-c]pyrrol-5- |
| | yl}cyclohexyl]amino}-1-(2,2,2-trifluoroethyl)-1H-indol-2-yl)prop-2-yn-1- |
| | yl]amino}benzamide |
| 1135. | 3-methoxy-N-methyl-4-((3-(4-(((1S,4S)-4-(tetrahydro-1H-furo[3,4-c]pyrrol-5(3H)- |
| | yl)cyclohexyl)amino)-1-(2,2,2-trifluoroethyl)-1H-indol-2-yl)prop-2-yn-1- |
| | yl)amino)benzamide |
| 1136. | 2-{3-[(4-methanesulfonyl-2-methoxyphenyl)amino]prop-1-yn-1-yl}-N-[(1R,4R)-4-[4- |
| | (trifluoromethyl)piperidin-1-yl]cyclohexyl]-1-(2,2,2-trifluoroethyl)-1H-indol-4-amine |
| 1137. | 2-(3-((2-methoxy-4-(methylsulfonyl)phenyl)amino)prop-1-yn-1-yl)-1-(2,2,2- |
| | trifluoroethyl)-N-((1S,4S)-4-(4-(trifluoromethyl)piperidin-1-yl)cyclohexyl)-1H-indol-4- |
| | amine |
| 1138. | 3-methoxy-N-methyl-4-{[3-(4-{[(1R,4R)-4-[4-(trifluoromethyl)piperidin-1- |
| | yl]cyclohexyl]amino}-1-(2,2,2-trifluoroethyl)-1H-indol-2-yl)prop-2-yn-1- |
| | yl]amino}benzamide |
| 1139. | 3-methoxy-N-methyl-4-((3-(1-(2,2,2-trifluoroethyl)-4-(((1S,4S)-4-(4- |
| | (trifluoromethyl)piperidin-1-yl)cyclohexyl)amino)-1H-indol-2-yl)prop-2-yn-1- |
| | yl)amino)benzamide |
| 1140. | 3-methoxy-4-{[3-(4-{[(1R,4R)-4-[4-(trifluoromethyl)piperidin-1-yl]cyclohexyl]amino}- |
| | 1-(2,2,2-trifluoroethyl)-1H-indol-2-yl)prop-2-yn-1-yl]amino}benzene-1-sulfonamide |
| 1141. | 3-methoxy-4-((3-(1-(2,2,2-trifluoroethyl)-4-(((1S,4S)-4-(4-(trifluoromethyl)piperidin-1- |
| | yl)cyclohexyl)amino)-1H-indol-2-yl)prop-2-yn-1-yl)amino)benzenesulfonamide |
| 1142. | 2-{3-[(4-methanesulfonyl-2-methoxyphenyl)amino]prop-1-yn-1-yl}-N-[(1R,4R)-4-(4- |
| | methanesulfonylpiperidin-1-yl)cyclohexyl]-1-(2,2,2-trifluoroethyl)-1H-indol-4-amine |
| 1143. | 2-(3-((2-methoxy-4-(methylsulfonyl)phenyl)amino)prop-1-yn-1-yl)-N-((1S,4S)-4-(4- |
| | (methylsulfonyl)piperidin-1-yl)cyclohexyl)-1-(2,2,2-trifluoroethyl)-1H-indol-4-amine |
| 1144. | 3-methoxy-N-methyl-4-{[3-(4-{[(1R,4R)-4-(4-methanesulfonylpiperidin-1- |
| | yl)cyclohexyl]amino}-1-(2,2,2-trifluoroethyl)-1H-indol-2-yl)prop-2-yn-1- |
| | yl]amino}benzamide |
| 1145. | 3-methoxy-N-methyl-4-((3-(4-(((1S,4S)-4-(4-(methylsulfonyl)piperidin-1- |
| | yl)cyclohexyl)amino)-1-(2,2,2-trifluoroethyl)-1H-indol-2-yl)prop-2-yn-1- |
| | yl)amino)benzamide |
| 1146. | 3-methoxy-4-{[3-(4-{[(1R,4R)-4-(4-methanesulfonylpiperidin-1-yl)cyclohexyl]amino}-1- |
| | (2,2,2-trifluoroethyl)-1H-indol-2-yl)prop-2-yn-1-yl]amino}benzene-1-sulfonamide |
| 1147. | 3-methoxy-4-((3-(4-(((1S,4S)-4-(4-(methylsulfonyl)piperidin-1-yl)cyclohexyl)amino)-1- |
| | (2,2,2-trifluoroethyl)-1H-indol-2-yl)prop-2-yn-1-yl)amino)benzenesulfonamide |
| 1148. | 1-[(1S,3R)-3-[(2-{3-[(4-methanesulfonyl-2-methoxyphenyl)amino]prop-1-yn-1-yl}-1- |
| | (2,2,2-trifluoroethyl)-1H-indol-4-yl)amino]cyclohexyl]-4,5-dihydro-1H-1,2,3,4-tetrazol- |
| | 5-one |
| 1149. | 2-(3-((2-methoxy-4-(methylsulfonyl)phenyl)amino)prop-1-yn-1-yl)-N-((1R,4R)-4- |
| | morpholinocyclohexyl)-1-(oxiran-2-ylmethyl)-1H-indol-4-amine |
| 1150. | 2-((2-(3-((2-methoxy-4-(methylsulfonyl)phenyl)amino)prop-1-yn-1-yl)-4-(((1R,4R)-4- |
| | morpholinocyclohexyl)amino)-1H-indol-1-yl)methyl)acrylonitrile |
| 1151. | N-((1R,4R)-4-(2-oxa-8-azaspiro[4.5]decan-8-yl)cyclohexyl)-2-(3-((2-methoxy-4- |
| | (methylsulfonyl)phenyl)amino)prop-1-yn-1-yl)-1-(2,2,2-trifluoroethyl)-1H- |
| | benzo[d]imidazol-4-amine |
| 1152. | N-((1S,4S)-4-(2-oxa-8-azaspiro[4.5]decan-8-yl)cyclohexyl)-2-(3-((2-methoxy-4- |
| | (methylsulfonyl)phenyl)amino)prop-1-yn-1-yl)-1-(2,2,2-trifluoroethyl)-1H- |
| | benzo[d]imidazol-4-amine |
| 1153. | 4-((3-(4-(((1R,4R)-4-(2-oxa-8-azaspiro[4.5]decan-8-yl)cyclohexyl)amino)-1-(2,2,2- |
| | trifluoroethyl)-1H-benzo[d]imidazol-2-yl)prop-2-yn-1-yl)amino)-3-methoxy-N- |
| | methylbenzamide |
| 1154. | 4-((3-(4-(((1S,4S)-4-(2-oxa-8-azaspiro[4.5]decan-8-yl)cyclohexyl)amino)-1-(2,2,2- |
| | trifluoroethyl)-1H-benzo[d]imidazol-2-yl)prop-2-yn-1-yl)amino)-3-methoxy-N- |
| | methylbenzamide |
| 1155. | 1-[1-(2-hydroxyethyl)piperidin-4-yl]-3-(2-{3-[(4-methanesulfonyl-2- |
| | methoxyphenyl)amino]prop-1-yn-1-yl}-1-(2,2,2-trifluoroethyl)-1H-indol-4-yl)urea |
| 1156. | 3-methoxy-4-({3-[4-({2-oxaspiro[3.3]heptan-6-yl}amino)-1-(2,2,2-trifluoroethyl)-1H- |
| | indol-2-yl]prop-2-yn-1-yl}amino)benzene-1-sulfonamide |
| 1157. | [1-(chloromethyl)-3-[(2-{3-[(4-methanesulfonyl-2-methoxyphenyl)amino]prop-1-yn-1- |
| | yl}-1-(2,2,2-trifluoroethyl)-1H-indol-4-yl)amino]cyclobutyl]methanol |
| 1158. | 2-{3-[(4-methanesulfonyl-2-methoxyphenyl)amino]prop-1-yn-1-yl}-N-{2- |
| | oxaspiro[3.3]heptan-6-yl}-1-(2,2,2-trifluoroethyl)-1H-indol-4-amine |
| 1159. | 4-({3-[4-({2-azaspiro[3.3]heptan-6-yl}amino)-1-(2,2,2-trifluoroethyl)-1H-indol-2- |
| | yl]prop-2-yn-1-yl}amino)-3-methoxybenzene-1-sulfonamide |
| 1160. | N-{2-azaspiro[3.3]heptan-6-yl}-2-{3-[(4-methanesulfonyl-2-methoxyphenyl)amino]prop- |
| | 1-yn-1-yl}-1-(2,2,2-trifluoroethyl)-1H-indol-4-amine |
| 1161. | 3-methoxy-N-methyl-4-{[3-(4-{[(1R,4R)-4-(methylamino)cyclohexyl]amino}-1-(2,2,2- |
| | trifluoroethyl)-1H-indol-2-yl)prop-2-yn-1-yl]amino}benzamide |
| 1162. | 3-methoxy-N-methyl-4-{[3-(4-{[(1R,4R)-4-aminocyclohexyl]amino}-1-(2,2,2- |
| | trifluoroethyl)-1H-indol-2-yl)prop-2-yn-1-yl]amino}benzamide |
| 1163. | 3-methoxy-N-methyl-4-{[3-(4-{[(1S,4S)-4-aminocyclohexyl]amino}-1-(2,2,2- |
| | trifluoroethyl)-1H-indol-2-yl)prop-2-yn-1-yl]amino}benzamide |
| 1164. | re1-(1R,3R)-N¹-(2-{3-[(4-methanesulfonyl-2-methoxyphenyl)amino]prop-1-yn-1-yl}-1- |
| | (2,2,2-trifluoroethyl)-1H-indol-4-yl)cyclohexane-1,3-diamine |
| 1165. | rac-(1R,3S)-N¹-(2-{3-[(4-methanesulfonyl-2-methoxyphenyl)amino]prop-1-yn-1-yl}-1- |
| | (2,2,2-trifluoroethyl)-1H-indol-4-yl)cyclohexane-1,3-diamine |
| 1166. | (1R,2S)-N¹-(2-{3-[(4-methanesulfonyl-2-methoxyphenyl)amino]prop-1-yn-1-yl}-1- |
| | (2,2,2-trifluoroethyl)-1H-indol-4-yl)cyclohexane-1,2-diamine |
| 1167. | rac-(1R,2S)-N¹-(2-{3-[(4-methanesulfonyl-2-methoxyphenyl)amino]prop-1-yn-1-yl}-1- |
| | (2,2,2-trifluoroethyl)-1H-indol-4-yl)cyclohexane-1,2-diamine |
| 1168. | rel-(1R,3S)-3-[(2-{3-[(4-methanesulfonyl-2-methoxyphenyl)amino]prop-1-yn-1-yl}-1- |
| | (2,2,2-trifluoroethyl)-1H-indol-4-yl)amino]cyclohexan-1-ol |
| 1169. | 3-methoxy-N-methyl-4-{[3-(4-{[(1R,4R)-4-hydroxy-4-methylcyclohexyl]amino}-1- |
| | (2,2,2-trifluoroethyl)-1H-indol-2-yl)prop-2-yn-1-yl]amino}benzamide |
| 1170. | 3-methoxy-N-methyl-4-{[3-(4-{[(1S,4S)-4-hydroxy-4-methylcyclohexyl]amino}-1- |
| | (2,2,2-trifluoroethyl)-1H-indol-2-yl)prop-2-yn-1-yl]amino}benzamide |
| 1171. | 4-[(3-{4-[(4-cyanocyclohexyl)amino]-1-(2,2,2-trifluoroethyl)-1H-indol-2-yl}prop-2-yn-1- |
| | yl)amino]-3-methoxy-N-methylbenzamide |
| 1172. | 3-methoxy-N-methyl-4-{[3-(4-{[(1R,4R)-4-cyanocyclohexyl]amino}-1-(2,2,2- |
| | trifluoroethyl)-1H-indol-2-yl)prop-2-yn-1-yl]amino}benzamide |
| 1173. | 3-methoxy-N-methyl-4-{[3-(4-{[(1S,4S)-4-cyanocyclohexyl]amino}-1-(2,2,2- |
| | trifluoroethyl)-1H-indol-2-yl)prop-2-yn-1-yl]amino}benzamide |
| 1174. | 3-[(2-{3-[(4-methanesulfonyl-2-methoxyphenyl)amino]prop-1-yn-1-yl}-1-(2,2,2- |
| | trifluoroethyl)-1H-indol-4-yl)amino]cyclohexane-1-carboxylic acid |
| 1175. | 2-fluoro-N¹-(2-(3-((2-methoxy-4-(methylsulfonyl)phenyl)amino)prop-1-yn-1-yl)-1-(2,2,2- |
| | trifluoroethyl)-1H-indol-4-yl)cyclohexane-1,4-diamine |
| 1176. | (1R,2R,4S)-2-fluoro-N¹-(2-(3-((2-methoxy-4-(methylsulfonyl)phenyl)amino)prop-1-yn-1- |
| | yl)-1-(2,2,2-trifluoroethyl)-1H-indol-4-yl)cyclohexane-1,4-diamine |
| 1177. | 2-fluoro-N¹-(2-{3-[(4-methanesulfonyl-2-methoxyphenyl)amino]prop-1-yn-1-yl}-1- |
| | (2,2,2-trifluoroethyl)-1H-indol-4-yl)-N⁴-methylcyclohexane-1,4-diamine |
| 1178. | (1R,2R,4S)-2-fluoro-N¹-(2-(3-((2-methoxy-4-(methylsulfonyl)phenyl)amino)prop-1-yn-1- |
| | yl)-1-(2,2,2-trifluoroethyl)-1H-indol-4-yl)-N⁴-methylcyclohexane-1,4-diamine |
| 1179. | 2-fluoro-N¹-(2-(3-((2-methoxy-4-(methylsulfonyl)phenyl)amino)prop-1-yn-1-yl)-1-(2,2,2- |
| | trifluoroethyl)-1H-indol-4-yl)-N⁴,N⁴-dimethylcyclohexane-1,4-diamine |
| 1180. | (1R,2R,4S)-2-fluoro-N¹-(2-(3-((2-methoxy-4-(methylsulfonyl)phenyl)amino)prop-1-yn-1- |
| | yl)-1-(2,2,2-trifluoroethyl)-1H-indol-4-yl)-N⁴,N⁴-dimethylcyclohexane-1,4-diamine |
| 1181. | (3S,4R)-3-fluoro-4-[(2-{3-[(4-methanesulfonyl-2-methoxyphenyl)amino]prop-1-yn-1-yl}- |
| | 1-(2,2,2-trifluoroethyl)-1H-indol-4-yl)amino]cyclohexan-1-ol |
| 1182. | 3-methoxy-4-{[3-(4-{[(1R,4R)-4-[bis(2-hydroxyethyl)amino]cyclohexyl]amino}-1- |
| | (2,2,2-trifluoroethyl)-1H-indol-2-yl)prop-2-yn-1-yl]amino}benzene-1-sulfonamide |
| 1183. | 3-methoxy-4-{[3-(4-{[(1S,4S)-4-[bis(2-hydroxyethyl)amino]cyclohexyl]amino}-1-(2,2,2- |
| | trifluoroethyl)-1H-indol-2-yl)prop-2-yn-1-yl]amino}benzene-1-sulfonamide |
| 1184. | 4-((3-(4-(((1R,4R)-4-(dimethylamino)cyclohexyl)amino)-1-(2,2,2-trifluoroethyl)-1H- |
| | indol-2-yl)prop-2-yn-1-yl)amino)-3-methoxybenzenesulfonamide |
| 1185. | 4-((3-(4-(((1S,4S)-4-(dimethylamino)cyclohexyl)amino)-1-(2,2,2-trifluoroethyl)-1H- |
| | indol-2-yl)prop-2-yn-1-yl)amino)-3-methoxybenzenesulfonamide |
| 1186. | 3-methoxy-N-(1,2-oxazol-3-yl)-4-{[3-(4-{[(1R,4R)-4- |
| | (dimethylamino)cyclohexyl]amino}-1-(2,2,2-trifluoroethyl)-1H-indol-2-yl)prop-2-yn-1- |
| | yl]amino}benzene-1-sulfonamide |
| 1187. | 3-methoxy-N-(oxan-4-yl)-4-{[3-(4-{[(1R,4R)-4-(dimethylamino)cyclohexyl]amino}-1- |
| | (2,2,2-trifluoroethyl)-1H-indol-2-yl)prop-2-yn-1-yl]amino}benzene-1-sulfonamide |
| 1188. | 3-methoxy-N-(1,2-oxazol-3-yl)-4-{[3-(4-{[(1S,4S)-4- |
| | (dimethylamino)cyclohexyl]amino}-1-(2,2,2-trifluoroethyl)-1H-indol-2-yl)prop-2-yn-1- |
| | yl]amino}benzene-1-sulfonamide |
| 1189. | 3-methoxy-N-(oxan-4-yl)-4-{[3-(4-{[(1S,4S)-4-(dimethylamino)cyclohexyl]amino}-1- |
| | (2,2,2-trifluoroethyl)-1H-indol-2-yl)prop-2-yn-1-yl]amino}benzene-1-sulfonamide |
| 1190. | N-(3-(4-(((1S,4S)-4-(dimethylamino)cyclohexyl)amino)-1-(2,2,2-trifluoroethyl)-1H- |
| | indol-2-yl)prop-2-yn-1-yl)-N-(2-hydroxy-4-(methylsulfonyl)phenyl)propionamide |
| 1191. | N-(3-(4-(((1R,4R)-4-(dimethylamino)cyclohexyl)amino)-1-(2,2,2-trifluoroethyl)-1H- |
| | indol-2-yl)prop-2-yn-1-yl)-N-(2-hydroxy-4-(methylsulfonyl)phenyl)propionamide |
| 1192. | 3-methoxy-N-(5-methyl-1,2-oxazol-3-yl)-4-{[3-(4-{[(1S,4S)-4- |
| | (dimethylamino)cyclohexyl]amino}-1-(2,2,2-trifluoroethyl)-1H-indol-2-yl)prop-2-yn-1- |
| | yl]amino}benzene-1-sulfonamide |
| 1193. | 3-methoxy-N-(5-methyl-1,2-oxazol-3-yl)-4-{[3-(4-{[(1R,4R)-4- |
| | (dimethylamino)cyclohexyl]amino}-1-(2,2,2-trifluoroethyl)-1H-indol-2-yl)prop-2-yn-1- |
| | yl]amino}benzene-1-sulfonamide |
| 1194. | (1S,4S)-N⁴-(2-{3-[(4-methanesulfonyl-2-methoxyphenyl)amino]prop-1-yn-1-yl}-1- |
| | (2,2,2-trifluoroethyl)-1H-indol-4-yl)-N¹-(2-methoxyethyl)-N¹-methylcyclohexane-1,4- |
| | diamine |
| 1195. | (1R,4R)-N⁴-(2-{3-[(4-methanesulfonyl-2-methoxyphenyl)amino]prop-1-yn-1-yl}-1- |
| | (2,2,2-trifluoroethyl)-1H-indol-4-yl)-N¹-(2-methoxyethyl)-N¹-methylcyclohexane-1,4- |
| | diamine |
| 1196. | 3-methoxy-4-{[3-(4-{[(1S,4S)-4-[(3S,4S)-3,4-dihydroxypyrrolidin-1- |
| | yl]cyclohexyl]amino}-1-(2,2,2-trifluoroethyl)-1H-indol-2-yl)prop-2-yn-1- |
| | yl]amino}benzene-1-sulfonamide |
| 1197. | 4-((3-(4-(((1R,4R)-4-((3R,4R)-3,4-dihydroxypyrrolidin-1-yl)cyclohexyl)amino)-1-(2,2,2- |
| | trifluoroethyl)-1H-indol-2-yl)prop-2-yn-1-yl)amino)-3-methoxybenzenesulfonamide |
| 1198. | 4-((3-(4-(((1R,4S)-4-((3S,4S)-3,4-dihydroxypyrrolidin-1-yl)cyclohexyl)amino)-1-(2,2,2- |
| | trifluoroethyl)-1H-indol-2-yl)prop-2-yn-1-yl)amino)-3-methoxybenzenesulfonamide |
| 1199. | 3-methoxy-4-{[3-(4-{[(1R,4R)-4-[bis(2-methoxyethyl)amino]cyclohexyl]amino}-1- |
| | (2,2,2-trifluoroethyl)-1H-indol-2-yl)prop-2-yn-1-yl]amino}benzene-1-sulfonamide |
| 1200. | 3-methoxy-4-{[3-(4-{[(1S,4S)-4-[bis(2-methoxyethyl)amino]cyclohexyl]amino}-1-(2,2,2- |
| | trifluoroethyl)-1H-indol-2-yl)prop-2-yn-1-yl]amino}benzene-1-sulfonamide |
| 1201. | 4-({3-[4-({1,4-dioxaspiro[4.5]decan-8-yl}amino)-1-(2,2,2-trifluoroethyl)-1H-indol-2- |
| | yl]prop-2-yn-1-yl}amino)-3-methoxybenzene-1-sulfonamide |
| 1202. | N-{1,4-dioxaspiro[4.5]decan-8-yl}-2-{3-[(4-methanesulfonyl-2- |
| | methoxyphenyl)amino]prop-1-yn-1-yl}-1-(2,2,2-trifluoroethyl)-1H-indol-4-amine |
| 1203. | 3-methoxy-4-[(3-{4-[(4-oxocyclohexyl)amino]-1-(2,2,2-trifluoroethyl)-1H-indol-2- |
| | yl}prop-2-yn-1-yl)amino]benzene-1-sulfonamide |
| 1204. | 4-[(2-{3-[(4-methanesulfonyl-2-methoxyphenyl)amino]prop-1-yn-1-yl}-1-(2,2,2- |
| | trifluoroethyl)-1H-indol-4-yl)amino]cyclohexan-1-one |
| 1205. | (1R,4R)-N⁴-[2-(3-{[2-methoxy-4-(morpholine-4-sulfonyl)phenyl]amino}prop-1-yn-1- |
| | yl)-1-(2,2,2-trifluoroethyl)-1H-indol-4-yl]-N¹,N¹-dimethylcyclohexane-1,4-diamine |
| 1206. | (1S,4S)-N⁴-[2-(3-{[2-methoxy-4-(morpholine-4-sulfonyl)phenyl]amino}prop-1-yn-1-yl)- |
| | 1-(2,2,2-trifluoroethyl)-1H-indol-4-yl]-N¹,N¹-dimethylcyclohexane-1,4-diamine |
| 1207. | N,N-bis(2-hydroxyethyl)-3-methoxy-4-{[3-(4-{[(1S,4S)-4- |
| | (dimethylamino)cyclohexyl]amino}-1-(2,2,2-trifluoroethyl)-1H-indol-2-yl)prop-2-yn-1- |
| | yl]amino}benzene-1-sulfonamide |
| 1208. | N,N-bis(2-hydroxyethyl)-3-methoxy-4-{[3-(4-{[(1R,4R)-4- |
| | (dimethylamino)cyclohexyl]amino}-1-(2,2,2-trifluoroethyl)-1H-indol-2-yl)prop-2-yn-1- |
| | yl]amino}benzene-1-sulfonamide |
| 1209. | 3-methoxy-N-methyl-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzene-1-sulfonamide |
| 1210. | 3-methoxy-N-methyl-4-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}benzene-1-sulfonamide |
| 1211. | 3-methoxy-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzamide |
| 1212. | 3-methoxy-N-methyl-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzamide |
| 1213. | 3-methoxy-N-methyl-4-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}benzamide |
| 1214. | (1R,4R)-N⁴-{2-[3-({2-methoxy-4-[(4-methylpiperazin-1-yl)sulfonyl]phenyl}amino)prop- |
| | 1-yn-1-yl]-1-(2,2,2-trifluoroethyl)-1H-indol-4-yl}-N¹,N¹-dimethylcyclohexane-1,4- |
| | diamine |
| 1215. | (1R,4R)-N⁴-(2-{3-[(4-methanesulfonyl-2-methoxyphenyl)amino]prop-1-yn-1-yl}-1- |
| | [(oxiran-2-yl)methyl]-1H-indol-4-yl)-N¹,N¹-dimethylcyclohexane-1,4-diamine |
| 1216. | 2-[(2-{3-[(4-methanesulfonyl-2-methoxyphenyl)amino]prop-1-yn-1-yl}-4-{[(1R,4R)-4- |
| | (dimethylamino)cyclohexyl]amino}-1H-indol-1-yl)methyl]prop-2-enenitrile |
| 1217. | 5-methanesulfonyl-2-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}phenol |
| 1218. | 5-methanesulfonyl-2-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}phenol |
| 1219. | 2-(5-methanesulfonyl-2-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}phenoxy)acetonitrile |
| 1220. | 2-(5-methanesulfonyl-2-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}phenoxy)acetonitrile |
| 1221. | N-(3-methoxy-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzenesulfonyl)acetamide |
| 1222. | N-(3-methoxy-4-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}benzenesulfonyl)acetamide |
| 1223. | N-(3-methoxy-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzenesulfonyl)propanamide |
| 1224. | N-(3-methoxy-4-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}benzenesulfonyl)propanamide |
| 1225. | 2-(5-methanesulfonyl-2-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}phenoxy)ethan-1-ol |
| 1226. | 2-(5-methanesulfonyl-2-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}phenoxy)ethan-1-ol |
| 1227. | (1R,4R)-N⁴-[2-(3-{[4-methanesulfonyl-2-(2-methoxyethoxy)phenyl]amino}prop-1-yn-1- |
| | yl)-1-(2,2,2-trifluoroethyl)-1H-indol-4-yl]-N¹,N¹-dimethylcyclohexane-1,4-diamine |
| 1228. | N-(3-(4-(((1R,4R)-4-(dimethylamino)cyclohexyl)amino)-1-(2,2,2-trifluoroethyl)-1H- |
| | indol-2-yl)prop-2-yn-1-yl)-N-(2-hydroxy-4-(methylsulfonyl)phenyl)isobutyramide |
| 1229. | N-(3-(4-(((1S,4S)-4-(dimethylamino)cyclohexyl)amino)-1-(2,2,2-trifluoroethyl)-1H- |
| | indol-2-yl)prop-2-yn-1-yl)-N-(2-hydroxy-4-(methylsulfonyl)phenyl)isobutyramide |
| 1230. | (1R,4R)-N⁴-[2-(3-{[4-methanesulfonyl-2-(2,2,2-trifluoroethoxy)phenyl]amino}prop-1- |
| | yn-1-yl)-1-(2,2,2-trifluoroethyl)-1H-indol-4-yl]-N¹,N¹-dimethylcyclohexane-1,4-diamine |
| 1231. | (1S,4S)-N⁴-[2-(3-{[4-methanesulfonyl-2-(2,2,2-trifluoroethoxy)phenyl]amino}prop-1- |
| | yn-1-yl)-1-(2,2,2-trifluoroethyl)-1H-indol-4-yl]-N¹,N¹-dimethylcyclohexane-1,4-diamine |
| 1232. | (1S,4S)-N⁴-{2-[3-(2-amino-4-methanesulfonylphenoxy)prop-1-yn-1-yl]-1-(2,2,2- |
| | trifluoroethyl)-1H-indol-4-yl}-N¹,N¹-dimethylcyclohexane-1,4-diamine |
| 1233. | (1R,4R)-N⁴-(2-{3-[(2-ethoxy-4-methanesulfonylphenyl)amino]prop-1-yn-1-yl}-1-(2,2,2- |
| | trifluoroethyl)-1H-indol-4-yl)-N¹,N¹-dimethylcyclohexane-1,4-diamine |
| 1234. | (1S,4S)-N⁴-(2-{3-[(2-ethoxy-4-methanesulfonylphenyl)amino]prop-1-yn-1-yl}-1-(2,2,2- |
| | trifluoroethyl)-1H-indol-4-yl)-N¹,N¹-dimethylcyclohexane-1,4-diamine |
| 1235. | 3-hydroxy-N-methyl-4-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}benzamide |
| 1236. | 3-hydroxy-N-methyl-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzamide |
| 1237. | 3-(2-methoxyethoxy)-N-methyl-4-{[3-(4-{[(1S,4S)-4- |
| | (dimethylamino)cyclohexyl]amino}-1-(2,2,2-trifluoroethyl)-1H-indol-2-yl)prop-2-yn-1- |
| | yl]amino}benzamide |
| 1238. | (1R,4R)-N⁴-{2-[3-(2-amino-4-methanesulfonylphenoxy)prop-1-yn-1-yl]-1-(2,2,2- |
| | trifluoroethyl)-1H-indol-4-yl}-N¹,N¹-dimethylcyclohexane-1,4-diamine |
| 1239. | N-ethyl-3-methoxy-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzamide |
| 1240. | N-ethyl-3-methoxy-4-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}benzamide |
| 1241. | 3-(2-methoxyethoxy)-N-methyl-4-{[3-(4-{[(1R,4R)-4- |
| | (dimethylamino)cyclohexyl]amino}-1-(2,2,2-trifluoroethyl)-1H-indol-2-yl)prop-2-yn-1- |
| | yl]amino}benzamide |
| 1242. | 3-(2-fluoroethoxy)-N-methyl-4-{[3-(4-{[(1R,4R)-4-(dimethylamino)cyclohexyl]amino}- |
| | 1-(2,2,2-trifluoroethyl)-1H-indol-2-yl)prop-2-yn-1-yl]amino}benzamide |
| 1243. | 3-(2-fluoroethoxy)-N-methyl-4-{[3-(4-{[(1S,4S)-4-(dimethylamino)cyclohexyl]amino}- |
| | 1-(2,2,2-trifluoroethyl)-1H-indol-2-yl)prop-2-yn-1-yl]amino}benzamide |
| 1244. | 3-(fluoromethoxy)-N-methyl-4-{[3-(4-{[(1R,4R)-4-(dimethylamino)cyclohexyl]amino}- |
| | 1-(2,2,2-trifluoroethyl)-1H-indol-2-yl)prop-2-yn-1-yl]amino}benzamide |
| 1245. | 3-(fluoromethoxy)-N-methyl-4-{[3-(4-{[(1S,4S)-4-(dimethylamino)cyclohexyl]amino}-1- |
| | (2,2,2-trifluoroethyl)-1H-indol-2-yl)prop-2-yn-1-yl]amino}benzamide |
| 1246. | (1S,4S)-N⁴-(2-{3-[4-methanesulfonyl-2-(methylamino)phenoxy]prop-1-yn-1-yl}-1- |
| | (2,2,2-trifluoroethyl)-1H-indol-4-yl)-N¹,N¹-dimethylcyclohexane-1,4-diamine |
| 1247. | (1R,4R)-N⁴-(2-{3-[4-methanesulfonyl-2-(methylamino)phenoxy]prop-1-yn-1-yl}-1- |
| | (2,2,2-trifluoroethyl)-1H-indol-4-yl)-N¹,N¹-dimethylcyclohexane-1,4-diamine |
| 1248. | 3-methoxy-N-methyl-4-{[3-(4-{[(1S,4S)-4-(methylamino)cyclohexyl]amino}-1-(2,2,2- |
| | trifluoroethyl)-1H-indol-2-yl)prop-2-yn-1-yl]amino}benzamide |
| 1249. | (1S,4S)-N⁴-(2-{3-[2-(dimethylamino)-4-methanesulfonylphenoxy]prop-1-yn-1-yl}-1- |
| | (2,2,2-trifluoroethyl)-1H-indol-4-yl)-N¹,N¹-dimethylcyclohexane-1,4-diamine |
| 1250. | (1R,4R)-N⁴-(2-{3-[2-(dimethylamino)-4-methanesulfonylphenoxy]prop-1-yn-1-yl}-1- |
| | (2,2,2-trifluoroethyl)-1H-indol-4-yl)-N¹,N¹-dimethylcyclohexane-1,4-diamine |
| 1251. | 3-(cyanomethoxy)-N-methyl-4-{[3-(4-{[(1R,4R)-4-(dimethylamino)cyclohexyl]amino}- |
| | 1-(2,2,2-trifluoroethyl)-1H-indol-2-yl)prop-2-yn-1-yl]amino}benzamide |
| 1252. | 3-(2-cyanoethoxy)-N-methyl-4-{[3-(4-{[(1R,4R)-4-(dimethylamino)cyclohexyl]amino}- |
| | 1-(2,2,2-trifluoroethyl)-1H-indol-2-yl)prop-2-yn-1-yl]amino}benzamide |
| 1253. | N-(5-methanesulfonyl-2-{[3-(4-{[(1R,4R)-4-(dimethylamino)cyclohexyl]amino}-1- |
| | (2,2,2-trifluoroethyl)-1H-indol-2-yl)prop-2-yn-1-yl]oxy}phenyl)acetamide |
| 1254. | N-(2-((3-(4-(((1S,4S)-4-(dimethylamino)cyclohexyl)amino)-1-(2,2,2-trifluoroethyl)-1H- |
| | indol-2-yl)prop-2-yn-1-yl)oxy)-5-(methylsulfonyl)phenyl)acetamide |
| 1255. | (1R,4R)-N¹-(2-(3-((2-(fluoromethoxy)-4-(methylsulfonyl)phenyl)amino)prop-1-yn-1-yl)- |
| | 1-(2,2,2-trifluoroethyl)-1H-indol-4-yl)-N⁴,N⁴-dimethylcyclohexane-1,4-diamine |
| 1256. | (1S,4S)-N¹-(2-(3-((2-(fluoromethoxy)-4-(methylsulfonyl)phenyl)amino)prop-1-yn-1-yl)- |
| | 1-(2,2,2-trifluoroethyl)-1H-indol-4-yl)-N⁴,N⁴-dimethylcyclohexane-1,4-diamine |
| 1257. | (1R,4R)-N⁴-[2-(3-{[4-(ethanesulfonyl)-2-methoxyphenyl]amino}prop-1-yn-1-yl)-1- |
| | (2,2,2-trifluoroethyl)-1H-indol-4-yl]-N¹,N¹-dimethylcyclohexane-1,4-diamine |
| 1258. | (1S,4S)-N⁴-[2-(3-{[4-(ethanesulfonyl)-2-methoxyphenyl]amino}prop-1-yn-1-yl)-1-(2,2,2- |
| | trifluoroethyl)-1H-indol-4-yl]-N¹,N¹-dimethylcyclohexane-1,4-diamine |
| 1259. | 2-(3-methoxy-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}phenyl)-2-methylpropanenitrile |
| 1260. | 2-(4-((3-(4-(((1S,4S)-4-(dimethylamino)cyclohexyl)amino)-1-(2,2,2-trifluoroethyl)-1H- |
| | indol-2-yl)prop-2-yn-1-yl)amino)-3-methoxyphenyl)-2-methylpropanenitrile |
| 1261. | 3-(cyanomethoxy)-4-{[3-(4-{[(1R,4R)-4-(dimethylamino)cyclohexyl]amino}-1-(2,2,2- |
| | trifluoroethyl)-1H-indol-2-yl)prop-2-yn-1-yl]amino}benzene-1-sulfonamide |
| 1262. | 3-(cyanomethoxy)-4-((3-(4-(((1S,4S)-4-(dimethylamino)cyclohexyl)amino)-1-(2,2,2- |
| | trifluoroethyl)-1H-indol-2-yl)prop-2-yn-1-yl)amino)benzenesulfonamide |
| 1263. | 3-(3-(4-(((1R,4R)-4-(dimethylamino)cyclohexyl)amino)-1-(2,2,2-trifluoroethyl)-1H- |
| | indol-2-yl)prop-2-yn-1-yl)-6-(methylsulfonyl)benzo[d]oxazol-2(3H)-one |
| 1264. | 3-(3-(4-(((1S,4S)-4-(dimethylamino)cyclohexyl)amino)-1-(2,2,2-trifluoroethyl)-1H-indol- |
| | 2-yl)prop-2-yn-1-yl)-6-(methylsulfonyl)benzo[d]oxazol-2(3H)-one |
| 1265. | 3-methoxy-N,N-dimethyl-4-{[3-(4-{[(1R,4R)-4-(dimethylamino)cyclohexyl]amino}-1- |
| | (2,2,2-trifluoroethyl)-1H-indol-2-yl)prop-2-yn-1-yl]amino}benzene-1-sulfonamide |
| 1266. | (1R,4R)-N¹-(2-(3-(((3S,4R)-3-methoxytetrahydro-2H-pyran-4-yl)amino)prop-1-yn-1-yl)- |
| | 1-(2,2,2-trifluoroethyl)-1H-indol-4-yl)-N⁴,N⁴-dimethylcyclohexane-1,4-diamine |
| 1267. | (1S,4S)-N¹-(2-(3-(((3S,4R)-3-methoxytetrahydro-2H-pyran-4-yl)amino)prop-1-yn-1-yl)- |
| | 1-(2,2,2-trifluoroethyl)-1H-indol-4-yl)-N⁴,N⁴-dimethylcyclohexane-1,4-diamine |
| 1268. | (1R,4R)-N⁴-(2-{3-[(4-chloro-2-methoxyphenyl)amino]prop-1-yn-1-yl}-1-(2,2,2- |
| | trifluoroethyl)-1H-indol-4-yl)-N¹,N¹-dimethylcyclohexane-1,4-diamine |
| 1269. | (1S,4S)-N¹-(2-(3-((4-chloro-2-methoxyphenyl)amino)prop-1-yn-1-yl)-1-(2,2,2- |
| | trifluoroethyl)-1H-indol-4-yl)-N⁴,N⁴-dimethylcyclohexane-1,4-diamine |
| 1270. | 4-((3-(4-(((1R,4R)-4-(dimethylamino)cyclohexyl)amino)-1-(2,2,2-trifluoroethyl)-1H- |
| | indol-2-yl)prop-2-yn-1-yl)amino)-2-fluoro-5-methoxybenzamide |
| 1271. | 4-((3-(4-(((1S,4S)-4-(dimethylamino)cyclohexyl)amino)-1-(2,2,2-trifluoroethyl)-1H- |
| | indol-2-yl)prop-2-yn-1-yl)amino)-2-fluoro-5-methoxybenzamide |
| 1272. | (1R,4R)-N⁴-[2-(3-{[2-methoxy-4-(trifluoromethyl)phenyl]amino}prop-1-yn-1-yl)-1- |
| | (2,2,2-trifluoroethyl)-1H-indol-4-yl]-N¹,N¹-dimethylcyclohexane-1,4-diamine |
| 1273. | (1S,4S)-N¹-(2-(3-((2-methoxy-4-(trifluoromethyl)phenyl)amino)prop-1-yn-1-yl)-1-(2,2,2- |
| | trifluoroethyl)-1H-indol-4-yl)-N⁴,N⁴-dimethylcyclohexane-1,4-diamine |
| 1274. | 2-fluoro-5-methoxy-N-methyl-4-{[3-(4-{[(1R,4R)-4-(dimethylamino)cyclohexyl]amino}- |
| | 1-(2,2,2-trifluoroethyl)-1H-indol-2-yl)prop-2-yn-1-yl]amino}benzamide |
| 1275. | 4-((3-(4-(((1S,4S)-4-(dimethylamino)cyclohexyl)amino)-1-(2,2,2-trifluoroethyl)-1H- |
| | indol-2-yl)prop-2-yn-1-yl)amino)-2-fluoro-5-methoxy-N-methylbenzamide |
| 1276. | (1S,4S)-N⁴-[2-(3-{[4-methanesulfonyl-2-(trifluoromethyl)phenyl]amino}prop-1-yn-1-yl)- |
| | 1-(2,2,2-trifluoroethyl)-1H-indol-4-yl]-N¹,N¹-dimethylcyclohexane-1,4-diamine |
| 1277. | (1R,4R)-N⁴-[2-(3-{[4-methanesulfonyl-2-(trifluoromethyl)phenyl]amino}prop-1-yn-1-yl)- |
| | 1-(2,2,2-trifluoroethyl)-1H-indol-4-yl]-N¹,N¹-dimethylcyclohexane-1,4-diamine |
| 1278. | (1R,4R)-N⁴-(2-{3-[(4-methanesulfonyl-2-methylphenyl)amino]prop-1-yn-1-yl}-1-(2,2,2- |
| | trifluoroethyl)-1H-indol-4-yl)-N¹,N¹-dimethylcyclohexane-1,4-diamine |
| 1279. | (1S,4S)-N¹,N¹-dimethyl-N⁴-(2-(3-((2-methyl-4-(methylsulfonyl)phenyl)amino)prop-1-yn- |
| | 1-yl)-1-(2,2,2-trifluoroethyl)-1H-indol-4-yl)cyclohexane-1,4-diamine |
| 1280. | (1R,4R)-N⁴-[2-(3-{[4-methanesulfonyl-2-(trifluoromethoxy)phenyl]amino}prop-1-yn-1- |
| | yl)-1-(2,2,2-trifluoroethyl)-1H-indol-4-yl]-N¹,N¹-dimethylcyclohexane-1,4-diamine |
| 1281. | (1S,4S)-N¹,N¹-dimethyl-N⁴-(2-(3-((4-(methylsulfonyl)-2- |
| | (trifluoromethoxy)phenyl)amino)prop-1-yn-1-yl)-1-(2,2,2-trifluoroethyl)-1H-indol-4- |
| | yl)cyclohexane-1,4-diamine |
| 1282. | 3-methoxy-N-methyl-4-{[3-(4-{[(1R,4R)-4-(diethylamino)cyclohexyl]amino}-1-(2,2,2- |
| | trifluoroethyl)-1H-indol-2-yl)prop-2-yn-1-yl]amino}benzamide |
| 1283. | 4-((3-(4-(((1S,4S)-4-(diethylamino)cyclohexyl)amino)-1-(2,2,2-trifluoroethyl)-1H-indol- |
| | 2-yl)prop-2-yn-1-yl)amino)-3-methoxy-N-methylbenzamide |
| 1284. | 3-methoxy-4-{[3-(4-{[(1R,4R)-4-(diethylamino)cyclohexyl]amino}-1-(2,2,2- |
| | trifluoroethyl)-1H-indol-2-yl)prop-2-yn-1-yl]amino}benzene-1-sulfonamide |
| 1285. | 4-((3-(4-(((1S,4S)-4-(diethylamino)cyclohexyl)amino)-1-(2,2,2-trifluoroethyl)-1H-indol- |
| | 2-yl)prop-2-yn-1-yl)amino)-3-methoxybenzenesulfonamide |
| 1286. | 3-methoxy-N-methyl-4-{[3-(4-{[(1R,4R)-4-(diethylamino)cyclohexyl]amino}-1-(2,2,2- |
| | trifluoroethyl)-1H-indol-2-yl)prop-2-yn-1-yl]amino}benzene-1-sulfonamide |
| 1287. | 2-(5-methanesulfonyl-2-{[3-(4-{[(1R,4R)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}phenoxy)acetonitrile |
| 1288. | 2-(5-methanesulfonyl-2-{[3-(4-{[(1S,4S)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}phenoxy)acetonitrile |
| 1289. | 3-methoxy-N-methyl-4-{[3-(4-{[(1R,4R)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzamide |
| 1290. | 3-methoxy-N-methyl-4-{[3-(4-{[(1S,4S)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzamide |
| 1291. | N-ethyl-3-methoxy-4-{[3-(4-{[(1R,4R)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzamide |
| 1292. | N-ethyl-3-methoxy-4-((3-(4-(((1S,4S)-4-((2- |
| | methoxyethyl)(methyl)amino)cyclohexyl)amino)-1-(2,2,2-trifluoroethyl)-1H-indol-2- |
| | yl)prop-2-yn-1-yl)amino)benzamide |
| 1293. | (1R,4R)-N¹-(2-(3-((2-(fluoromethoxy)-4-(methylsulfonyl)phenyl)amino)prop-1-yn-1-yl)- |
| | 1-(2,2,2-trifluoroethyl)-1H-indol-4-yl)-N⁴-(2-methoxyethyl)-N⁴-methylcyclohexane-1,4- |
| | diamine |
| 1294. | (1S,4S)-N¹-(2-(3-((2-(fluoromethoxy)-4-(methylsulfonyl)phenyl)amino)prop-1-yn-1-yl)- |
| | 1-(2,2,2-trifluoroethyl)-1H-indol-4-yl)-N⁴-(2-methoxyethyl)-N⁴-methylcyclohexane-1,4- |
| | diamine |
| 1295. | 3-(fluoromethoxy)-N-methyl-4-{[3-(4-{[(1R,4R)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzamide |
| 1296. | 3-(fluoromethoxy)-N-methyl-4-{[3-(4-{[(1S,4S)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzamide |
| 1297. | 3-(fluoromethoxy)-4-((3-(4-(((1S,4S)-4-((2- |
| | methoxyethyl)(methyl)amino)cyclohexyl)amino)-1-(2,2,2-trifluoroethyl)-1H-indol-2- |
| | yl)prop-2-yn-1-yl)amino)-N-methylbenzamide |
| 1298. | 3-methoxy-4-{[3-(4-{[(1R,4R)-4-[(2-methoxyethyl)(methyl)amino]cyclohexyl]amino}-1- |
| | (2,2,2-trifluoroethyl)-1H-indol-2-yl)prop-2-yn-1-yl]amino}benzene-1-sulfonamide |
| 1299. | 3-methoxy-4-((3-(4-(((1S,4S)-4-((2-methoxyethyl)(methyl)amino)cyclohexyl)amino)-1- |
| | (2,2,2-trifluoroethyl)-1H-indol-2-yl)prop-2-yn-1-yl)amino)benzenesulfonamide |
| 1300. | (1R,4R)-N¹,N¹-diethyl-N⁴-(2-{3-[(4-methanesulfonyl-2-methoxyphenyl)amino]prop-1- |
| | yn-1-yl}-1-(2,2,2-trifluoroethyl)-1H-indol-4-yl)cyclohexane-1,4-diamine |
| 1301. | (1S,4S)-N¹,N¹-diethyl-N⁴-(2-(3-((2-methoxy-4-(methylsulfonyl)phenyl)amino)prop-1-yn- |
| | 1-yl)-1-(2,2,2-trifluoroethyl)-1H-indol-4-yl)cyclohexane-1,4-diamine |
| 1302. | 3-(cyanomethoxy)-4-{[3-(4-{[(1R,4R)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzene-1-sulfonamide |
| 1303. | 3-(cyanomethoxy)-4-{[3-(4-{[(1S,4S)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzene-1-sulfonamide |
| 1304. | 3-(fluoromethoxy)-4-{[3-(4-{[(1R,4R)-4-[bis(2-methoxyethyl)amino]cyclohexyl]amino}- |
| | 1-(2,2,2-trifluoroethyl)-1H-indol-2-yl)prop-2-yn-1-yl]amino}benzene-1-sulfonamide |
| 1305. | 3-(fluoromethoxy)-4-{[3-(4-{[(1S,4S)-4-[bis(2-methoxyethyl)amino]cyclohexyl]amino}- |
| | 1-(2,2,2-trifluoroethyl)-1H-indol-2-yl)prop-2-yn-1-yl]amino}benzene-1-sulfonamide |
| 1306. | 3-(fluoromethoxy)-4-{[3-(4-{[(1R,4R)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzene-1-sulfonamide |
| 1307. | 3-(fluoromethoxy)-4-{[3-(4-{[(1S,4S)-4-[(2- |
| | methoxyethyl)(methyl)amino]cyclohexyl]amino}-1-(2,2,2-trifluoroethyl)-1H-indol-2- |
| | yl)prop-2-yn-1-yl]amino}benzene-1-sulfonamide |
| 1308. | 3-methoxy-4-{[3-(4-{[(1R,4R)-4-(morpholin-4-yl)cyclohexyl]amino}-1-(2,2,2- |
| | trifluoroethyl)-1H-indol-2-yl)prop-2-yn-1-yl]amino}benzamide |
| 1309. | 3-methoxy-4-{[3-(4-{[(1S,4S)-4-(morpholin-4-yl)cyclohexyl]amino}-1-(2,2,2- |
| | trifluoroethyl)-1H-indol-2-yl)prop-2-yn-1-yl]amino}benzamide |
| 1310. | 3-methoxy-N-methyl-4-{[3-(4-{[(1R,4R)-4-(morpholin-4-yl)cyclohexyl]amino}-1-(2,2,2- |
| | trifluoroethyl)-1H-indol-2-yl)prop-2-yn-1-yl]amino}benzamide |
| 1311. | 3-methoxy-N-methyl-4-{[3-(4-{[(1S,4S)-4-(morpholin-4-yl)cyclohexyl]amino}-1-(2,2,2- |
| | trifluoroethyl)-1H-indol-2-yl)prop-2-yn-1-yl]amino}benzamide |
| 1312. | 2-(2-{3-[(4-methanesulfonyl-2-methoxyphenyl)amino]prop-1-yn-1-yl}-4-{[(1R,4R)-4- |
| | (morpholin-4-yl)cyclohexyl]amino}-1H-indol-1-yl)acetonitrile |
| 1313. | 4-({3-[1-(2-fluoroethyl)-4-{[(1R,4R)-4-(morpholin-4-yl)cyclohexyl]amino}-1H-indol-2- |
| | yl]prop-2-yn-1-yl}amino)-3-methoxy-N-methylbenzamide |
| 1314. | 1-(2-fluoroethyl)-2-{3-[(4-methanesulfonyl-2-methoxyphenyl)amino]prop-1-yn-1-yl}-N- |
| | [(1R,4R)-4-(morpholin-4-yl)cyclohexyl]-1H-indol-4-amine |
| 1315. | 4-({3-[1-(cyanomethyl)-4-{[(1R,4R)-4-(morpholin-4-yl)cyclohexyl]amino}-1H-indol-2- |
| | yl]prop-2-yn-1-yl}amino)-3-methoxy-N-methylbenzamide |
| 1316. | 2-{3-[(4-methanesulfonyl-2-methoxyphenyl)amino]prop-1-yn-1-yl}-1-propyl-N- |
| | [(1R,4R)-4-(morpholin-4-yl)cyclohexyl]-1H-indol-4-amine |
| 1317. | 2-{3-[(4-methanesulfonyl-2-methoxyphenyl)amino]prop-1-yn-1-yl}-1-(2-methylpropyl)- |
| | N-[(1R,4R)-4-(morpholin-4-yl)cyclohexyl]-1H-indol-4-amine |
| 1318. | 1-(2,2-difluoroethyl)-2-{3-[(4-methanesulfonyl-2-methoxyphenyl)amino]prop-1-yn-1- |
| | yl}-N-[(1R,4R)-4-(morpholin-4-yl)cyclohexyl]-1H-indol-4-amine |
| 1319. | 3-methoxy-4-((3-(4-(((1R,4R)-4-morpholinocyclohexyl)amino)-1-(2,2,2-trifluoroethyl)- |
| | 1H-indol-2-yl)prop-2-yn-1-yl)amino)benzenesulfonamide |
| 1320. | 3-methoxy-4-((3-(4-(((1S,4S)-4-morpholinocyclohexyl)amino)-1-(2,2,2-trifluoroethyl)- |
| | 1H-indol-2-yl)prop-2-yn-1-yl)amino)benzenesulfonamide |
| 1321. | N-ethyl-3-methoxy-4-{[3-(4-{[(1R,4R)-4-(morpholin-4-yl)cyclohexyl]amino}-1-(2,2,2- |
| | trifluoroethyl)-1H-indol-2-yl)prop-2-yn-1-yl]amino}benzamide |
| 1322. | N-ethyl-3-methoxy-4-((3-(4-(((1S,4S)-4-morpholinocyclohexyl)amino)-1-(2,2,2- |
| | trifluoroethyl)-1H-indol-2-yl)prop-2-yn-1-yl)amino)benzamide |
| 1323. | 2-(5-methanesulfonyl-2-{[3-(4-{[(1R,4R)-4-(morpholin-4-yl)cyclohexyl]amino}-1-(2,2,2- |
| | trifluoroethyl)-1H-indol-2-yl)prop-2-yn-1-yl]amino}phenoxy)acetonitrile |
| 1324. | 2-(5-methanesulfonyl-2-{[3-(4-{[(1S,4S)-4-(morpholin-4-yl)cyclohexyl]amino}-1-(2,2,2- |
| | trifluoroethyl)-1H-indol-2-yl)prop-2-yn-1-yl]amino}phenoxy)acetonitrile |
| 1325. | 3-methoxy-N-methyl-4-{[3-(4-{[(1R,4R)-4-(morpholin-4-yl)cyclohexyl]amino}-1-(2,2,2- |
| | trifluoroethyl)-1H-indol-2-yl)prop-2-yn-1-yl]amino}benzene-1-sulfonamide |
| 1326. | 2-{3-[(4-methanesulfonyl-2-methoxyphenyl)amino]prop-1-yn-1-yl}-N-[(1R,4R)-4-(3- |
| | methanesulfonylazetidin-1-yl)cyclohexyl]-1-(2,2,2-trifluoroethyl)-1H-indol-4-amine |
| 1327. | 2-(3-((2-methoxy-4-(methylsulfonyl)phenyl)amino)prop-1-yn-1-yl)-N-((1S,4S)-4-(3- |
| | (methylsulfonyl)azetidin-1-yl)cyclohexyl)-1-(2,2,2-trifluoroethyl)-1H-indol-4-amine |
| 1328. | 3-methoxy-4-{[3-(4-{[(1R,4R)-4-(3-methanesulfonylazetidin-1-yl)cyclohexyl]amino}-1- |
| | (2,2,2-trifluoroethyl)-1H-indol-2-yl)prop-2-yn-1-yl]amino}benzene-1-sulfonamide |
| 1329. | 3-methoxy-4-((3-(4-(((1S,4S)-4-(3-(methylsulfonyl)azetidin-1-yl)cyclohexyl)amino)-1- |
| | (2,2,2-trifluoroethyl)-1H-indol-2-yl)prop-2-yn-1-yl)amino)benzenesulfonamide |
| 1330. | 3-methoxy-N-methyl-4-{[3-(4-{[(1R,4R)-4-(3-methanesulfonylazetidin-1- |
| | yl)cyclohexyl]amino}-1-(2,2,2-trifluoroethyl)-1H-indol-2-yl)prop-2-yn-1- |
| | yl]amino}benzamide |
| 1331. | 3-methoxy-N-methyl-4-((3-(4-(((1S,4S)-4-(3-(methylsulfonyl)azetidin-1- |
| | yl)cyclohexyl)amino)-1-(2,2,2-trifluoroethyl)-1H-indol-2-yl)prop-2-yn-1- |
| | yl)amino)benzamide |
| 1332. | 2-{3-[(4-methanesulfonyl-2-methoxyphenyl)amino]prop-1-yn-1-yl}-N-[(1R,4R)-4-{2- |
| | azaspiro[3.3]heptan-2-yl}cyclohexyl]-1-(2,2,2-trifluoroethyl)-1H-indol-4-amine |
| 1333. | N-((1S,4S)-4-(2-azaspiro[3.3]heptan-2-yl)cyclohexyl)-2-(3-((2-methoxy-4- |
| | (methylsulfonyl)phenyl)amino)prop-1-yn-1-yl)-1-(2,2,2-trifluoroethyl)-1H-indol-4-amine |
| 1334. | 3-methoxy-N-methyl-4-{[3-(4-{[(1R,4R)-4-{2-azaspiro[3.3]heptan-2- |
| | yl}cyclohexyl]amino}-1-(2,2,2-trifluoroethyl)-1H-indol-2-yl)prop-2-yn-1- |
| | yl]amino}benzamide |
| 1335. | 4-((3-(4-(((1S,4S)-4-(2-azaspiro[3.3]heptan-2-yl)cyclohexyl)amino)-1-(2,2,2- |
| | trifluoroethyl)-1H-indol-2-yl)prop-2-yn-1-yl)amino)-3-methoxy-N-methylbenzamide |
| 1336. | 3-methoxy-4-{[3-(4-{[(1R,4R)-4-{2-azaspiro[3.3]heptan-2-yl}cyclohexyl]amino}-1- |
| | (2,2,2-trifluoroethyl)-1H-indol-2-yl)prop-2-yn-1-yl]amino}benzene-1-sulfonamide |
| 1337. | 4-((3-(4-(((1S,4S)-4-(2-azaspiro[3.3]heptan-2-yl)cyclohexyl)amino)-1-(2,2,2- |
| | trifluoroethyl)-1H-indol-2-yl)prop-2-yn-1-yl)amino)-3-methoxybenzenesulfonamide |
| 1338. | 2-(3-((2-methoxy-4-(methylsulfonyl)phenyl)amino)prop-1-yn-1-yl)-N-(2- |
| | azaspiro[3.3]heptan-6-yl)-1-(2,2,2-trifluoroethyl)-1H-indol-4-amine |
| 1339. | 4-((3-(4-((2-azaspiro[3.3]heptan-6-yl)amino)-1-(2,2,2-trifluoroethyl)-1H-indol-2-yl)prop- |
| | 2-yn-1-yl)amino)-3-methoxybenzenesulfonamide |
| 1340. | 2-{3-[(4-methanesulfonyl-2-methoxyphenyl)amino]prop-1-yn-1-yl}-N-[(1R,4R)-4-{2- |
| | oxa-6-azaspiro[3.3]heptan-6-yl}cyclohexyl]-1-(2,2,2-trifluoroethyl)-1H-indol-4-amine |
| 1341. | 2-{3-[(4-methanesulfonyl-2-methoxyphenyl)amino]prop-1-yn-1-yl}-N-[(1S,4S)-4-{2- |
| | oxa-6-azaspiro[3.3]heptan-6-yl}cyclohexyl]-1-(2,2,2-trifluoroethyl)-1H-indol-4-amine |
| 1342. | 3-methoxy-4-{[3-(4-{[(1S,4S)-4-{2-oxa-6-azaspiro[3.3]heptan-6-yl}cyclohexyl]amino}- |
| | 1-(2,2,2-trifluoroethyl)-1H-indol-2-yl)prop-2-yn-1-yl]amino}benzene-1-sulfonamide |
| 1343. | 3-methoxy-4-{[3-(4-{[(1R,4R)-4-{2-oxa-6-azaspiro[3.3]heptan-6-yl}cyclohexyl]amino}- |
| | 1-(2,2,2-trifluoroethyl)-1H-indol-2-yl)prop-2-yn-1-yl]amino}benzene-1-sulfonamide |
| 1344. | N-(3-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzenesulfonyl)propanamide |
| 1345. | N-(3-methoxy-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzenesulfonyl)propanamide |
| 1346. | N-(3-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzenesulfonyl)acetamide |
| 1347. | N-(3-methoxy-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzenesulfonyl)acetamide |
| 1348. | N-((1R,4R)-4-(2-oxa-6-azaspiro[3.3]heptan-6-yl)cyclohexyl)-2-(3-((2-(2-fluoroethoxy)-4- |
| | (methylsulfonyl)phenyl)amino)prop-1-yn-1-yl)-1-(2,2,2-trifluoroethyl)-1H-indol-4-amine |
| 1349. | N-((1S,4S)-4-(2-oxa-6-azaspiro[3.3]heptan-6-yl)cyclohexyl)-2-(3-((2-(2-fluoroethoxy)-4- |
| | (methylsulfonyl)phenyl)amino)prop-1-yn-1-yl)-1-(2,2,2-trifluoroethyl)-1H-indol-4-amine |
| 1350. | 3-methoxy-4-{[3-(4-{[(1R,4R)-4-{2-oxa-6-azaspiro[3.3]heptan-6-yl}cyclohexyl]amino}- |
| | 1-(2,2,2-trifluoroethyl)-1H-indol-2-yl)prop-2-yn-1-yl]amino}benzamide |
| 1351. | 3-methoxy-4-{[3-(4-{[(1S,4S)-4-{2-oxa-6-azaspiro[3.3]heptan-6-yl}cyclohexyl]amino}- |
| | 1-(2,2,2-trifluoroethyl)-1H-indol-2-yl)prop-2-yn-1-yl]amino}benzamide |
| 1352. | 5-methanesulfonyl-2-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenol |
| 1353. | 5-methanesulfonyl-2-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenol |
| 1354. | N-(3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)-N-(2-hydroxy-4- |
| | (methylsulfonyl)phenyl)propionamide |
| 1355. | N-(3-(4-(((1R,4R)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)-N-(2-hydroxy-4- |
| | (methylsulfonyl)phenyl)propionamide |
| 1356. | N-(2-hydroxy-4-methanesulfonylphenyl)-2-methyl-N-[3-(4-{[(1R,4R)-4-{2-oxa-6- |
| | azaspiro[3.3]heptan-6-yl}cyclohexyl]amino}-1-(2,2,2-trifluoroethyl)-1H-indol-2-yl)prop- |
| | 2-yn-1-yl]propanamide |
| 1357. | N-(3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)-N-(2-hydroxy-4- |
| | (methylsulfonyl)phenyl)isobutyramide |
| 1358. | 2-(3-{[4-methanesulfonyl-2-(2-methoxyethoxy)phenyl]amino}prop-1-yn-1-yl)-N- |
| | [(1R,4R)-4-{2-oxa-6-azaspiro[3.3]heptan-6-yl}cyclohexyl]-1-(2,2,2-trifluoroethyl)-1H- |
| | indol-4-amine |
| 1359. | 2-(3-{[4-methanesulfonyl-2-(2-methoxyethoxy)phenyl]amino}prop-1-yn-1-yl)-N- |
| | [(1S,4S)-4-{2-oxa-6-azaspiro[3.3]heptan-6-yl}cyclohexyl]-1-(2,2,2-trifluoroethyl)-1H- |
| | indol-4-amine |
| 1360. | 2-(5-methanesulfonyl-2-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenoxy)acetonitrile |
| 1361. | 2-(5-methanesulfonyl-2-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenoxy)acetonitrile |
| 1362. | 5-methanesulfonyl-2-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenyl 2-methylpropanoate |
| 1363. | 2-(5-methanesulfonyl-2-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenoxy)ethan-1-ol |
| 1364. | 2-(5-methanesulfonyl-2-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenoxy)ethan-1-ol |
| 1365. | N-(3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)-N-(2-hydroxy-4- |
| | (methylsulfonyl)phenyl)acetamide |
| 1366. | 5-methanesulfonyl-2-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenyl 2-methylpropanoate |
| 1367. | 3-methoxy-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1368. | 3-methoxy-N-methyl-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1369. | 2-(3-{[4-methanesulfonyl-2-(2,2,2-trifluoroethoxy)phenyl]amino}prop-1-yn-1-yl)-N- |
| | [(1R,4R)-4-{2-oxa-6-azaspiro[3.3]heptan-6-yl}cyclohexyl]-1-(2,2,2-trifluoroethyl)-1H- |
| | indol-4-amine |
| 1370. | 2-(3-{[4-methanesulfonyl-2-(2,2,2-trifluoroethoxy)phenyl]amino}prop-1-yn-1-yl)-N- |
| | [(1S,4S)-4-{2-oxa-6-azaspiro[3.3]heptan-6-yl}cyclohexyl]-1-(2,2,2-trifluoroethyl)-1H- |
| | indol-4-amine |
| 1371. | 2-[3-(2-amino-4-methanesulfonylphenoxy)prop-1-yn-1-yl]-N-[(1S,4S)-4-{2-oxa-6- |
| | azaspiro[3.3]heptan-6-yl}cyclohexyl]-1-(2,2,2-trifluoroethyl)-1H-indol-4-amine |
| 1372. | 2-{3-[(2-ethoxy-4-methanesulfonylphenyl)amino]prop-1-yn-1-yl}-N-[(1R,4R)-4-{2-oxa- |
| | 6-azaspiro[3.3]heptan-6-yl}cyclohexyl]-1-(2,2,2-trifluoroethyl)-1H-indol-4-amine |
| 1373. | 2-{3-[(2-ethoxy-4-methanesulfonylphenyl)amino]prop-1-yn-1-yl}-N-[(1S,4S)-4-{2-oxa- |
| | 6-azaspiro[3.3]heptan-6-yl}cyclohexyl]-1-(2,2,2-trifluoroethyl)-1H-indol-4-amine |
| 1374. | 3-(2-methoxyethoxy)-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1375. | 3-(2-methoxyethoxy)-N-methyl-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1376. | N-ethyl-3-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1377. | N-ethyl-3-methoxy-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1378. | 3-(2-fluoroethoxy)-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1379. | 3-(2-fluoroethoxy)-N-methyl-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1380. | 3-(cyanomethoxy)-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1381. | 3-(cyanomethoxy)-N-methyl-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1382. | 2-{3-[4-methanesulfonyl-2-(methylamino)phenoxy]prop-1-yn-1-yl}-N-[(1S,4S)-4-{2- |
| | oxa-6-azaspiro[3.3]heptan-6-yl}cyclohexyl]-1-(2,2,2-trifluoroethyl)-1H-indol-4-amine |
| 1383. | 2-{3-[4-methanesulfonyl-2-(methylamino)phenoxy]prop-1-yn-1-yl}-N-[(1R,4R)-4-{2- |
| | oxa-6-azaspiro[3.3]heptan-6-yl}cyclohexyl]-1-(2,2,2-trifluoroethyl)-1H-indol-4-amine |
| 1384. | 2-[3-(2-amino-4-methanesulfonylphenoxy)prop-1-yn-1-yl]-N-[(1R,4R)-4-{2-oxa-6- |
| | azaspiro[3.3]heptan-6-yl}cyclohexyl]-1-(2,2,2-trifluoroethyl)-1H-indol-4-amine |
| 1385. | N-((1R,4R)-4-(2-oxa-6-azaspiro[3.3]heptan-6-yl)cyclohexyl)-2-(3-(2-amino-4- |
| | (methylsulfonyl)phenoxy)prop-1-yn-1-yl)-1-(2,2,2-trifluoroethyl)-1H-indol-4-amine |
| 1386. | 3-hydroxy-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1387. | 3-methoxy-4-[(3-{4-[(4-{2-oxa-6-azaspiro[3.3]heptan-6-yl}cyclohexyl)amino]-1-(2,2,2- |
| | trifluoroethyl)-1H-indol-2-yl}prop-2-yn-1-yl)amino]benzonitrile |
| 1388. | 3-methoxy-4-{[3-(4-{[(1S,4S)-4-{2-oxa-6-azaspiro[3.3]heptan-6-yl}cyclohexyl]amino}- |
| | 1-(2,2,2-trifluoroethyl)-1H-indol-2-yl)prop-2-yn-1-yl]amino}benzonitrile |
| 1389. | 2-(3-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}phenyl)-2-methylpropanenitrile |
| 1390. | 2-(4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-methoxyphenyl)-2- |
| | methylpropanenitrile |
| 1391. | 3-(fluoromethoxy)-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1392. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-(fluoromethoxy)-N- |
| | methylbenzamide |
| 1393. | 3-(2-cyanoethoxy)-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1394. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-(2-cyanoethoxy)-N- |
| | methylbenzamide |
| 1395. | 2-(3-{[2-(fluoromethoxy)-4-methanesulfonylphenyl]amino}prop-1-yn-1-yl)-N-[(1R,4R)- |
| | 4-{2-oxa-6-azaspiro[3.3]heptan-6-yl}cyclohexyl]-1-(2,2,2-trifluoroethyl)-1H-indol-4- |
| | amine |
| 1396. | N-((1S,4S)-4-(2-oxa-6-azaspiro[3.3]heptan-6-yl)cyclohexyl)-2-(3-((2-(fluoromethoxy)-4- |
| | (methylsulfonyl)phenyl)amino)prop-1-yn-1-yl)-1-(2,2,2-trifluoroethyl)-1H-indol-4-amine |
| 1397. | 3-(cyanomethoxy)-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzene-1-sulfonamide |
| 1398. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3- |
| | (cyanomethoxy)benzenesulfonamide |
| 1399. | 3-(fluoromethoxy)-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzene-1-sulfonamide |
| 1400. | 3-(fluoromethoxy)-4-{[3-(4-{[(1S,4S)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzene-1-sulfonamide |
| 1401. | 4-((3-(4-(((1R,4R)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-methoxy-N- |
| | methylbenzenesulfonamide |
| 1402. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-methoxy-N- |
| | methylbenzenesulfonamide |
| 1403. | N-((1R,4R)-4-(2-oxa-6-azaspiro[3.3]heptan-6-yl)cyclohexyl)-2-(3-((4-chloro-2- |
| | methoxyphenyl)amino)prop-1-yn-1-yl)-1-(2,2,2-trifluoroethyl)-1H-indol-4-amine |
| 1404. | N-((1S,4S)-4-(2-oxa-6-azaspiro[3.3]heptan-6-yl)cyclohexyl)-2-(3-((4-chloro-2- |
| | methoxyphenyl)amino)prop-1-yn-1-yl)-1-(2,2,2-trifluoroethyl)-1H-indol-4-amine |
| 1405. | 4-((3-(4-(((1R,4R)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-methoxy-N,N- |
| | dimethylbenzenesulfonamide |
| 1406. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-methoxy-N,N- |
| | dimethylbenzenesulfonamide |
| 1407. | N-((1R,4R)-4-(2-oxa-6-azaspiro[3.3]heptan-6-yl)cyclohexyl)-2-(3-((4-(ethylsulfonyl)-2- |
| | methoxyphenyl)amino)prop-1-yn-1-yl)-1-(2,2,2-trifluoroethyl)-1H-indol-4-amine |
| 1408. | N-((1S,4S)-4-(2-oxa-6-azaspiro[3.3]heptan-6-yl)cyclohexyl)-2-(3-((4-(ethylsulfonyl)-2- |
| | methoxyphenyl)amino)prop-1-yn-1-yl)-1-(2,2,2-trifluoroethyl)-1H-indol-4-amine |
| 1409. | N-((1R,4R)-4-(2-oxa-6-azaspiro[3.3]heptan-6-yl)cyclohexyl)-2-(3-(((3S,4R)-3- |
| | methoxytetrahydro-2H-pyran-4-yl)amino)prop-1-yn-1-yl)-1-(2,2,2-trifluoroethyl)-1H- |
| | indol-4-amine |
| 1410. | N-((1S,4S)-4-(2-oxa-6-azaspiro[3.3]heptan-6-yl)cyclohexyl)-2-(3-(((3S,4R)-3- |
| | methoxytetrahydro-2H-pyran-4-yl)amino)prop-1-yn-1-yl)-1-(2,2,2-trifluoroethyl)-1H- |
| | indol-4-amine |
| 1411. | 2-fluoro-5-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1412. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-2-fluoro-5-methoxybenzamide |
| 1413. | 2-fluoro-5-methoxy-N-methyl-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzamide |
| 1414. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-2-fluoro-5-methoxy-N- |
| | methylbenzamide |
| 1415. | 2-(3-{[2-methoxy-4-(trifluoromethyl)phenyl]amino}prop-1-yn-1-yl)-N-[(1R,4R)-4-{2- |
| | oxa-6-azaspiro[3.3]heptan-6-yl}cyclohexyl]-1-(2,2,2-trifluoroethyl)-1H-indol-4-amine |
| 1416. | N-((1S,4S)-4-(2-oxa-6-azaspiro[3.3]heptan-6-yl)cyclohexyl)-2-(3-((2-methoxy-4- |
| | (trifluoromethyl)phenyl)amino)prop-1-yn-1-yl)-1-(2,2,2-trifluoroethyl)-1H-indol-4-amine |
| 1417. | 4-((3-(4-(((1R,4R)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-methoxybenzoic acid |
| 1418. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-3-methoxybenzoic acid |
| 1419. | 2-fluoro-5-methoxy-4-{[3-(4-{[(1R,4R)-4-{2-oxa-6-azaspiro[3.3]heptan-6- |
| | yl}cyclohexyl]amino}-1-(2,2,2-trifluoroethyl)-1H-indol-2-yl)prop-2-yn-1- |
| | yl]amino}benzoic acid |
| 1420. | 4-((3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)amino)-2-fluoro-5-methoxybenzoic acid |
| 1421. | 2-{3-[(4-methanesulfonyl-2-methylphenyl)amino]prop-1-yn-1-yl}-N-[(1R,4R)-4-{2-oxa- |
| | 6-azaspiro[3.3]heptan-6-yl}cyclohexyl]-1-(2,2,2-trifluoroethyl)-1H-indol-4-amine |
| 1422. | N-((1S,4S)-4-(2-oxa-6-azaspiro[3.3]heptan-6-yl)cyclohexyl)-2-(3-((2-methyl-4- |
| | (methylsulfonyl)phenyl)amino)prop-1-yn-1-yl)-1-(2,2,2-trifluoroethyl)-1H-indol-4-amine |
| 1423. | 2-(3-{[4-methanesulfonyl-2-(trifluoromethoxy)phenyl]amino}prop-1-yn-1-yl)-N- |
| | [(1R,4R)-4-{2-oxa-6-azaspiro[3.3]heptan-6-yl}cyclohexyl]-1-(2,2,2-trifluoroethyl)-1H- |
| | indol-4-amine |
| 1424. | N-((1S,4S)-4-(2-oxa-6-azaspiro[3.3]heptan-6-yl)cyclohexyl)-2-(3-((4-(methylsulfonyl)-2- |
| | (trifluoromethoxy)phenyl)amino)prop-1-yn-1-yl)-1-(2,2,2-trifluoroethyl)-1H-indol-4- |
| | amine |
| 1425. | 2-(3-{[4-methanesulfonyl-2-(trifluoromethyl)phenyl]amino}prop-1-yn-1-yl)-N-[(1R,4R)- |
| | 4-{2-oxa-6-azaspiro[3.3]heptan-6-yl}cyclohexyl]-1-(2,2,2-trifluoroethyl)-1H-indol-4- |
| | amine |
| 1426. | N-((1S,4S)-4-(2-oxa-6-azaspiro[3.3]heptan-6-yl)cyclohexyl)-2-(3-((4-(methylsulfonyl)-2- |
| | (trifluoromethyl)phenyl)amino)prop-1-yn-1-yl)-1-(2,2,2-trifluoroethyl)-1H-indol-4-amine |
| 1427. | 2-{3-[(2-chloro-4-methanesulfonylphenyl)amino]prop-1-yn-1-yl}-N-[(1R,4R)-4-{2-oxa- |
| | 6-azaspiro[3.3]heptan-6-yl}cyclohexyl]-1-(2,2,2-trifluoroethyl)-1H-indol-4-amine |
| 1428. | N-((1S,4S)-4-(2-oxa-6-azaspiro[3.3]heptan-6-yl)cyclohexyl)-2-(3-((2-chloro-4- |
| | (methylsulfonyl)phenyl)amino)prop-1-yn-1-yl)-1-(2,2,2-trifluoroethyl)-1H-indol-4-amine |
| 1429. | 3-(3-(4-(((1R,4R)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)-6-(methylsulfonyl)benzo[d]oxazol-2(3H)-one |
| 1430. | 3-(3-(4-(((1S,4S)-4-(2-oxa-6-azaspiro[3.3]heptan-6-yl)cyclohexyl)amino)-1-(2,2,2- |
| | trifluoroethyl)-1H-indol-2-yl)prop-2-yn-1-yl)-6-(methylsulfonyl)benzo[d]oxazol-2(3H)-one |
| 1431. | 2-{3-[(4-methanesulfonyl-2-methoxyphenyl)amino]prop-1-yn-1-yl}-N-[(1S,4S)-4-{7- |
| | oxa-2-azaspiro[3.5]nonan-2-yl}cyclohexyl]-1-(2,2,2-trifluoroethyl)-1H-indol-4-amine |
| 1432. | 2-{3-[(4-methanesulfonyl-2-methoxyphenyl)amino]prop-1-yn-1-yl}-N-[(1R,4R)-4-{7- |
| | oxa-2-azaspiro[3.5]nonan-2-yl}cyclohexyl]-1-(2,2,2-trifluoroethyl)-1H-indol-4-amine |
| 1433. | 3-methoxy-N-methyl-4-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzamide |
| 1434. | 3-methoxy-N-methyl-4-{[3-(4-{[(1S,4S)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzamide |
| 1435. | 3-methoxy-4-{[3-(4-{[(1R,4R)-4-{7-oxa-2-azaspiro[3.5]nonan-2-yl}cyclohexyl]amino}- |
| | 1-(2,2,2-trifluoroethyl)-1H-indol-2-yl)prop-2-yn-1-yl]amino}benzene-1-sulfonamide |
| 1436. | 3-methoxy-4-{[3-(4-{[(1S,4S)-4-{7-oxa-2-azaspiro[3.5]nonan-2-yl}cyclohexyl]amino}- |
| | 1-(2,2,2-trifluoroethyl)-1H-indol-2-yl)prop-2-yn-1-yl]amino}benzene-1-sulfonamide |
| 1437. | 3-(cyanomethoxy)-4-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzene-1-sulfonamide |
| 1438. | 3-(cyanomethoxy)-4-{[3-(4-{[(1S,4S)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzene-1-sulfonamide |
| 1439. | 2-(3-methoxy-4-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}phenyl)-2-methylpropanenitrile |
| 1440. | 2-(4-((3-(4-(((1S,4S)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxyphenyl)-2- |
| | methylpropanenitrile |
| 1441. | 2-(5-methanesulfonyl-2-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}phenoxy)acetonitrile |
| 1442. | 2-(5-methanesulfonyl-2-{[3-(4-{[(1S,4S)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}phenoxy)acetonitrile |
| 1443. | N-((4-((3-(4-(((1R,4R)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3- |
| | methoxyphenyl)sulfonyl)acetamide |
| 1444. | N-((4-((3-(4-(((1S,4S)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3- |
| | methoxyphenyl)sulfonyl)acetamide |
| 1445. | N-((4-((3-(4-(((1R,4R)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxyphenyl)sulfonyl)-2- |
| | aminoacetamide |
| 1446. | N-((4-((3-(4-(((1S,4S)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxyphenyl)sulfonyl)-2- |
| | aminoacetamide |
| 1447. | methyl 2-(5-methanesulfonyl-2-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}phenoxy)acetate |
| 1448. | methyl 2-(2-((3-(4-(((1S,4S)-4-(7-oxa-2-azaspiro[3.5]nonan-2-yl)cyclohexyl)amino)-1- |
| | (2,2,2-trifluoroethyl)-1H-indol-2-yl)prop-2-yn-1-yl)amino)-5- |
| | (methylsulfonyl)phenoxy)acetate |
| 1449. | 2-(5-methanesulfonyl-2-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}phenoxy)acetic acid |
| 1450. | 2-(2-((3-(4-(((1S,4S)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-5-(methylsulfonyl)phenoxy)acetic acid |
| 1451. | 2-{3-[(4-methanesulfonyl-2-methoxyphenyl)amino]prop-1-yn-1-yl}-N-[(1R,4R)-4-{2- |
| | oxa-7-azaspiro[3.5]nonan-7-yl}cyclohexyl]-1-(2,2,2-trifluoroethyl)-1H-indol-4-amine |
| 1452. | 2-(3-{[4-(ethanesulfonyl)-2-methoxyphenyl]amino}prop-1-yn-1-yl)-N-[(1R,4R)-4-{7- |
| | oxa-2-azaspiro[3.5]nonan-2-yl}cyclohexyl]-1-(2,2,2-trifluoroethyl)-1H-indol-4-amine |
| 1453. | 2-(3-{[4-(ethanesulfonyl)-2-methoxyphenyl]amino}prop-1-yn-1-yl)-N-[(1S,4S)-4-{7-oxa- |
| | 2-azaspiro[3.5]nonan-2-yl}cyclohexyl]-1-(2,2,2-trifluoroethyl)-1H-indol-4-amine |
| 1454. | 3-(fluoromethoxy)-4-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzene-1-sulfonamide |
| 1455. | 3-(fluoromethoxy)-4-{[3-(4-{[(1S,4S)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzene-1-sulfonamide |
| 1456. | 3-methoxy-N-methyl-4-{[3-(4-{[(1R,4R)-4-{6-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzamide |
| 1457. | 4-((3-(4-(((1S,4S)-4-(6-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxy-N-methylbenzamide |
| 1458. | 3-methoxy-4-{[3-(4-{[(1R,4R)-4-{6-oxa-2-azaspiro[3.5]nonan-2-yl}cyclohexyl]amino}- |
| | 1-(2,2,2-trifluoroethyl)-1H-indol-2-yl)prop-2-yn-1-yl]amino}benzene-1-sulfonamide |
| 1459. | 4-((3-(4-(((1S,4S)-4-(6-oxa-2-azaspiro[3.5]nonan-2-yl)cyclohexyl]amino)-1-(2,2,2- |
| | trifluoroethyl)-1H-indol-2-yl)prop-2-yn-1-yl)amino)-3-methoxybenzenesulfonamide |
| 1460. | 2-{3-[(4-methanesulfonyl-2-methoxyphenyl)amino]prop-1-yn-1-yl}-N-[(1R,4R)-4-{6- |
| | oxa-2-azaspiro[3.5]nonan-2-yl}cyclohexyl]-1-(2,2,2-trifluoroethyl)-1H-indol-4-amine |
| 1461. | N-((1S,4S)-4-(6-oxa-2-azaspiro[3.5]nonan-2-yl)cyclohexyl)-2-(3-((2-methoxy-4- |
| | (methylsulfonyl)phenyl)amino)prop-1-yn-1-yl)-1-(2,2,2-trifluoroethyl)-1H-indol-4-amine |
| 1462. | N-((1R,4R)-4-(7-oxa-2-azaspiro[3.5]nonan-2-yl)cyclohexyl)-2-(3-((2-(fluoromethoxy)-4- |
| | (methylsulfonyl)phenyl)amino)prop-1-yn-1-yl)-1-(2,2,2-trifluoroethyl)-1H-indol-4-amine |
| 1463. | N-((1S,4S)-4-(7-oxa-2-azaspiro[3.5]nonan-2-yl)cyclohexyl)-2-(3-((2-(fluoromethoxy)-4- |
| | (methylsulfonyl)phenyl)amino)prop-1-yn-1-yl)-1-(2,2,2-trifluoroethyl)-1H-indol-4-amine |
| 1464. | 4-(3-(4-(((1R,4R)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)-7-(methylsulfonyl)-2H- |
| | benzo[b][1,4]oxazin-3(4H)-one |
| 1465. | 4-((3-(4-(((1R,4R)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxy-N- |
| | methylbenzenesulfonamide |
| 1466. | 4-((3-(4-(((1S,4S)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxy-N- |
| | methylbenzenesulfonamide |
| 1467. | N-((1R,4R)-4-(7-oxa-2-azaspiro[3.5]nonan-2-yl)cyclohexyl)-2-(3-((2-ethoxy-4- |
| | (methylsulfonyl)phenyl)amino)prop-1-yn-1-yl)-1-(2,2,2-trifluoroethyl)-1H-indol-4-amine |
| 1468. | N-((1S,4S)-4-(7-oxa-2-azaspiro[3.5]nonan-2-yl)cyclohexyl)-2-(3-((2-ethoxy-4- |
| | (methylsulfonyl)phenyl)amino)prop-1-yn-1-yl)-1-(2,2,2-trifluoroethyl)-1H-indol-4-amine |
| 1469. | 3-methoxy-4-{[3-(4-{[(1R,4R)-4-{7-oxa-2-azaspiro[3.5]nonan-2-yl}cyclohexyl]amino}- |
| | 1-(2,2,2-trifluoroethyl)-1H-indol-2-yl)prop-2-yn-1-yl]amino}benzamide |
| 1470. | 4-((3-(4-(((1S,4S)-4-(7-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxybenzamide |
| 1471. | 3-methoxy-4-{[3-(4-{[(1R,4R)-4-{6-oxa-2-azaspiro[3.5]nonan-2-yl}cyclohexyl]amino}- |
| | 1-(2,2,2-trifluoroethyl)-1H-indol-2-yl)prop-2-yn-1-yl]amino}benzamide |
| 1472. | 4-((3-(4-(((1S,4S)-4-(6-oxa-2-azaspiro[3.5]nonan-2-yl)cyclohexyl)amino)-1-(2,2,2- |
| | trifluoroethyl)-1H-indol-2-yl)prop-2-yn-1-yl)amino)-3-methoxybenzamide |
| 1473. | 2-fluoro-5-methoxy-N-methyl-4-{[3-(4-{[(1R,4R)-4-{7-oxa-2-azaspiro[3.5]nonan-2- |
| | yl}cyclohexyl]amino}-1-(2,2,2-trifluoroethyl)-1H-indol-2-yl)prop-2-yn-1- |
| | yl]amino}benzamide |
| 1474. | 2-(3-((2-methoxy-4-(methylsulfonyl)phenyl)amino)prop-1-yn-1-yl)-N-((1R,4R)-4-(3- |
| | methoxypyrrolidin-1-yl)cyclohexyl)-1-(2,2,2-trifluoroethyl)-1H-indol-4-amine |
| 1475. | 2-(3-((2-methoxy-4-(methylsulfonyl)phenyl)amino)prop-1-yn-1-yl)-N-((1S,4S)-4-(3- |
| | methoxypyrrolidin-1-yl)cyclohexyl)-1-(2,2,2-trifluoroethyl)-1H-indol-4-amine |
| 1476. | 3-methoxy-4-((3-(4-(((1R,4R)-4-(3-methoxypyrrolidin-1-yl)cyclohexyl)amino)-1-(2,2,2- |
| | trifluoroethyl)-1H-indol-2-yl)prop-2-yn-1-yl)amino)benzenesulfonamide |
| 1477. | 3-methoxy-4-((3-(4-(((1S,4S)-4-(3-methoxypyrrolidin-1-yl)cyclohexyl)amino)-1-(2,2,2- |
| | trifluoroethyl)-1H-indol-2-yl)prop-2-yn-1-yl)amino)benzenesulfonamide |
| 1478. | 2-(3-{[2-(fluoromethoxy)-4-methanesulfonylphenyl]amino}prop-1-yn-1-yl)-N-[(1R,4R)- |
| | 4-(morpholin-4-yl)cyclohexyl]-1-(2,2,2-trifluoroethyl)-1H-indol-4-amine |
| 1479. | 2-(3-{[2-(fluoromethoxy)-4-methanesulfonylphenyl]amino}prop-1-yn-1-yl)-N-[(1S,4S)- |
| | 4-(morpholin-4-yl)cyclohexyl]-1-(2,2,2-trifluoroethyl)-1H-indol-4-amine |
| 1480. | 3-(cyanomethoxy)-4-{[3-(4-{[(1R,4R)-4-(morpholin-4-yl)cyclohexyl]amino}-1-(2,2,2- |
| | trifluoroethyl)-1H-indol-2-yl)prop-2-yn-1-yl]amino}benzene-1-sulfonamide |
| 1481. | 3-(cyanomethoxy)-4-{[3-(4-{[(1S,4S)-4-(morpholin-4-yl)cyclohexyl]amino}-1-(2,2,2- |
| | trifluoroethyl)-1H-indol-2-yl)prop-2-yn-1-yl]amino}benzene-1-sulfonamide |
| 1482. | 3-(fluoromethoxy)-4-{[3-(4-{[(1R,4R)-4-(morpholin-4-yl)cyclohexyl]amino}-1-(2,2,2- |
| | trifluoroethyl)-1H-indol-2-yl)prop-2-yn-1-yl]amino}benzene-1-sulfonamide |
| 1483. | 3-(fluoromethoxy)-4-{[3-(4-{[(1S,4S)-4-(morpholin-4-yl)cyclohexyl]amino}-1-(2,2,2- |
| | trifluoroethyl)-1H-indol-2-yl)prop-2-yn-1-yl]amino}benzene-1-sulfonamide |
| 1484. | 2-(3-((2-ethoxy-4-(methylsulfonyl)phenyl)amino)prop-1-yn-1-yl)-N-((1R,4R)-4- |
| | morpholinocyclohexyl)-1-(2,2,2-trifluoroethyl)-1H-indol-4-amine |
| 1485. | 2-(3-((2-ethoxy-4-(methylsulfonyl)phenyl)amino)prop-1-yn-1-yl)-N-((1S,4S)-4- |
| | morpholinocyclohexyl)-1-(2,2,2-trifluoroethyl)-1H-indol-4-amine |
| 1486. | 2-{3-[(4-methanesulfonyl-2-methoxyphenyl)amino]prop-1-yn-1-yl}-N-[(1R,4R)-4-(3- |
| | methoxypiperidin-1-yl)cyclohexyl]-1-(2,2,2-trifluoroethyl)-1H-indol-4-amine |
| 1487. | 2-(3-((2-methoxy-4-(methylsulfonyl)phenyl)amino)prop-1-yn-1-yl)-N-((1S,4S)-4-(3- |
| | methoxypiperidin-1-yl)cyclohexyl)-1-(2,2,2-trifluoroethyl)-1H-indol-4-amine |
| 1488. | 2-{3-[(4-methanesulfonyl-2-methoxyphenyl)amino]prop-1-yn-1-yl}-N-[(1R,4R)-4-(4- |
| | methoxypiperidin-1-yl)cyclohexyl]-1-(2,2,2-trifluoroethyl)-1H-indol-4-amine |
| 1489. | 2-(3-((2-methoxy-4-(methylsulfonyl)phenyl)amino)prop-1-yn-1-yl)-N-((1S,4S)-4-(4- |
| | methoxypiperidin-1-yl)cyclohexyl)-1-(2,2,2-trifluoroethyl)-1H-indol-4-amine |
| 1490. | 3-methoxy-N-methyl-4-{[3-(4-{[(1R,4R)-4-(4-methoxypiperidin-1- |
| | yl)cyclohexyl]amino}-1-(2,2,2-trifluoroethyl)-1H-indol-2-yl)prop-2-yn-1- |
| | yl]amino}benzamide |
| 1491. | 3-methoxy-4-((3-(4-(((1S,4S)-4-(4-methoxypiperidin-1-yl)cyclohexyl)amino)-1-(2,2,2- |
| | trifluoroethyl)-1H-indol-2-yl)prop-2-yn-1-yl)amino)-N-methylbenzamide |
| 1492. | 3-methoxy-4-{[3-(4-{[(1R,4R)-4-(4-methoxypiperidin-1-yl)cyclohexyl]amino}-1-(2,2,2- |
| | trifluoroethyl)-1H-indol-2-yl)prop-2-yn-1-yl]amino}benzene-1-sulfonamide |
| 1493. | 3-methoxy-4-((3-(4-(((1S,4S)-4-(4-methoxypiperidin-1-yl)cyclohexyl)amino)-1-(2,2,2- |
| | trifluoroethyl)-1H-indol-2-yl)prop-2-yn-1-yl)amino)benzenesulfonamide |
| 1494. | 3-methoxy-N-methyl-4-{[3-(4-{[(1R,4R)-4-(3-methoxypiperidin-1- |
| | yl)cyclohexyl]amino}-1-(2,2,2-trifluoroethyl)-1H-indol-2-yl)prop-2-yn-1- |
| | yl]amino}benzamide |
| 1495. | 3-methoxy-4-((3-(4-(((1S,4S)-4-(3-methoxypiperidin-1-yl)cyclohexyl)amino)-1-(2,2,2- |
| | trifluoroethyl)-1H-indol-2-yl)prop-2-yn-1-yl)amino)-N-methylbenzamide |
| 1496. | 3-methoxy-4-{[3-(4-{[(1R,4R)-4-(3-methoxypiperidin-1-yl)cyclohexyl]amino}-1-(2,2,2- |
| | trifluoroethyl)-1H-indol-2-yl)prop-2-yn-1-yl]amino}benzene-1-sulfonamide |
| 1497. | 3-methoxy-4-((3-(4-(((1S,4S)-4-(3-methoxypiperidin-1-yl)cyclohexyl)amino)-1-(2,2,2- |
| | trifluoroethyl)-1H-indol-2-yl)prop-2-yn-1-yl)amino)benzenesulfonamide |
| 1498. | 3-methoxy-4-((3-(4-(((1R,4R)-4-morpholinocyclohexyl)amino)-1-(2,2,2-trifluoroethyl)- |
| | 1H-indol-2-yl)prop-2-yn-1-yl)amino)benzenesulfonamide |
| 1499. | 3-methoxy-4-((3-(4-(((1S,4S)-4-morpholinocyclohexyl)amino)-1-(2,2,2-trifluoroethyl)- |
| | 1H-indol-2-yl)prop-2-yn-1-yl)amino)benzenesulfonamide |
| 1500. | 4-[(1R,4R)-4-[(2-{3-[(4-methanesulfonyl-2-methoxyphenyl)amino]prop-1-yn-1-yl}-1- |
| | (2,2,2-trifluoroethyl)-1H-indol-4-yl)amino]cyclohexyl]-1λ⁶-thiomorpholine-1,1-dione |
| 1501. | 4-((1S,4S)-4-((2-(3-((2-methoxy-4-(methylsulfonyl)phenyl)amino)prop-1-yn-1-yl)-1- |
| | (2,2,2-trifluoroethyl)-1H-indol-4-yl)amino)cyclohexyl)thiomorpholine 1,1-dioxide |
| 1502. | 4-[(1R,4R)-4-[(2-{3-[(4-methanesulfonyl-2-methoxyphenyl)amino]prop-1-yn-1-yl}-1- |
| | (2,2,2-trifluoroethyl)-1H-indol-4-yl)amino]cyclohexyl]-1λ⁴-thiomorpholin-1-one |
| 1503. | 4-[(1S,4S)-4-[(2-{3-[(4-methanesulfonyl-2-methoxyphenyl)amino]prop-1-yn-1-yl}-1- |
| | (2,2,2-trifluoroethyl)-1H-indol-4-yl)amino]cyclohexyl]-1λ⁴-thiomorpholin-1-one |
| 1504. | 4-((3-(4-(((1R,4R)-4-(1,1-dioxidothiomorpholino)cyclohexyl)amino)-1-(2,2,2- |
| | trifluoroethyl)-1H-indol-2-yl)prop-2-yn-1-yl)amino)-3-methoxy-N-methylbenzamide |
| 1505. | 4-((3-(4-(((1S,4S)-4-(1,1-dioxidothiomorpholino)cyclohexyl)amino)-1-(2,2,2- |
| | trifluoroethyl)-1H-indol-2-yl)prop-2-yn-1-yl)amino)-3-methoxy-N-methylbenzamide |
| 1506. | 4-((1S,4S)-4-((2-(3-((2-methoxy-4-(methylsulfonyl)phenyl)amino)prop-1-yn-1-yl)-1- |
| | (2,2,2-trifluoroethyl)-1H-indol-4-yl)amino)cyclohexyl)thiomorpholine 1-oxide |
| 1507. | 3-methoxy-N-methyl-4-{[3-(4-{[(1R,4R)-4-(1-oxo-1λ⁴-thiomorpholin-4- |
| | yl)cyclohexyl]amino}-1-(2,2,2-trifluoroethyl)-1H-indol-2-yl)prop-2-yn-1- |
| | yl]amino}benzamide |
| 1508. | 3-methoxy-4-{[3-(4-{[(1R,4R)-4-(1-oxo-1λ⁴-thiomorpholin-4-yl)cyclohexyl]amino}-1- |
| | (2,2,2-trifluoroethyl)-1H-indol-2-yl)prop-2-yn-1-yl]amino}benzene-1-sulfonamide |
| 1509. | 3-methoxy-4-((3-(4-(((1S,4S)-4-(1-oxidothiomorpholino)cyclohexyl)amino)-1-(2,2,2- |
| | trifluoroethyl)-1H-indol-2-yl)prop-2-yn-1-yl)amino)benzenesulfonamide |
| 1510. | 3-methoxy-4-{[3-(4-{[(1R,4R)-4-(1,1-dioxo-1λ⁶-thiomorpholin-4-yl)cyclohexyl]amino}- |
| | 1-(2,2,2-trifluoroethyl)-1H-indol-2-yl)prop-2-yn-1-yl]amino}benzene-1-sulfonamide |
| 1511. | 4-((3-(4-(((1S,4S)-4-(1,1-dioxidothiomorpholino)cyclohexyl)amino)-1-(2,2,2- |
| | trifluoroethyl)-1H-indol-2-yl)prop-2-yn-1-yl)amino)-3-methoxybenzenesulfonamide |
| 1512. | 2-{3-[(4-methanesulfonyl-2-methoxyphenyl)amino]prop-1-yn-1-yl}-N-[(1S,4S)-4-{2- |
| | oxa-7-azaspiro[3.5]nonan-7-yl}cyclohexyl]-1-(2,2,2-trifluoroethyl)-1H-indol-4-amine |
| 1513. | 3-methoxy-N-methyl-4-{[3-(4-{[(1R,4R)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzamide |
| 1514. | 3-methoxy-N-methyl-4-{[3-(4-{[(1S,4S)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzamide |
| 1515. | 3-methoxy-4-{[3-(4-{[(1R,4R)-4-{2-oxa-7-azaspiro[3.5]nonan-7-yl}cyclohexyl]amino}- |
| | 1-(2,2,2-trifluoroethyl)-1H-indol-2-yl)prop-2-yn-1-yl]amino}benzene-1-sulfonamide |
| 1516. | 4-((3-(4-(((1S,4S)-4-(2-oxa-7-azaspiro[3.5]nonan-7-yl)cyclohexyl)amino)-1-(2,2,2- |
| | trifluoroethyl)-1H-indol-2-yl)prop-2-yn-1-yl)amino)-3-methoxybenzenesulfonamide |
| 1517. | 3-(cyanomethoxy)-4-{[3-(4-{[(1R,4R)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1518. | 3-(cyanomethoxy)-4-{[3-(4-{[(1S,4S)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1519. | 3-(fluoromethoxy)-4-{[3-(4-{[(1R,4R)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1520. | 3-(fluoromethoxy)-4-{[3-(4-{[(1S,4S)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1521. | 2-{3-[(4-methanesulfonyl-2-methoxyphenyl)amino]prop-1-yn-1-yl}-N-[(1R,4R)-4-{1- |
| | oxa-7-azaspiro[3.5]nonan-7-yl}cyclohexyl]-1-(2,2,2-trifluoroethyl)-1H-indol-4-amine |
| 1522. | N-((1S,4S)-4-(1-oxa-7-azaspiro[3.5]nonan-7-yl)cyclohexyl)-2-(3-((2-methoxy-4- |
| | (methylsulfonyl)phenyl)amino)prop-1-yn-1-yl)-1-(2,2,2-trifluoroethyl)-1H-indol-4-amine |
| 1523. | 3-methoxy-N-methyl-4-{[3-(4-{[(1R,4R)-4-{1-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzamide |
| 1524. | 4-((3-(4-(((1S,4S)-4-(1-oxa-7-azaspiro[3.5]nonan-7-yl)cyclohexyl)amino)-1-(2,2,2- |
| | trifluoroethyl)-1H-indol-2-yl)prop-2-yn-1-yl)amino)-3-methoxy-N-methylbenzamide |
| 1525. | 3-methoxy-4-{[3-(4-{[(1R,4R)-4-{1-oxa-7-azaspiro[3.5]nonan-7-yl}cyclohexyl]amino}- |
| | 1-(2,2,2-trifluoroethyl)-1H-indol-2-yl)prop-2-yn-1-yl]amino}benzene-1-sulfonamide |
| 1526. | 4-((3-(4-(((1S,4S)-4-(1-oxa-7-azaspiro[3.5]nonan-7-yl)cyclohexyl)amino)-1-(2,2,2- |
| | trifluoroethyl)-1H-indol-2-yl)prop-2-yn-1-yl)amino)-3-methoxybenzenesulfonamide |
| 1527. | 2-(5-methanesulfonyl-2-{[3-(4-{[(1R,4R)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}phenoxy)acetonitrile |
| 1528. | 2-(5-methanesulfonyl-2-{[3-(4-{[(1S,4S)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}phenoxy)acetonitrile |
| 1529. | 2-(3-{[2-(fluoromethoxy)-4-methanesulfonylphenyl]amino}prop-1-yn-1-yl)-N-[(1R,4R)- |
| | 4-{2-oxa-7-azaspiro[3.5]nonan-7-yl}cyclohexyl]-1-(2,2,2-trifluoroethyl)-1H-indol-4- |
| | amine |
| 1530. | 2-(3-{[2-(fluoromethoxy)-4-methanesulfonylphenyl]amino}prop-1-yn-1-yl)-N-[(1S,4S)- |
| | 4-{2-oxa-7-azaspiro[3.5]nonan-7-yl}cyclohexyl]-1-(2,2,2-trifluoroethyl)-1H-indol-4- |
| | amine |
| 1531. | N-((1R,4R)-4-(2-oxa-7-azaspiro[3.5]nonan-7-yl)cyclohexyl)-2-(3-((2-ethoxy-4- |
| | (methylsulfonyl)phenyl)amino)prop-1-yn-1-yl)-1-(2,2,2-trifluoroethyl)-1H-indol-4-amine |
| 1532. | N-((1S,4S)-4-(2-oxa-7-azaspiro[3.5]nonan-7-yl)cyclohexyl)-2-(3-((2-ethoxy-4- |
| | (methylsulfonyl)phenyl)amino)prop-1-yn-1-yl)-1-(2,2,2-trifluoroethyl)-1H-indol-4-amine |
| 1533. | 3-methoxy-N-methyl-4-{[3-(4-{[(1R,4R)-4-{2-oxa-7-azaspiro[3.5]nonan-7- |
| | yl}cyclohexyl]amino}-1-(2,2,2-trifluoroethyl)-1H-indol-2-yl)prop-2-yn-1- |
| | yl]amino}benzene-1-sulfonamide |
| 1534. | N-((1R,4R)-4-(2-oxa-7-azaspiro[3.5]nonan-7-yl)cyclohexyl)-2-(3-((4-(ethylsulfonyl)-2- |
| | methoxyphenyl)amino)prop-1-yn-1-yl)-1-(2,2,2-trifluoroethyl)-1H-indol-4-amine |
| 1535. | N-((1S,4S)-4-(2-oxa-7-azaspiro[3.5]nonan-7-yl)cyclohexyl)-2-(3-((4-(ethylsulfonyl)-2- |
| | methoxyphenyl)amino)prop-1-yn-1-yl)-1-(2,2,2-trifluoroethyl)-1H-indol-4-amine |
| 1536. | 2-{3-[(4-methanesulfonyl-2-methoxyphenyl)amino]prop-1-yn-1-yl}-N-[(1R,4R)-4-{6- |
| | oxa-3-azabicyclo[3.1.1]heptan-3-yl}cyclohexyl]-1-(2,2,2-trifluoroethyl)-1H-indol-4- |
| | amine |
| 1537. | 2-{3-[(4-methanesulfonyl-2-methoxyphenyl)amino]prop-1-yn-1-yl}-N-[(1S,4S)-4-{6- |
| | oxa-3-azabicyclo[3.1.1]heptan-3-yl}cyclohexyl]-1-(2,2,2-trifluoroethyl)-1H-indol-4- |
| | amine |
| 1538. | 3-methoxy-4-{[3-(4-{[(1R,4R)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}benzene-1-sulfonamide |
| 1539. | 3-methoxy-4-{[3-(4-{[(1S,4S)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}benzene-1-sulfonamide |
| 1540. | 3-methoxy-N-methyl-4-{[3-(4-{[(1R,4R)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}benzamide |
| 1541. | 3-methoxy-N-methyl-4-{[3-(4-{[(1S,4S)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}benzamide |
| 1542. | 2-{3-[(2-ethoxy-4-methanesulfonylphenyl)amino]prop-1-yn-1-yl}-N-[(1R,4R)-4-{6-oxa- |
| | 3-azabicyclo[3.1.1]heptan-3-yl}cyclohexyl]-1-(2,2,2-trifluoroethyl)-1H-indol-4-amine |
| 1543. | 2-{3-[(2-ethoxy-4-methanesulfonylphenyl)amino]prop-1-yn-1-yl}-N-[(1S,4S)-4-{6-oxa- |
| | 3-azabicyclo[3.1.1]heptan-3-yl}cyclohexyl]-1-(2,2,2-trifluoroethyl)-1H-indol-4-amine |
| 1544. | N-ethyl-3-methoxy-4-{[3-(4-{[(1R,4R)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}benzamide |
| 1545. | 2-(5-methanesulfonyl-2-{[3-(4-{[(1R,4R)-4-{6-oxa-3-azabicyclo[3.1.1]heptan-3- |
| | yl}cyclohexyl]amino}-1-(2,2,2-trifluoroethyl)-1H-indol-2-yl)prop-2-yn-1- |
| | yl]amino}phenoxy)acetonitrile |
| 1546. | 2-(2-((3-(4-(((1S,4S)-4-(6-oxa-3-azabicyclo[3.1.1]heptan-3-yl)cyclohexyl)amino)-1- |
| | (2,2,2-trifluoroethyl)-1H-indol-2-yl)prop-2-yn-1-yl)amino)-5- |
| | (methylsulfonyl)phenoxy)acetonitrile |
| 1547. | 3-methoxy-N-methyl-4-{[3-(4-{[(1R,4R)-4-{2-oxa-8-azaspiro[4.5]decan-8- |
| | yl}cyclohexyl]amino}-1-(2,2,2-trifluoroethyl)-1H-indol-2-yl)prop-2-yn-1- |
| | yl]amino}benzamide |
| 1548. | 2-{3-[(4-methanesulfonyl-2-methoxyphenyl)amino]prop-1-yn-1-yl}-N-[(1R,4R)-4-{2- |
| | oxa-8-azaspiro[4.5]decan-8-yl}cyclohexyl]-1-(2,2,2-trifluoroethyl)-1H-indol-4-amine |
| 1549. | 3-methoxy-4-{[3-(4-{[(1R,4R)-4-{2-oxa-8-azaspiro[4.5]decan-8-yl}cyclohexyl]amino}- |
| | 1-(2,2,2-trifluoroethyl)-1H-indol-2-yl)prop-2-yn-1-yl]amino}benzene-1-sulfonamide |
| 1550. | 4-((3-(4-(((1R,4R)-4-(2-oxa-8-azaspiro[4.5]decan-8-yl)cyclohexyl)amino)-1-(2,2,2- |
| | trifluoroethyl)-1H-benzo[d]imidazol-2-yl)prop-2-yn-1-yl)amino)-3- |
| | methoxybenzenesulfonamide |
| 1551. | 4-((3-(4-(((1S,4S)-4-(2-oxa-8-azaspiro[4.5]decan-8-yl)cyclohexyl)amino)-1-(2,2,2- |
| | trifluoroethyl)-1H-benzo[d]imidazol-2-yl)prop-2-yn-1-yl)amino)-3- |
| | methoxybenzenesulfonamide |
| 1552. | 2-{3-[(4-methanesulfonyl-2-methoxyphenyl)amino]prop-1-yn-1-yl}-N-[(1R,4R)-4-{3- |
| | oxa-9-azaspiro[5.5]undecan-9-yl}cyclohexyl]-1-(2,2,2-trifluoroethyl)-1H-indol-4-amine |
| 1553. | N-((1S,4S)-4-(3-oxa-9-azaspiro[5.5]undecan-9-yl)cyclohexyl)-2-(3-((2-methoxy-4- |
| | (methylsulfonyl)phenyl)amino)prop-1-yn-1-yl)-1-(2,2,2-trifluoroethyl)-1H-indol-4-amine |
| 1554. | 3-methoxy-N-methyl-4-{[3-(4-{[(1R,4R)-4-{3-oxa-9-azaspiro[5.5]undecan-9- |
| | yl}cyclohexyl]amino}-1-(2,2,2-trifluoroethyl)-1H-indol-2-yl)prop-2-yn-1- |
| | yl]amino}benzamide |
| 1555. | 4-((3-(4-(((1S,4S)-4-(3-oxa-9-azaspiro[5.5]undecan-9-yl)cyclohexyl)amino)-1-(2,2,2- |
| | trifluoroethyl)-1H-indol-2-yl)prop-2-yn-1-yl)amino)-3-methoxy-N-methylbenzamide |
| 1556. | 3-methoxy-4-{[3-(4-{[(1R,4R)-4-{3-oxa-9-azaspiro[5.5]undecan-9- |
| | yl}cyclohexyl]amino}-1-(2,2,2-trifluoroethyl)-1H-indol-2-yl)prop-2-yn-1- |
| | yl]amino}benzene-1-sulfonamide |
| 1557. | 4-((3-(4-(((1S,4S)-4-(3-oxa-9-azaspiro[5.5]undecan-9-yl)cyclohexyl)amino)-1-(2,2,2- |
| | trifluoroethyl)-1H-indol-2-yl)prop-2-yn-1-yl)amino)-3-methoxybenzenesulfonamide |
| 1558. | 3-methoxy-N-methyl-4-{[3-(4-{[(1R,4R)-4-(pyrrolidin-1-yl)cyclohexyl]amino}-1-(2,2,2- |
| | trifluoroethyl)-1H-indol-2-yl)prop-2-yn-1-yl]amino}benzamide |
| 1559. | 3-methoxy-N-methyl-4-((3-(4-(((1S,4S)-4-(pyrrolidin-1-yl)cyclohexyl]amino)-1-(2,2,2- |
| | trifluoroethyl)-1H-indol-2-yl)prop-2-yn-1-yl)amino)benzamide |
| 1560. | 3-methoxy-4-{[3-(4-{[(1R,4R)-4-(pyrrolidin-1-yl)cyclohexyl]amino}-1-(2,2,2- |
| | trifluoroethyl)-1H-indol-2-yl)prop-2-yn-1-yl]amino}benzene-1-sulfonamide |
| 1561. | 3-methoxy-4-((3-(4-(((1S,4S)-4-(pyrrolidin-1-yl)cyclohexyl]amino)-1-(2,2,2- |
| | trifluoroethyl)-1H-indol-2-yl)prop-2-yn-1-yl)amino)benzenesulfonamide |
| 1562. | 2-{3-[(4-methanesulfonyl-2-methoxyphenyl)amino]prop-1-yn-1-yl}-N-[(1R,4R)-4- |
| | (pyrrolidin-1-yl)cyclohexyl]-1-(2,2,2-trifluoroethyl)-1H-indol-4-amine |
| 1563. | 2-(3-((2-methoxy-4-(methylsulfonyl)phenyl)amino)prop-1-yn-1-yl)-N-((1S,4S)-4- |
| | (pyrrolidin-1-yl)cyclohexyl)-1-(2,2,2-trifluoroethyl)-1H-indol-4-amine |
| 1564. | 2-{3-[(4-methanesulfonyl-2-methoxyphenyl)amino]prop-1-yn-1-yl}-N-[(1R,4R)-4-(3- |
| | methoxypyrrolidin-1-yl)cyclohexyl]-1-(2,2,2-trifluoroethyl)-1H-indol-4-amine |
| 1565. | 3-methoxy-4-{[3-(4-{[(1R,4R)-4-(3-methoxypyrrolidin-1-yl)cyclohexyl]amino}-1-(2,2,2- |
| | trifluoroethyl)-1H-indol-2-yl)prop-2-yn-1-yl]amino}benzene-1-sulfonamide |
| 1566. | 3-methoxy-N-methyl-4-{[3-(4-{[(1R,4R)-4-(3-methoxypyrrolidin-1- |
| | yl)cyclohexyl]amino}-1-(2,2,2-trifluoroethyl)-1H-indol-2-yl)prop-2-yn-1- |
| | yl]amino}benzamide |
| 1567. | 3-methoxy-4-((3-(4-(((1S,4S)-4-(3-methoxypyrrolidin-1-yl)cyclohexyl)amino)-1-(2,2,2- |
| | trifluoroethyl)-1H-indol-2-yl)prop-2-yn-1-yl)amino)-N-methylbenzamide |
| 1568. | 3-methoxy-4-{[3-(4-{[(1R,4R)-4-{hexahydro-1H-furo[3,4-c]pyrrol-5- |
| | yl}cyclohexyl]amino}-1-(2,2,2-trifluoroethyl)-1H-indol-2-yl)prop-2-yn-1- |
| | yl]amino}benzene-1-sulfonamide |
| 1569. | 3-methoxy-4-((3-(4-(((1S,4S)-4-(tetrahydro-1H-furo[3,4-c]pyrrol-5(3H)- |
| | yl)cyclohexyl)amino)-1-(2,2,2-trifluoroethyl)-1H-indol-2-yl)prop-2-yn-1- |
| | yl)amino)benzenesulfonamide |
| 1570. | 2-{3-[(4-methanesulfonyl-2-methoxyphenyl)amino]prop-1-yn-1-yl}-N-[(1R,4R)-4- |
| | {hexahydro-1H-furo[3,4-c]pyrrol-5-yl}cyclohexyl]-1-(2,2,2-trifluoroethyl)-1H-indol-4- |
| | amine |
| 1571. | 2-(3-((2-methoxy-4-(methylsulfonyl)phenyl)amino)prop-1-yn-1-yl)-N-((1R,4R)-4- |
| | (tetrahydro-1H-furo[3,4-c]pyrrol-5(3H)-yl)cyclohexyl)-1-(2,2,2-trifluoroethyl)-1H-indol- |
| | 4-amine |
| 1572. | 3-methoxy-N-methyl-4-{[3-(4-{[(1R,4R)-4-{hexahydro-1H-furo[3,4-c]pyrrol-5- |
| | yl}cyclohexyl]amino}-1-(2,2,2-trifluoroethyl)-1H-indol-2-yl)prop-2-yn-1- |
| | yl]amino}benzamide |
| 1573. | 3-methoxy-N-methyl-4-((3-(4-(((1S,4S)-4-(tetrahydro-1H-furo[3,4-c]pyrrol-5(3H)- |
| | yl)cyclohexyl)amino)-1-(2,2,2-trifluoroethyl)-1H-indol-2-yl)prop-2-yn-1- |
| | yl)amino)benzamide |
| 1574. | 2-{3-[(4-methanesulfonyl-2-methoxyphenyl)amino]prop-1-yn-1-yl}-N-[(1R,4R)-4-[4- |
| | (trifluoromethyl)piperidin-1-yl]cyclohexyl]-1-(2,2,2-trifluoroethyl)-1H-indol-4-amine |
| 1575. | 2-(3-((2-methoxy-4-(methylsulfonyl)phenyl)amino)prop-1-yn-1-yl)-1-(2,2,2- |
| | trifluoroethyl)-N-((1S,4S)-4-(4-(trifluoromethyl)piperidin-1-yl)cyclohexyl)-1H-indol-4- |
| | amine |
| 1576. | 3-methoxy-N-methyl-4-{[3-(4-{[(1R,4R)-4-[4-(trifluoromethyl)piperidin-1- |
| | yl]cyclohexyl]amino}-1-(2,2,2-trifluoroethyl)-1H-indol-2-yl)prop-2-yn-1- |
| | yl]amino}benzamide |
| 1577. | 3-methoxy-N-methyl-4-((3-(1-(2,2,2-trifluoroethyl)-4-(((1S,4S)-4-(4- |
| | (trifluoromethyl)piperidin-1-yl)cyclohexyl)amino)-1H-indol-2-yl)prop-2-yn-1- |
| | yl)amino)benzamide |
| 1578. | 3-methoxy-4-{[3-(4-{[(1R,4R)-4-[4-(trifluoromethyl)piperidin-1-yl]cyclohexyl]amino}- |
| | 1-(2,2,2-trifluoroethyl)-1H-indol-2-yl)prop-2-yn-1-yl]amino}benzene-1-sulfonamide |
| 1579. | 3-methoxy-4-((3-(1-(2,2,2-trifluoroethyl)-4-(((1S,4S)-4-(4-(trifluoromethyl)piperidin-1- |
| | yl)cyclohexyl)amino)-1H-indol-2-yl)prop-2-yn-1-yl)amino)benzenesulfonamide |
| 1580. | 2-{3-[(4-methanesulfonyl-2-methoxyphenyl)amino]prop-1-yn-1-yl}-N-[(1R,4R)-4-(4- |
| | methanesulfonylpiperidin-1-yl)cyclohexyl]-1-(2,2,2-trifluoroethyl)-1H-indol-4-amine |
| 1581. | 2-(3-((2-methoxy-4-(methylsulfonyl)phenyl)amino)prop-1-yn-1-yl)-N-((1S,4S)-4-(4- |
| | (methylsulfonyl)piperidin-1-yl)cyclohexyl)-1-(2,2,2-trifluoroethyl)-1H-indol-4-amine |
| 1582. | 3-methoxy-N-methyl-4-{[3-(4-{[(1R,4R)-4-(4-methanesulfonylpiperidin-1- |
| | yl)cyclohexyl]amino}-1-(2,2,2-trifluoroethyl)-1H-indol-2-yl)prop-2-yn-1- |
| | yl]amino}benzamide |
| 1583. | 3-methoxy-N-methyl-4-((3-(4-(((1S,4S)-4-(4-(methylsulfonyl)piperidin-1- |
| | yl)cyclohexyl)amino)-1-(2,2,2-trifluoroethyl)-1H-indol-2-yl)prop-2-yn-1- |
| | yl)amino)benzamide |
| 1584. | 3-methoxy-4-{[3-(4-{[(1R,4R)-4-(4-methanesulfonylpiperidin-1-yl)cyclohexyl]amino}-1- |
| | (2,2,2-trifluoroethyl)-1H-indol-2-yl)prop-2-yn-1-yl]amino}benzene-1-sulfonamide |
| 1585. | 3-methoxy-4-((3-(4-(((1S,4S)-4-(4-(methylsulfonyl)piperidin-1-yl)cyclohexyl)amino)-1- |
| | (2,2,2-trifluoroethyl)-1H-indol-2-yl)prop-2-yn-1-yl)amino)benzenesulfonamide |
| 1586. | 1-[(1S,3R)-3-[(2-{3-[(4-methanesulfonyl-2-methoxyphenyl)amino]prop-1-yn-1-yl}-1- |
| | (2,2,2-trifluoroethyl)-1H-indol-4-yl)amino]cyclohexyl]-4,5-dihydro-1H-1,2,3,4-tetrazol- |
| | 5-one |
| 1587. | 2-((2-(3-((2-methoxy-4-(methylsulfonyl)phenyl)amino)prop-1-yn-1-yl)-4-(((1R,4R)-4- |
| | morpholinocyclohexyl)amino)-1H-indol-1-yl)methyl)acrylonitrile |
| 1588. | N-((1R,4R)-4-(2-oxa-8-azaspiro[4.5]decan-8-yl)cyclohexyl)-2-(3-((2-methoxy-4- |
| | (methylsulfonyl)phenyl)amino)prop-1-yn-1-yl)-1-(2,2,2-trifluoroethyl)-1H- |
| | benzo[d]imidazol-4-amine |
| 1589. | N-((1S,4S)-4-(2-oxa-8-azaspiro[4.5]decan-8-yl)cyclohexyl)-2-(3-((2-methoxy-4- |
| | (methylsulfonyl)phenyl)amino)prop-1-yn-1-yl)-1-(2,2,2-trifluoroethyl)-1H- |
| | benzo[d]imidazol-4-amine |
| 1590. | 4-((3-(4-(((1R,4R)-4-(2-oxa-8-azaspiro[4.5]decan-8-yl)cyclohexyl)amino)-1-(2,2,2- |
| | trifluoroethyl)-1H-benzo[d]imidazol-2-yl)prop-2-yn-1-yl)amino)-3-methoxy-N- |
| | methylbenzamide |
| 1591. | 4-((3-(4-(((1S,4S)-4-(2-oxa-8-azaspiro[4.5]decan-8-yl)cyclohexyl)amino)-1-(2,2,2- |
| | trifluoroethyl)-1H-benzo[d]imidazol-2-yl)prop-2-yn-1-yl)amino)-3-methoxy-N- |
| | methylbenzamide |
| 1592. | 1-{3-[(2-{3-[(4-methanesulfonyl-2-methoxyphenyl)amino]prop-1-yn-1-yl}-1-(2,2,2- |
| | trifluoroethyl)-1H-indol-4-yl)amino]pyrrolidin-1-yl}-3-methoxypropan-2-ol |
| 1593. | N-[3-(4-{[1-(2-hydroxy-3-methoxypropyl)pyrrolidin-3-yl]amino}-1-(2,2,2- |
| | trifluoroethyl)-1H-indol-2-yl)prop-2-yn-1-yl]benzamide |
| 1594. | 1-{3-[(2-{3-[(4-methanesulfonylphenyl)amino]prop-1-yn-1-yl}-1-(2,2,2-trifluoroethyl)- |
| | 1H-indol-4-yl)amino]pyrrolidin-1-yl}-3-methoxypropan-2-ol |
| 1595. | 2-{3-[(4-methanesulfonyl-2-methoxyphenyl)amino]prop-1-yn-1-yl}-N-[1-(oxan-4- |
| | yl)pyrrolidin-3-yl]-1-(2,2,2-trifluoroethyl)-1H-indol-4-amine |
| 1596. | 2-(3-{[2-methoxy-4-(morpholine-4-sulfonyl)phenyl]amino}prop-1-yn-1-yl)-N-(piperidin- |
| | 4-yl)-1-(2,2,2-trifluoroethyl)-1H-indol-4-amine |
| 1597. | 3-methoxy-N,N-dimethyl-4-[(3-{4-[(piperidin-4-yl)amino]-1-(2,2,2-trifluoroethyl)-1H- |
| | indol-2-yl}prop-2-yn-1-yl)amino]benzene-1-sulfonamide |
| 1598. | 2-{5-methanesulfonyl-2-[(3-{4-[(piperidin-4-yl)amino]-1-(2,2,2-trifluoroethyl)-1H-indol- |
| | 2-yl}prop-2-yn-1-yl)amino]phenoxy}acetonitrile |
| 1599. | 3-methoxy-4-((3-(4-(piperidin-4-ylamino)-1-(2,2,2-trifluoroethyl)-1H-indol-2-yl)prop-2- |
| | yn-1-yl)amino)benzamide |
| 1600. | 2-{3-[(4-methanesulfonyl-2-methoxyphenyl)amino]prop-1-yn-1-yl}-N-[(3R)-piperidin-3- |
| | yl]-1-(2,2,2-trifluoroethyl)-1H-indol-4-amine |
| 1601. | 3-methoxy-4-{[3-(4-{[(2S,4S)-2-methylpiperidin-4-yl]amino}-1-(2,2,2-trifluoroethyl)- |
| | 1H-indol-2-yl)prop-2-yn-1-yl]amino}benzene-1-sulfonamide |
| 1602. | 3-methoxy-4-((3-(4-((1-methylpiperidin-4-yl)amino)-1-(2,2,2-trifluoroethyl)-1H-indol-2- |
| | yl)prop-2-yn-1-yl)amino)benzenesulfonamide |
| 1603. | 2-(3-{[4-methanesulfonyl-2-(trifluoromethoxy)phenyl]amino}prop-1-yn-1-yl)-N-(1- |
| | methylpiperidin-4-yl)-1-(2,2,2-trifluoroethyl)-1H-indol-4-amine |
| 1604. | N-(1-methylpiperidin-4-yl)-2-(3-((4-(methylsulfonyl)-2-(2,2,2- |
| | trifluoroethoxy)phenyl)amino)prop-1-yn-1-yl)-1-(2,2,2-trifluoroethyl)-1H-indol-4-amine |
| 1605. | 2-{4-methoxy-5-[(3-{4-[(1-methylpiperidin-4-yl)amino]-1-(2,2,2-trifluoroethyl)-1H- |
| | indol-2-yl}prop-2-yn-1-yl)amino]pyridin-2-yl}-2-methylpropanenitrile |
| 1606. | 3-methoxy-4-[(3-{4-[(1-methylpiperidin-4-yl)amino]-1-(2,2,2-trifluoroethyl)-1H-indol-2- |
| | yl}prop-2-yn-1-yl)amino]-N-(1,2-oxazol-3-yl)benzene-1-sulfonamide |
| 1607. | 3-methoxy-4-[(3-{4-[(1-methylpiperidin-4-yl)amino]-1-(2,2,2-trifluoroethyl)-1H-indol-2- |
| | yl}prop-2-yn-1-yl)amino]-N-(oxan-4-yl)benzene-1-sulfonamide |
| 1608. | 3-methoxy-N-(5-methyl-1,2-oxazol-3-yl)-4-[(3-{4-[(1-methylpiperidin-4-yl)amino]-1- |
| | (2,2,2-trifluoroethyl)-1H-indol-2-yl}prop-2-yn-1-yl)amino]benzene-1-sulfonamide |
| 1609. | N-(2-hydroxyethyl)-3-methoxy-N-methyl-4-[(3-{4-[(1-methylpiperidin-4-yl)amino]-1- |
| | (2,2,2-trifluoroethyl)-1H-indol-2-yl}prop-2-yn-1-yl)amino]benzene-1-sulfonamide |
| 1610. | 3-methoxy-N,N-dimethyl-4-[(3-{4-[(1-methylpiperidin-4-yl)amino]-1-(2,2,2- |
| | trifluoroethyl)-1H-indol-2-yl}prop-2-yn-1-yl)amino]benzene-1-sulfonamide |
| 1611. | 4-[(3-{6-fluoro-4-[(1-methylpiperidin-4-yl)amino]-1-(2,2,2-trifluoroethyl)-1H-indol-2- |
| | yl}prop-2-yn-1-yl)amino]-3-methoxybenzene-1-sulfonamide |
| 1612. | 6-fluoro-2-{3-[(4-methanesulfonyl-2-methoxyphenyl)amino]prop-1-yn-1-yl}-N-(1- |
| | methylpiperidin-4-yl)-1-(2,2,2-trifluoroethyl)-1H-indol-4-amine |
| 1613. | 2-(3-((4-methoxy-6-(methylsulfonyl)pyridin-3-yl)amino)prop-1-yn-1-yl)-N-(1- |
| | methylpiperidin-4-yl)-1-(2,2,2-trifluoroethyl)-1H-indol-4-amine |
| 1614. | 3-methoxy-N-(2-methoxyethyl)-N-methyl-4-[(3-{4-[(1-methylpiperidin-4-yl)amino]-1- |
| | (2,2,2-trifluoroethyl)-1H-indol-2-yl}prop-2-yn-1-yl)amino]benzene-1-sulfonamide |
| 1615. | 2-{3-[(4-methanesulfonyl-2-methoxyphenyl)(methyl)amino]prop-1-yn-1-yl}-N-(1- |
| | methylpiperidin-4-yl)-1-(2,2,2-trifluoroethyl)-1H-indol-4-amine |
| 1616. | 1-(4-{3-methoxy-4-[(3-{4-[(1-methylpiperidin-4-yl)amino]-1-(2,2,2-trifluoroethyl)-1H- |
| | indol-2-yl}prop-2-yn-1-yl)amino]benzenesulfonyl}piperazin-1-yl)ethan-1-one |
| 1617. | 2-(3-{[2-methoxy-4-(morpholine-4-sulfonyl)phenyl]amino}prop-1-yn-1-yl)-N-(1- |
| | methylpiperidin-4-yl)-1-(2,2,2-trifluoroethyl)-1H-indol-4-amine |
| 1618. | 3-methoxy-4-((3-(4-((1-methylpiperidin-4-yl)amino)-1-(2,2,2-trifluoroethyl)-1H-indol-2- |
| | yl)prop-2-yn-1-yl)amino)benzoic acid |
| 1619. | 3-methoxy-N-methyl-4-[(3-{4-[(1-methylpiperidin-4-yl)amino]-1-(2,2,2-trifluoroethyl)- |
| | 1H-indol-2-yl}prop-2-yn-1-yl)amino]benzamide |
| 1620. | N,N-bis(2-hydroxyethyl)-3-methoxy-4-[(3-{4-[(1-methylpiperidin-4-yl)amino]-1-(2,2,2- |
| | trifluoroethyl)-1H-indol-2-yl}prop-2-yn-1-yl)amino]benzene-1-sulfonamide |
| 1621. | 3-methoxy-N-methyl-4-[(3-{4-[(1-methylpiperidin-4-yl)amino]-1-(2,2,2-trifluoroethyl)- |
| | 1H-indol-2-yl}prop-2-yn-1-yl)amino]benzene-1-sulfonamide |
| 1622. | 2-[3-({2-methoxy-4-[(4-methylpiperazin-1-yl)sulfonyl]phenyl}amino)prop-1-yn-1-yl]-N- |
| | (1-methylpiperidin-4-yl)-1-(2,2,2-trifluoroethyl)-1H-indol-4-amine |
| 1623. | 5-methanesulfonyl-2-[(3-{4-[(1-methylpiperidin-4-yl)amino]-1-(2,2,2-trifluoroethyl)-1H- |
| | indol-2-yl}prop-2-yn-1-yl)amino]phenol |
| 1624. | 2-{3-[(4-methanesulfonyl-2-methoxyphenyl)amino]prop-1-yn-1-yl}-6-methoxy-N-(1- |
| | methylpiperidin-4-yl)-1-(2,2,2-trifluoroethyl)-1H-indol-4-amine |
| 1625. | 2-{3-[2-(dimethylamino)-4-methanesulfonylphenoxy]prop-1-yn-1-yl}-N-(1- |
| | methylpiperidin-4-yl)-1-(2,2,2-trifluoroethyl)-1H-indol-4-amine |
| 1626. | 2-{5-methanesulfonyl-2-[(3-{4-[(1-methylpiperidin-4-yl)amino]-1-(2,2,2-trifluoroethyl)- |
| | 1H-indol-2-yl}prop-2-yn-1-yl)amino]phenoxy}acetonitrile |
| 1627. | 2-(3-{[2-(fluoromethoxy)-4-methanesulfonylphenyl]amino}prop-1-yn-1-yl)-N-(1- |
| | methylpiperidin-4-yl)-1-(2,2,2-trifluoroethyl)-1H-indol-4-amine |
| 1628. | 2-(3-{[2-methoxy-4-(morpholine-4-carbonyl)phenyl]amino}prop-1-yn-1-yl)-N-(1- |
| | methylpiperidin-4-yl)-1-(2,2,2-trifluoroethyl)-1H-indol-4-amine |
| 1629. | 1-{3-methoxy-4-[(3-{4-[(1-methylpiperidin-4-yl)amino]-1-(2,2,2-trifluoroethyl)-1H- |
| | indol-2-yl}prop-2-yn-1-yl)amino]benzoyl}piperidin-4-ol |
| 1630. | 3-(3-(4-((1-methylpiperidin-4-yl)amino)-1-(2,2,2-trifluoroethyl)-1H-indol-2-yl)prop-2- |
| | yn-1-yl)-6-(methylsulfonyl)benzo[d]oxazol-2(3H)-one |
| 1631. | 2-(3-{[2-methoxy-4-(5-methoxypyridin-3-yl)phenyl]amino}prop-1-yn-1-yl)-N-(1- |
| | methylpiperidin-4-yl)-1-(2,2,2-trifluoroethyl)-1H-indol-4-amine |
| 1632. | 2-{3-[(5-methanesulfonyl-2-methoxyphenyl)amino]prop-1-yn-1-yl}-N-(1- |
| | methylpiperidin-4-yl)-1-(2,2,2-trifluoroethyl)-1H-indol-4-amine |
| 1633. | N-(2-hydroxyethyl)-3-methoxy-4-[(3-{4-[(1-methylpiperidin-4-yl)amino]-1-(2,2,2- |
| | trifluoroethyl)-1H-indol-2-yl}prop-2-yn-1-yl)amino]benzamide |
| 1634. | 3-methoxy-N-(2-methoxyethyl)-4-[(3-{4-[(1-methylpiperidin-4-yl)amino]-1-(2,2,2- |
| | trifluoroethyl)-1H-indol-2-yl}prop-2-yn-1-yl)amino]benzamide |
| 1635. | 3-methoxy-N-(1-methylpiperidin-4-yl)-4-[(3-{4-[(1-methylpiperidin-4-yl)amino]-1- |
| | (2,2,2-trifluoroethyl)-1H-indol-2-yl}prop-2-yn-1-yl)amino]benzamide |
| 1636. | 2-[3-({4-[4-(dimethylamino)piperidine-1-carbonyl]-2-methoxyphenyl}amino)prop-1-yn- |
| | 1-yl]-N-(1-methylpiperidin-4-yl)-1-(2,2,2-trifluoroethyl)-1H-indol-4-amine |
| 1637. | 3-methoxy-4-[(3-{4-[(1-methylpiperidin-4-yl)amino]-1-(2,2,2-trifluoroethyl)-1H-indol-2- |
| | yl}prop-2-yn-1-yl)amino]-N-(oxan-4-yl)benzamide |
| 1638. | 2-(3-{[2-methoxy-4-(4-methylpiperazine-1-carbonyl)phenyl]amino}prop-1-yn-1-yl)-N- |
| | (1-methylpiperidin-4-yl)-1-(2,2,2-trifluoroethyl)-1H-indol-4-amine |
| 1639. | 2-{3-[(2-methoxy-4-{2-oxa-6-azaspiro[3.3]heptane-6-carbonyl}phenyl)amino]prop-1-yn- |
| | 1-yl}-N-(1-methylpiperidin-4-yl)-1-(2,2,2-trifluoroethyl)-1H-indol-4-amine |
| 1640. | 2-(3-{[2-methoxy-4-(pyridin-3-yl)phenyl]amino}prop-1-yn-1-yl)-N-(1-methylpiperidin- |
| | 4-yl)-1-(2,2,2-trifluoroethyl)-1H-indol-4-amine |
| 1641. | 2-(3-{[2-methoxy-4-(pyridin-4-yl)phenyl]amino}prop-1-yn-1-yl)-N-(1-methylpiperidin- |
| | 4-yl)-1-(2,2,2-trifluoroethyl)-1H-indol-4-amine |
| 1642. | N-(3-{4-[(1-methylpiperidin-4-yl)amino]-1-(2,2,2-trifluoroethyl)-1H-indol-2-yl}prop-2- |
| | yn-1-yl)pyridine-3-carboxamide |
| 1643. | 2-(3-{[2-methoxy-4-(1,3-oxazol-2-yl)phenyl]amino}prop-1-yn-1-yl)-N-(1- |
| | methylpiperidin-4-yl)-1-(2,2,2-trifluoroethyl)-1H-indol-4-amine |
| 1644. | 2-{3-[(3-methanesulfonylphenyl)amino]prop-1-yn-1-yl}-N-(1-methylpiperidin-4-yl)-1- |
| | (2,2,2-trifluoroethyl)-1H-indol-4-amine |
| 1645. | N-(1-methylpiperidin-4-yl)-2-[3-({4-[(morpholin-4-yl)methyl]phenyl}amino)prop-1-yn- |
| | 1-yl]-1-(2,2,2-trifluoroethyl)-1H-indol-4-amine |
| 1646. | 2-(3-{[2-methoxy-4-(1,3-thiazol-2-yl)phenyl]amino}prop-1-yn-1-yl)-N-(1- |
| | methylpiperidin-4-yl)-1-(2,2,2-trifluoroethyl)-1H-indol-4-amine |
| 1647. | 2-[3-({2-methoxy-4-[1-(2-methoxyethyl)-1H-pyrazol-4-yl]phenyl}amino)prop-1-yn-1- |
| | yl]-N-(1-methylpiperidin-4-yl)-1-(2,2,2-trifluoroethyl)-1H-indol-4-amine |
| 1648. | 2-{3-[(2-methoxy-4-{7-oxa-2-azaspiro[3.5]nonane-2-carbonyl}phenyl)amino]prop-1-yn- |
| | 1-yl}-N-(1-methylpiperidin-4-yl)-1-(2,2,2-trifluoroethyl)-1H-indol-4-amine |
| 1649. | 2-{3-[(4-chloro-2-methoxyphenyl)amino]prop-1-yn-1-yl}-N-(1-methylpiperidin-4-yl)-1- |
| | (2,2,2-trifluoroethyl)-1H-indol-4-amine |
| 1650. | 2-(3-(((3S,4R)-3-methoxytetrahydro-2H-pyran-4-yl)amino)prop-1-yn-1-yl)-N-(1- |
| | methylpiperidin-4-yl)-1-(2,2,2-trifluoroethyl)-1H-indol-4-amine |
| 1651. | 2-fluoro-5-methoxy-4-[(3-{4-[(1-methylpiperidin-4-yl)amino]-1-(2,2,2-trifluoroethyl)- |
| | 1H-indol-2-yl}prop-2-yn-1-yl)amino]benzamide |
| 1652. | 2-(3-{[2-methoxy-4-(trifluoromethyl)phenyl]amino}prop-1-yn-1-yl)-N-(1- |
| | methylpiperidin-4-yl)-1-(2,2,2-trifluoroethyl)-1H-indol-4-amine |
| 1653. | 2-fluoro-5-methoxy-N-methyl-4-[(3-{4-[(1-methylpiperidin-4-yl)amino]-1-(2,2,2- |
| | trifluoroethyl)-1H-indol-2-yl}prop-2-yn-1-yl)amino]benzamide |
| 1654. | 2-{3-[(4-methanesulfonyl-2-methoxyphenyl)amino]prop-1-yn-1-yl}-N-(1- |
| | methylpiperidin-4-yl)-1-[(oxiran-2-yl)methyl]-1H-indol-4-amine |
| 1655. | 2-(3-((2-ethoxy-4-(methylsulfonyl)phenyl)amino)prop-1-yn-1-yl)-N-(1-methylpiperidin- |
| | 4-yl)-1-(2,2,2-trifluoroethyl)-1H-indol-4-amine |
| 1656. | 2-{3-[(2-fluoro-4-methanesulfonyl-6-methoxyphenyl)amino]prop-1-yn-1-yl}-N-(1- |
| | methylpiperidin-4-yl)-1-(2,2,2-trifluoroethyl)-1H-indol-4-amine |
| 1657. | 2-{3-[(4-methanesulfonyl-2-methoxyphenyl)amino]prop-1-yn-1-yl}-3-methyl-N-(1- |
| | methylpiperidin-4-yl)-1-(2,2,2-trifluoroethyl)-1H-indol-4-amine |
| 1658. | 2-(3-(((3R,4R)-3-methoxytetrahydro-2H-pyran-4-yl)amino)prop-1-yn-1-yl)-N-(1- |
| | methylpiperidin-4-yl)-1-(2,2,2-trifluoroethyl)-1H-indol-4-amine |
| 1659. | 2-{3-[(5-fluoro-4-methanesulfonyl-2-methoxyphenyl)amino]prop-1-yn-1-yl}-N-(1- |
| | methylpiperidin-4-yl)-1-(2,2,2-trifluoroethyl)-1H-indol-4-amine |
| 1660. | 2-{3-[(5-methanesulfonylthiophen-2-yl)amino]prop-1-yn-1-yl}-N-(1-methylpiperidin-4- |
| | yl)-1-(2,2,2-trifluoroethyl)-1H-indol-4-amine |
| 1661. | N-methyl-5-[(3-{4-[(1-methylpiperidin-4-yl)amino]-1-(2,2,2-trifluoroethyl)-1H-indol-2- |
| | yl}prop-2-yn-1-yl)amino]thiophene-2-carboxamide |
| 1662. | N,N-dimethyl-5-[(3-{4-[(1-methylpiperidin-4-yl)amino]-1-(2,2,2-trifluoroethyl)-1H- |
| | indol-2-yl}prop-2-yn-1-yl)amino]thiophene-2-carboxamide |
| 1663. | 5-[(3-{4-[(1-methylpiperidin-4-yl)amino]-1-(2,2,2-trifluoroethyl)-1H-indol-2-yl}prop-2- |
| | yn-1-yl)amino]thiophene-2-carboxylic acid |
| 1664. | 2-(3-((4-methoxypyridin-3-yl)amino)prop-1-yn-1-yl)-N-(1-methylpiperidin-4-yl)-1- |
| | (2,2,2-trifluoroethyl)-1H-indol-4-amine |
| 1665. | 2-(2-((3-(4-((1-methylpiperidin-4-yl)amino)-1-(2,2,2-trifluoroethyl)-1H-indol-2-yl)prop- |
| | 2-yn-1-yl)amino)-5-(methylsulfonyl)phenoxy)acetamide |
| 1666. | N-(2-hydroxyethyl)-3-methoxy-4-((3-(4-((1-methylpiperidin-4-yl)amino)-1-(2,2,2- |
| | trifluoroethyl)-1H-indol-2-yl)prop-2-yn-1-yl)amino)benzenesulfonamide |
| 1667. | 4-((2-(3-((2-methoxy-4-(methylsulfonyl)phenyl)amino)prop-1-yn-1-yl)-1-(2,2,2- |
| | trifluoroethyl)-1H-indol-4-yl)amino)-1-methylpiperidin-2-one |
| 1668. | 2-{3-[(4-methanesulfonyl-2-methoxyphenyl)amino]prop-1-yn-1-yl}-N-[(3R,4S)-3- |
| | methoxy-1-methylpiperidin-4-yl]-1-(2,2,2-trifluoroethyl)-1H-indol-4-amine |
| 1669. | 2-{3-[(4-methanesulfonyl-2-methoxyphenyl)amino]prop-1-yn-1-yl}-N-[(3S,4R)-3- |
| | methoxy-1-methylpiperidin-4-yl]-1-(2,2,2-trifluoroethyl)-1H-indol-4-amine |
| 1670. | 2-{3-[(4-methanesulfonyl-2-methoxyphenyl)amino]prop-1-yn-1-yl}-N-[(3S,4R)-3- |
| | methoxy-1-methylpiperidin-4-yl]-N-methyl-1-(2,2,2-trifluoroethyl)-1H-indol-4-amine |
| 1671. | N-(1-ethylpiperidin-4-yl)-2-(3-{[2-(fluoromethoxy)-4- |
| | methanesulfonylphenyl]amino}prop-1-yn-1-yl)-1-(2,2,2-trifluoroethyl)-1H-indol-4-amine |
| 1672. | 4-[(3-{4-[(1-ethylpiperidin-4-yl)amino]-1-(2,2,2-trifluoroethyl)-1H-indol-2-yl}prop-2-yn- |
| | 1-yl)amino]-3-methoxy-N-methylbenzamide |
| 1673. | 2-{2-[(3-{4-[(1-ethylpiperidin-4-yl)amino]-1-(2,2,2-trifluoroethyl)-1H-indol-2-yl}prop-2- |
| | yn-1-yl)amino]-5-methanesulfonylphenoxy}acetonitrile |
| 1674. | N-(1-ethylpiperidin-4-yl)-2-{3-[(4-methanesulfonyl-2-methoxyphenyl)amino]prop-1-yn- |
| | 1-yl}-1-(2,2,2-trifluoroethyl)-1H-indol-4-amine |
| 1675. | 4-[(3-{4-[(1-ethylpiperidin-4-yl)amino]-1-(2,2,2-trifluoroethyl)-1H-indol-2-yl}prop-2-yn- |
| | 1-yl)amino]-3-methoxybenzene-1-sulfonamide |
| 1676. | 3-methoxy-N-methyl-4-{[3-(4-{[1-(propan-2-yl)piperidin-4-yl]amino}-1-(2,2,2- |
| | trifluoroethyl)-1H-indol-2-yl)prop-2-yn-1-yl]amino}benzamide |
| 1677. | 2-(5-methanesulfonyl-2-{[3-(4-{[1-(propan-2-yl)piperidin-4-yl]amino}-1-(2,2,2- |
| | trifluoroethyl)-1H-indol-2-yl)prop-2-yn-1-yl]amino}phenoxy)acetonitrile |
| 1678. | 2-(3-{[2-(fluoromethoxy)-4-methanesulfonylphenyl]amino}prop-1-yn-1-yl)-N-[1- |
| | (propan-2-yl)piperidin-4-yl]-1-(2,2,2-trifluoroethyl)-1H-indol-4-amine |
| 1679. | 2-{3-[(4-methanesulfonyl-2-methoxyphenyl)amino]prop-1-yn-1-yl}-N-[1-(propan-2- |
| | yl)piperidin-4-yl]-1-(2,2,2-trifluoroethyl)-1H-indol-4-amine |
| 1680. | 3-methoxy-4-{[3-(4-{[1-(propan-2-yl)piperidin-4-yl]amino}-1-(2,2,2-trifluoroethyl)-1H- |
| | indol-2-yl)prop-2-yn-1-yl]amino}benzene-1-sulfonamide |
| 1681. | 2-[2-(2-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}ethoxy)ethoxy]ethan-1-ol |
| 1682. | 4-({4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}methyl)-1,3-dioxolan-2-one |
| 1683. | 3-methoxy-4-({3-[4-({1-[(2-oxo-1,3-dioxolan-4-yl)methyl]piperidin-4-yl}amino)-1- |
| | (2,2,2-trifluoroethyl)-1H-indol-2-yl]prop-2-yn-1-yl}amino)benzene-1-sulfonamide |
| 1684. | 4-{[3-(4-{[1-(2,3-dihydroxypropyl)piperidin-4-yl]amino}-1-(2,2,2-trifluoroethyl)-1H- |
| | indol-2-yl)prop-2-yn-1-yl]amino}-3-methoxy-N-(1,2-oxazol-3-yl)benzene-1-sulfonamide |
| 1685. | 4-((3-(4-((1-(2,3-dihydroxypropyl)piperidin-4-yl)amino)-1-(2,2,2-trifluoroethyl)-1H- |
| | indol-2-yl)prop-2-yn-1-yl)amino)-3-methoxybenzenesulfonamide |
| 1686. | 4-({3-[4-({1-[(2S)-2,3-dihydroxypropyl]piperidin-4-yl}amino)-1-(2,2,2-trifluoroethyl)- |
| | 1H-indol-2-yl]prop-2-yn-1-yl}amino)-3-methoxybenzene-1-sulfonamide |
| 1687. | 4-({3-[4-({1-[(2R)-2,3-dihydroxypropyl]piperidin-4-yl}amino)-1-(2,2,2-trifluoroethyl)- |
| | 1H-indol-2-yl]prop-2-yn-1-yl}amino)-3-methoxybenzene-1-sulfonamide |
| 1688. | 4-{[3-(4-{[1-(2,3-dihydroxypropyl)piperidin-4-yl]amino}-1-(2,2,2-trifluoroethyl)-1H- |
| | indol-2-yl)prop-2-yn-1-yl]amino}-3-methoxy-N-(5-methyl-1,2-oxazol-3-yl)benzene-1- |
| | sulfonamide |
| 1689. | 4-{[3-(4-{[1-(2,3-dihydroxypropyl)piperidin-4-yl]amino}-1-(2,2,2-trifluoroethyl)-1H- |
| | indol-2-yl)prop-2-yn-1-yl]amino}-3-methoxy-N-(oxan-4-yl)benzene-1-sulfonamide |
| 1690. | 4-{[3-(4-{[1-(2,3-dihydroxypropyl)piperidin-4-yl]amino}-1-(2,2,2-trifluoroethyl)-1H- |
| | indol-2-yl)prop-2-yn-1-yl]amino}-N-(2-hydroxyethyl)-3-methoxy-N-methylbenzene-1- |
| | sulfonamide |
| 1691. | 2-(5-{[3-(4-{[1-(2,3-dihydroxypropyl)piperidin-4-yl]amino}-1-(2,2,2-trifluoroethyl)-1H- |
| | indol-2-yl)prop-2-yn-1-yl]amino}-4-methoxypyridin-2-yl)-2-methylpropanenitrile |
| 1692. | N-(2-hydroxyethyl)-3-methoxy-N-methyl-4-{[3-(4-{[1-(oxan-4-yl)piperidin-4-yl]amino}- |
| | 1-(2,2,2-trifluoroethyl)-1H-indol-2-yl)prop-2-yn-1-yl]amino}benzene-1-sulfonamide |
| 1693. | 3-(4-((2-(3-((2-methoxy-4-(methylsulfonyl)phenyl)amino)prop-1-yn-1-yl)-1-(2,2,2- |
| | trifluoroethyl)-1H-indol-4-yl)amino)piperidin-1-yl)propane-1,2-diol |
| 1694. | (2R)-3-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}propane-1,2-diol |
| 1695. | (2S)-3-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}propane-1,2-diol |
| 1696. | 3-[4-({2-[3-({2-methoxy-4-[(4-methylpiperazin-1-yl)sulfonyl]phenyl}amino)prop-1-yn-1- |
| | yl]-1-(2,2,2-trifluoroethyl)-1H-indol-4-yl}amino)piperidin-1-yl]propane-1,2-diol |
| 1697. | 4-((3-(4-((1-(2,3-dihydroxypropyl)piperidin-4-yl)amino)-1-(2,2,2-trifluoroethyl)-1H- |
| | indol-2-yl)prop-2-yn-1-yl)amino)-3-methoxybenzoic acid |
| 1698. | methyl 4-((3-(4-((1-(2,3-dihydroxypropyl)piperidin-4-yl)amino)-1-(2,2,2-trifluoroethyl)- |
| | 1H-indol-2-yl)prop-2-yn-1-yl)amino)-3-methoxybenzoate |
| 1699. | 3-methoxy-4-[(3-{4-[(1-{[(4R)-2-oxo-1,3-dioxolan-4-yl]methyl}piperidin-4-yl)amino]-1- |
| | (2,2,2-trifluoroethyl)-1H-indol-2-yl}prop-2-yn-1-yl)amino]benzene-1-sulfonamide |
| 1700. | (4R)-4-({4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}methyl)-1,3-dioxolan-2-one |
| 1701. | 4-{[3-(4-{[1-(2,3-dihydroxypropyl)piperidin-4-yl]amino}-1-(2,2,2-trifluoroethyl)-1H- |
| | indol-2-yl)prop-2-yn-1-yl]amino}-3-methoxy-N,N-dimethylbenzene-1-sulfonamide |
| 1702. | 2-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}EtOAc |
| 1703. | N-(1-{[(4R)-2,2-dimethyl-1,3-dioxolan-4-yl]methyl}piperidin-4-yl)-2-{3-[(4- |
| | methanesulfonyl-2-methoxyphenyl)amino]prop-1-yn-1-yl}-1-(2,2,2-trifluoroethyl)-1H- |
| | indol-4-amine |
| 1704. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}propan-2-yl acetate |
| 1705. | 4-[(3-{4-[(1-{[(4R)-2,2-dimethyl-1,3-dioxolan-4-yl]methyl}piperidin-4-yl)amino]-1- |
| | (2,2,2-trifluoroethyl)-1H-indol-2-yl}prop-2-yn-1-yl)amino]-3-methoxybenzene-1- |
| | sulfonamide |
| 1706. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-3-methoxypropan-2-ol |
| 1707. | 1-(4-((2-(3-((4-(ethylsulfonyl)-2-methoxyphenyl)amino)prop-1-yn-1-yl)-1-(2,2,2- |
| | trifluoroethyl)-1H-indol-4-yl)amino)piperidin-1-yl)-3-methoxypropan-2-ol |
| 1708. | 1-ethoxy-3-(4-((2-(3-((2-methoxy-4-(methylsulfonyl)phenyl)amino)prop-1-yn-1-yl)-1- |
| | (2,2,2-trifluoroethyl)-1H-indol-4-yl)amino)piperidin-1-yl)propan-2-ol |
| 1709. | 4-{[3-(4-{[1-(2,3-dihydroxypropyl)piperidin-4-yl]amino}-1-(2,2,2-trifluoroethyl)-1H- |
| | indol-2-yl)prop-2-yn-1-yl]amino}-3-methoxy-N-(2-methoxyethyl)-N-methylbenzene-1- |
| | sulfonamide |
| 1710. | 1-(acetyloxy)-3-{4-[(2-{3-[(2-methoxy-4-sulfamoylphenyl)amino]prop-1-yn-1-yl}-1- |
| | (2,2,2-trifluoroethyl)-1H-indol-4-yl)amino]piperidin-1-yl}propan-2-yl acetate |
| 1711. | 4-{[3-(4-{[1-(2-hydroxy-3-methoxypropyl)piperidin-4-yl]amino}-1-(2,2,2-trifluoroethyl)- |
| | 1H-indol-2-yl)prop-2-yn-1-yl]amino}-3-methoxybenzene-1-sulfonamide |
| 1712. | 3-methoxy-4-{[3-(4-{[1-(2-methoxyacetyl)piperidin-4-yl]amino}-1-(2,2,2-trifluoroethyl)- |
| | 1H-indol-2-yl)prop-2-yn-1-yl]amino}benzene-1-sulfonamide |
| 1713. | 1-(4-(N-(2-(3-((2-methoxy-4-(methylsulfonyl)phenyl)amino)prop-1-yn-1-yl)-1-(2,2,2- |
| | trifluoroethyl)-1H-indol-4-yl)acetamido)piperidin-1-yl)propan-2-yl acetate |
| 1714. | 1-[4-(4-{[3-(4-{[1-(2,3-dihydroxypropyl)piperidin-4-yl]amino}-1-(2,2,2-trifluoroethyl)- |
| | 1H-indol-2-yl)prop-2-yn-1-yl]amino}-3-methoxybenzenesulfonyl)piperazin-1-yl]ethan-1-one |
| 1715. | (4S)-4-({4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}methyl)-1,3-dioxolan-2-one |
| 1716. | 1-(acetyloxy)-3-{4-[(2-{3-[(4-methanesulfonyl-2-methoxyphenyl)amino]prop-1-yn-1-yl}- |
| | 1-(2,2,2-trifluoroethyl)-1H-indol-4-yl)amino]piperidin-1-yl}propan-2-yl acetate |
| 1717. | N-[1-(2,3-dimethoxypropyl)piperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1718. | 4-{[3-(4-{[1-(2,3-dimethoxypropyl)piperidin-4-yl]amino}-1-(2,2,2-trifluoroethyl)-1H- |
| | indol-2-yl)prop-2-yn-1-yl]amino}-3-methoxybenzene-1-sulfonamide |
| 1719. | 3-(4-{[2-(3-{[2-methoxy-4-(morpholine-4-sulfonyl)phenyl]amino}prop-1-yn-1-yl)-1- |
| | (2,2,2-trifluoroethyl)-1H-indol-4-yl]amino}piperidin-1-yl)propane-1,2-diol |
| 1720. | 4-({3-[4-({1-[(2R)-2,3-dihydroxypropyl]piperidin-4-yl}amino)-1-(2,2,2-trifluoroethyl)- |
| | 1H-indol-2-yl]prop-2-yn-1-yl}amino)-3-methoxybenzamide |
| 1721. | 2-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}ethyl 2-methylpropanoate |
| 1722. | 2-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}ethyl propanoate |
| 1723. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-3-(propanoyloxy)propan-2-yl |
| | propanoate |
| 1724. | 1-{4-[(2-{3-[(2-methoxy-4-sulfamoylphenyl)amino]prop-1-yn-1-yl}-1-(2,2,2- |
| | trifluoroethyl)-1H-indol-4-yl)amino]piperidin-1-yl}-3-(propanoyloxy)propan-2-yl |
| | propanoate |
| 1725. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-3-[(2-methylpropanoyl)oxy]propan- |
| | 2-yl 2-methylpropanoate |
| 1726. | 1-{4-[(2-{3-[(2-methoxy-4-sulfamoylphenyl)amino]prop-1-yn-1-yl}-1-(2,2,2- |
| | trifluoroethyl)-1H-indol-4-yl)amino]piperidin-1-yl}-3-[(2-methylpropanoyl)oxy]propan- |
| | 2-yl 2-methylpropanoate |
| 1727. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}propan-2-yl propanoate |
| 1728. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}propan-2-yl 2-methylpropanoate |
| 1729. | 2-hydroxy-3-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}propyl 2-methylpropanoate |
| 1730. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-3-methoxypropan-2-yl acetate |
| 1731. | 2-hydroxy-3-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}propyl propanoate |
| 1732. | N,N-bis(2-hydroxyethyl)-4-{[3-(4-{[1-(2-hydroxypropyl)piperidin-4-yl]amino}-1-(2,2,2- |
| | trifluoroethyl)-1H-indol-2-yl)prop-2-yn-1-yl]amino}-3-methoxybenzene-1-sulfonamide |
| 1733. | 4-{[3-(4-{[1-(2-hydroxypropyl)piperidin-4-yl]amino}-1-(2,2,2-trifluoroethyl)-1H-indol-2- |
| | yl)prop-2-yn-1-yl]amino}-3-methoxy-N-methylbenzamide |
| 1734. | (S)-4-((3-(4-((1-(2-hydroxy-3-methoxypropyl)piperidin-4-yl)amino)-1-(2,2,2- |
| | trifluoroethyl)-1H-indol-2-yl)prop-2-yn-1-yl)amino)-3-methoxy-N-methylbenzamide |
| 1735. | 1-{4-[(2-{3-[(2-methoxy-4-sulfamoylphenyl)amino]prop-1-yn-1-yl}-1-(2,2,2- |
| | trifluoroethyl)-1H-indol-4-yl)amino]piperidin-1-yl}propan-2-yl 2-methylpropanoate |
| 1736. | 1-{4-[(2-{3-[(2-methoxy-4-sulfamoylphenyl)amino]prop-1-yn-1-yl}-1-(2,2,2- |
| | trifluoroethyl)-1H-indol-4-yl)amino]piperidin-1-yl}propan-2-yl acetate |
| 1737. | 2-{4-[(2-{3-[(2-methoxy-4-sulfamoylphenyl)amino]prop-1-yn-1-yl}-1-(2,2,2- |
| | trifluoroethyl)-1H-indol-4-yl)amino]piperidin-1-yl}ethyl 2-methylpropanoate |
| 1738. | 4-{[3-(4-{[1-(2-hydroxy-3-methoxypropyl)piperidin-4-yl]amino}-1-(2,2,2-trifluoroethyl)- |
| | 1H-indol-2-yl)prop-2-yn-1-yl]amino}-3-methoxy-N-methylbenzamide |
| 1739. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-3-methoxypropan-2-yl propanoate |
| 1740. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-3-methoxypropan-2-yl 2- |
| | methylpropanoate |
| 1741. | 1-methoxy-3-{4-[(2-{3-[(2-methoxy-4-sulfamoylphenyl)amino]prop-1-yn-1-yl}-1-(2,2,2- |
| | trifluoroethyl)-1H-indol-4-yl)amino]piperidin-1-yl}propan-2-yl 2-methylpropanoate |
| 1742. | 1-{4-[(2-{3-[(2-methoxy-4-sulfamoylphenyl)amino]prop-1-yn-1-yl}-1-(2,2,2- |
| | trifluoroethyl)-1H-indol-4-yl)amino]piperidin-1-yl}propan-2-yl propanoate |
| 1743. | N-(4-{[3-(4-{[1-(2-hydroxy-3-methoxypropyl)piperidin-4-yl]amino}-1-(2,2,2- |
| | trifluoroethyl)-1H-indol-2-yl)prop-2-yn-1-yl]amino}-3- |
| | methoxybenzenesulfonyl)acetamide |
| 1744. | N-(4-{[3-(4-{[1-(2-hydroxy-3-methoxypropyl)piperidin-4-yl]amino}-1-(2,2,2- |
| | trifluoroethyl)-1H-indol-2-yl)prop-2-yn-1-yl]amino}-3- |
| | methoxybenzenesulfonyl)propanamide |
| 1745. | 2-{4-[(2-{3-[(2-methoxy-4-sulfamoylphenyl)amino]prop-1-yn-1-yl}-1-(2,2,2- |
| | trifluoroethyl)-1H-indol-4-yl)amino]piperidin-1-yl}ethyl propanoate |
| 1746. | 4-{[3-(4-{[1-(2-hydroxy-3-methoxypropyl)piperidin-4-yl]amino}-1-(2,2,2-trifluoroethyl)- |
| | 1H-indol-2-yl)prop-2-yn-1-yl]amino}-3-methoxy-N-methylbenzene-1-sulfonamide |
| 1747. | 1-methoxy-3-{4-[(2-{3-[(2-methoxy-4-sulfamoylphenyl)amino]prop-1-yn-1-yl}-1-(2,2,2- |
| | trifluoroethyl)-1H-indol-4-yl)amino]piperidin-1-yl}propan-2-yl propanoate |
| 1748. | (2R)-1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-3-methoxypropan-2-ol |
| 1749. | (2S)-1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-3-methoxypropan-2-ol |
| 1750. | 3-methoxy-4-{[3-(4-{[(1S,4S)-4-(dimethylamino)cyclohexyl]amino}-1-(2,2,2- |
| | trifluoroethyl)-1H-indol-2-yl)prop-2-yn-1-yl]amino}benzamide |
| 1751. | 3-methoxy-4-((3-(4-((1-(2-methoxyethyl)piperidin-4-yl)amino)-1-(2,2,2-trifluoroethyl)- |
| | 1H-indol-2-yl)prop-2-yn-1-yl)amino)benzenesulfonamide |
| 1752. | 2-(3-((2-methoxy-4-(methylsulfonyl)phenyl)amino)prop-1-yn-1-yl)-N-(1-(2- |
| | methoxyethyl)piperidin-4-yl)-1-(2,2,2-trifluoroethyl)-1H-indol-4-amine |
| 1753. | 3-methoxy-4-{[3-(4-{[1-(2-methoxyethyl)piperidin-4-yl]amino}-1-(2,2,2-trifluoroethyl)- |
| | 1H-indol-2-yl)prop-2-yn-1-yl]amino}-N-methylbenzamide |
| 1754. | 3-methoxy-4-{[3-(4-{[1-(2-methoxyethyl)piperidin-4-yl]amino}-1-(2,2,2-trifluoroethyl)- |
| | 1H-indol-2-yl)prop-2-yn-1-yl]amino}-N-methylbenzene-1-sulfonamide |
| 1755. | 2-{4-[(2-{3-[(2-methoxy-4-sulfamoylphenyl)amino]prop-1-yn-1-yl}-1-(2,2,2- |
| | trifluoroethyl)-1H-indol-4-yl)amino]piperidin-1-yl}EtOAc |
| 1756. | 1-methoxy-3-{4-[(2-{3-[(2-methoxy-4-sulfamoylphenyl)amino]prop-1-yn-1-yl}-1-(2,2,2- |
| | trifluoroethyl)-1H-indol-4-yl)amino]piperidin-1-yl}propan-2-yl acetate |
| 1757. | 4-{[3-(4-{[1-(2-hydroxy-3-methoxypropyl)piperidin-4-yl]amino}-1-(2,2,2-trifluoroethyl)- |
| | 1H-indol-2-yl)prop-2-yn-1-yl]amino}-3-methoxybenzamide |
| 1758. | 2-{[3-(4-{[1-(2-hydroxy-3-methoxypropyl)piperidin-4-yl]amino}-1-(2,2,2-trifluoroethyl)- |
| | 1H-indol-2-yl)prop-2-yn-1-yl]amino}-5-methanesulfonylphenol |
| 1759. | 4-{[3-(4-{[1-(2-hydroxy-3-methoxypropyl)piperidin-4-yl]amino}-1-(2,2,2-trifluoroethyl)- |
| | 1H-indol-2-yl)prop-2-yn-1-yl]amino}-3-methoxy-N,N-dimethylbenzene-1-sulfonamide |
| 1760. | 4-({3-[4-({1-[(2R)-3-hydroxy-2-methoxypropyl]piperidin-4-yl}amino)-1-(2,2,2- |
| | trifluoroethyl)-1H-indol-2-yl]prop-2-yn-1-yl}amino)-3-methoxybenzene-1-sulfonamide |
| 1761. | N-(4-{[3-(4-{[1-(2-hydroxy-3-methoxypropyl)piperidin-4-yl]amino}-1-(2,2,2- |
| | trifluoroethyl)-1H-indol-2-yl)prop-2-yn-1-yl]amino}-3-methoxybenzenesulfonyl)-N- |
| | methylpropanamide |
| 1762. | 1-(4-{[2-(3-{[2-(2-fluoroethoxy)-4-methanesulfonylphenyl]amino}prop-1-yn-1-yl)-1- |
| | (2,2,2-trifluoroethyl)-1H-indol-4-yl]amino}piperidin-1-yl)-3-methoxypropan-2-ol |
| 1763. | 1-(4-((2-(3-((2-methoxy-4-(methylsulfonyl)phenyl)amino)prop-1-yn-1-yl)-1-(2,2,2- |
| | trifluoroethyl)-1H-indol-4-yl)amino)piperidin-1-yl)tetradecan-1-one |
| 1764. | 1-methoxy-3-(4-{[2-(3-{[2-methoxy-4-(propanamidosulfonyl)phenyl]amino}prop-1-yn-1- |
| | yl)-1-(2,2,2-trifluoroethyl)-1H-indol-4-yl]amino}piperidin-1-yl)propan-2-yl propanoate |
| 1765. | 1-(4-{[2-(3-{[2-methoxy-4-(propanamidosulfonyl)phenyl]amino}prop-1-yn-1-yl)-1- |
| | (2,2,2-trifluoroethyl)-1H-indol-4-yl]amino}piperidin-1-yl)-3-(propanoyloxy)propan-2-yl |
| | propanoate |
| 1766. | (2R)-3-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-2-methoxypropan-1-ol |
| 1767. | (2S)-3-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-2-methoxypropan-1-ol |
| 1768. | 1-{4-[(2-{3-[(2-ethoxy-4-methanesulfonylphenyl)amino]prop-1-yn-1-yl}-1-(2,2,2- |
| | trifluoroethyl)-1H-indol-4-yl)amino]piperidin-1-yl}-3-methoxypropan-2-ol |
| 1769. | 4-({3-[4-({1-[(2S)-3-hydroxy-2-methoxypropyl]piperidin-4-yl}amino)-1-(2,2,2- |
| | trifluoroethyl)-1H-indol-2-yl]prop-2-yn-1-yl}amino)-3-methoxybenzene-1-sulfonamide |
| 1770. | 4-{[3-(4-{[1-(2-hydroxy-3-methoxypropyl)piperidin-4-yl]amino}-1-(2,2,2-trifluoroethyl)- |
| | 1H-indol-2-yl)prop-2-yn-1-yl]amino}-3-methoxy-N-(propan-2-yl)benzamide |
| 1771. | 3-hydroxy-4-{[3-(4-{[1-(2-hydroxy-3-methoxypropyl)piperidin-4-yl]amino}-1-(2,2,2- |
| | trifluoroethyl)-1H-indol-2-yl)prop-2-yn-1-yl]amino}benzene-1-sulfonamide |
| 1772. | (2R)-1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-3-methoxypropan-2-yl 2- |
| | methylpropanoate |
| 1773. | N-(3-(4-((1-(2-hydroxy-3-methoxypropyl)piperidin-4-yl)amino)-1-(2,2,2-trifluoroethyl)- |
| | 1H-indol-2-yl)prop-2-yn-1-yl)-N-(2-hydroxy-4-(N- |
| | propionylsulfamoyl)phenyl)propionamide |
| 1774. | 1-(4-{[2-(3-{[4-methanesulfonyl-2-(2-methoxyethoxy)phenyl]amino}prop-1-yn-1-yl)-1- |
| | (2,2,2-trifluoroethyl)-1H-indol-4-yl]amino}piperidin-1-yl)-3-methoxypropan-2-ol |
| 1775. | (2S)-1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-3-methoxypropan-2-yl 2- |
| | methylpropanoate |
| 1776. | N-(3-(4-((1-(2-hydroxy-3-methoxypropyl)piperidin-4-yl)amino)-1-(2,2,2-trifluoroethyl)- |
| | 1H-indol-2-yl)prop-2-yn-1-yl)-N-(2-hydroxy-4-(methylsulfonyl)phenyl)acetamide |
| 1777. | N-(3-(4-((1-(2-hydroxy-3-methoxypropyl)piperidin-4-yl)amino)-1-(2,2,2-trifluoroethyl)- |
| | 1H-indol-2-yl)prop-2-yn-1-yl)-N-(2-hydroxy-4-(methylsulfonyl)phenyl)propionamide |
| 1778. | N-(3-(4-((1-(2-hydroxy-3-methoxypropyl)piperidin-4-yl)amino)-1-(2,2,2-trifluoroethyl)- |
| | 1H-indol-2-yl)prop-2-yn-1-yl)-N-(2-hydroxy-4-(methylsulfonyl)phenyl)isobutyramide |
| 1779. | 2-(2-{[3-(4-{[1-(2-hydroxy-3-methoxypropyl)piperidin-4-yl]amino}-1-(2,2,2- |
| | trifluoroethyl)-1H-indol-2-yl)prop-2-yn-1-yl]amino}-5- |
| | methanesulfonylphenoxy)acetonitrile |
| 1780. | 1-{4-[(2-{3-[(4-methanesulfonyl-2-methoxyphenyl)amino]prop-1-yn-1-yl}-5-methoxy-1- |
| | (2,2,2-trifluoroethyl)-1H-indol-4-yl)amino]piperidin-1-yl}-3-methoxypropan-2-ol |
| 1781. | 1-(4-{[2-(3-{[2-(2-hydroxyethoxy)-4-methanesulfonylphenyl]amino}prop-1-yn-1-yl)-1- |
| | (2,2,2-trifluoroethyl)-1H-indol-4-yl]amino}piperidin-1-yl)-3-methoxypropan-2-ol |
| 1782. | 1-(4-{[2-(3-{[4-methanesulfonyl-2-(2,2,2-trifluoroethoxy)phenyl]amino}prop-1-yn-1-yl)- |
| | 1-(2,2,2-trifluoroethyl)-1H-indol-4-yl]amino}piperidin-1-yl)-3-methoxypropan-2-ol |
| 1783. | 1-[4-({2-[3-(2-amino-4-methanesulfonylphenoxy)prop-1-yn-1-yl]-1-(2,2,2-trifluoroethyl)- |
| | 1H-indol-4-yl}amino)piperidin-1-yl]-3-methoxypropan-2-ol |
| 1784. | 4-({3-[4-({1-[(2R)-2-hydroxy-3-methoxypropyl]piperidin-4-yl}amino)-1-(2,2,2- |
| | trifluoroethyl)-1H-indol-2-yl]prop-2-yn-1-yl}amino)-3-methoxy-N-methylbenzamide |
| 1785. | 4-({3-[4-({1-[(2S)-2-hydroxy-3-methoxypropyl]piperidin-4-yl}amino)-1-(2,2,2- |
| | trifluoroethyl)-1H-indol-2-yl]prop-2-yn-1-yl}amino)-3-methoxy-N-methylbenzamide |
| 1786. | 4-({3-[4-({1-[(2R)-3-hydroxy-2-methoxypropyl]piperidin-4-yl}amino)-1-(2,2,2- |
| | trifluoroethyl)-1H-indol-2-yl]prop-2-yn-1-yl}amino)-3-methoxy-N-methylbenzamide |
| 1787. | 4-({3-[4-({1-[(2S)-3-hydroxy-2-methoxypropyl]piperidin-4-yl}amino)-1-(2,2,2- |
| | trifluoroethyl)-1H-indol-2-yl]prop-2-yn-1-yl}amino)-3-methoxy-N-methylbenzamide |
| 1788. | 1-{4-[(2-{3-[4-methanesulfonyl-2-(methylamino)phenoxy]prop-1-yn-1-yl}-1-(2,2,2- |
| | trifluoroethyl)-1H-indol-4-yl)amino]piperidin-1-yl}-3-methoxypropan-2-ol |
| 1789. | 3-(2-fluoroethoxy)-4-{[3-(4-{[1-(2-hydroxy-3-methoxypropyl)piperidin-4-yl]amino}-1- |
| | (2,2,2-trifluoroethyl)-1H-indol-2-yl)prop-2-yn-1-yl]amino}-N-methylbenzamide |
| 1790. | 4-{[3-(4-{[1-(2-hydroxy-3-methoxypropyl)piperidin-4-yl]amino}-1-(2,2,2-trifluoroethyl)- |
| | 1H-indol-2-yl)prop-2-yn-1-yl]amino}-3-(2-methoxyethoxy)-N-methylbenzamide |
| 1791. | 3-(cyanomethoxy)-4-{[3-(4-{[1-(2-hydroxy-3-methoxypropyl)piperidin-4-yl]amino}-1- |
| | (2,2,2-trifluoroethyl)-1H-indol-2-yl)prop-2-yn-1-yl]amino}-N-methylbenzamide |
| 1792. | N-ethyl-4-{[3-(4-{[1-(2-hydroxy-3-methoxypropyl)piperidin-4-yl]amino}-1-(2,2,2- |
| | trifluoroethyl)-1H-indol-2-yl)prop-2-yn-1-yl]amino}-3-methoxybenzamide |
| 1793. | 1-(4-{[2-(3-{[2-methoxy-4-(methylcarbamoyl)phenyl]amino}prop-1-yn-1-yl)-1-(2,2,2- |
| | trifluoroethyl)-1H-indol-4-yl]amino}piperidin-1-yl)-3-(propanoyloxy)propan-2-yl |
| | propanoate |
| 1794. | 1-methoxy-3-(4-{[2-(3-{[2-methoxy-4-(methylcarbamoyl)phenyl]amino}prop-1-yn-1-yl)- |
| | 1-(2,2,2-trifluoroethyl)-1H-indol-4-yl]amino}piperidin-1-yl)propan-2-yl propanoate |
| 1795. | 4-{[3-(4-{[1-(2,3-dihydroxypropyl)piperidin-4-yl]amino}-1-(2,2,2-trifluoroethyl)-1H- |
| | indol-2-yl)prop-2-yn-1-yl]amino}-3-methoxy-N-methylbenzamide |
| 1796. | 3-(2-cyanoethoxy)-4-{[3-(4-{[1-(2-hydroxy-3-methoxypropyl)piperidin-4-yl]amino}-1- |
| | (2,2,2-trifluoroethyl)-1H-indol-2-yl)prop-2-yn-1-yl]amino}-N-methylbenzamide |
| 1797. | 1-ethoxy-3-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}propan-2-ol |
| 1798. | 2-(2-{[3-(4-{[1-(2-hydroxypropyl)piperidin-4-yl]amino}-1-(2,2,2-trifluoroethyl)-1H- |
| | indol-2-yl)prop-2-yn-1-yl]amino}-5-methanesulfonylphenoxy)acetonitrile |
| 1799. | 1-(4-{[2-(3-{[4-(ethanesulfonyl)-2-methoxyphenyl]amino}prop-1-yn-1-yl)-1-(2,2,2- |
| | trifluoroethyl)-1H-indol-4-yl]amino}piperidin-1-yl)-3-methoxypropan-2-ol |
| 1800. | 3-(fluoromethoxy)-4-{[3-(4-{[1-(2-hydroxy-3-methoxypropyl)piperidin-4-yl]amino}-1- |
| | (2,2,2-trifluoroethyl)-1H-indol-2-yl)prop-2-yn-1-yl]amino}-N-methylbenzamide |
| 1801. | 1-(4-{[2-(3-{[2-(fluoromethoxy)-4-methanesulfonylphenyl]amino}prop-1-yn-1-yl)-1- |
| | (2,2,2-trifluoroethyl)-1H-indol-4-yl]amino}piperidin-1-yl)-3-methoxypropan-2-ol |
| 1802. | 2-(4-{[3-(4-{[1-(2-hydroxy-3-methoxypropyl)piperidin-4-yl]amino}-1-(2,2,2- |
| | trifluoroethyl)-1H-indol-2-yl)prop-2-yn-1-yl]amino}-3-methoxyphenyl)-2- |
| | methylpropanenitrile |
| 1803. | (2S)-1-(4-{[2-(3-{[2-(fluoromethoxy)-4-methanesulfonylphenyl]amino}prop-1-yn-1-yl)- |
| | 1-(2,2,2-trifluoroethyl)-1H-indol-4-yl]amino}piperidin-1-yl)-3-methoxypropan-2-ol |
| 1804. | (2R)-1-(4-{[2-(3-{[2-(fluoromethoxy)-4-methanesulfonylphenyl]amino}prop-1-yn-1-yl)- |
| | 1-(2,2,2-trifluoroethyl)-1H-indol-4-yl]amino}piperidin-1-yl)-3-methoxypropan-2-ol |
| 1805. | 1-(4-{[2-(3-{[2-(difluoromethoxy)-4-methanesulfonylphenyl]amino}prop-1-yn-1-yl)-1- |
| | (2,2,2-trifluoroethyl)-1H-indol-4-yl]amino}piperidin-1-yl)-3-methoxypropan-2-ol |
| 1806. | 3-(2-hydroxy-3-{4-[(2-{3-[(4-methanesulfonyl-2-methoxyphenyl)amino]prop-1-yn-1-yl}- |
| | 1-(2,2,2-trifluoroethyl)-1H-indol-4-yl)amino]piperidin-1-yl}propoxy)propane-1,2-diol |
| 1807. | 1-{4-[(2-{3-[(5-methanesulfonyl-2-methoxyphenyl)amino]prop-1-yn-1-yl}-1-(2,2,2- |
| | trifluoroethyl)-1H-indol-4-yl)amino]piperidin-1-yl}-3-methoxypropan-2-ol |
| 1808. | 3-(3-(4-((1-(2-hydroxy-3-methoxypropyl)piperidin-4-yl)amino)-1-(2,2,2-trifluoroethyl)- |
| | 1H-indol-2-yl)prop-2-yn-1-yl)-6-(methylsulfonyl)benzo[d]oxazol-2(3H)-one |
| 1809. | 1-{4-[(2-{3-[(4-methanesulfonylphenyl)amino]prop-1-yn-1-yl}-1-(2,2,2-trifluoroethyl)- |
| | 1H-indol-4-yl)amino]piperidin-1-yl}-3-methoxypropan-2-ol |
| 1810. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-3-(2,2,2-trifluoroethoxy)propan-2-ol |
| 1811. | 4-hydroxy-9-[(2-{3-[(4-methanesulfonyl-2-methoxyphenyl)amino]prop-1-yn-1-yl}-1- |
| | (2,2,2-trifluoroethyl)-1H-indol-4-yl)amino]-2-oxa-6λ⁵-azaspiro[5.5]undecan-6-ylium |
| 1812. | 2-{3-[(4-methanesulfonyl-2-methoxyphenyl)amino]prop-1-yn-1-yl}-N-[1-(3- |
| | methoxypropyl)piperidin-4-yl]-1-(2,2,2-trifluoroethyl)-1H-indol-4-amine |
| 1813. | 1-{4-[(2-{3-[(4-methanesulfonyl-2-methoxyphenyl)amino]prop-1-yn-1-yl}-3-methyl-1- |
| | (2,2,2-trifluoroethyl)-1H-indol-4-yl)amino]piperidin-1-yl}-3-methoxypropan-2-ol |
| 1814. | 1-{4-[(2-{3-[(5-fluoro-4-methanesulfonyl-2-methoxyphenyl)amino]prop-1-yn-1-yl}-1- |
| | (2,2,2-trifluoroethyl)-1H-indol-4-yl)amino]piperidin-1-yl}-3-methoxypropan-2-ol |
| 1815. | 2-{3-[(4-methanesulfonyl-2-methoxyphenyl)amino]prop-1-yn-1-yl}-N-[1-(oxetan-3- |
| | yl)piperidin-4-yl]-1-(2,2,2-trifluoroethyl)-1H-indol-4-amine |
| 1816. | 2-(3-((2-methoxy-4-(methylsulfonyl)phenyl)amino)prop-1-yn-1-yl)-N-(1- |
| | (tetrahydrofuran-3-yl)piperidin-4-yl)-1-(2,2,2-trifluoroethyl)-1H-indol-4-amine |
| 1817. | (R)-2-(3-((2-methoxy-4-(methylsulfonyl)phenyl)amino)prop-1-yn-1-yl)-N-(1- |
| | (tetrahydrofuran-3-yl)piperidin-4-yl)-1-(2,2,2-trifluoroethyl)-1H-indol-4-amine |
| 1818. | (S)-2-(3-((2-methoxy-4-(methylsulfonyl)phenyl)amino)prop-1-yn-1-yl)-N-(1- |
| | (tetrahydrofuran-3-yl)piperidin-4-yl)-1-(2,2,2-trifluoroethyl)-1H-indol-4-amine |
| 1819. | 2-(3-((2-methoxy-4-(methylsulfonyl)phenyl)amino)prop-1-yn-1-yl)-N-(1- |
| | ((tetrahydrofuran-2-yl)methyl)piperidin-4-yl)-1-(2,2,2-trifluoroethyl)-1H-indol-4-amine |
| 1820. | 2-fluoro-4-{[3-(4-{[1-(2-hydroxy-3-methoxypropyl)piperidin-4-yl]amino}-1-(2,2,2- |
| | trifluoroethyl)-1H-indol-2-yl)prop-2-yn-1-yl]amino}-5-methoxybenzamide |
| 1821. | 1-methoxy-3-(4-((2-(3-(((3S,4R)-3-methoxytetrahydro-2H-pyran-4-yl)amino)prop-1-yn- |
| | 1-yl)-1-(2,2,2-trifluoroethyl)-1H-indol-4-yl)amino)piperidin-1-yl)propan-2-ol |
| 1822. | 1-(4-{[2-(3-{[4-(cyclopropanesulfonyl)-2-methoxyphenyl]amino}prop-1-yn-1-yl)-1- |
| | (2,2,2-trifluoroethyl)-1H-indol-4-yl]amino}piperidin-1-yl)-3-methoxypropan-2-ol |
| 1823. | 3-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}propanenitrile |
| 1824. | 4-(4-((2-(3-((2-methoxy-4-(methylsulfonyl)phenyl)amino)prop-1-yn-1-yl)-1-(2,2,2- |
| | trifluoroethyl)-1H-indol-4-yl)amino)piperidin-1-yl)butanenitrile |
| 1825. | 2-{3-[(4-methanesulfonyl-2-methoxyphenyl)amino]prop-1-yn-1-yl}-N-{1-[(oxolan-2- |
| | yl)methyl]piperidin-4-yl}-1-(2,2,2-trifluoroethyl)-1H-indol-4-amine |
| 1826. | 1-methoxy-3-(4-{[2-(3-{[2-methoxy-4-(trifluoromethyl)phenyl]amino}prop-1-yn-1-yl)-1- |
| | (2,2,2-trifluoroethyl)-1H-indol-4-yl]amino}piperidin-1-yl)propan-2-ol |
| 1827. | 2-fluoro-4-{[3-(4-{[1-(2-hydroxy-3-methoxypropyl)piperidin-4-yl]amino}-1-(2,2,2- |
| | trifluoroethyl)-1H-indol-2-yl)prop-2-yn-1-yl]amino}-5-methoxy-N-methylbenzamide |
| 1828. | 1-(4-{[2-(3-{[4-(benzenesulfonyl)-2-methoxyphenyl]amino}prop-1-yn-1-yl)-1-(2,2,2- |
| | trifluoroethyl)-1H-indol-4-yl]amino}piperidin-1-yl)-3-methoxypropan-2-ol |
| 1829. | 2-{3-[(4-methanesulfonyl-2-methoxyphenyl)amino]prop-1-yn-1-yl}-N-[1-(2-methoxy-2- |
| | methylpropyl)piperidin-4-yl]-1-(2,2,2-trifluoroethyl)-1H-indol-4-amine |
| 1830. | 2-(4-((2-(3-((2-methoxy-4-(methylsulfonyl)phenyl)amino)prop-1-yn-1-yl)-1-(2,2,2- |
| | trifluoroethyl)-1H-indol-4-yl)amino)piperidin-1-yl)ethan-1-ol |
| 1831. | 1-methoxy-3-(4-{[2-(3-{[2-methoxy-4-(propane-2-sulfonyl)phenyl]amino}prop-1-yn-1- |
| | yl)-1-(2,2,2-trifluoroethyl)-1H-indol-4-yl]amino}piperidin-1-yl)propan-2-ol |
| 1832. | 1-{3-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-3-methoxypropan-2-ol |
| 1833. | 1-methoxy-3-(4-((2-(3-(((3R,4R)-3-methoxytetrahydro-2H-pyran-4-yl)amino)prop-1-yn- |
| | 1-yl)-1-(2,2,2-trifluoroethyl)-1H-indol-4-yl)amino)piperidin-1-yl)propan-2-ol |
| 1834. | 1-{4-[(2-{3-[(4-methanesulfonyl-2-methoxyphenyl)amino]prop-1-yn-1-yl}-1-propyl-1H- |
| | indol-4-yl)amino]piperidin-1-yl}-3-methoxypropan-2-ol |
| 1835. | 1-(4-{[1-(2-fluoroethyl)-2-{3-[(4-methanesulfonyl-2-methoxyphenyl)amino]prop-1-yn-1- |
| | yl}-1H-indol-4-yl]amino}piperidin-1-yl)-3-methoxypropan-2-ol |
| 1836. | 2-(4-{[1-(2-hydroxy-3-methoxypropyl)piperidin-4-yl]amino}-2-{3-[(4-methanesulfonyl- |
| | 2-methoxyphenyl)amino]prop-1-yn-1-yl}-1H-indol-1-yl)acetonitrile |
| 1837. | 1-(4-{[1-(2-chloroethyl)-2-{3-[(4-methanesulfonyl-2-methoxyphenyl)amino]prop-1-yn-1- |
| | yl}-1H-indol-4-yl]amino}piperidin-1-yl)-3-methoxypropan-2-ol |
| 1838. | rac-1-[(3R,4S)-4-[(2-{3-[(4-methanesulfonyl-2-methoxyphenyl)amino]prop-1-yn-1-yl}-1- |
| | (2,2,2-trifluoroethyl)-1H-indol-4-yl)amino]-3-methylpiperidin-1-yl]-3-methoxypropan-2-ol |
| 1839. | rac-1-[(3R,4R)-4-[(2-{3-[(4-methanesulfonyl-2-methoxyphenyl)amino]prop-1-yn-1-yl}- |
| | 1-(2,2,2-trifluoroethyl)-1H-indol-4-yl)amino]-3-methylpiperidin-1-yl]-3-methoxypropan- |
| | 2-ol |
| 1840. | 1-{4-[(2-{3-[(4-methanesulfonyl-2-methylphenyl)amino]prop-1-yn-1-yl}-1-(2,2,2- |
| | trifluoroethyl)-1H-indol-4-yl)amino]piperidin-1-yl}-3-methoxypropan-2-ol |
| 1841. | 1-[4-({1-[(2,2-difluorocyclopropyl)methyl]-2-{3-[(4-methanesulfonyl-2- |
| | methoxyphenyl)amino]prop-1-yn-1-yl}-1H-indol-4-yl}amino)piperidin-1-yl]-3- |
| | methoxypropan-2-ol |
| 1842. | 4-{[3-(4-{[1-(2-hydroxy-3-methoxypropyl)piperidin-4-yl]amino}-1-(2,2,2-trifluoroethyl)- |
| | 1H-indol-2-yl)prop-2-yn-1-yl]amino}-3-methoxybenzoic acid |
| 1843. | 2-{3-[(4-methanesulfonyl-2-methoxyphenyl)amino]prop-1-yn-1-yl}-N-[1-(3- |
| | methanesulfonylpropyl)piperidin-4-yl]-1-(2,2,2-trifluoroethyl)-1H-indol-4-amine |
| 1844. | 4-{[3-(4-{[1-(3-methanesulfonylpropyl)piperidin-4-yl]amino}-1-(2,2,2-trifluoroethyl)- |
| | 1H-indol-2-yl)prop-2-yn-1-yl]amino}-3-methoxybenzoic acid |
| 1845. | 1-(4-{[2-(3-{[4-methanesulfonyl-2-(trifluoromethyl)phenyl]amino}prop-1-yn-1-yl)-1- |
| | (2,2,2-trifluoroethyl)-1H-indol-4-yl]amino}piperidin-1-yl)-3-methoxypropan-2-ol |
| 1846. | 1-(4-{[1-(2,2-difluoroethyl)-2-{3-[(4-methanesulfonyl-2-methoxyphenyl)ino]prop-1-yn- |
| | 1-yl}-1H-indol-4-yl]amino}piperidin-1-yl)-3-methoxypropan-2-ol |
| 1847. | 1-(4-{[2-(3-{[4-methanesulfonyl-2-(trifluoromethoxy)phenyl]amino}prop-1-yn-1-yl)-1- |
| | (2,2,2-trifluoroethyl)-1H-indol-4-yl]amino}piperidin-1-yl)-3-methoxypropan-2-ol |
| 1848. | 1-{4-[(2-{3-[(4-methanesulfonyl-2-methoxyphenyl)amino]prop-1-yn-1-yl}-1-(3,3,3- |
| | trifluoropropyl)-1H-indol-4-yl)amino]piperidin-1-yl}-3-methoxypropan-2-ol |
| 1849. | 1-(4-{[1-(2,2-difluoropropyl)-2-{3-[(4-methanesulfonyl-2-methoxyphenyl)amino]prop-1- |
| | yn-1-yl}-1H-indol-4-yl]amino}piperidin-1-yl)-3-methoxypropan-2-ol |
| 1850. | 1-{4-[(2-{3-[(2-chloro-4-methanesulfonylphenyl)amino]prop-1-yn-1-yl}-1-(2,2,2- |
| | trifluoroethyl)-1H-indol-4-yl)amino]piperidin-1-yl}-3-methoxypropan-2-ol |
| 1851. | 1-{4-[(2-{3-[(2-fluoro-4-methanesulfonyl-6-methoxyphenyl)amino]prop-1-yn-1-yl}-1- |
| | (2,2,2-trifluoroethyl)-1H-indol-4-yl)amino]piperidin-1-yl}-3-methoxypropan-2-ol |
| 1852. | 1-(4-((2-(3-((4-((6-oxa-3-azabicyclo[3.1.1]heptan-3-yl)sulfonyl)-2- |
| | methoxyphenyl)amino)prop-1-yn-1-yl)-1-(2,2,2-trifluoroethyl)-1H-indol-4- |
| | yl)amino)piperidin-1-yl)-3-methoxypropan-2-ol |
| 1853. | (2S)-1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-3-methoxypropan-2-yl propanoate |
| 1854. | 1-{4-[(2-{3-[(4-chloro-2-methoxyphenyl)amino]prop-1-yn-1-yl}-1-(2,2,2-trifluoroethyl)- |
| | 1H-indol-4-yl)amino]piperidin-1-yl}-3-methoxypropan-2-ol |
| 1855. | 2-(2-(4-((2-(3-((2-methoxy-4-(methylsulfonyl)phenyl)amino)prop-1-yn-1-yl)-1-(2,2,2- |
| | trifluoroethyl)-1H-indol-4-yl)amino)piperidin-1-yl)ethoxy)ethan-1-ol |
| 1856. | 4-((3-(4-((1-(2,3-dihydroxypropyl)piperidin-4-yl)amino)-1-(2,2,2-trifluoroethyl)-1H- |
| | indol-2-yl)prop-2-yn-1-yl)amino)-N-(2-hydroxyethyl)-3-methoxybenzenesulfonamide |
| 1857. | 2-{3-[(4-methanesulfonyl-2-methoxyphenyl)amino]prop-1-yn-1-yl}-N-[1-(4-methyl-1,3- |
| | thiazol-2-yl)piperidin-4-yl]-1-(2,2,2-trifluoroethyl)-1H-indol-4-amine |
| 1858. | N-(1-cyclopropylpiperidin-4-yl)-2-{3-[(4-methanesulfonyl-2- |
| | methoxyphenyl)amino]prop-1-yn-1-yl}-1-(2,2,2-trifluoroethyl)-1H-indol-4-amine |
| 1859. | 4-[(3-{4-[(1-cyclopropylpiperidin-4-yl)amino]-1-(2,2,2-trifluoroethyl)-1H-indol-2- |
| | yl}prop-2-yn-1-yl)amino]-3-methoxybenzoic acid |
| 1860. | 2-{3-[(4-methanesulfonyl-2-methoxyphenyl)amino]prop-1-yn-1-yl}-N-{1-[(3R)-oxolan- |
| | 3-yl]piperidin-4-yl}-1-(2,2,2-trifluoroethyl)-1H-indol-4-amine |
| 1861. | 2-{3-[(4-methanesulfonyl-2-methoxyphenyl)amino]prop-1-yn-1-yl}-N-{1-[(3S)-oxolan- |
| | 3-yl]piperidin-4-yl}-1-(2,2,2-trifluoroethyl)-1H-indol-4-amine |
| 1862. | 3-methoxy-4-{[3-(4-{[1-(oxan-4-yl)piperidin-4-yl]amino}-1-(2,2,2-trifluoroethyl)-1H- |
| | indol-2-yl)prop-2-yn-1-yl]amino}benzoic acid |
| 1863. | 3-methoxy-4-((3-(4-((1-(tetrahydro-2H-pyran-4-yl)piperidin-4-yl)amino)-1-(2,2,2- |
| | trifluoroethyl)-1H-indol-2-yl)prop-2-yn-1-yl)amino)benzamide |
| 1864. | N-(2-hydroxyethyl)-3-methoxy-4-((3-(4-((1-(tetrahydro-2H-pyran-4-yl)piperidin-4- |
| | yl)amino)-1-(2,2,2-trifluoroethyl)-1H-indol-2-yl)prop-2-yn-1- |
| | yl)amino)benzenesulfonamide |
| 1865. | 3-methoxy-4-((3-(4-((1′-methyl-[1,4′-bipiperidin]-4-yl)amino)-1-(2,2,2-trifluoroethyl)- |
| | 1H-indol-2-yl)prop-2-yn-1-yl)amino)benzamide |
| 1866. | 3-methoxy-4-((3-(4-((1-(2-(4-methylpiperazin-1-yl)-2-oxoethyl)piperidin-4-yl)amino)-1- |
| | (2,2,2-trifluoroethyl)-1H-indol-2-yl)prop-2-yn-1-yl)amino)benzamide |
| 1867. | 2-{2-[(3-{4-[(1-acetylpiperidin-4-yl)amino]-1-(2,2,2-trifluoroethyl)-1H-indol-2-yl}prop- |
| | 2-yn-1-yl)amino]-5-methanesulfonylphenoxy}acetonitrile |
| 1868. | 4-{[3-(4-{[1-(2-hydroxyacetyl)piperidin-4-yl]amino}-1-(2,2,2-trifluoroethyl)-1H-indol-2- |
| | yl)prop-2-yn-1-yl]amino}-3-methoxy-N,N-dimethylbenzene-1-sulfonamide |
| 1869. | 2-hydroxy-1-{4-[(2-{3-[(2-hydroxy-4-methanesulfonylphenyl)amino]prop-1-yn-1-yl}-1- |
| | (2,2,2-trifluoroethyl)-1H-indol-4-yl)amino]piperidin-1-yl}ethan-1-one |
| 1870. | 2-hydroxy-1-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}ethan-1-one |
| 1871. | N-((3S,4S)-1,3-dimethylpiperidin-4-yl)-2-(3-((2-methoxy-4- |
| | (methylsulfonyl)phenyl)amino)prop-1-yn-1-yl)-1-(2,2,2-trifluoroethyl)-1H-indol-4-amine |
| 1872. | N-((3R,4R)-1,3-dimethylpiperidin-4-yl)-2-(3-((2-methoxy-4- |
| | (methylsulfonyl)phenyl)amino)prop-1-yn-1-yl)-1-(2,2,2-trifluoroethyl)-1H-indol-4-amine |
| 1873. | N-((3R,4R)-1,3-dimethylpiperidin-4-yl)-2-(3-((2-methoxy-4- |
| | (methylsulfonyl)phenyl)amino)prop-1-yn-1-yl)-1-(2,2,2-trifluoroethyl)-1H-indol-4-amine |
| 1874. | N-((3R,4S)-1,3-dimethylpiperidin-4-yl)-2-(3-((2-methoxy-4- |
| | (methylsulfonyl)phenyl)amino)prop-1-yn-1-yl)-1-(2,2,2-trifluoroethyl)-1H-indol-4-amine |
| 1875. | N-((3S,4R)-1,3-dimethylpiperidin-4-yl)-2-(3-((2-methoxy-4- |
| | (methylsulfonyl)phenyl)amino)prop-1-yn-1-yl)-1-(2,2,2-trifluoroethyl)-1H-indol-4-amine |
| 1876. | 1-(4-((2-(3-((2-methoxy-4-(methylsulfonyl)phenyl)amino)prop-1-yn-1-yl)-1-(2,2,2- |
| | trifluoroethyl)-1H-indol-4-yl)amino)-2-rnethylpiperidin-1-yl)ethan-1-one |
| 1877. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]-2-methylpiperidin-1-yl}ethan-1-one |
| 1878. | 4-{[3-(4-{[(2S,4S)-1-acetyl-2-methylpiperidin-4-yl]amino}-1-(2,2,2-trifluoroethyl)-1H- |
| | indol-2-yl)prop-2-yn-1-yl]amino}-3-methoxybenzene-1-sulfonamide |
| 1879. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-2-methoxyethan-1-one |
| 1880. | 2-hydroxy-1-{4-[(2-{3-[(4-methanesulfonyl-2-methoxyphenyl)amino]prop-1-yn-1-yl}-1- |
| | (2,2,2-trifluoroethyl)-1H-indol-4-yl)amino]piperidin-1-yl}propan-1-one |
| 1881. | 2-methoxy-1-(4-((2-(3-((2-methoxy-4-(methylsulfonyl)phenyl)amino)prop-1-yn-1-yl)-1- |
| | (2,2,2-trifluoroethyl)-1H-indol-4-yl)amino)piperidin-1-yl)ethan-1-one |
| 1882. | 3-methoxy-4-((3-(4-((1-(2-methoxyacetyl)piperidin-4-yl)amino)-1-(2,2,2-trifluoroethyl)- |
| | 1H-indol-2-yl)prop-2-yn-1-yl)amino)benzenesulfonamide |
| 1883. | 4-{[3-(4-{[1-(2-hydroxypropanoyl)piperidin-4-yl]amino}-1-(2,2,2-trifluoroethyl)-1H- |
| | indol-2-yl)prop-2-yn-1-yl]amino}-3-methoxybenzene-1-sulfonamide |
| 1884. | 3-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-3-oxopropanenitrile |
| 1885. | 4-{[3-(4-{[1-(2-cyanoacetyl)piperidin-4-yl]amino}-1-(2,2,2-trifluoroethyl)-1H-indol-2- |
| | yl)prop-2-yn-1-yl]amino}-3-methoxybenzene-1-sulfonamide |
| 1886. | 4-{[3-(4-{[1-(2-hydroxyacetyl)piperidin-4-yl]amino}-1-(2,2,2-trifluoroethyl)-1H-indol-2- |
| | yl)prop-2-yn-1-yl]amino}-3-methoxybenzene-1-sulfonamide |
| 1887. | 4-[(3-{4-[(1-acetylpiperidin-4-yl)amino]-1-(2,2,2-trifluoroethyl)-1H-indol-2-yl}prop-2- |
| | yn-1-yl)amino]-3-methoxybenzene-1-sulfonamide |
| 1888. | 2-(dimethylamino)-1-{4-[(2-{3-[(4-methanesulfonyl-2-methoxyphenyl)amino]prop-1-yn- |
| | 1-yl}-1-(2,2,2-trifluoroethyl)-1H-indol-4-yl)amino]piperidin-1-yl}ethan-1-one |
| 1889. | 4-((3-(4-((1-(dimethylglycyl)piperidin-4-yl)amino)-1-(2,2,2-trifluoroethyl)-1H-indol-2- |
| | yl)prop-2-yn-1-yl)amino)-3-methoxybenzenesulfonamide |
| 1890. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}-2-methylpropan-1-one |
| 1891. | 3-methoxy-4-{[3-(4-{[1-(2-methylpropanoyl)piperidin-4-yl]amino}-1-(2,2,2- |
| | trifluoroethyl)-1H-indol-2-yl)prop-2-yn-1-yl]amino}benzene-1-sulfonamide |
| 1892. | 4-((2-(3-((2-methoxy-4-(methylsulfonyl)phenyl)amino)prop-1-yn-1-yl)-1-(2,2,2- |
| | trifluoroethyl)-1H-indol-4-yl)amino)-N,N-dimethylpiperidine-1-carboxamide |
| 1893. | 4-[(3-{4-[(1-acetylpiperidin-4-yl)amino]-1-(2,2,2-trifluoroethyl)-1H-indol-2-yl}prop-2- |
| | yn-1-yl)amino]-3-methoxy-N,N-dimethylbenzene-1-sulfonamide |
| 1894. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]piperidin-1-yl}propan-1-one |
| 1895. | 3-methoxy-4-[(3-{4-[(1-propanoylpiperidin-4-yl)amino]-1-(2,2,2-trifluoroethyl)-1H- |
| | indol-2-yl}prop-2-yn-1-yl)amino]benzene-1-sulfonamide |
| 1896. | 1-(4-{[2-(3-{[2-methoxy-4-(morpholine-4-sulfonyl)phenyl]amino}prop-1-yn-1-yl)-1- |
| | (2,2,2-trifluoroethyl)-1H-indol-4-yl]amino}piperidin-1-yl)ethan-1-one |
| 1897. | 3-methoxy-4-[(3-{4-[(1-{[(4S)-2-oxo-1,3-dioxolan-4-yl]methyl}piperidin-4-yl)amino]-1- |
| | (2,2,2-trifluoroethyl)-1H-indol-2-yl}prop-2-yn-1-yl)amino]benzene-1-sulfonamide |
| 1898. | N-((3-methoxy-4-((3-(4-((1-((2-oxo-1,3-dioxolan-4-yl)methyl)piperidin-4-yl)amino)-1- |
| | (2,2,2-trifluoroethyl)-1H-indol-2-yl)prop-2-yn-1-yl)amino)phenyl)sulfonyl)propionamide |
| 1899. | N-[3-methoxy-4-({3-[4-({1-[(2-oxo-1,3-dioxolan-4-yl)methyl]piperidin-4-yl}amino)-1- |
| | (2,2,2-trifluoroethyl)-1H-indol-2-yl]prop-2-yn-1-yl}amino)benzenesulfonyl]acetamide |
| 1900. | 3-methoxy-N-methyl-4-({3-[4-({1-[(2-oxo-1,3-dioxolan-4-yl)methyl]piperidin-4- |
| | yl}amino)-1-(2,2,2-trifluoroethyl)-1H-indol-2-yl]prop-2-yn-1-yl}amino)benzamide |
| 1901. | 2-(3-((2-methoxy-4-(methylsulfonyl)phenyl)amino)prop-1-yn-1-yl)-N- |
| | (octahydroindolizin-7-yl)-1-(2,2,2-trifluoroethyl)-1H-indol-4-amine |
| 1902. | N-[(7R,8aS)-octahydroindolizin-7-yl]-2-{3-[(4-methanesulfonyl-2- |
| | methoxyphfenyl)amino]prop-1-yn-1-yl}-1-(2,2,2-trifluoroethyl)-1H-indol-4-amine |
| 1903. | N-[(7R,8aR)-octahydroindolizin-7-yl]-2-{3-[(4-methanesulfonyl-2- |
| | methoxyphenyl)amino]prop-1-yn-1-yl}-1-(2,2,2-trifluoroethyl)-1H-indol-4-amine |
| 1904. | rac-(3R,4S)-3-[(2-{3-[(4-methanesulfonyl-2-methoxyphenyl)amino]prop-1-yn-1-yl}-1- |
| | (2,2,2-trifluoroethyl)-1H-indol-4-yl)amino]-1-methylpiperidin-4-ol |
| 1905. | rac-(3R,4R)-4-[(2-{3-[(4-methanesulfonyl-2-methoxyphenyl)amino]prop-1-yn-1-yl}-1- |
| | (2,2,2-trifluoroethyl)-1H-indol-4-yl)amino]-1-methylpiperidin-3-ol |
| 1906. | 3-methoxy-4-((3-(4-((1-(tetrahydro-2H-pyran-4-yl)piperidin-4-yl)amino)-1-(2,2,2- |
| | trifluoroethyl)-1H-indol-2-yl)prop-2-yn-1-yl)amino)benzenesulfonamide |
| 1907. | rac-N-[(3R,4S)-3-fluoro-1-methylpiperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1908. | N-((3S,4S)-3-fluoro-1-methylpiperidin-4-yl)-2-(3-((2-methoxy-4- |
| | (methylsulfonyl)phenyl)amino)prop-1-yn-1-yl)-1-(2,2,2-trifluoroethyl)-1H-indol-4-amine |
| 1909. | N-[(3S,4S)-3-fluoro-1-methylpiperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1910. | N-[(3R,4S)-3-fluoro-1-methylpiperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1911. | N-[(3S,4R)-3-fluoro-1-methylpiperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1912. | N-[(3R,4R)-3-fluoro-1-methylpiperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1913. | rac-4-{[3-(4-{[(3R,4S)-3-fluoro-1-methylpiperidin-4-yl]amino}-1-(2,2,2-trifluoroethyl)- |
| | 1H-indol-2-yl)prop-2-yn-1-yl]amino}-3-methoxybenzene-1-sulfonamide |
| 1914. | rac-4-{[3-(4-{[(3R,4R)-3-fluoro-1-methylpiperidin-4-yl]amino}-1-(2,2,2-trifluoroethyl)- |
| | 1H-indol-2-yl)prop-2-yn-1-yl]amino}-3-methoxybenzene-1-sulfonamide |
| 1915. | rac-methyl 4-{[3-(4-{[(3R,4R)-3-fluoro-1-methylpiperidin-4-yl]amino}-1-(2,2,2- |
| | trifluoroethyl)-1H-indol-2-yl)prop-2-yn-1-yl]amino}-3-methoxybenzoate |
| 1916. | rac-methyl 4-{[3-(4-{[(3R,4S)-3-fluoro-1-methylpiperidin-4-yl]amino}-1-(2,2,2- |
| | trifluoroethyl)-1H-indol-2-yl)prop-2-yn-1-yl]amino}-3-methoxybenzoate |
| 1917. | rac-4-{[3-(4-{[(3R,4S)-3-fluoro-1-methylpiperidin-4-yl]amino}-1-(2,2,2-trifluoroethyl)- |
| | 1H-indol-2-yl)prop-2-yn-1-yl]amino}-3-methoxybenzoic acid |
| 1918. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-3-methoxybenzoic acid |
| 1919. | 4-{[3-(4-{[(3R,4S)-3-fluoro-1-methylpiperidin-4-yl]amino}-1-(2,2,2-trifluoroethyl)-1H- |
| | indol-2-yl)prop-2-yn-1-yl]amino}-3-methoxybenzoic acid |
| 1920. | rac-4-{[3-(4-{[(3R,4S)-3-fluoro-1-methylpiperidin-4-yl]amino}-1-(2,2,2-trifluoroethyl)- |
| | 1H-indol-2-yl)prop-2-yn-1-yl]amino}-3-methoxybenzamide |
| 1921. | rac-4-{[3-(4-{[(3R,4S)-3-fluoro-1-methylpiperidin-4-yl]amino}-1-(2,2,2-trifluoroethyl)- |
| | 1H-indol-2-yl)prop-2-yn-1-yl]amino}-3-methoxy-N-methylbenzamide |
| 1922. | 4-((3-(4-(((3S,4R)-3-fluoro-1-methylpiperidin-4-yl)amino)-1-(2,2,2-trifluoroethyl)-1H- |
| | indol-2-yl)prop-2-yn-1-yl)amino)-3-methoxy-N-methylbenzamide |
| 1923. | rac-4-{[3-(4-{[(3R,4R)-3-fluoro-1-methylpiperidin-4-yl]amino}-1-(2,2,2-trifluoroethyl)- |
| | 1H-indol-2-yl)prop-2-yn-1-yl]amino}-3-methoxy-N-methylbenzamide |
| 1924. | 2-fluoro-4-((3-(4-(((3R,4S)-3-fluoro-1-methylpiperidin-4-yl)amino)-1-(2,2,2- |
| | trifluoroethyl)-1H-indol-2-yl)prop-2-yn-1-yl)amino)-5-methoxy-N-methylbenzamide |
| 1925. | 2-fluoro-4-((3-(4-(((3S,4R)-3-fluoro-1-methylpiperidin-4-yl)amino)-1-(2,2,2- |
| | trifluoroethyl)-1H-indol-2-yl)prop-2-yn-1-yl)amino)-5-methoxy-N-methylbenzamide |
| 1926. | 4-{[3-(4-{[(3R,4R)-3-fluoro-1-methylpiperidin-4-yl]amino}-1-(2,2,2-trifluoroethyl)-1H- |
| | indol-2-yl)prop-2-yn-1-yl]amino}-3-methoxybenzoic acid |
| 1927. | 4-{[3-(4-{[(3S,4S)-3-fluoro-1-methylpiperidin-4-yl]amino}-1-(2,2,2-trifluoroethyl)-1H- |
| | indol-2-yl)prop-2-yn-1-yl]amino}-3-methoxybenzoic acid |
| 1928. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-3-methoxybenzene-1-sulfonamide |
| 1929. | 4-{[3-(4-{[(3R,4S)-3-fluoro-1-methylpiperidin-4-yl]amino}-1-(2,2,2-trifluoroethyl)-1H- |
| | indol-2-yl)prop-2-yn-1-yl]amino}-3-methoxybenzene-1-sulfonamide |
| 1930. | methyl 4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-3-methoxybenzoate |
| 1931. | rac-4-{[3-(4-{[(3R,4R)-3-fluoro-1-methylpiperidin-4-yl]amino}-1-(2,2,2-trifluoroethyl)- |
| | 1H-indol-2-yl)prop-2-yn-1-yl]amino}-3-methoxybenzamide |
| 1932. | methyl 4-{[3-(4-{[(3R,4S)-3-fluoro-1-methylpiperidin-4-yl]amino}-1-(2,2,2- |
| | trifluoroethyl)-1H-indol-2-yl)prop-2-yn-1-yl]amino}-3-methoxybenzoate |
| 1933. | rac-N-[(3R,4S)-3-fluoro-1-(propan-2-yl)piperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1934. | rac-4-{[3-(4-{[(3R,4S)-3-fluoro-1-(propan-2-yl)piperidin-4-yl]amino}-1-(2,2,2- |
| | trifluoroethyl)-1H-indol-2-yl)prop-2-yn-1-yl]amino}-3-methoxybenzoic acid |
| 1935. | rac-ethyl 4-{[3-(4-{[(3R,4S)-3-fluoro-1-(propan-2-yl)piperidin-4-yl]amino}-1-(2,2,2- |
| | trifluoroethyl)-1H-indol-2-yl)prop-2-yn-1-yl]amino}-3-methoxybenzoate |
| 1936. | rac-ethyl 4-{[3-(4-{[(3R,4S)-3-fluoro-1-methylpiperidin-4-yl]amino}-1-(2,2,2- |
| | trifluoroethyl)-1H-indol-2-yl)prop-2-yn-1-yl]amino}-3-methoxybenzoate |
| 1937. | (2R)-1-(acetyloxy)-3-[(3RS,4SR)-3-fluoro-4-[(2-{3-[(2-methoxy-4- |
| | sulfamoylphenyl)amino]prop-1-yn-1-yl}-1-(2,2,2-trifluoroethyl)-1H-indol-4- |
| | yl)amino]piperidin-1-yl]propan-2-yl acetate |
| 1938. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-3-methoxybenzamide |
| 1939. | 4-{[3-(4-{[(3R,4S)-3-fluoro-1-methylpiperidin-4-yl]amino}-1-(2,2,2-trifluoroethyl)-1H- |
| | indol-2-yl)prop-2-yn-1-yl]amino}-3-methoxybenzamide |
| 1940. | N-[(3S,4R)-3-fluoro-1-(propan-2-yl)piperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1941. | N-[(3R,4S)-3-fluoro-1-(propan-2-yl)piperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1942. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-3-methoxy-N-methylbenzamide |
| 1943. | 4-{[3-(4-{[(3R,4S)-3-fluoro-1-methylpiperidin-4-yl]amino}-1-(2,2,2-trifluoroethyl)-1H- |
| | indol-2-yl)prop-2-yn-1-yl]amino}-3-methoxy-N-methylbenzamide |
| 1944. | rac-2-hydroxypropyl 4-{[3-(4-{[(3R,4S)-3-fluoro-1-methylpiperidin-4-yl]amino}-1- |
| | (2,2,2-trifluoroethyl)-1H-indol-2-yl)prop-2-yn-1-yl]amino}-3-methoxybenzoate |
| 1945. | 4-((3-(4-(((3R,4S)-3-fluoro-1-methylpiperidin-4-yl)amino)-1-(2,2,2-trifluoroethyl)-1H- |
| | indol-2-yl)prop-2-yn-1-yl)amino)-N-isopropyl-3-methoxybenzamide |
| 1946. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-3-methoxy-N-(propan-2-yl)benzamide |
| 1947. | 4-{[3-(4-{[(3S,4R)-3-fluoro-1-(propan-2-yl)piperidin-4-yl]amino}-1-(2,2,2- |
| | trifluoroethyl)-1H-indol-2-yl)prop-2-yn-1-yl]amino}-3-methoxybenzoic acid |
| 1948. | 4-{[3-(4-{[(3R,4S)-3-fluoro-1-(propan-2-yl)piperidin-4-yl]amino}-1-(2,2,2- |
| | trifluoroethyl)-1H-indol-2-yl)prop-2-yn-1-yl]amino}-3-methoxybenzoic acid |
| 1949. | rac-2-[(3R,4S)-3-fluoro-4-[(2-{3-[(4-methanesulfonyl-2-methoxyphenyl)amino]prop-1- |
| | yn-1-yl}-1-(2,2,2-trifluoroethyl)-1H-indol-4-yl)amino]piperidin-1-yl]acetamide |
| 1950. | N-[(3S,4R)-1-ethyl-3-fluoropiperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1951. | N-[(3R,4S)-1-ethyl-3-fluoropiperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1952. | rac-4-{[3-(4-{[(3R,4S)-3-fluoro-1-methylpiperidin-4-yl]amino}-1-(2,2,2-trifluoroethyl)- |
| | 1H-indol-2-yl)prop-2-yn-1-yl]amino}-3-methoxy-N-(2-methoxyethyl)benzamide |
| 1953. | rac-4-{[3-(4-{[(3R,4S)-3-fluoro-1-methylpiperidin-4-yl]amino}-1-(2,2,2-trifluoroethyl)- |
| | 1H-indol-2-yl)prop-2-yn-1-yl]amino}-3-methoxy-N,N-dimethylbenzamide |
| 1954. | rac-4-{[3-(4-{[(3R,4S)-3-fluoro-1-methylpiperidin-4-yl]amino}-1-(2,2,2-trifluoroethyl)- |
| | 1H-indol-2-yl)prop-2-yn-1-yl]amino}-N-(2-hydroxy-3-methoxypropyl)-3- |
| | methoxybenzamide |
| 1955. | rac-4-{[3-(4-{[(3R,4S)-3-fluoro-1-methylpiperidin-4-yl]amino}-1-(2,2,2-trifluoroethyl)- |
| | 1H-indol-2-yl)prop-2-yn-1-yl]amino}-3-methoxy-N-(propan-2-yl)benzamide |
| 1956. | rac-4-{[3-(4-{[(3R,4S)-3-fluoro-1-methylpiperidin-4-yl]amino}-1-(2,2,2-trifluoroethyl)- |
| | 1H-indol-2-yl)prop-2-yn-1-yl]amino}-3-methoxy-N-(oxan-4-yl)benzamide |
| 1957. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-3-methoxy-N-(oxan-4-yl)benzamide |
| 1958. | rac-N-[(3R,4S)-1-ethyl-3-fluoropiperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1959. | ethyl 4-{[3-(4-{[(3S,4R)-3-fluoro-1-(propan-2-yl)piperidin-4-yl]amino}-1-(2,2,2- |
| | trifluoroethyl)-1H-indol-2-yl)prop-2-yn-1-yl]amino}-3-methoxybenzoate |
| 1960. | ethyl 4-((3-(4-(((3R,4S)-1-ethyl-3-fluoropiperidin-4-yl)amino)-1-(2,2,2-trifluoroethyl)- |
| | 1H-indol-2-yl)prop-2-yn-1-yl)amino)-3-methoxybenzoate |
| 1961. | ethyl 4-{[3-(4-{[(3S,4R)-1-ethyl-3-fluoropiperidin-4-yl]amino}-1-(2,2,2-trifluoroethyl)- |
| | 1H-indol-2-yl)prop-2-yn-1-yl]amino}-3-methoxybenzoate |
| 1962. | ethyl 4-{[3-(4-{[(3R,4S)-1-ethyl-3-fluoropiperidin-4-yl]amino}-1-(2,2,2-trifluoroethyl)- |
| | 1H-indol-2-yl)prop-2-yn-1-yl]amino}-3-methoxybenzoate |
| 1963. | ethyl 4-{[3-(4-{[(3R,4S)-3-fluoro-1-(propan-2-yl)piperidin-4-yl]amino}-1-(2,2,2- |
| | trifluoroethyl)-1H-indol-2-yl)prop-2-yn-1-yl]amino}-3-methoxybenzoate |
| 1964. | 2-fluoro-4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-5-methoxy-N-methylbenzamide |
| 1965. | 2-fluoro-4-{[3-(4-{[(3R,4S)-3-fluoro-1-methylpiperidin-4-yl]amino}-1-(2,2,2- |
| | trifluoroethyl)-1H-indol-2-yl)prop-2-yn-1-yl]amino}-5-methoxy-N-methylbenzamide |
| 1966. | 4-{[3-(4-{[(3R,4S)-1-ethyl-3-fluoropiperidin-4-yl]amino}-1-(2,2,2-trifluoroethyl)-1H- |
| | indol-2-yl)prop-2-yn-1-yl]amino}-3-methoxybenzoic acid |
| 1967. | 4-{[3-(4-{[(3S,4R)-1-ethyl-3-fluoropiperidin-4-yl]amino}-1-(2,2,2-trifluoroethyl)-1H- |
| | indol-2-yl)prop-2-yn-1-yl]amino}-3-methoxybenzoic acid |
| 1968. | rac-N-(2-{bis[(pyridin-2-yl)methyl]amino}ethyl)-4-{[3-(4-{[(3R,4S)-3-fluoro-1- |
| | methylpiperidin-4-yl]amino}-1-(2,2,2-trifluoroethyl)-1H-indol-2-yl)prop-2-yn-1- |
| | yl]amino}-3-methoxybenzamide |
| 1969. | rac-N-[(3R,4S)-3-fluoro-1-methylpiperidin-4-yl]-2-{3-[(4-methanesulfonyl-2- |
| | methoxyphenyl)amino]prop-1-yn-1-yl}-N-methyl-1-(2,2,2-trifluoroethyl)-1H-indol-4- |
| | amine |
| 1970. | rac-4-((3-(4-(((3R,4S)-3-fluoro-1-methylpiperidin-4-yl)amino)-1-(2,2,2-trifluoroethyl)- |
| | 1H-indol-2-yl)prop-2-yn-1-yl)amino)-3-methoxybenzoic acid |
| 1971. | rac-4-{[3-(4-{[(3R,4R)-3-fluoro-1-methylpiperidin-4-yl]amino}-1-(2,2,2-trifluoroethyl)- |
| | 1H-indol-2-yl)prop-2-yn-1-yl]amino}-3-methoxybenzoic acid |
| 1972. | 2-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-5-methanesulfonylphenol |
| 1973. | 2-{[3-(4-{[(3R,4S)-3-fluoro-1-methylpiperidin-4-yl]amino}-1-(2,2,2-trifluoroethyl)-1H- |
| | indol-2-yl)prop-2-yn-1-yl]amino}-5-methanesulfonylphenol |
| 1974. | rac-6-fluoro-N-[(3R,4S)-3-fluoro-1-methylpiperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1975. | N-(4-{[3-(4-{[(3S,4R)-3-fluoro-1-methylpiperidin-4-yl]amino}-1-(2,2,2-trifluoroethyl)- |
| | 1H-indol-2-yl)prop-2-yn-1-yl]amino}-3-methoxybenzenesulfonyl)acetamide |
| 1976. | N-(4-{[3-(4-{[(3R,4S)-3-fluoro-1-methylpiperidin-4-yl]amino}-1-(2,2,2-trifluoroethyl)- |
| | 1H-indol-2-yl)prop-2-yn-1-yl]amino}-3-methoxybenzenesulfonyl)acetamide |
| 1977. | ethyl 4-{[3-(4-{[(3S,4R)-3-fluoro-1-methylpiperidin-4-yl]amino}-1-(2,2,2-trifluoroethyl)- |
| | 1H-indol-2-yl)prop-2-yn-1-yl]amino}-3-methoxybenzoate |
| 1978. | ethyl 4-{[3-(4-{[(3R,4S)-3-fluoro-1-methylpiperidin-4-yl]amino}-1-(2,2,2-trifluoroethyl)- |
| | 1H-indol-2-yl)prop-2-yn-1-yl]amino}-3-methoxybenzoate |
| 1979. | N-[(3R,4S)-3-fluoro-1-methylpiperidin-4-yl]-2-(3-{[2-(fluoromethoxy)-4- |
| | methanesulfonylphenyl]amino}prop-1-yn-1-yl)-1-(2,2,2-trifluoroethyl)-1H-indol-4-amine |
| 1980. | N-[(3S,4R)-3-fluoro-1-methylpiperidin-4-yl]-2-(3-{[2-(fluoromethoxy)-4- |
| | methanesulfonylphenyl]amino}prop-1-yn-1-yl)-1-(2,2,2-trifluoroethyl)-1H-indol-4-amine |
| 1981. | 2-{3-[(2-ethoxy-4-methanesulfonylphenyl)amino]prop-1-yn-1-yl}-N-[(3S,4R)-3-fluoro-1- |
| | methylpiperidin-4-yl]-1-(2,2,2-trifluoroethyl)-1H-indol-4-amine |
| 1982. | 2-{3-[(2-ethoxy-4-methanesulfonylphenyl)amino]prop-1-yn-1-yl}-N-[(3R,4S)-3-fluoro-1- |
| | methylpiperidin-4-yl]-1-(2,2,2-trifluoroethyl)-1H-indol-4-amine |
| 1983. | 4-{[3-(4-{[(3S,4R)-3-fluoropiperidin-4-yl]amino}-1-(2,2,2-trifluoroethyl)-1H-indol-2- |
| | yl)prop-2-yn-1-yl]amino}-3-methoxybenzene-1-sulfonamide |
| 1984. | 4-{[3-(4-{[(3R,4S)-3-fluoropiperidin-4-yl]amino}-1-(2,2,2-trifluoroethyl)-1H-indol-2- |
| | yl)prop-2-yn-1-yl]amino}-3-methoxybenzene-1-sulfonamide |
| 1985. | N-((3S,4R)-3-fluoropiperidin-4-yl)-2-(3-((2-methoxy-4- |
| | (methylsulfonyl)phenyl)amino)prop-1-yn-1-yl)-1-(2,2,2-trifluoroethyl)-1H-indol-4-amine |
| 1986. | N-((3R,4S)-3-fluoropiperidin-4-yl)-2-(3-((2-methoxy-4- |
| | (methylsulfonyl)phenyl)amino)prop-1-yn-1-yl)-1-(2,2,2-trifluoroethyl)-1H-indol-4-amine |
| 1987. | 4-{[3-(4-{[(3S,4R)-3-fluoropiperidin-4-yl]amino}-1-(2,2,2-trifluoroethyl)-1H-indol-2- |
| | yl)prop-2-yn-1-yl]amino}-3-methoxybenzamide |
| 1988. | 4-((3-(4-(((3R,4S)-3-fluoropiperidin-4-yl)amino)-1-(2,2,2-trifluoroethyl)-1H-indol-2- |
| | yl)prop-2-yn-1-yl)amino)-3-methoxybenzamide |
| 1989. | 4-{[3-(4-{[(3R,4S)-3-fluoropiperidin-4-yl]amino}-1-(2,2,2-trifluoroethyl)-1H-indol-2- |
| | yl)prop-2-yn-1-yl]amino}-3-methoxy-N-methylbenzamide |
| 1990. | 4-{[3-(4-{[(3R,4S)-3-fluoropiperidin-4-yl]amino}-1-(2,2,2-trifluoroethyl)-1H-indol-2- |
| | yl)prop-2-yn-1-yl]amino}-3-methoxybenzoic acid |
| 1991. | 2-[(3S,4R)-3-fluoro-4-[(2-{3-[(4-methanesulfonyl-2-methoxyphenyl)amino]prop-1-yn-1- |
| | yl}-1-(2,2,2-trifluoroethyl)-1H-indol-4-yl)amino]piperidin-1-yl]ethan-1-ol |
| 1992. | 2-[(3R,4S)-3-fluoro-4-[(2-{3-[(4-methanesulfonyl-2-methoxyphenyl)amino]prop-1-yn-1- |
| | yl}-1-(2,2,2-trifluoroethyl)-1H-indol-4-yl)amino]piperidin-1-yl]ethan-1-ol |
| 1993. | 2-(dimethylamino)-1-[(3S,4R)-3-fluoro-4-[(2-{3-[(4-methanesulfonyl-2- |
| | methoxyphenyl)amino]prop-1-yn-1-yl}-1-(2,2,2-trifluoroethyl)-1H-indol-4- |
| | yl)amino]piperidin-1-yl]ethan-1-one |
| 1994. | 2-(dimethylamino)-1-[(3R,4S)-3-fluoro-4-[(2-{3-[(4-methanesulfonyl-2- |
| | methoxyphenyl)amino]prop-1-yn-1-yl}-1-(2,2,2-trifluoroethyl)-1H-indol-4- |
| | yl)amino]piperidin-1-yl]ethan-1-one |
| 1995. | 4-{[3-(4-{[(3S,4R)-3-fluoropiperidin-4-yl]amino}-1-(2,2,2-trifluoroethyl)-1H-indol-2- |
| | yl)prop-2-yn-1-yl]amino}-3-methoxy-N-methylbenzamide |
| 1996. | 4-{[3-(4-{[(3S,4R)-3-fluoropiperidin-4-yl]amino}-1-(2,2,2-trifluoroethyl)-1H-indol-2- |
| | yl)prop-2-yn-1-yl]amino}-3-methoxybenzoic acid |
| 1997. | N-[(3S,4R)-3-fluoro-1-(2-methoxyethyl)piperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1998. | N-[(3R,4S)-3-fluoro-1-(2-methoxyethyl)piperidin-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 1999. | 4-{[3-(4-{[(3S,4R)-3-fluoro-1-[(2R)-2-hydroxy-3-methoxypropyl]piperidin-4-yl]amino}- |
| | 1-(2,2,2-trifluoroethyl)-1H-indol-2-yl)prop-2-yn-1-yl]amino}-3-methoxybenzene-1- |
| | sulfonamide |
| 2000. | 1-[(3S,4R)-3-fluoro-4-[(2-{3-[(4-methanesulfonyl-2-methoxyphenyl)amino]prop-1-yn-1- |
| | yl}-1-(2,2,2-trifluoroethyl)-1H-indol-4-yl)amino]piperidin-1-yl]ethan-1-one |
| 2001. | 1-[(3R,4S)-3-fluoro-4-[(2-{3-[(4-methanesulfonyl-2-methoxyphenyl)amino]prop-1-yn-1- |
| | yl}-1-(2,2,2-trifluoroethyl)-1H-indol-4-yl)amino]piperidin-1-yl]ethan-1-one |
| 2002. | 1-[(3S,4R)-3-fluoro-4-[(2-{3-[(4-methanesulfonyl-2-methoxyphenyl)amino]prop-1-yn-1- |
| | yl}-1-(2,2,2-trifluoroethyl)-1H-indol-4-yl)amino]piperidin-1-yl]propan-1-one |
| 2003. | 1-[(3R,4S)-3-fluoro-4-[(2-{3-[(4-methanesulfonyl-2-methoxyphenyl)amino]prop-1-yn-1- |
| | yl}-1-(2,2,2-trifluoroethyl)-1H-indol-4-yl)amino]piperidin-1-yl]propan-1-one |
| 2004. | 1-[(3S,4R)-3-fluoro-4-[(2-{3-[(4-methanesulfonyl-2-methoxyphenyl)amino]prop-1-yn-1- |
| | yl}-1-(2,2,2-trifluoroethyl)-1H-indol-4-yl)amino]piperidin-1-yl]-2-methoxyethan-1-one |
| 2005. | 1-[(3R,4S)-3-fluoro-4-[(2-{3-[(4-methanesulfonyl-2-methoxyphenyl)amino]prop-1-yn-1- |
| | yl}-1-(2,2,2-trifluoroethyl)-1H-indol-4-yl)amino]piperidin-1-yl]-2-methoxyethan-1-one |
| 2006. | 4-{[3-(4-{[(3R,4S)-3-fluoropiperidin-4-yl]amino}-1-(2,2,2-trifluoroethyl)-1H-indol-2- |
| | yl)prop-2-yn-1-yl]amino}-3-methoxybenzamide |
| 2007. | 4-{[3-(4-{[(3R,4S)-3-fluoro-1-[(2R)-2-hydroxy-3-methoxypropyl]piperidin-4-yl]amino}- |
| | 1-(2,2,2-trifluoroethyl)-1H-indol-2-yl)prop-2-yn-1-yl]amino}-3-methoxybenzene-1- |
| | sulfonamide |
| 2008. | N-[(7S,8R)-7-fluoro-1,4-dioxaspiro[4.5]decan-8-yl]-2-{3-[(4-methanesulfonyl-2- |
| | methoxyphenyl)amino]prop-1-yn-1-yl}-1-(2,2,2-trifluoroethyl)-1H-indol-4-amine |
| 2009. | 1-[(3S,4R)-3-fluoro-4-[(2-{3-[(4-methanesulfonyl-2-methoxyphenyl)amino]prop-1-yn-1- |
| | yl}-1-(2,2,2-trifluoroethyl)-1H-indol-4-yl)amino]piperidin-1-yl]propan-2-ol |
| 2010. | 1-((3S,4S)-3-fluoro-4-((2-(3-((2-methoxy-4-(methylsulfonyl)phenyl)amino)prop-1-yn-1- |
| | yl)-1-(2,2,2-trifluoroethyl)-1H-indol-4-yl)amino)piperidin-1-yl)-3-methoxypropan-2-ol |
| 2011. | 1-((3R,4S)-3-fluoro-4-((2-(3-((2-methoxy-4-(methylsulfonyl)phenyl)amino)prop-1-yn-1- |
| | yl)-1-(2,2,2-trifluoroethyl)-1H-indol-4-yl)amino)piperidin-1-yl)-3-methoxypropan-2-ol |
| 2012. | 4-((3-(4-(((3S,4S)-3-fluoro-1-((R)-2-hydroxy-3-methoxypropyl)piperidin-4-yl)amino)-1- |
| | (2,2,2-trifluoroethyl)-1H-indol-2-yl)prop-2-yn-1-yl)amino)-3-methoxybenzoic acid |
| 2013. | 4-((3-(4-(((3R,4S)-3-fluoro-1-((R)-2-hydroxy-3-methoxypropyl)piperidin-4-yl)amino)-1- |
| | (2,2,2-trifluoroethyl)-1H-indol-2-yl)prop-2-yn-1-yl)amino)-3-methoxybenzoic acid |
| 2014. | 4-((3-(4-(((3R,4S)-3-fluoro-1-((R)-2-hydroxy-3-methoxypropyl)piperidin-4-yl)amino)-1- |
| | (2,2,2-trifluoroethyl)-1H-indol-2-yl)prop-2-yn-1-yl)amino)-3-methoxybenzoic acid |
| 2015. | 4-((3-(4-(((3S,4R)-3-fluoro-1-((R)-2-hydroxy-3-methoxypropyl)piperidin-4-yl)amino)-1- |
| | (2,2,2-trifluoroethyl)-1H-indol-2-yl)prop-2-yn-1-yl)amino)-3-methoxybenzoic acid |
| 2016. | methyl 4-((3-(4-(((3S,4R)-3-fluoro-1-((R)-2-hydroxy-3-methoxypropyl)piperidin-4- |
| | yl)amino)-1-(2,2,2-trifluoroethyl)-1H-indol-2-yl)prop-2-yn-1-yl)amino)-3- |
| | methoxybenzoate |
| 2017. | methyl 4-((3-(4-(((3R,4S)-3-fluoro-1-((R)-2-hydroxy-3-methoxypropyl)piperidin-4- |
| | yl)amino)-1-(2,2,2-trifluoroethyl)-1H-indol-2-yl)prop-2-yn-1-yl)amino)-3- |
| | methoxybenzoate |
| 2018. | (R)-1-((3R,4S)-3-fluoro-4-((2-(3-((2-fluoro-6-methoxy-4- |
| | (methylsulfonyl)phenyl)amino)prop-1-yn-1-yl)-1-propyl-1H-indol-4-yl)amino)piperidin- |
| | 1-yl)-3-methoxypropan-2-ol |
| 2019. | (R)-1-((3R,4S)-4-((1-allyl-2-(3-((2-fluoro-6-methoxy-4- |
| | (methylsulfonyl)phenyl)amino)prop-1-yn-1-yl)-1H-indol-4-yl)amino)-3-fluoropiperidin- |
| | 1-yl)-3-methoxypropan-2-ol |
| 2020. | 4-{[3-(4-{[(3S,4R)-1-[(2R)-2,3-dihydroxypropyl]-3-fluoropiperidin-4-yl]amino}-1-(2,2,2- |
| | trifluoroethyl)-1H-indol-2-yl)prop-2-yn-1-yl]amino}-3-methoxybenzene-1-sulfonamide |
| 2021. | 4-{[3-(4-{[(3R,4S)-1-[(2R)-2,3-dihydroxypropyl]-3-fluoropiperidin-4-yl]amino}-1-(2,2,2- |
| | trifluoroethyl)-1H-indol-2-yl)prop-2-yn-1-yl]amino}-3-methoxybenzene-1-sulfonamide |
| 2022. | (2R)-1-[(3RS,4SR)-3-fluoro-4-[(2-{3-[(4-methanesulfonyl-2-methoxyphenyl)amino]prop- |
| | 1-yn-1-yl}-1-propyl-1H-indol-4-yl)amino]piperidin-1-yl]-3-methoxypropan-2-ol |
| 2023. | 4-((3-(4-(((3S,4S)-3-fluoro-1-((R)-2-hydroxy-3-methoxypropyl)piperidin-4-yl)amino)-1- |
| | (2,2,2-trifluoroethyl)-1H-indol-2-yl)prop-2-yn-1-yl)amino)-3-methoxybenzoic acid |
| 2024. | 4-((3-(4-(((3S,4S)-3-fluoro-1-((R)-2-hydroxy-3-methoxypropyl)piperidin-4-yl)amino)-1- |
| | (2,2,2-trifluoroethyl)-1H-indol-2-yl)prop-2-yn-1-yl)amino)-3-methoxybenzoic acid |
| 2025. | 4-((3-(4-(((3R,4R)-3-fluoro-1-((R)-2-hydroxy-3-methoxypropyl)piperidin-4-yl)amino)-1- |
| | (2,2,2-trifluoroethyl)-1H-indol-2-yl)prop-2-yn-1-yl)amino)-3-methoxybenzoic acid |
| 2026. | (2R)-1-(acetyloxy)-3-[(3R,4S)-3-fluoro-4-[(2-{3-[(2-methoxy-4- |
| | sulfamoylphenyl)amino]prop-1-yn-1-yl}-1-(2,2,2-trifluoroethyl)-1H-indol-4- |
| | yl)amino]piperidin-1-yl]propan-2-yl acetate |
| 2027. | N-[(2R)-2,3-dihydroxypropyl]-4-{[3-(4-{[(3RS,4SR)-3-fluoro-1-[(2R)-2-hydroxy-3- |
| | methoxypropyl]piperidin-4-yl]amino}-1-(2,2,2-trifluoroethyl)-1H-indol-2-yl)prop-2-yn-1- |
| | yl]amino}-3-methoxybenzene-1-sulfonamide |
| 2028. | (2R)-1-(acetyloxy)-3-[(3S,4R)-3-fluoro-4-[(2-{3-[(2-methoxy-4- |
| | sulfamoylphenyl)amino]prop-1-yn-1-yl}-1-(2,2,2-trifluoroethyl)-1H-indol-4- |
| | yl)amino]piperidin-1-yl]propan-2-yl acetate |
| 2029. | rac-N-(2-{bis[(pyridin-2-yl)methyl]amino}ethyl)-2-[(3R,4S)-3-fluoro-4-[(2-{3-[(4- |
| | methanesulfonyl-2-methoxyphenyl)amino]prop-1-yn-1-yl}-1-(2,2,2-trifluoroethyl)-1H- |
| | indol-4-yl)amino]piperidin-1-yl]acetamide |
| 2030. | 2-amino-1-[(3R,4S)-3-fluoro-4-[(2-{3-[(4-methanesulfonyl-2- |
| | methoxyphenyl)amino]prop-1-yn-1-yl}-1-(2,2,2-trifluoroethyl)-1H-indol-4- |
| | yl)amino]piperidin-1-yl]ethan-1-one |
| 2031. | 2-amino-1-[(3S,4R)-3-fluoro-4-[(2-{3-[(4-methanesulfonyl-2- |
| | methoxyphenyl)amino]prop-1-yn-1-yl}-1-(2,2,2-trifluoroethyl)-1H-indol-4- |
| | yl)amino]piperidin-1-yl]ethan-1-one |
| 2032. | 1-[(3R,4S)-3-fluoro-4-[(2-{3-[(4-methanesulfonyl-2-methoxyphenyl)amino]prop-1-yn-1- |
| | yl}-1-(2,2,2-trifluoroethyl)-1H-indol-4-yl)amino]piperidin-1-yl]propan-2-ol |
| 2033. | 3-(4-(((3S,4R)-3-fluoro-1-methylpiperidin-4-yl)amino)-1-(2,2,2-trifluoroethyl)-1H-indol- |
| | 2-yl)prop-2-yn-1-ol |
| 2034. | (4-((3-(4-(((3S,4R)-3-fluoro-1-methylpiperidin-4-yl)amino)-1-(2,2,2-trifluoroethyl)-1H- |
| | indol-2-yl)prop-2-yn-1-yl)amino)-3-methoxybenzoyl)glycine |
| 2035. | methyl 4-{[3-(4-{[(3S,4R)-3-fluoro-1-(2-methoxyethyl)piperidin-4-yl]amino}-1-(2,2,2- |
| | trifluoroethyl)-1H-indol-2-yl)prop-2-yn-1-yl]amino}-3-methoxybenzoate |
| 2036. | 4-{[3-(4-{[(3S,4R)-3-fluoro-1-(2-methoxyethyl)piperidin-4-yl]amino}-1-(2,2,2- |
| | trifluoroethyl)-1H-indol-2-yl)prop-2-yn-1-yl]amino}-3-methoxybenzoic acid |
| 2037. | methyl 4-{[3-(4-{[(3S,4R)-3-fluoro-1-(2-hydroxyethyl)piperidin-4-yl]amino}-1-(2,2,2- |
| | trifluoroethyl)-1H-indol-2-yl)prop-2-yn-1-yl]amino}-3-methoxybenzoate |
| 2038. | methyl 4-{[3-(4-{[(3S,4R)-3-fluoro-1-(2-hydroxypropyl)piperidin-4-yl]amino}-1-(2,2,2- |
| | trifluoroethyl)-1H-indol-2-yl)prop-2-yn-1-yl]amino}-3-methoxybenzoate |
| 2039. | methyl 4-{[3-(4-{[(3S,4R)-1-(carbamoylmethyl)-3-fluoropiperidin-4-yl]amino}-1-(2,2,2- |
| | trifluoroethyl)-1H-indol-2-yl)prop-2-yn-1-yl]amino}-3-methoxybenzoate |
| 2040. | methyl 4-{[3-(4-{[(3R,4S)-3-fluoro-1-(2-methoxyethyl)piperidin-4-yl]amino}-1-(2,2,2- |
| | trifluoroethyl)-1H-indol-2-yl)prop-2-yn-1-yl]amino}-3-methoxybenzoate |
| 2041. | methyl 4-{[3-(4-{[(3R,4S)-3-fluoro-1-(2-hydroxyethyl)piperidin-4-yl]amino}-1-(2,2,2- |
| | trifluoroethyl)-1H-indol-2-yl)prop-2-yn-1-yl]amino}-3-methoxybenzoate |
| 2042. | 4-{[3-(4-{[(3R,4S)-3-fluoro-1-(2-methoxyethyl)piperidin-4-yl]amino}-1-(2,2,2- |
| | trifluoroethyl)-1H-indol-2-yl)prop-2-yn-1-yl]amino}-3-methoxybenzoic acid |
| 2043. | methyl 4-{[3-(4-{[(3R,4S)-3-fluoro-1-(2-hydroxypropyl)piperidin-4-yl]amino}-1-(2,2,2- |
| | trifluoroethyl)-1H-indol-2-yl)prop-2-yn-1-yl]amino}-3-methoxybenzoate |
| 2044. | N-[(3S,4R)-3-fluoro-1-methylpiperidin-4-yl]-2-{3-[(2-methoxy-4-{2-oxa-6- |
| | azaspiro[3.3]heptane-6-carbonyl}phenyl)amino]prop-1-yn-1-yl}-1-(2,2,2-trifluoroethyl)- |
| | 1H-indol-4-amine |
| 2045. | N-[(3S,4R)-3-fluoro-1-methylpiperidin-4-yl]-2-{3-[(2-methoxy-4-{7-oxa-2- |
| | azaspiro[3.5]nonane-2-carbonyl}phenyl)amino]prop-1-yn-1-yl}-1-(2,2,2-trifluoroethyl)- |
| | 1H-indol-4-amine |
| 2046. | 4-{[3-(4-{[(3S,4R)-3-fluoro-1-(2-hydroxyethyl)piperidin-4-yl]amino}-1-(2,2,2- |
| | trifluoroethyl)-1H-indol-2-yl)prop-2-yn-1-yl]amino}-3-methoxybenzoic acid |
| 2047. | N-[(3S,4R)-3-fluoro-1-methylpiperidin-4-yl]-2-(3-{[2-methoxy-4-(morpholine-4- |
| | carbonyl)phenyl]amino}prop-1-yn-1-yl)-1-(2,2,2-trifluoroethyl)-1H-indol-4-amine |
| 2048. | N-[(3S,4R)-3-fluoro-1-methylpiperidin-4-yl]-2-(3-{[2-methoxy-4-(4-methylpiperazine-1- |
| | carbonyl)phenyl]amino}prop-1-yn-1-yl)-1-(2,2,2-trifluoroethyl)-1H-indol-4-amine |
| 2049. | 4-{[3-(4-{[(3R,4S)-3-fluoro-1-(2-hydroxypropyl)piperidin-4-yl]amino}-1-(2,2,2- |
| | trifluoroethyl)-1H-indol-2-yl)prop-2-yn-1-yl]amino}-3-methoxybenzoic acid |
| 2050. | 4-{[3-(4-{[(3R,4S)-3-fluoro-1-(2-hydroxyethyl)piperidin-4-yl]amino}-1-(2,2,2- |
| | trifluoroethyl)-1H-indol-2-yl)prop-2-yn-1-yl]amino}-3-methoxybenzoic acid |
| 2051. | methyl 4-{[3-(4-{[(3S,4R)-1-[(dimethylcarbamoyl)methyl]-3-fluoropiperidin-4- |
| | yl]amino}-1-(2,2,2-trifluoroethyl)-1H-indol-2-yl)prop-2-yn-1-yl]amino}-3- |
| | methoxybenzoate |
| 2052. | methyl 4-{[3-(4-{[(3R,4S)-1-[(dimethylcarbamoyl)methyl]-3-fluoropiperidin-4- |
| | yl]amino}-1-(2,2,2-trifluoroethyl)-1H-indol-2-yl)prop-2-yn-1-yl]amino}-3- |
| | methoxybenzoate |
| 2053. | methyl 4-{[3-(4-{[(3R,4S)-1-(carbamoylmethyl)-3-fluoropiperidin-4-yl]amino}-1-(2,2,2- |
| | trifluoroethyl)-1H-indol-2-yl)prop-2-yn-1-yl]amino}-3-methoxybenzoate |
| 2054. | 2-((3R,4S)-3-fluoro-4-((2-(3-((2-methoxy-4-(methylsulfonyl)phenyl)amino)prop-1-yn-1- |
| | yl)-1-(2,2,2-trifluoroethyl)-1H-indol-4-yl)amino)piperidin-1-yl)acetamide |
| 2055. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-3-methoxy-N-(1,3-thiazol-2-yl)benzamide |
| 2056. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-3-methoxy-N-(1-methylpiperidin-4-yl)benzamide |
| 2057. | 1-(4-{[3-(4-{[(3S,4R)-3-fluoro-1-methylpiperidin-4-yl]amino}-1-(2,2,2-trifluoroethyl)- |
| | 1H-indol-2-yl)prop-2-yn-1-yl]amino}-3-methoxybenzoyl)piperidin-4-ol |
| 2058. | 4-{[3-(4-{[(3S,4R)-1-[(dimethylcarbamoyl)methyl]-3-fluoropiperidin-4-yl]amino}-1- |
| | (2,2,2-trifluoroethyl)-1H-indol-2-yl)prop-2-yn-1-yl]amino}-3-methoxybenzoic acid |
| 2059. | 4-{[3-(4-{[(3S,4R)-3-fluoro-1-(2-hydroxypropyl)piperidin-4-yl]amino}-1-(2,2,2- |
| | trifluoroethyl)-1H-indol-2-yl)prop-2-yn-1-yl]amino}-3-methoxybenzoic acid |
| 2060. | tert-butyl (3S,4R)-4-{[2-(3-{[4-(ethanesulfonyl)-2-methoxyphenyl]amino}prop-1-yn-1- |
| | yl)-1-(2,2,2-trifluoroethyl)-1H-indol-4-yl]amino}-3-fluoropiperidine-1-carboxylate |
| 2061. | 2-(3-((4-(ethylsulfonyl)-2-methoxyphenyl)amino)prop-1-yn-1-yl)-N-((3S,4R)-3- |
| | fluoropiperidin-4-yl)-1-(2,2,2-trifluoroethyl)-1H-indol-4-amine |
| 2062. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-3-methoxy-N-[2-(morpholin-4-yl)ethyl]benzamide |
| 2063. | 4-{[3-(4-{[(3R,4S)-1-(carbamoylmethyl)-3-fluoropiperidin-4-yl]amino}-1-(2,2,2- |
| | trifluoroethyl)-1H-indol-2-yl)prop-2-yn-1-yl]amino}-3-methoxybenzoic acid |
| 2064. | 4-{[3-(4-{[(3S,4R)-1-(carbamoylmethyl)-3-fluoropiperidin-4-yl]amino]-1-(2,2,2- |
| | trifluoroethyl)-1H-indol-2-yl)prop-2-yn-1-yl]amino}-3-methoxybenzoic acid |
| 2065. | 4-{[3-(4-{[(3R,4S)-1-[(dimethylcarbamoyl)methyl]-3-fluoropiperidin-4-yl]amino}-1- |
| | (2,2,2-trifluoroethyl)-1H-indol-2-yl)prop-2-yn-1-yl]amino}-3-methoxybenzoic acid |
| 2066. | tert-butyl (3S,4R)-4-[(2-{3-[(2-ethoxy-4-methanesulfonylphenyl)amino]prop-1-yn-1-yl}- |
| | 1-(2,2,2-trifluoroethyl)-1H-indol-4-yl)amino]-3-fluoropiperidine-1-carboxylate |
| 2067. | (2S)-2-[(4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-3- |
| | methoxyphenyl)formamido]pentanedioic acid |
| 2068. | (2S)-4-carbamoyl-2-[(4-{[3-(4-{[(3S,4R)-3-fluoro-1-methylpiperidin-4-yl]amino}-1- |
| | (2,2,2-trifluoroethyl)-1H-indol-2-yl)prop-2-yn-1-yl]amino}-3- |
| | methoxyphenyl)formamido]butanoic acid |
| 2069. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-3-methoxybenzene-1-sulfonic acid |
| 2070. | 1,5-dimethyl (2S)-2-[(4-{[3-(4-{[(3S,4R)-3-fluoro-1-methylpiperidin-4-yl]amino}-1- |
| | (2,2,2-trifluoroethyl)-1H-indol-2-yl)prop-2-yn-1-yl]amino}-3- |
| | methoxyphenyl)formamido]pentanedioate |
| 2071. | 2-[3-({4-[4-(dimethylamino)piperidine-1-carbonyl]-2-methoxyphenyl}amino)prop-1-yn- |
| | 1-yl]-N-[(3S,4R)-3-fluoro-1-methylpiperidin-4-yl]-1-(2,2,2-trifluoroethyl)-1H-indol-4- |
| | amine |
| 2072. | 4-{[3-(4-{[(3S,4R)-1-(carboxymethyl)-3-fluoropiperidin-4-yl]amino}-1-(2,2,2- |
| | trifluoroethyl)-1H-indol-2-yl)prop-2-yn-1-yl]amino}-3-methoxybenzoic acid |
| 2073. | 2-(3-{[4-(ethanesulfonyl)-2-methoxyphenyl]amino}prop-1-yn-1-yl)-N-[(3S,4R)-3- |
| | fluoropiperidin-4-yl]-1-(2,2,2-trifluoroethyl)-1H-indol-4-amine |
| 2074. | (1R,2R,4S)-2-fluoro-N¹-(2-{3-[(4-methanesulfonyl-2-methoxyphenyl)amino]prop-1-yn- |
| | 1-yl}-1-(2,2,2-trifluoroethyl)-1H-indol-4-yl)cyclohexane-1,4-diamine |
| 2075. | (1R,2R,4S)-2-fluoro-N¹-(2-{3-[(4-methanesulfonyl-2-methoxyphenyl)amino]prop-1-yn- |
| | 1-yl}-1-(2,2,2-trifluoroethyl)-1H-indol-4-yl)-N⁴-methylcyclohexane-1,4-diamine |
| 2076. | (1S,3R,4R)-3-fluoro-N⁴-(2-{3-[(4-methanesulfonyl-2-methoxyphenyl)amino]prop-1-yn- |
| | 1-yl}-1-(2,2,2-trifluoroethyl)-1H-indol-4-yl)-N¹,N¹-dimethylcyclohexane-1,4-diamine |
| 2077. | 2-(4-{[3-(4-{[(3S,4R)-3-fluoro-1-methylpiperidin-4-yl]amino}-1-(2,2,2-trifluoroethyl)- |
| | 1H-indol-2-yl)prop-2-yn-1-yl]amino}-3-methoxybenzenesulfonyl)ethan-1-ol |
| 2078. | N-ethyl-4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-3-methoxybenzamide |
| 2079. | 2-{3-[(2-ethoxy-4-methanesulfonylphenyl)amino]prop-1-yn-1-yl}-N-[(3S,4R)-3- |
| | fluoropiperidin-4-yl]-1-(2,2,2-trifluoroethyl)-1H-indol-4-amine |
| 2080. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-3-methoxy-N,N-dimethylbenzamide |
| 2081. | N-ethyl-4-((3-(4-(((3S,4R)-3-fluoro-1-methylpiperidin-4-yl)amino)-1-(2,2,2- |
| | trifluoroethyl)-1H-indol-2-yl)prop-2-yn-1-yl)amino)-3-methoxy-N-methylbenzamide |
| 2082. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-3-methoxy-N-methyl-N-(propan-2-yl)benzamide |
| 2083. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-3-methoxy-N-(2-methoxyethyl)benzamide |
| 2084. | N-[2-(diethylamino)ethyl]-4-{[3-(4-{[(3S,4R)-3-fluoro-1-methylpiperidin-4-yl]amino}-1- |
| | (2,2,2-trifluoroethyl)-1H-indol-2-yl)prop-2-yn-1-yl]amino}-3-methoxybenzamide |
| 2085. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-N-(2-hydroxyethyl)-3-methoxybenzamide |
| 2086. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-3-methoxy-N-[(2R,3R,4R,5S,6R)-2,4,5-trihydroxy-6- |
| | (hydroxymethyl)oxan-3-yl]benzamide |
| 2087. | 4-((3-(4-(((3S,4R)-3-fluoro-1-methylpiperidin-4-yl)amino)-1-(2,2,2-trifluoroethyl)-1H- |
| | indol-2-yl)prop-2-yn-1-yl)amino)-N-(1-hydroxypropan-2-yl)-3-methoxybenzamide |
| 2088. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-N-[(2R)-1-hydroxypropan-2-yl]-3-methoxybenzamide |
| 2089. | N-[3-(4-{[(3S,4R)-3-fluoro-1-methylpiperidin-4-yl]amino}-1-(2,2,2-trifluoroethyl)-1H- |
| | indol-2-yl)prop-2-yn-1-yl]cyclopropanecarboxamide |
| 2090. | (1R,2R)-N-[3-(4-{[(3S,4R)-3-fluoro-1-methylpiperidin-4-yl]amino}-1-(2,2,2- |
| | trifluoroethyl)-1H-indol-2-yl)prop-2-yn-1-yl]-2-phenylcyclopropane-1-carboxamide |
| 2091. | N-[3-(4-{[(3S,4R)-3-fluoro-1-methylpiperidin-4-yl]amino}-1-(2,2,2-trifluoroethyl)-1H- |
| | indol-2-yl)prop-2-yn-1-yl]-1-methyl-1H-pyrrole-3-carboxamide |
| 2092. | 1-ethyl-N-[3-(4-{[(3S,4R)-3-fluoro-1-methylpiperidin-4-yl]amino}-1-(2,2,2- |
| | trifluoroethyl)-1H-indol-2-yl)prop-2-yn-1-yl]-1H-pyrrole-3-carboxamide |
| 2093. | 1-tert-butyl-N-[3-(4-{[(3S,4R)-3-fluoro-1-methylpiperidin-4-yl]amino}-1-(2,2,2- |
| | trifluoroethyl)-1H-indol-2-yl)prop-2-yn-1-yl]-1H-pyrrole-3-carboxamide |
| 2094. | methyl (2S)-4-carbamoyl-2-[(4-{[3-(4-{[(3S,4R)-3-fluoro-1-methylpiperidin-4- |
| | yl]amino}-1-(2,2,2-trifluoroethyl)-1H-indol-2-yl)prop-2-yn-1-yl]amino}-3- |
| | methoxyphenyl)formamido]butanoate |
| 2095. | 4-((3-(4-(((3S,4R)-3-fluoro-1-methylpiperidin-4-yl)amino)-1-(2,2,2-trifluoroethyl)-1H- |
| | indol-2-yl)prop-2-yn-1-yl)amino)-N-((R)-2-hydroxypropyl)-3-methoxybenzamide |
| 2096. | rac-4-{[3-(4-{[(3R,4S)-1-ethyl-3-fluoropiperidin-4-yl]amino}-1-(2,2,2-trifluoroethyl)-1H- |
| | indol-2-yl)prop-2-yn-1-yl]amino}-3-methoxybenzoic acid |
| 2097. | 4-((3-(4-(((3S,4R)-3-fluoro-1-methylpiperidin-4-yl)amino)-1-(2,2,2-trifluoroethyl)-1H- |
| | indol-2-yl)prop-2-yn-1-yl)amino)-N-((S)-2-hydroxypropyl)-3-methoxybenzamide |
| 2098. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-N-(2-hydroxy-3-methoxypropyl)-3-methoxybenzamide |
| 2099. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-N-[(2S)-2-hydroxypropyl]-3-methoxybenzamide |
| 2100. | N-(2,3-dihydroxypropyl)-4-{[3-(4-{[(3S,4R)-3-fluoro-1-methylpiperidin-4-yl]amino}-1- |
| | (2,2,2-trifluoroethyl)-1H-indol-2-yl)prop-2-yn-1-yl]amino}-3-methoxybenzamide |
| 2101. | N-[(2R)-2,3-dihydroxypropyl]-4-{[3-(4-{[(3S,4R)-3-fluoro-1-methylpiperidin-4- |
| | yl]amino}-1-(2,2,2-trifluoroethyl)-1H-indol-2-yl)prop-2-yn-1-yl]amino}-3- |
| | methoxybenzamide |
| 2102. | N-[(2S)-2,3-dihydroxypropyl]-4-{[3-(4-{[(3S,4R)-3-fluoro-1-methylpiperidin-4- |
| | yl]amino}-1-(2,2,2-trifluoroethyl)-1H-indol-2-yl)prop-2-yn-1-yl]amino}-3- |
| | methoxybenzamide |
| 2103. | N-(1,5-dihydroxypentan-3-yl)-4-{[3-(4-{[(3S,4R)-3-fluoro-1-methylpiperidin-4- |
| | yl]amino}-1-(2,2,2-trifluoroethyl)-1H-indol-2-yl)prop-2-yn-1-yl]amino}-3- |
| | methoxybenzamide |
| 2104. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-N-(3-hydroxy-2-methoxypropyl)-3-methoxybenzamide |
| 2105. | 1-[(4-{[3-(4-{[(3S,4R)-3-fluoro-1-methylpiperidin-4-yl]amino}-1-(2,2,2-trifluoroethyl)- |
| | 1H-indol-2-yl)prop-2-yn-1-yl]amino}-3-methoxyphenyl)formamido]-3-[(2- |
| | methylpropanoyl)oxy]propan-2-yl 2-methylpropanoate |
| 2106. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-3-methoxy-N-[(2-oxo-1,3-dioxolan-4- |
| | yl)methyl]benzamide |
| 2107. | 4-{[3-(4-{[(3S,4R)-3-fluoro-1-methylpiperidin-4-yl]amino}-1-(2,2,2-trifluoroethyl)-1H- |
| | indol-2-yl)prop-2-yn-1-yl]amino}-N-(2-methanesulfonylethyl)-3-methoxybenzamide |
| 2108. | 1-(acetyloxy)-3-[(4-{[3-(4-{[(3S,4R)-3-fluoro-1-methylpiperidin-4-yl]amino}-1-(2,2,2- |
| | trifluoroethyl)-1H-indol-2-yl)prop-2-yn-1-yl]amino}-3- |
| | methoxyphenyl)formamido]propan-2-yl acetate |
| 2109. | 1-[(4-{[3-(4-{[(3S,4R)-3-fluoro-1-methylpiperidin-4-yl]amino}-1-(2,2,2-trifluoroethyl)- |
| | 1H-indol-2-yl)prop-2-yn-1-yl]amino}-3-methoxyphenyl)formamido]-3- |
| | (propanoyloxy)propan-2-yl propanoate |
| 2110. | 2-[(4-{[3-(4-{[(3S,4R)-3-fluoro-1-methylpiperidin-4-yl]amino}-1-(2,2,2-trifluoroethyl)- |
| | 1H-indol-2-yl)prop-2-yn-1-yl]amino}-3-methoxyphenyl)formamido]propyl 2- |
| | methylpropanoate |
| 2111. | (S)-5-ethoxy-2-(4-((3-(4-(((3S,4R)-3-fluoro-1-methylpiperidin-4-yl)amino)-1-(2,2,2- |
| | trifluoroethyl)-1H-indol-2-yl)prop-2-yn-1-yl)amino)-3-methoxybenzamido)-5- |
| | oxopentanoic acid |
| 2112. | (4-((3-(4-(((3S,4R)-3-fluoro-1-methylpiperidin-4-yl)amino)-1-(2,2,2-trifluoroethyl)-1H- |
| | indol-2-yl)prop-2-yn-1-yl)amino)-3-methoxybenzoyl)-L-glutamine |
| 2113. | (S)-2-(4-((3-(4-(((3S,4R)-3-fluoro-1-methylpiperidin-4-yl)amino)-1-(2,2,2-trifluoroethyl)- |
| | 1H-indol-2-yl)prop-2-yn-1-yl)amino)-3-methoxybenzamido)-5-methoxy-5-oxopentanoic |
| | acid |
| 2114. | (S)-1-((3S,4S)-3-fluoro-4-((2-(3-((2-methoxy-4-(methylsulfonyl)phenyl)amino)prop-1- |
| | yn-1-yl)-1-(2,2,2-trifluoroethyl)-1H-indol-4-yl)amino)piperidin-1-yl)-3-methoxypropan- |
| | 2-ol |
| 2115. | (S)-1-((3R,4R)-3-fluoro-4-((2-(3-((2-methoxy-4-(methylsulfonyl)phenyl)amino)prop-1- |
| | yn-1-yl)-1-(2,2,2-trifluoroethyl)-1H-indol-4-yl)amino)piperidin-1-yl)-3-methoxypropan- |
| | 2-ol |
| 2116. | (S)-1-((3R,4S)-3-fluoro-4-((2-(3-((2-methoxy-4-(methylsulfonyl)phenyl)amino)prop-1- |
| | yn-1-yl)-1-(2,2,2-trifluoroethyl)-1H-indol-4-yl)amino)piperidin-1-yl)-3-methoxypropan- |
| | 2-ol |
| 2117. | (S)-1-((3S,4R)-3-fluoro-4-((2-(3-((2-methoxy-4-(methylsulfonyl)phenyl)amino)prop-1- |
| | yn-1-yl)-1-(2,2,2-trifluoroethyl)-1H-indol-4-yl)amino)piperidin-1-yl)-3-methoxypropan- |
| | 2-ol |
| 2118. | 1-((3S,4S)-3-fluoro-4-((2-(3-((2-methoxy-4-(methylsulfonyl)phenyl)amino)prop-1-yn-1- |
| | yl)-1-(2,2,2-trifluoroethyl)-1H-indol-4-yl)amino)piperidin-1-yl)-3-methoxypropan-2-ol |
| 2119. | 1-((3R,4S)-3-fluoro-4-((2-(3-((2-methoxy-4-(methylsulfonyl)phenyl)amino)prop-1-yn-1- |
| | yl)-1-(2,2,2-trifluoroethyl)-1H-indol-4-yl)amino)piperidin-1-yl)-3-methoxypropan-2-ol |
| 2120. | (R)-1-((3S,4S)-3-fluoro-4-((2-(3-((2-methoxy-4-(methylsulfonyl)phenyl)amino)prop-1- |
| | yn-1-yl)-1-(2,2,2-trifluoroethyl)-1H-indol-4-yl)amino)piperidin-1-yl)-3-methoxypropan- |
| | 2-ol |
| 2121. | (R)-1-((3R,4R)-3-fluoro-4-((2-(3-((2-methoxy-4-(methylsulfonyl)phenyl)amino)prop-1- |
| | yn-1-yl)-1-(2,2,2-trifluoroethyl)-1H-indol-4-yl)amino)piperidin-1-yl)-3-methoxypropan- |
| | 2-ol |
| 2122. | (R)-1-((3R,4S)-3-fluoro-4-((2-(3-((2-methoxy-4-(methylsulfonyl)phenyl)amino)prop-1- |
| | yn-1-yl)-1-(2,2,2-trifluoroethyl)-1H-indol-4-yl)amino)piperidin-1-yl)-3-methoxypropan- |
| | 2-ol |
| 2123. | (R)-1-((3S,4R)-3-fluoro-4-((2-(3-((2-methoxy-4-(methylsulfonyl)phenyl)amino)prop-1- |
| | yn-1-yl)-1-(2,2,2-trifluoroethyl)-1H-indol-4-yl)amino)piperidin-1-yl)-3-methoxypropan- |
| | 2-ol |
| 2124. | 4-((3-(4-(((3R,4S)-3-fluoro-1-((R)-2-hydroxy-3-methoxypropyl)piperidin-4-yl)amino)-1- |
| | (2,2,2-trifluoroethyl)-1H-indol-2-yl)prop-2-yn-1-yl)amino)-3- |
| | methoxybenzenesulfonamide |
| 2125. | 1-(3,3-difluoro-4-((2-(3-((2-methoxy-4-(methylsulfonyl)phenyl)amino)prop-1-yn-1-yl)-1- |
| | (2,2,2-trifluoroethyl)-1H-indol-4-yl)amino)piperidin-1-yl)-3-methoxypropan-2-ol |
| 2126. | 2-{3-[(4-methanesulfonyl-2-methoxyphenyl)amino]prop-1-yn-1-yl}-N-(1-methylazepan- |
| | 4-yl)-1-(2,2,2-trifluoroethyl)-1H-indol-4-amine |
| 2127. | 1-{4-[(2-{3-[(4-methanesulfonyl-2-methoxyphenyl)amino]prop-1-yn-1-yl}-1-(2,2,2- |
| | trifluoroethyl)-1H-indol-4-yl)amino]azepan-1-yl}-3-methoxypropan-2-ol |
| 2128. | 2-{3-[(4-methanesulfonyl-2-methoxyphenyl)amino]prop-1-yn-1-yl}-N-{8-methyl-8- |
| | azabicyclo[3.2.1]octan-3-yl}-1-(2,2,2-trifluoroethyl)-1H-indol-4-amine |
| 2129. | 2-(3-{[4-methanesulfonyl-2-(trifluoromethoxy)phenyl]amino}prop-1-yn-1-yl)-N-(oxan-4- |
| | yl)-1-(2,2,2-trifluoroethyl)-1H-indol-4-amine |
| 2130. | 3-methoxy-4-[(3-{4-[(oxan-4-yl)amino]-1-(2,2,2-trifluoroethyl)-1H-indol-2-yl}prop-2- |
| | yn-1-yl)amino]-N-(1,2-oxazol-3-yl)benzene-1-sulfonamide |
| 2131. | 3-methoxy-N-(oxan-4-yl)-4-[(3-{4-[(oxan-4-yl)amino]-1-(2,2,2-trifluoroethyl)-1H-indol- |
| | 2-yl}prop-2-yn-1-yl)amino]benzene-1-sulfonamide |
| 2132. | 3-methoxy-N-(5-methyl-1,2-oxazol-3-yl)-4-[(3-{4-[(oxan-4-yl)amino]-1-(2,2,2- |
| | trifluoroethyl)-1H-indol-2-yl}prop-2-yn-1-yl)amino]benzene-1-sulfonamide |
| 2133. | 2-{4-methoxy-5-[(3-{4-[(oxan-4-yl)amino]-1-(2,2,2-trifluoroethyl)-1H-indol-2-yl}prop-2- |
| | yn-1-yl)amino]pyridin-2-yl}-2-methylpropanenitrile |
| 2134. | N-(2-hydroxyethyl)-3-methoxy-N-methyl-4-[(3-{4-[(oxan-4-yl)amino]-1-(2,2,2- |
| | trifluoroethyl)-1H-indol-2-yl}prop-2-yn-1-yl)amino]benzene-1-sulfonamide |
| 2135. | 6-fluoro-2-{3-[(4-methanesulfonyl-2-methoxyphenyl)amino]prop-1-yn-1-yl}-N-(oxan-4- |
| | yl)-1-(2,2,2-trifluoroethyl)-1H-indol-4-amine |
| 2136. | 4-[(3-{6-fluoro-4-[(oxan-4-yl)amino]-1-(2,2,2-trifluoroethyl)-1H-indol-2-yl}prop-2-yn-1- |
| | yl)amino]-3-methoxybenzene-1-sulfonamide |
| 2137. | 3-methoxy-N,N-dimethyl-4-[(3-{4-[(oxan-4-yl)amino]-1-(2,2,2-trifluoroethyl)-1H-indol- |
| | 2-yl}prop-2-yn-1-yl)amino]benzene-1-sulfonamide |
| 2138. | 3-methoxy-N-(2-methoxyethyl)-N-methyl-4-[(3-{4-[(oxan-4-yl)amino]-1-(2,2,2- |
| | trifluoroethyl)-1H-indol-2-yl}prop-2-yn-1-yl)amino]benzene-1-sulfonamide |
| 2139. | 1-(4-{3-methoxy-4-[(3-{4-[(oxan-4-yl)amino]-1-(2,2,2-trifluoroethyl)-1H-indol-2- |
| | yl}prop-2-yn-1-yl)amino]benzenesulfonyl}piperazin-1-yl)ethan-1-one |
| 2140. | 2-(3-{[2-methoxy-4-(morpholine-4-sulfonyl)phenyl]amino}prop-1-yn-1-yl)-N-(oxan-4- |
| | yl)-1-(2,2,2-trifluoroethyl)-1H-indol-4-amine |
| 2141. | 3-methoxy-N-methyl-4-[(3-{4-[(oxan-4-yl)amino]-1-(2,2,2-trifluoroethyl)-1H-indol-2- |
| | yl}prop-2-yn-1-yl)amino]benzamide |
| 2142. | 3-methoxy-4-((3-(4-((tetrahydro-2H-pyran-4-yl)amino)-1-(2,2,2-trifluoroethyl)-1H-indol- |
| | 2-yl)prop-2-yn-1-yl)amino)benzoic acid |
| 2143. | methyl 3-methoxy-4-((3-(4-((tetrahydro-2H-pyran-4-yl)amino)-1-(2,2,2-trifluoroethyl)- |
| | 1H-indol-2-yl)prop-2-yn-1-yl)amino)benzoate |
| 2144. | 3-methoxy-4-((3-(4-((tetrahydro-2H-pyran-4-yl)amino)-1-(2,2,2-trifluoroethyl)-1H-indol- |
| | 2-yl)prop-2-yn-1-yl)amino)benzamide |
| 2145. | 3-methoxy-N-(2-methoxyethyl)-N-methyl-4-((3-(4-((tetrahydro-2H-pyran-4-yl)amino)-1- |
| | (2,2,2-trifluoroethyl)-1H-indol-2-yl)prop-2-yn-1-yl)amino)benzenesulfonamide |
| 2146. | N,N-bis(2-hydroxyethyl)-3-methoxy-4-[(3-{4-[(oxan-4-yl)amino]-1-(2,2,2- |
| | trifluoroethyl)-1H-indol-2-yl}prop-2-yn-1-yl)amino]benzene-1-sulfonamide |
| 2147. | 3-methoxy-N-methyl-4-[(3-{4-[(oxan-4-yl)amino]-1-(2,2,2-trifluoroethyl)-1H-indol-2- |
| | yl}prop-2-yn-1-yl)amino]benzene-1-sulfonamide |
| 2148. | 5-methanesulfonyl-2-[(3-{4-[(oxan-4-yl)amino]-1-(2,2,2-trifluoroethyl)-1H-indol-2- |
| | yl}prop-2-yn-1-yl)amino]phenol |
| 2149. | 3-methoxy-4-[(3-{5-methoxy-4-[(oxan-4-yl)amino]-1-(2,2,2-trifluoroethyl)-1H-indol-2- |
| | yl}prop-2-yn-1-yl)amino]benzene-1-sulfonamide |
| 2150. | 2-[3-({2-methoxy-4-[(4-methylpiperazin-1-yl)sulfonyl]phenyl}amino)prop-1-yn-1-yl]-N- |
| | (oxan-4-yl)-1-(2,2,2-trifluoroethyl)-1H-indol-4-amine |
| 2151. | 2-{3-[(4-methanesulfonyl-2-methoxyphenyl)amino]prop-1-yn-1-yl}-5-methoxy-N-(oxan- |
| | 4-yl)-1-(2,2,2-trifluoroethyl)-1H-indol-4-amine |
| 2152. | 3-methoxy-4-[(3-{6-methoxy-4-[(oxan-4-yl)amino]-1-(2,2,2-trifluoroethyl)-1H-indol-2- |
| | yl}prop-2-yn-1-yl)amino]benzene-1-sulfonamide |
| 2153. | 5-methanesulfonyl-2-[(3-{4-[(oxan-4-yl)amino]-1-(2,2,2-trifluoroethyl)-1H-indol-2- |
| | yl}prop-2-yn-1-yl)amino]phenyl propanoate |
| 2154. | N-(2-hydroxyethyl)-3-methoxy-4-((3-(4-((tetrahydro-2H-pyran-4-yl)amino)-1-(2,2,2- |
| | trifluoroethyl)-1H-indol-2-yl)prop-2-yn-1-yl)amino)benzenesulfonamide |
| 2155. | 2-{3-[(4-methanesulfonyl-2-methoxyphenyl)amino]prop-1-yn-1-yl}-N-[(2S,4R)-2- |
| | methyloxan-4-yl]-1-(2,2,2-trifluoroethyl)-1H-indol-4-amine |
| 2156. | 2-{3-[(4-methanesulfonyl-2-methoxyphenyl)amino]prop-1-yn-1-yl}-N-[(2S,4S)-2- |
| | methyloxan-4-yl]-1-(2,2,2-trifluoroethyl)-1H-indol-4-amine |
| 2157. | 3-methoxy-4-{[3-(4-{[(2S,4R)-2-methyloxan-4-yl]amino}-1-(2,2,2-trifluoroethyl)-1H- |
| | indol-2-yl)prop-2-yn-1-yl]amino}benzene-1-sulfonamide |
| 2158. | 3-methoxy-4-{[3-(4-{[(2S,4S)-2-methyloxan-4-yl]amino}-1-(2,2,2-trifluoroethyl)-1H- |
| | indol-2-yl)prop-2-yn-1-yl]amino}benzene-1-sulfonamide |
| 2159. | 3-methoxy-4-{[3-(4-{[(2S,4R)-2-methyloxan-4-yl]amino}-1-(2,2,2-trifluoroethyl)-1H- |
| | indol-2-yl)prop-2-yn-1-yl]amino}benzamide |
| 2160. | 3-methoxy-4-{[3-(4-{[(2S,4S)-2-methyloxan-4-yl]amino}-1-(2,2,2-trifluoroethyl)-1H- |
| | indol-2-yl)prop-2-yn-1-yl]amino}benzamide |
| 2161. | rac-N-[(3R,4R)-3-fluorooxan-4-yl]-2-{3-[(4-methanesulfonyl-2- |
| | methoxyphenyl)amino]prop-1-yn-1-yl}-1-(2,2,2-trifluoroethyl)-1H-indol-4-amine |
| 2162. | 2-(3-((2-methoxy-4-(methylsulfonyl)phenyl)amino)prop-1-yn-1-yl)-N-((3R,4S)-3- |
| | methoxytetrahydro-2H-pyran-4-yl)-1-(2,2,2-trifluoroethyl)-1H-indol-4-amine |
| 2163. | 2-(3-((2-methoxy-4-(methylsulfonyl)phenyl)amino)prop-1-yn-1-yl)-N-((3S,4S)-3- |
| | methoxytetrahydro-2H-pyran-4-yl)-1-(2,2,2-trifluoroethyl)-1H-indol-4-amine |
| 2164. | N-[(3S,4S)-3-fluorooxan-4-yl]-2-{3-[(4-methanesulfonyl-2-methoxyphenyl)amino]prop- |
| | 1-yn-1-yl}-1-(2,2,2-trifluoroethyl)-1H-indol-4-amine |
| 2165. | N-[(3R,4R)-3-fluorooxan-4-yl]-2-{3-[(4-methanesulfonyl-2-methoxyphenyl)amino]prop- |
| | 1-yn-1-yl}-1-(2,2,2-trifluoroethyl)-1H-indol-4-amine |
| 2166. | 2-{3-[(4-methanesulfonyl-2-methoxyphenyl)amino]prop-1-yn-1-yl}-N-[(2R,4R,6S)-2,6- |
| | dimethyloxan-4-yl]-1-(2,2,2-trifluoroethyl)-1H-indol-4-amine |
| 2167. | 2-{3-[(4-methanesulfonyl-2-methoxyphenyl)amino]prop-1-yn-1-yl}-N-[(2R,4S,6S)-2,6- |
| | dimethyloxan-4-yl]-1-(2,2,2-trifluoroethyl)-1H-indol-4-amine |
| 2168. | 3-methoxy-4-{[3-(4-{[(2R,4R,6S)-2,6-dimethyloxan-4-yl]amino}-1-(2,2,2-trifluoroethyl)- |
| | 1H-indol-2-yl)prop-2-yn-1-yl]amino}benzamide |
| 2169. | 3-methoxy-4-{[3-(4-{[(2R,4S,6S)-2,6-dimethyloxan-4-yl]amino}-1-(2,2,2-trifluoroethyl)- |
| | 1H-indol-2-yl)prop-2-yn-1-yl]amino}benzamide |
| 2170. | 3-methoxy-4-{[3-(4-{[(2R,4S,6S)-2,6-dimethyloxan-4-yl]amino}-1-(2,2,2-trifluoroethyl)- |
| | 1H-indol-2-yl)prop-2-yn-1-yl]amino}benzene-1-sulfonamide |
| 2171. | 3-methoxy-4-{[3-(4-{[(2R,4R,6S)-2,6-dimethyloxan-4-yl]amino}-1-(2,2,2-trifluoroethyl)- |
| | 1H-indol-2-yl)prop-2-yn-1-yl]amino}benzene-1-sulfonamide |
| 2172. | 4-((2-(3-((4-methoxypyridin-3-yl)amino)prop-1-yn-1-yl)-1-(2,2,2-trifluoroethyl)-1H- |
| | indol-4-yl)amino)tetrahydro-2H-thiopyran 1,1-dioxide |
| 2173. | 2-{5-[(3-{4-[(1,1-dioxo-1λ⁶-thian-4-yl)amino]-1-(2,2,2-trifluoroethyl)-1H-indol-2- |
| | yl}prop-2-yn-1-yl)amino]-4-methoxypyridin-2-yl}-2-methylpropanenitrile |
| 2174. | 4-{[2-(3-{[4-methanesulfonyl-2-(trifluoromethoxy)phenyl]amino}prop-1-yn-1-yl)-1- |
| | (2,2,2-trifluoroethyl)-1H-indol-4-yl]amino}-1λ⁶-thiane-1,1-dione |
| 2175. | 4-{[2-(3-{[2-methoxy-4-(morpholine-4-sulfonyl)phenyl]amino}prop-1-yn-1-yl)-1-(2,2,2- |
| | trifluoroethyl)-1H-indol-4-yl]amino}-1λ⁶-thiane-1,1-dione |
| 2176. | 4-({2-[3-({2-[2-(dimethylamino)ethoxy]-4-methanesulfonylphenyl}amino)prop-1-yn-1- |
| | yl]-1-(2,2,2-trifluoroethyl)-1H-indol-4-yl}amino)-1λ⁶-thiane-1,1-dione |
| 2177. | 4-[(3-{4-[(1,1-dioxo-1λ⁶-thian-4-yl)amino]-1-(2,2,2-trifluoroethyl)-1H-indol-2-yl}prop-2- |
| | yn-1-yl)amino]-N-(2-hydroxyethyl)-3-methoxy-N-methylbenzene-1-sulfonamide |
| 2178. | 4-[(3-{4-[(1,1-dioxo-1λ⁶-thian-4-yl)amino]-1-(2,2,2-trifluoroethyl)-1H-indol-2-yl}prop-2- |
| | yn-1-yl)amino]-3-methoxy-N-(1,2-oxazol-3-yl)benzene-1-sulfonamide |
| 2179. | 4-[(3-{4-[(1,1-dioxo-1λ⁶-thian-4-yl)amino]-1-(2,2,2-trifluoroethyl)-1H-indol-2-yl}prop-2- |
| | yn-1-yl)amino]-3-methoxy-N-(5-methyl-1,2-oxazol-3-yl)benzene-1-sulfonamide |
| 2180. | 4-[(3-{4-[(1,1-dioxo-1λ⁶-thian-4-yl)amino]-1-(2,2,2-trifluoroethyl)-1H-indol-2-yl}prop-2- |
| | yn-1-yl)amino]-3-methoxy-N-(oxan-4-yl)benzene-1-sulfonamide |
| 2181. | N-(2,3-dihydroxypropyl)-4-[(3-{4-[(1,1-dioxo-1λ⁶-thian-4-yl)amino]-1-(2,2,2- |
| | trifluoroethyl)-1H-indol-2-yl}prop-2-yn-1-yl)amino]-3-methoxybenzene-1-sulfonamide |
| 2182. | N-[2-(dimethylamino)ethyl]-4-[(3-{4-[(1,1-dioxo-1λ⁶-thian-4-yl)amino]-1-(2,2,2- |
| | trifluoroethyl)-1H-indol-2-yl}prop-2-yn-1-yl)amino]-3-methoxy-N-methylbenzene-1- |
| | sulfonamide |
| 2183. | 4-[(3-{4-[(1,1-dioxo-1λ⁶-thian-4-yl)amino]-1-(2,2,2-trifluoroethyl)-1H-indol-2-yl}prop-2- |
| | yn-1-yl)amino]-3-methoxy-N,N-dimethylbenzene-1-sulfonamide |
| 2184. | 4-({2-[3-({4-[(4-acetylpiperazin-1-yl)sulfonyl]-2-methoxyphenyl}amino)prop-1-yn-1-yl]- |
| | 1-(2,2,2-trifluoroethyl)-1H-indol-4-yl}amino)-1λ⁶-thiane-1,1-dione |
| 2185. | 4-[(3-{4-[(1,1-dioxo-1λ⁶-thian-4-yl)amino]-1-(2,2,2-trifluoroethyl)-1H-indol-2-yl}prop-2- |
| | yn-1-yl)amino]-3-methoxy-N-(2-methoxyethyl)-N-methylbenzene-1-sulfonamide |
| 2186. | 4-[(2-{3-[(2-methoxy-4-{2-oxa-6-azaspiro[3.3]heptane-6-sulfonyl}phenyl)amino]prop-1- |
| | yn-1-yl}-1-(2,2,2-trifluoroethyl)-1H-indol-4-yl)amino]-1λ⁶-thiane-1,1-dione |
| 2187. | 4-({2-[3-({2-methoxy-4-[(4-methylpiperazin-1-yl)sulfonyl]phenyl}amino)prop-1-yn-1- |
| | yl]-1-(2,2,2-trifluoroethyl)-1H-indol-4-yl}amino)-1λ⁶-thiane-1,1-dione |
| 2188. | 4-[(3-{4-[(1,1-dioxo-1λ⁶-thian-4-yl)amino]-1-(2,2,2-trifluoroethyl)-1H-indol-2-yl}prop-2- |
| | yn-1-yl)amino]-3-methoxy-N-methylbenzamide |
| 2189. | 4-((2-(3-((4-methoxy-6-(methylsulfonyl)pyridin-3-yl)amino)prop-1-yn-1-yl)-1-(2,2,2- |
| | trifluoroethyl)-1H-indol-4-yl)amino)tetrahydro-2H-thiopyran 1,1-dioxide |
| 2190. | 4-((2-(3-((4-(methylsulfonyl)-2-(2,2,2-trifluoroethoxy)phenyl)amino)prop-1-yn-1-yl)-1- |
| | (2,2,2-trifluoroethyl)-1H-indol-4-yl)amino)tetrahydro-2H-thiopyran 1,1-dioxide |
| 2191. | 2-(2-((3-(4-((1,1-dioxidotetrahydro-2H-thiopyran-4-yl)amino)-1-(2,2,2-trifluoroethyl)- |
| | 1H-indol-2-yl)prop-2-yn-1-yl)amino)-5-(methylsulfonyl)phenoxy)acetamide |
| 2192. | 2-(2-((3-(4-((1,1-dioxidotetrahydro-2H-thiopyran-4-yl)amino)-1-(2,2,2-trifluoroethyl)- |
| | 1H-indol-2-yl)prop-2-yn-1-yl)amino)-5-(methylsulfonyl)phenoxy)acetamide |

TABLE 2
Aryl-linked indole compounds of the disclosure.

| Mol # | IUPAC name |
|---|---|
| 2193. | 4-{4-[(1,1-dioxo-1λ⁶-thian-4-yl)amino]-1-(2,2,2-trifluoroethyl)-1H-indol-2- |
| | yl}benzamide |
| 2194. | 4-({2-[4-(aminomethyl)phenyl]-1-(2,2,2-trifluoroethyl)-1H-indol-4-yl}amino)- |
| | 1λ⁶-thiane-1,1-dione |
| 2195. | 4-[(2-{4-[(methylamino)methyl]phenyl}-1-(2,2,2-trifluoroethyl)-1H-indol-4- |
| | yl)amino]-1λ⁶-thiane-1,1-dione |
| 2196. | tert-butyl N-[(4-{4-[(1,1-dioxo-1λ⁶-thian-4-yl)amino]-1-(2,2,2-trifluoroethyl)-1H- |
| | indol-2-yl}phenyl)methyl]-N-methylcarbamate |
| 2197. | 4-{4-[(1,1-dioxo-1λ⁶-thian-4-yl)amino]-1-(2,2,2-trifluoroethyl)-1H-indol-2-yl}-N- |
| | methylbenzamide |
| 2198. | tert-butyl N-[(4-{4-[(1,1-dioxo-1λ⁶-thian-4-yl)amino]-1-(2,2,2-trifluoroethyl)-1H- |
| | indol-2-yl}phenyl)methyl]carbamate |
| 2199. | 2-(5-{[(4-{4-[(1,1-dioxo-1λ⁶-thian-4-yl)amino]-1-(2,2,2-trifluoroethyl)-1H-indol- |
| | 2-yl}phenyl)methyl]amino}pyridin-2-yl)-2-methylpropanenitrile |
| 2200. | 4-{[2-(4-{[(4-methanesulfonylphenyl)amino]methyl}phenyl)-1-(2,2,2- |
| | trifluoroethyl)-1H-indol-4-yl]amino}-1λ⁶-thiane-1,1-dione |
| 2201. | 4-[(2-{4-[(phenylamino)methyl]phenyl}-1-(2,2,2-trifluoroethyl)-1H-indol-4- |
| | yl)amino]-1λ⁶-thiane-1,1-dione |
| 2202. | 3-{4-[(1,1-dioxo-1λ⁶-thian-4-yl)amino]-1-(2,2,2-trifluoroethyl)-1H-indol-2- |
| | yl}benzonitrile |
| 2203. | 4-{[2-(2-fluoro-4-methylphenyl)-1-(2,2,2-trifluoroethyl)-1H-indol-4-yl]amino}- |
| | 1λ⁶-thiane-1,1-dione |
| 2204. | 4-{[2-(3-chlorophenyl)-1-(2,2,2-trifluoroethyl)-1H-indol-4-yl]amino}-1λ⁶-thiane- |
| | 1,1-dione |
| 2205. | -{[2-(3-methoxyphenyl)-1-(2,2,2-trifluoroethyl)-1H-indol-4-yl]amino}-1λ⁶-thiane- |
| | 1,1-dione |
| 2206. | 4-{[2-(4-chlorophenyl)-1-(2,2,2-trifluoroethyl)-1H-indol-4-yl]amino}-1λ⁶-thiane- |
| | 1,1-dione |
| 2207. | 4-tert-butyl-N-[(4-{4-[(1,1-dioxo-1λ⁶-thian-4-yl)amino]-1-(2,2,2-trifluoroethyl)- |
| | 1H-indol-2-yl}phenyl)methyl]benzamide |
| 2208. | 4-cyano-N-[(4-{4-[(1,1-dioxo-1λ⁶-thian-4-yl)amino]-1-(2,2,2-trifluoroethyl)-1H- |
| | indol-2-yl}phenyl)methyl]benzamide |
| 2209. | 4-chloro-N-[(4-{4-[(1,1-dioxo-1λ⁶-thian-4-yl)amino]-1-(2,2,2-trifluoroethyl)-1H- |
| | indol-2-yl}phenyl)methyl]benzamide |
| 2210. | 3-[6-(1-cyano-1-methylethyl)pyridin-3-yl]-1-[(4-{4-[(1,1-dioxo-1λ⁶-thian-4- |
| | yl)amino]-1-(2,2,2-trifluoroethyl)-1H-indol-2-yl}phenyl)methyl]urea |
| 2211. | 3-[(4-{4-[(1,1-dioxo-1λ⁶-thian-4-yl)amino]-1-(2,2,2-trifluoroethyl)-1H-indol-2- |
| | yl}phenyl)methyl]-1-phenylurea |
| 2212. | 3-{4-[(1,1-dioxo-1λ⁶-thian-4-yl)amino]-1-(2,2,2-trifluoroethyl)-1H-indol-2- |
| | yl}benzoic acid |
| 2213. | 4-({2-[3-(dimethylamino)phenyl]-1-(2,2,2-trifluoroethyl)-1H-indol-4-yl}amino)- |
| | 1λ⁶-thiane-1,1-dione |
| 2214. | 3-{4-[(1,1-dioxo-1λ⁶-thian-4-yl)amino]-1-(2,2,2-trifluoroethyl)-1H-indol-2-yl}-N- |
| | methylbenzamide |
| 2215. | 4-{4-[(11-dioxo-1λ⁶-thian-4-yl)amino]-1-(2,2,2-trifluoroethyl)-1H-indol-2- |
| | yl}benzoic acid |
| 2216. | 4-[(2-{4-[(morpholin-4-yl)methyl]phenyl}-1-(2,2,2-trifluoroethyl)-1H-indol-4- |
| | yl)amino]-1λ⁶-thiane-1,1-dione |
| 2217. | methyl N-(3-{4-[(1,1-dioxo-1λ⁶-thian-4-yl)amino]-1-(2,2,2-trifluoroethyl)-1H- |
| | indol-2-yl}phenyl)carbamate |
| 2218. | 1-(4-{4-[(1,1-dioxo-1λ⁶-thian-4-yl)amino]-1-(2,2,2-trifluoroethyl)-1H-indol-2- |
| | yl}phenyl)cyclopropane-1-carbonitrile |
| 2219. | 4-({2-[4-(hydroxymethyl)phenyl]-1-(2,2,2-trifluoroethyl)-1H-indol-4-yl}amino)- |
| | 1λ⁶-thiane-1,1-dione |
| 2220. | 1-[(4-{4-[(1,1-dioxo-1λ⁶-thian-4-yl)amino]-1-(2,2,2-trifluoroethyl)-1H-indol-2- |
| | yl}phenyl)methyl]-3-(4-methanesulfonylphenyl)urea |
| 2221. | 4-{[2-(4-{[(6-methanesulfonylpyridin-3-yl)amino]methyl}phenyl)-1-(2,2,2- |
| | trifluoroethyl)-1H-indol-4-yl]amino}-1λ⁶-thiane-1,1-dione |
| 2222. | 2-(4-{[(4-methanesulfonylphenyl)amino]methyl}phenyl)-N-[1-(oxan-4- |
| | yl)piperidin-4-yl]-1-(2,2,2-trifluoroethyl)-1H-indol-4-amine |
| 2223. | 2-(4-{[(6-methylpyridin-3-yl)amino]methyl}phenyl)-N-[1-(oxan-4-yl)piperidin-4- |
| | yl]-1-(2,2,2-trifluoroethyl)-1H-indol-4-amine |
| 2224. | 2-(4-{[(4-chlorophenyl)amino]methyl}phenyl)-N-[1-(oxan-4-yl)piperidin-4-yl]-1- |
| | (2,2,2-trifluoroethyl)-1H-indol-4-amine |
| 2225. | 2-(4-{[(4-methoxyphenyl)amino]methyl}phenyl)-N-[1-(oxan-4-yl)piperidin-4-yl]- |
| | 1-(2,2,2-trifluoroethyl)-1H-indol-4-amine |
| 2226. | 2-(4-{[(3-chlorophenyl)amino]methyl}phenyl)-N-[1-(oxan-4-yl)piperidin-4-yl]-1- |
| | (2,2,2-trifluoroethyl)-1H-indol-4-amine |
| 2227. | 6-methyl-N-{[4-(5-{[(1-methylpiperidin-4-yl)amino]methyl}-1-(2,2,2- |
| | trifluoroethyl)-1H-indol-2-yl)phenyl]methyl}pyridin-3-amine |
| 2228. | N-{[2-(4-{[(4-methanesulfonylphenyl)amino]methyl}phenyl)-1-(2,2,2- |
| | trifluoroethyl)-1H-indol-5-yl]methyl}-1-methylpiperidin-4-amine |
| 2229. | 2-(5-amino-[1,1′-biphenyl]-3-yl)-N-(1-methylpiperidin-4-yl)-1-(2,2,2- |
| | trifluoroethyl)-1H-indol-4-amine |
| 2230. | 2-{4-[amino(phenyl)methyl]phenyl}-N-(1-methylpiperidin-4-yl)-1-(2,2,2- |
| | trifluoroethyl)-1H-indol-4-amine |
| 2231. | 2-(4-(amino(cyclohexyl)methyl)phenyl)-N-(1-methylpiperidin-4-yl)-1-(2,2,2- |
| | trifluoroethyl)-1H-indol-4-amine |
| 2232. | 2-{4-[(cyclopentylamino)methyl]phenyl}-N-(1-methylpiperidin-4-yl)-1-(2,2,2- |
| | trifluoroethyl)-1H-indol-4-amine |
| 2233. | 2-(4-{1-[(4-methanesulfonylphenyl)amino]ethyl}phenyl)-N-(1-methylpiperidin-4- |
| | yl)-1-(2,2,2-trifluoroethyl)-1H-indol-4-amine |
| 2234. | (+/−)-2-{4-[(cyclopropylamino)methyl]phenyl}-N-[(3R,4S)-3-fluoro-1- |
| | methylpiperidin-4-yl]-1-(2,2,2-trifluoroethyl)-1H-indol-4-amine |
| 2235. | (+/−)-N-[(3R,4S)-3-fluoro-1-methylpiperidin-4-yl]-2-(4-{[(4-methanesulfonyl-2- |
| | methoxyphenyl)amino]methyl}phenyl)-1-(2,2,2-trifluoroethyl)-1H-indol-4-amine |
| 2236. | N-[(3R,4S)-3-fluoro-1-methylpiperidin-4-yl]-2-(4-{[(4- |
| | methanesulfonylphenyl)amino]methyl}phenyl)-1-(2,2,2-trifluoroethyl)-1H-indol- |
| | 4-amine |
| 2237. | (+/−)-N-{[4-(4-{[(3R,4S)-3-fluoro-1-methylpiperidin-4-yl]amino}-1-(2,2,2- |
| | trifluoroethyl)-1H-indol-2-yl)phenyl]methyl}benzamide |
| 2238. | (+/−)-N-{[4-(4-{[(3R,4S)-3-fluoro-1-methylpiperidin-4-yl]amino}-1-(2,2,2- |
| | trifluoroethyl)-1H-indol-2-yl)phenyl]methyl}cyclopropanecarboxamide |
| 2239. | 1-methoxy-3-(4-{[2-(3-methyl-2H-indazol-6-yl)-1-(2,2,2-trifluoroethyl)-1H-indol- |
| | 4-yl]amino}piperidin-1-yl)propan-2-ol |
| 2240. | 1-(4-{[2-(2H-indazol-6-yl)-1-(2,2,2-trifluoroethyl)-1H-indol-4- |
| | yl]amino}piperidin-1-yl)-3-methoxypropan-2-ol |
| 2241. | 4-{[1-(2-hydroxy-3-methoxypropyl)piperidin-4-yl]amino}-1-(2,2,2-trifluoroethyl)- |
| | 2′,3′-dihydro-1H,1′H-[2,6′-biindol]-2′-one |
| 2242. | 4-(4-((1-(2-hydroxy-3-methoxypropyl)piperidin-4-yl)amino)-1-(2,2,2- |
| | trifluoroethyl)-1H-indol-2-yl)isoindolin-1-one |
| 2243. | N-[3-(4-{[1-(2-hydroxy-3-methoxypropyl)piperidin-4-yl]amino}-1-(2,2,2- |
| | trifluoroethyl)-1H-indol-2-yl)phenyl]acetamide |

TABLE 3
Heteroaryl-linked indole compounds of the disclosure

| Mol # | IUPAC name |
|---|---|
| 2244. | 4-((2-(6-methoxypyridin-3-yl)-1-(2,2,2-trifluoroethyl)-1H-indol-4- |
| | yl)amino)tetrahydro-2H-thiopyran 1,1-dioxide-indol-4-amine |
| 2245. | 4-((2-(6-methylpyridin-3-yl)-1-(2,2,2-trifluoroethyl)-1H-indol-4-yl)amino)tetrahydro- |
| | 2H-thiopyran 1,1-dioxide |
| 2246. | 4-((2-(6-(dimethylamino)pyridin-3-yl)-1-(2,2,2-trifluoroethyl)-1H-indol-4- |
| | yl)amino)tetrahydro-2H-thiopyran 1,1-dioxide |
| 2247. | 4-((2-(quinolin-4-yl)-1-(2,2,2-trifluoroethyl)-1H-indol-4-yl)amino)tetrahydro-2H- |
| | thiopyran 1,1-dioxide |
| 2248. | 4-((2-(2-fluoropyridin-3-yl)-1-(2,2,2-trifluoroethyl)-1H-indol-4-yl)amino)tetrahydro- |
| | 2H-thiopyran 1,1-dioxide |
| 2249. | 1-(4-((2-(5-aminopyridin-3-yl)-1-(2,2,2-trifluoroethyl)-1H-indol-4- |
| | yl)amino)piperidin-1-yl)-3-methoxypropan-2-ol |
| 2250. | 2-(2-amino-6-phenylpyridin-4-yl)-N-(1-methylpiperidin-4-yl)-1-(2,2,2-trifluoroethyl)- |
| | 1H-indol-4-amine |
| 2251. | 5-(4-((1,1-dioxidotetrahydro-2H-thiopyran-4-yl)amino)-1-(2,2,2-trifluoroethyl)-1H- |
| | indol-2-yl)-N-methylpicolinamide |
| 2252. | 2-(2-amino-6-phenylpyrimidin-4-yl)-N-(1-methylpiperidin-4-yl)-1-(2,2,2- |
| | trifluoroethyl)-1H-indol-4-amine |
| 2253. | 2-(2-amino-6-(cyclohex-1-en-1-yl)pyrimidin-4-yl)-N-(1-methylpiperidin-4-yl)-1- |
| | (2,2,2-trifluoroethyl)-1H-indol-4-amine |
| 2254. | 2-(2-amino-6-cyclohexylpyrimidin-4-yl)-N-(1-methylpiperidin-4-yl)-1-(2,2,2- |
| | trifluoroethyl)-1H-indol-4-amine |
| 2255. | 2-(2-(methylamino)pyrimidin-4-yl)-N-(1-methylpiperidin-4-yl)-1-(2,2,2- |
| | trifluoroethyl)-1H-indol-4-amine |
| 2256. | 2-(2-aminopyrimidin-4-yl)-N-(1-methylpiperidin-4-yl)-1-(2,2,2-trifluoroethyl)-1H- |
| | indol-4-amine |
| 2257. | 4-{[2-(1-methyl-1H-pyrazol-3-yl)-1-(2,2,2-trifluoroethyl)-1H-indol-4-yl]amino}-1λ⁶- |
| | thiane-1,1-dione |
| 2258. | 4-({2-[1-(2-hydroxyethyl)-1H-pyrazol-4-yl]-1-(2,2,2-trifluoroethyl)-1H-indol-4- |
| | yl}amino)-1λ⁶-thiane-1,1-dione |
| 2259. | 4-[(2-{1-[(pyridin-3-yl)methyl]-1H-pyrazol-4-yl}-1-(2,2,2-trifluoroethyl)-1H-indol-4- |
| | yl)amino]-1λ⁶-thiane-1,1-dione |
| 2260. | 4-((2-(1-(2-methoxyethyl)-1H-pyrazol-4-yl)-1-(2,2,2-trifluoroethyl)-1H-indol-4- |
| | yl)amino)tetrahydro-2H-thiopyran 1,1-dioxide |
| 2261. | 4-[(2-{1-[(pyridin-4-yl)methyl]-1H-pyrazol-4-yl}-1-(2,2,2-trifluoroethyl)-1H-indol-4- |
| | yl)amino]-1λ⁶-thiane-1,1-dione |
| 2262. | 2-(5-amino-1-phenyl-1H-pyrazol-3-yl)-N-(1-methylpiperidin-4-yl)-1-(2,2,2- |
| | trifluoroethyl)-1H-indol-4-amine |
| 2263. | 2-{5-[amino(phenyl)methyl]-1H-pyrazol-3-yl}-N-(1-methylpiperidin-4-yl)-1-(2,2,2- |
| | trifluoroethyl)-1H-indol-4-amine |
| 2264. | 2-(5-amino-1H-pyrazol-3-yl)-N-(1-methylpiperidin-4-yl)-1-(2,2,2-trifluoroethyl)-1H- |
| | indol-4-amine |
| 2265. | 2-(5-{[(4-methanesulfonylphenyl)amino]methyl}-1H-pyrazol-3-yl)-N-(1- |
| | methylpiperidin-4-yl)-1-(2,2,2-trifluoroethyl)-1H-indol-4-amine |
| 2266. | 2-[5-(methylamino)-1H-pyrazol-3-yl]-N-(1-methylpiperidin-4-yl)-1-(2,2,2- |
| | trifluoroethyl)-1H-indol-4-amine |
| 2267. | 2-(5-{[(4-methanesulfonylphenyl)amino]methyl}-1,3,4-thiadiazol-2-yl)-N-(1- |
| | methylpiperidin-4-yl)-1-(2,2,2-trifluoroethyl)-1H-indol-4-amine |
| 2268. | N-{[5-(4-{[(3R,4S)-3-fluoro-1-methylpiperidin-4-yl]amino}-1-(2,2,2-trifluoroethyl)- |
| | 1H-indol-2-yl)-1,3,4-thiadiazol-2-yl]methyl}cyclopropanecarboxamide |
| 2269. | N-{[5-(4-{[(3R,4S)-3-fluoro-1-methylpiperidin-4-yl]amino}-1-(2,2,2-trifluoroethyl)- |
| | 1H-indol-2-yl)-1,3,4-thiadiazol-2-yl]methyl}benzamide |
| 2270. | (+/−)-N-[(3R,4S)-3-fluoro-1-methylpiperidin-4-yl]-2-(5-{[(4-methanesulfonyl-2- |
| | methoxyphenyl)amino]methyl}-1,3,4-thiadiazol-2-yl)-1-(2,2,2-trifluoroethyl)-1H- |
| | indol-4-amine |
| 2271. | (+/−)-N-[(3R,4R)-3-fluoro-1-methylpiperidin-4-yl]-2-(5-{[(4-methanesulfonyl-2- |
| | methoxyphenyl)amino]methyl}-1,3,4-thiadiazol-2-yl)-1-(2,2,2-trifluoroethyl)-1H- |
| | indol-4-amine |
| 2272. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,3,4-thiadiazol-2-yl]methyl}cyclopentanecarboxamide |
| 2273. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,3,4-thiadiazol-2-yl]methyl}thiophene-2-carboxamide |
| 2274. | 1-fluoro-N-[(5-{4-[(1-methylpiperidin-4-yl)amino]-1-(2,2,2-trifluoroethyl)-1H-indol- |
| | 2-yl}-1,3,4-thiadiazol-2-yl)methyl]cyclopropane-1-carboxamide |
| 2275. | (+/−)-2,2-difluoro-N-[(5-{4-[(1-methylpiperidin-4-yl)amino]-1-(2,2,2-trifluoroethyl)- |
| | 1H-indol-2-yl}-1,3,4-thiadiazol-2-yl)methyl]cyclopropane-1-carboxamide |
| 2276. | (+/−)-(1R,2S)-2-methyl-N-[(5-{4-[(1-methylpiperidin-4-yl)amino]-1-(2,2,2- |
| | trifluoroethyl)-1H-indol-2-yl}-1,3,4-thiadiazol-2-yl)methyl]cyclopropane-1- |
| | carboxamide |
| 2277. | (+/−)-(1R,2R)-2-methyl-N-[(5-{4-[(1-methylpiperidin-4-yl)amino]-1-(2,2,2- |
| | trifluoroethyl)-1H-indol-2-yl}-1,3,4-thiadiazol-2-yl)methyl]cyclopropane-1- |
| | carboxamide |
| 2278. | N-[(5-{4-[(1-methylpiperidin-4-yl)amino]-1-(2,2,2-trifluoroethyl)-1H-indol-2-yl}- |
| | 1,3,4-thiadiazol-2-yl)methyl]cyclopropanecarboxamide |
| 2279. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,3,4-thiadiazol-2-yl]methyl}oxetane-3-carboxamide |
| 2280. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,3,4-thiadiazol-2-yl]methyl}cyclobutanecarboxamide |
| 2281. | (+/−)-methyl N-{[5-(4-{[(3R,4S)-3-fluoro-1-methylpiperidin-4-yl]amino}-1-(2,2,2- |
| | trifluoroethyl)-1H-indol-2-yl)-1,3,4-thiadiazol-2-yl]methyl}carbamate |
| 2282. | methyl 4-{[(5-{4-[(1-methylpiperidin-4-yl)amino]-1-(2,2,2-trifluoroethyl)-1H-indol-2- |
| | yl}-1,3,4-thiadiazol-2-yl)methyl]carbamoyl}benzoate |
| 2283. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,3,4-thiadiazol-2-yl]methyl}-1-methylpiperidine-4- |
| | carboxamide |
| 2284. | N-[(5-{4-[(1-methylpiperidin-4-yl)amino]-1-(2,2,2-trifluoroethyl)-1H-indol-2-yl}- |
| | 1,3,4-thiadiazol-2-yl)methyl]pyridine-2-carboxamide |
| 2285. | N-[(5-{4-[(1-methylpiperidin-4-yl)amino]-1-(2,2,2-trifluoroethyl)-1H-indol-2-yl}- |
| | 1,3,4-thiadiazol-2-yl)methyl]pyridine-3-carboxamide |
| 2286. | (+/−)-N-[(3R,4S)-3-fluoro-1-methylpiperidin-4-yl]-2-{5-[(methylamino)methyl]-1,3,4- |
| | thiadiazol-2-yl}-1-(2,2,2-trifluoroethyl)-1H-indol-4-amine |
| 2287. | (+/−)-benzyl N-{[5-(4-{[(3R,4S)-3-fluoro-1-methylpiperidin-4-yl]amino}-1-(2,2,2- |
| | trifluoroethyl)-1H-indol-2-yl)-1,3,4-thiadiazol-2-yl]methyl}carbamate |
| 2288. | N-[(5-{4-[(1-methylpiperidin-4-yl)amino]-1-(2,2,2-trifluoroethyl)-1H-indol-2-yl}- |
| | 1,3,4-thiadiazol-2-yl)methyl]-4-[(morpholin-4-yl)methyl]benzamide |
| 2289. | N-[(5-{4-[(1-methylpiperidin-4-yl)amino]-1-(2,2,2-trifluoroethyl)-1H-indol-2-yl}- |
| | 1,3,4-thiadiazol-2-yl)methyl]-3-[(morpholin-4-yl)methyl]benzamide |
| 2290. | N-{[5-(4-{[(3S,4R)-3-fluoro-1-methylpiperidin-4-yl]amino}-1-(2,2,2-trifluoroethyl)- |
| | 1H-indol-2-yl)-1,3,4-thiadiazol-2-yl]methyl}cyclopropanecarboxamide |
| 2291. | N-{[5-(4-{[(3R,4S)-3-fluoro-1-methylpiperidin-4-yl]amino}-1-(2,2,2-trifluoroethyl)- |
| | 1H-indol-2-yl)-1,3,4-thiadiazol-2-yl]methyl}cyclopropanecarboxamide |
| 2292. | N-[(5-{4-[(1-methylpiperidin-4-yl)amino]-1-(2,2,2-trifluoroethyl)-1H-indol-2-yl}- |
| | 1,3,4-thiadiazol-2-yl)methyl]pyridine-4-carboxamide |
| 2293. | 2-fluoro-N-[(5-{4-[(1-methylpiperidin-4-yl)amino]-1-(2,2,2-trifluoroethyl)-1H-indol- |
| | 2-yl}-1,3,4-thiadiazol-2-yl)methyl]benzamide |
| 2294. | 3-fluoro-N-[(5-{4-[(1-methylpiperidin-4-yl)amino]-1-(2,2,2-trifluoroethyl)-1H-indol- |
| | 2-yl}-1,3,4-thiadiazol-2-yl)methyl]benzamide |
| 2295. | 4-fluoro-N-[(5-{4-[(1-methylpiperidin-4-yl)amino]-1-(2,2,2-trifluoroethyl)-1H-indol- |
| | 2-yl}-1,3,4-thiadiazol-2-yl)methyl]benzamide |
| 2296. | (+/−)-(1S,2S)-N-[(5-{4-[(1-methylpiperidin-4-yl)amino]-1-(2,2,2-trifluoroethyl)-1H- |
| | indol-2-yl}-1,3,4-thiadiazol-2-yl)methyl]-2-phenylcyclopropane-1-carboxamide |
| 2297. | 4-{[(5-{4-[(1-methylpiperidin-4-yl)amino]-1-(2,2,2-trifluoroethyl)-1H-indol-2-yl}- |
| | 1,3,4-thiadiazol-2-yl)methyl]carbamoyl}benzoic acid |
| 2298. | N-[(5-{4-[(1-methylpiperidin-4-yl)amino]-1-(2,2,2-trifluoroethyl)-1H-indol-2-yl}- |
| | 1,3,4-thiadiazol-2-yl)methyl]-1H-indazole-5-carboxamide |
| 2299. | 3-methyl-1-[(5-{4-[(1-methylpiperidin-4-yl)amino]-1-(2,2,2-trifluoroethyl)-1H-indol- |
| | 2-yl}-1,3,4-thiadiazol-2-yl)methyl]urea |
| 2300. | 2-methyl-N-[(5-{4-[(1-methylpiperidin-4-yl)amino]-1-(2,2,2-trifluoroethyl)-1H-indol- |
| | 2-yl}-1,3,4-thiadiazol-2-yl)methyl]propanamide |
| 2301. | N-[(5-{4-[(1-methylpiperidin-4-yl)amino]-1-(2,2,2-trifluoroethyl)-1H-indol-2-yl}- |
| | 1,3,4-thiadiazol-2-yl)methyl]acetamide |
| 2302. | N-[(5-{4-[(1-methylpiperidin-4-yl)amino]-1-(2,2,2-trifluoroethyl)-1H-indol-2-yl}- |
| | 1,3,4-thiadiazol-2-yl)methyl]-2-phenylacetamide |
| 2303. | 2-methoxy-N-[(5-{4-[(1-methylpiperidin-4-yl)amino]-1-(2,2,2-trifluoroethyl)-1H- |
| | indol-2-yl}-1,3,4-thiadiazol-2-yl)methyl]acetamide |
| 2304. | N-[(3R,4S)-3-fluoro-1-methylpiperidin-4-yl]-2-(5-{[(4-methanesulfonyl-2- |
| | methoxyphenyl)amino]methyl}-1,3,4-thiadiazol-2-yl)-1-(2,2,2-trifluoroethyl)-1H- |
| | indol-4-amine |
| 2305. | N-[(3S,4R)-3-fluoro-1-methylpiperidin-4-yl]-2-(5-{[(4-methanesulfonyl-2- |
| | methoxyphenyl)amino]methyl}-1,3,4-thiadiazol-2-yl)-1-(2,2,2-trifluoroethyl)-1H- |
| | indol-4-amine |
| 2306. | 4-methoxy-N-[(5-{4-[(1-methylpiperidin-4-yl)amino]-1-(2,2,2-trifluoroethyl)-1H- |
| | indol-2-yl}-1,3,4-thiadiazol-2-yl)methyl]benzamide |
| 2307. | 3-methoxy-N-[(5-{4-[(1-methylpiperidin-4-yl)amino]-1-(2,2,2-trifluoroethyl)-1H- |
| | indol-2-yl}-1,3,4-thiadiazol-2-yl)methyl]benzamide |
| 2308. | N-[(5-{4-[(1-methylpiperidin-4-yl)amino]-1-(2,2,2-trifluoroethyl)-1H-indol-2-yl}- |
| | 1,3,4-thiadiazol-2-yl)methyl]butanamide |
| 2309. | 2-methoxy-N-[(5-{4-[(1-methylpiperidin-4-yl)amino]-1-(2,2,2-trifluoroethyl)-1H- |
| | indol-2-yl}-1,3,4-thiadiazol-2-yl)methyl]benzamide |
| 2310. | 2-[5-(aminomethyl)-1,3,4-thiadiazol-2-yl]-N-(1-methylpiperidin-4-yl)-1-(2,2,2- |
| | trifluoroethyl)-1H-indol-4-amine |
| 2311. | 3,3-dimethyl-1-[(5-{4-[(1-methylpiperidin-4-yl)amino]-1-(2,2,2-trifluoroethyl)-1H- |
| | indol-2-yl}-1,3,4-thiadiazol-2-yl)methyl]urea |
| 2312. | N-[(5-{4-[(1-methylpiperidin-4-yl)amino]-1-(2,2,2-trifluoroethyl)-1H-indol-2-yl}- |
| | 1,3,4-thiadiazol-2-yl)methyl]-1H-indazole-6-carboxamide |
| 2313. | benzyl N-{[5-(4-{[(3S,4R)-3-fluoropiperidin-4-yl]amino}-1-(2,2,2-trifluoroethyl)-1H- |
| | indol-2-yl)-1,3,4-thiadiazol-2-yl]methyl}carbamate |
| 2314. | 1-methyl-N-[(5-{4-[(1-methylpiperidin-4-yl)amino]-1-(2,2,2-trifluoroethyl)-1H-indol- |
| | 2-yl}-1,3,4-thiadiazol-2-yl)methyl]cyclopropane-1-carboxamide |
| 2315. | N-[(5-{4-[(1-methylpiperidin-4-yl)amino]-1-(2,2,2-trifluoroethyl)-1H-indol-2-yl}- |
| | 1,3,4-thiadiazol-2-yl)methyl]-1H-pyrazole-4-carboxamide |
| 2316. | N-[(5-{4-[(1-methylpiperidin-4-yl)amino]-1-(2,2,2-trifluoroethyl)-1H-indol-2-yl}- |
| | 1,3,4-thiadiazol-2-yl)methyl]-1H-pyrazole-5-carboxamide |
| 2317. | 1-ethyl-N-[(5-{4-[(1-methylpiperidin-4-yl)amino]-1-(2,2,2-trifluoroethyl)-1H-indol-2- |
| | yl}-1,3,4-thiadiazol-2-yl)methyl]cyclopropane-1-carboxamide |
| 2318. | (+/−)-methyl (1R,2R)-2-{[(5-{4-[(1-methylpiperidin-4-yl)amino]-1-(2,2,2- |
| | trifluoroethyl)-1H-indol-2-yl}-1,3,4-thiadiazol-2-yl)methyl]carbamoyl}cyclopropane- |
| | 1-carboxylate |
| 2319. | (+/−)-(1R,2R)-2-{[(5-{4-[(1-methylpiperidin-4-yl)amino]-1-(2,2,2-trifluoroethyl)-1H- |
| | indol-2-yl}-1,3,4-thiadiazol-2-yl)methyl]carbamoyl}cyclopropane-1-carboxylic acid |
| 2320. | 1-methyl-N-[(5-{4-[(1-methylpiperidin-4-yl)amino]-1-(2,2,2-trifluoroethyl)-1H-indol- |
| | 2-yl}-1,3,4-thiadiazol-2-yl)methyl]-1H-pyrrole-3-carboxamide |
| 2321. | N-[(5-{4-[(1-methylpiperidin-4-yl)amino]-1-(2,2,2-trifluoroethyl)-1H-indol-2-yl}- |
| | 1,3,4-thiadiazol-2-yl)methyl]-1H-pyrrole-3-carboxamide |
| 2322. | N-[(5-{4-[(1-methylpiperidin-4-yl)amino]-1-(2,2,2-trifluoroethyl)-1H-indol-2-yl}- |
| | 1,3,4-thiadiazol-2-yl)methyl]-2-(thiophen-2-yl)cyclopropane-1-carboxamide |
| 2323. | N-(1-methylpiperidin-4-yl)-2-(5-{[(pyrrolidin-3-yl)amino]methyl}-1,3,4-thiadiazol-2- |
| | yl)-1-(2,2,2-trifluoroethyl)-1H-indol-4-amine |
| 2324. | (+/−)-(1R,2S)-2-{[(5-{4-[(1-methylpiperidin-4-yl)amino]-1-(2,2,2-trifluoroethyl)-1H- |
| | indol-2-yl}-1,3,4-thiadiazol-2-yl)methyl]carbamoyl}cyclopropane-1-carboxylic acid |
| 2325. | N-(1-methylpiperidin-4-yl)-2-(5-{[(1H-pyrazol-4-yl)amino]methyl}-1,3,4-thiadiazol- |
| | 2-yl)-1-(2,2,2-trifluoroethyl)-1H-indol-4-amine |
| 2326. | N-((5-(4-(((3S,4R)-3-fluoro-1-methylpiperidin-4-yl)amino)-1-(2,2,2-trifluoroethyl)- |
| | 1H-indol-2-yl)-1,3,4-thiadiazol-2-yl)methyl)cyclopentanecarboxamide |
| 2327. | N-((3S,4R)-3-fluoro-1-methylpiperidin-4-yl)-2-(5-(((2-methoxy-4- |
| | (methylsulfonyl)phenyl)amino)methyl)-1,3,4-thiadiazol-2-yl)-1-(2,2,2-trifluoroethyl)- |
| | 1H-indol-4-amine |
| 2328. | N-((5-(4-(((3S,4R)-3-fluoro-1-methylpiperidin-4-yl)amino)-1-(2,2,2-trifluoroethyl)- |
| | 1H-indol-2-yl)-1,3,4-thiadiazol-2-yl)methyl)benzamide |
| 2329. | N-((5-(4-(((3S,4R)-3-fluoro-1-methylpiperidin-4-yl)amino)-1-(2,2,2-trifluoroethyl)- |
| | 1H-indol-2-yl)-1,3,4-thiadiazol-2-yl)methyl)cyclopropanecarboxamide |
| 2330. | 2-(5-((dimethylamino)methyl)-1,3,4-thiadiazol-2-yl)-N-((3S,4R)-3-fluoro-1- |
| | methylpiperidin-4-yl)-1-(2,2,2-trifluoroethyl)-1H-indol-4-amine |
| 2331. | N-((3S,4S)-3-fluoro-1-methylpiperidin-4-yl)-2-(5-(((2-methoxy-4- |
| | (methylsulfonyl)phenyl)amino)methyl)-1,3,4-thiadiazol-2-yl)-1-(2,2,2-trifluoroethyl)- |
| | 1H-indol-4-amine |
| 2332. | N-{[2-(4-{[(3S,4R)-3-fluoro-1-methylpiperidin-4-yl]amino}-1-(2,2,2-trifluoroethyl)- |
| | 1H-indol-2-yl)-1,3-thiazol-4-yl]methyl}cyclopropanecarboxamide |
| 2333. | N-{[2-(4-{[(3S,4R)-3-fluoro-1-methylpiperidin-4-yl]amino}-1-(2,2,2-trifluoroethyl)- |
| | 1H-indol-2-yl)-1,3-thiazol-4-yl]methyl}benzamide |
| 2334. | (+/−)-N-{[2-(4-{[(3R,4S)-3-fluoro-1-methylpiperidin-4-yl] amino}-1-(2,2,2- |
| | trifluoroethyl)-1H-indol-2-yl)-1,3-thiazol-5-yl]methyl}benzamide |
| 2335. | N-[(2-{4-[(1-methylpiperidin-4-yl)amino]-1-(2,2,2-trifluoroethyl)-1H-indol-2-yl}-1,3- |
| | thiazol-5-yl)methyl]benzamide |
| 2336. | (+/−)-N-[(3R,4S)-3-fluoro-1-methylpiperidin-4-yl]-2-(1,3-thiazol-2-yl)-1-(2,2,2- |
| | trifluoroethyl)-1H-indol-4-amine |
| 2337. | N-[(2-{4-[(1-methylpiperidin-4-yl)amino]-1-(2,2,2-trifluoroethyl)-1H-indol-2-yl}-1,3- |
| | thiazol-5-yl)methyl]cyclopropanecarboxamide |
| 2338. | (+/−)-N-{[2-(4-{[(3R,4S)-3-fluoro-1-methylpiperidin-4-yl]amino}-1-(2,2,2- |
| | trifluoroethyl)-1H-indol-2-yl)-1,3-thiazol-5-yl]methyl}cyclopropanecarboxamide |
| 2339. | (+/−)-2-[5-(aminomethyl)-1,3-thiazol-2-yl]-N-[(3R,4S)-3-fluoro-1-methylpiperidin-4- |
| | yl]-1-(2,2,2-trifluoroethyl)-1H-indol-4-amine |
| 2340. | 2-(4-(aminomethyl)thiazol-2-yl)-N-((3S,4R)-3-fluoro-1-methylpiperidin-4-yl)-1- |
| | (2,2,2-trifluoroethyl)-1H-indol-4-amine |
| 2341. | (+/−)-N-[(3R,4S)-3-fluoro-1-methylpiperidin-4-yl]-2-{5-[(phenylamino)methyl]-1,3- |
| | thiazol-2-yl}-1-(2,2,2-trifluoroethyl)-1H-indol-4-amine |
| 2342. | N-[(3S,4R)-3-fluoro-1-methylpiperidin-4-yl]-2-(5-{[(4-methanesulfonyl-2- |
| | methoxyphenyl)amino]methyl}-1,3-thiazol-2-yl)-1-(2,2,2-trifluoroethyl)-1H-indol-4- |
| | amine |
| 2343. | 2-(5-{[(4-methanesulfonylphenyl)amino]methyl}thiophen-2-yl)-N-(1- |
| | methylpiperidin-4-yl)-1-(2,2,2-trifluoroethyl)-1H-indol-4-amine |
| 2344. | (+/−)-N-[(3R,4S)-3-fluoro-1-methylpiperidin-4-yl]-2-(5-{[(4-methanesulfonyl-2- |
| | methoxyphenyl)amino]methyl}thiophen-2-yl)-1-(2,2,2-trifluoroethyl)-1H-indol-4- |
| | amine |
| 2345. | (+/−)-N-{[5-(4-{[(3R,4S)-3-fluoro-1-methylpiperidin-4-yl]amino}-1-(2,2,2- |
| | trifluoroethyl)-1H-indol-2-yl)thiophen-2-yl]methyl}benzamide |
| 2346. | (+/−)-N-{[5-(4-{[(3R,4S)-3-fluoro-1-methylpiperidin-4-yl]amino}-1-(2,2,2- |
| | trifluoroethyl)-1H-indol-2-yl)thiophen-2-yl]methyl}cyclopropanecarboxamide |
| 2347. | N-(1-methylpiperidin-4-yl)-2-(5-(((4-(methylsulfonyl)phenyl)amino)methyl)thiophen- |
| | 2-yl)-1-(2,2,2-trifluoroethyl)-1H-indol-4-amine |
| 2348. | N-((5-(4-(((3S,4R)-3-fluoro-1-methylpiperidin-4-yl)amino)-1-(2,2,2-trifluoroethyl)- |
| | 1H-indol-2-yl)thiophen-2-yl)methyl)benzamide |
| 2349. | N-((3S,4R)-3-fluoro-1-methylpiperidin-4-yl)-2-(5-(((2-methoxy-4- |
| | (methylsulfonyl)phenyl)amino)methyl)thiophen-2-yl)-1-(2,2,2-trifluoroethyl)-1H- |
| | indol-4-amine |
| 2350. | 2-(5-(amino(cyclohexyl)methyl)-1,3,4-oxadiazol-2-yl)-N-(1-methylpiperidin-4-yl)-1- |
| | (2,2,2-trifluoroethyl)-1H-indol-4-amine |
| 2351. | N-(1-methylpiperidin-4-yl)-2-{5-[(phenylamino)methyl]-1,3,4-oxadiazol-2-yl}-1- |
| | (2,2,2-trifluoroethyl)-1H-indol-4-amine |
| 2352. | 2-(5-(amino(tetrahydro-2H-pyran-4-yl)methyl)-1,3,4-oxadiazol-2-yl)-N-(1- |
| | methylpiperidin-4-yl)-1-(2,2,2-trifluoroethyl)-1H-indol-4-amine |
| 2353. | N-[(5-{4-[(1-methylpiperidin-4-yl)amino]-1-(2,2,2-trifluoroethyl)-1H-indol-2-yl}- |
| | 1,3,4-oxadiazol-2-yl)methyl]cyclopropanecarboxamide |
| 2354. | 2-(5-{[(4-methanesulfonylphenyl)amino]methyl}-1,3,4-oxadiazol-2-yl)-N-(1- |
| | methylpiperidin-4-yl)-1-(2,2,2-trifluoroethyl)-1H-indol-4-amine |
| 2355. | N-(1-methylpiperidin-4-yl)-2-(5-{[(1H-pyrazol-4-yl)amino]methyl}-1,3,4-oxadiazol- |
| | 2-yl)-1-(2,2,2-trifluoroethyl)-1H-indol-4-amine |
| 2356. | 2-(5-(aminomethyl)-1,3,4-oxadiazol-2-yl)-N-(1-methylpiperidin-4-yl)-1-(2,2,2- |
| | trifluoroethyl)-1H-indol-4-amine |
| 2357. | (+/−)-N-[(3R,4S)-3-fluoro-1-methylpiperidin-4-yl]-2-(5-{[(4-methanesulfonyl-2- |
| | methoxyphenyl)amino]methyl}-1,3,4-oxadiazol-2-yl)-1-(2,2,2-trifluoroethyl)-1H- |
| | indol-4-amine |
| 2358. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,3,4-oxadiazol-2-yl]methyl}-2-methoxybenzamide |
| 2359. | (+/−)-2-[5-(aminomethyl)-1,3,4-oxadiazol-2-yl]-N-[(3R,4S)-3-fluoro-1- |
| | methylpiperidin-4-yl]-1-(2,2,2-trifluoroethyl)-1H-indol-4-amine |
| 2360. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,3,4-oxadiazol-2-yl]methyl}-4-methoxybenzamide |
| 2361. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,3,4-oxadiazol-2-yl]methyl}benzamide |
| 2362. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,3,4-oxadiazol-2-yl]methyl}-3-methoxybenzamide |
| 2363. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,3,4-oxadiazol-2-yl]methyl}thiophene-2-carboxamide |
| 2364. | (+/−)-2-(5-{[(cyclopropylmethyl)amino]methyl}-1,3,4-oxadiazol-2-yl)-N-[(3R,4S)-3- |
| | fluoro-1-methylpiperidin-4-yl]-1-(2,2,2-trifluoroethyl)-1H-indol-4-amine |
| 2365. | (+/−)-N-[(3R,4S)-3-fluoro-1-methylpiperidin-4-yl]-2-(5-{[(3- |
| | methanesulfonylphenyl)amino]methyl}-1,3,4-oxadiazol-2-yl)-1-(2,2,2-trifluoroethyl)- |
| | 1H-indol-4-amine |
| 2366. | (+/−)-N-[(3R,4R)-3-fluoro-1-methylpiperidin-4-yl]-2-(5-{[(4-methanesulfonyl-2- |
| | methoxyphenyl)amino]methyl}-1,3,4-oxadiazol-2-yl)-1-(2,2,2-trifluoroethyl)-1H- |
| | indol-4-amine |
| 2367. | (+/−)-2-(5-((bis(cyclopropylmethyl)amino)methyl)-1,3,4-oxadiazol-2-yl)-N-((3R,4S)- |
| | 3-fluoro-1-methylpiperidin-4-yl)-1-(2,2,2-trifluoroethyl)-1H-indol-4-amine |
| 2368. | (+/−)-N-((3S,4R)-3-fluoro-1-methylpiperidin-4-yl)-2-(5-(((3- |
| | (methylsulfonyl)phenyl)amino)methyl)-1,3,4-oxadiazol-2-yl)-1-(2,2,2-trifluoroethyl)- |
| | 1H-indol-4-amine |
| 2369. | (+/−)-N-((3S,4R)-3-fluoro-1-methylpiperidin-4-yl)-2-(5-(((2-methoxy-4- |
| | (methylsulfonyl)phenyl)amino)methyl)-1,3,4-oxadiazol-2-yl)-1-(2,2,2-trifluoroethyl)- |
| | 1H-indol-4-amine |
| 2370. | (+/−)-2-(5-(aminomethyl)-1,3,4-oxadiazol-2-yl)-N-((3S,4R)-3-fluoro-1- |
| | methylpiperidin-4-yl)-1-(2,2,2-trifluoroethyl)-1H-indol-4-amine |
| 2371. | (+/−)-N-((3S,4S)-3-fluoro-1-methylpiperidin-4-yl)-2-(5-(((2-methoxy-4- |
| | (methylsulfonyl)phenyl)amino)methyl)-1,3,4-oxadiazol-2-yl)-1-(2,2,2-trifluoroethyl)- |
| | 1H-indol-4-amine |
| 2372. | N-((5-(4-(((3S,4R)-3-fluoro-1-methylpiperidin-4-yl)amino)-1-(2,2,2-trifluoroethyl)- |
| | 1H-indol-2-yl)-1,3,4-oxadiazol-2-yl)methyl)benzamide |
| 2373. | (+/−)-N-((5-(4-(((3S,4R)-3-fluoro-1-methylpiperidin-4-yl)amino)-1-(2,2,2- |
| | trifluoroethyl)-1H-indol-2-yl)-1,3,4-oxadiazol-2-yl)methyl)thiophene-2-carboxamide |
| 2374. | (+/−)-N-((5-(4-(((3S,4R)-3-fluoro-1-methylpiperidin-4-yl)amino)-1-(2,2,2- |
| | trifluoroethyl)-1H-indol-2-yl)-1,3,4-oxadiazol-2-yl)methyl)-2-methoxybenzamide |
| 2375. | (+/−)-N-((5-(4-(((3S,4R)-3-fluoro-1-methylpiperidin-4-yl)amino)-1-(2,2,2- |
| | trifluoroethyl)-1H-indol-2-yl)-1,3,4-oxadiazol-2-yl)methyl)-3-methoxybenzamide |
| 2376. | (+/−)-N-((5-(4-(((3S,4R)-3-fluoro-1-methylpiperidin-4-yl)amino)-1-(2,2,2- |
| | trifluoroethyl)-1H-indol-2-yl)-1,3,4-oxadiazol-2-yl)methyl)-4-methoxybenzamide |
| 2377. | (+/−)-2-(5-(((cyclopropylmethyl)amino)methyl)-1,3,4-oxadiazol-2-yl)-N-((3S,4R)-3- |
| | fluoro-1-methylpiperidin-4-yl)-1-(2,2,2-trifluoroethyl)-1H-indol-4-amine |
| 2378. | (+/−)-N-((3R,4R)-3-fluoro-1-methylpiperidin-4-yl)-2-(5-(((2-methoxy-4- |
| | (methylsulfonyl)phenyl)amino)methyl)-1,3,4-oxadiazol-2-yl)-1-(2,2,2-trifluoroethyl)- |
| | 1H-indol-4-amine |
| 2379. | 2-(3-{[(4-methanesulfonylphenyl)amino]methyl}-1,2,4-oxadiazol-5-yl)-N-(1- |
| | methylpiperidin-4-yl)-1-(2,2,2-trifluoroethyl)-1H-indol-4-amine |
| 2380. | N-[(5-{4-[(1-methylpiperidin-4-yl)amino]-1-(2,2,2-trifluoroethyl)-1H-indol-2-yl}- |
| | 1,2,4-oxadiazol-3-yl)methyl]cyclopropanecarboxamide |
| 2381. | N-[(5-{4-[(1-methylpiperidin-4-yl)amino]-1-(2,2,2-trifluoroethyl)-1H-indol-2-yl}- |
| | 1,2,4-oxadiazol-3-yl)methyl]benzamide |
| 2382. | N-[(5-{4-[(1-methylpiperidin-4-yl)amino]-1-(2,2,2-trifluoroethyl)-1H-indol-2-yl}- |
| | 1,2,4-oxadiazol-3-yl)methyl]thiophene-2-carboxamide |
| 2383. | 1-methyl-N-[(5-{4-[(1-methylpiperidin-4-yl)amino]-1-(2,2,2-trifluoroethyl)-1H-indol- |
| | 2-yl}-1,2,4-oxadiazol-3-yl)methyl]-1H-pyrazole-4-carboxamide |
| 2384. | 1-methyl-N-[(5-{4-[(1-methylpiperidin-4-yl)amino]-1-(2,2,2-trifluoroethyl)-1H-indol- |
| | 2-yl}-1,2,4-oxadiazol-3-yl)methyl]-1H-pyrazole-3-carboxamide |
| 2385. | N-(1-methylpiperidin-4-yl)-2-{5-[(phenylamino)methyl]-1,2,4-oxadiazol-3-yl}-1- |
| | (2,2,2-trifluoroethyl)-1H-indol-4-amine |
| 2386. | N-[(3-{4-[(1-methylpiperidin-4-yl)amino]-1-(2,2,2-trifluoroethyl)-1H-indol-2-yl}- |
| | 1,2,4-oxadiazol-5-yl)methyl]thiophene-2-carboxamide |
| 2387. | N-[(3-{4-[(1-methylpiperidin-4-yl)amino]-1-(2,2,2-trifluoroethyl)-1H-indol-2-yl}- |
| | 1,2,4-oxadiazol-5-yl)methyl]benzamide |
| 2388. | (+/−)-(1S,2R)-2-fluoro-N-[(3-{4-[(1-methylpiperidin-4-yl)amino]-1-(2,2,2- |
| | trifluoroethyl)-1H-indol-2-yl}-1,2,4-oxadiazol-5-yl)methyl]cyclopropane-1- |
| | carboxamide |
| 2389. | N-[(3-{4-[(1-methylpiperidin-4-yl)amino]-1-(2,2,2-trifluoroethyl)-1H-indol-2-yl}- |
| | 1,2,4-oxadiazol-5-yl)methyl]cyclopropanecarboxamide |
| 2390. | (+/−)-(1S,2S)-2-fluoro-N-[(3-{4-[(1-methylpiperidin-4-yl)amino]-1-(2,2,2- |
| | trifluoroethyl)-1H-indol-2-yl}-1,2,4-oxadiazol-5-yl]methyl]cyclopropane-1- |
| | carboxamide |
| 2391. | 4-chloro-N-[(3-{4-[(1-methylpiperidin-4-yl)amino]-1-(2,2,2-trifluoroethyl)-1H-indol- |
| | 2-yl}-1,2,4-oxadiazol-5-yl)methyl]benzamide |
| 2392. | N-[(3-{4-[(1-methylpiperidin-4-yl)amino]-1-(2,2,2-trifluoroethyl)-1H-indol-2-yl}- |
| | 1,2,4-oxadiazol-5-yl)methyl]-1,3-thiazole-2-carboxamide |
| 2393. | 4-fluoro-N-[(3-{4-[(1-methylpiperidin-4-yl)amino]-1-(2,2,2-trifluoroethyl)-1H-indol- |
| | 2-yl}-1,2,4-oxadiazol-5-yl)methyl]benzamide |
| 2394. | 4-cyano-N-[(3-{4-[(1-methylpiperidin-4-yl)amino]-1-(2,2,2-trifluoroethyl)-1H-indol- |
| | 2-yl}-1,2,4-oxadiazol-5-yl)methyl]benzamide |
| 2395. | 1-methyl-N-[(3-{4-[(1-methylpiperidin-4-yl)amino]-1-(2,2,2-trifluoroethyl)-1H-indol- |
| | 2-yl}-1,2,4-oxadiazol-5-yl)methyl]-1H-pyrrole-3-carboxamide |
| 2396. | 3-[(3-{4-[(1-methylpiperidin-4-yl)amino]-1-(2,2,2-trifluoroethyl)-1H-indol-2-yl}- |
| | 1,2,4-oxadiazol-5-yl)methyl]-1-phenylurea |
| 2397. | 1-methyl-N-[(3-{4-[(1-methylpiperidin-4-yl)amino]-1-(2,2,2-trifluoroethyl)-1H-indol- |
| | 2-yl}-1,2,4-oxadiazol-5-yl)methyl]-1H-pyrazole-4-carboxamide |
| 2398. | 1-methyl-N-[(3-{4-[(1-methylpiperidin-4-yl)amino]-1-(2,2,2-trifluoroethyl)-1H-indol- |
| | 2-yl}-1,2,4-oxadiazol-5-yl)methyl]-1H-pyrazole-3-carboxamide |
| 2399. | (+/−)-(1R,2R)-N-[(3-{4-[(1-methylpiperidin-4-yl)amino]-1-(2,2,2-trifluoroethyl)-1H- |
| | indol-2-yl}-1,2,4-oxadiazol-5-yl)methyl]-2-phenylcyclopropane-1-carboxamide |
| 2400. | (+/−)-(1R,2R)-2-methyl-N-[(3-{4-[(1-methylpiperidin-4-yl)amino]-1-(2,2,2- |
| | trifluoroethyl)-1H-indol-2-yl}-1,2,4-oxadiazol-5-yl)methyl]cyclopropane-1-carboxamide |
| 2401. | (+/−)-(1R,2S)-2-methyl-N-[(3-{4-[(1-methylpiperidin-4-yl)amino]-1-(2,2,2- |
| | trifluoroethyl)-1H-indol-2-yl}-1,2,4-oxadiazol-5-yljmethyl]cyclopropane-1- |
| | carboxamide |
| 2402. | N-[(3-{4-[(oxan-4-yl)amino]-1-(2,2,2-trifluoroethyl)-1H-indol-2-yl}-1,2,4-oxadiazol- |
| | 5-yl)methyl]cyclopropanecarboxamide |
| 2403. | N-({3-[4-(benzylamino)-1-(2,2,2-trifluoroethyl)-1H-indol-2-yl]-1,2,4-oxadiazol-5- |
| | yl}methyl)cyclopropanecarboxamide |
| 2404. | N-[(3-{4-[(1-acetylpiperidin-4-yl)amino]-1-(2,2,2-trifluoroethyl)-1H-indol-2-yl}- |
| | 1,2,4-oxadiazol-5-yl)methyl]cyclopropanecarboxamide |
| 2405. | N-[(3-{4-[(1,1-dioxo-1λ⁶-thian-4-yl)amino]-1-(2,2,2-trifluoroethyl)-1H-indol-2-yl}- |
| | 1,2,4-oxadiazol-5-yl)methyl]cyclopropanecarboxamide |
| 2406. | N-[(3-{4-[(1-benzylpiperidin-4-yl)amino]-1-(2,2,2-trifluoroethyl)-1H-indol-2-yl}- |
| | 1,2,4-oxadiazol-5-yl)methyl]cyclopropanecarboxamide |
| 2407. | N-[(3-{4-[(1-cyclopropylpiperidin-4-yl)amino]-1-(2,2,2-trifluoroethyl)-1H-indol-2- |
| | yl}-1,2,4-oxadiazol-5-yl)methyl]cyclopropanecarboxamide |
| 2408. | N-[(3-{4-[(cyclopropylmethyl)amino]-1-(2,2,2-trifluoroethyl)-1H-indol-2-yl}-1,2,4- |
| | oxadiazol-5-yl)methyl]cyclopropanecarboxamide |
| 2409. | N-[(3-{4-[(cyclobutylmethyl)amino]-1-(2,2,2-trifluoroethyl)-1H-indol-2-yl}-1,2,4- |
| | oxadiazol-5-yl)methyl]cyclopropanecarboxamide |
| 2410. | (+/−)-N-[(3-{4-[(pyrrolidin-3-yl)amino]-1-(2,2,2-trifluoroethyl)-1H-indol-2-yl}-1,2,4- |
| | oxadiazol-5-yl)methyl]cyclopropanecarboxamide |
| 2411. | N-[(3-{4-[(1-ethylpiperidin-4-yl)amino]-1-(2,2,2-trifluoroethyl)-1H-indol-2-yl}-1,2,4- |
| | oxadiazol-5-yl)methyl]cyclopropanecarboxamide |
| 2412. | (+/−)-N-[(3-{4-[(1-methylpyrrolidin-3-yl)amino]-1-(2,2,2-trifluoroethyl)-1H-indol-2- |
| | yl}-1,2,4-oxadiazol-5-yl)methyl]cyclopropanecarboxamide |
| 2413. | N-{[3-(4-{[(273zetidine-3-yl)methyl]amino}-1-(2,2,2-trifluoroethyl)-1H-indol-2-yl)- |
| | 1,2,4-oxadiazol-5-yl]methyl}cyclopropanecarboxamide |
| 2414. | (+/−)-N-{[3-(4-{[(3R,4S)-3-fluoro-1-methylpiperidin-4-yl]amino}-1-(2,2,2- |
| | trifluoroethyl)-1H-indol-2-yl)-1,2,4-oxadiazol-5-yl]methyl}cyclopropanecarboxamide |
| 2415. | (+/−)-N-{[3-(4-{[(3R,4S)-3-fluoro-1-methylpiperidin-4-yl]amino}-1-(2,2,2- |
| | trifluoroethyl)-1H-indol-2-yl)-1,2,4-oxadiazol-5-yl]methyl}benzamide |
| 2416. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}cyclopropanecarboxamide |
| 2417. | N-{[3-(4-{[(3R,4S)-3-fluoro-1-methylpiperidin-4-yl]amino}-1-(2,2,2-trifluoroethyl)- |
| | 1H-indol-2-yl)-1,2,4-oxadiazol-5-yl]methyl}cyclopropanecarboxamide |
| 2418. | (+/−)-N-{[3-(4-{[(3R,4S)-3-fluoro-1-methylpiperidin-4-yl]amino}-1-(2,2,2- |
| | trifluoroethyl)-1H-indol-2-yl)-1,2,4-oxadiazol-5-yl]methyl}thiophene-2-carboxamide |
| 2419. | (+/−)-N-{[3-(4-{[(3R,4S)-3-fluoro-1-methylpiperidin-4-yl]amino}-1-(2,2,2- |
| | trifluoroethyl)-1H-indol-2-yl)-1,2,4-oxadiazol-5-yl]methyl}thiophene-3-carboxamide |
| 2420. | (+/−)-N-[(3R,4S)-3-fluoro-1-methylpiperidin-4-yl]-2-(5-{[(4- |
| | methanesulfonylphenyl)amino]methyl}-1,2,4-oxadiazol-3-yl)-1-(2,2,2-trifluoroethyl)- |
| | 1H-indol-4-amine |
| 2421. | (+/−)-N-{[3-(4-{[(3R,4S)-3-fluoro-1-methylpiperidin-4-yl]amino}-1-(2,2,2- |
| | trifluoroethyl)-1H-indol-2-yl)-1,2,4-oxadiazol-5-yl]methyl}-1,3-thiazole-5- |
| | carboxamide |
| 2422. | (+/−)-1-ethyl-N-{[3-(4-{[(3R,4S)-3-fluoro-1-methylpiperidin-4-yl]amino}-1-(2,2,2- |
| | trifluoroethyl)-1H-indol-2-yl)-1,2,4-oxadiazol-5-yl]methyl}-1H-pyrrole-3- |
| | carboxamide |
| 2423. | (+/−)-(1R,2R)-N-{[3-(4-{[(3R,4S)-3-fluoro-1-methylpiperidin-4-yl]amino}-1-(2,2,2- |
| | trifluoroethyl)-1H-indol-2-yl)-1,2,4-oxadiazol-5-yl]methyl}-2-phenylcyclopropane-1- |
| | carboxamide |
| 2424. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-methyl-1H-pyrrole-3-carboxamide |
| 2425. | N-{[3-(4-{[(3R,4S)-3-fluoro-1-methylpiperidin-4-yl]amino}-1-(2,2,2-trifluoroethyl)- |
| | 1H-indol-2-yl)-1,2,4-oxadiazol-5-yl]methyl}-1-methyl-1H-pyrrole-3-carboxamide |
| 2426. | (+/−)-N-[(3R,4S)-3-fluoro-1-methylpiperidin-4-yl]-2-(5-{[(4-methanesulfonyl-2- |
| | methoxyphenyl)amino]methyl}-1,2,4-oxadiazol-3-yl)-1-(2,2,2-trifluoroethyl)-1H- |
| | indol-4-amine |
| 2427. | (+/−)-1-tert-butyl-N-{[3-(4-{[(3R,4S)-3-fluoro-1-methylpiperidin-4-yl]amino}-1- |
| | (2,2,2-trifluoroethyl)-1H-indol-2-yl)-1,2,4-oxadiazol-5-yl]methyl}-1H-pyrrole-3- |
| | carboxamide |
| 2428. | (+/−)-(1R,2R)-N-{[3-(4-{[(3RS,4SR)-3-fluoro-1-methylpiperidin-4-yl]amino}-1- |
| | (2,2,2-trifluoroethyl)-1H-indol-2-yl)-1,2,4-oxadiazol-5-yl]methyl}-2- |
| | phenylcyclopropane-1-carboxamide |
| 2429. | 1-ethyl-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-1H-pyrrole-3- |
| | carboxamide |
| 2430. | 1-ethyl-N-{[3-(4-{[(3R,4S)-3-fluoro-1-methylpiperidin-4-yl]amino}-1-(2,2,2- |
| | trifluoroethyl)-1H-indol-2-yl)-1,2,4-oxadiazol-5-yl]methyl}-1H-pyrrole-3- |
| | carboxamide |
| 2431. | (+/−)-N-[(3R,4S)-3-fluoropiperidin-4-yl]-2-(5-{[(4-methanesulfonyl-2- |
| | methoxyphenyl)amino]methyl}-1,2,4-oxadiazol-3-yl)-1-(2,2,2-trifluoroethyl)-1H- |
| | indol-4-amine |
| 2432. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}thiophene-3-carboxamide |
| 2433. | N-{[3-(4-{[(3R,4S)-3-fluoro-1-methylpiperidin-4-yl]amino}-1-(2,2,2-trifluoroethyl)- |
| | 1H-indol-2-yl)-1,2,4-oxadiazol-5-yl]methyl}thiophene-3-carboxamide |
| 2434. | (+/−)-N-[(3R,4S)-3-fluoropiperidin-4-yl]-2-(5-{[methyl(1H-pyrazol-4- |
| | yl)amino]methyl}-1,2,4-oxadiazol-3-yl)-1-(2,2,2-trifluoroethyl)-1H-indol-4-amine |
| 2435. | (+/−)-N-[(3R,4S)-3-fluoro-1-methylpiperidin-4-yl]-2-(5-{[methyl(1H-pyrazol-4- |
| | yl)amino]methyl}-1,2,4-oxadiazol-3-yl)-1-(2,2,2-trifluoroethyl)-1H-indol-4-amine |
| 2436. | (1RS,2RS)-2-cyano-N-{[3-(4-{[(3S,4R)-3-fluoro-1-methylpiperidin-4-yl]amino}-1- |
| | (2,2,2-trifluoroethyl)-1H-indol-2-yl)-1,2,4-oxadiazol-5-yl]methyl}cyclopropane-1- |
| | carboxamide |
| 2437. | 1-tert-butyl-N-{[3-(4-{[(3 S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-1H-pyrrole-3- |
| | carboxamide |
| 2438. | (1RS,2SR)-2-cyano-N-{[3-(4-{[(3S,4R)-3-fluoro-1-methylpiperidin-4-yl]amino}-1- |
| | (2,2,2-trifluoroethyl)-1H-indol-2-yl)-1,2,4-oxadiazol-5-yl]methyl}cyclopropane-1- |
| | carboxamide |
| 2439. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}indolizine-2-carboxamide |
| 2440. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-phenyl-1H-imidazole-4-carboxamide |
| 2441. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-(2-methoxyethyl)-1H-pyrrole-3- |
| | carboxamide |
| 2442. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-(propan-2-yl)-1H-pyrrole-3- |
| | carboxamide |
| 2443. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}pyridine-4-carboxamide |
| 2444. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}pyridine-2-carboxamide |
| 2445. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}pyridine-3-carboxamide |
| 2446. | N-[(3S,4R)-3-fluoropiperidin-4-yl]-2-(5-{[(1H-pyrazol-4-yl)amino]methyl}-1,2,4- |
| | oxadiazol-3-yl)-1-(2,2,2-trifluoroethyl)-1H-indol-4-amine |
| 2447. | benzyl N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}carbamate |
| 2448. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-(2-fluoroethyl)-1H-pyrrole-3- |
| | carboxamide |
| 2449. | (1S,2R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-phenylcyclopropane-1- |
| | carboxamide |
| 2450. | (1R,2S)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-phenylcyclopropane-1- |
| | carboxamide |
| 2451. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-5-methylthiophene-3-carboxamide |
| 2452. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-4-methylthiophene-3-carboxamide |
| 2453. | (1R,2R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-(4- |
| | fluorophenyl)cyclopropane-1-carboxamide |
| 2454. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-2-methylthiophene-3-carboxamide |
| 2455. | (1s,3r)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-3-methylcyclobutane-1- |
| | carboxamide |
| 2456. | 5-chloro-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}thiophene-3-carboxamide |
| 2457. | N-[(3S,4R)-3-fluoropiperidin-4-yl]-2-(5-{[(1H-pyrazol-5-yl)amino]methyl}-1,2,4- |
| | oxadiazol-3-yl)-1-(2,2,2-trifluoroethyl)-1H-indol-4-amine |
| 2458. | 2-chloro-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}thiophene-3-carboxamide |
| 2459. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}pyrazolo[1,5-a]pyridine-2-carboxamide |
| 2460. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}imidazo[1,2-a]pyridine-2-carboxamide |
| 2461. | 1-cyclopropyl-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-1H-pyrrole-3- |
| | carboxamide |
| 2462. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1,5-dimethyl-1H-pyrrole-3-carboxamide |
| 2463. | 4-(dimethylamino)-N-{[3-(4-{[(3S,4R)-3-fluoro-1-methylpiperidin-4-yl]amino}-1- |
| | (2,2,2-trifluoroethyl)-1H-indol-2-yl)-1,2,4-oxadiazol-5-yl]methyl}benzamide |
| 2464. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}benzenesulfonamide |
| 2465. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}cyclobutanecarboxamide |
| 2466. | (1r,3s)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-3-methylcyclobutane-1- |
| | carboxamide |
| 2467. | (1R,2R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-(2- |
| | fluorophenyl)cyclopropane-1-carboxamide |
| 2468. | (1R,2R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-(1-methyl-1H-pyrazol- |
| | 4-yl)cyclopropane-1-carboxamide |
| 2469. | (1R,2R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-(pyridin-2- |
| | yl)cyclopropane-1-carboxamide |
| 2470. | N-[(3S,4R)-3-fluoro-1-methylpiperidin-4-yl]-2-(5-{[(1H-pyrazol-4-yl)amino]methyl}- |
| | 1,2,4-oxadiazol-3-yl)-1-(2,2,2-trifluoroethyl)-1H-indol-4-amine |
| 2471. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}acetamide |
| 2472. | 1-[2-(dimethylamino)ethyl]-N-{[3-(4-{[(3S,4R)-3-fluoro-1-methylpiperidin-4- |
| | yl]amino}-1-(2,2,2-trifluoroethyl)-1H-indol-2-yl)-1,2,4-oxadiazol-5-yl]methyl}-1H- |
| | pyrrole-3-carboxamide |
| 2473. | N-[(3S,4R)-3-fluoro-1-methylpiperidin-4-yl]-2-(5-{[(1H-pyrazol-5-yl)amino]methyl}- |
| | 1,2,4-oxadiazol-3-yl)-1-(2,2,2-trifluoroethyl)-1H-indol-4-amine |
| 2474. | 1-tert-butyl-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-1H-pyrazole-4- |
| | carboxamide |
| 2475. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-(propan-2-yl)-1H-imidazole-4- |
| | carboxamide |
| 2476. | (1S)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2,2- |
| | dimethylcyclopropane-1-carboxamide |
| 2477. | (1R,2R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-[(4-methylpiperazin-1- |
| | yl)methyl]cyclopropane-1-carboxamide |
| 2478. | (1R,2R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-(3- |
| | fluorophenyl)cyclopropane-1-carboxamide |
| 2479. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-methyl-1H-12,3-triazole-4- |
| | carboxamide |
| 2480. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-(1-hydroxypropan-2-yl)-1H-pyrrole-3- |
| | carboxamide |
| 2481. | 2-[3-({[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}carbamoyl)-1H-pyrrol-1- |
| | yl]acetic acid |
| 2482. | (1R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2,2- |
| | dimethylcyclopropane-1-carboxamide |
| 2483. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-(2-methylpropyl)-1H-pyrrole-3- |
| | carboxamide |
| 2484. | 1-(cyclopropylmethyl)-N-{[3-(4-{[(3S,4R)-3-fluoro-1-methylpiperidin-4-yl]amino}-1- |
| | (2,2,2-trifluoroethyl)-1H-indol-2-yl)-1,2,4-oxadiazol-5-yl]methyl}-1H-pyrrole-3- |
| | carboxamide |
| 2485. | 3-(dimethylamino)-N-{[3-(4-{[(3S,4R)-3-fluoro-1-methylpiperidin-4-yl]amino}-1- |
| | (2,2,2-trifluoroethyl)-1H-indol-2-yl)-1,2,4-oxadiazol-5-yl]methyl}benzamide |
| 2486. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-4-(pyrrolidin-1-yl)benzamide |
| 2487. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-(2-hydroxypropyl)-1H-pyrrole-3- |
| | carboxamide |
| 2488. | (1R,2R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-[(morpholin-4- |
| | yl)methyl]cyclopropane-1-carboxamide |
| 2489. | (1R,2R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-{[(propan-2- |
| | yl)amino]methyl}cyclopropane-1-carboxamide |
| 2490. | (1R,2R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2- |
| | [(propylamino)methyl]cyclopropane-1-carboxamide |
| 2491. | 3-[3-({[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}carbamoyl)-1H-pyrrol-1- |
| | yl]propanoic acid |
| 2492. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-(2-hydroxyethyl)-1H-pyrrole-3- |
| | carboxamide |
| 2493. | 1-(2,2-difluoroethyl)-N-{[3-(4-{[(3S,4R)-3-fluoro-1-methylpiperidin-4-yl]amino}-1- |
| | (2,2,2-trifluoroethyl)-1H-indol-2-yl)-1,2,4-oxadiazol-5-yl]methyl}-1H-pyrrole-3- |
| | carboxamide |
| 2494. | N-{[3-(4-{[(3S,4R)-3-fluoro-1-methylpiperidin-4-yl] amino}-1-(2,2,2-trifluoroethyl)- |
| | 1H-indol-2-yl)-1,2,4-oxadiazol-5-yl]methyl}-1-(2-methoxy-2-methylpropyl)-1H- |
| | pyrrole-3-carboxamide |
| 2495. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-(2-hydroxy-2-methylpropyl)-1H- |
| | pyrrole-3-carboxamide |
| 2496. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-(2-methoxypropyl)-1H-pyrrole-3- |
| | carboxamide |
| 2497. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-methyl-1H-indole-6-carboxamide |
| 2498. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-(propan-2-yl)-1H-pyrazole-4- |
| | carboxamide |
| 2499. | 4-(4,4-difluoropiperidin-1-yl)-N-{[3-(4-{[(3S,4R)-3-fluoro-1-methylpiperidin-4- |
| | yl]amino}-1-(2,2,2-trifluoroethyl)-1H-indol-2-yl)-1,2,4-oxadiazol-5- |
| | yl]methyl}benzamide |
| 2500. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-(propan-2-yl)-1H-imidazole-5- |
| | carboxamide |
| 2501. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1H-indole-5-carboxamide |
| 2502. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-methyl-1H-indole-5-carboxamide |
| 2503. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-4-(morpholin-4-yl)benzamide |
| 2504. | 2-[5-(aminomethyl)-1,2,4-oxadiazol-3-yl]-N-[(3S,4R)-3-fluoro-1-methylpiperidin-4- |
| | yl]-1-(2,2,2-trifluoroethyl)-1H-indol-4-amine |
| 2505. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-3-(morpholin-4-yl)benzamide |
| 2506. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-3-(pyrrolidin-1-yl)benzamide |
| 2507. | (1R,2R)-2-[(dimethylamino)methyl]-N-{[3-(4-{[(3S,4R)-3-fluoro-1-methylpiperidin- |
| | 4-yl]amino}-1-(2,2,2-trifluoroethyl)-1H-indol-2-yl)-1,2,4-oxadiazol-5- |
| | yl]methyl}cyclopropane-1-carboxamide |
| 2508. | (1R,2R)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-[(pyrrolidin-1- |
| | yl)methyl]cyclopropane-1-carboxamide |
| 2509. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-3-methyl-1H-indole-5-carboxamide |
| 2510. | N-{[3-(4-{[(3S,4R)-3-fluoro-1-methylpiperidin-4-yl] amino}-1-(2,2,2-trifluoroethyl)- |
| | 1H-indol-2-yl)-1,2,4-oxadiazol-5-yl]methyl}-1H-indole-6-carboxamide |
| 2511. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-1-(2-methoxyethyl)-1H-imidazole-5- |
| | carboxamide |
| 2512. | (1R,2R)-2-ethyl-N-{[3-(4-{[(3S,4R)-3-fluoro-1-methylpiperidin-4-yl]amino}-1- |
| | (2,2,2-trifluoroethyl)-1H-indol-2-yl)-1,2,4-oxadiazol-5-yl]methyl}cyclopropane-1- |
| | carboxamide |
| 2513. | 1-tert-butyl-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-1H-imidazole-5- |
| | carboxamide |
| 2514. | (1S,2S)-N-{[3-(4-{[(3S,4R)-3-fluoro-1-methylpiperidin-4-yl]amino}-1-(2,2,2- |
| | trifluoroethyl)-1H-indol-2-yl)-1,2,4-oxadiazol-5-yl]methyl}-2-{[(propan-2- |
| | yl)amino]methyl}cyclopropane-1-carboxamide |
| 2515. | N-{[3-(4-{[(3S,4R)-1-ethyl-3-fluoropiperidin-4-yl]amino}-1-(2,2,2-trifluoroethyl)- |
| | 1H-indol-2-yl)-1,2,4-oxadiazol-5-yl]methyl}-1-(2-methoxyethyl)-1H-pyrrole-3- |
| | carboxamide |
| 2516. | N-{[3-(4-{[(3R,4S)-1-ethyl-3-fluoropiperidin-4-yl]amino}-1-(2,2,2-trifluoroethyl)- |
| | 1H-indol-2-yl)-1,2,4-oxadiazol-5-yl]methyl}-1-(2-methoxyethyl)-1H-pyrrole-3- |
| | carboxamide |
| 2517. | N-{[3-(4-{[(3S,4R)-3-fluoro-1-methylpiperidin-4-yl]amino}-1-(2,2,2-trifluoroethyl)- |
| | 1H-indol-2-yl)-1,2,4-oxadiazol-5-yl]methyl}-3-methyl-1H-indole-6-carboxamide |
| 2518. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,2,4-oxadiazol-3-yl]methyl}cyclopropanecarboxamide |
| 2519. | (+/−)-N-{[5-(4-{[(3R,4S)-3-fluoro-1-methylpiperidin-4-yl] amino}-1-(2,2,2- |
| | trifluoroethyl)-1H-indol-2-yl)-1,2,4-oxadiazol-3-yl]methyl}benzamide |
| 2520. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,2,4-oxadiazol-3-yl]methyl}thiophene-2-carboxamide |
| 2521. | (+/−)-N-{[5-(4-{[(3R,4S)-3-fluoro-1-methylpiperidin-4-yl]amino}-1-(2,2,2- |
| | trifluoroethyl)-1H-indol-2-yl)-1,2,4-oxadiazol-3-yl]methyl}thiophene-3-carboxamide |
| 2522. | (+/−)-N-{[5-(4-{[(3R,4S)-3-fluoro-1-methylpiperidin-4-yl] amino}-1-(2,2,2- |
| | trifluoroethyl)-1H-indol-2-yl)-1,2,4-oxadiazol-3-yl]methyl}-1,3-thiazole-5- |
| | carboxamide |
| 2523. | N-((3-(4-(((3S,4R)-3-fluoro-1-methylpiperidin-4-yl)amino)-1-(2,2,2-trifluoroethyl)- |
| | 1H-indol-2-yl)-1,2,4-oxadiazol-5-yl)methyl)-1-(1-fluoropropan-2-yl)-1H-pyrrole-3- |
| | carboxamide |
| 2524. | N-((3-(4-(((3S,4R)-1-ethyl-3-fluoropiperidin-4-yl)amino)-1-(2,2,2-trifluoroethyl)-1H- |
| | indol-2-yl)-1,2,4-oxadiazol-5-yl)methyl)-1-(2-fluoroethyl)-1H-pyrrole-3-carboxamide |
| 2525. | N-((3-(4-(((3S,4R)-3-fluoro-1-methylpiperidin-4-yl)amino)-1-(2,2,2-trifluoroethyl)- |
| | 1H-indol-2-yl)-1,2,4-oxadiazol-5-yl)methyl)-1-(1-methoxypropan-2-yl)-1H-pyrrole-3- |
| | carboxamide |

TABLE 4
Compounds substituted with at least one deuterium atom of the disclosure.

| Mol # | IUPAC name |
|---|---|
| 2526. | 4-[3-[4-[[(3R,4S)-1-tert-butyl-3-fluoro-4-piperidyl]amino]-1-(2,2,2- |
| | trifluoroethyl)indol-2-yl]prop-2-ynylamino]-N-methyl-3-(trideuteriomethoxy) |
| | benzamide |
| 2527. | 4-[3-[4-[(3S,4R)-1-tert-butyl-3-fluoro-4-piperidyl]amino]-1-(2,2,2- |
| | trifluoroethyl)indol-2-yl]prop-2-ynylamino]-N-methyl-3-(trideuteriomethoxy) |
| | benzamide |
| 2528. | 4-[3-[4-[[(3S,4R)-3-fluoro-1-methyl-4-piperidyl]amino]-1-(2,2,2- |
| | trifluoroethyl)indol-2-yl]prop-2-ynylamino]-N-methyl-3- |
| | (trideuteriomethoxy)benzamide |
| 2529. | 4-[3-[4-[[(3S,4R)-3-fluoro-1-methyl-4-piperidyl]amino]-1-(2,2,2- |
| | trifluoroethyl)indol-2-yl]prop-2-ynylamino]-3-(trideuteriomethoxy)-N- |
| | (trideuteriomethyl)benzamide |
| 2530. | 4-[3-[4-[[(3S,4R)-3-fluoro-1-methyl-4-piperidyl]amino]-1-(2,2,2- |
| | trifluoroethyl)indol-2-yl]prop-2-ynylamino]-3-methoxy-N- |
| | (trideuteriomethyl)benzamide |
| 2531. | rac-N-[(3S,4R)-3-fluoro-1-(trideuteriomethyl)-4-piperidyl]-2-[3-[4-methylsulfonyl- |
| | 2-(trideuteriomethoxy)anilino]prop-1-ynyl]-1-(2,2,2-trifluoroethyl)indol-4- |
| | ylamine |
| 2532. | [(3S,4R)-3-Fluoro-1-methyl-4-piperidyl](2-{4-[2-methoxy-4- |
| | (methylsulfonyl)phenyl]-1-butynyl}-1-(2,2,2-trifluoroethyl)-1H-indol-4-yl)amine |
| 2533. | rac-N-((3R,4S)-3-fluoro-1-methylpiperidin-4-yl)-2-(3-((2-trideuteriomethoxy-4- |
| | (methylsulfonyl)phenyl)amino)prop-1-yn-1-yl)-1-(2,2,2-trifluoroethyl)-1H-indol- |
| | 4-amine |
| 2534. | (1-trideuteriomethyl-4-piperidyl)(2-{4-[2-methoxy-4-(methylsulfonyl)phenyl]-1- |
| | butynyl}-1-(2,2,2-trifluoroethyl)-1H-indol-4-yl)amine |
| 2535. | 3-methoxy-1-[4-(2-{4-[4-(methylsulfonyl)-2-(trideuteriomethoxy)phenyl]-1- |
| | butynyl}-1-(2,2,2-trifluoroethyl)-1H-indol-4-ylamino)-1-piperidyl]-2-propanol |
| 2536. | 3-(trideuteriomethoxy)-1-[4-(2-{4-[2-methoxy-4-(methylsulfonyl)phenyl]-1- |
| | butynyl}-1-(2,2,2-trifluoroethyl)-1H-indol-4-ylamino)-1-piperidyl]-2-propanol |
| 2537. | 4-[3-[4-[[(3R,4S)-3-fluoro-1-methyl-4-piperidyl]amino]-1-(2,2,2- |
| | trifluoroethyl)indol-2-yl]prop-2-ynylamino]-N-methyl-3- |
| | (trideuteriomethoxy)benzamide |
| 2538. | 5-((3-(4-(((3S,4R)-3-fluoro-1-methylpiperidin-4-yl)amino)-1-(2,2,2- |
| | trifluoroethyl)-1H-indol-2-yl)prop-2-yn-1-yl)amino)-6-(trideuteriomethoxy)-2,3- |
| | dihydrobenzo[d]isothiazole 1,1-dioxide |
| 2539. | 4-((3-(4-(((3S,4R)-3-fluoro-1-methylpiperidin-4-yl)amino)-1-(2,2,2- |
| | trifluoroethyl)-1H-indol-2-yl)prop-2-yn-1-yl)amino)-3- |
| | (trideuteriomethoxy)benzenesulfonamide |
| 2540. | N-((3S,4R)-3-fluoro-1-methylpiperidin-4-yl)-2-(3-((2-(trideuteriomethoxy)-4- |
| | ((trideuteriomethyl)sulfonyl)phenyl)amino)prop-1-yn-1-yl)-1-(2,2,2- |
| | trifluoroethyl)-1H-indol-4-amine |
| 2541. | 4-((3-(4-(((3S,4R)-3-fluoro-1-(trideuteriomethyl)piperidin-4-yl)amino)-1-(2,2,2- |
| | trifluoroethyl)-1H-indol-2-yl)prop-2-yn-1-yl)amino)-3- |
| | (trideuteriomethoxy)benzenesulfonamide |
| 2542. | 4-((3-(4-(((3S,4R)-3-fluoro-1-(trideuteriomethyl)piperidin-4-yl)amino)-1-(2,2,2- |
| | trifluoroethyl)-1H-indol-2-yl)prop-2-yn-1-yl)amino)-3- |
| | methoxybenzenesulfonamide |
| 2543. | 4-((3-(4-(((3S,4R)-3-fluoro-1-(trideuteriomethyl)piperidin-4-yl)amino)-1-(2,2,2- |
| | trifluoroethyl)-1H-indol-2-yl)prop-2-yn-1-yl)amino)-3- |
| | methoxybenzenesulfonamide |
| 2544. | 5-((3-(4-(((3S,4R)-3-fluoro-1-(trideuteriomethyl)piperidin-4-yl)amino)-1-(2,2,2- |
| | trifluoroethyl)-1H-indol-2-yl)prop-2-yn-1-yl)amino)-6-methoxy-2,3- |
| | dihydrobenzo[d]isothiazole 1,1-dioxide |
| 2545. | (4-((3-(4-(((3S,4R)-3-fluoro-1-(trideuteriomethyl)piperidin-4-yl)amino)-1-(2,2,2- |
| | trifluoroethyl)-1H-indol-2-yl)prop-2-yn-1-yl)amino)-3- |
| | methoxyphenyl)dimethylphosphine oxide |
| 2546. | (4-((3-(4-(((3S,4R)-3-fluoro-1-methylpiperidin-4-yl)amino)-1-(2,2,2- |
| | trifluoroethyl)-1H-indol-2-yl)prop-2-yn-1-yl)amino)-3- |
| | (trideuteriomethoxy)phenyl)dimethylphosphine oxide |

### Example 2: Treatment with Compound 1 Reactivates Y220C p53 and Results in Tumor T-Cell Increases and Macrophage, g-MDSC Decreases

Compound 1 is an indole compound substituted with a trifluoroethyl group at the 1-position; propynyl amino-methoxy-methylamido phenyl group at the 2-position; and a heterocycle-substituted amino group at the 4-position. Six days post inoculation with MVT373 cells, mice were administered Compound 1 at 75 mg/kg, 150 mg/kg, or 300 mg/kg for 2Q7Dx1, 2Q7Dx2, or 2Q7Dx3. Tumors were harvested 72 h post dose for analysis of tumor infiltrating lymphocytes by flow cytometry. Reactivation of p53 led to increases in CD4+, CD8+, T-Regulatory T-cells and NKT cells and decreases in g-MDSC and M2 macrophages that were dose and time dependent. The data showed that administering Compound 1 increased immune responses in a dose and time dependent manner, which were demonstrated by observed changes in immune cell populations.

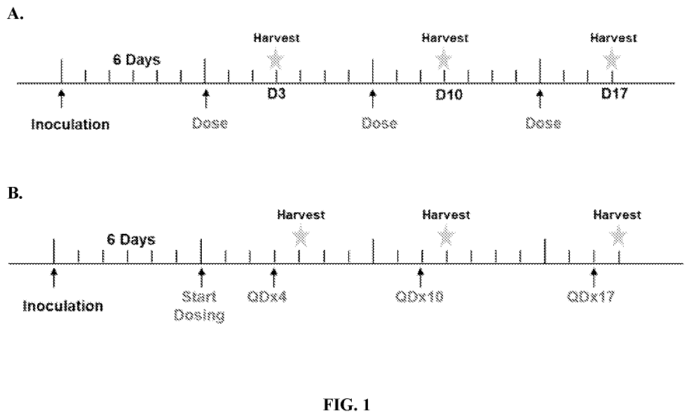

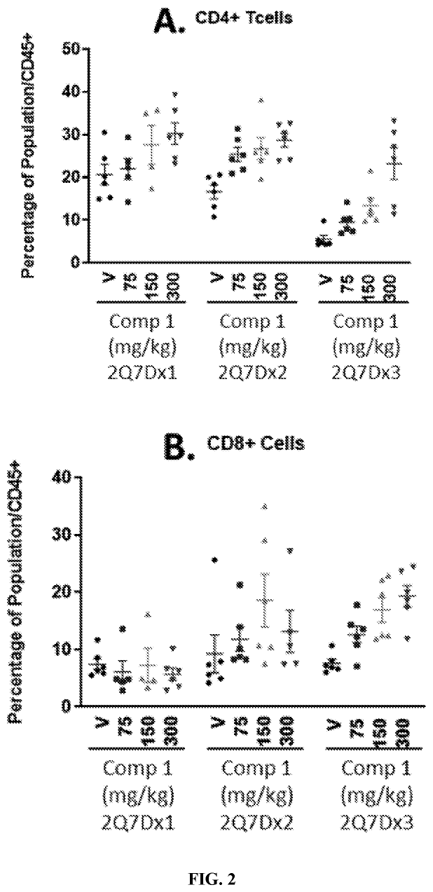

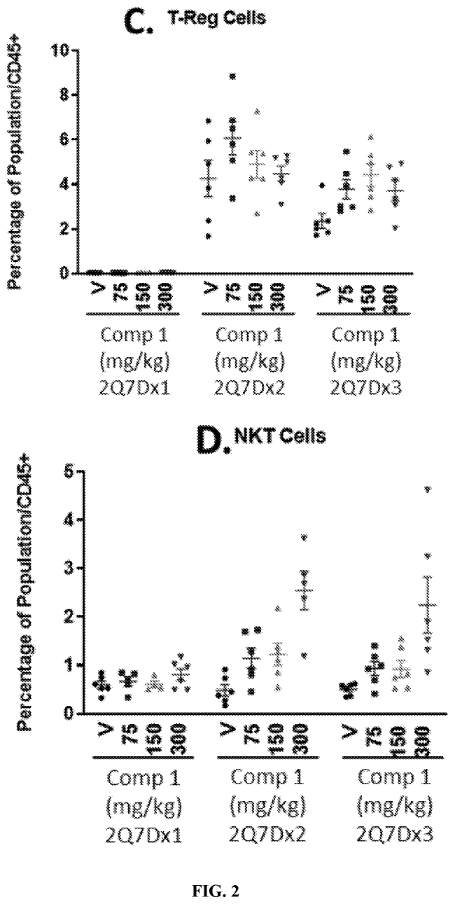

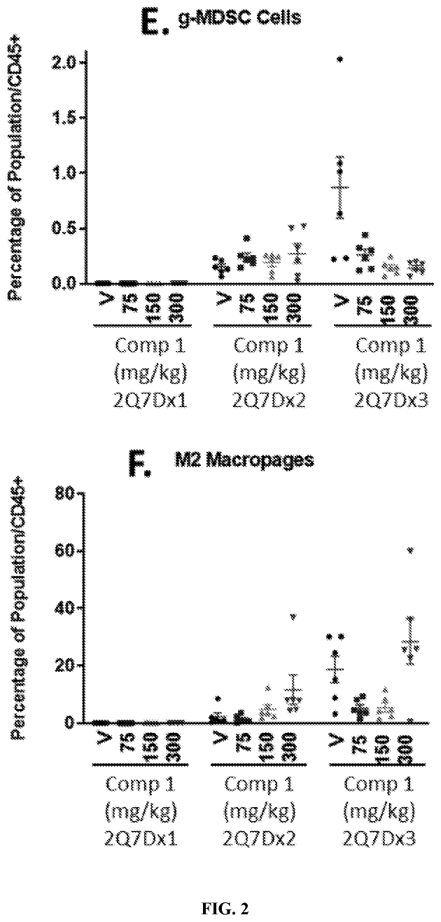

FIG. 1 PANEL A shows the dosing and tumor harvesting timeline of EXAMPLE 2. FIG. 2 PANEL A-PANEL F show changes in percentage of population of CD4+ T-cells (FIG. 2 PANEL A), CD8+ cells (FIG. 2 PANEL B), T-reg cells (FIG. 2 PANEL C), NKT cells (FIG. 2 PANEL D), g-MDSC cells (FIG. 2 PANEL E), and M2 macrophages (FIG. 2 PANEL F) compared to CD45+ in mice treated with vehicle, 75 mg/kg, 150 mg/kg, and 300 mg/kg of Compound 1 for 2Q7Dx1, 2Q7Dx2, and 2Q7Dx3.

### Example 3: Treatment with Compound 2 Reactivates Y220C p53 and Results in Tumor T-Cell Increases and Macrophage, g-MDSC Decreases

Compound 2 is an indole compound substituted with a trifluoroethyl group at the 1-position; propynyl amino-methoxy-methylsulfonyl phenyl group at the 2-position; and a heterocycle-substituted amino group at the 4-position. Six days post inoculation with MT373 cells, mice were administered Compound 2 at 25 mg/kg, 50 mg/kg, or 100 mg/kg QD for 4, 10 or 17 doses. Tumors were harvested 24 h post dose for analysis of tumor infiltrating lymphocytes by flow cytometry. Reactivation of p53 led to increases in CD4+, CD8+, T-Regulatory T-cells and NKT cells and decreases in g-MDSC and M2 macrophages that were dose responsive and time dependent. The data showed that administering Compound 2 increased immune responses in a dose and time dependent manner, which were demonstrated by observed changes in immune cell populations.

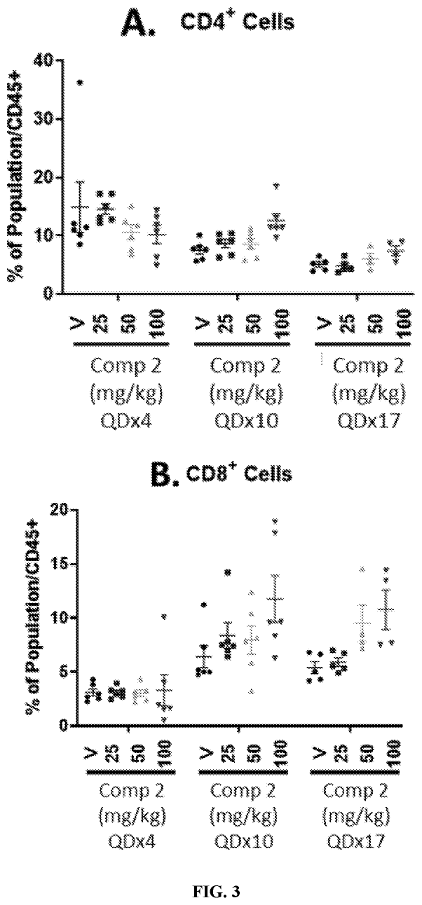

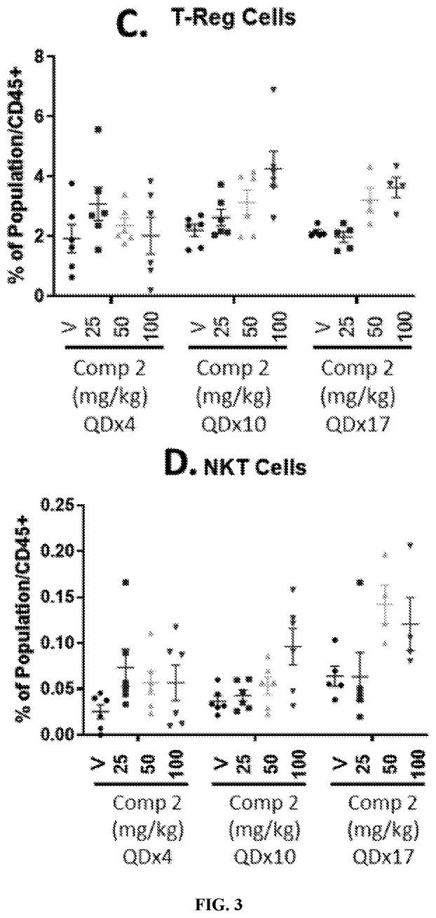

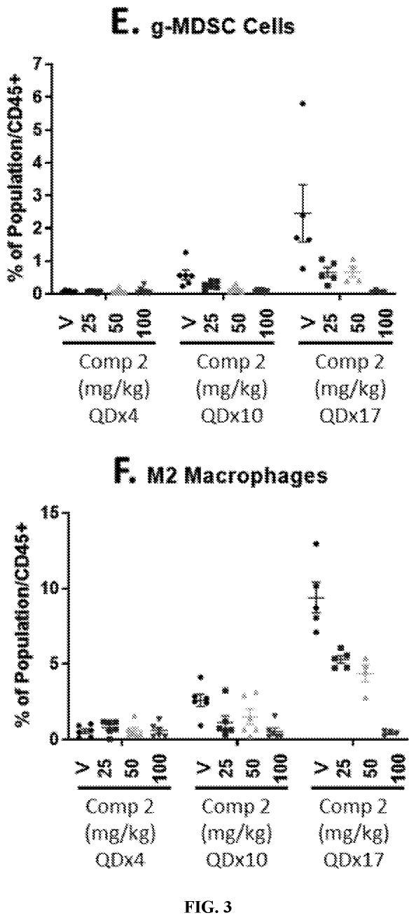

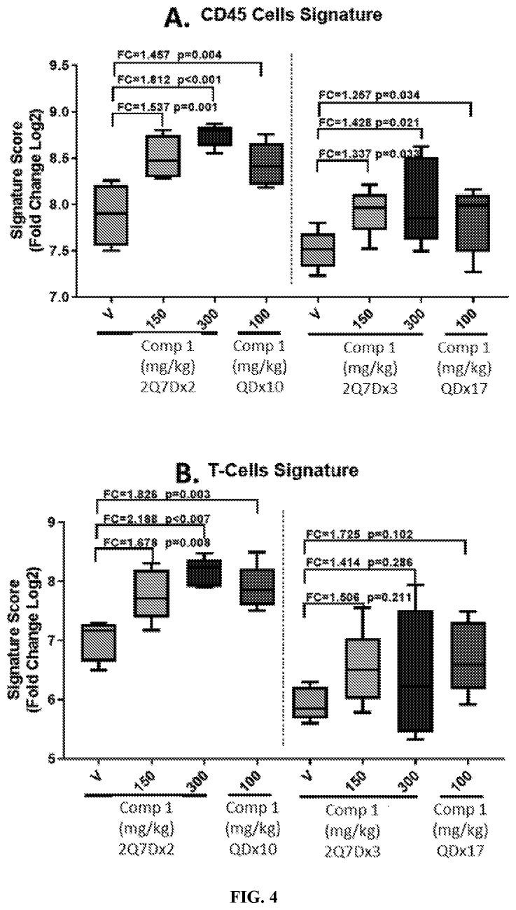

FIG. 1 PANEL B shows the dosing and tumor harvesting timeline of EXAMPLE 3. FIG. 3 PANEL A-PANEL F show changes in percentage of population of CD4+ T-cells (FIG. 3 PANEL A), CD8+ cells (FIG. 3 PANEL B), T-reg cells (FIG. 3 PANEL C), NKT cells (FIG. 3 PANEL D), g-MDSC cells (FIG. 3 PANEL E), and M2 macrophages (FIG. 3 PANEL F) compared to CD45+ in mice treated with vehicle, 25 mg/kg, 50 mg/kg, and 100 mg/kg of Compound 2 for QDx4, QDx10, and QDx17.

### Example 4: Increase in T-Cell Signatures Correlate with Flow Cytometry Data; Increases in TIS Signature and Check-Point Biomarkers are Consistent with Enhanced Combination Efficacy

Six days post inoculation with MT373 cells, mice were administered Compound 1 at 150 mg/kg or 300 mg/kg 2Q7Dx2 or 2Q7Dx3 and 100 mg/kg QDx10 or QDx17. Tumor samples were analyzed by NanoString for gene expression analysis. Fold change (FC) and p-values were relative to vehicle (V) control. Reactivation of p53 led to increases in CD45⁺, T-cell and exhausted CD8 signatures that were consistent with the increase in similar populations as observed with flow cytometry and correlated with an immune response with p53 reactivation. Increases in Tumor Inflammation Signature (TIS), PDL1 Signature, and PD-1 signature with p53 reactivation were consistent with the synergistic effect seen with anti-PD1 response in the combination efficacy studies.

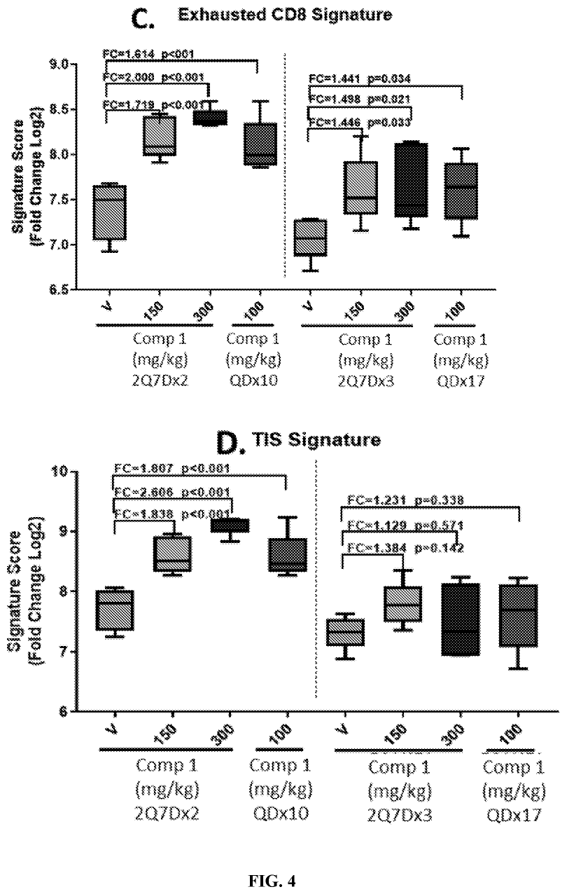

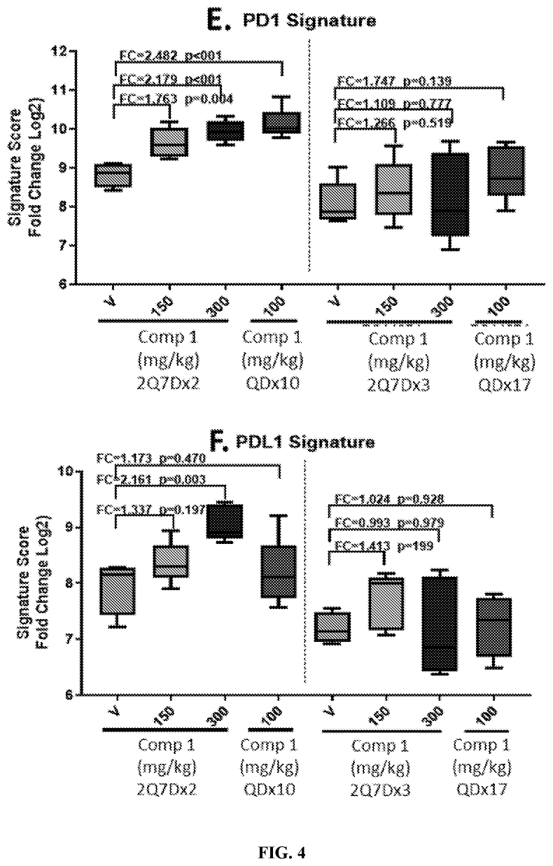

FIG. 4 PANEL A-PANEL F show changes in NanoString signature scores for CD45 cell signature analysis (FIG. 4 PANEL A), T-cell signature analysis (FIG. 4 PANEL B), exhausted CD8 signature analysis (FIG. 4 PANEL C), tumor inflammation signature analysis (FIG. 4 PANEL D), PD1 signature analysis (FIG. 4 PANEL E), and PDL1 signature analysis (FIG. 4 PANEL F) in mice treated with vehicle, 150 mg/kg and 300 mg/kg of Compound 1 at 2Q7Dx2 and 2Q7Dx3; or 100 mg/kg of Compound 1 at QDx10 and QDx17.

1-127. (canceled)

128. A method comprising:
a) determining that a subject is in need of an increase in a gene signature, wherein the gene signature comprises an immune response gene signature; and
b) based on the determining, administering a therapeutically-effective amount of a compound to the subject in need thereof, wherein the compound is 4-((3-(4-(((3S,4R)-3-fluoro-1-methylpiperidin-4-yl)amino)-1-(2,2,2-trifluoroethyl)-1H-indol-2-yl)prop-2-yn-1-yl)amino)-3-methoxy-N-methylbenzamide or a pharmaceutically-acceptable salt thereof.

129. The method of claim 128, wherein the subject has a tumor.

130. The method of claim 129, wherein the tumor comprises a Y220C mutation.

131. The method of claim 129, wherein the tumor is ovarian cancer.

132. The method of claim 129, wherein the tumor is breast cancer.

133. The method of claim 129, wherein the tumor is lung cancer.

134. The method of claim 129, wherein the tumor is endometrial cancer.

135. The method of claim 128, wherein the administering is oral.

136-137. (canceled)

138. The method of claim 128, wherein the therapeutically-effective amount is from about 1000 mg to about 3,000 mg.

139-140. (canceled)

141. The method of claim 128, wherein the administering is for at least about 1 week.

142-144. (canceled)

145. The method of claim 128, wherein the immune response gene signature is a tumor inflammation signature.

146. The method of claim 128, wherein the immune response gene signature is a CD45 cell signature.

147. The method of claim 128, wherein the immune response gene signature is a T-cell signature.

148. The method of claim 128, wherein the immune response gene signature is an exhausted CD8 signature.

149. The method of claim 128, wherein the gene signature comprises a check-point biomarker signature.

150. The method of claim 149, wherein the check-point biomarker signature is a PDL1 signature.

151. The method of claim 149, wherein the check-point biomarker signature is a PD-1 signature.

152-176. (canceled)

177. The method of claim 128, further comprising administering a therapeutically-effective amount of an anti-cancer agent.

178. The method of claim 177, wherein the anti-cancer agent is an immune checkpoint inhibitor.

179-182. (canceled)

183. The method of claim 177, wherein the therapeutically-effective amount of the anti-cancer agent is from about 1 pig/kg to about 10 mg/kg.

184. (canceled)


SEQUENCE LISTING (1 sequence)

SEQ ID NO: 1 - protein, 219 aa, synthetic construct
SSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPCEPPEVGSDCTTIHYNYMCNSSCMGGMNRRPILTIITLEDSSGNLLGRNSFEVHVCACPGRDRRTEEENLRKKGEPHHELPPGSTKRALSNNT